(19) United States
(12) Patent Application Publication
(10) Pub. No.: US 2024/0334814 A1
(43) Pub. Date: Oct. 3, 2024

(54) MIXED POWDER, MIXTURE, AND METHOD FOR FABRICATING ORGANIC ELECTROLUMINESCENCE DEVICE

(71) Applicant: IDEMITSU KOSAN Co.,LTD., Tokyo (JP)

(72) Inventors: Tetsuya MASUDA, Tokyo (JP); Hiroyuki IWABUCHI, Tokyo (JP); Masato NAKAMURA, Tokyo (JP); Yoshinao SHIRASAKI, Tokyo (JP); Satomi TASAKI, Tokyo (JP); Hiroaki TOYOSHIMA, Tokyo (JP)

(73) Assignee: IDEMITSU KOSAN Co.,LTD., Tokyo (JP)

(21) Appl. No.: 18/574,016

(22) PCT Filed: Jun. 17, 2022
(86) PCT No.: PCT/JP2022/024294

§ 371 Date: Dec. 23, 2023

Related U.S. Application Data
(63) Continuation-in-part of application No. PCT/JP22/24294, filed on Nov. 5, 2021.

(30) Foreign Application Priority Data
Jun. 25, 2021 (JP) 2021-105558
Jun. 25, 2021 (JP) 2021-105763
Apr. 1, 2022 (JP) 2022-061942

(51) Int. Cl. H10K 85/60; C07B 59/00; H10K 71/16; H10K 85/20; H10K 85/40

(52) U.S. Cl. CPC H10K 85/622; C07B 59/004; H10K 71/164; H10K 85/20; H10K 85/40; H10K 85/623; H10K 85/633; H10K 85/653; H10K 85/654; H10K 85/6572; H10K 85/6574; H10K 85/6576; H10K 85/658; C07B 2200/05

(57) ABSTRACT

A mixed powder comprising a first compound and a second compound, wherein the first compound has the fused ring structure having the most rings and having three or more rings, in fused ring structures included therein, and the second compound has the fused ring structure having the most rings and having 5 or more rings, in fused ring structures included therein, and the fused ring structure having the most rings thereof has more rings than that of the first compound.

### TECHNICAL FIELD

The present invention relates to a mixed powder, a mixture, and a method for fabricating an organic electroluminescence device.

### BACKGROUND ART

As a method for forming an organic layer constituting an organic electroluminescence device (hereinafter, also referred to as “an organic EL device”), a vacuum deposition method is generally used, and, a co-deposition method has been conventionally used to form a mixed layer composed of a plurality of components, in which each component is vaporized and simultaneously deposited each in different deposition sources (crucibles).

Since it is possible in the co-deposition method to independently control the temperature for each deposition source, it is easy to adjust the mixing proportion in the deposited film by controlling the amount of vaporization of each material, and further, even when deposition is continuously conducted on a plurality of substrates, it is possible to form the film with a fixed mixing proportion. On the other hand, since the fabricating process thereof is complicated due to the use of multiple deposition sources, there is a problem that fabricating burden and costs are increased, and the like.

As a technique for solving the problem, a deposition technique in which a so-called premix material that a plurality of materials (organic compounds) is mixed in advance is vaporized in a single deposition source to form a film is attracting attention. As an example of the premix technique, Patent Document 1 discloses the following inventions.

1. A mixture (hereinafter, also referred to as the “mixture A”) comprising: a first compound; and a second compound, wherein the first compound comprises at least one of a first cyclic structure represented by the formula (11) or a second cyclic structure represented by the formula (12), the second compound is a compound represented by the formula (21) or a compound represented by the formula (22), and a total mass M_{T} of the first compound and the second compound and a mass M₂ of the second compound in the mixture satisfy the following Numerical formula 1:

$$0.1 ≦ ( {M}_{2} / {M}_{T} ) \times 100 \qquad ( Numerical formula 1 )$$

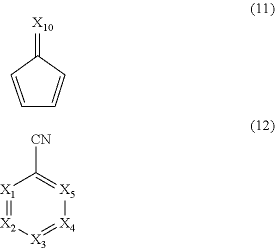

wherein: the first cyclic structure represented by the formula (11) is fused to at least one cyclic structure of a substituted or unsubstituted aromatic hydrocarbon ring having 6 to 50 ring carbon atoms or a substituted or unsubstituted heterocycle having 5 to 50 ring atoms in a molecule of the first compound, a structure represented by ═X₁₀ is represented by the formula (11a), (11b), (11c), (11d), (11e), (11f), (11g), (11h), (11i), (11j), (11k) or (11m):

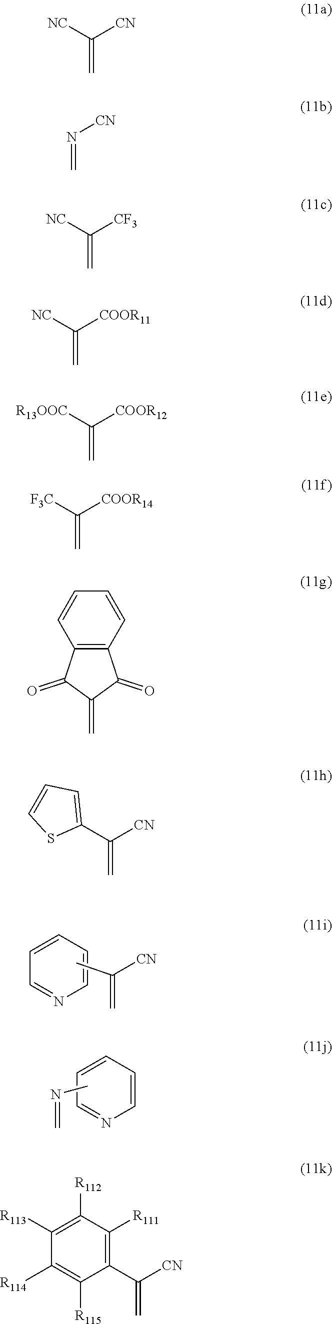

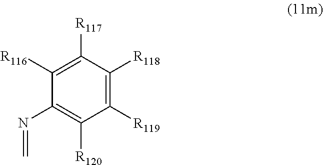

wherein in the formula (11a), (11b), (11c), (11d), (11e), (11f), (11g), (11h), (11i), (11j), (11k) or (11m): R₁₁ to R₁₄ and R₁₁₁ to R₁₂₀ are each independently a hydrogen atom, a halogen atom, hydroxy group, a cyano group, a substituted or unsubstituted alkyl group having 1 to 50 carbon atoms, a substituted or unsubstituted alkyl halide group having 1 to 50 carbon atoms, a substituted or unsubstituted cycloalkyl group having 3 to 50 ring carbon atoms, a group represented by —Si(R₉₀₁) (R₉₀₂) (R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms,

in the formula (12): X₁ to X₅ are each independently a nitrogen atom, a carbon atom to be bonded to R₁₅, or a carbon atom to be bonded to another atom in the molecule of the first compound; at least one of X₁ to X₅ is a carbon atom to be bonded to another atom in the molecule of the first compound; R₁₅ is selected from the group consisting of a hydrogen atom, a halogen atom, a cyano group, a substituted or unsubstituted alkyl group having 1 to 50 carbon atoms, a substituted or unsubstituted alkyl halide group having 1 to 50 carbon atoms, a substituted or unsubstituted cycloalkyl group having 3 to 50 ring carbon atoms, a substituted or unsubstituted aryl group having 6 to 50 ring carbon atoms, a substituted or unsubstituted heterocyclic group having 5 to 50 ring atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted alkenyl group having 2 to 50 carbon atoms, a substituted or unsubstituted aralkyl group having 7 to 50 carbon atoms, a carboxy group, a substituted or unsubstituted ester group, a substituted or unsubstituted carbamoyl group, a nitro group, and a substituted or unsubstituted siloxanyl group; and when a plurality of R₁₅ are present, the plurality of R₁₅ are mutually the same or different:

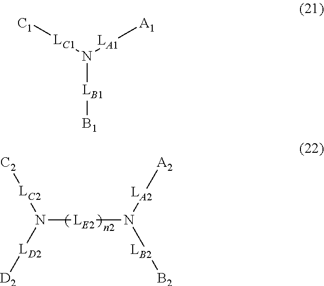

wherein in the formulas (21) and (22): L_{A1}, L_{B1}, L_{C1}, L_{A2}, L_{B2}, L_{C2} and L_{D2} are each independently a single bond, a substituted or unsubstituted arylene group having 6 to 50 ring carbon atoms, or a substituted or unsubstituted divalent heterocyclic group having 5 to 50 ring atoms; n2 is 1, 2, 3 or 4; when n2 is 1, L_{E2} is a substituted or unsubstituted arylene group having 6 to 50 ring carbon atoms, or a substituted or unsubstituted divalent heterocyclic group having 5 to 50 ring atoms; when n2 is 2, 3 or 4, a plurality of L_{E2} are mutually the same or different; when n2 is 2, 3 or 4, the plurality of L_{E2} are mutually bonded to form a substituted or unsubstituted monocyclic ring, mutually bonded to form a substituted or unsubstituted fused ring, or not mutually bonded; L_{E2} forming neither the monocyclic ring nor the fused ring is a substituted or unsubstituted arylene group having 6 to 50 ring carbon atoms, or a substituted or unsubstituted divalent heterocyclic group having 5 to 50 ring atoms; A₁, B₁, C₁, A₂, B₂, C₂ and D₂ are each independently a substituted or unsubstituted aryl group having 6 to 50 ring carbon atoms, a substituted or unsubstituted heterocyclic group having 5 to 50 ring atoms, or —Si(R′₉₀₁) (R′₉₀₂) (R′₉₀₃); R′₉₀₁, R′₉₀₂ and R′₉₀₃ are each independently a substituted or unsubstituted aryl group having 6 to 50 ring carbon atoms; when a plurality of R′₉₀₁ are present, the plurality of R′₉₀₁ are mutually the same or different; when a plurality of R′₉₀₂ are present, the plurality of R′₉₀₂ are mutually the same or different; when a plurality of R′₉₀₃ are present, the plurality of R′₉₀₃ are mutually the same or different;

in the first compound and the second compound, R₉₀₁ to R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms; when a plurality of R₉₀₁ are present, the plurality of R₉₀₁ are mutually the same or different; when a plurality of R₉₀₂ are present, the plurality of R₉₀₂ are mutually the same or different; when a plurality of R₉₀₃ are present, the plurality of R₉₀₃ are mutually the same or different; when a plurality of R₉₀₄ are present, the plurality of R₉₀₄ are mutually the same or different; when a plurality of R₉₀₅ are present, the plurality of R₉₀₅ are mutually the same or different; when a plurality of R₉₀₆ are present, the plurality of R₉₀₆ are mutually the same or different; and when a plurality of R₉₀₇ are present, the plurality of R₉₀₇ are mutually the same or different.

2. An organic electroluminescence device comprising: an anode; a cathode; an organic layer comprising a first compound and a second compound,

wherein the first compound comprises at least one of the first cyclic structure represented by the formula (11) or the second cyclic structure represented by the formula (12), the second compound is the compound represented by the formula (21) or the compound represented by the formula (22), a total mass W_{T} of the first compound and the second compound and a mass W₂ of the second compound in the organic layer satisfy the following Numerical formula 2:

$$0.1 ≦ ( {W}_{2} / {W}_{T} ) \times 100. \qquad ( Numerical formula 2 )$$

### RELATED ART DOCUMENTS

### Patent Documents

[Patent Document 1] WO 2021/015266 A1

### SUMMARY OF THE INVENTION

The premix technique can overcome the above-mentioned disadvantages of the co-deposition method, however, there are problems, as compared to the co-deposition method, that it is difficult to form a deposited film with a desired mixing proportion, and further that when a plurality of substrates is continuously subjected to deposition, the mixing proportion thereof is variable depending on the substrate to be difficult to obtain one with consistent quality. Since the deposition process is continuously conducted for several weeks to several months in the actual fabrication of organic EL devices, it is essential that mixed films can be formed with stable proportions for a long time. That is, the above-mentioned problems are important problems to be solved in the premix technique.

It is an object of the present invention to provide a mixed powder capable of deposition in which the variability of the component proportion is suppressed in the mixed film during the film formation process in the deposition process by using mixed materials including a plurality of compounds.

As a result of studies focusing on fused ring structures of compounds, the present inventors have found that the compounds having the specific fused ring structures are used in combination to be capable of solving the above-mentioned problem, and has completed the present invention.

According to the present invention, the following mixed powder and the like are provided.

1. A mixed powder comprising a first compound and a second compound,
wherein the first compound has the fused ring structure having the most rings and having three or more rings, in fused ring structures included therein, and
the second compound has the fused ring structure having the most rings and having 5 or more rings, in fused ring structures included therein, and the fused ring structure having the most rings thereof has more rings than that of the first compound.
   2. A method for fabricating an organic electroluminescence device, wherein the organic electroluminescence device comprises a cathode, an anode, and one or two or more organic layers arranged between the cathode and the anode, and comprising an emitting layer, and
the method comprises: depositing a mixed powder by heating and vaporizing the mixed powder which comprises a first compound and a second compound, which is solid at ordinary temperatures and pressures, and in which the first compound has the fused ring structure having the most rings and having three or more rings, in fused ring structures included therein, and the second compound has the fused ring structure having the most rings and having 5 or more rings, in fused ring structures included therein, and the fused ring structure having the most rings thereof has more rings than that of the first compound in a deposition source to form at least one layer of the one or two or more organic layers.

According to the present invention, there can be provided a mixed powder capable of deposition in which the variability of the component proportion is suppressed in the mixed film during the film formation process in the deposition process by using mixed materials including a plurality of compounds.

### BRIEF DESCRIPTION OF THE DRAWINGS

FIG. 1 is a diagram showing a deposition characteristic curve of each component in an example of a mixed powder according to an aspect of the present invention.

FIG. 2 is a diagram showing a schematic configuration of an organic EL device obtained by a method for fabricating an organic EL device according to an aspect of the present invention.

FIG. 3 is a deposition characteristic curve of temperature and P/M^{1/2} each for BH-1 and BD-1 used in Examples 1-1 and 1-2.

FIG. 4 is a deposition characteristic curve of temperature and P/M^{1/2} each for BH-2 and BD-2 used in Examples 2-1 and 2-2.

FIG. 5 is a deposition characteristic curve of temperature and P/M^{1/2} each for BH-3 and BD-3 used in Example 3.

FIG. 6 is a deposition characteristic curve of temperature and P/M^{1/2} each for HI-1 and HT-1 used in Comparative Example 1.

### MODE FOR CARRYING OUT THE INVENTION

### [Mixed Powder]

A mixed powder according to an aspect of the present invention includes a first compound (hereinafter, also referred to as a “first component”) and a second compound (hereinafter, also referred to as a “second component”).

The first compound is a compound containing the fused ring structure having the most rings (hereinafter, also referred to as the “maximum number of rings”) and having three or more rings, in fused ring structures included therein.

The second compound is a compound having 5 or more of the maximum number of rings, and having greater maximum number of rings than the maximum number of rings in the first compound.

When the deposition is conducted using the mixed powder having the above composition, the balance of the vaporization amounts of the first component and the second component can be maintained for a long time. In particular, when materials are continuously heated sequentially to form films on a plurality of substrates, it is possible to form a film with stable component proportion from the initial to the end of the deposition process. As a result, the occurrence of defective products and loss of raw materials can be suppressed to the minimum to increase the yield thereof and to improve the productivity thereof.

The above effects are considered to be due to the following mechanisms. That is, a molecule with larger maximum number of rings tend to have more planarity, and in an aspect of the present invention, when compounds having the specific maximum number of rings are used as the first component and the second component, it is enhancing the effect that a molecule of one component is easily between molecules of the other component. As a result, it becomes easier to obtain a state in which both components are sufficiently mixed at the molecular level. It is consider that when this homogeneous mixing state is achieved, the balance of the vaporization amounts of both components can be maintained for a long time.

The above effects are not intended to mean that a film can be formed with a fixed proportion on all substrates used in a continuous deposition process. Even when using the mixed powder according to an aspect of the present invention, it can be concerned that the component proportion is slightly changed, especially in the final stage of the process, but it means that the time length in which films can be formed with stable proportions can be greatly improved.

Each member in the mixed powder will be described below.

### <First Compound (First Component)>

The first component is a compound containing the fused ring structure having the most rings (the maximum number of rings) and having three or more rings, in fused ring structures included therein.

In the present specification, the fused ring structure indicates a monovalent or divalent or more valent structure formed by removing one or two or more hydrogen atoms from one fused ring compound. Provided that when the first component is composed of only one fused ring compound, the fused ring structure is the fused ring compound itself.

In the present specification, the number of rings indicates the number of single rings constituting one fused ring structure, and for example, a naphthalene structure has two rings, and an anthracene structure has three rings.

In the present specification, the number of rings (the maximum number of rings) of the fused ring structure having the most rings in fused ring structures included in one compound indicates the number of rings of the fused ring structure having the most rings in a compound containing two or more fused ring structures in one molecule. For example, the maximum number of rings is 4 in the following compound.

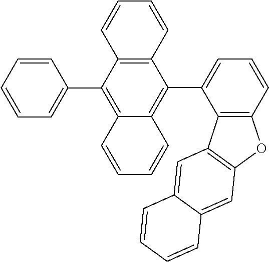 [SMILES: c1ccc(-c2c3ccccc3c(-c3cccc4oc5cc6ccccc6cc5c34)c3ccccc23)cc1]

Fused ring structures (which means the fused ring structure having the maximum number of rings and the other fused ring structures, and which is used with the same meaning below) included in the first component are not particularly limited, and they may be a fused cyclic hydrocarbon structure only containing carbon atoms and hydrogen atoms, or may be a fused cyclic heterocycle structure containing one or two or more heteroatoms. Examples of the heteroatom in the fused cyclic heterocycle structure include O atom, S atom, N atom, B atom, and the like.

The single ring constituting the fused ring structure included in the first component is not particularly limited, and it is generally a four-membered to eight-membered single ring, and for example, a five-membered or six-membered single ring. A plurality of single rings constituting the fused ring structure may be the same as or different from each other.

Examples of the fused ring structure included in the first component include a fused ring structure constituted of one or more six-membered rings and one or more five-membered rings, a fused ring structure constituted of only two or more six-membered rings and the like.

In one embodiment, the first component includes a fused ring structure constituted of only two or more six-membered rings, and may be a compound including only the fused ring structure constituted thereof as the fused ring structure.

In one embodiment, the first component includes a fused ring structure constituted of only two or more six-membered rings, and a fused ring structure constituted of one or more six-membered rings and one or more five-membered rings, and may be a compound including only the fused ring structure constituted thereof as the fused ring structure.

In one embodiment, the first component includes a fused ring structure constituted of one or more six-membered rings and one or more five-membered rings, and may be a compound including only the fused ring structure constituted thereof as the fused ring structure.

The maximum number of rings in the first component is not particularly limited as long as it is 3 or more, and for example, it is 3 or more and 5 or less.

In one embodiment, the maximum number of rings in the first component is 3 or 4.

In the fused ring structure included in the first component, examples of a hydrocarbon structure (fused cyclic hydrocarbon structure) having three rings include an anthracene, a phenanthrene, a phenalene, a fluorene and the like.

In the fused ring structure included in the first component, examples of a heterocycle structure (fused cyclic heterocycle structure) having three rings include a phenanthroline, a phenanthridine, an acridine, a phenazine, a carbazole, a phenoxazine, a phenothiazine, an azacarbazole, a diazacarbazole, a xanthene, dibenzofuran, azadibenzofuran, a diazadibenzofuran, a dibenzothiophene, an azadibenzothiophene, a diazadibenzothiophene and the like.

In the fused ring structure included in the first component, examples of a hydrocarbon structure (fused cyclic hydrocarbon structure) having 4 rings include a benzanthracene, a benzophenanthrene, a pyrene, a chrysene, a triphenylene, a tetracene, a benzofluorene, a fluoranthene, structures represented by the formulas (TEMP-7) to (TEMP-15) defined in the [Definition], and the like.

In the fused ring structure included in the first component, examples of a heterocycle structure (fused cyclic heterocycle structure) having 4 rings include a benzocarbazole, a naphthobenzofuran, an azanaphthobenzofuran, a diazanaphthobenzofuran, a naphthobenzothiophene, an azanaphthobenzothiophene, a diazanaphthobenzothiophene, structures represented by the formulas (TEMP-22) to (TEMP-33) defined in the [Definition], and the like.

In the fused ring structure included in the first component, examples of a hydrocarbon structure (fused cyclic hydrocarbon structure) having 5 rings include a benzochrysene, a benzotriphenylene, a pentacene, a dibenzofluorene, a benzofluoranthene, a perylene, structures represented by the formulas (TEMP-1) to (TEMP-6) defined in the [Definition], and the like.

In the fused ring structure included in the first component, examples of a heterocycle structure (fused cyclic heterocycle structure) having 5 rings include a dibenzocarbazole, a dinaphthofuran, a dinaphthothiophene, structures represented by the formulas (TEMP-16) to (TEMP-21) defined in the [Definition], and the like.

In the fused ring structure included in the second component described later, Examples of a hydrocarbon structure (fused cyclic hydrocarbon structure) having 5 rings and a heterocycle structure (fused cyclic heterocycle structure) having 5 rings include the same as those described above.

The number of the fused ring structure included in the first component is not particularly limited, and for example, it is 1 or more and 5 or less. When a plurality of fused ring structures is included in the first component, the plurality of fused ring structures is the same as or different from each other. It may include two or more fused ring structures having the maximum number of rings in one molecule.

In one embodiment, the number of the fused ring structure included in the first component is 1, 2 or 3.

The fused ring structure included in the first component may have a substituent, and when two or more fused ring structures is included therein, a divalent or more valent group may be included between the fused ring structures.

As the substituent, a group selected from the group consisting of
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇)
(wherein R₉₀₁ to R₉₀₇ are independently
a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted monovalent heterocyclic group having 5 to 50 ring atoms; when two or more of each of R₉₀₁ to R₉₀₇ are present, the two or more of each of R₉₀₁ to R₉₀₇ may be the same as or different from each other),
a halogen atom, a cyano group, a nitro group,
a substituted or unsubstituted aryl group having 6 to 50 ring carbon atoms, and
a substituted or unsubstituted monovalent heterocyclic group having 5 to 50 ring atoms can be given.

In one embodiment, the substituent is a group selected from the group consisting of an alkyl group having 1 to 50 carbon atoms, an aryl group having 6 to 50 ring carbon atoms, and a monovalent heterocyclic group having 5 to 50 ring atoms.

In one embodiment, the substituent is a group selected from the group consisting of an alkyl group having 1 to 18 carbon atoms, an aryl group having 6 to 18 ring carbon atoms, and a monovalent heterocyclic group having 5 to 18 ring atoms.

In one embodiment, the substituent is a group having 5 or less, 4 or less, or 3 or less of maximum number of rings in the above-mentioned groups.

As the divalent or more valent group, a group formed by removing one or more hydrogen atoms from the substituent can be used.

The molecular weight of the first component is not particularly limited, and for example, it is 100 to 1,500. The molecular weight of the first component may be 150 or more, 200 or more, or 300 or more, and it may also be 1,000 or less, 900 or less, or 800 or less.

In one embodiment, the first component is one or more compounds selected from the group consisting of compounds represented by the following formulas (2) and (H11) to (H16).

In one embodiment, the first component is a compound having 3 to 5 of maximum number of rings in the compounds represented by the following formulas (2) and (H11) to (H16).

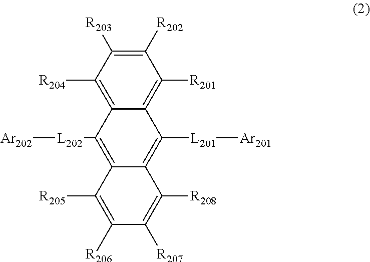

In the formula (2),
R₂₀₁ to R₂₀₈ are independently
a hydrogen atom,
a substituted or unsubstituted alkyl group having 1 to 50 carbon atoms,
a substituted or unsubstituted haloalkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —Si(R₉₀₁) (R₉₀₂)(R₉₀₃),
a group represented by —O—(R₉₀₄),
a group represented by —S—(R₉₀₅),
a group represented by —N(R₉₀₆)(R₉₀₇),
a substituted or unsubstituted aralkyl group having 7 to 50 carbon atoms,
a group represented by —C(═O)R₈₀₁,
a group represented by —COOR₈₀₂,
a halogen atom,
a cyano group,
a nitro group,
a substituted or unsubstituted aryl group having 6 to 50 ring carbon atoms, or
a substituted or unsubstituted heterocyclic group having 5 to 50 ring atoms;
L₂₀₁ and L₂₀₂ are independently
a single bond,
a substituted or unsubstituted arylene group having 6 to 50 ring carbon atoms, or
a substituted or unsubstituted divalent heterocyclic group having 5 to 50 ring atoms;
Ar₂₀₁ and Ar₂₀₂ are independently
a substituted or unsubstituted aryl group having 6 to 50 ring carbon atoms, or
a substituted or unsubstituted heterocyclic group having 5 to 50 ring atoms;
R₉₀₁ to R₉₀₇, R₈₀₁ and R₈₀₂ are independently
a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted heterocyclic group having 5 to 50 ring atoms;
when a plurality of R₉₀₁'s is present, the plurality of R₉₀₁'s is the same as or different from each other;
when a plurality of R₉₀₂'s is present, the plurality of R₉₀₂'s is the same as or different from each other;
when a plurality of R₉₀₃'s is present, the plurality of R₉₀₃'s is the same as or different from each other;
when a plurality of R₉₀₄'s is present, the plurality of R₉₀₄'s is the same as or different from each other;
when a plurality of R₉₀₅'s is present, the plurality of R₉₀₅'s is the same as or different from each other;
when a plurality of R₉₀₆'s is present, the plurality of R₉₀₆'s is the same as or different from each other;
when a plurality of R₉₀₇'s is present, the plurality of R₉₀₇'s is the same as or different from each other;
when a plurality of R₈₀₁'s is present, the plurality of R₈₀₁'s is the same as or different from each other; and
when a plurality of R₈₀₂'s is present, the plurality of R₈₀₂'s is the same as or different from each other.

In one embodiment, L₂₀₁ and L₂₀₂ in the compound represented by the formula (2) are independently a single bond, or a substituted or unsubstituted arylene group having 6 to 50 ring carbon atoms, and Ar₂₀₁ and Ar₂₀₂ are independently a phenyl group, a naphthyl group, a phenanthryl group, a biphenyl group, a terphenyl group, a diphenylfluorenyl group, a dimethylfluorenyl group, a benzodiphenylfluorenyl group, a benzodimethylfluorenyl group, a dibenzofuranyl group, a dibenzothienyl group, a naphthobenzofuranyl group, or a naphthobenzothienyl group.

In one embodiment, the compound represented by the formula (2) is represented by the following formula (201):

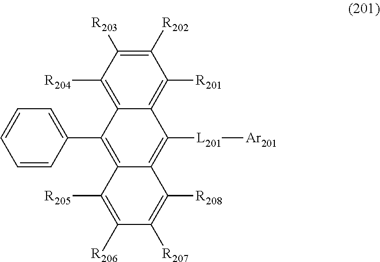

wherein in the formula (201), L₂₀₁ and Ar₂₀₁ are independently the same as defined for L₂₀₁ and Ar₂₀₁ in the formula (2), and R₂₀₁ to R₂₀₈ are independently the same as defined for R₂₀₁ to R₂₀₈ in the formula (2).

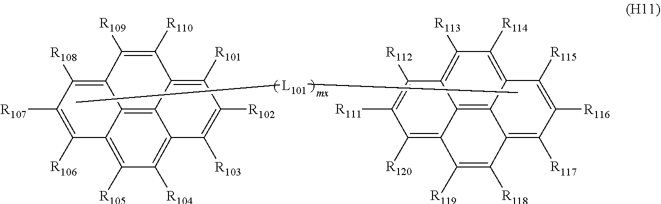

In the formula (H11),
R₁₀₁ to R₁₁₀, and R₁₁₁ to R₁₂₀ are independently
a hydrogen atom,
a substituted or unsubstituted alkyl group having 1 to 50 carbon atoms,
a substituted or unsubstituted haloalkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —Si(R₉₀₁) (R₉₀₂)(R₉₀₃),
a group represented by —O—(R₉₀₄),
a group represented by —S—(R₉₀₅),
a substituted or unsubstituted aralkyl group having 7 to 50 carbon atoms,
a group represented by —C(═O)R₈₀₁,
a group represented by —COOR₈₀₂,
a halogen atom,
a cyano group,
a nitro group,
a substituted or unsubstituted aryl group having 6 to 50 ring carbon atoms, or
a substituted or unsubstituted heterocyclic group having 5 to 50 ring atoms;
provided that one of R₁₀₁ to R₁₁₀ represents a bonding position with L₁₀₁, and one of R₁₁₁ to R₁₂₀ represents a bonding position with L₁₀₁;
L₁₀₁ is
a single bond,
a substituted or unsubstituted arylene group having 6 to 24 ring carbon atoms, or
a substituted or unsubstituted divalent heterocyclic group having 5 to 24 ring atoms;
mx is 0, 1, 2, 3, 4 or 5;
when two or more L₁₀₁'s are present, the two or more L₁₀₁'s are the same as or different from each other;
R₉₀₁ to R₉₀₅, R₈₀₁ and R₈₀₂ are independently the same as defined for R₉₀₁ to R₉₀₅, R₈₀₁ and R₈₀₂ in the formula (2).

In one embodiment, the compound represented by the formula (H11) is a compound represented by the following formula (H111):

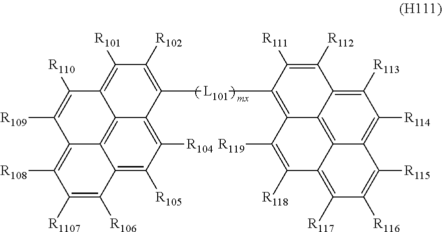

wherein in the formula (H111),
   R₁₀₁, R₁₀₂, R₁₀₄ to R₁₁₀, and R₁₁₁ to R₁₁₉ are independently
   a hydrogen atom,
   a substituted or unsubstituted alkyl group having 1 to 50 carbon atoms,
   a substituted or unsubstituted haloalkyl group having 1 to 50 carbon atoms,
   a substituted or unsubstituted alkenyl group having 2 to 50 carbon atoms,
   a substituted or unsubstituted alkynyl group having 2 to 50 carbon atoms,
   a substituted or unsubstituted cycloalkyl group having 3 to 50 ring carbon atoms,
   a group represented by —Si(R₉₀₁) (R₉₀₂)(R₉₀₃),
   a group represented by —O—(R₉₀₄),
   a group represented by —S—(R₉₀₅),
   a substituted or unsubstituted aralkyl group having 7 to 50 carbon atoms,
   a group represented by —C(═O)R₈₀₁,
   a group represented by —COOR₈₀₂,
   a halogen atom,
   a cyano group,
   a nitro group,
   a substituted or unsubstituted aryl group having 6 to 50 ring carbon atoms, or
   a substituted or unsubstituted heterocyclic group having 5 to 50 ring atoms; and
   L₁₀₁ and mx are independently the same as defined for L₁₀₁ and mx in the formula (H11).

In one embodiment, mx is 1 or 2.

In one embodiment, L₁₀₁ is a substituted or unsubstituted arylene group having 6 to 24 ring carbon atoms.

In one embodiment, the first component is a compound (sometimes referred to as bispirene compound) including only two pyrene rings in the molecule thereof.

In one embodiment, the compound represented by the formula (H11) is a bispirene compound.

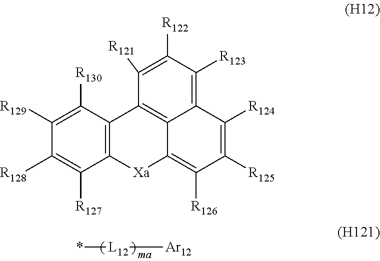

In the formula (H12),
Xa is an oxygen atom, a sulfur atom, C(R₁₂₀₁)(R₁₂₀₂), or Si(R₁₂₀₃)(R₁₂₀₄);
R₁₂₀₁ to R₁₂₀₄ are independently
a hydrogen atom,
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —Si(R₉₀₁) (R₉₀₂)(R₉₀₃),
a group represented by —O—(R₉₀₄),
a group represented by —S—(R₉₀₅),
a group represented by —N(R₉₀₆)(R₉₀₇),
a halogen atom,
a cyano group,
a nitro group,
a substituted or unsubstituted aryl group having 6 to 50 ring carbon atoms, or
a substituted or unsubstituted heterocyclic group having 5 to 50 ring atoms;
one or more sets of the adjacent two or more of R₁₂₁ to R₁₃₀
form a substituted or unsubstituted single ring by bonding with each other,
form a substituted or unsubstituted fused ring by bonding with each other, or
do not bond with each other;
R₁₂₁ to R₁₃₀ which do not form the substituted or unsubstituted single ring and which do not form the substituted or unsubstituted fused ring are independently
a hydrogen atom,
a substituted or unsubstituted alkyl group having 1 to 50 carbon atoms,
a substituted or unsubstituted haloalkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —Si(R₉₀₁) (R₉₀₂)(R₉₀₃),
a group represented by —O—(R₉₀₄),
a group represented by —S—(R₉₀₅),
a group represented by —N(R₉₀₆)(R₉₀₇),
a substituted or unsubstituted aralkyl group having 7 to 50 carbon atoms,
a group represented by —C(═O)R₈₀₁,
a group represented by —COOR₈₀₂,
a halogen atom,
a nitro group,
a substituted or unsubstituted aryl group having 6 to 50 ring carbon atoms,
a substituted or unsubstituted heterocyclic group having 5 to 50 ring atoms, or
a group represented by the formula (H121);
provided that at least one of R₁₂₁ to R₁₃₀ is the group represented by the formula (H121);
when a plurality of groups represented by the formula (H121) is present, the plurality of groups represented by the formula (H121) is the same as or different from each other;
L₁₂ is
a single bond,
a substituted or unsubstituted arylene group having 6 to 50 ring carbon atoms, or
a substituted or unsubstituted divalent heterocyclic group having 5 to 50 ring atoms;
ma is 0, 1, 2 or 3;
when two or more L₁₂'s are present, the two or more L₁₂'s are the same as or different from each other;
Ar₁₂ is a substituted or unsubstituted aryl group having 6 to 50 ring carbon atoms, or a substituted or unsubstituted heterocyclic group having 5 to 50 ring atoms;
when two or more Ar₁₂'s are present, the two or more Ar₁₂'s are the same as or different from each other;
in the formula (H121) represents a bonding position; and
R₉₀₁ to R₉₀₇, R₈₀₁ and R₈₀₂ are independently the same as defined for R₉₀₁ to R₉₀₇, R₈₀₁ and R₈₀₂ in the formula (2).

In one embodiment, L₁₂ is a single bond, a substituted or unsubstituted arylene group having 6 to 15 ring carbon atoms, or a substituted or unsubstituted divalent heterocyclic group having 5 to 15 ring atoms.

In one embodiment, Ar₁₂ is a substituted or unsubstituted aryl group including four or more rings, or a substituted or unsubstituted heterocyclic group including four or more rings.

In one embodiment, Ar₁₂ is a substituted or unsubstituted aryl group including four or more rings. In one embodiment, R₁₂₉ is the group represented by the formula (H121).

In one embodiment, Xa is an oxygen atom.

In one embodiment, the compound represented by the formula (H12) is a compound represented by the following formula (H122).

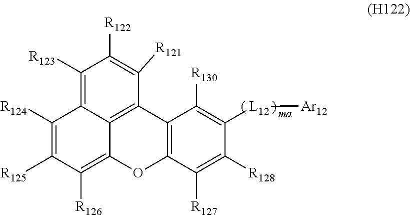

In the formula (H122),
R₁₂₁ to R₁₂₈ and R₁₃₀ are independently
a hydrogen atom,
a substituted or unsubstituted alkyl group having 1 to 50 carbon atoms,
a substituted or unsubstituted haloalkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —Si(R₉₀₁) (R₉₀₂)(R₉₀₃),
a group represented by —O—(R₉₀₄),
a group represented by —S—(R₉₀₅),
a group represented by —N(R₉₀₆)(R₉₀₇),
a substituted or unsubstituted aralkyl group having 7 to 50 carbon atoms,
a group represented by —C(═O)R₈₀₁,
a group represented by —COOR₈₀₂,
a halogen atom,
a nitro group,
a substituted or unsubstituted aryl group having 6 to 50 ring carbon atoms, or
a substituted or unsubstituted heterocyclic group having 5 to 50 ring atoms; and
Ar₁₂, L₁₂ and ma are independently the same as defined for Ar₁₂, L₁₂ and ma in the formula (H121).

In one embodiment, ma is 1 or 2.

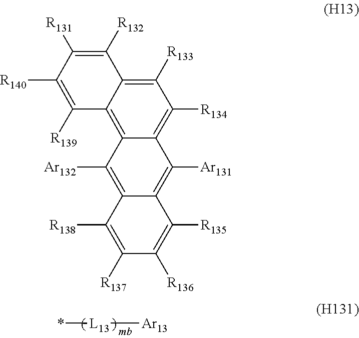

In the formula (H13),
R₁₃₁ to R₁₄₀, Ar₁₃₁ and Ar₁₃₂ are independently
a hydrogen atom,
a substituted or unsubstituted alkyl group having 1 to 50 carbon atoms,
a substituted or unsubstituted haloalkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —Si(R₉₀₁) (R₉₀₂)(R₉₀₃),
a group represented by —O—(R₉₀₄),
a group represented by —S—(R₉₀₅),
a substituted or unsubstituted aralkyl group having 7 to 50 carbon atoms,
a group represented by —C(═O)R₈₀₁,
a group represented by —COOR₈₀₂,
a halogen atom,
a cyano group,
a nitro group,
a substituted or unsubstituted aryl group having 6 to 50 ring carbon atoms,
a substituted or unsubstituted heterocyclic group having 5 to 50 ring atoms, or
a group represented by the formula (H131);
provided that at least one of R₁₃₁ to R₁₄₀, Ar₁₃₁ and Ar₁₃₂ is the group represented by the formula (H131);
when a plurality of groups represented by the formula (H131) is present, the plurality of groups represented by the formula (H131) is the same as or different from each other;
L₁₃ is
a single bond,
a substituted or unsubstituted arylene group having 6 to 50 ring carbon atoms, or
a substituted or unsubstituted divalent heterocyclic group having 5 to 50 ring atoms;
Ar₁₃ is
a substituted or unsubstituted aryl group having 6 to 50 ring carbon atoms, or
a substituted or unsubstituted heterocyclic group having 5 to 50 ring atoms;
mb is 0, 1, 2, 3, 4 or 5;
when two or more L₁₃'s are present, the two or more L₁₃'s are the same as or different from each other;
when two or more Ar₁₃'s are present, the two or more Ar₁₃'s are the same as or different from each other;
* in the formula (H131) represents a bonding position with the benz[a]anthracene ring in the formula (H13); and
R₉₀₁ to R₉₀₅, R₈₀₁ and R₈₀₂ are independently the same as defined for R₉₀₁ to R₉₀₅, R₈₀₁ and R₈₀₂ in the formula (2).

In one embodiment, at least one of Ar₁₃₁ and Ar₁₃₂ is the group represented by the formula (H131).

In one embodiment, the compound represented by the formula (H13) is a compound represented by the following formula (H132) or (H133):

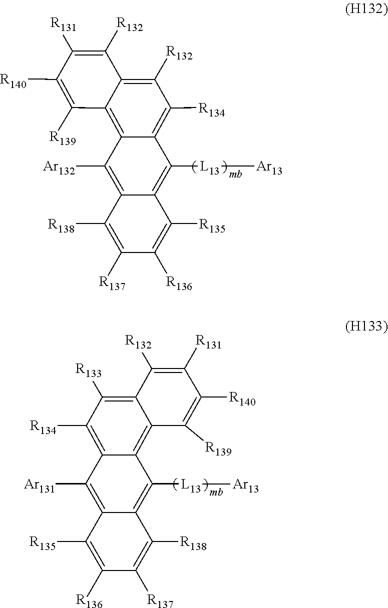

wherein in the formulas (H132) and (H133),
R₁₃₁ to R₁₄₀, Ar₁₃₁ and Ar₁₃₂ are independently
a hydrogen atom,
a substituted or unsubstituted alkyl group having 1 to 50 carbon atoms,
a substituted or unsubstituted haloalkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —Si(R₉₀₁) (R₉₀₂)(R₉₀₃),
a group represented by —O—(R₉₀₄),
a group represented by —S—(R₉₀₅),
a substituted or unsubstituted aralkyl group having 7 to 50 carbon atoms,
a group represented by —C(═O)R₈₀₁,
a group represented by —COOR₈₀₂,
a halogen atom,
a cyano group,
a nitro group,
a substituted or unsubstituted aryl group having 6 to 50 ring carbon atoms, or
a substituted or unsubstituted heterocyclic group having 5 to 50 ring atoms; and
L₁₃, Ar₁₃ and mb are independently the same as defined for L₁₃, Ar₁₃ and mb in the formula (H131).

In one embodiment, mb is 0, 1 or 2.

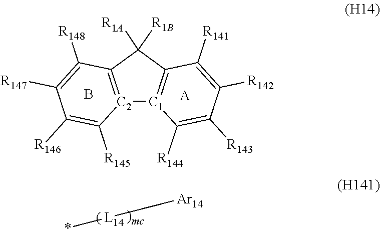

In the formula (H14),
R_{1A} and R_{1B} are independently
a substituted or unsubstituted alkyl group having 1 to 15 carbon atoms,
a substituted or unsubstituted aryl group having 6 to 17 ring carbon atoms, or
a substituted or unsubstituted heterocyclic group having 5 to 17 ring atoms;
provided that at least one of R_{1A} and R_{1B} is a substituted or unsubstituted alkyl group having 1 to 15 carbon atoms;
any one set of the adjacent two or more of R₁₄₁ to R₁₄₄ and the adjacent two or more of R₁₄₅ to R₁₄₈
forms a substituted or unsubstituted single ring by bonding with each other, or
forms a substituted or unsubstituted fused ring by bonding with each other;
a group represented by the formula (H141)
is bonded with the carbon atom with which R₁₄₂ is bonded, or a carbon atom which is farthest from the carbon atom C₁ in the ring A being bonded with the carbon atom C₂ in the ring B via a single bond, of carbon atoms constituting the single ring on the ring A side and the fused ring on the ring A side, in the case where the substituted or unsubstituted single ring or the substituted or unsubstituted fused ring is formed on the ring A side, or
is bonded with the carbon atom with which R₁₄₂ is bonded in the case where the substituted or unsubstituted single ring or the substituted or unsubstituted fused ring is not formed on the ring A side, but is formed on the ring B side;
R₁₄₂ which are not the group represented by the formula (H141), and R₁₄₁, R₁₄₃, R₁₄₄ and R₁₄₅ to R₁₄₈ which do not form the substituted or unsubstituted single ring and which do not form the substituted or unsubstituted fused ring are independently
a hydrogen atom,
a substituted or unsubstituted alkyl group having 1 to 50 carbon atoms,
a substituted or unsubstituted haloalkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —Si(R₉₀₁) (R₉₀₂)(R₉₀₃),
a group represented by —O—(R₉₀₄),
a group represented by —S—(R₉₀₅),
a group represented by —N(R₉₀₆)(R₉₀₇),
a substituted or unsubstituted aralkyl group having 7 to 50 carbon atoms,
a group represented by —C(═O)R₈₀₁,
a group represented by —COOR₈₀₂,
a halogen atom,
a cyano group,
a nitro group,
a substituted or unsubstituted aryl group having 6 to 17 ring carbon atoms, or
a substituted or unsubstituted heterocyclic group having 5 to 17 ring atoms;
in the formula (H141),
Ar₁₄ is a substituted or unsubstituted aryl group in which four or more rings are fused, or a substituted or unsubstituted heterocyclic group in which four or more rings are fused;
L₁₄ is
a single bond,
a substituted or unsubstituted arylene group having 6 to 17 ring carbon atoms, or
a substituted or unsubstituted divalent heterocyclic group having 5 to 17 ring atoms;
mc is 0, 1 or 2;
* represents a bonding position with an atom constituting the ring of the formula (H14);
provided that the compound represented by the formula (H14) does not contain three or more groups of a substituted or unsubstituted aryl group in which four or more rings are fused, and a substituted or unsubstituted heterocyclic group in which four or more rings are fused in the molecule of the compound represented by the formula (H14); and
R₉₀₁ to R₉₀₇, R₈₀₁ and R₈₀₂ are independently the same as defined for R₉₀₁ to R₉₀₇, R₈₀₁ and R₈₀₂ in the formula (2).

In one embodiment, R₁₄₂ is the group represented by the formula (H141).

In one embodiment, R₉₀₁, R₉₀₂, R₉₀₃, R₉₀₄, R₉₀₅, R₉₀₆, R₉₀₇, R₈₀₁ and R₈₀₂ in the compound represented by the formula (H14) are independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 17 ring carbon atoms, or a substituted or unsubstituted heterocyclic group having 5 to 17 ring atoms.

In one embodiment, the compound represented by the formula (H14) is a compound represented by the following formula (H142), (H143) or (H144):

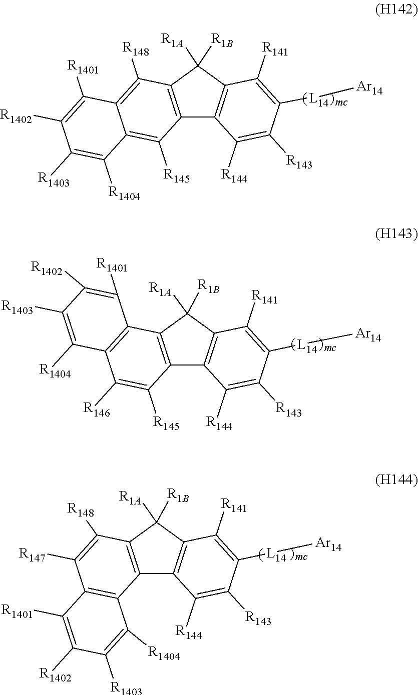

wherein in the formula (H142), (H143) or (H144),
R_{1A}, R_{1B}, R₁₄₁, R₁₄₃, R₁₄₄, R₁₄₅, R₁₄₆, R₁₄₇ and R₁₄₈ are independently the same as defined for R_{1A}, R_{1B}, R₁₄₁, R₁₄₃, R₁₄₄, R₁₄₅, R₁₄₆, R₁₄₇ and R₁₄₈ in the formula (H14);
Ar₁₄, L₁₄ and mc are independently the same as defined for Ar₁₄, L₁₄ and mc in the formula (H141);
all of one or more sets of the adjacent two or more of R₁₄₀₁ to R₁₄₀₄ do not bond with each other; and
R₁₄₀₁ to R₁₄₀₄ are independently
a hydrogen atom,
a substituted or unsubstituted alkyl group having 1 to 50 carbon atoms,
a substituted or unsubstituted haloalkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —Si(R₉₀₁)(R₉₀₂)(R₉₀₃),
a group represented by —O—(R₉₀₄),
a group represented by —S—(R₉₀₅),
a group represented by —N(R₉₀₆)(R₉₀₇),
a substituted or unsubstituted aralkyl group having 7 to 50 carbon atoms,
a group represented by —C(═O)R₈₀₁,
a group represented by —COOR₈₀₂,
a halogen atom,
a cyano group,
a nitro group,
a substituted or unsubstituted aryl group having 6 to 17 ring carbon atoms, or
a substituted or unsubstituted heterocyclic group having 5 to 17 ring atoms.

In one embodiment, mc is 0, 1 or 2.

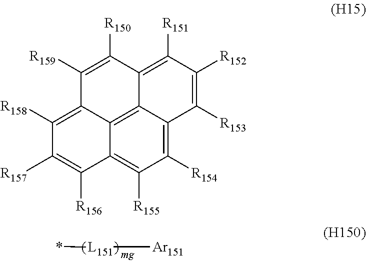

In the formula (H15),
R₁₅₀ to R₁₅₉ are independently
a hydrogen atom,
a substituted or unsubstituted alkyl group having 1 to 50 carbon atoms,
a substituted or unsubstituted haloalkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —Si(R₉₀₁) (R₉₀₂)(R₉₀₃),
a group represented by —O—(R₉₀₄),
a group represented by —S—(R₉₀₅),
a substituted or unsubstituted aralkyl group having 7 to 50 carbon atoms,
a group represented by —C(═O)R₈₀₁,
a group represented by —COOR₈₀₂,
a halogen atom,
a cyano group,
a nitro group,
a substituted or unsubstituted aryl group having 6 to 50 ring carbon atoms,
a substituted or unsubstituted heterocyclic group having 5 to 50 ring atoms, or
a group represented by the formula (H150);
provided that at least one of R₁₅₀ to R₁₅₉ is the group represented by the formula (H150);
when a plurality of groups represented by the formula (H150) is present, the plurality of groups represented by the formula (H150) is the same as or different from each other;
L₁₅₁ is
a single bond,
a substituted or unsubstituted arylene group having 6 to 50 ring carbon atoms, or
a substituted or unsubstituted divalent heterocyclic group having 5 to 50 ring atoms;
Ar₁₅₁ is
a substituted or unsubstituted aryl group having 6 to 50 ring carbon atoms, or
a substituted or unsubstituted heterocyclic group having 5 to 50 ring atoms;
mg is 0, 1, 2, 3, 4 or 5;
when two or more L₁₅₁'s are present, the two or more L₁₅₁'s are the same as or different from each other;
when two or more Ar₁₅₁'s are present, the two or more Ar₁₅₁'s are the same as or different from each other;
* in the formula (H150) represents a bonding position with the pyrene ring in the formula (H15); and
R₉₀₁ to R₉₀₅, R₈₀₁ and R₈₀₂ are independently the same as defined for R₉₀₁ to R₉₀₅, R₈₀₁ and R₈₀₂ in the formula (2).

In one embodiment, R₁₅₃ in the compound represented by the formula (H15) is the group represented by the formula (H150).

In one embodiment, L₁₅₁ is a single bond, or a substituted or unsubstituted arylene group having 6 to 50 ring carbon atoms, and Ar₁₅₁ is a substituted or unsubstituted aryl group having 6 to 50 ring carbon atoms.

In one embodiment, L₁₅₁ is a single bond, or a substituted or unsubstituted arylene group having 6 to 14 ring carbon atoms, and Ar₁₅₁ is a substituted or unsubstituted aryl group having 6 to 14 ring carbon atoms.

In one embodiment, the group represented by the formula (H150) is a group represented by the following formula (H151):

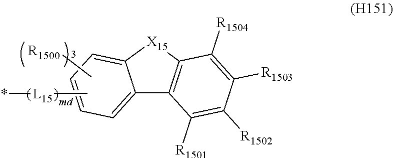

In the formula (H151),
X₁₅ is an oxygen atom, or a sulfur atom;
L₁₅ is
a single bond,
a substituted or unsubstituted arylene group having 6 to 50 ring carbon atoms, or
a substituted or unsubstituted divalent heterocyclic group having 5 to 50 ring atoms;
md is 0, 1, 2, 3, 4 or 5;
when two or more L₁₅'s are present, the two or more L₁₅'s are the same as or different from each other;
one or more sets of the adjacent two or more of R₁₅₀₀ to R₁₅₀₄
form a substituted or unsubstituted single ring by bonding with each other,
form a substituted or unsubstituted fused ring by bonding with each other, or
do not bond with each other;
R₁₅₀₀ to R₁₅₀₄ which do not form the substituted or unsubstituted single ring and which do not form the substituted or unsubstituted fused ring are independently
a hydrogen atom,
a substituted or unsubstituted alkyl group having 1 to 50 carbon atoms,
a substituted or unsubstituted haloalkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —Si(R₉₀₁) (R₉₀₂)(R₉₀₃),
a group represented by —O—(R₉₀₄),
a group represented by —S—(R₉₀₅),
a substituted or unsubstituted aralkyl group having 7 to 50 carbon atoms,
a group represented by —C(═O)R₈₀₁,
a group represented by —COOR₈₀₂,
a halogen atom,
a cyano group,
a nitro group,
a substituted or unsubstituted aryl group having 6 to 50 ring carbon atoms, or
a substituted or unsubstituted heterocyclic group having 5 to 50 ring atoms;
a plurality of R₁₅₀₀'s is the same as or different from each other:
when a plurality of groups represented by the formula (H151) is present, the plurality of groups represented by the formula (H151) is the same as or different from each other; and
* in the formula (H151) represents a bonding position with the pyrene ring in the formula (H15).

In one embodiment, the compound represented by the formula (H15) is a compound represented by the following formula (H152). When R₁₅₃ in the compound represented by the formula (H15) is the group represented by the formula (H151), it is represented by the following formula (H152):

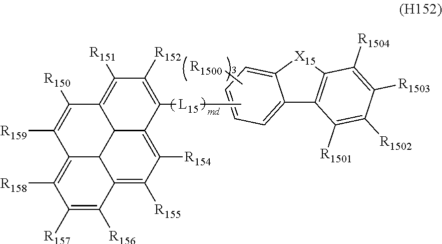

wherein in the formula (H152),
R₁₅₀ to R₁₅₂ and R₁₅₄ to R₁₅₉ are independently
a hydrogen atom,
a substituted or unsubstituted alkyl group having 1 to 50 carbon atoms,
a substituted or unsubstituted haloalkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —Si(R₉₀₁) (R₉₀₂)(R₉₀₃),
a group represented by —O—(R₉₀₄),
a group represented by —S—(R₉₀₅),
a substituted or unsubstituted aralkyl group having 7 to 50 carbon atoms,
a group represented by —C(═O)R₈₀₁,
a group represented by —COOR₈₀₂,
a halogen atom,
a cyano group,
a nitro group,
a substituted or unsubstituted aryl group having 6 to 50 ring carbon atoms, or
a substituted or unsubstituted heterocyclic group having 5 to 50 ring atoms;
X₁₅, L₁₅ and md are independently the same as defined for X₁₅, L₁₅ and md in the formula (H151); and
R₁₅₀₀ to R₁₅₀₄ are independently the same as defined for R₁₅₀₀ to R₁₅₀₄ in the formula (H151).

In one embodiment, md is 0, 1 or 2.

In one embodiment, when md is 0, the compound represented by the formula (H152) is represented by the following formula (H153):

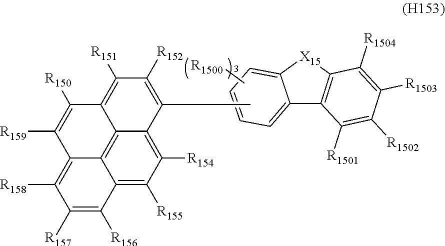

wherein in the formula (H153),

R₁₅₀ to R₁₅₂, R₁₅₄ to R₁₅₉, R₁₅₀₀ to R₁₅₀₄, and X₁₅ are independently the same as defined for R₁₅₀ to R₁₅₂, R₁₅₄ to R₁₅₉, R₁₅₀₀ to R₁₅₀₄, and X₁₅ in the formula (H152).

In one embodiment, the first component is a compound (sometimes referred to as monopirene compound) including only one pyrene ring in the molecule thereof.

In one embodiment, the compound represented by the formula (H15) is a monopirene compound.

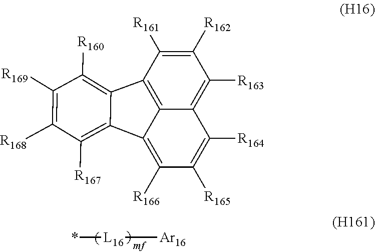

In the formula (H16),
one or more sets of the adjacent two or more of R₁₆₀ to R₁₆₉
form a substituted or unsubstituted single ring by bonding with each other,
form a substituted or unsubstituted fused ring by bonding with each other, or
do not bond with each other;
R₁₆₀ to R₁₆₉ which do not form the substituted or unsubstituted single ring and which do not form the substituted or unsubstituted fused ring are independently
a hydrogen atom,
a substituted or unsubstituted alkyl group having 1 to 50 carbon atoms,
a substituted or unsubstituted haloalkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —Si(R₉₀₁) (R₉₀₂)(R₉₀₃),
a group represented by —O—(R₉₀₄),
a group represented by —S—(R₉₀₅),
a substituted or unsubstituted aralkyl group having 7 to 50 carbon atoms,
a group represented by —C(═O)R₈₀₁,
a group represented by —COOR₈₀₂,
a halogen atom,
a cyano group,
a nitro group,
a substituted or unsubstituted aryl group having 6 to 50 ring carbon atoms,
a substituted or unsubstituted heterocyclic group having 5 to 50 ring atoms, or
a group represented by the formula (H161);
provided that at least one of the substituent in the case where the substituted or unsubstituted single ring has the substituent, the substituent in the case where the substituted or unsubstituted fused ring has the substituent, and R₁₆₀ to R₁₆₉ is the group represented by the formula (H161);
when a plurality of groups represented by the formula (H161) is present, the plurality of groups represented by the formula (H161) is the same as or different from each other;
L₁₆ is
a single bond,
a substituted or unsubstituted arylene group having 6 to 50 ring carbon atoms, or
a substituted or unsubstituted divalent heterocyclic group having 5 to 50 ring atoms;
Ar₁₆ is
a substituted or unsubstituted aryl group having 6 to 50 ring carbon atoms, or
a substituted or unsubstituted heterocyclic group having 5 to 50 ring atoms;
mf is 0, 1, 2, 3, 4 or 5;
when two or more L₁₆'s are present, the two or more L₁₆'s are the same as or different from each other;
when two or more Ar₁₆'s are present, the two or more Ar₁₆'s are the same as or different from each other;
* in the formula (H161) represents a bonding position with the ring represented by the formula (H16); and
R₉₀₁ to R₉₀₅, R₈₀₁ and R₈₀₂ are independently the same as defined for R₉₀₁ to R₉₀₅, R₈₀₁ and R₈₀₂ in the formula (2).

In one embodiment, the first component is a compound represented by the following formula (H162):

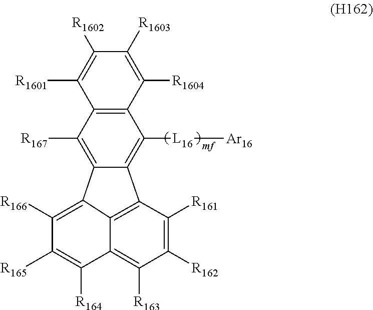

wherein in the formula (H162),
R₁₆₁ to R₁₆₇ and R₁₆₀₁ to R₁₆₀₄ are independently
a hydrogen atom,
a substituted or unsubstituted alkyl group having 1 to 50 carbon atoms,
a substituted or unsubstituted haloalkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —Si(R₉₀₁) (R₉₀₂)(R₉₀₃),
a group represented by —O—(R₉₀₄),
a group represented by —S—(R₉₀₅),
a substituted or unsubstituted aralkyl group having 7 to 50 carbon atoms,
a group represented by —C(═O)R₈₀₁,
a group represented by —COOR₈₀₂,
a halogen atom,
a cyano group,
a nitro group,
a substituted or unsubstituted aryl group having 6 to 50 ring carbon atoms, or
a substituted or unsubstituted heterocyclic group having 5 to 50 ring atoms; and
Ar₁₆, L₁₆ and mf are independently the same as defined for Ar₁₆, L₁₆ and mf in the formula (H16).

In one embodiment, mf is 0, 1 or 2.

Specific examples of the first component include the following compounds. Here, the present invention is not limited to these specific examples. In the present specification, “D” represents a deuterium atom, “Me” represents a methyl group, “tBu” represents a tert-butyl group, and “Ph” represents a phenyl group, in specific examples of the compound.

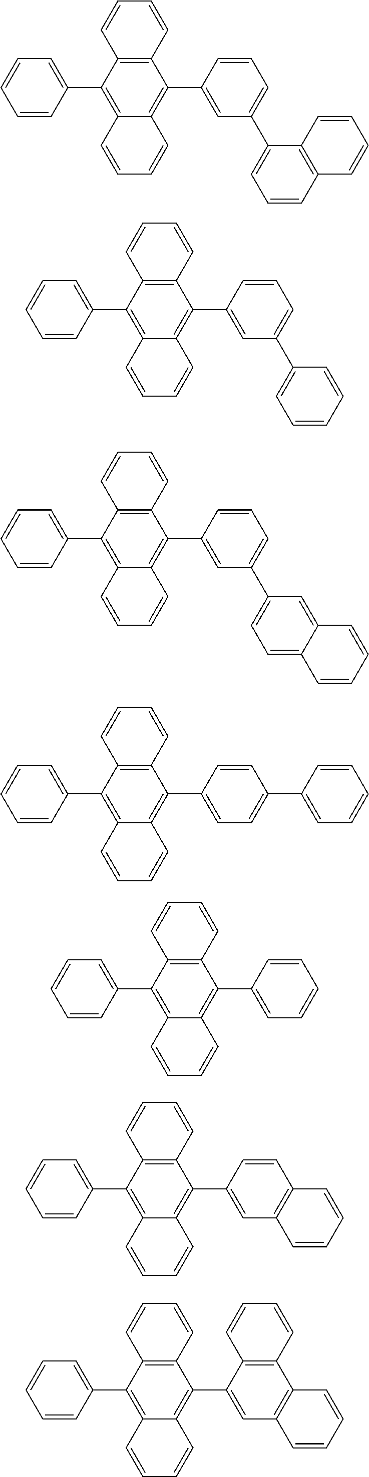

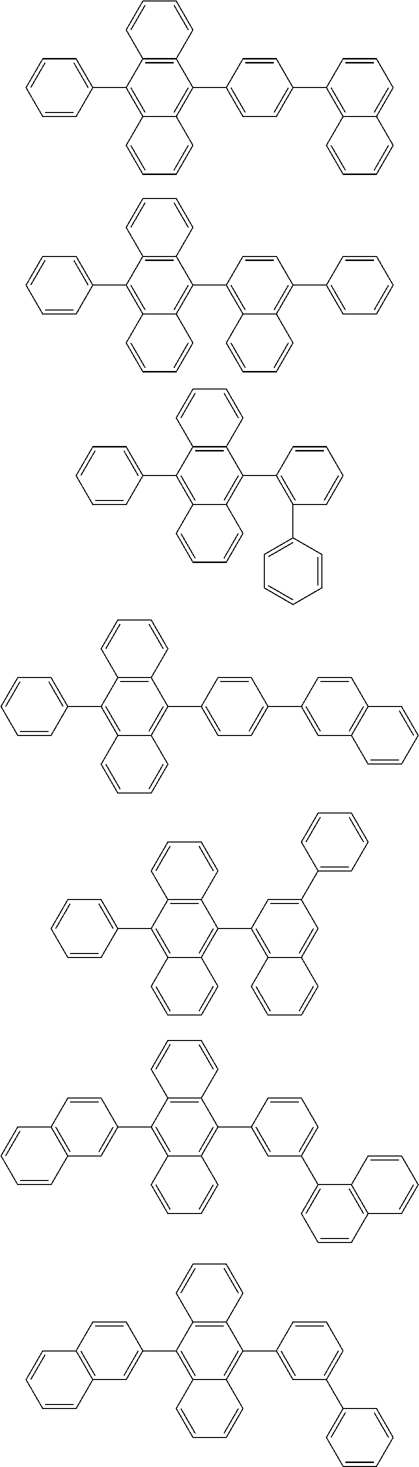

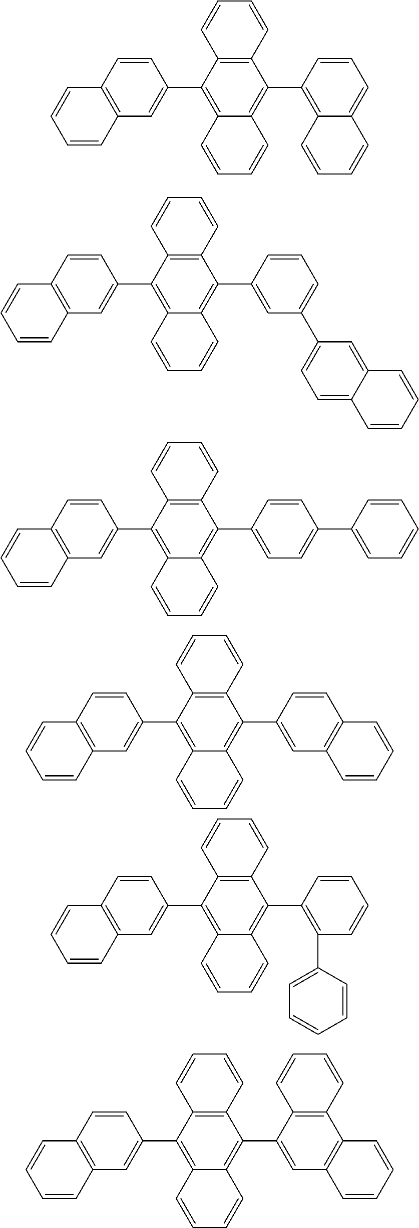

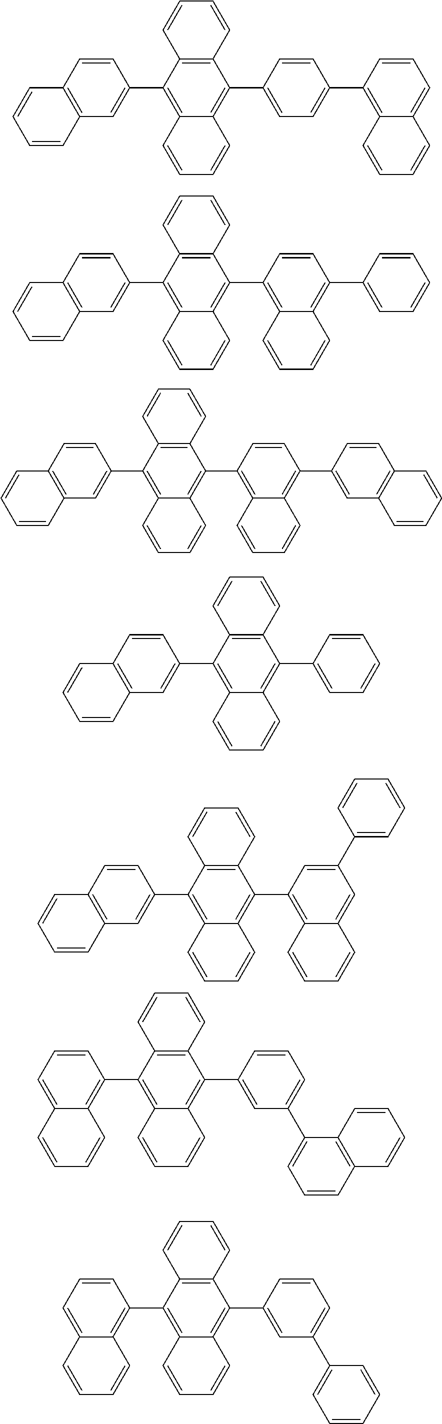

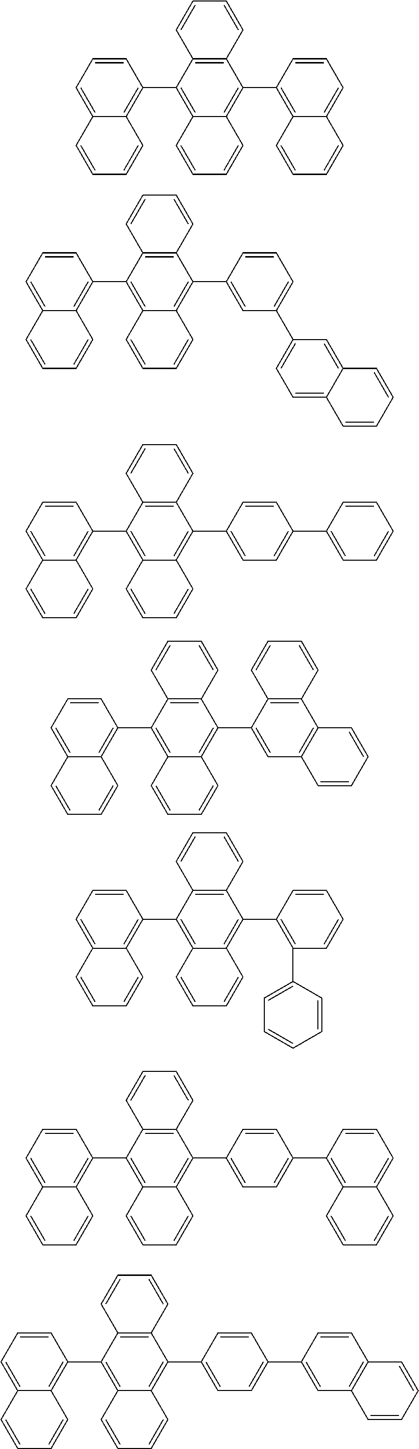

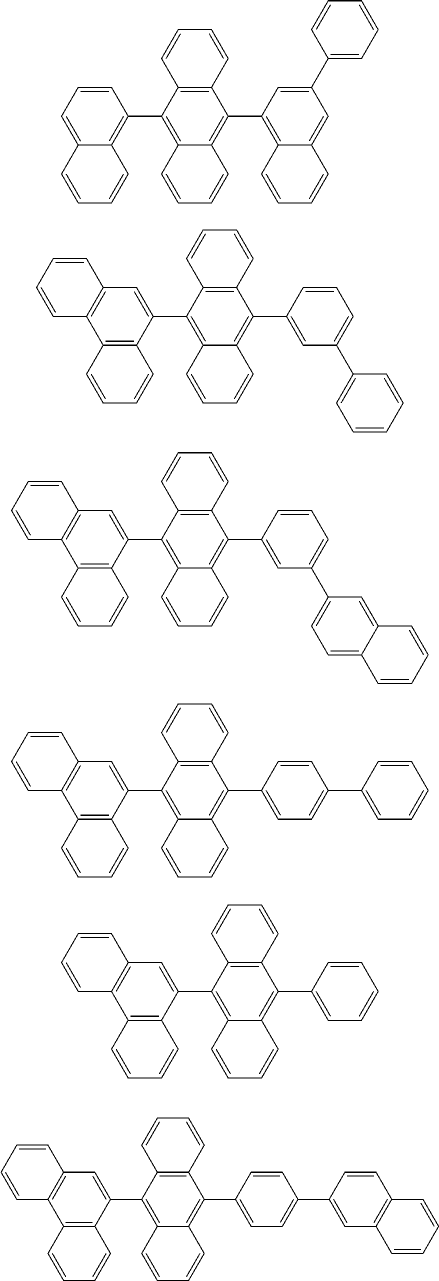

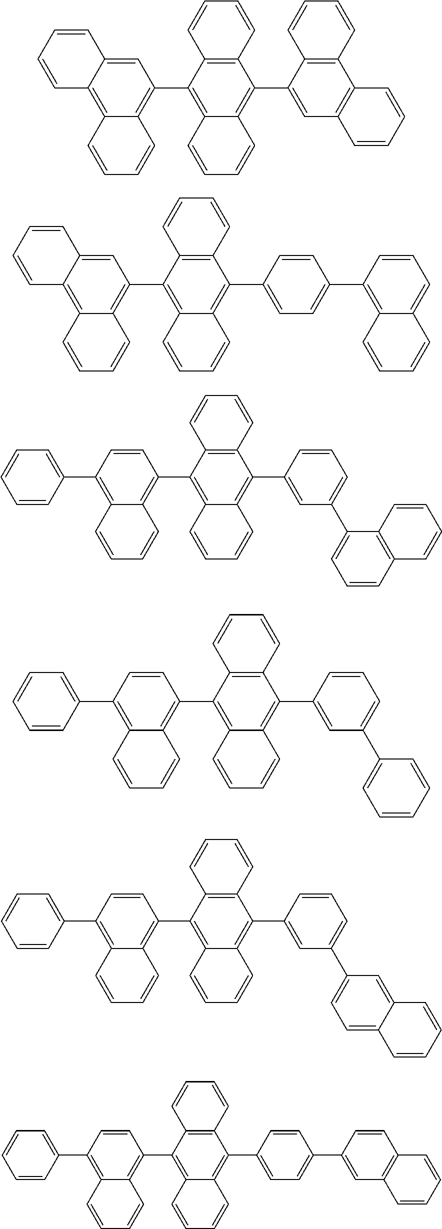

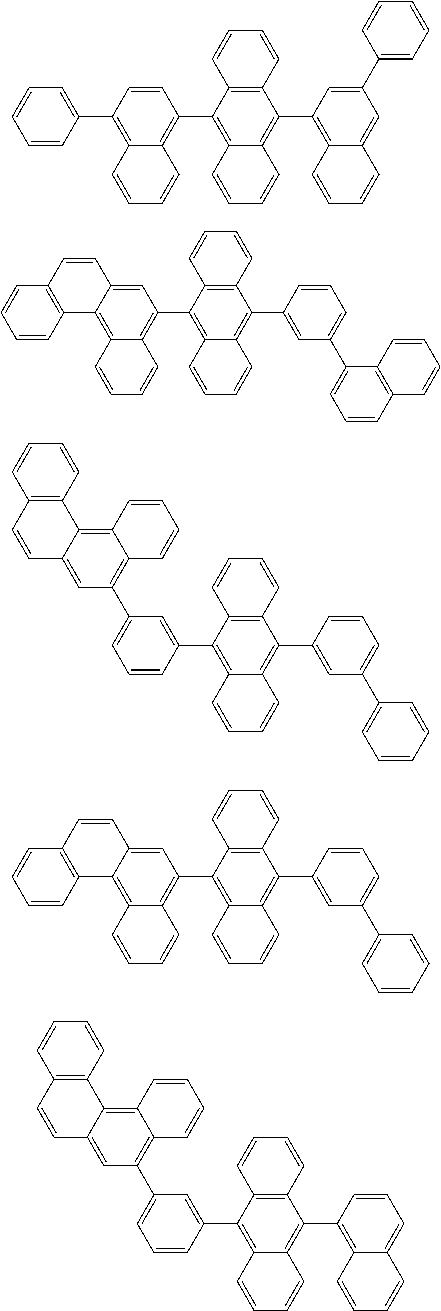

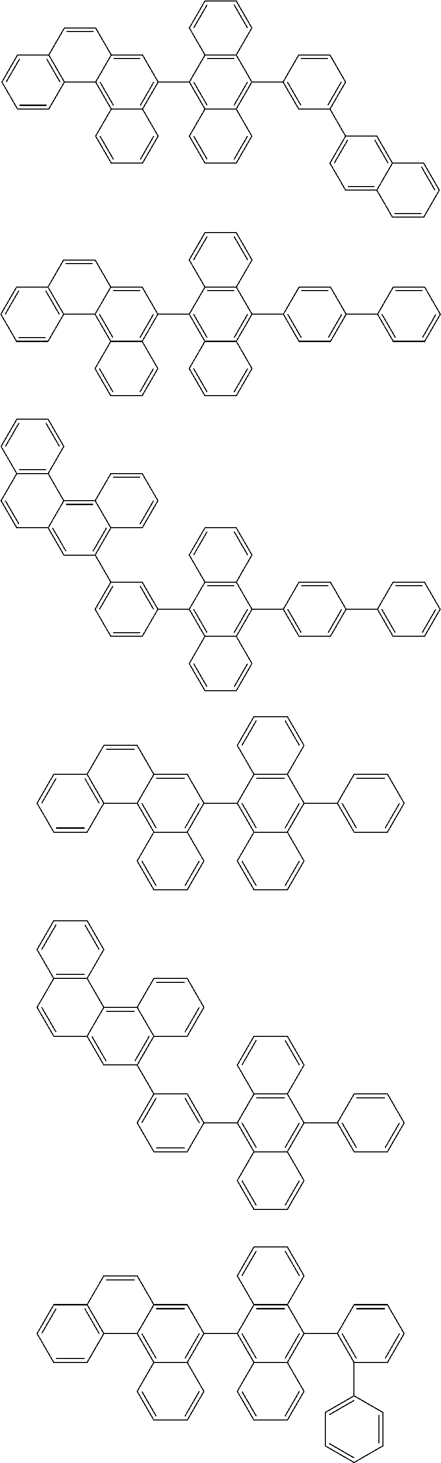

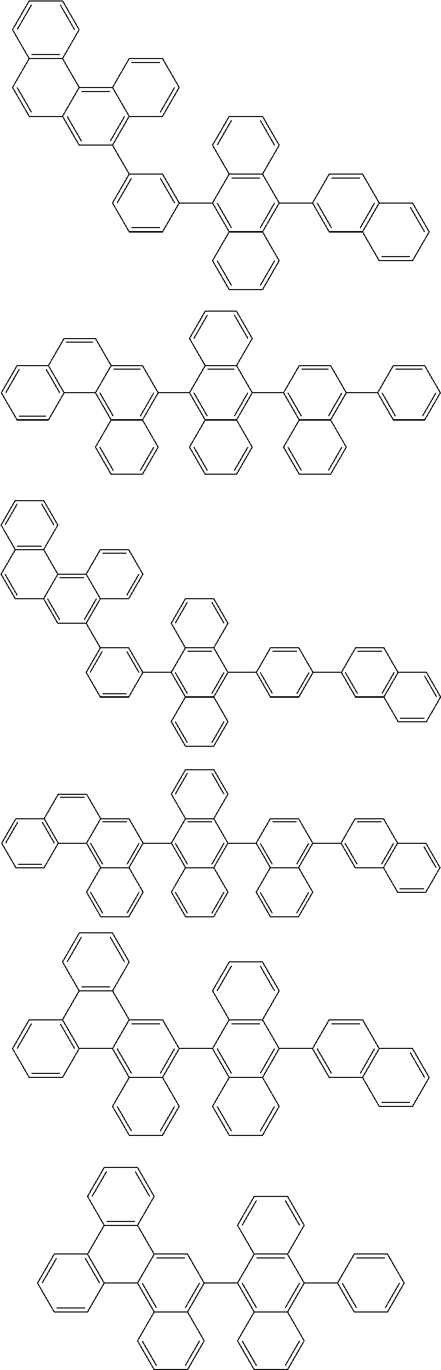

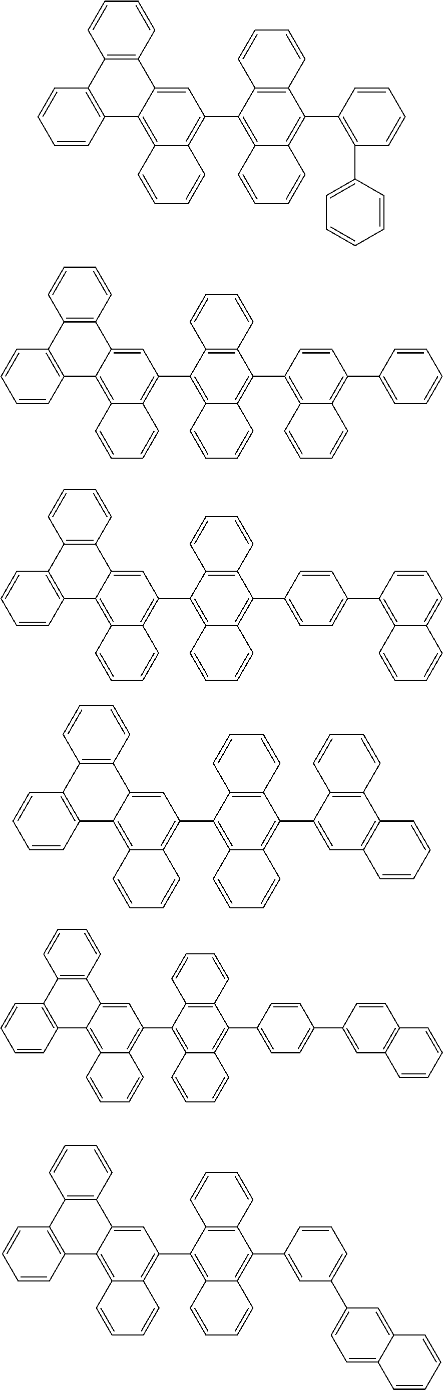

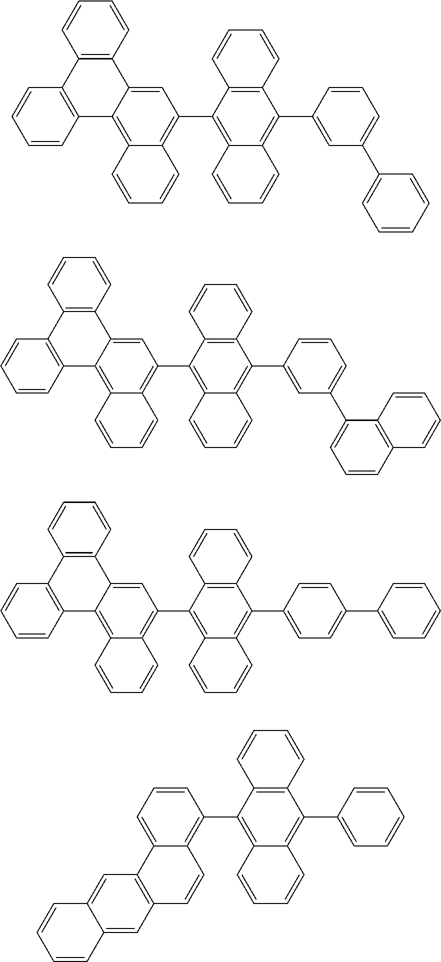

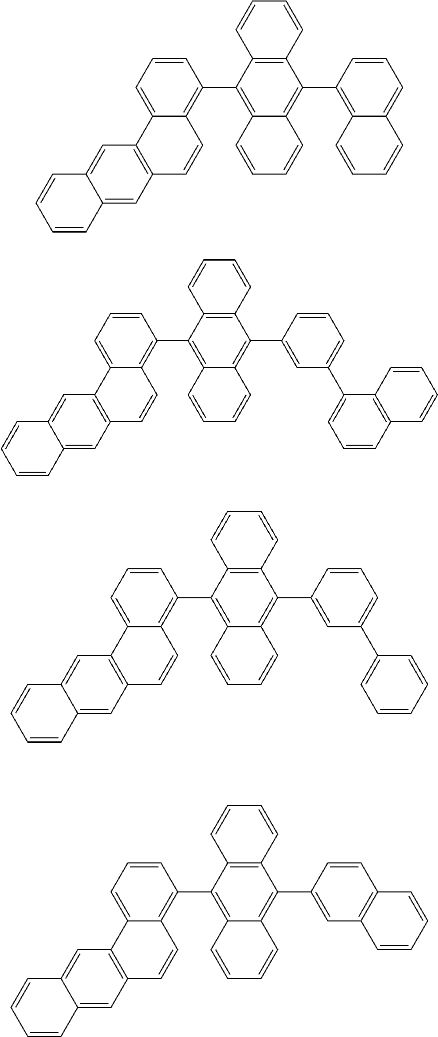

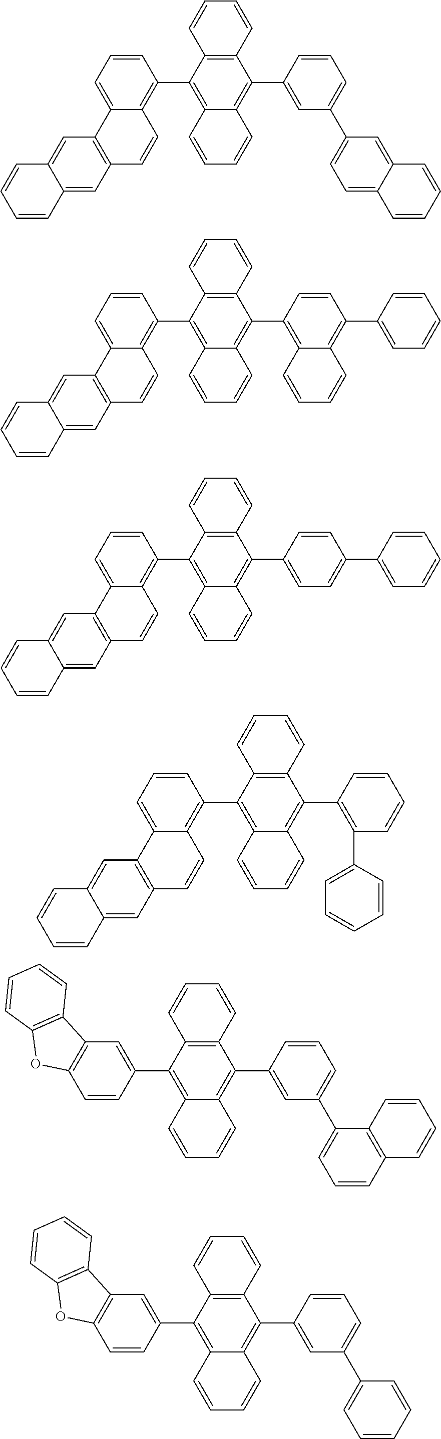

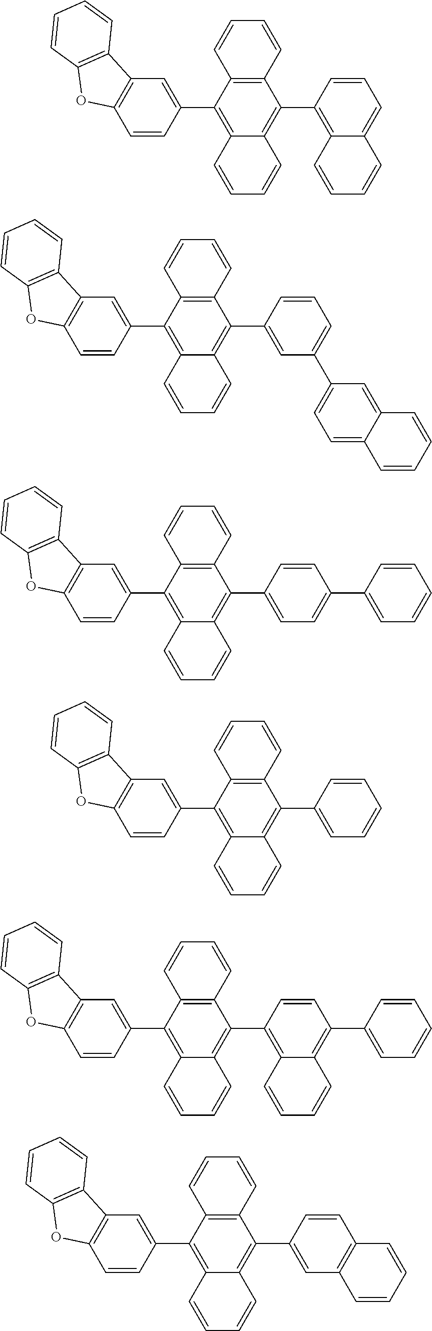

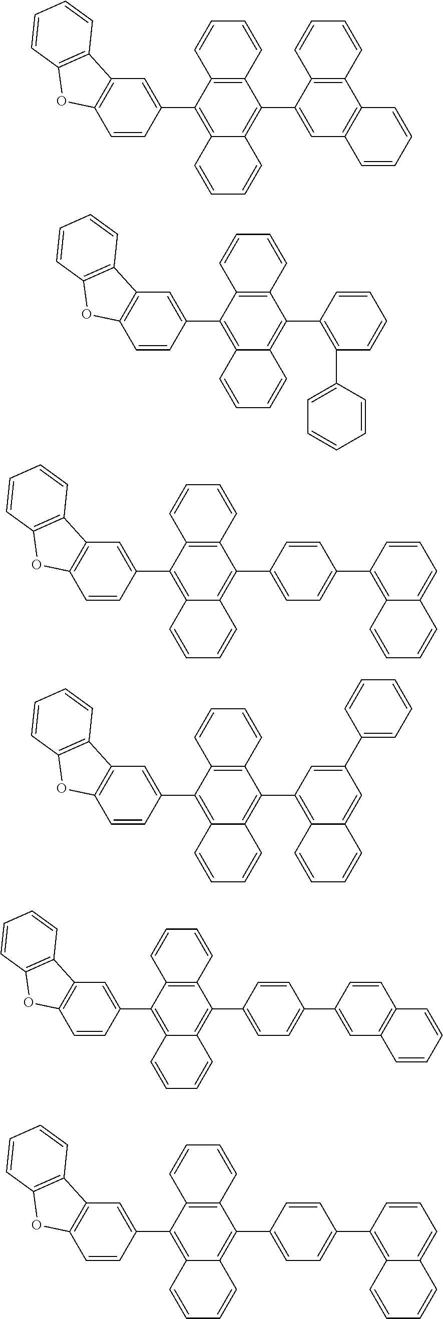

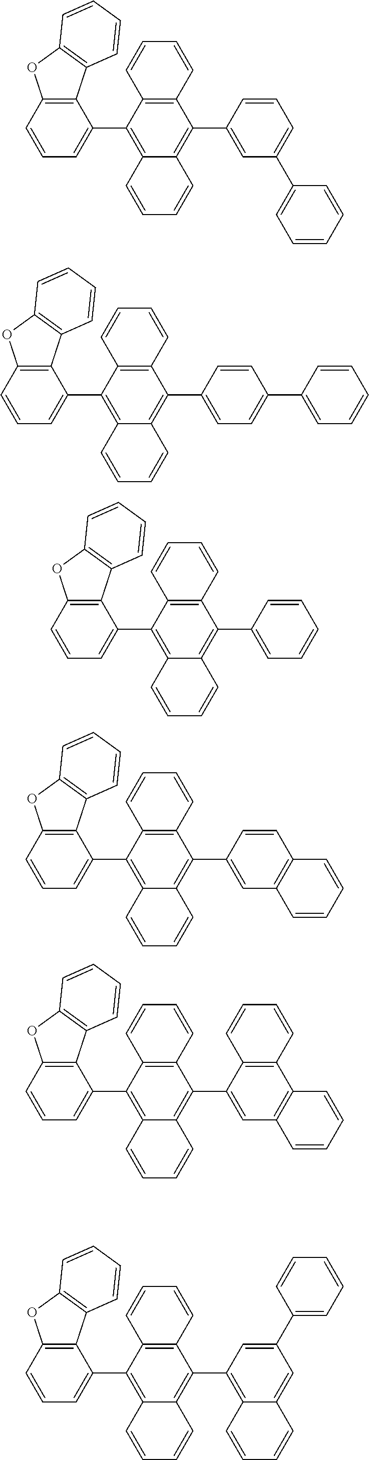

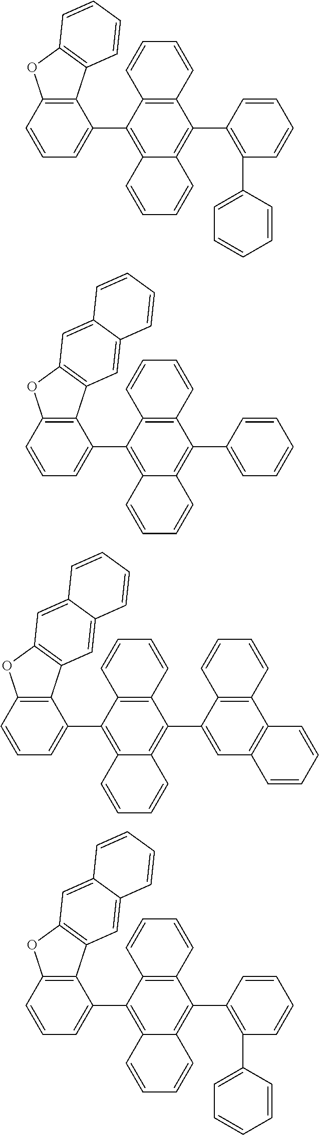

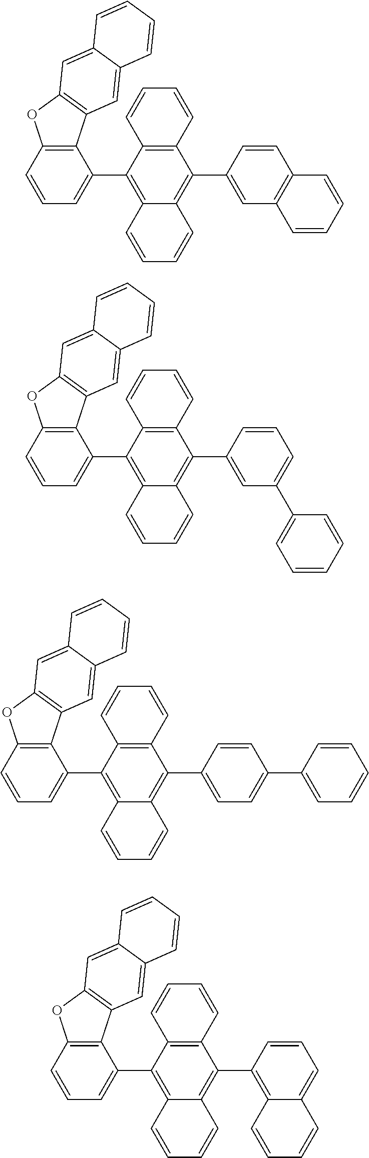

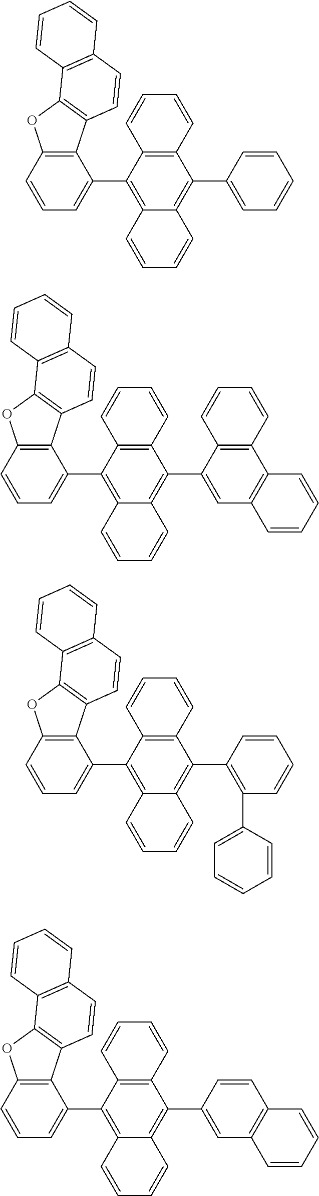 [SMILES: c1ccc(-c2c3ccccc3c(-c3cccc4oc5c6ccccc6ccc5c34)c3ccccc23)cc1 ; c1ccc2c(c1)cc(-c1c3ccccc3c(-c3cccc4oc5c6ccccc6ccc5c34)c3ccccc13)c1ccccc12 ; c1ccc(-c2ccccc2-c2c3ccccc3c(-c3cccc4oc5c6ccccc6ccc5c34)c3ccccc23)cc1 ; c1ccc2cc(-c3c4ccccc4c(-c4cccc5oc6c7ccccc7ccc6c45)c4ccccc34)ccc2c1]

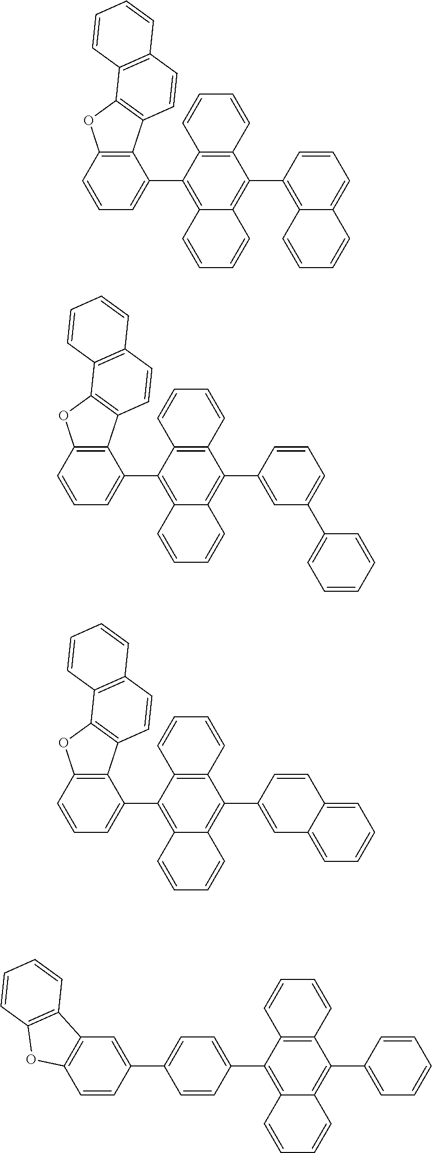 [SMILES: c1ccc2c(-c3c4ccccc4c(-c4cccc5oc6c7ccccc7ccc6c45)c4ccccc34)cccc2c1 ; c1ccc(-c2cccc(-c3c4ccccc4c(-c4cccc5oc6c7ccccc7ccc6c45)c4ccccc34)c2)cc1 ; c1ccc2cc(-c3c4ccccc4c(-c4cccc5oc6c7ccccc7ccc6c45)c4ccccc34)ccc2c1 ; c1ccc(-c2c3ccccc3c(-c3ccc(-c4ccc5oc6ccccc6c5c4)cc3)c3ccccc23)cc1]

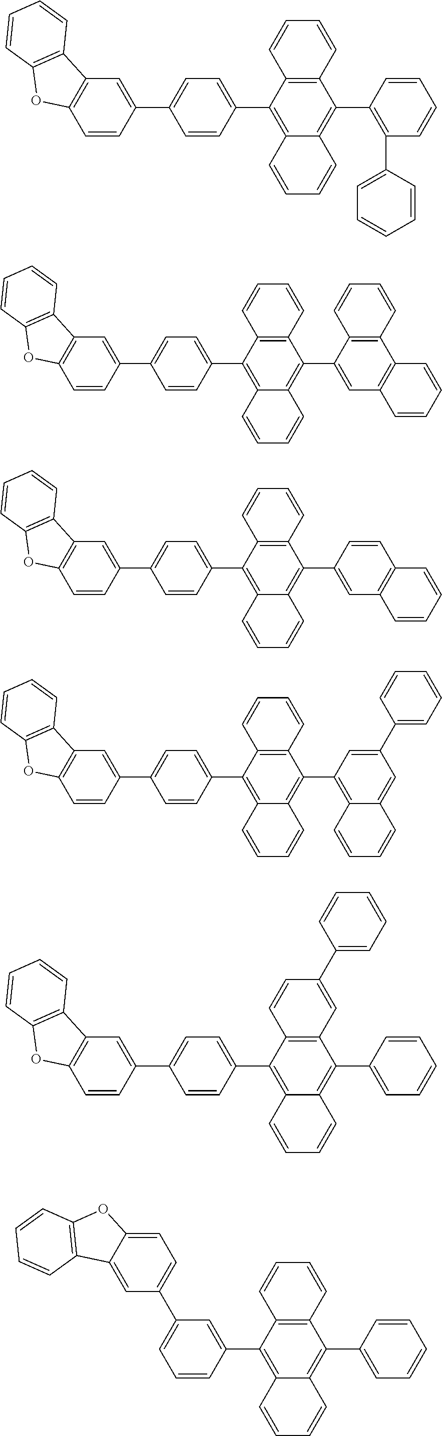

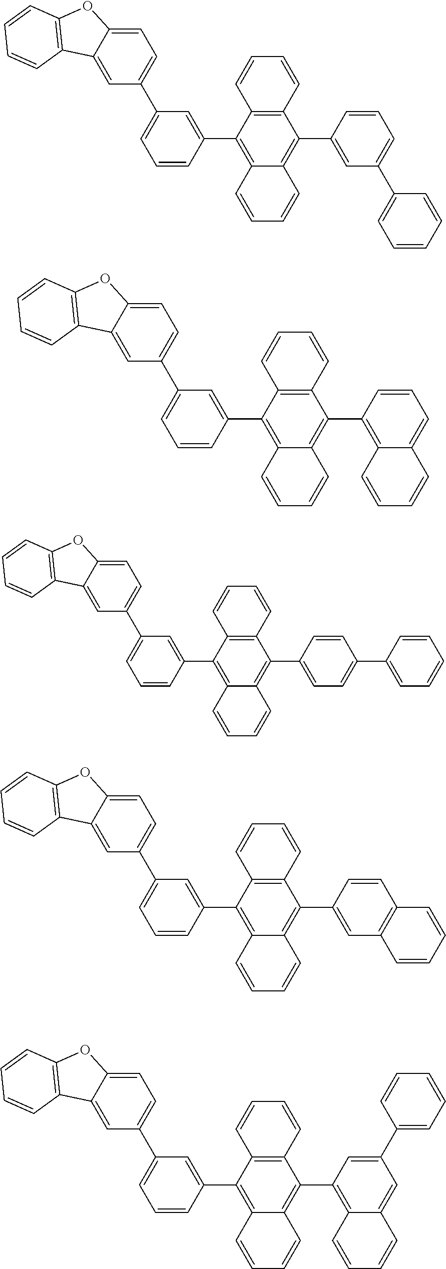 [SMILES: c1ccc(-c2cccc(-c3c4ccccc4c(-c4cccc(-c5ccc6oc7ccccc7c6c5)c4)c4ccccc34)c2)cc1 ; c1cc(-c2ccc3oc4ccccc4c3c2)cc(-c2c3ccccc3c(-c3cccc4ccccc34)c3ccccc23)c1 ; c1ccc(-c2ccc(-c3c4ccccc4c(-c4cccc(-c5ccc6oc7ccccc7c6c5)c4)c4ccccc34)cc2)cc1 ; c1cc(-c2ccc3oc4ccccc4c3c2)cc(-c2c3ccccc3c(-c3ccc4ccccc4c3)c3ccccc23)c1 ; c1ccc(-c2cc(-c3c4ccccc4c(-c4cccc(-c5ccc6oc7ccccc7c6c5)c4)c4ccccc34)c3ccccc3c2)cc1]

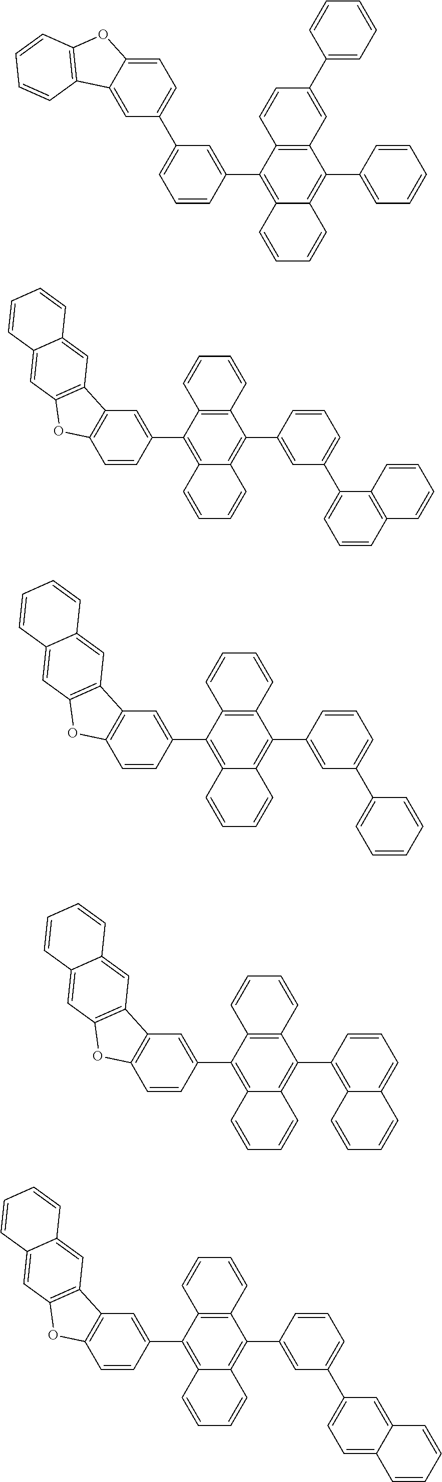 [SMILES: c1ccc(-c2ccc3c(-c4cccc(-c5ccc6oc7ccccc7c6c5)c4)c4ccccc4c(-c4ccccc4)c3c2)cc1 ; c1cc(-c2cccc3ccccc23)cc(-c2c3ccccc3c(-c3ccc4oc5cc6ccccc6cc5c4c3)c3ccccc23)c1 ; c1ccc(-c2cccc(-c3c4ccccc4c(-c4ccc5oc6cc7ccccc7cc6c5c4)c4ccccc34)c2)cc1 ; c1ccc2cc3c(cc2c1)oc1ccc(-c2c4ccccc4c(-c4cccc5ccccc45)c4ccccc24)cc13 ; c1cc(-c2ccc3ccccc3c2)cc(-c2c3ccccc3c(-c3ccc4oc5cc6ccccc6cc5c4c3)c3ccccc23)c1]

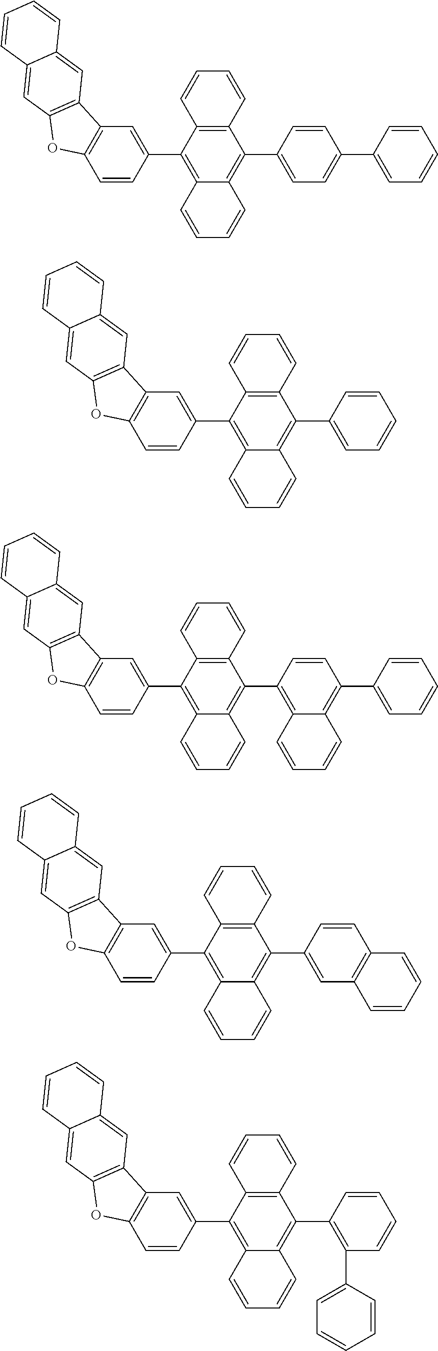 [SMILES: c1ccc(-c2ccc(-c3c4ccccc4c(-c4ccc5oc6cc7ccccc7cc6c5c4)c4ccccc34)cc2)cc1 ; c1ccc(-c2c3ccccc3c(-c3ccc4oc5cc6ccccc6cc5c4c3)c3ccccc23)cc1 ; c1ccc(-c2ccc(-c3c4ccccc4c(-c4ccc5oc6cc7ccccc7cc6c5c4)c4ccccc34)c3ccccc23)cc1 ; c1ccc2cc(-c3c4ccccc4c(-c4ccc5oc6cc7ccccc7cc6c5c4)c4ccccc34)ccc2c1 ; c1ccc(-c2ccccc2-c2c3ccccc3c(-c3ccc4oc5cc6ccccc6cc5c4c3)c3ccccc23)cc1]

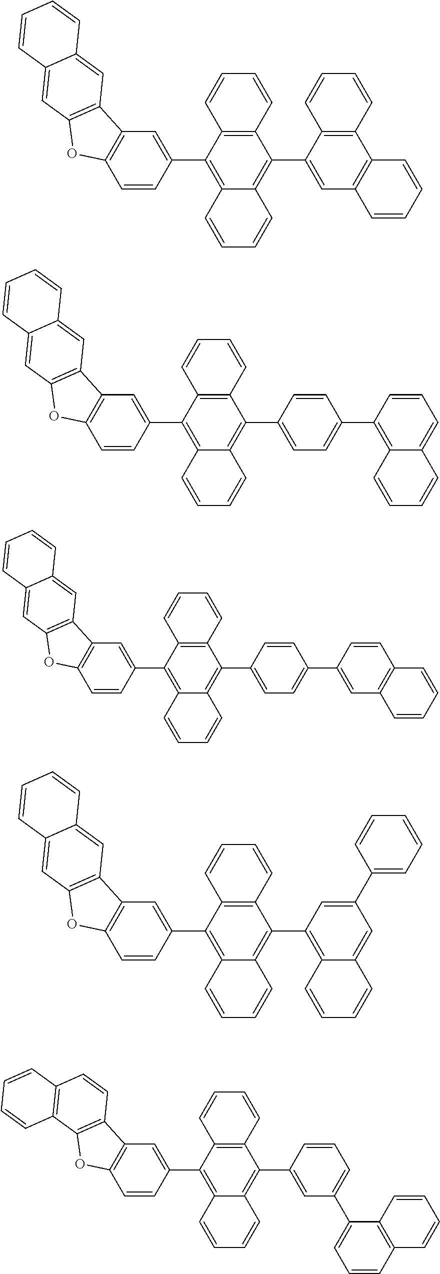 [SMILES: c1ccc2cc3c(cc2c1)oc1ccc(-c2c4ccccc4c(-c4cc5ccccc5c5ccccc45)c4ccccc24)cc13 ; c1ccc2cc3c(cc2c1)oc1ccc(-c2c4ccccc4c(-c4ccc(-c5cccc6ccccc56)cc4)c4ccccc24)cc13 ; c1ccc2cc(-c3ccc(-c4c5ccccc5c(-c5ccc6oc7cc8ccccc8cc7c6c5)c5ccccc45)cc3)ccc2c1 ; c1ccc(-c2cc(-c3c4ccccc4c(-c4ccc5oc6cc7ccccc7cc6c5c4)c4ccccc34)c3ccccc3c2)cc1 ; c1cc(-c2cccc3ccccc23)cc(-c2c3ccccc3c(-c3ccc4oc5c6ccccc6ccc5c4c3)c3ccccc23)c1]

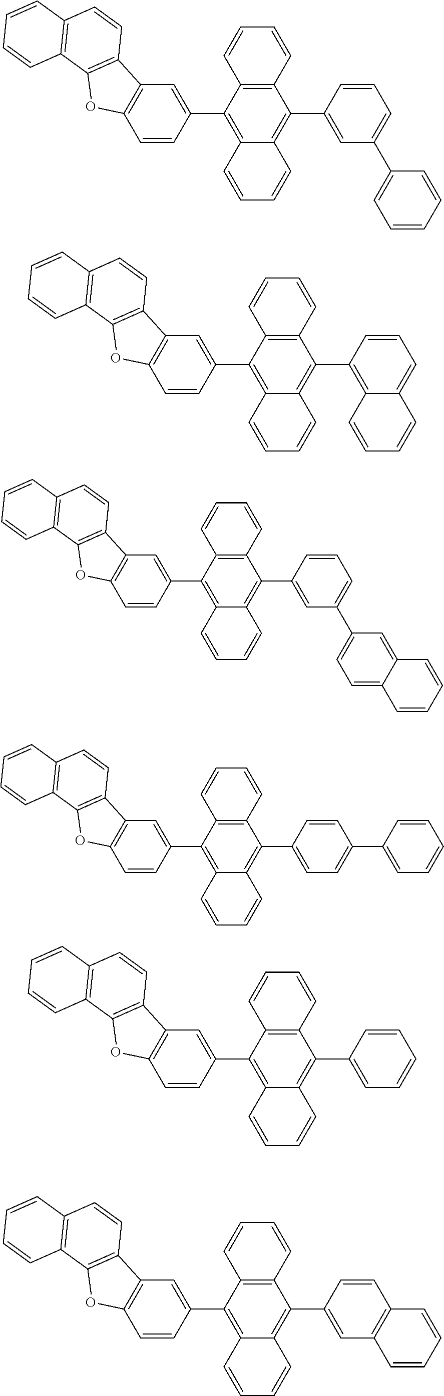 [SMILES: c1ccc(-c2cccc(-c3c4ccccc4c(-c4ccc5oc6c7ccccc7ccc6c5c4)c4ccccc34)c2)cc1 ; c1ccc2c(-c3c4ccccc4c(-c4ccc5oc6c7ccccc7ccc6c5c4)c4ccccc34)cccc2c1 ; c1cc(-c2ccc3ccccc3c2)cc(-c2c3ccccc3c(-c3ccc4oc5c6ccccc6ccc5c4c3)c3ccccc23)c1 ; c1ccc(-c2ccc(-c3c4ccccc4c(-c4ccc5oc6c7ccccc7ccc6c5c4)c4ccccc34)cc2)cc1 ; c1ccc(-c2c3ccccc3c(-c3ccc4oc5c6ccccc6ccc5c4c3)c3ccccc23)cc1 ; c1ccc2cc(-c3c4ccccc4c(-c4ccc5oc6c7ccccc7ccc6c5c4)c4ccccc34)ccc2c1]

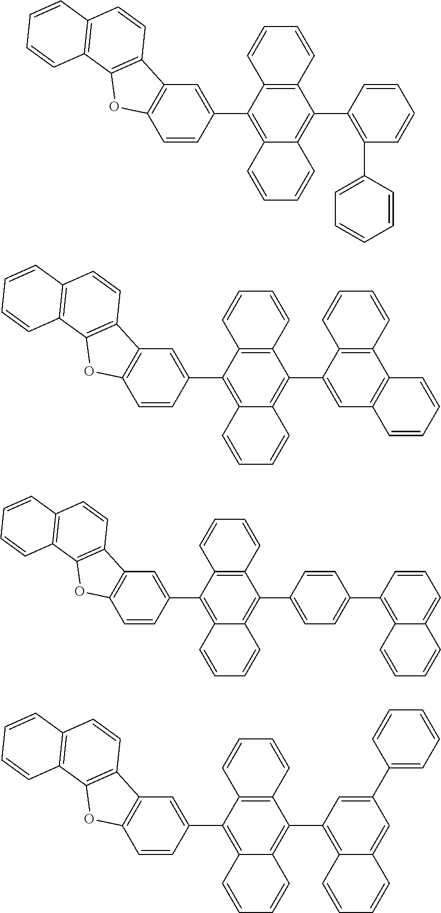

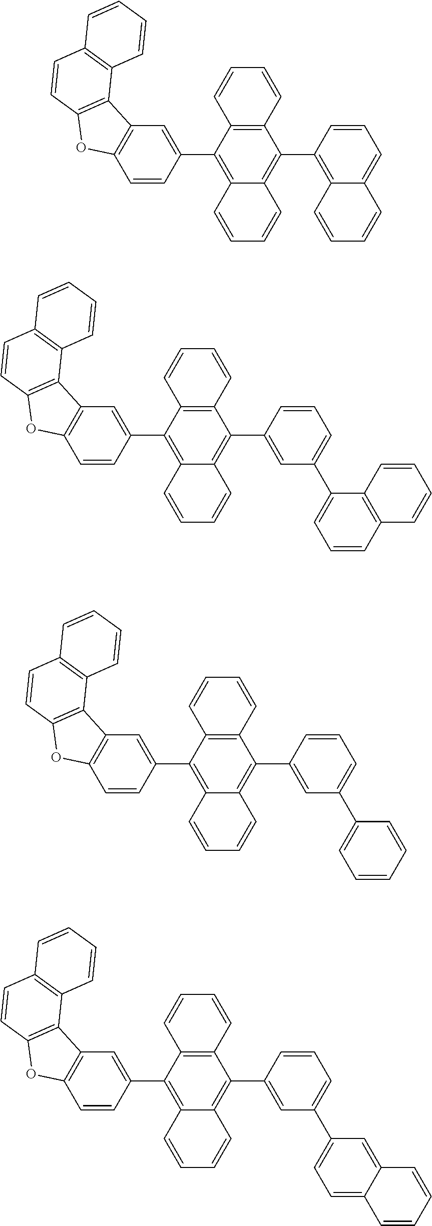 [SMILES: c1ccc2c(-c3c4ccccc4c(-c4ccc5oc6ccc7ccccc7c6c5c4)c4ccccc34)cccc2c1 ; c1cc(-c2cccc3ccccc23)cc(-c2c3ccccc3c(-c3ccc4oc5ccc6ccccc6c5c4c3)c3ccccc23)c1 ; c1ccc(-c2cccc(-c3c4ccccc4c(-c4ccc5oc6ccc7ccccc7c6c5c4)c4ccccc34)c2)cc1 ; c1cc(-c2ccc3ccccc3c2)cc(-c2c3ccccc3c(-c3ccc4oc5ccc6ccccc6c5c4c3)c3ccccc23)c1]

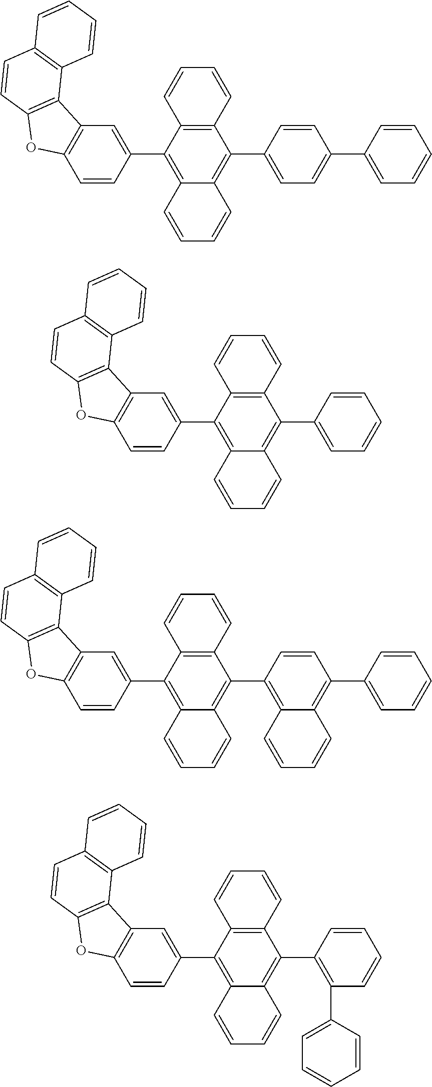 [SMILES: c1ccc(-c2ccc(-c3c4ccccc4c(-c4ccc5oc6ccc7ccccc7c6c5c4)c4ccccc34)cc2)cc1 ; c1ccc(-c2c3ccccc3c(-c3ccc4oc5ccc6ccccc6c5c4c3)c3ccccc23)cc1 ; c1ccc(-c2ccc(-c3c4ccccc4c(-c4ccc5oc6ccc7ccccc7c6c5c4)c4ccccc34)c3ccccc23)cc1 ; c1ccc(-c2ccccc2-c2c3ccccc3c(-c3ccc4oc5ccc6ccccc6c5c4c3)c3ccccc23)cc1]

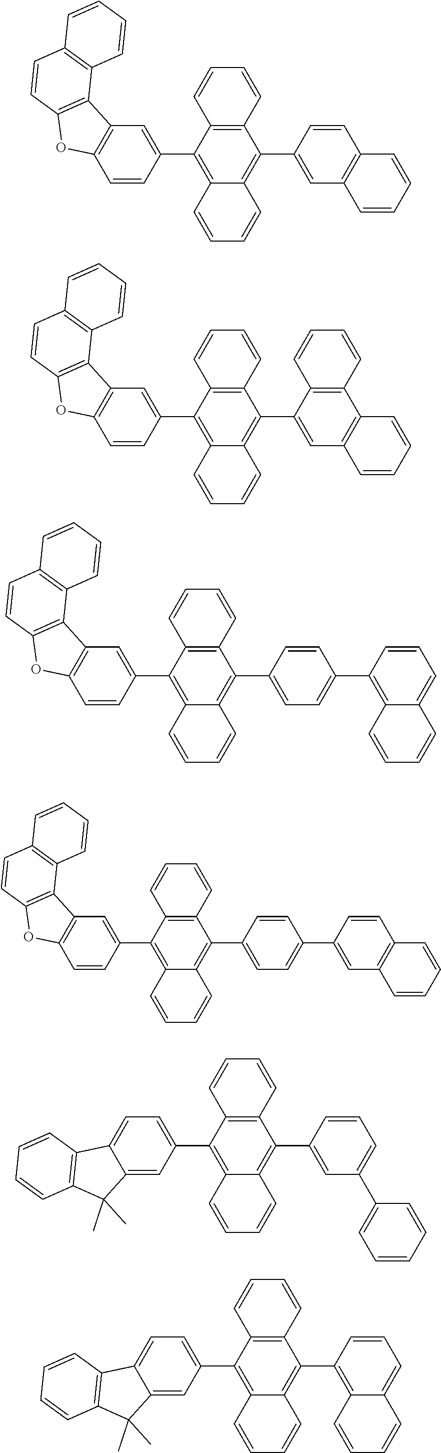

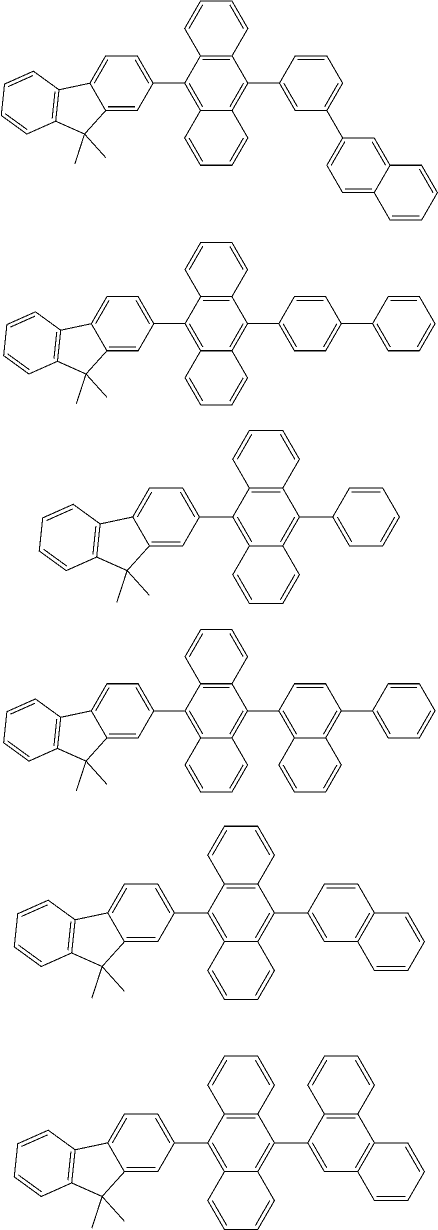

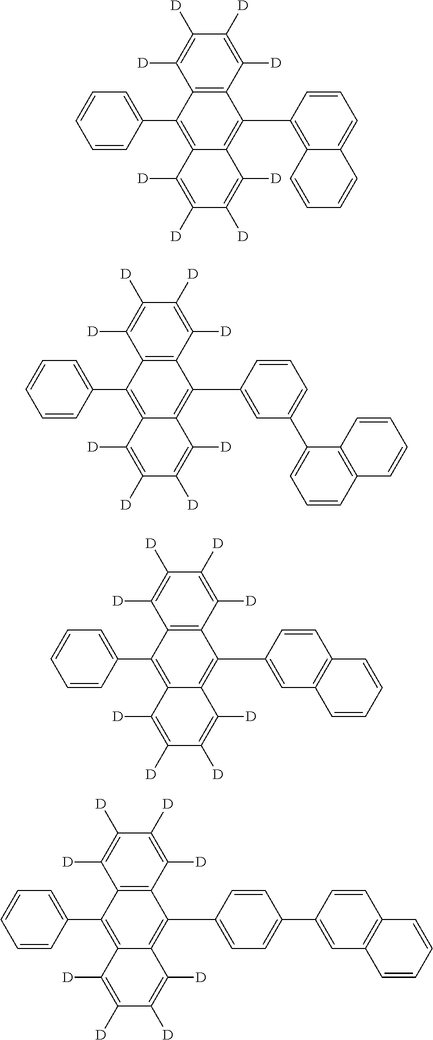

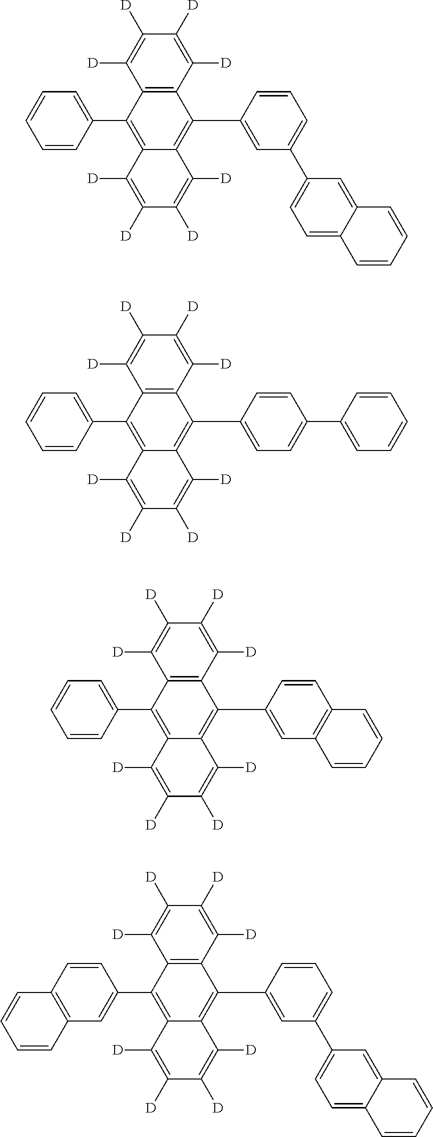 [SMILES: [2H]c1c([2H])c([2H])c2c(-c3cccc(-c4ccc5ccccc5c4)c3)c3c([2H])c([2H])c([2H])c([2H])c3c(-c3ccccc3)c2c1[2H] ; [2H]c1c([2H])c([2H])c2c(-c3ccc(-c4ccccc4)cc3)c3c([2H])c([2H])c([2H])c([2H])c3c(-c3ccccc3)c2c1[2H] ; [2H]c1c([2H])c([2H])c2c(-c3ccc4ccccc4c3)c3c([2H])c([2H])c([2H])c([2H])c3c(-c3ccccc3)c2c1[2H] ; [2H]c1c([2H])c([2H])c2c(-c3ccc4ccccc4c3)c3c([2H])c([2H])c([2H])c([2H])c3c(-c3cccc(-c4ccc5ccccc5c4)c3)c2c1[2H]]

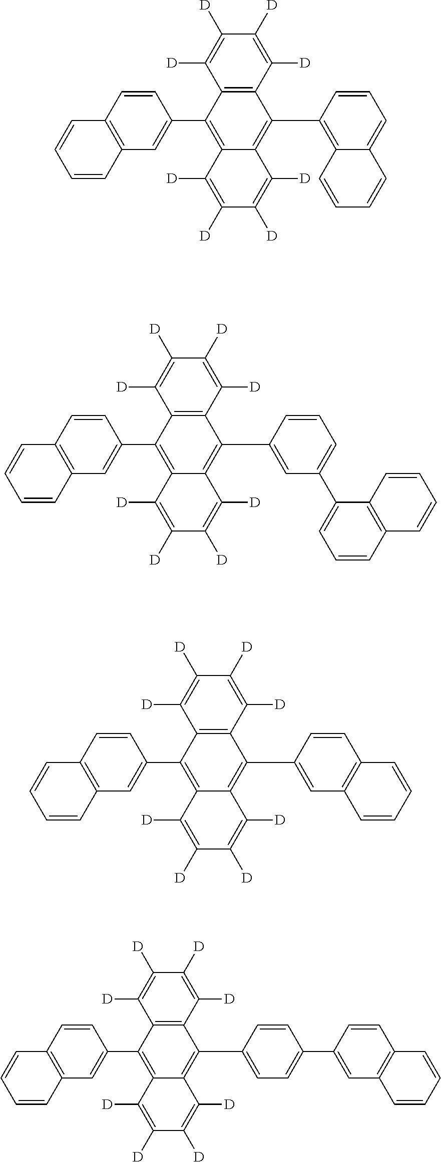 [SMILES: [2H]c1c([2H])c([2H])c2c(-c3cccc4ccccc34)c3c([2H])c([2H])c([2H])c([2H])c3c(-c3ccc4ccccc4c3)c2c1[2H] ; [2H]c1c([2H])c([2H])c2c(-c3ccc4ccccc4c3)c3c([2H])c([2H])c([2H])c([2H])c3c(-c3cccc(-c4cccc5ccccc45)c3)c2c1[2H] ; [2H]c1c([2H])c([2H])c2c(-c3ccc4ccccc4c3)c3c([2H])c([2H])c([2H])c([2H])c3c(-c3ccc4ccccc4c3)c2c1[2H] ; [2H]c1c([2H])c([2H])c2c(-c3ccc4ccccc4c3)c3c([2H])c([2H])c([2H])c([2H])c3c(-c3ccc(-c4ccc5ccccc5c4)cc3)c2c1[2H]]

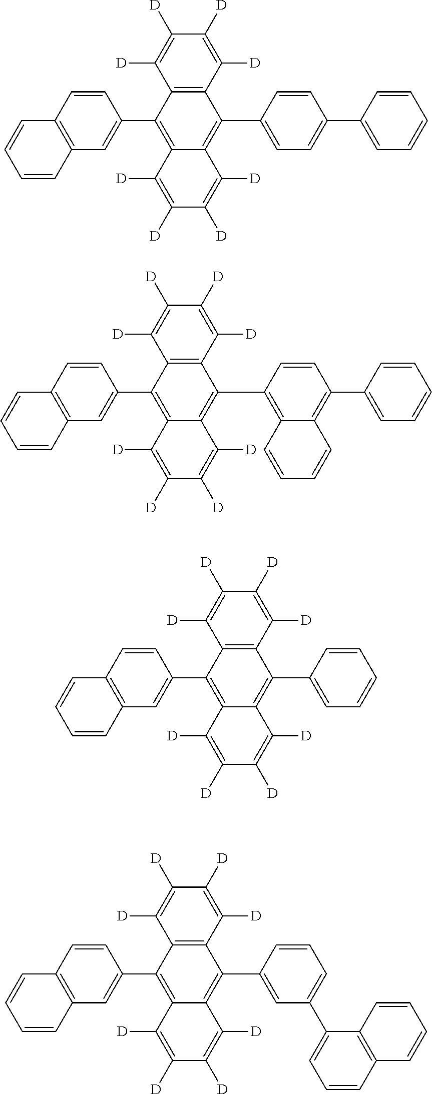

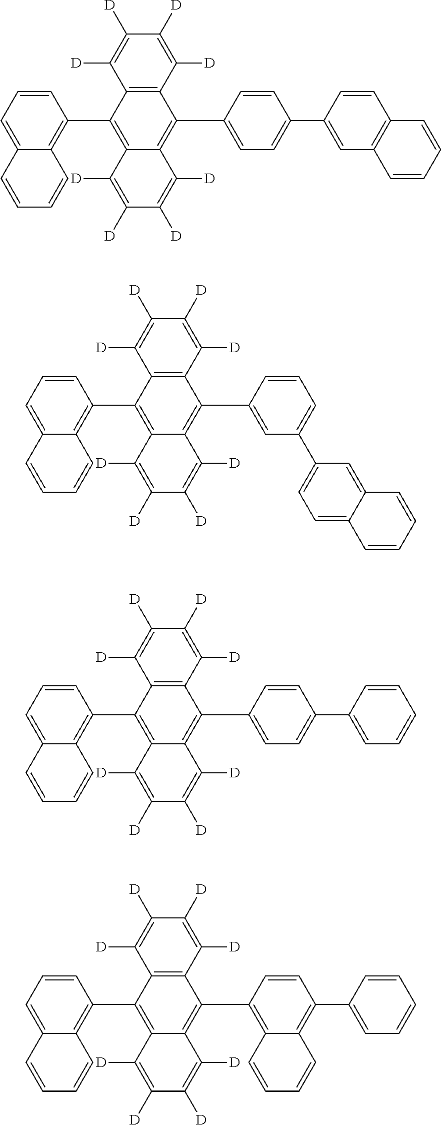 [SMILES: [2H]c1c([2H])c([2H])c2c(-c3cccc4ccccc34)c3c([2H])c([2H])c([2H])c([2H])c3c(-c3ccc(-c4ccc5ccccc5c4)cc3)c2c1[2H] ; [2H]c1c([2H])c([2H])c2c(-c3cccc4ccccc34)c3c([2H])c([2H])c([2H])c([2H])c3c(-c3cccc(-c4ccc5ccccc5c4)c3)c2c1[2H] ; [2H]c1c([2H])c([2H])c2c(-c3cccc4ccccc34)c3c([2H])c([2H])c([2H])c([2H])c3c(-c3ccc(-c4ccccc4)cc3)c2c1[2H] ; [2H]c1c([2H])c([2H])c2c(-c3ccc(-c4ccccc4)c4ccccc34)c3c([2H])c([2H])c([2H])c([2H])c3c(-c3cccc4ccccc34)c2c1[2H]]

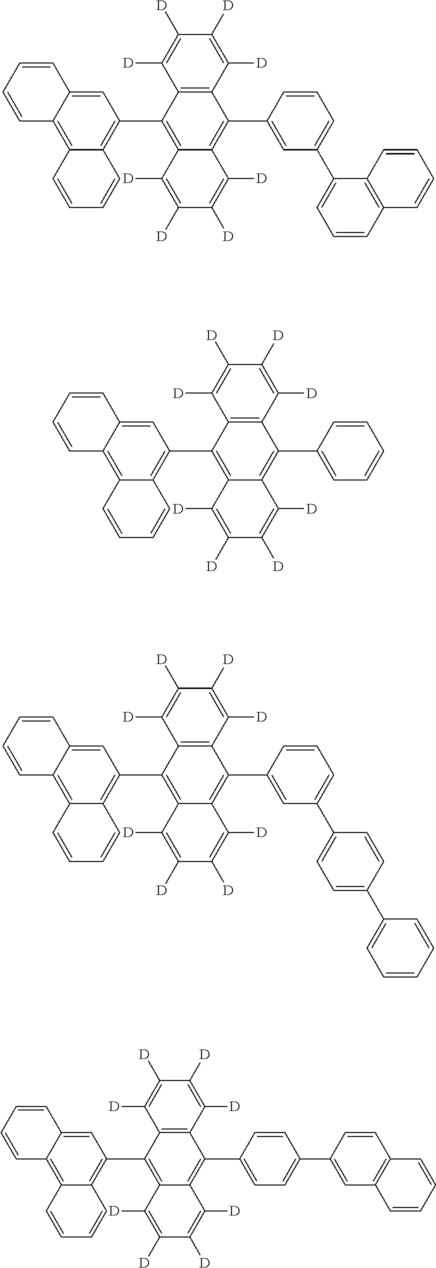 [SMILES: [2H]c1c([2H])c([2H])c2c(-c3cc4ccccc4c4ccccc34)c3c([2H])c([2H])c([2H])c([2H])c3c(-c3cccc(-c4cccc5ccccc45)c3)c2c1[2H] ; [2H]c1c([2H])c([2H])c2c(-c3cc4ccccc4c4ccccc34)c3c([2H])c([2H])c([2H])c([2H])c3c(-c3ccccc3)c2c1[2H] ; [2H]c1c([2H])c([2H])c2c(-c3cc4ccccc4c4ccccc34)c3c([2H])c([2H])c([2H])c([2H])c3c(-c3cccc(-c4ccc(-c5ccccc5)cc4)c3)c2c1[2H] ; [2H]c1c([2H])c([2H])c2c(-c3cc4ccccc4c4ccccc34)c3c([2H])c([2H])c([2H])c([2H])c3c(-c3ccc(-c4ccc5ccccc5c4)cc3)c2c1[2H]]

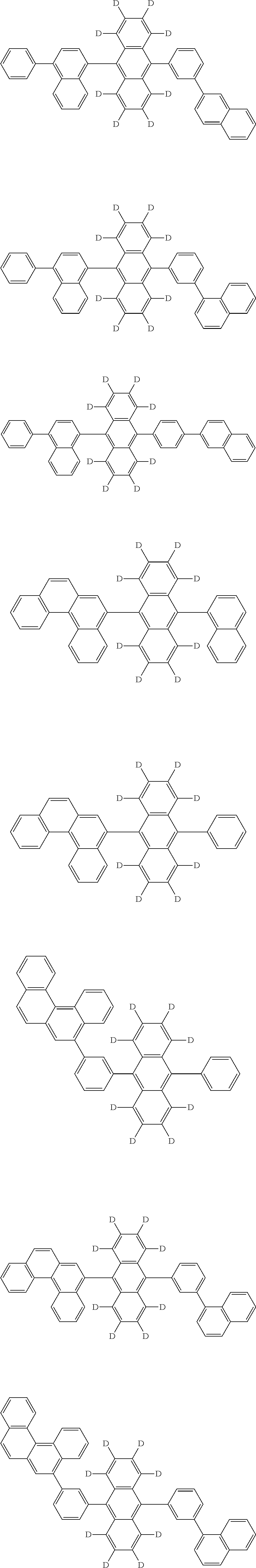

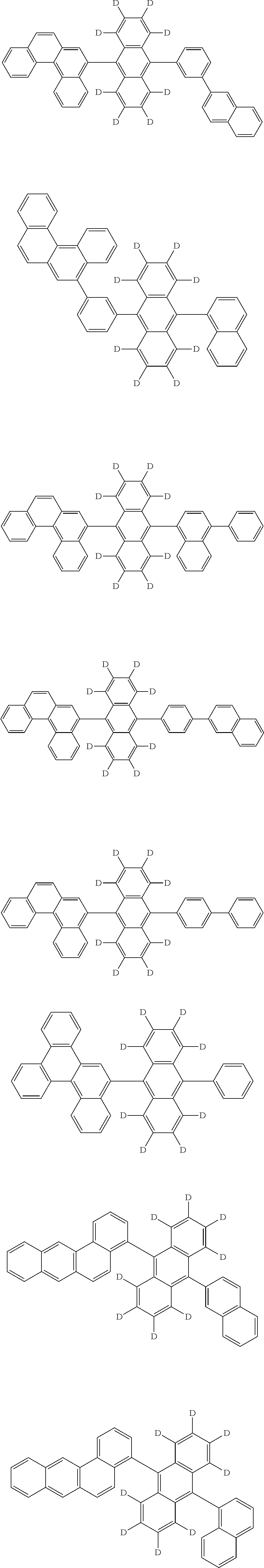

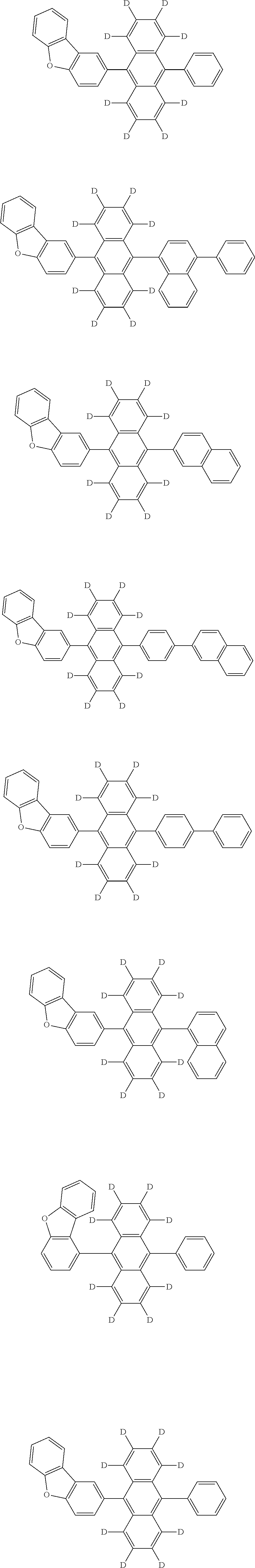

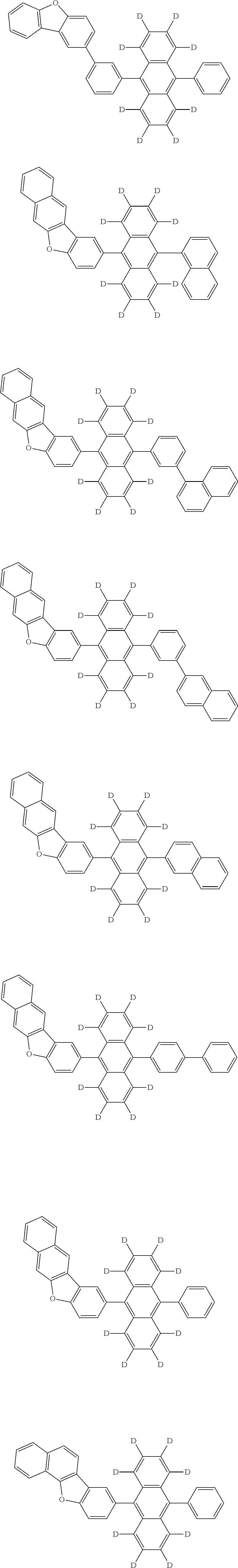

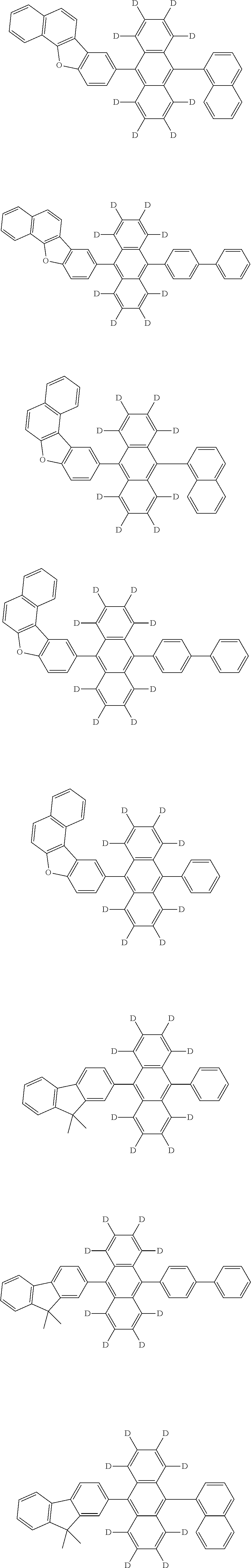

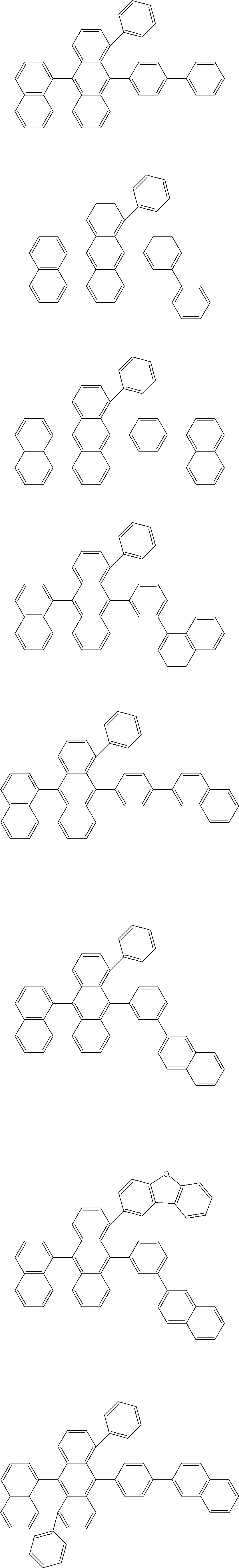

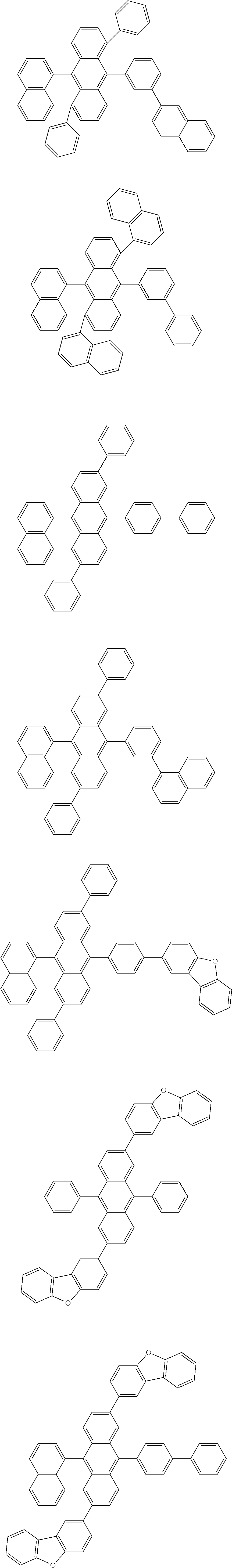

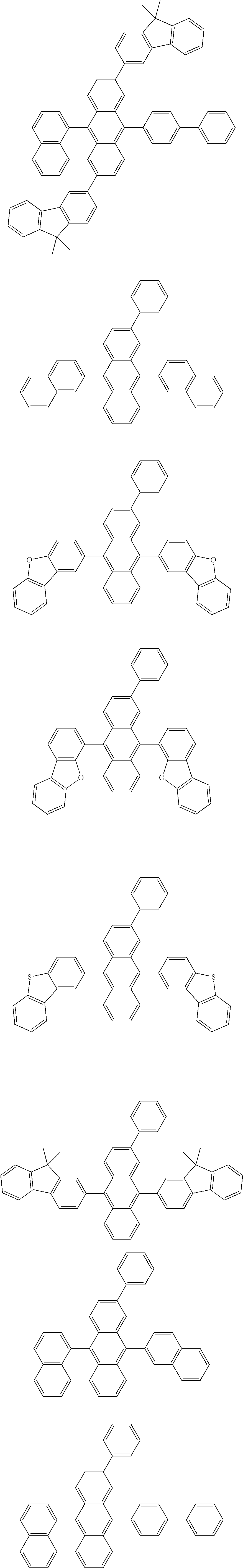

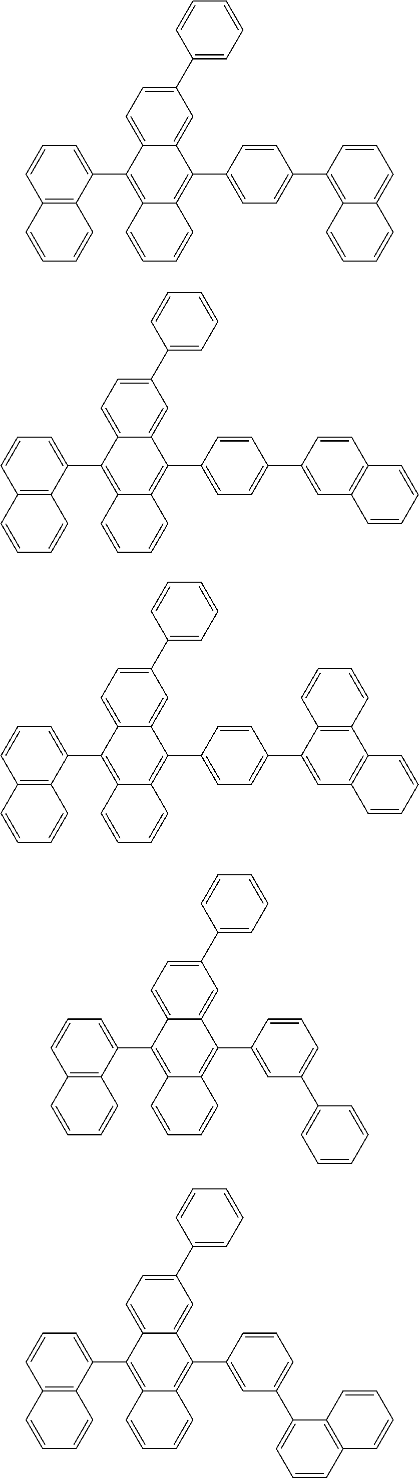

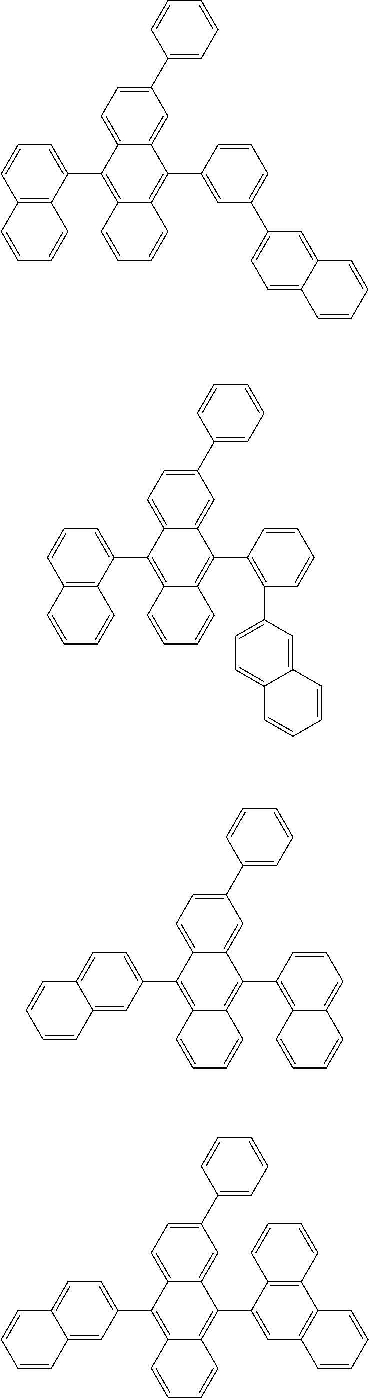

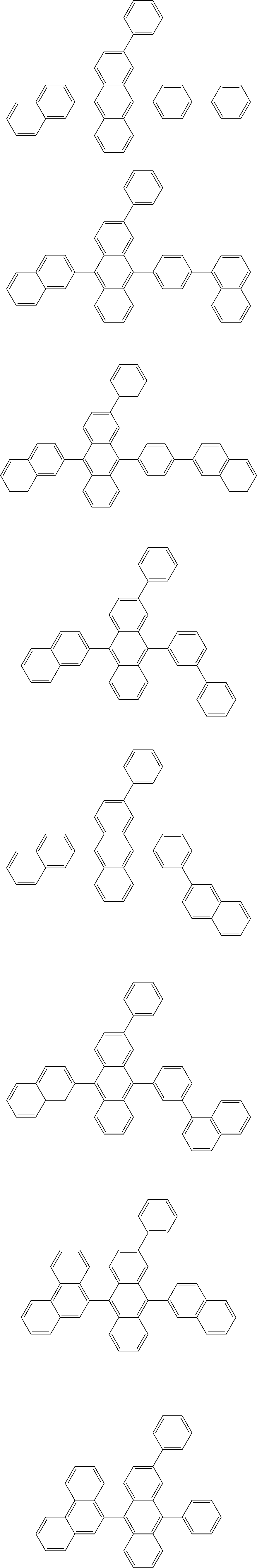

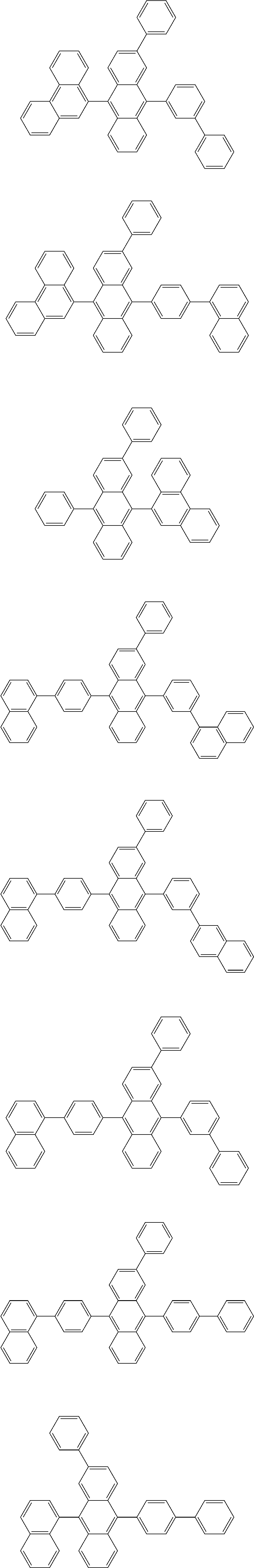

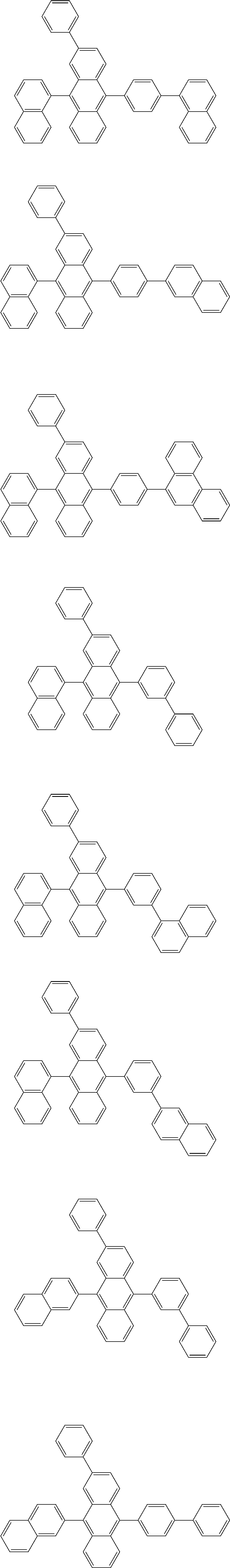

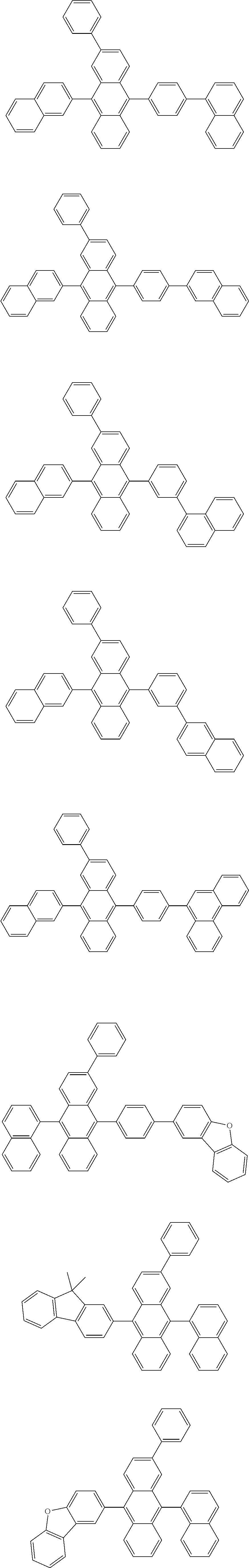

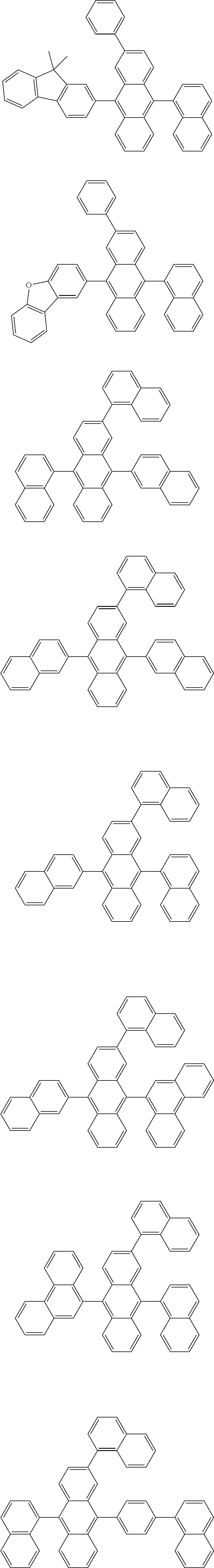

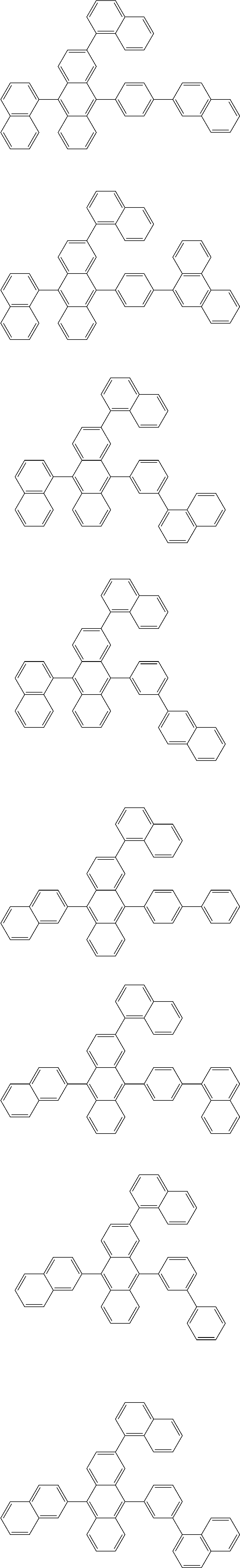

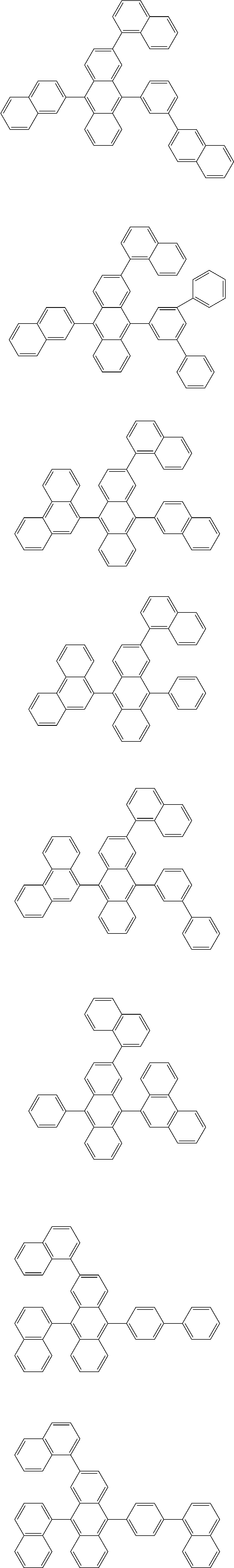

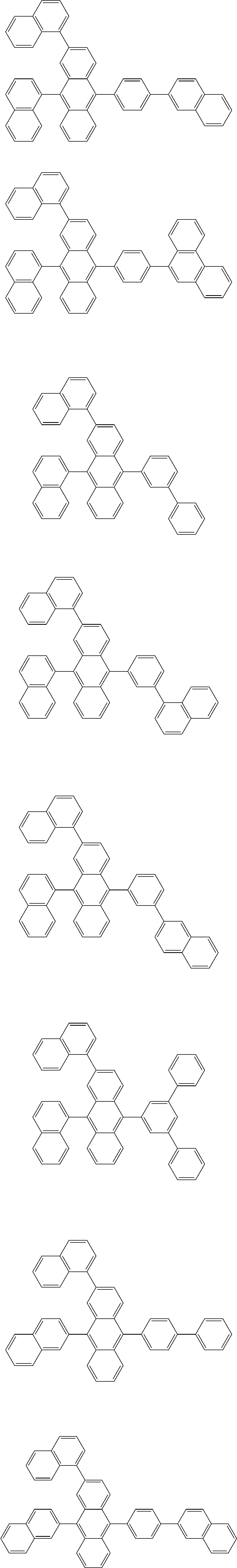

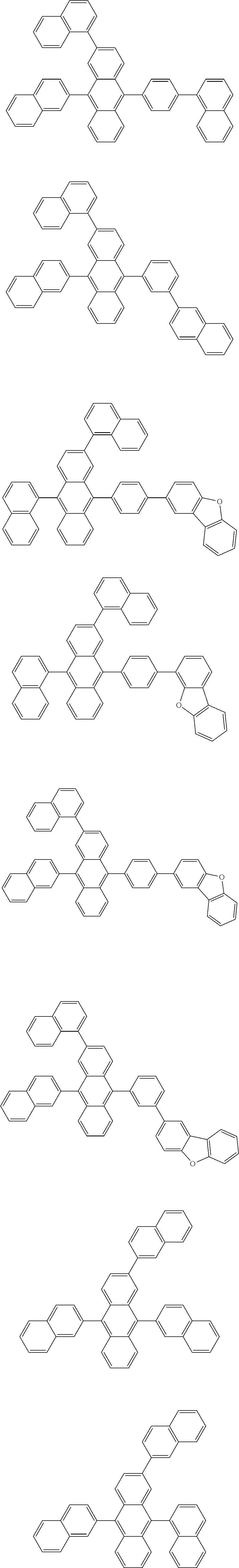

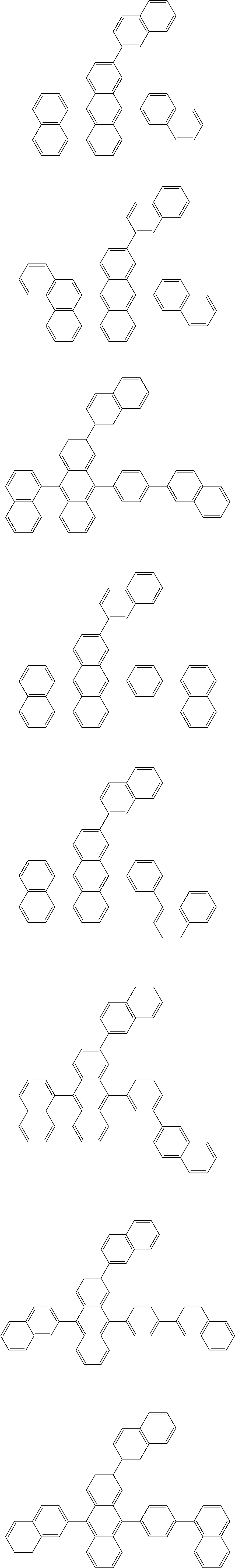

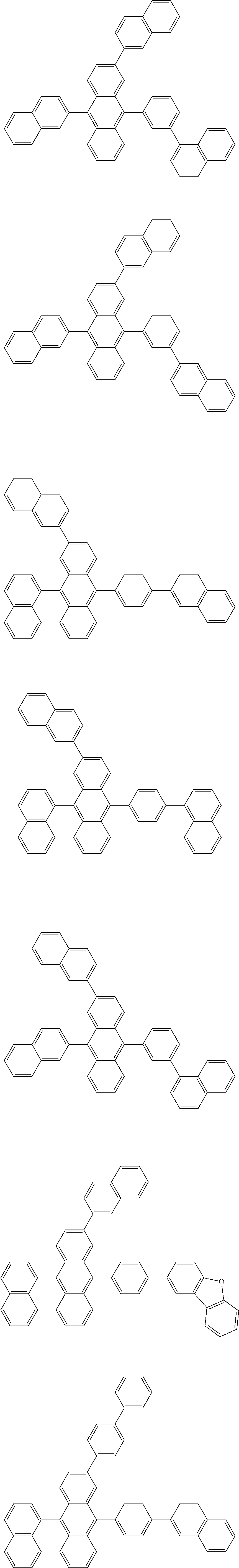

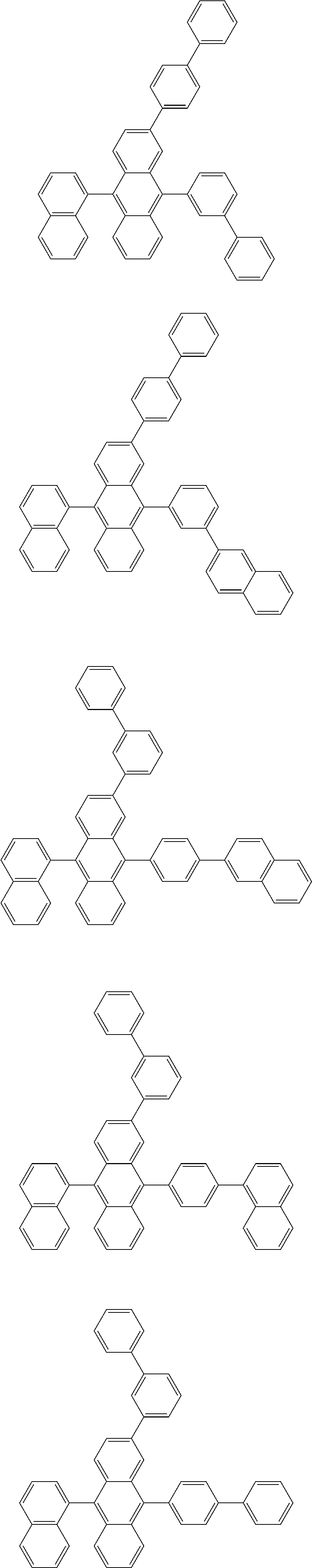

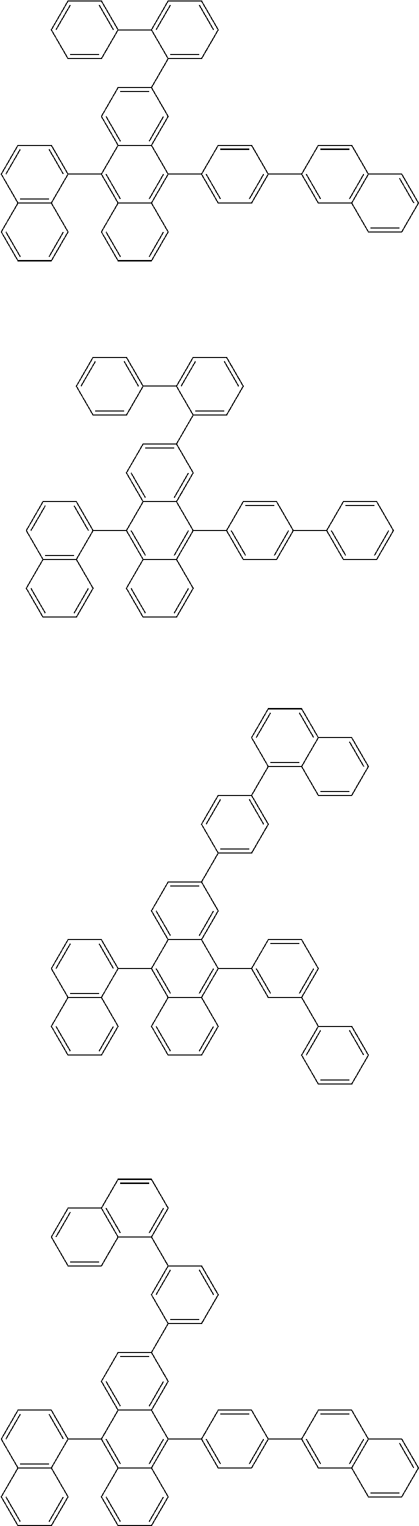

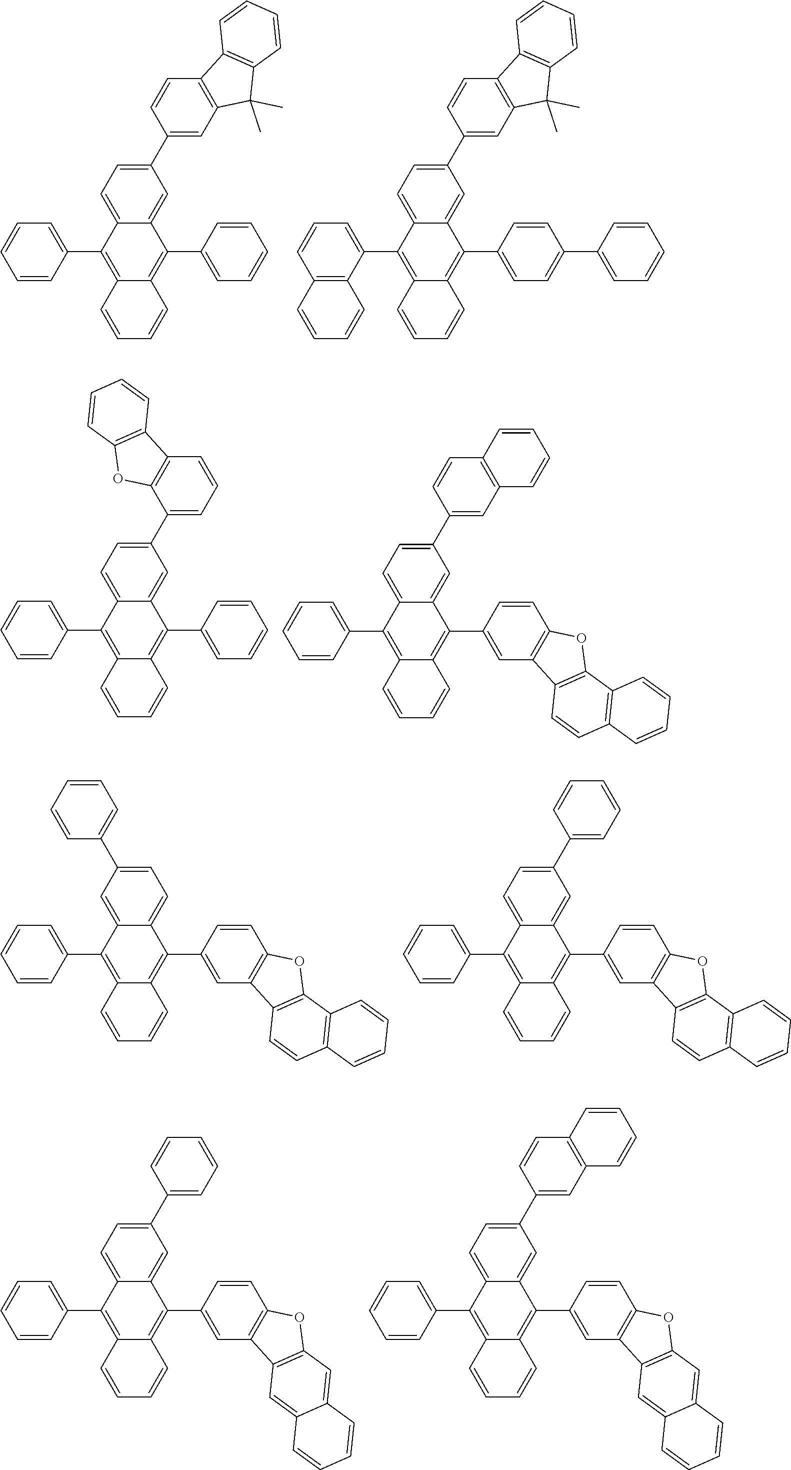

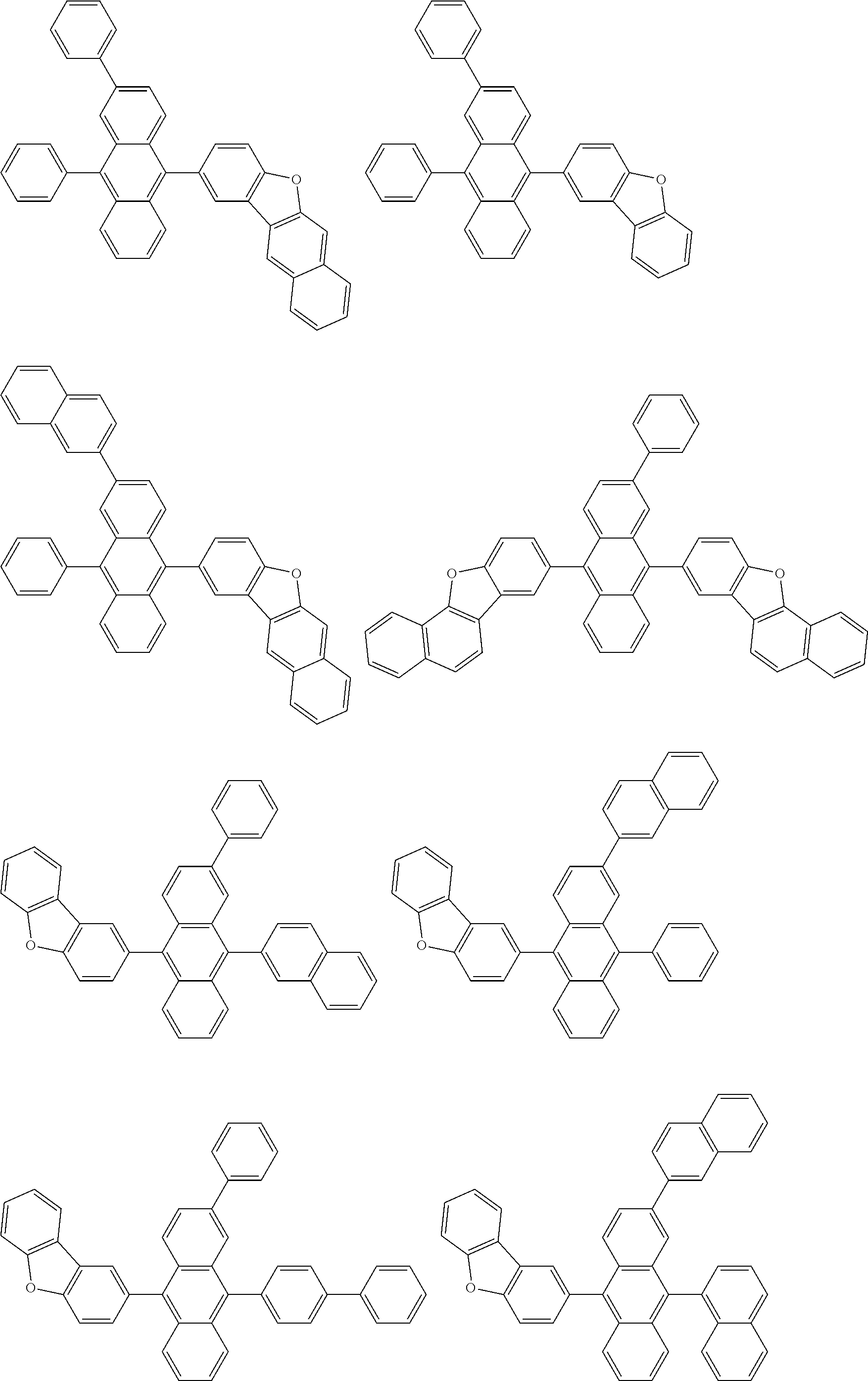

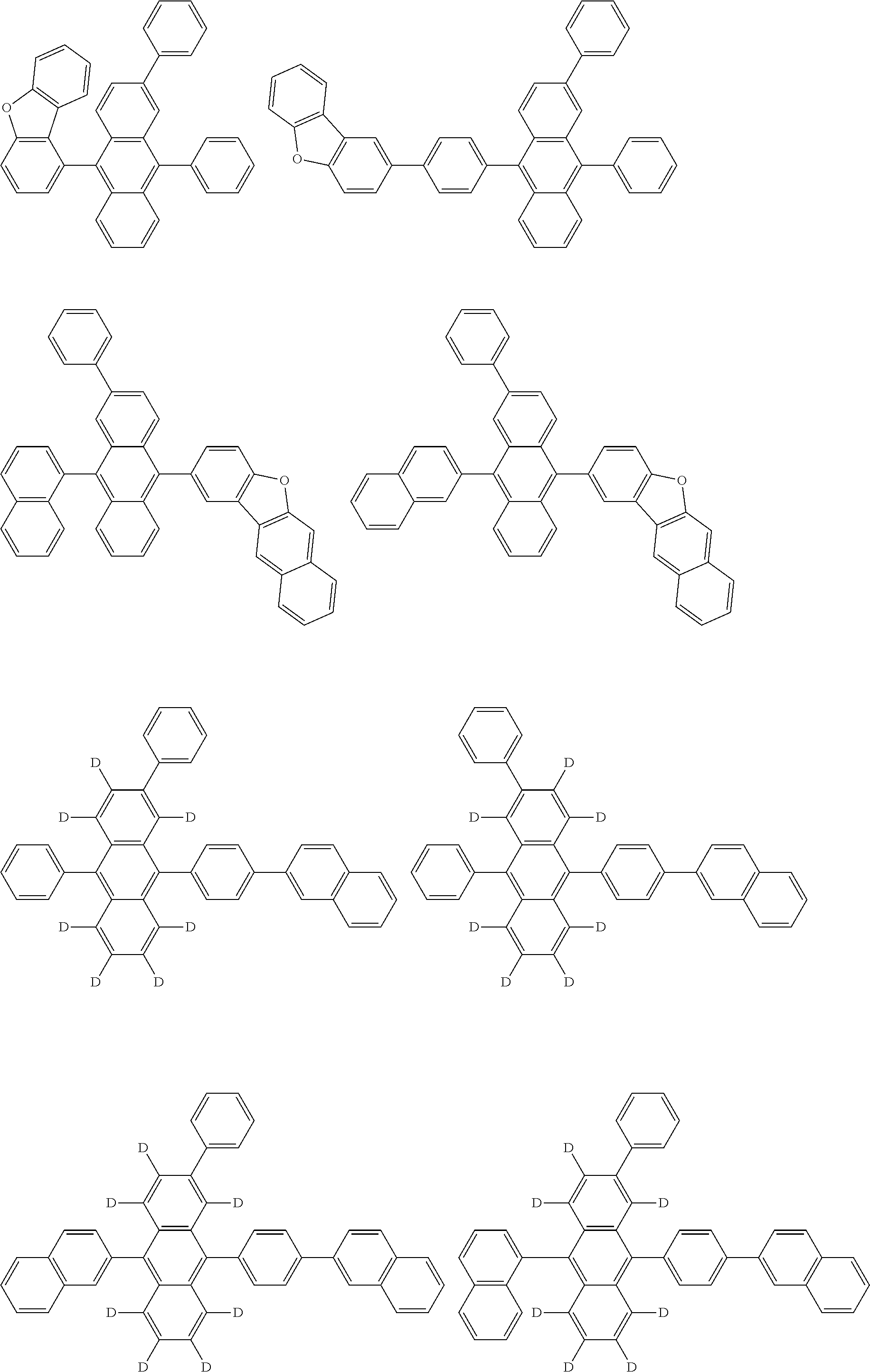

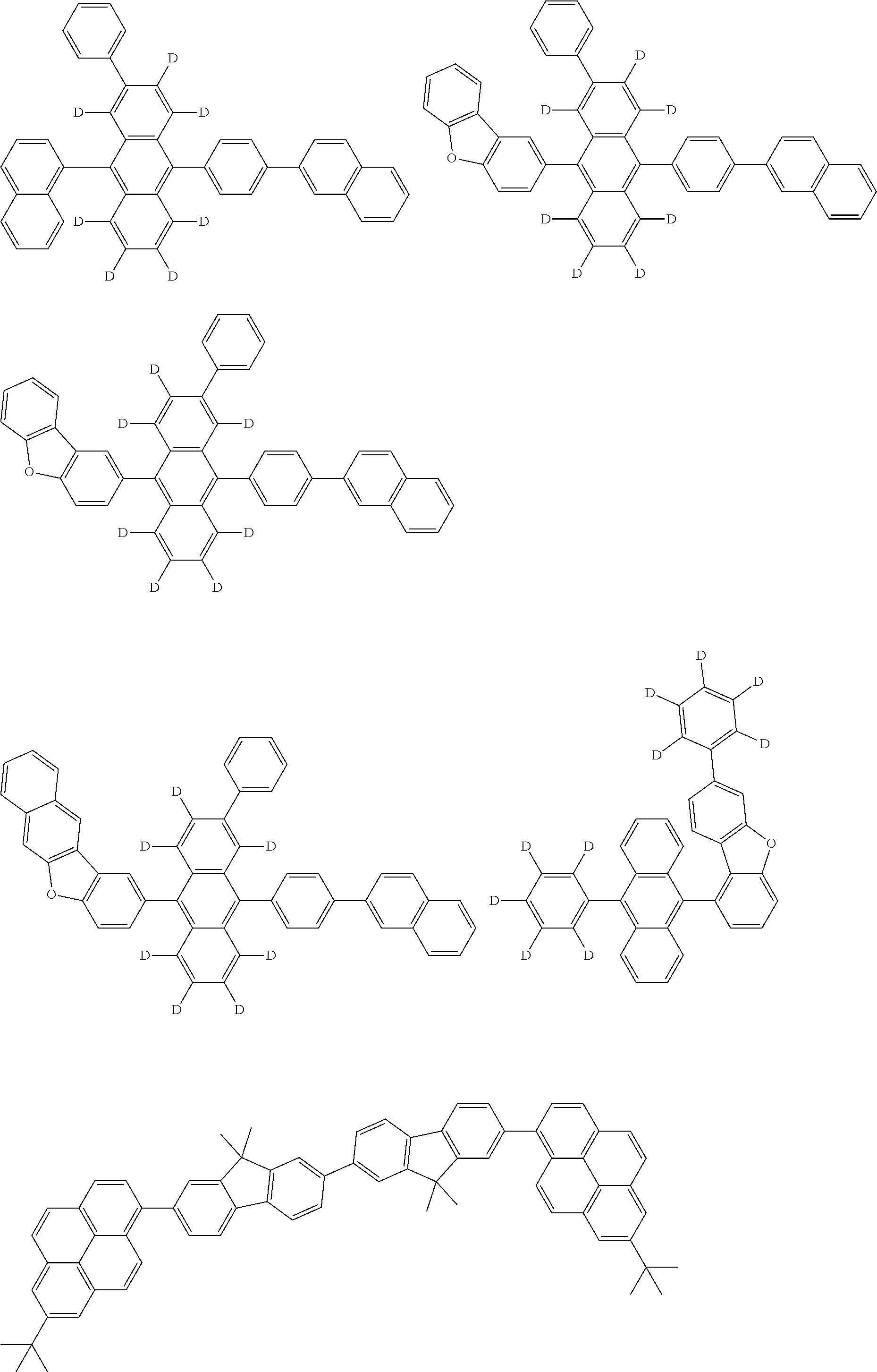

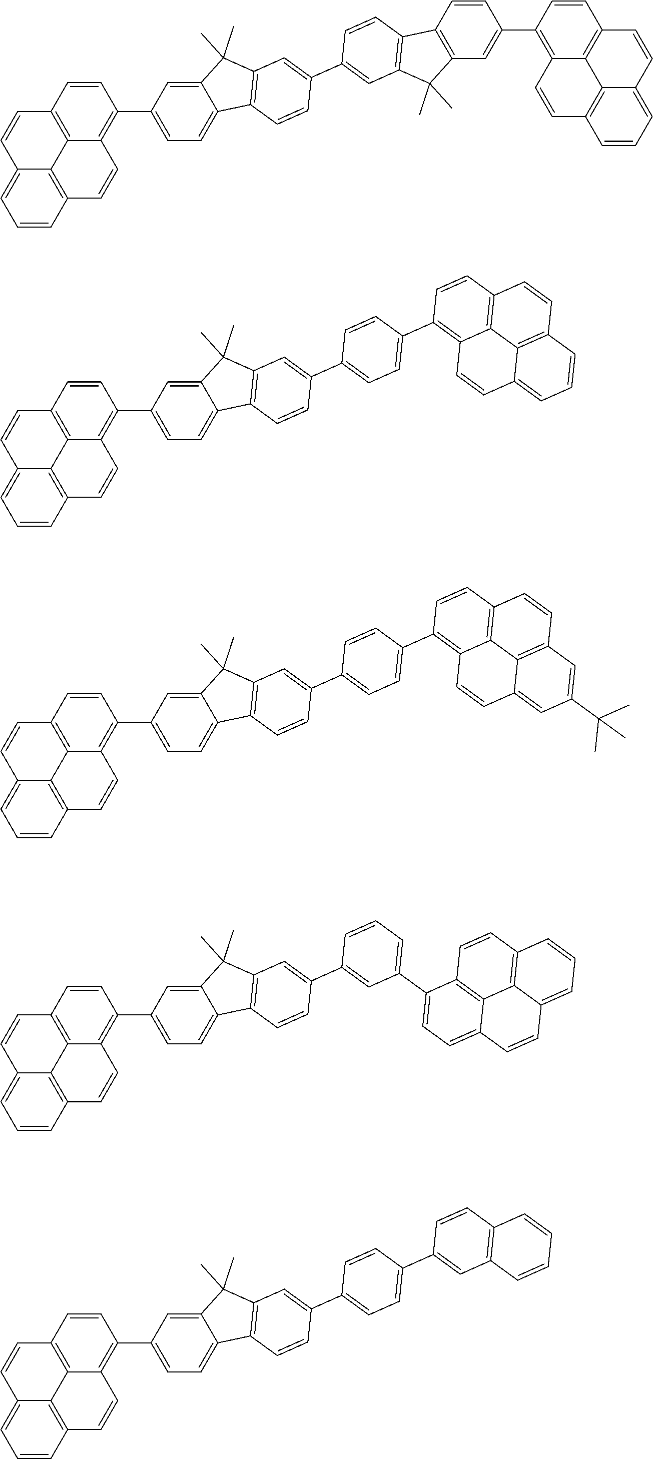

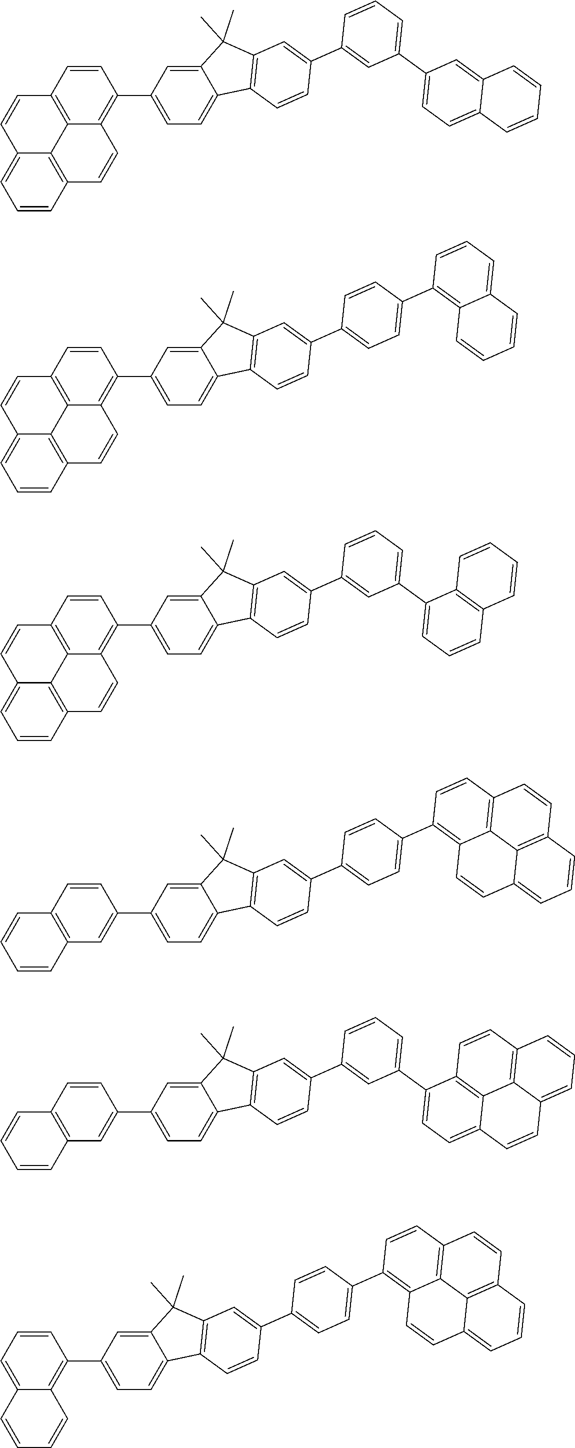

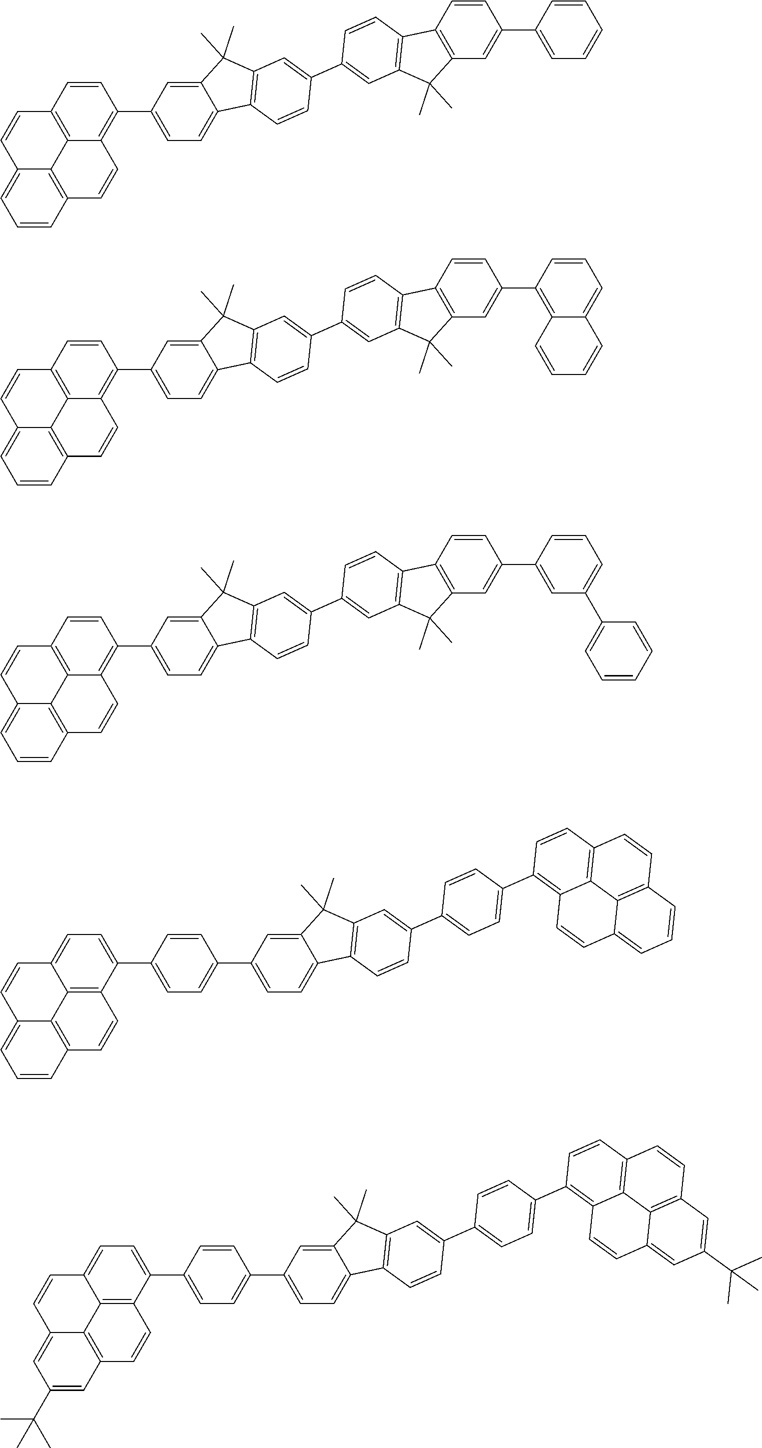

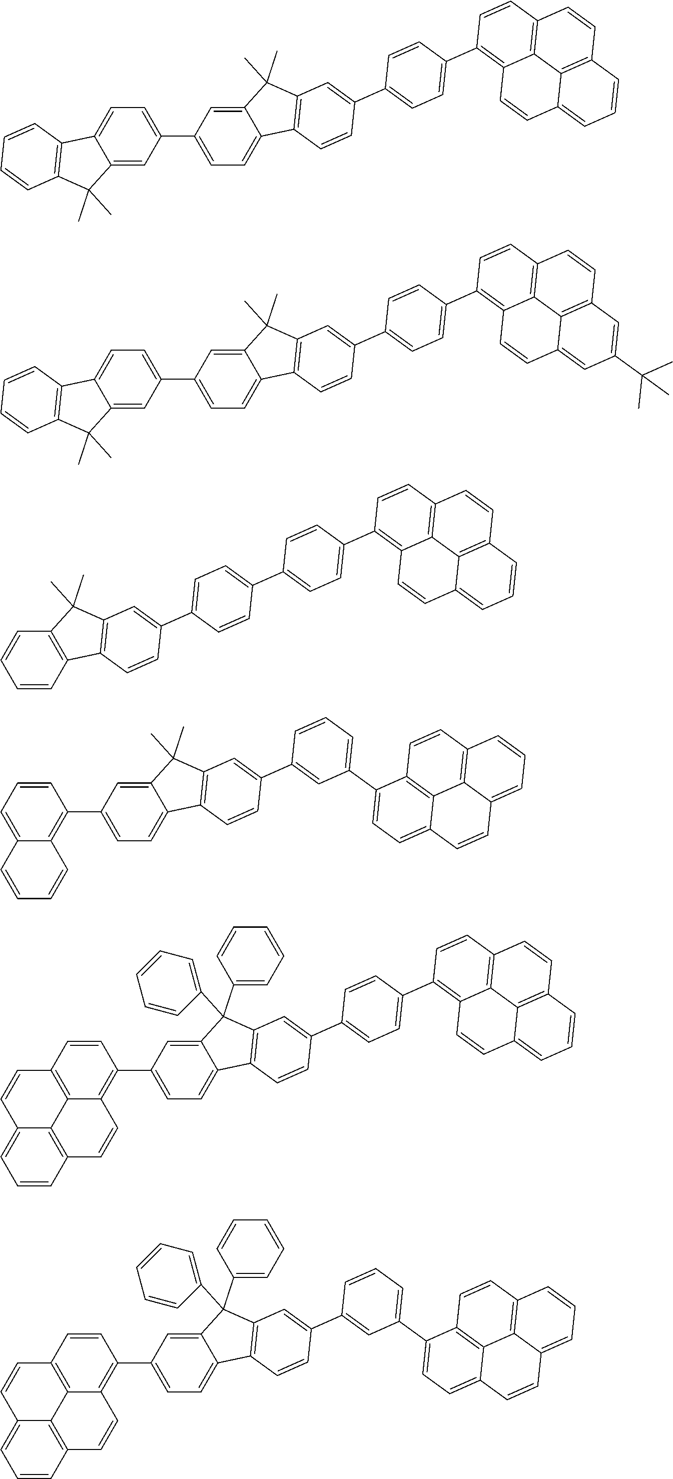

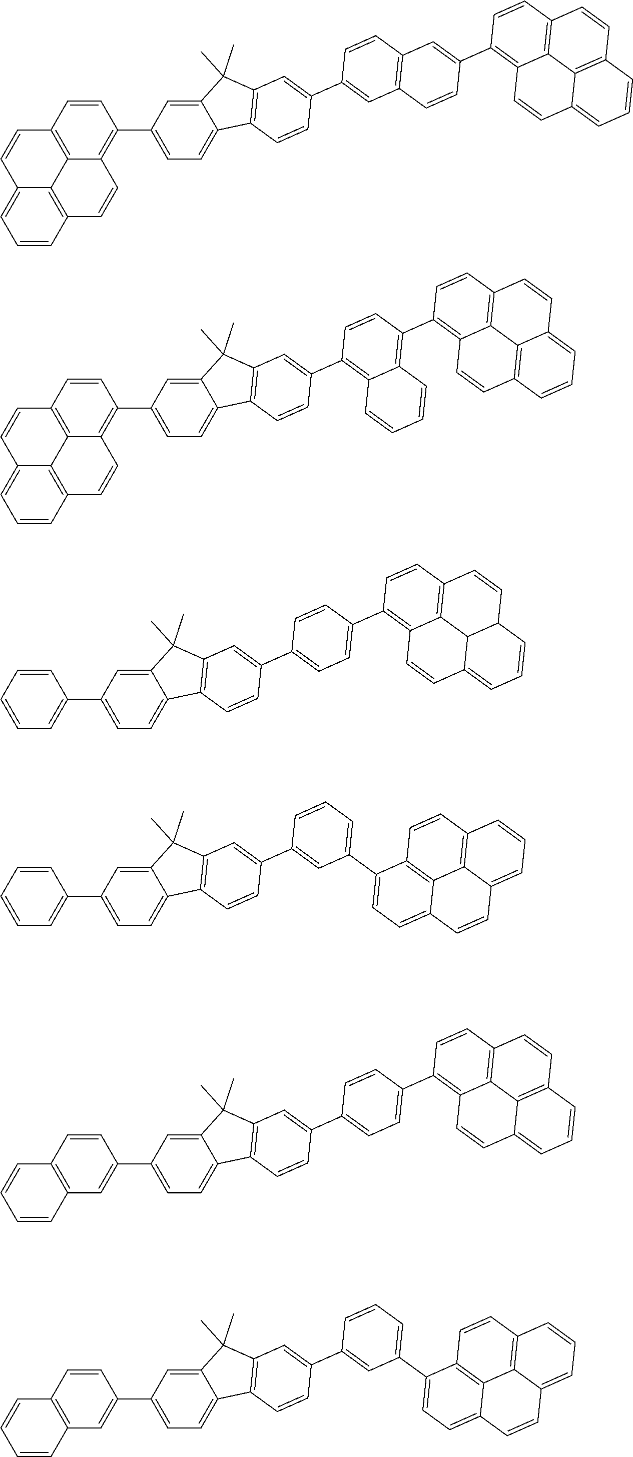

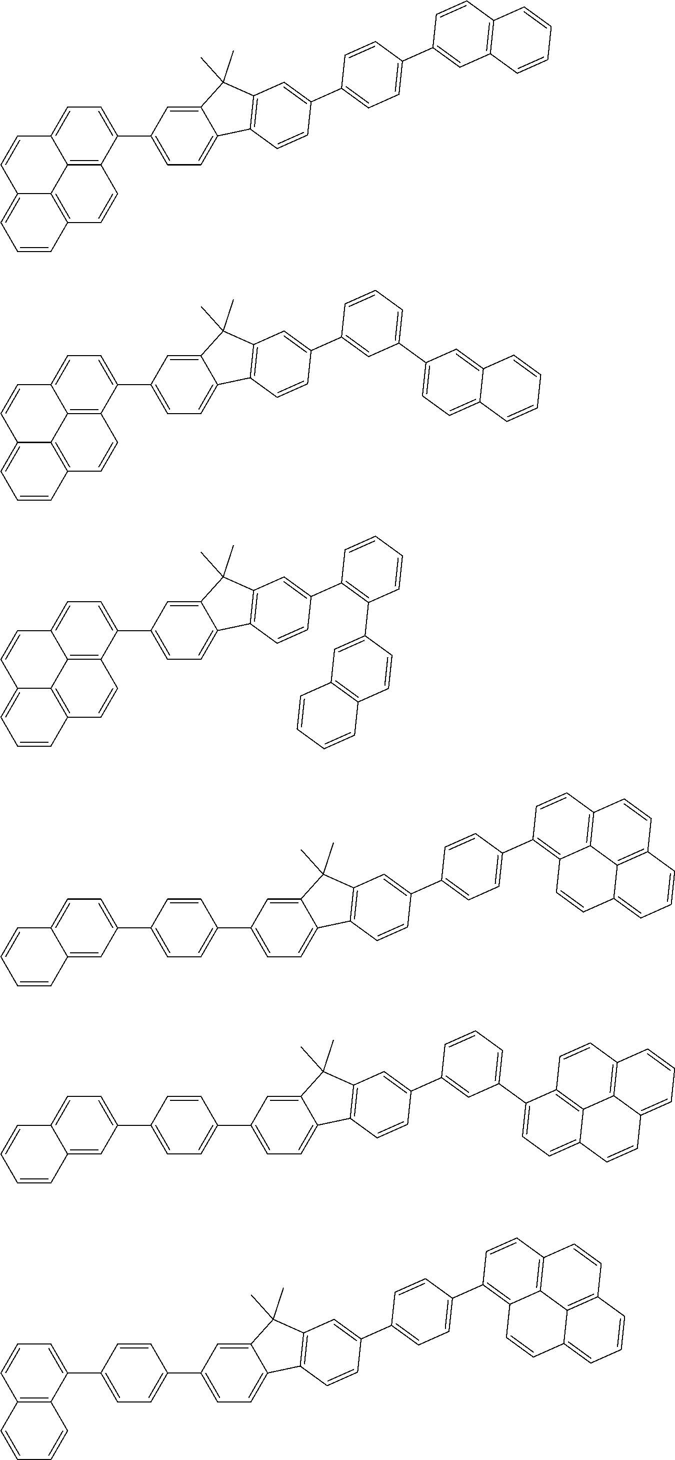

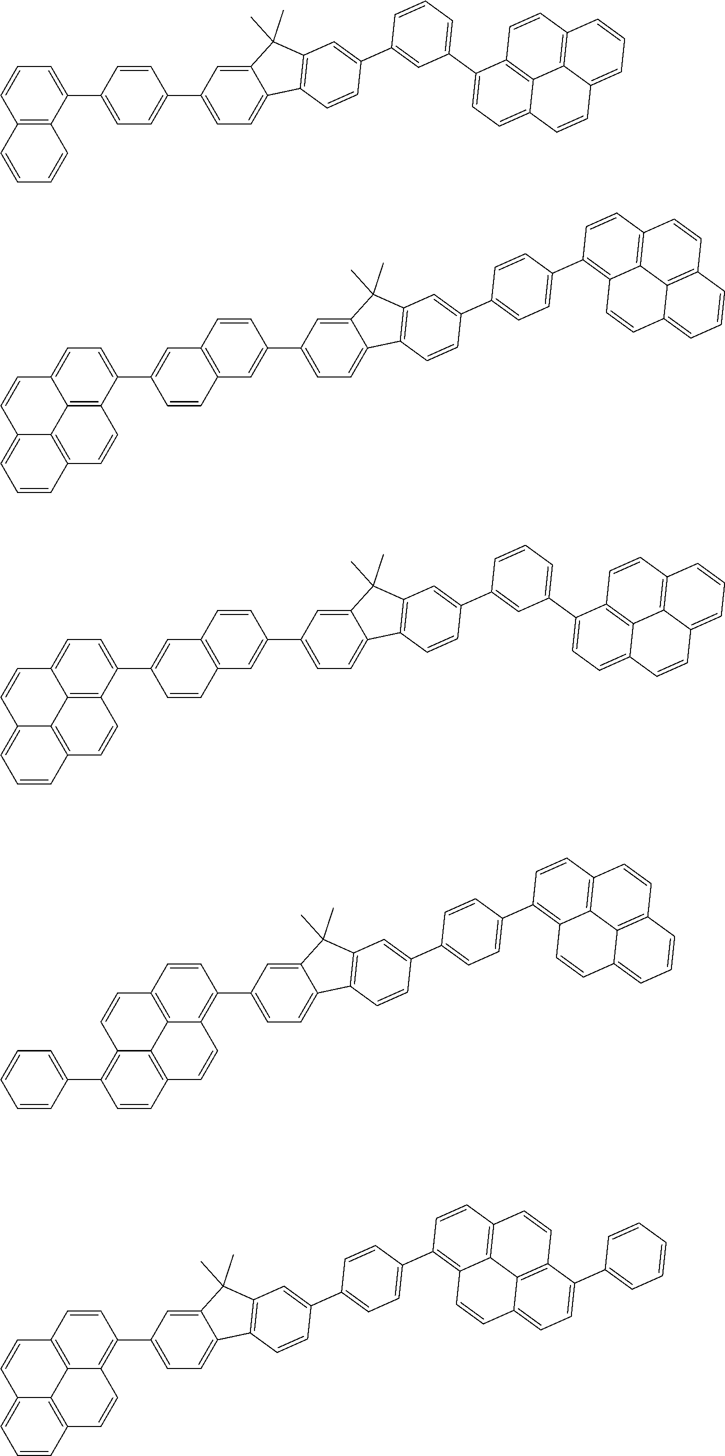

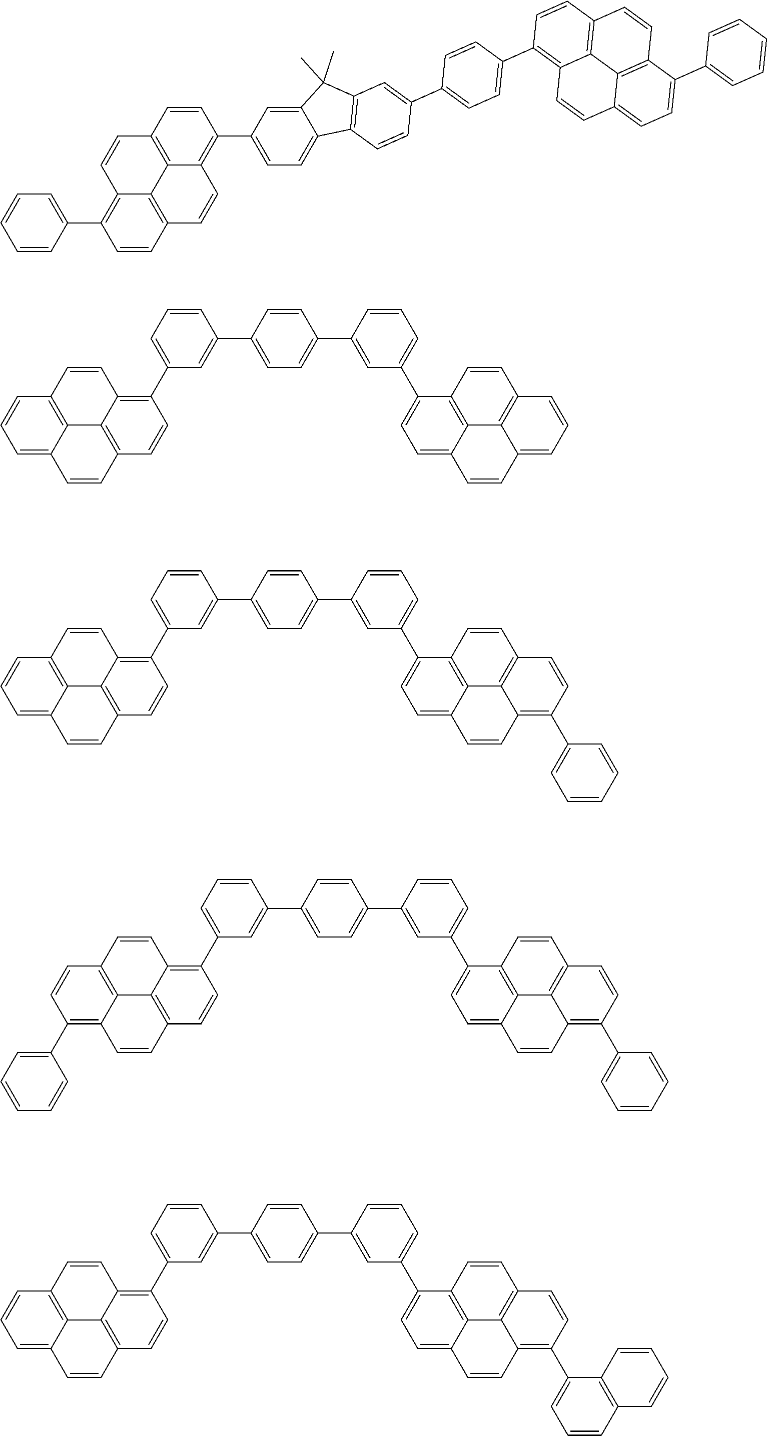

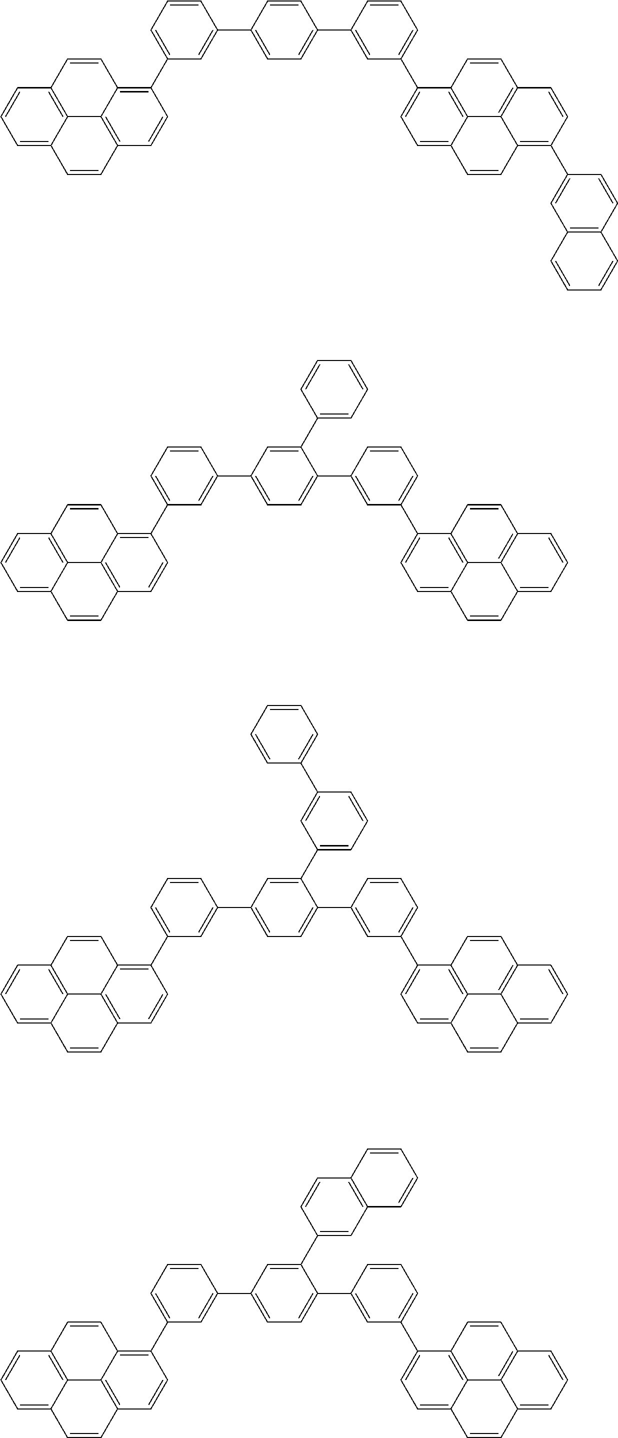

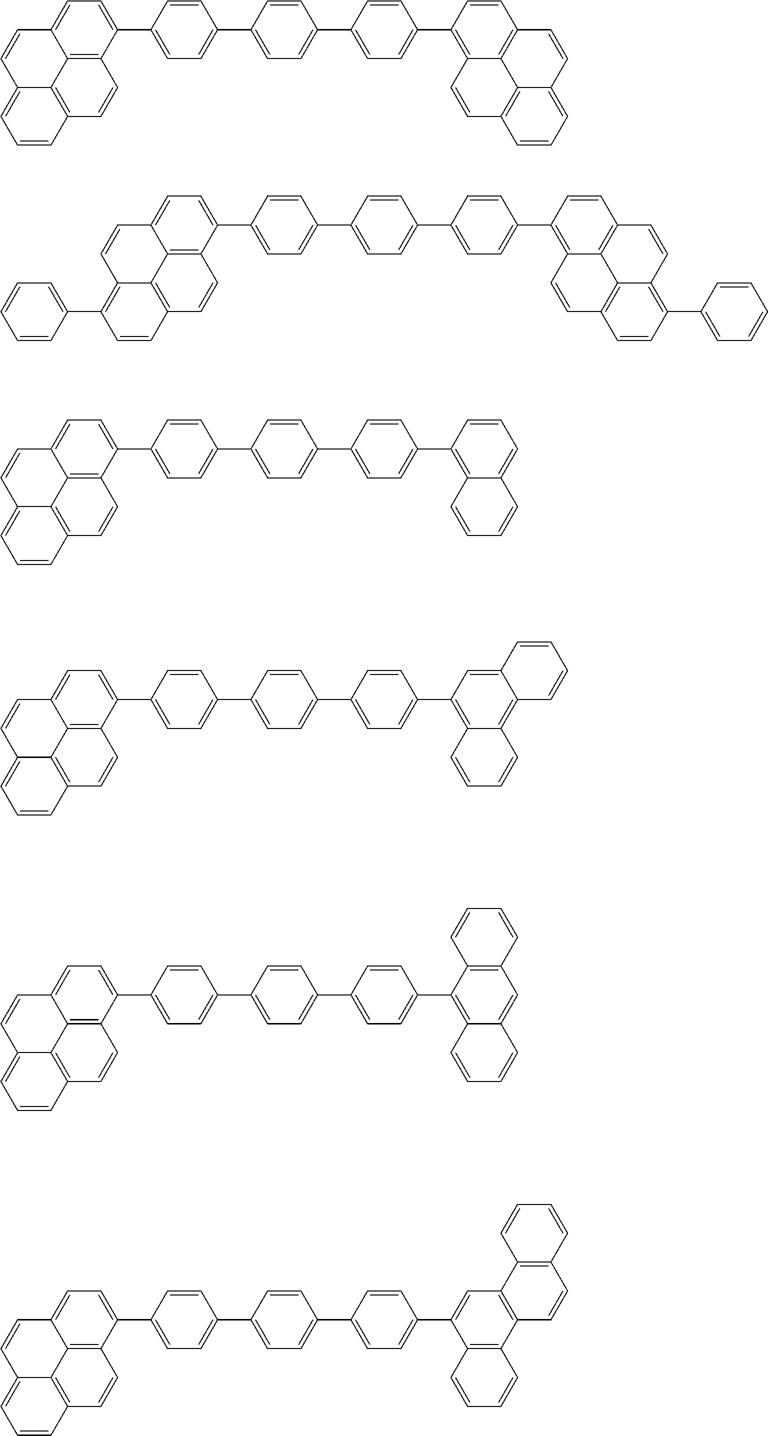

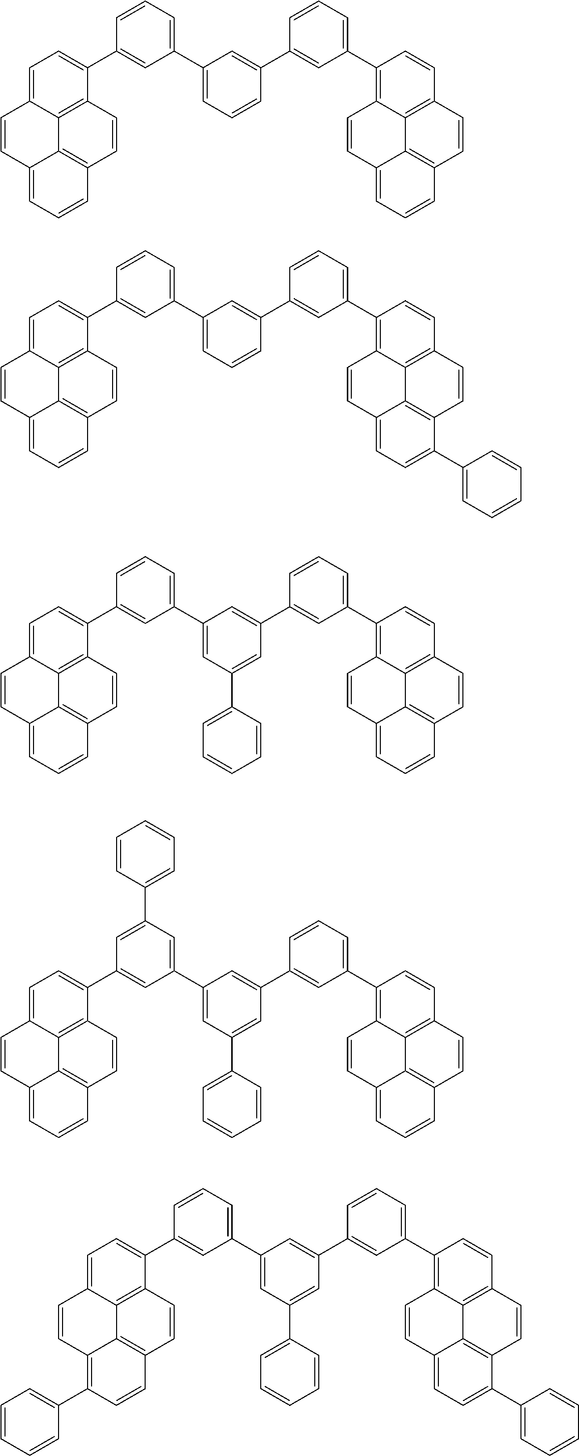

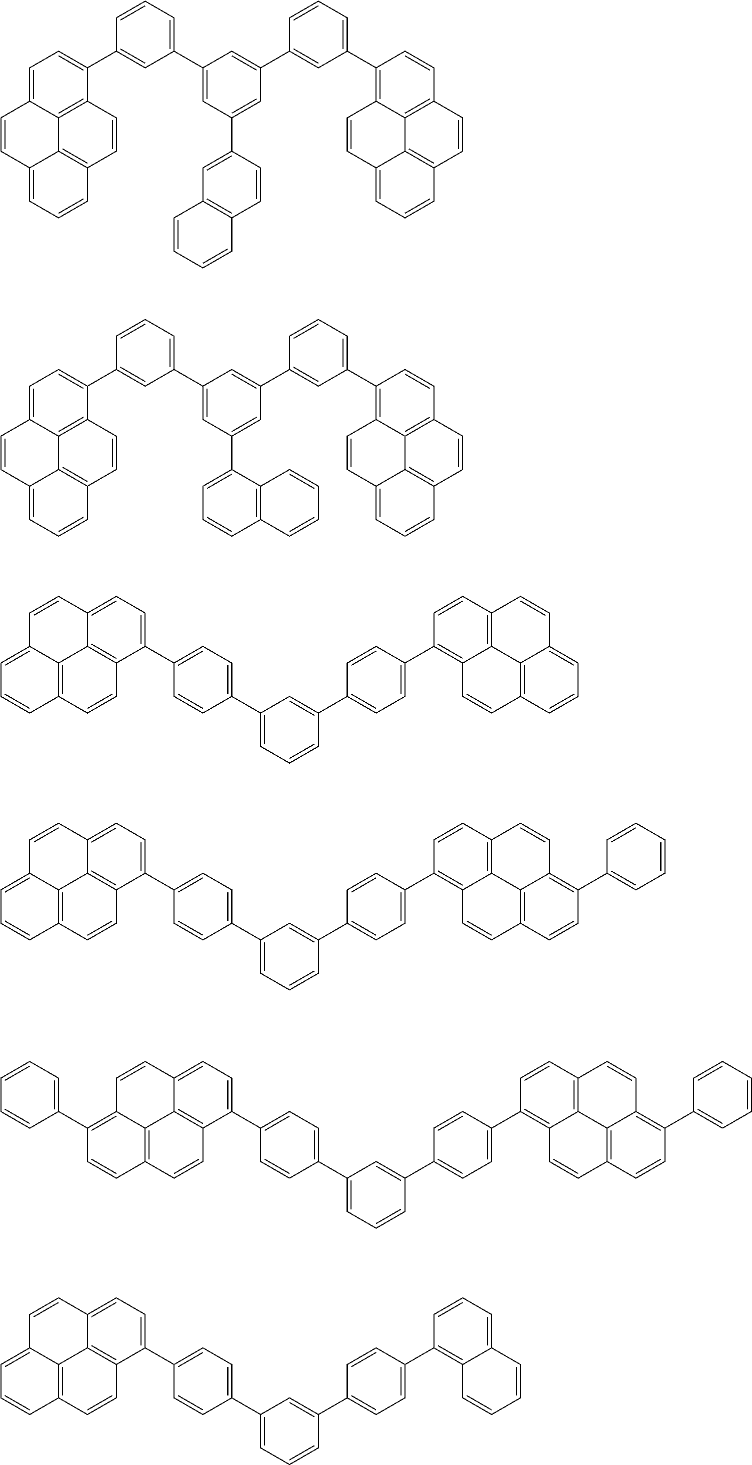

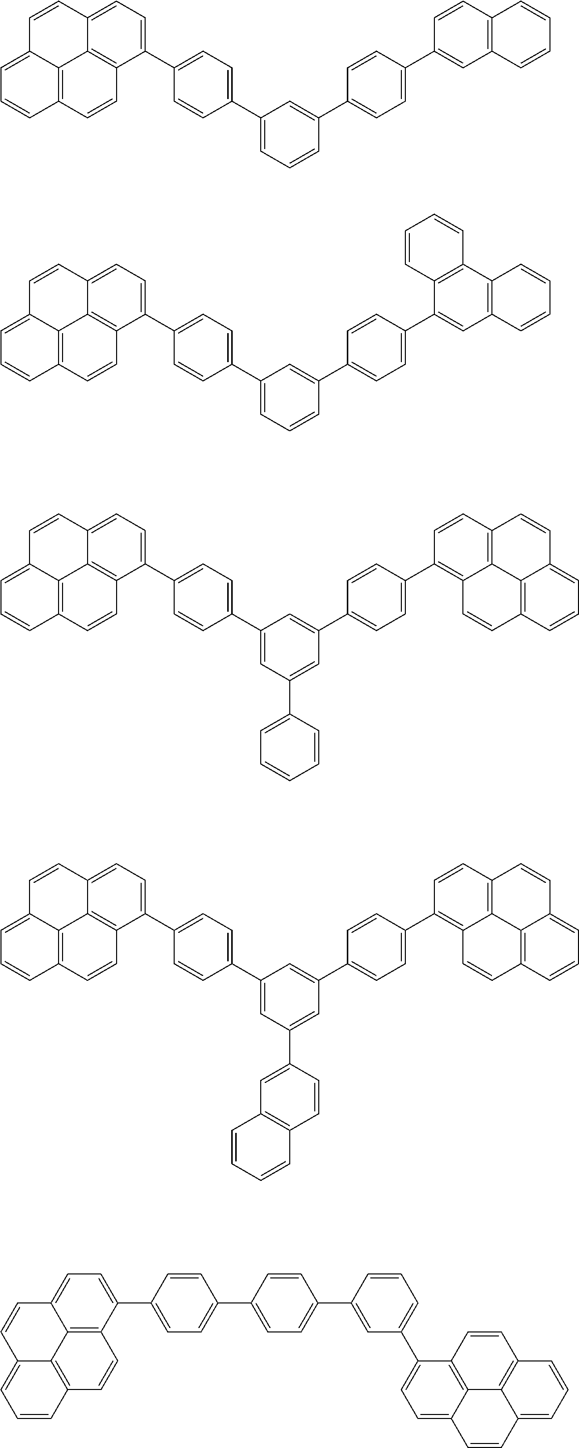

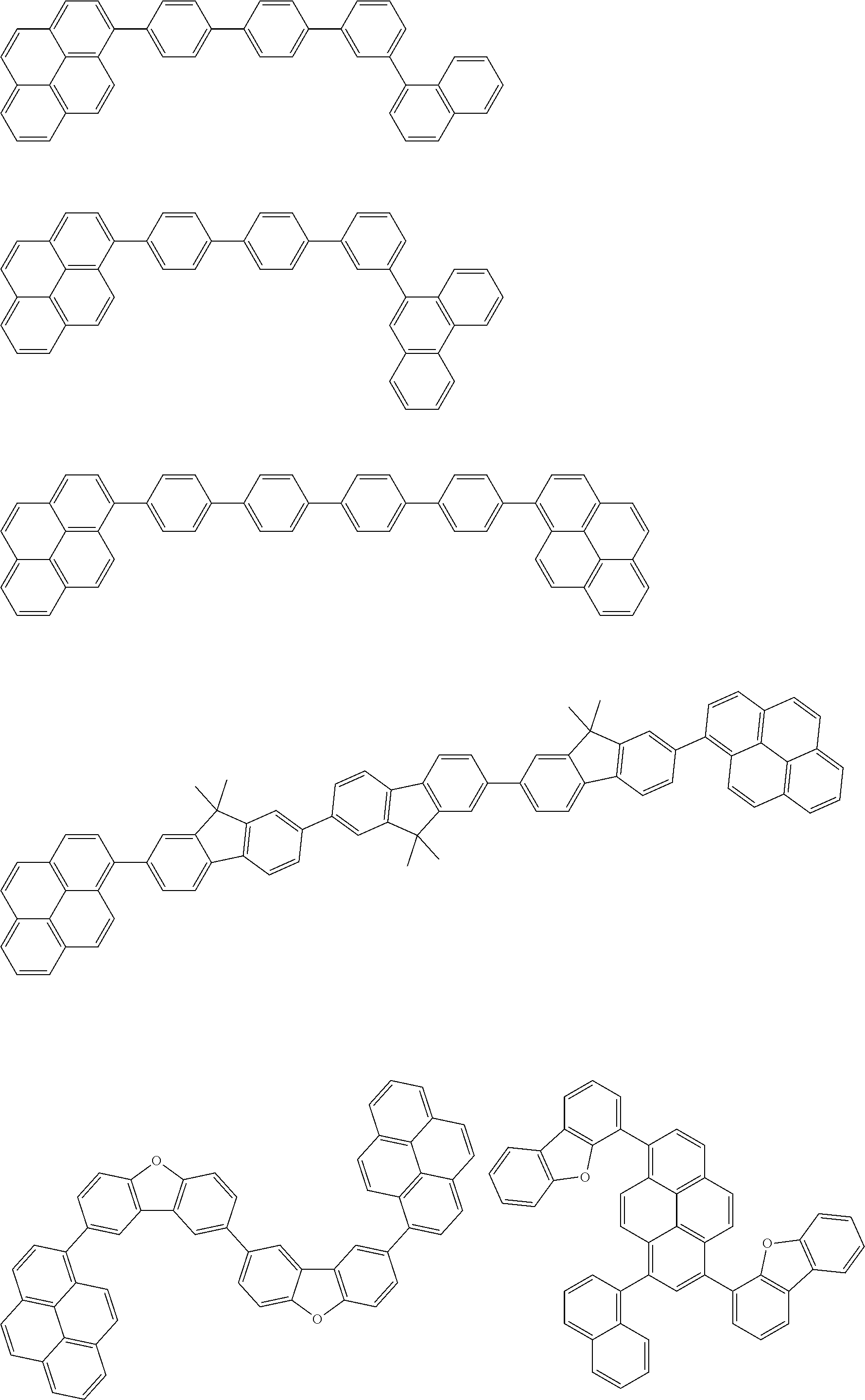

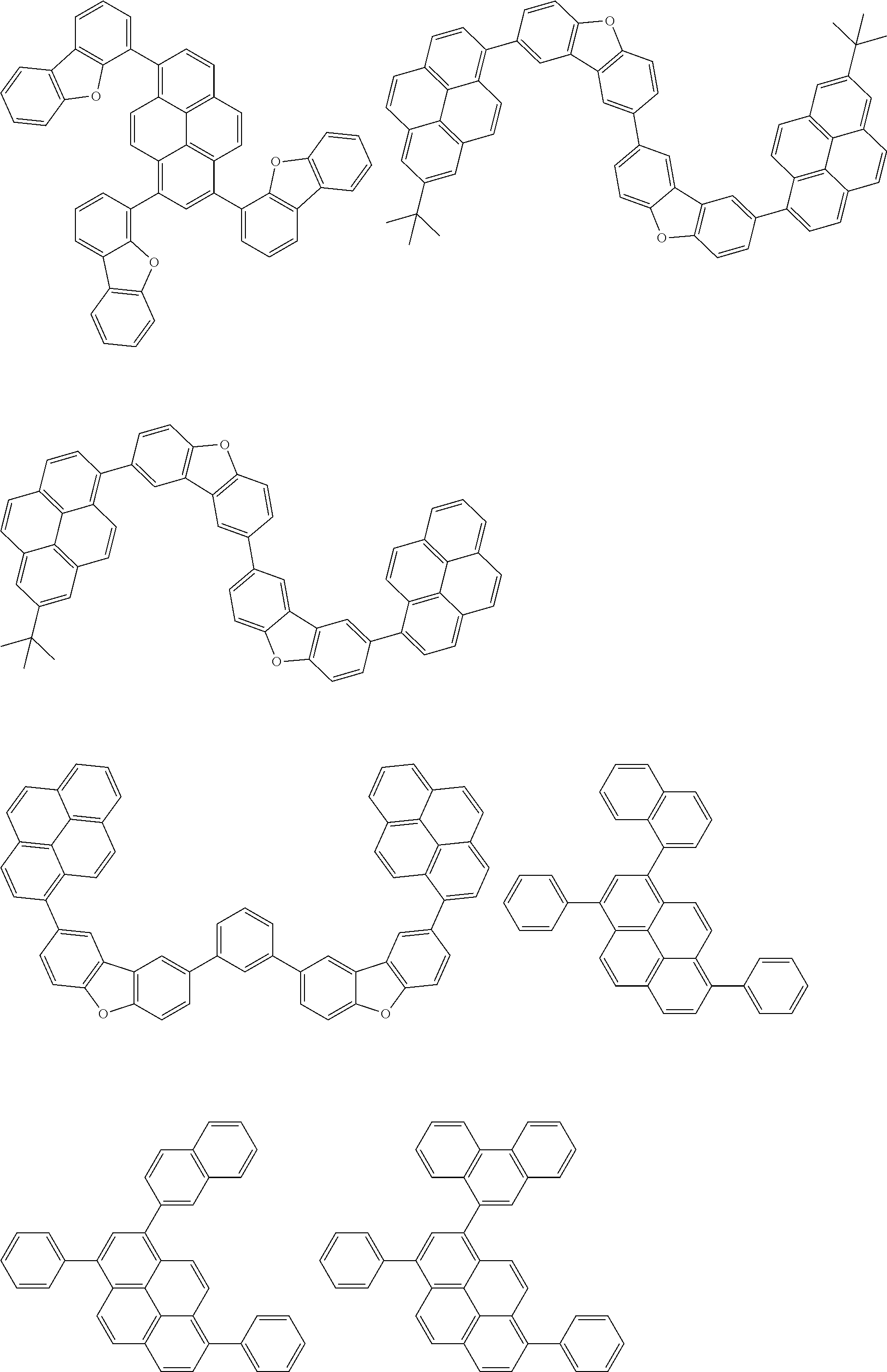

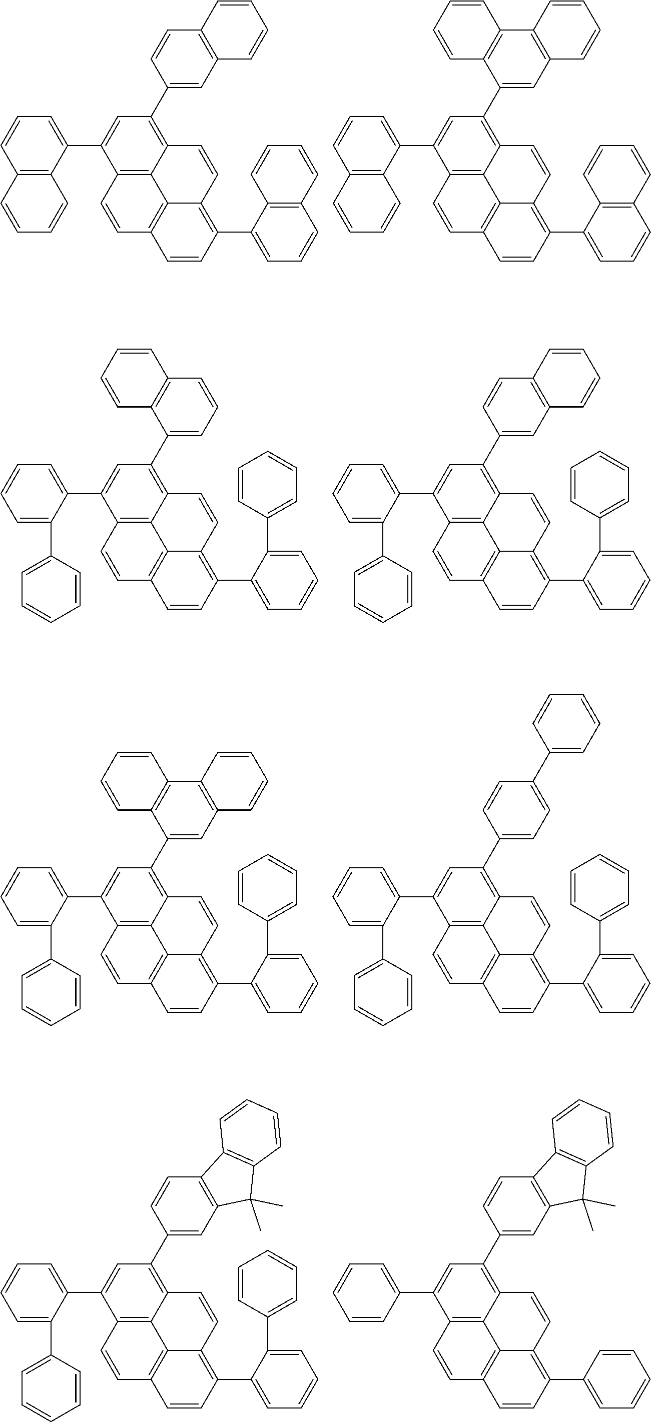

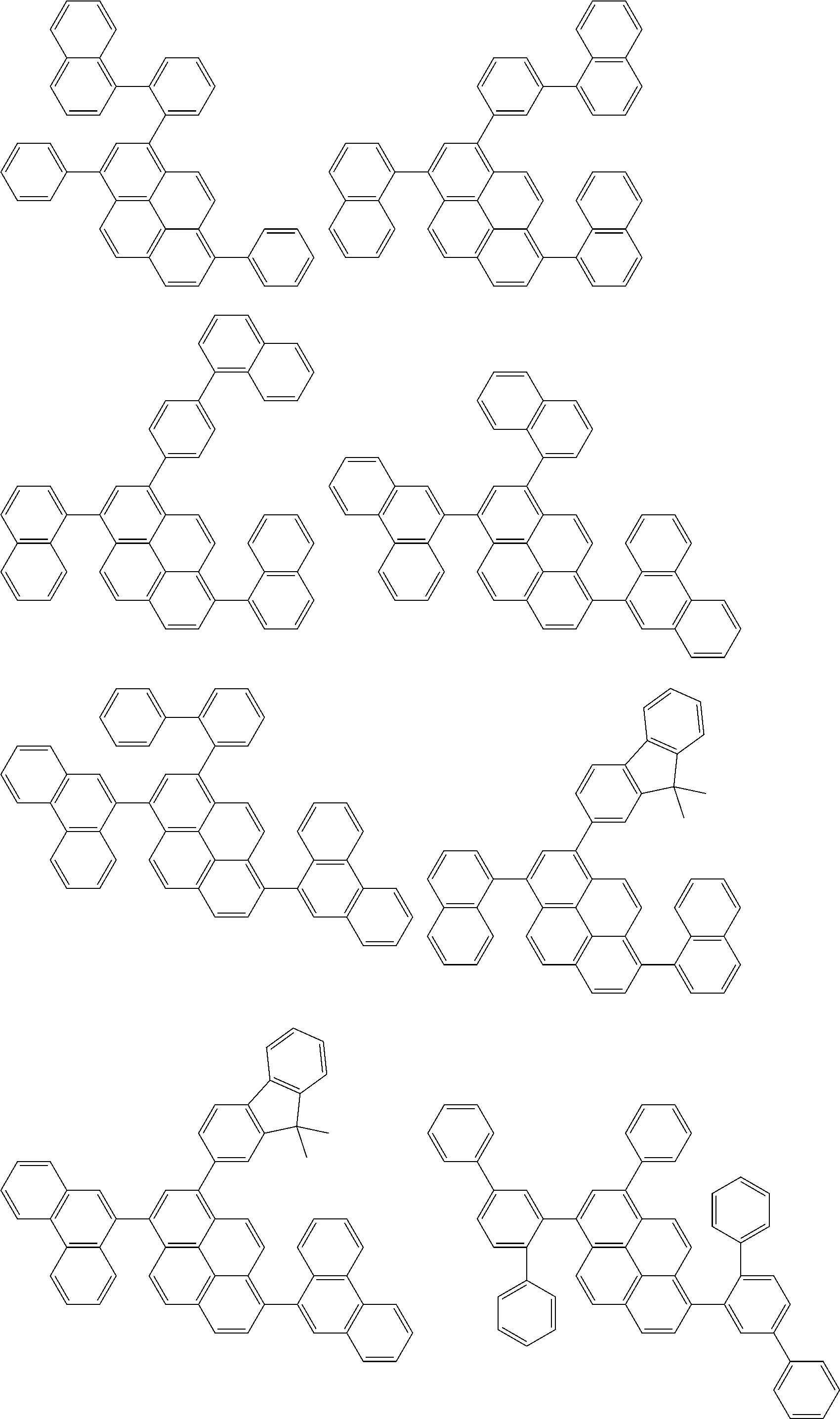

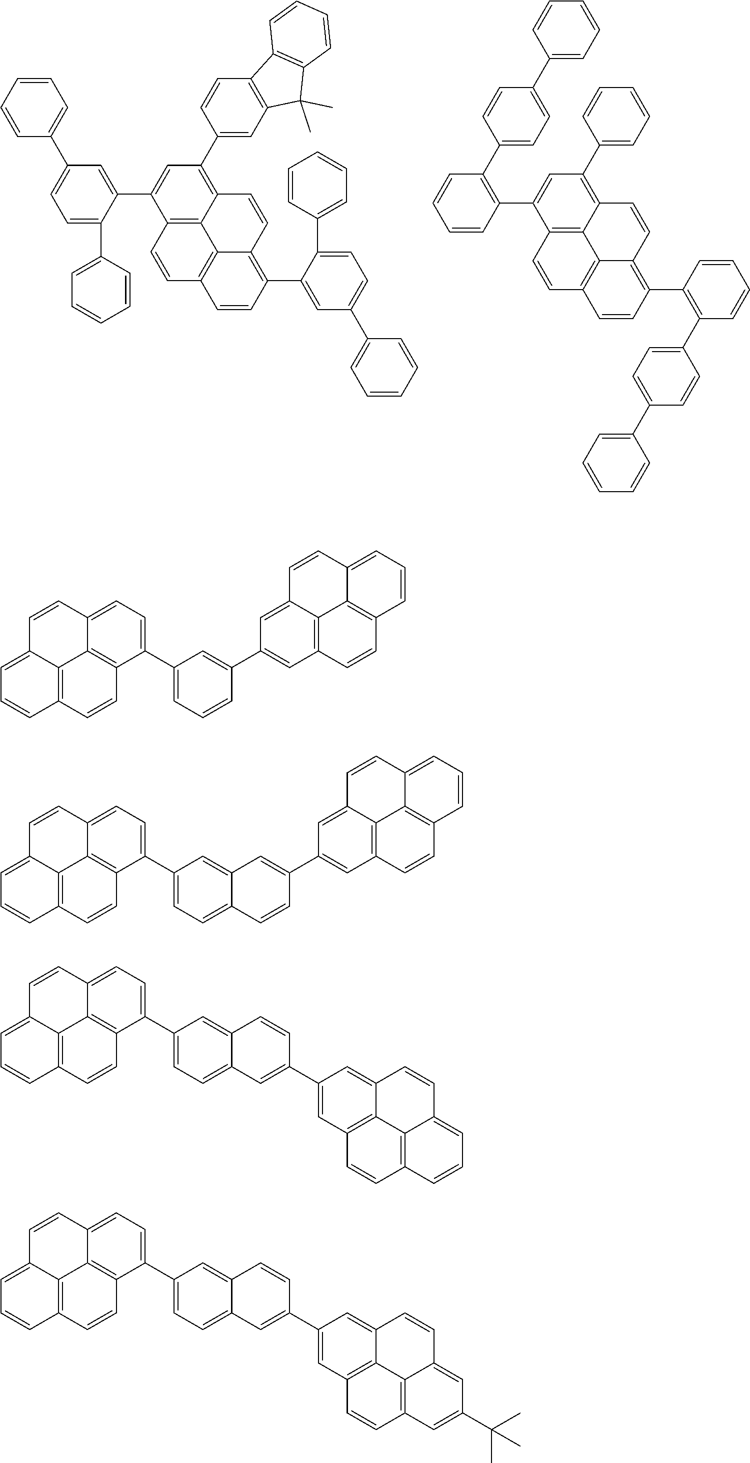

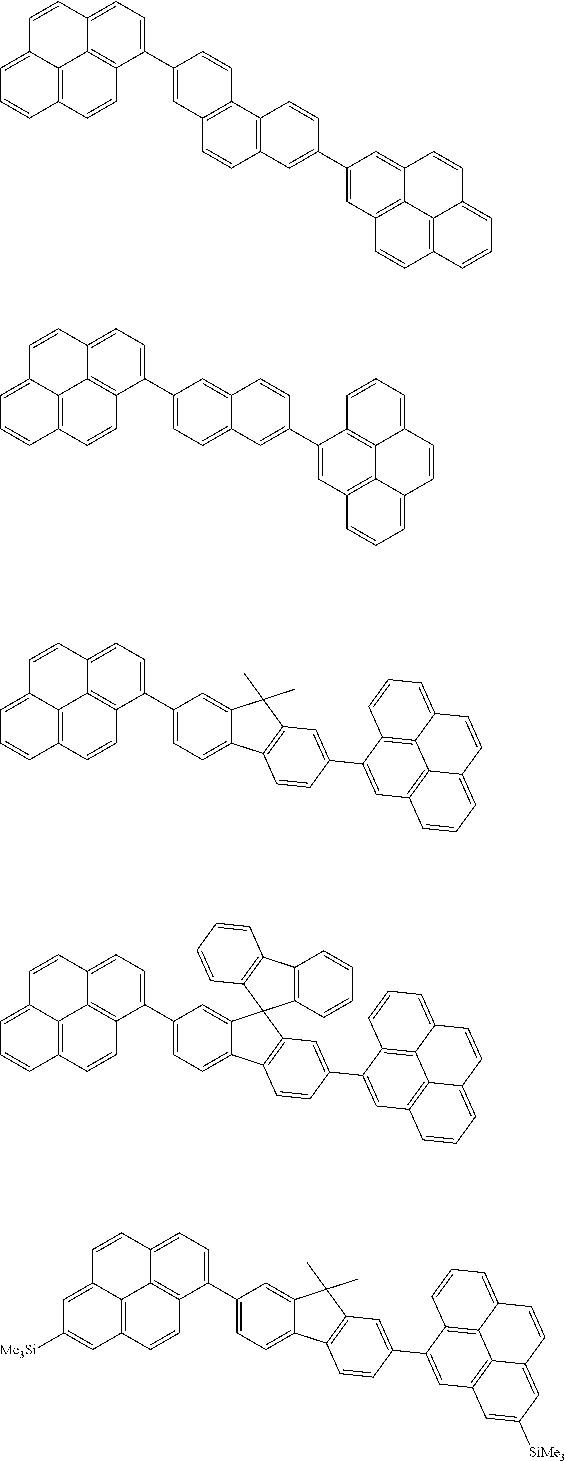

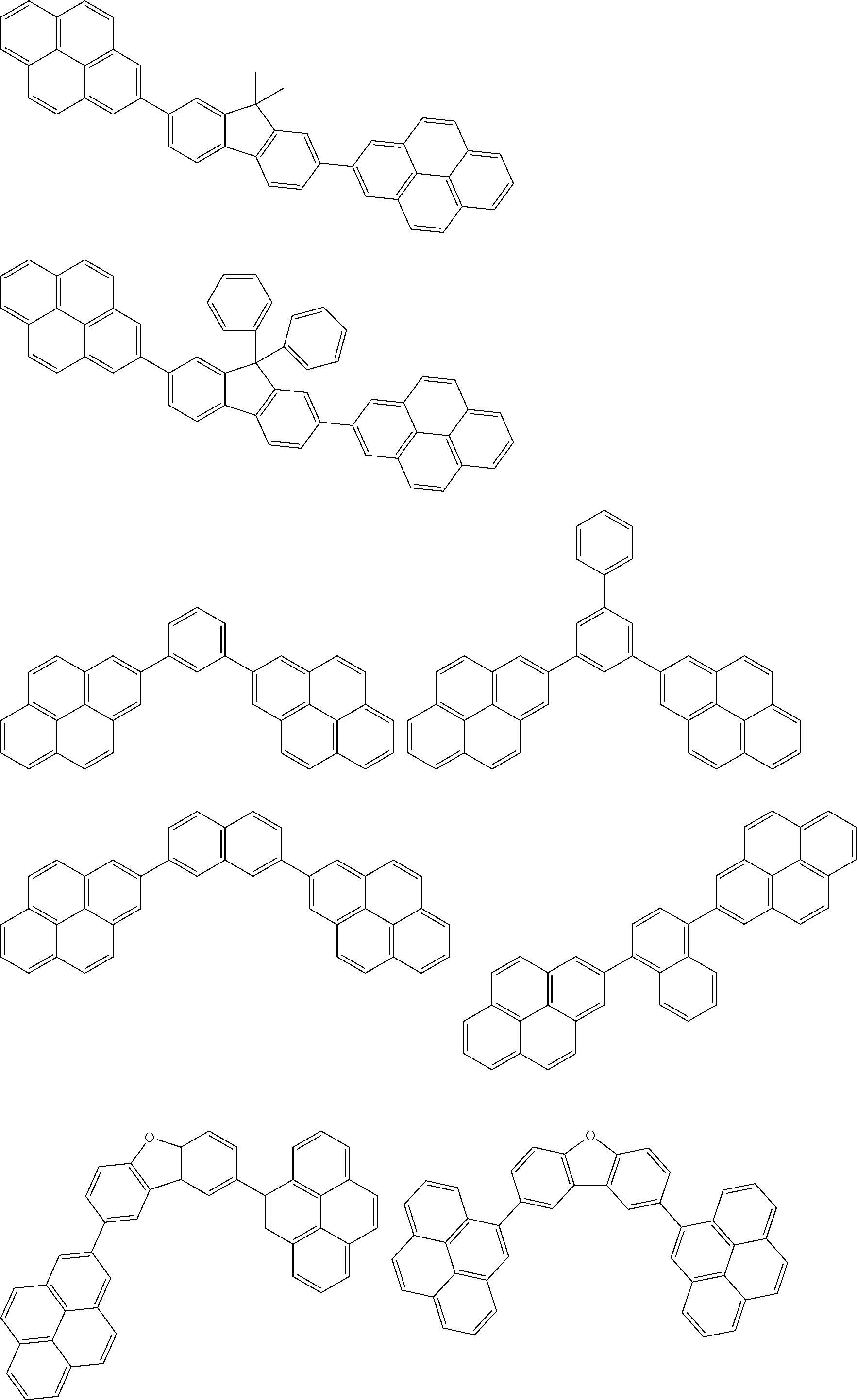

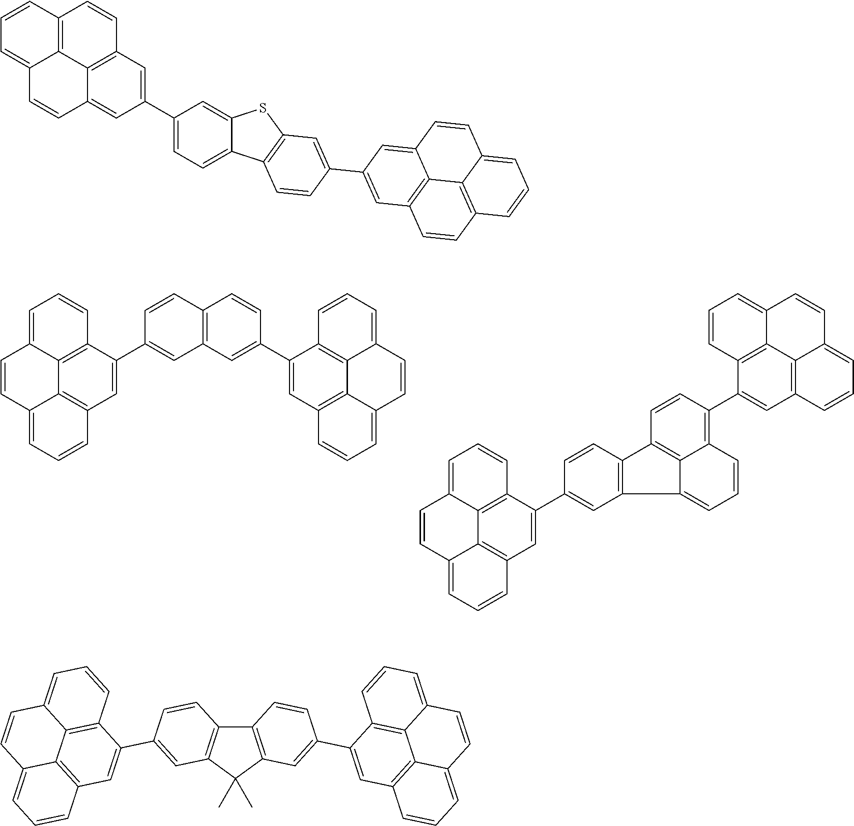

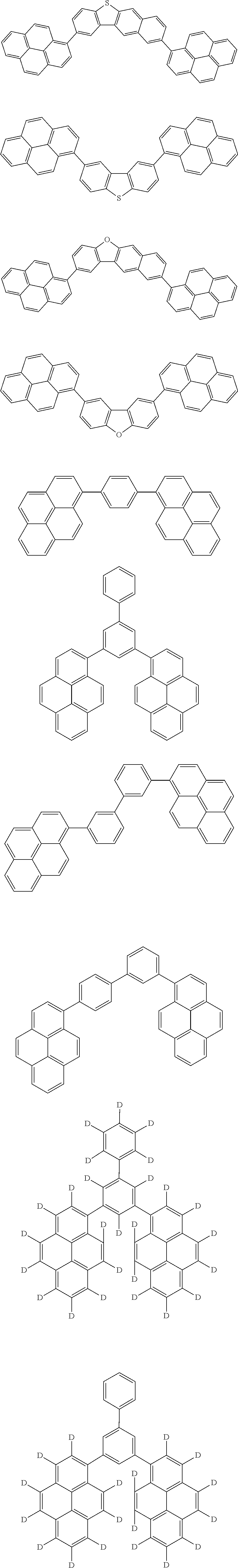

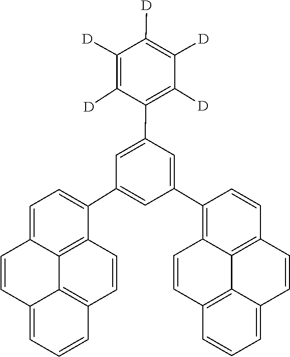 [SMILES: [2H]c1c([2H])c([2H])c(-c2cc(-c3ccc4ccc5cccc6ccc3c4c56)cc(-c3ccc4ccc5cccc6ccc3c4c56)c2)c([2H])c1[2H]]

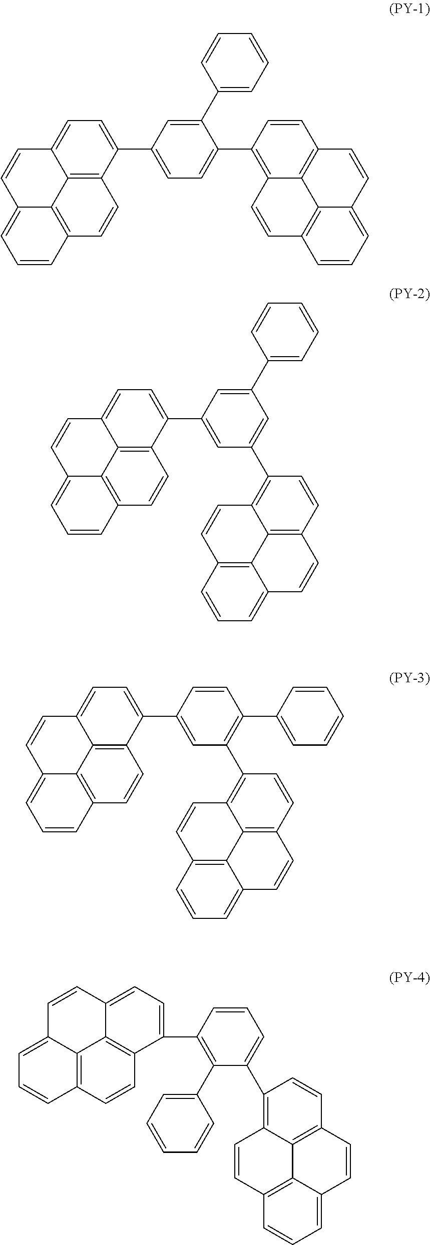

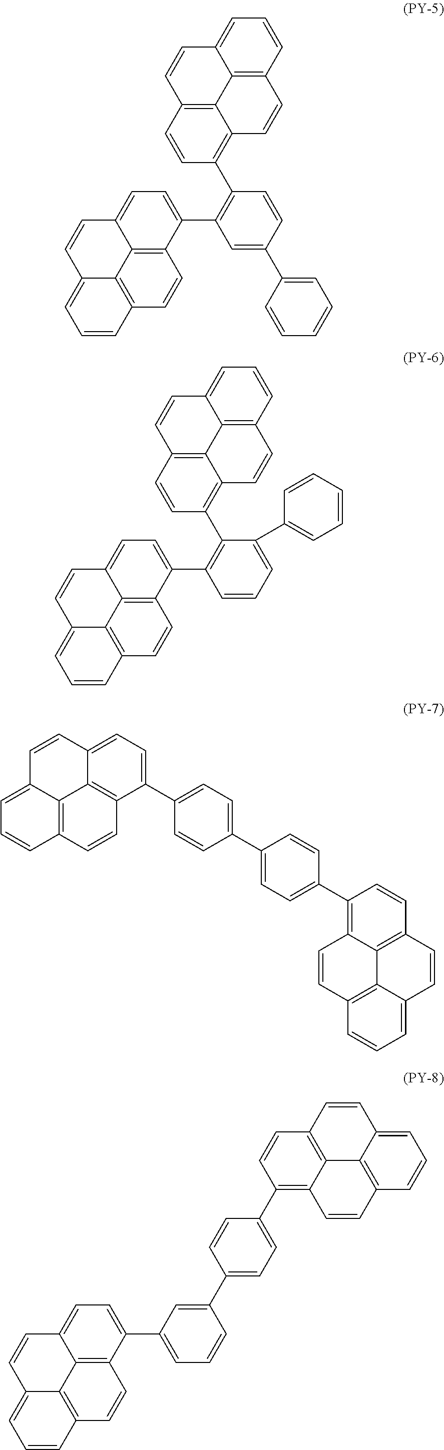

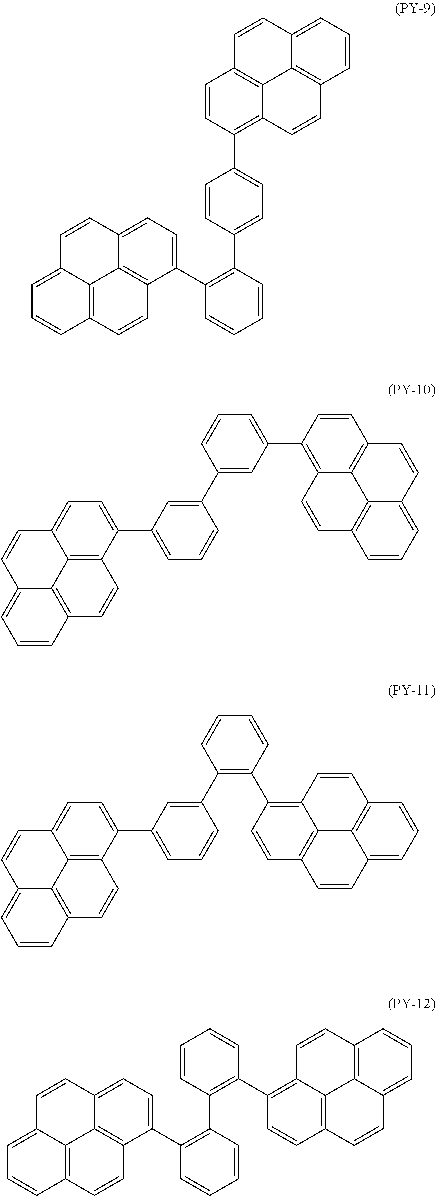

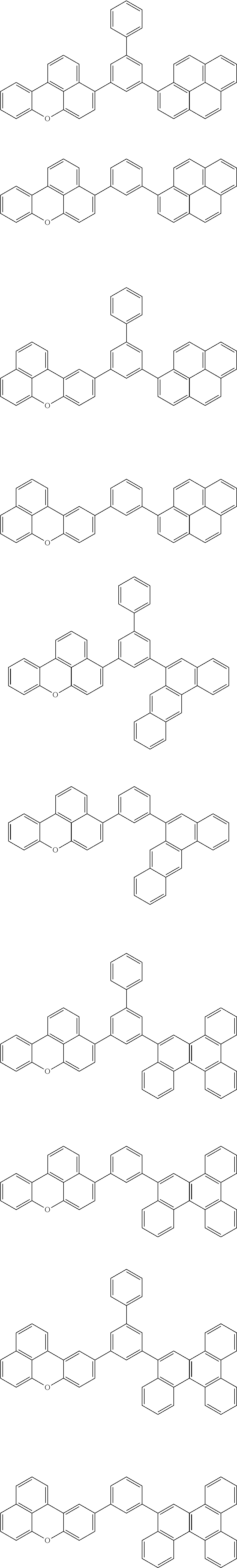

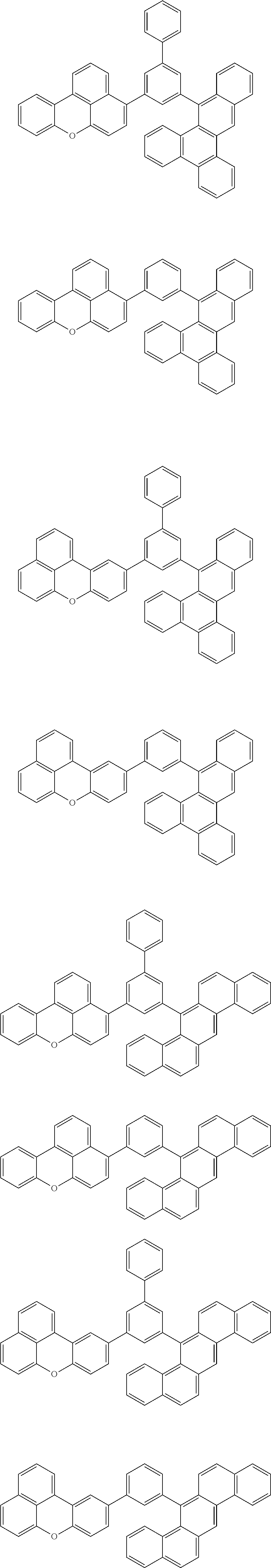

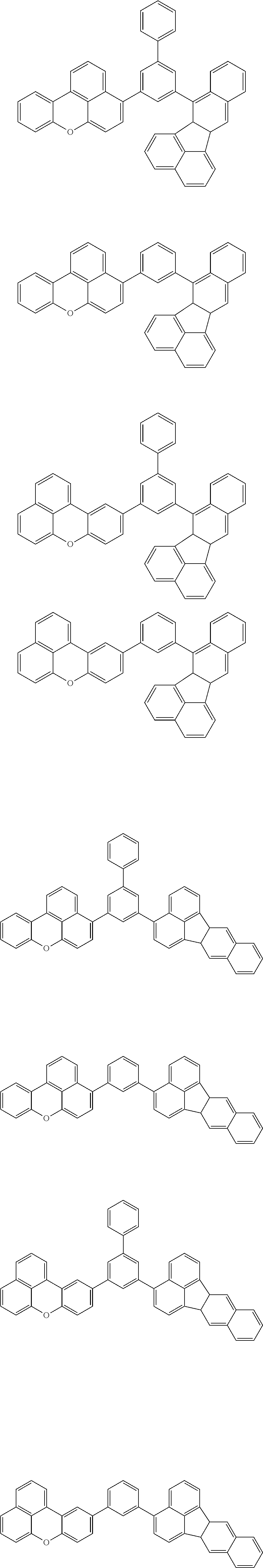

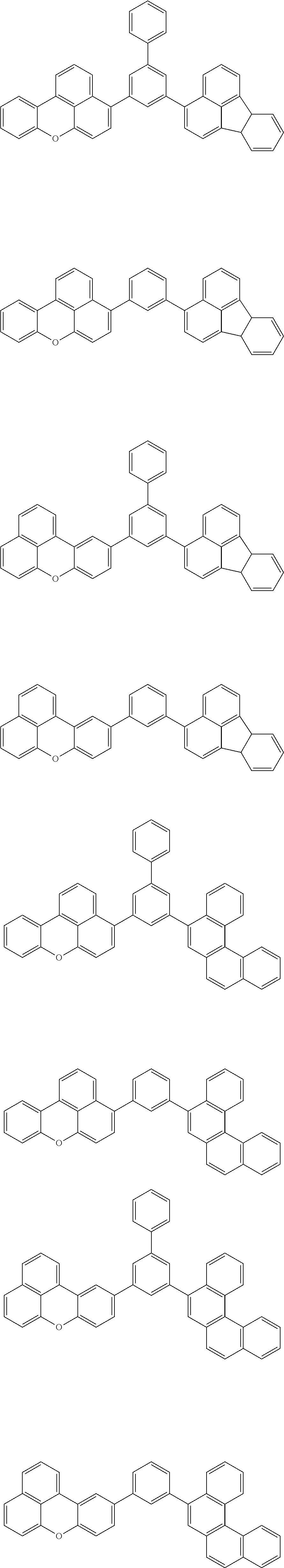

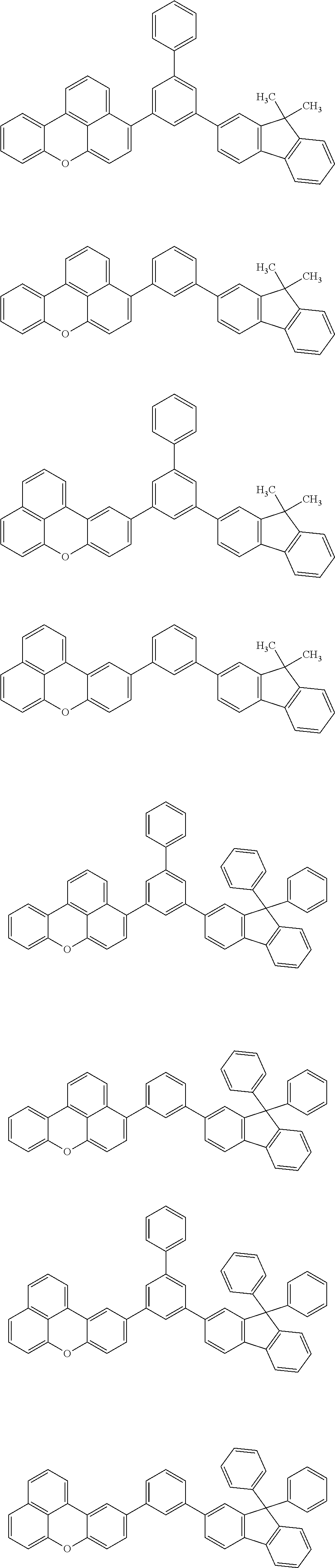

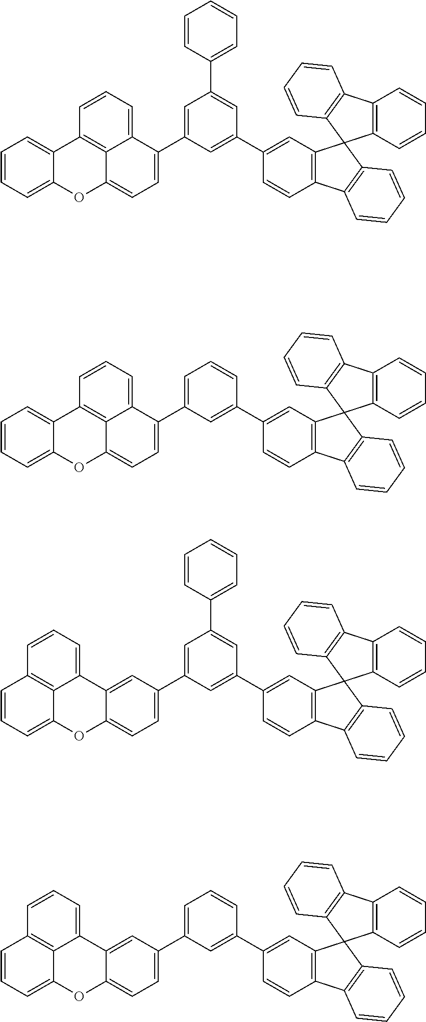 [SMILES: c1ccc(-c2cc(-c3ccc4c(c3)C3(c5ccccc5-c5ccccc53)c3ccccc3-4)cc(-c3ccc4c5c(cccc35)-c3ccccc3O4)c2)cc1 ; c1cc(-c2ccc3c(c2)C2(c4ccccc4-c4ccccc42)c2ccccc2-3)cc(-c2ccc3c4c(cccc24)-c2ccccc2O3)c1 ; c1ccc(-c2cc(-c3ccc4c(c3)-c3cccc5cccc(c35)O4)cc(-c3ccc4c(c3)C3(c5ccccc5-c5ccccc53)c3ccccc3-4)c2)cc1 ; c1cc(-c2ccc3c(c2)-c2cccc4cccc(c24)O3)cc(-c2ccc3c(c2)C2(c4ccccc4-c4ccccc42)c2ccccc2-3)c1]

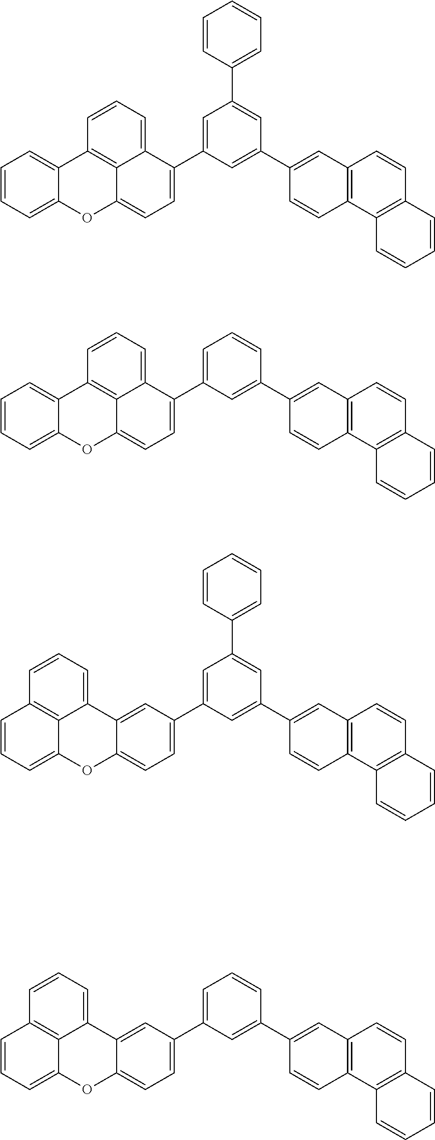 [SMILES: c1ccc(-c2cc(-c3ccc4c(ccc5ccccc54)c3)cc(-c3ccc4c5c(cccc35)-c3ccccc3O4)c2)cc1 ; c1cc(-c2ccc3c(ccc4ccccc43)c2)cc(-c2ccc3c4c(cccc24)-c2ccccc2O3)c1 ; c1ccc(-c2cc(-c3ccc4c(c3)-c3cccc5cccc(c35)O4)cc(-c3ccc4c(ccc5ccccc54)c3)c2)cc1 ; c1cc(-c2ccc3c(c2)-c2cccc4cccc(c24)O3)cc(-c2ccc3c(ccc4ccccc43)c2)c1]

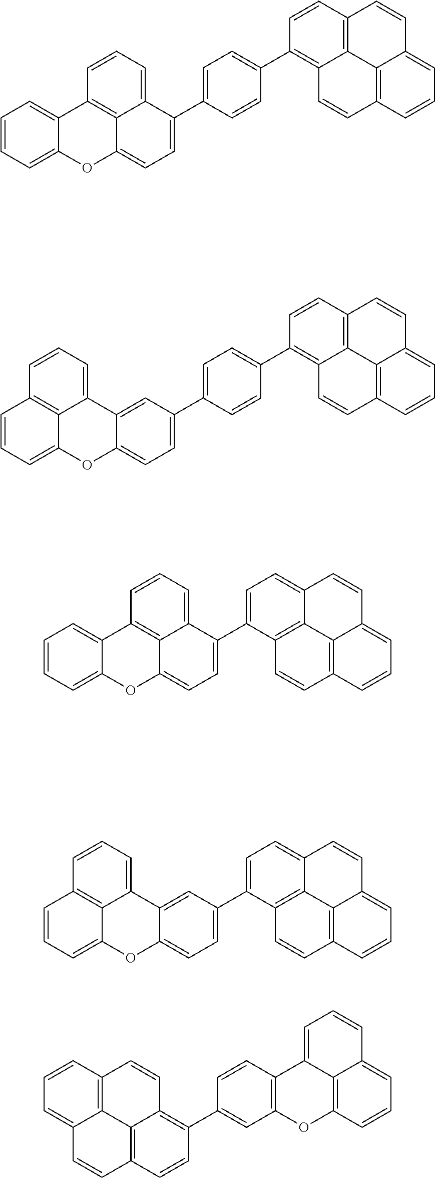 [SMILES: c1ccc2c(c1)Oc1ccc(-c3ccc(-c4ccc5ccc6cccc7ccc4c5c67)cc3)c3cccc-2c13 ; c1cc2c3c(cccc3c1)-c1cc(-c3ccc(-c4ccc5ccc6cccc7ccc4c5c67)cc3)ccc1O2 ; c1ccc2c(c1)Oc1ccc(-c3ccc4ccc5cccc6ccc3c4c56)c3cccc-2c13 ; c1cc2c3c(cccc3c1)-c1cc(-c3ccc4ccc5cccc6ccc3c4c56)ccc1O2 ; c1cc2c3c(cccc3c1)-c1ccc(-c3ccc4ccc5cccc6ccc3c4c56)cc1O2]

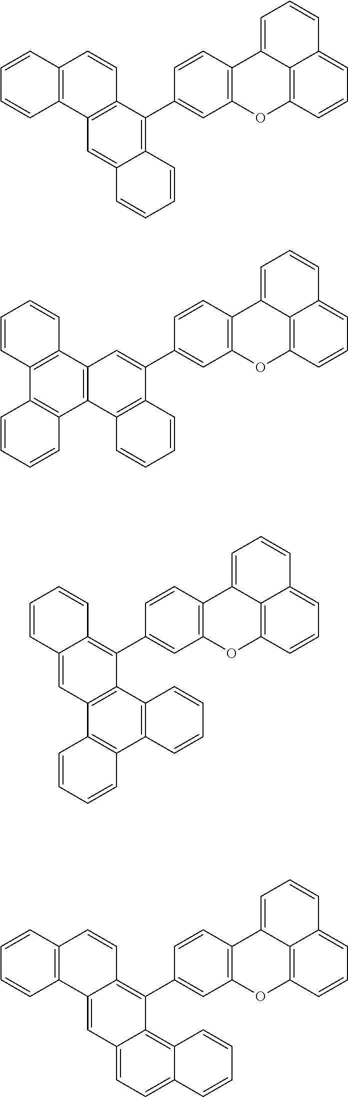 [SMILES: c1ccc2c(-c3ccc4c(c3)Oc3cccc5cccc-4c35)c3ccc4ccccc4c3cc2c1 ; c1cc2c3c(cccc3c1)-c1ccc(-c3cc4c5ccccc5c5ccccc5c4c4ccccc34)cc1O2 ; c1ccc2c(-c3ccc4c(c3)Oc3cccc5cccc-4c35)c3c4ccccc4c4ccccc4c3cc2c1 ; c1cc2c3c(cccc3c1)-c1ccc(-c3c4ccc5ccccc5c4cc4ccc5ccccc5c34)cc1O2]

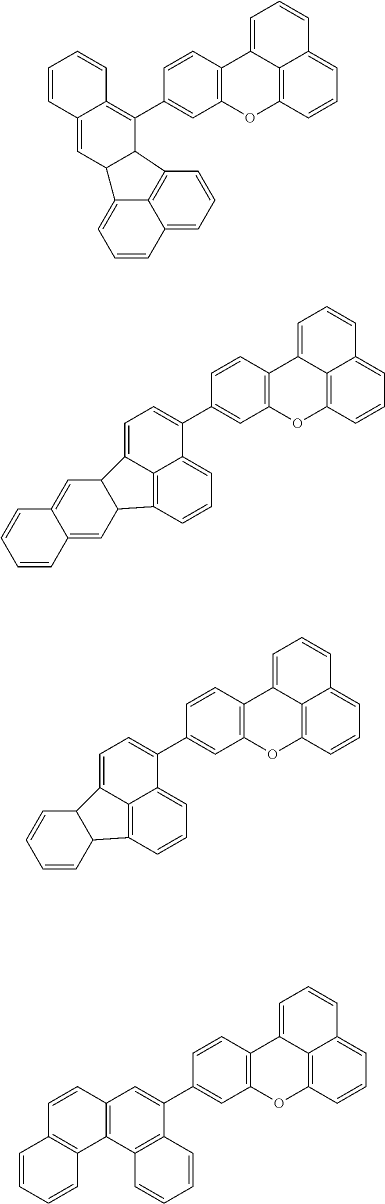 [SMILES: C1=c2ccccc2=C(c2ccc3c(c2)Oc2cccc4cccc-3c24)C2c3cccc4cccc(c34)C12 ; C1=c2ccccc2=CC2c3ccc(-c4ccc5c(c4)Oc4cccc6cccc-5c46)c4cccc(c34)C12 ; C1=CC2c3cccc4c(-c5ccc6c(c5)Oc5cccc7cccc-6c57)ccc(c34)C2C=C1 ; c1cc2c3c(cccc3c1)-c1ccc(-c3cc4ccc5ccccc5c4c4ccccc34)cc1O2]

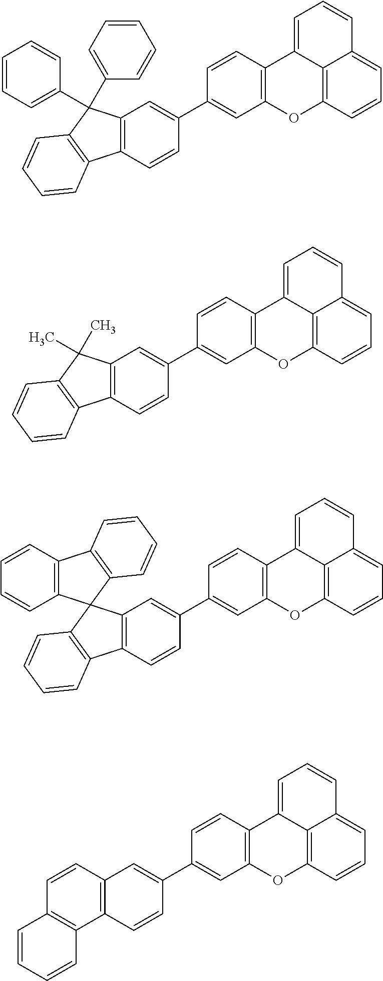 [SMILES: c1ccc(C2(c3ccccc3)c3ccccc3-c3ccc(-c4ccc5c(c4)Oc4cccc6cccc-5c46)cc32)cc1 ; CC1(C)c2ccccc2-c2ccc(-c3ccc4c(c3)Oc3cccc5cccc-4c35)cc21 ; c1ccc2c(c1)-c1ccccc1C21c2ccccc2-c2ccc(-c3ccc4c(c3)Oc3cccc5cccc-4c35)cc21 ; c1cc2c3c(cccc3c1)-c1ccc(-c3ccc4c(ccc5ccccc54)c3)cc1O2]

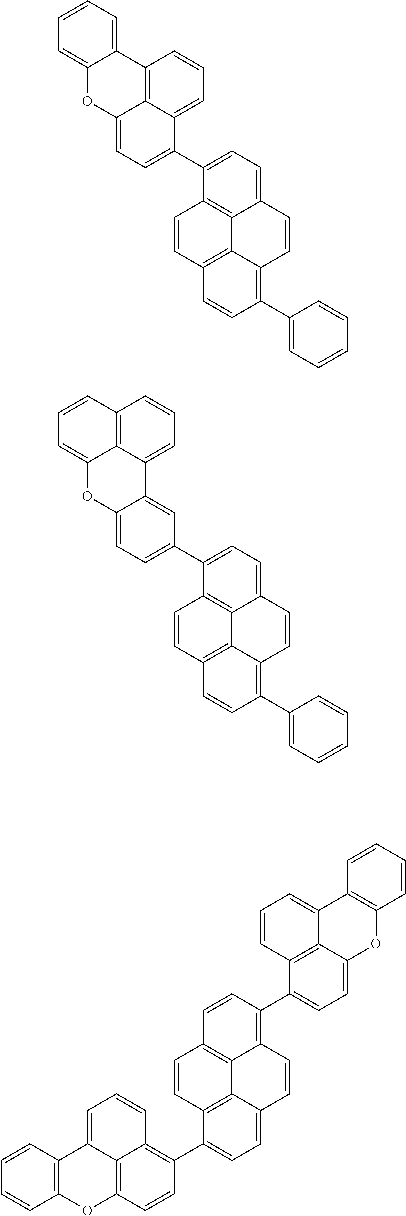 [SMILES: c1ccc(-c2ccc3ccc4c(-c5ccc6c7c(cccc57)-c5ccccc5O6)ccc5ccc2c3c54)cc1 ; c1ccc(-c2ccc3ccc4c(-c5ccc6c(c5)-c5cccc7cccc(c57)O6)ccc5ccc2c3c54)cc1 ; c1ccc2c(c1)Oc1ccc(-c3ccc4ccc5c(-c6ccc7c8c(cccc68)-c6ccccc6O7)ccc6ccc3c4c65)c3cccc-2c13]

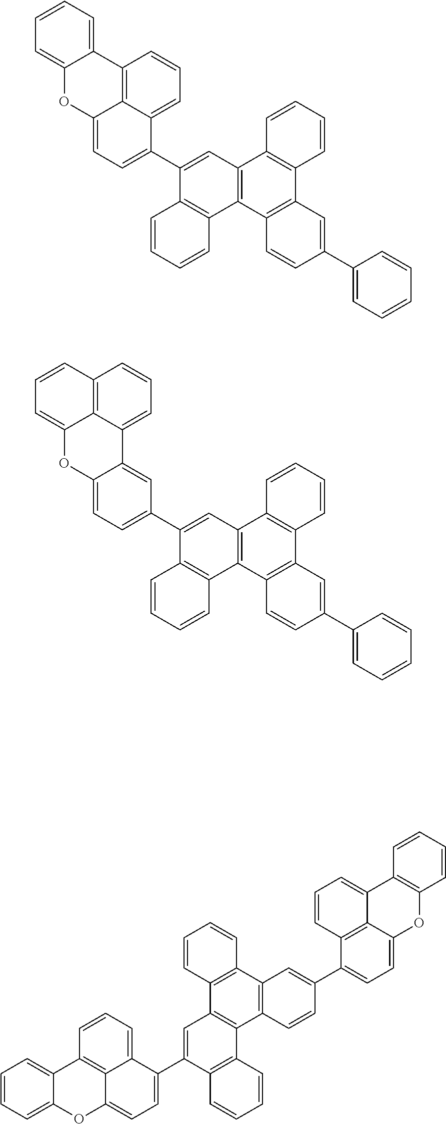 [SMILES: c1ccc(-c2ccc3c(c2)c2ccccc2c2cc(-c4ccc5c6c(cccc46)-c4ccccc4O5)c4ccccc4c32)cc1 ; c1ccc(-c2ccc3c(c2)c2ccccc2c2cc(-c4ccc5c(c4)-c4cccc6cccc(c46)O5)c4ccccc4c32)cc1 ; c1ccc2c(c1)Oc1ccc(-c3ccc4c(c3)c3ccccc3c3cc(-c5ccc6c7c(cccc57)-c5ccccc5O6)c5ccccc5c43)c3cccc-2c13]

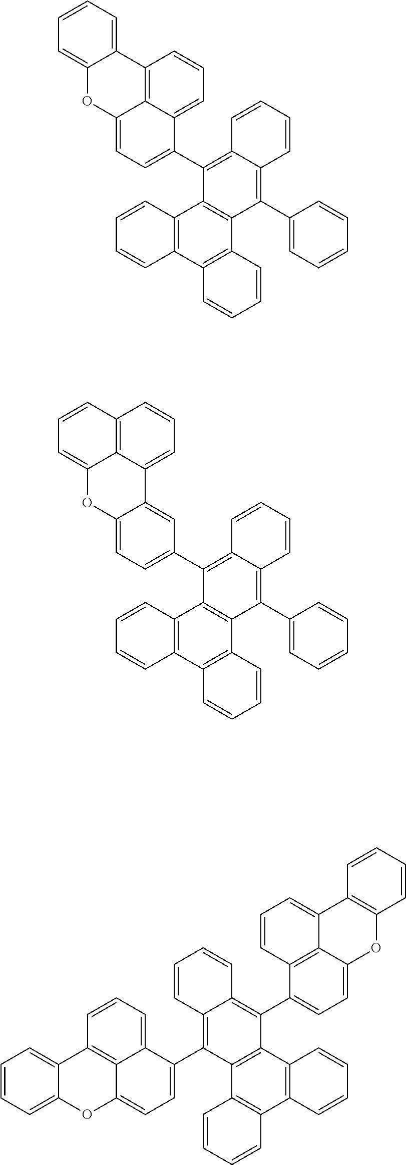 [SMILES: c1ccc(-c2c3ccccc3c(-c3ccc4c5c(cccc35)-c3ccccc3O4)c3c4ccccc4c4ccccc4c23)cc1 ; c1ccc(-c2c3ccccc3c(-c3ccc4c(c3)-c3cccc5cccc(c35)O4)c3c4ccccc4c4ccccc4c23)cc1 ; c1ccc2c(c1)Oc1ccc(-c3c4ccccc4c(-c4ccc5c6c(cccc46)-c4ccccc4O5)c4c5ccccc5c5ccccc5c34)c3cccc-2c13]

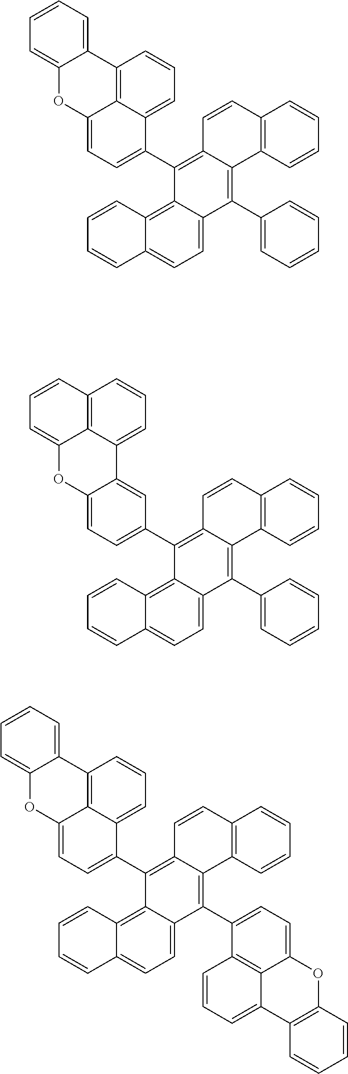 [SMILES: c1ccc(-c2c3ccc4ccccc4c3c(-c3ccc4c5c(cccc35)-c3ccccc3O4)c3ccc4ccccc4c23)cc1 ; c1ccc(-c2c3ccc4ccccc4c3c(-c3ccc4c(c3)-c3cccc5cccc(c35)O4)c3ccc4ccccc4c23)cc1 ; c1ccc2c(c1)Oc1ccc(-c3c4ccc5ccccc5c4c(-c4ccc5c6c(cccc46)-c4ccccc4O5)c4ccc5ccccc5c34)c3cccc-2c13]

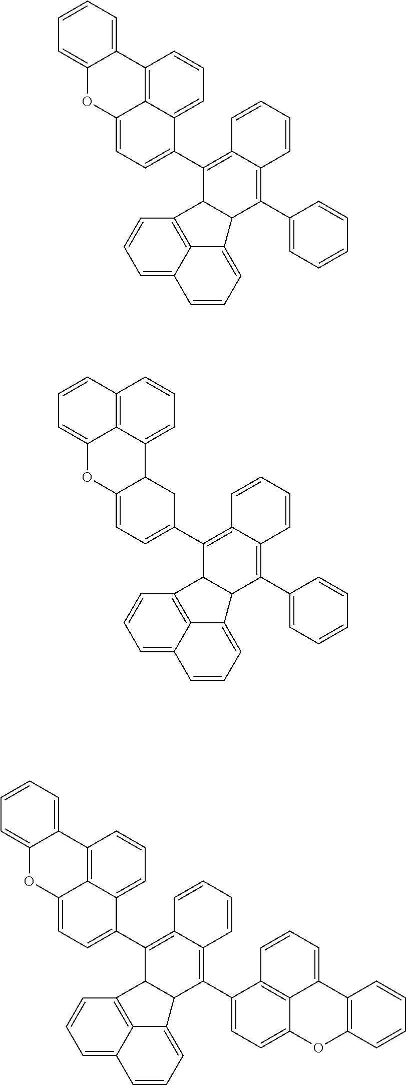 [SMILES: c1ccc(C2=c3ccccc3=C(c3ccc4c5c(cccc35)-c3ccccc3O4)C3c4cccc5cccc(c45)C23)cc1 ; C1=C(C2=c3ccccc3=C(c3ccccc3)C3c4cccc5cccc(c45)C23)CC2C(=C1)Oc1cccc3cccc2c13 ; c1ccc2c(c1)Oc1ccc(C3=c4ccccc4=C(c4ccc5c6c(cccc46)-c4ccccc4O5)C4c5cccc6cccc(c56)C34)c3cccc-2c13]

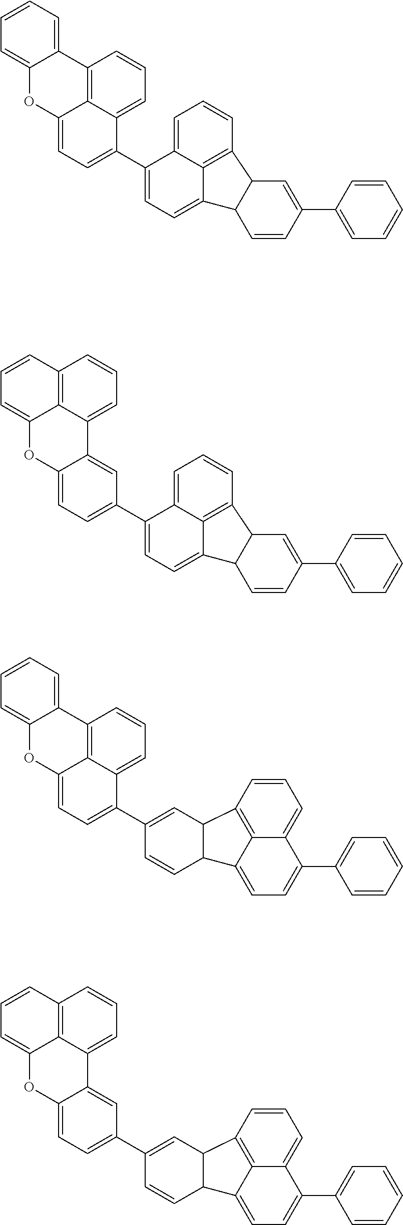 [SMILES: C1=CC2c3ccc(-c4ccc5c6c(cccc46)-c4ccccc4O5)c4cccc(c34)C2C=C1c1ccccc1 ; C1=CC2c3ccc(-c4ccc5c(c4)-c4cccc6cccc(c46)O5)c4cccc(c34)C2C=C1c1ccccc1 ; C1=CC2c3ccc(-c4ccccc4)c4cccc(c34)C2C=C1c1ccc2c3c(cccc13)-c1ccccc1O2 ; C1=CC2c3ccc(-c4ccccc4)c4cccc(c34)C2C=C1c1ccc2c(c1)-c1cccc3cccc(c13)O2]

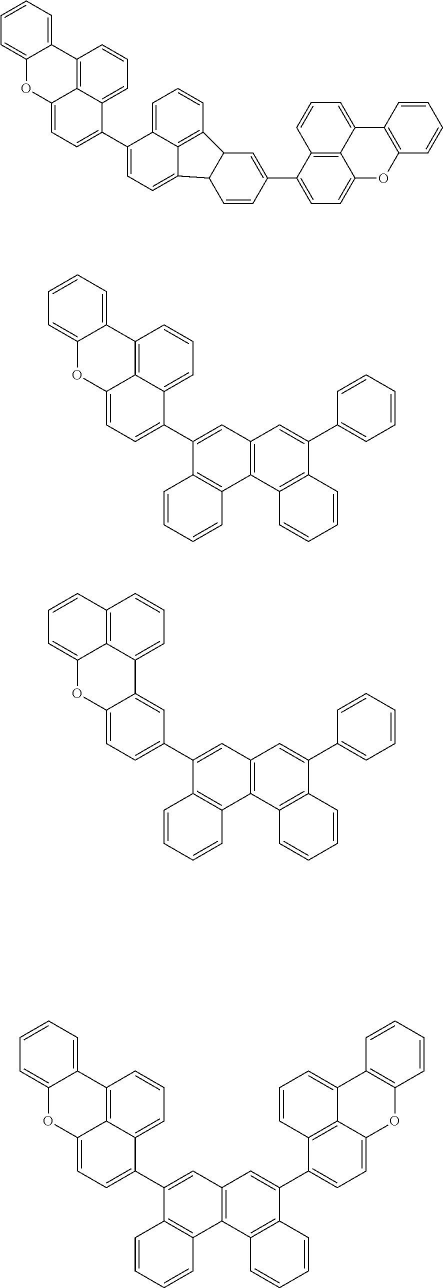 [SMILES: C1=CC2c3ccc(-c4ccc5c6c(cccc46)-c4ccccc4O5)c4cccc(c34)C2C=C1c1ccc2c3c(cccc13)-c1ccccc1O2 ; c1ccc(-c2cc3cc(-c4ccc5c6c(cccc46)-c4ccccc4O5)c4ccccc4c3c3ccccc23)cc1 ; c1ccc(-c2cc3cc(-c4ccc5c(c4)-c4cccc6cccc(c46)O5)c4ccccc4c3c3ccccc23)cc1 ; c1ccc2c(c1)Oc1ccc(-c3cc4cc(-c5ccc6c7c(cccc57)-c5ccccc5O6)c5ccccc5c4c4ccccc34)c3cccc-2c13]

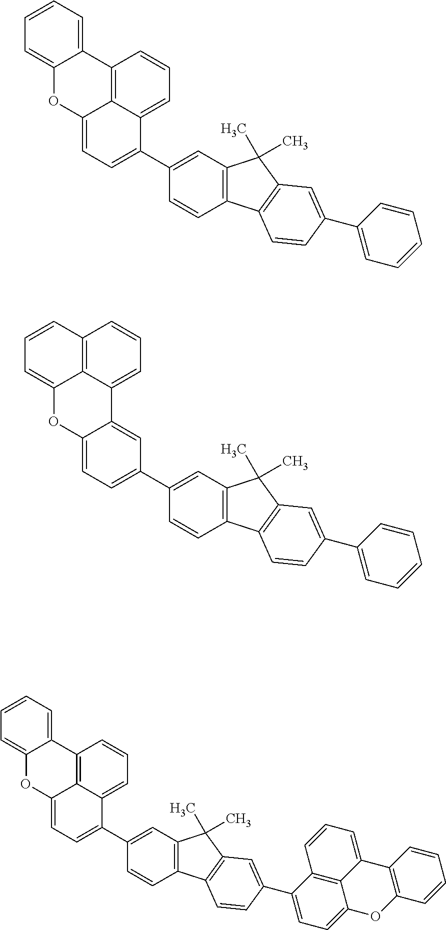 [SMILES: CC1(C)c2cc(-c3ccccc3)ccc2-c2ccc(-c3ccc4c5c(cccc35)-c3ccccc3O4)cc21 ; CC1(C)c2cc(-c3ccccc3)ccc2-c2ccc(-c3ccc4c(c3)-c3cccc5cccc(c35)O4)cc21 ; CC1(C)c2cc(-c3ccc4c5c(cccc35)-c3ccccc3O4)ccc2-c2ccc(-c3ccc4c5c(cccc35)-c3ccccc3O4)cc21]

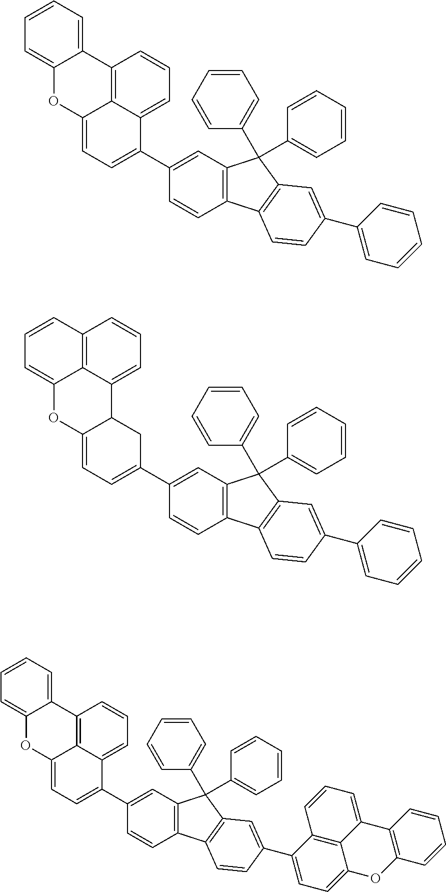 [SMILES: c1ccc(-c2ccc3c(c2)C(c2ccccc2)(c2ccccc2)c2cc(-c4ccc5c6c(cccc46)-c4ccccc4O5)ccc2-3)cc1 ; C1=C(c2ccc3c(c2)C(c2ccccc2)(c2ccccc2)c2cc(-c4ccccc4)ccc2-3)CC2C(=C1)Oc1cccc3cccc2c13 ; c1ccc(C2(c3ccccc3)c3cc(-c4ccc5c6c(cccc46)-c4ccccc4O5)ccc3-c3ccc(-c4ccc5c6c(cccc46)-c4ccccc4O5)cc32)cc1]

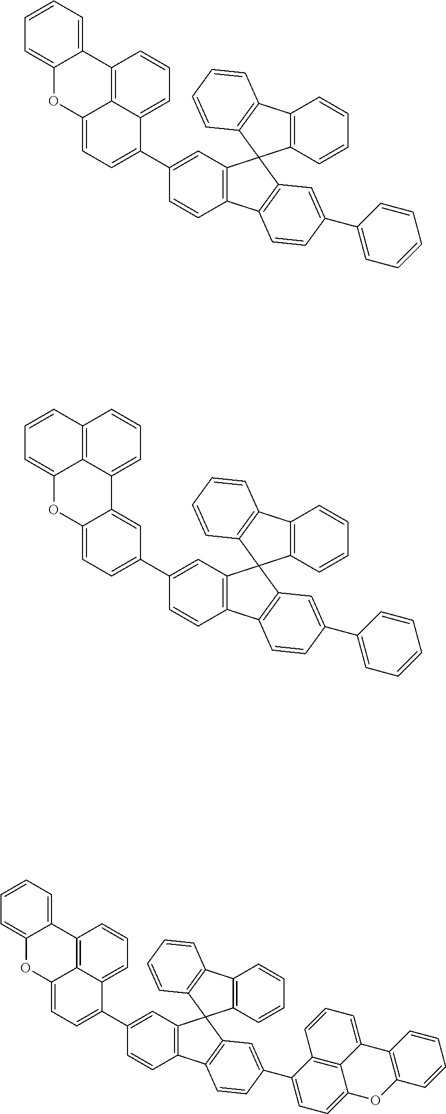 [SMILES: c1ccc(-c2ccc3c(c2)C2(c4ccccc4-c4ccccc42)c2cc(-c4ccc5c6c(cccc46)-c4ccccc4O5)ccc2-3)cc1 ; c1ccc(-c2ccc3c(c2)C2(c4ccccc4-c4ccccc42)c2cc(-c4ccc5c(c4)-c4cccc6cccc(c46)O5)ccc2-3)cc1 ; c1ccc2c(c1)Oc1ccc(-c3ccc4c(c3)C3(c5ccccc5-c5ccccc53)c3cc(-c5ccc6c7c(cccc57)-c5ccccc5O6)ccc3-4)c3cccc-2c13]

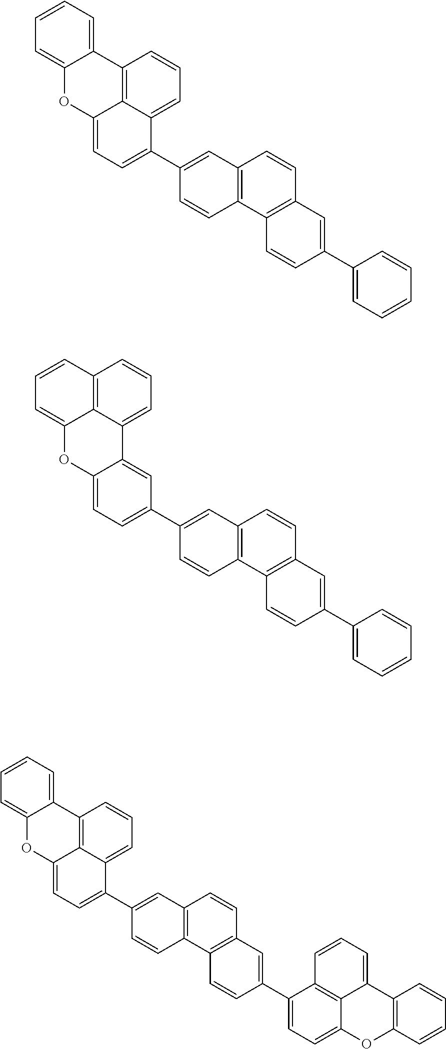 [SMILES: c1ccc(-c2ccc3c(ccc4cc(-c5ccc6c7c(cccc57)-c5ccccc5O6)ccc43)c2)cc1 ; c1ccc(-c2ccc3c(ccc4cc(-c5ccc6c(c5)-c5cccc7cccc(c57)O6)ccc43)c2)cc1 ; c1ccc2c(c1)Oc1ccc(-c3ccc4c(ccc5cc(-c6ccc7c8c(cccc68)-c6ccccc6O7)ccc54)c3)c3cccc-2c13]

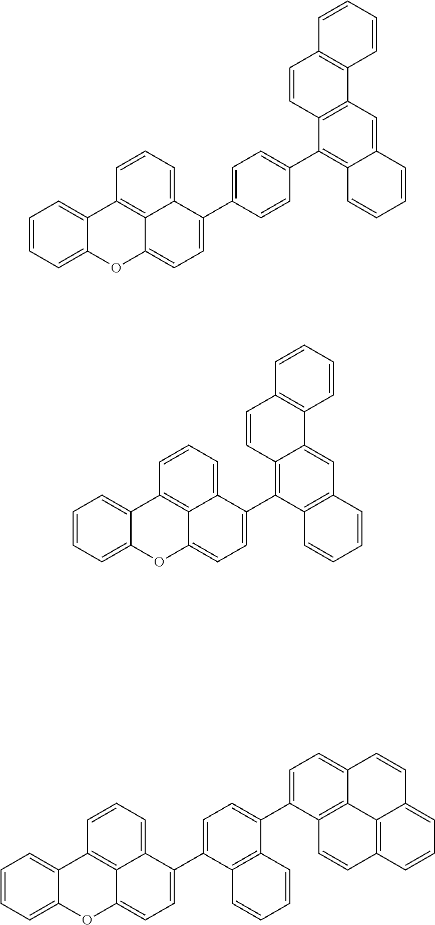 [SMILES: c1ccc2c(c1)Oc1ccc(-c3ccc(-c4c5ccccc5cc5c4ccc4ccccc45)cc3)c3cccc-2c13 ; c1ccc2c(c1)Oc1ccc(-c3c4ccccc4cc4c3ccc3ccccc34)c3cccc-2c13 ; c1ccc2c(c1)Oc1ccc(-c3ccc(-c4ccc5ccc6cccc7ccc4c5c67)c4ccccc34)c3cccc-2c13]

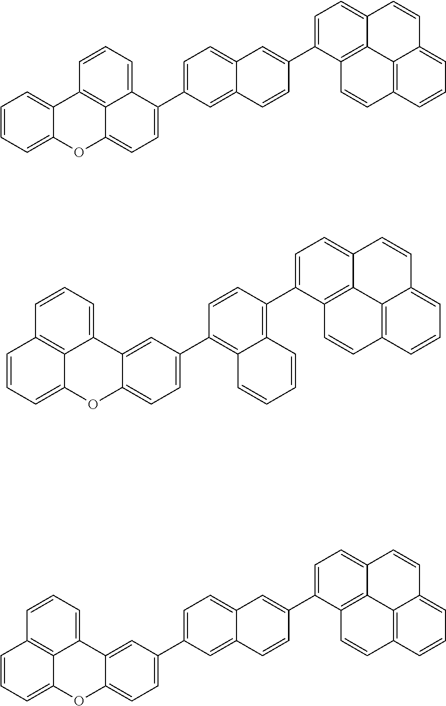 [SMILES: c1ccc2c(c1)Oc1ccc(-c3ccc4cc(-c5ccc6ccc7cccc8ccc5c6c78)ccc4c3)c3cccc-2c13 ; c1cc2c3c(cccc3c1)-c1cc(-c3ccc(-c4ccc5ccc6cccc7ccc4c5c67)c4ccccc34)ccc1O2 ; c1cc2c3c(cccc3c1)-c1cc(-c3ccc4cc(-c5ccc6ccc7cccc8ccc5c6c78)ccc4c3)ccc1O2]

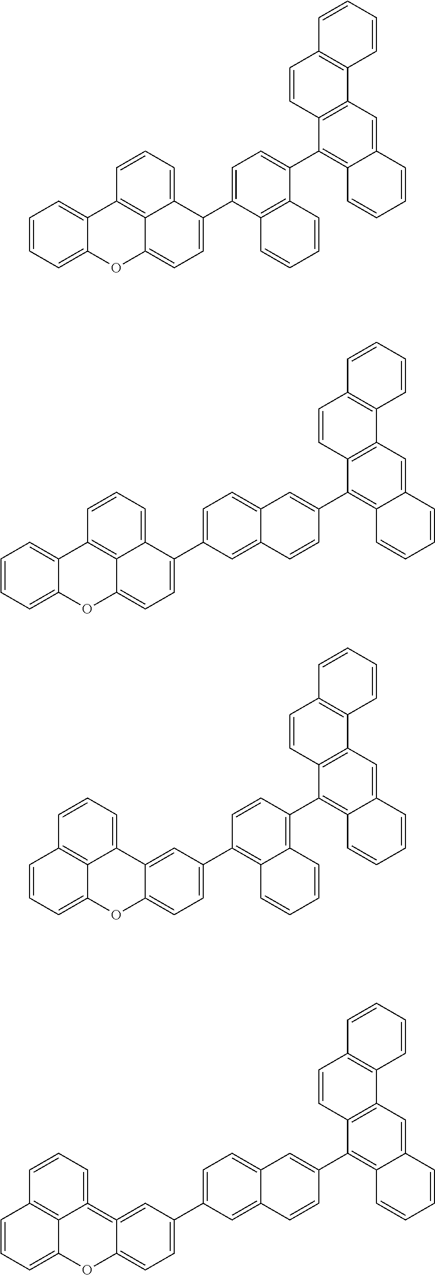

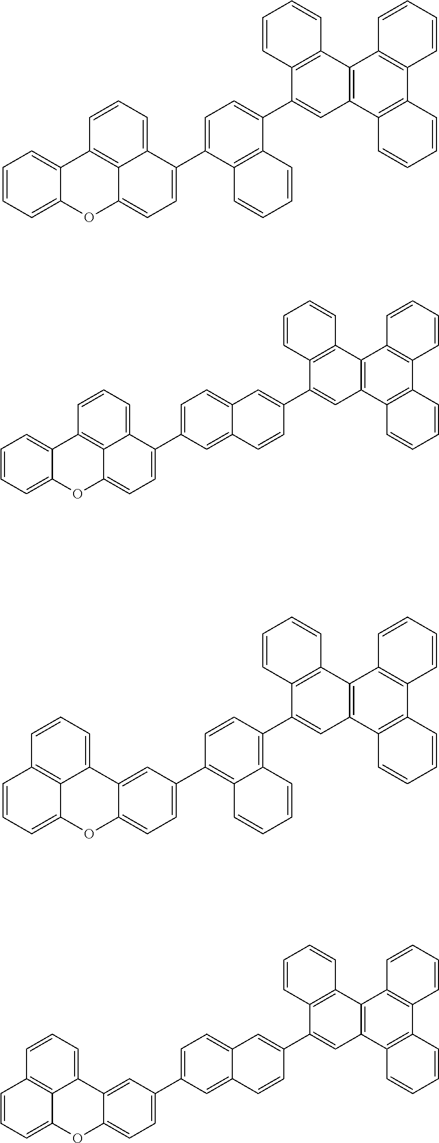 [SMILES: c1ccc2c(c1)Oc1ccc(-c3ccc(-c4cc5c6ccccc6c6ccccc6c5c5ccccc45)c4ccccc34)c3cccc-2c13 ; c1ccc2c(c1)Oc1ccc(-c3ccc4cc(-c5cc6c7ccccc7c7ccccc7c6c6ccccc56)ccc4c3)c3cccc-2c13 ; c1cc2c3c(cccc3c1)-c1cc(-c3ccc(-c4cc5c6ccccc6c6ccccc6c5c5ccccc45)c4ccccc34)ccc1O2 ; c1cc2c3c(cccc3c1)-c1cc(-c3ccc4cc(-c5cc6c7ccccc7c7ccccc7c6c6ccccc56)ccc4c3)ccc1O2]

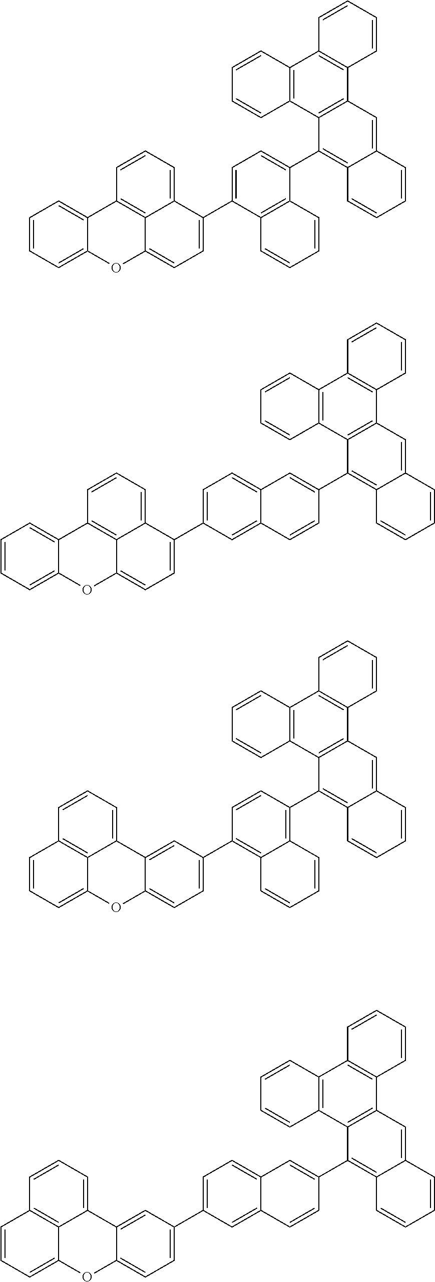 [SMILES: c1ccc2c(c1)Oc1ccc(-c3ccc(-c4c5ccccc5cc5c6ccccc6c6ccccc6c45)c4ccccc34)c3cccc-2c13 ; c1ccc2c(c1)Oc1ccc(-c3ccc4cc(-c5c6ccccc6cc6c7ccccc7c7ccccc7c56)ccc4c3)c3cccc-2c13 ; c1ccc2c(-c3ccc(-c4ccc5c(c4)-c4cccc6cccc(c46)O5)c4ccccc34)c3c4ccccc4c4ccccc4c3cc2c1 ; c1ccc2c(-c3ccc4cc(-c5ccc6c(c5)-c5cccc7cccc(c57)O6)ccc4c3)c3c4ccccc4c4ccccc4c3cc2c1]

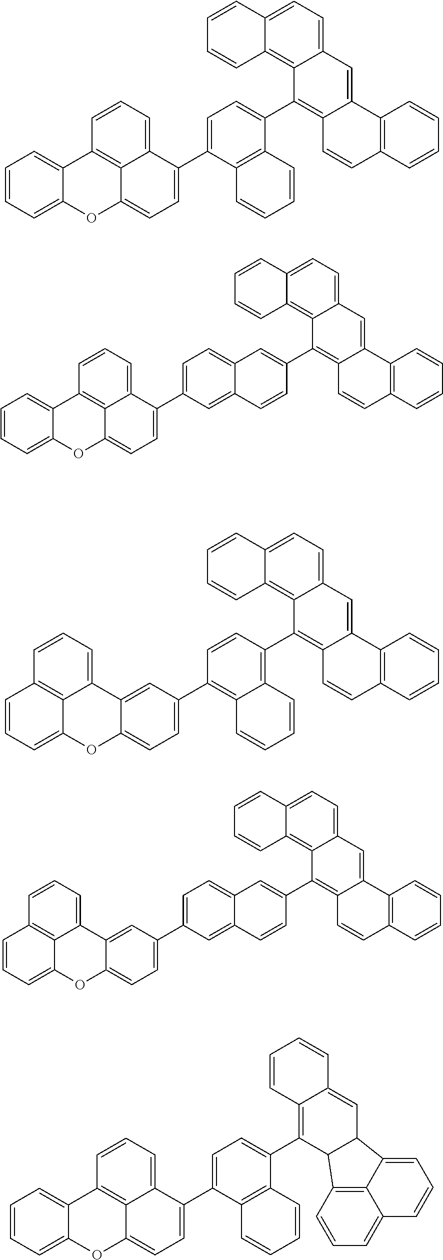

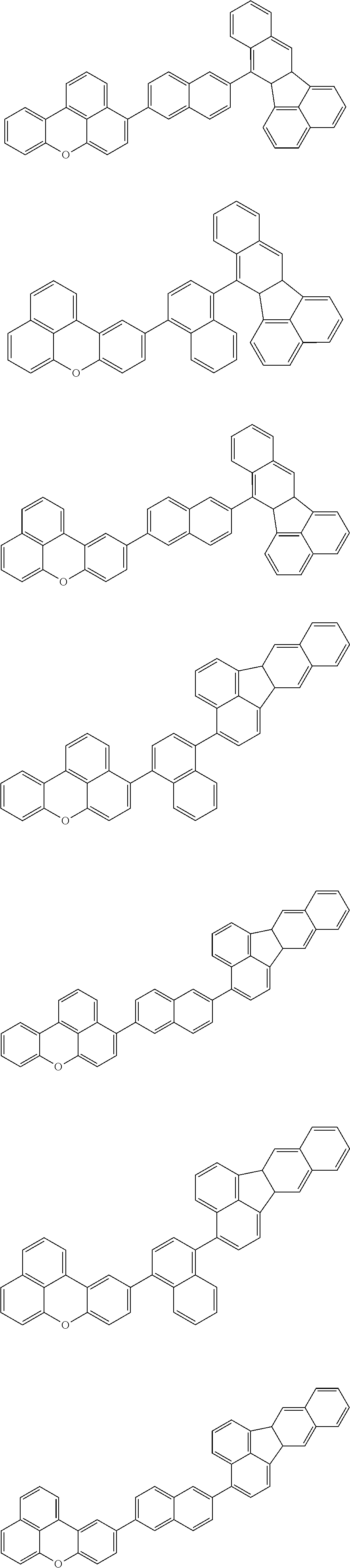

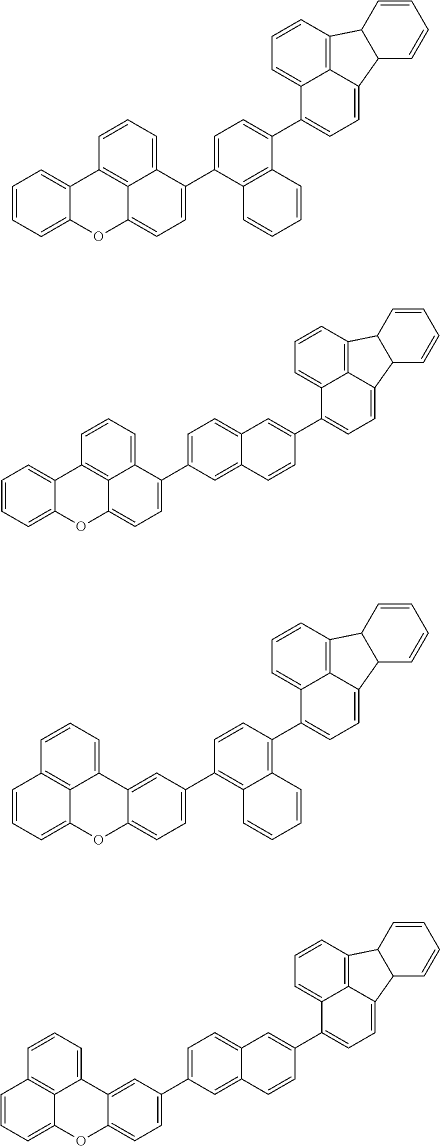 [SMILES: C1=CC2c3cccc4c(-c5ccc(-c6ccc7c8c(cccc68)-c6ccccc6O7)c6ccccc56)ccc(c34)C2C=C1 ; C1=CC2c3cccc4c(-c5ccc6cc(-c7ccc8c9c(cccc79)-c7ccccc7O8)ccc6c5)ccc(c34)C2C=C1 ; C1=CC2c3cccc4c(-c5ccc(-c6ccc7c(c6)-c6cccc8cccc(c68)O7)c6ccccc56)ccc(c34)C2C=C1 ; C1=CC2c3cccc4c(-c5ccc6cc(-c7ccc8c(c7)-c7cccc9cccc(c79)O8)ccc6c5)ccc(c34)C2C=C1]

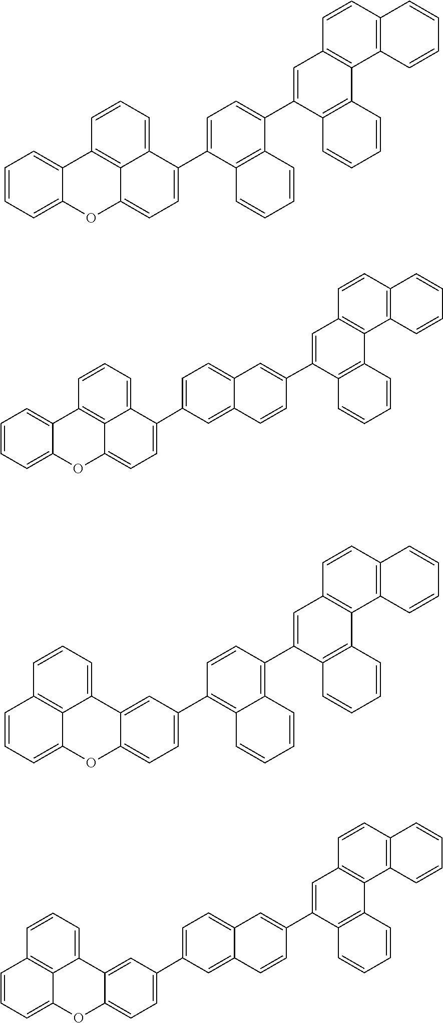 [SMILES: c1ccc2c(c1)Oc1ccc(-c3ccc(-c4cc5ccc6ccccc6c5c5ccccc45)c4ccccc34)c3cccc-2c13 ; c1ccc2c(c1)Oc1ccc(-c3ccc4cc(-c5cc6ccc7ccccc7c6c6ccccc56)ccc4c3)c3cccc-2c13 ; c1cc2c3c(cccc3c1)-c1cc(-c3ccc(-c4cc5ccc6ccccc6c5c5ccccc45)c4ccccc34)ccc1O2 ; c1cc2c3c(cccc3c1)-c1cc(-c3ccc4cc(-c5cc6ccc7ccccc7c6c6ccccc56)ccc4c3)ccc1O2]

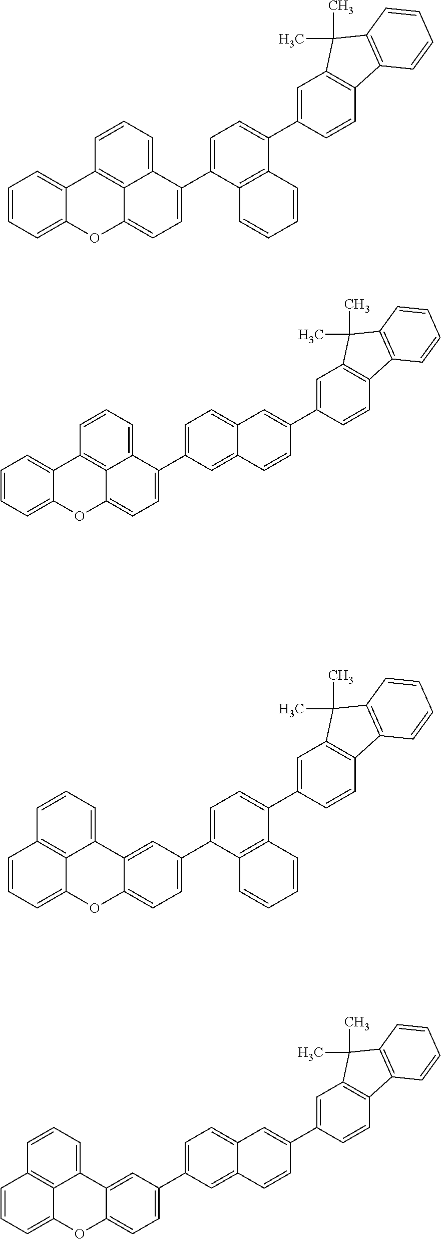 [SMILES: CC1(C)c2ccccc2-c2ccc(-c3ccc(-c4ccc5c6c(cccc46)-c4ccccc4O5)c4ccccc34)cc21 ; CC1(C)c2ccccc2-c2ccc(-c3ccc4cc(-c5ccc6c7c(cccc57)-c5ccccc5O6)ccc4c3)cc21 ; CC1(C)c2ccccc2-c2ccc(-c3ccc(-c4ccc5c(c4)-c4cccc6cccc(c46)O5)c4ccccc34)cc21 ; CC1(C)c2ccccc2-c2ccc(-c3ccc4cc(-c5ccc6c(c5)-c5cccc7cccc(c57)O6)ccc4c3)cc21]

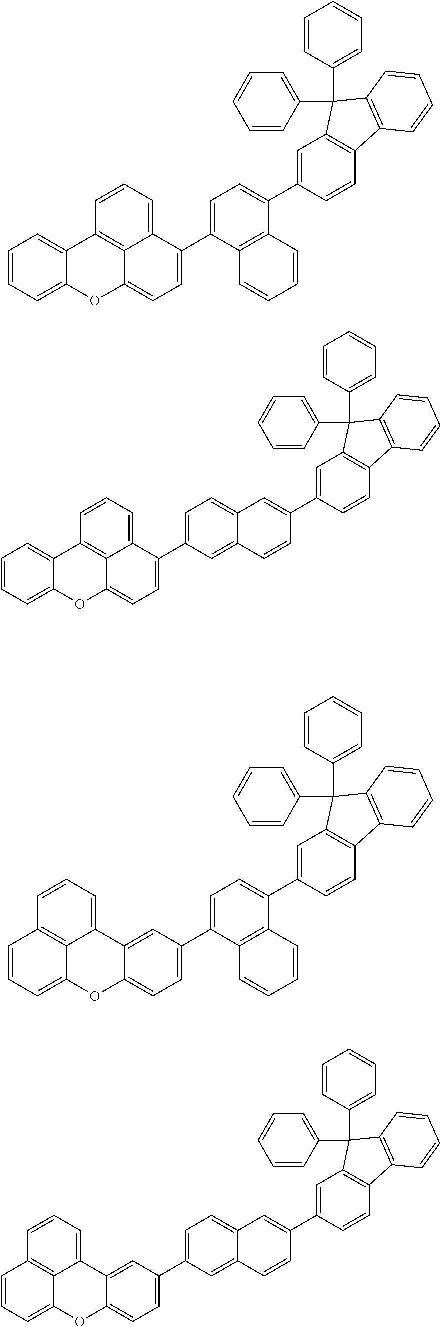 [SMILES: c1ccc(C2(c3ccccc3)c3ccccc3-c3ccc(-c4ccc(-c5ccc6c7c(cccc57)-c5ccccc5O6)c5ccccc45)cc32)cc1 ; c1ccc(C2(c3ccccc3)c3ccccc3-c3ccc(-c4ccc5cc(-c6ccc7c8c(cccc68)-c6ccccc6O7)ccc5c4)cc32)cc1 ; c1ccc(C2(c3ccccc3)c3ccccc3-c3ccc(-c4ccc(-c5ccc6c(c5)-c5cccc7cccc(c57)O6)c5ccccc45)cc32)cc1 ; c1ccc(C2(c3ccccc3)c3ccccc3-c3ccc(-c4ccc5cc(-c6ccc7c(c6)-c6cccc8cccc(c68)O7)ccc5c4)cc32)cc1]

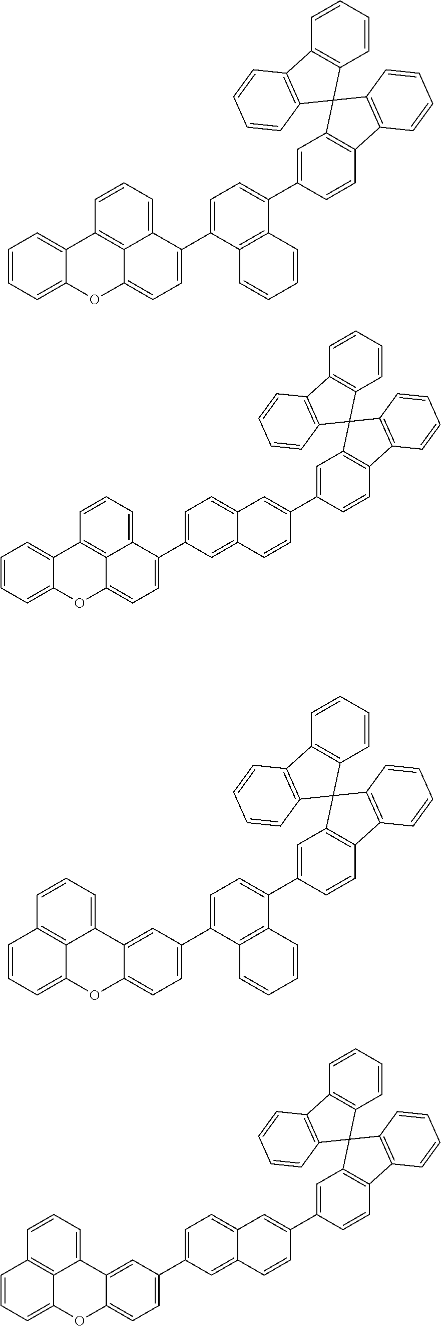 [SMILES: c1ccc2c(c1)Oc1ccc(-c3ccc(-c4ccc5c(c4)C4(c6ccccc6-c6ccccc64)c4ccccc4-5)c4ccccc34)c3cccc-2c13 ; c1ccc2c(c1)Oc1ccc(-c3ccc4cc(-c5ccc6c(c5)C5(c7ccccc7-c7ccccc75)c5ccccc5-6)ccc4c3)c3cccc-2c13 ; c1ccc2c(c1)-c1ccccc1C21c2ccccc2-c2ccc(-c3ccc(-c4ccc5c(c4)-c4cccc6cccc(c46)O5)c4ccccc34)cc21 ; c1ccc2c(c1)-c1ccccc1C21c2ccccc2-c2ccc(-c3ccc4cc(-c5ccc6c(c5)-c5cccc7cccc(c57)O6)ccc4c3)cc21]

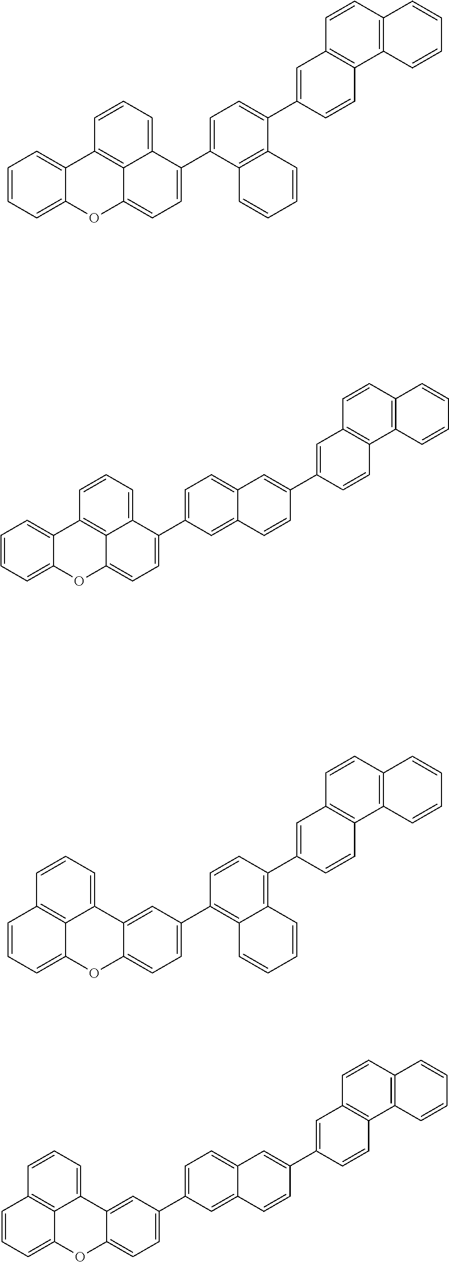 [SMILES: c1ccc2c(c1)Oc1ccc(-c3ccc(-c4ccc5c(ccc6ccccc65)c4)c4ccccc34)c3cccc-2c13 ; c1ccc2c(c1)Oc1ccc(-c3ccc4cc(-c5ccc6c(ccc7ccccc76)c5)ccc4c3)c3cccc-2c13 ; c1cc2c3c(cccc3c1)-c1cc(-c3ccc(-c4ccc5c(ccc6ccccc65)c4)c4ccccc34)ccc1O2 ; c1cc2c3c(cccc3c1)-c1cc(-c3ccc4cc(-c5ccc6c(ccc7ccccc76)c5)ccc4c3)ccc1O2]

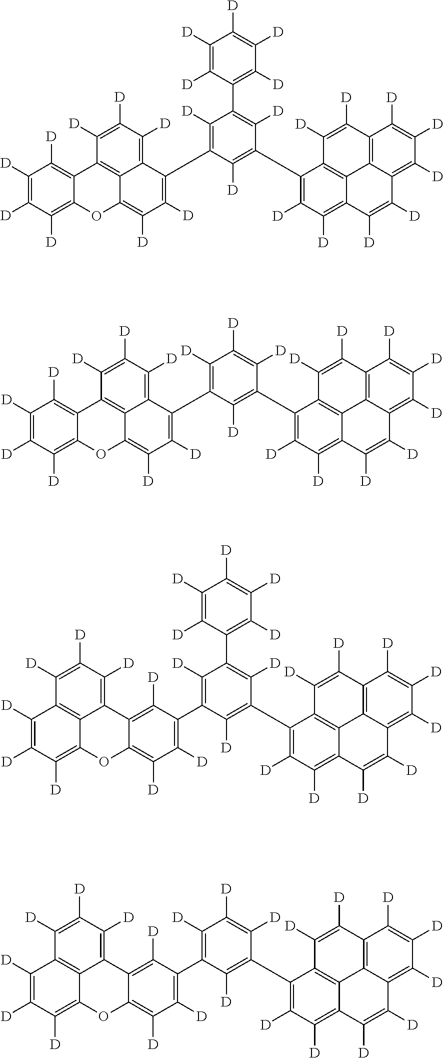

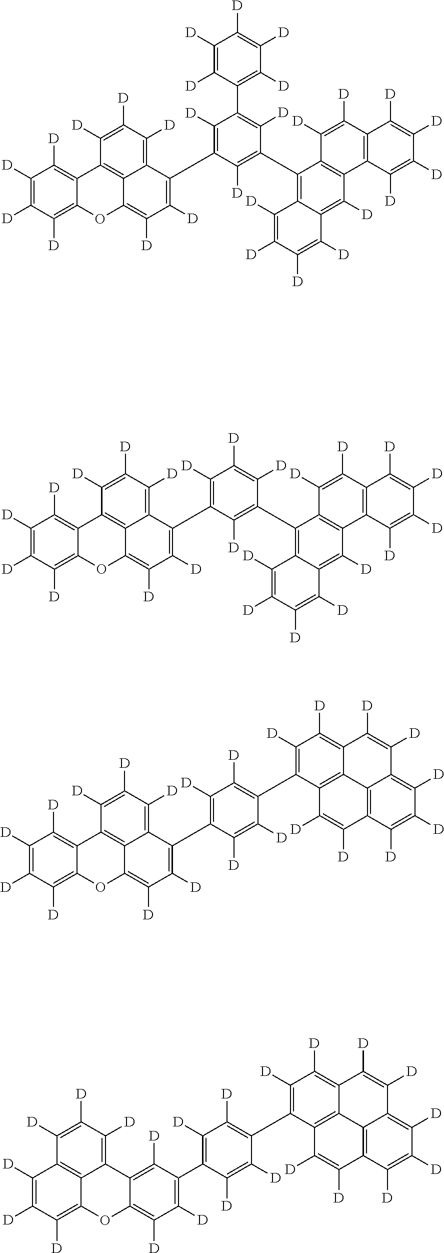

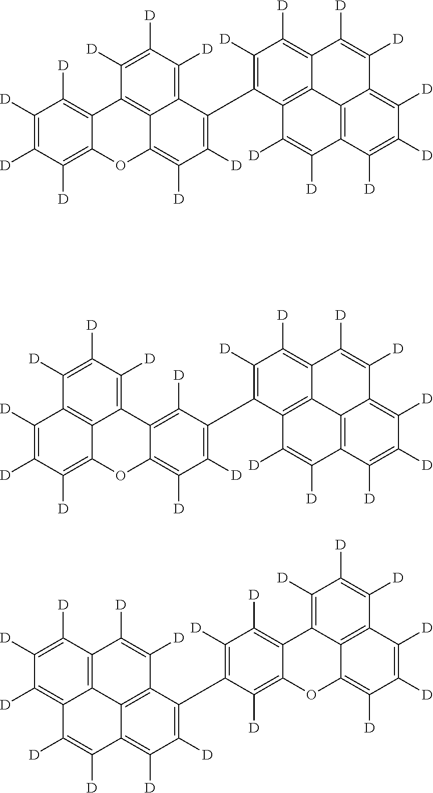 [SMILES: [2H]c1c([2H])c([2H])c2c(c1[2H])Oc1c([2H])c([2H])c(-c3c([2H])c([2H])c4c([2H])c([2H])c5c([2H])c([2H])c([2H])c6c([2H])c([2H])c3c4c56)c3c([2H])c([2H])c([2H])c-2c13 ; [2H]c1c([2H])c(-c2c([2H])c([2H])c3c([2H])c([2H])c4c([2H])c([2H])c([2H])c5c([2H])c([2H])c2c3c45)c([2H])c2c1Oc1c([2H])c([2H])c([2H])c3c([2H])c([2H])c([2H])c-2c13 ; [2H]c1c([2H])c2c(c([2H])c1-c1c([2H])c([2H])c3c([2H])c([2H])c4c([2H])c([2H])c([2H])c5c([2H])c([2H])c1c3c45)Oc1c([2H])c([2H])c([2H])c3c([2H])c([2H])c([2H])c-2c13]

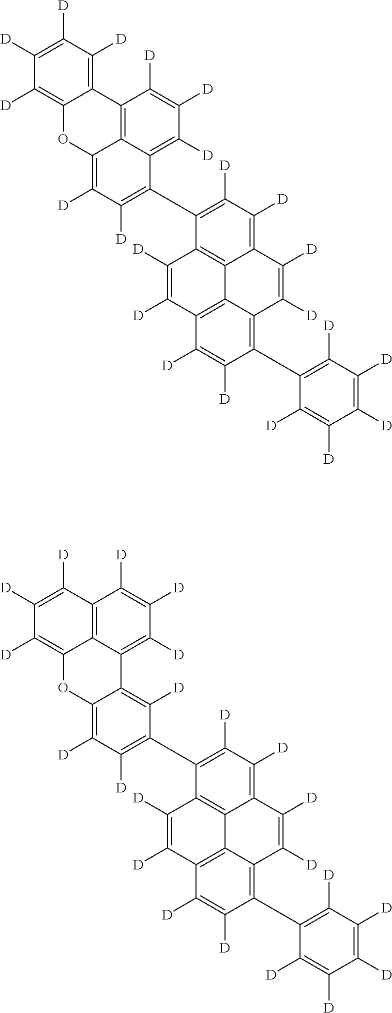 [SMILES: [2H]c1c([2H])c([2H])c(-c2c([2H])c([2H])c3c([2H])c([2H])c4c(-c5c([2H])c([2H])c6c7c(c([2H])c([2H])c([2H])c57)-c5c([2H])c([2H])c([2H])c([2H])c5O6)c([2H])c([2H])c5c([2H])c([2H])c2c3c54)c([2H])c1[2H] ; [2H]c1c([2H])c([2H])c(-c2c([2H])c([2H])c3c([2H])c([2H])c4c(-c5c([2H])c([2H])c6c(c5[2H])-c5c([2H])c([2H])c([2H])c7c([2H])c([2H])c([2H])c(c57)O6)c([2H])c([2H])c5c([2H])c([2H])c2c3c54)c([2H])c1[2H]]

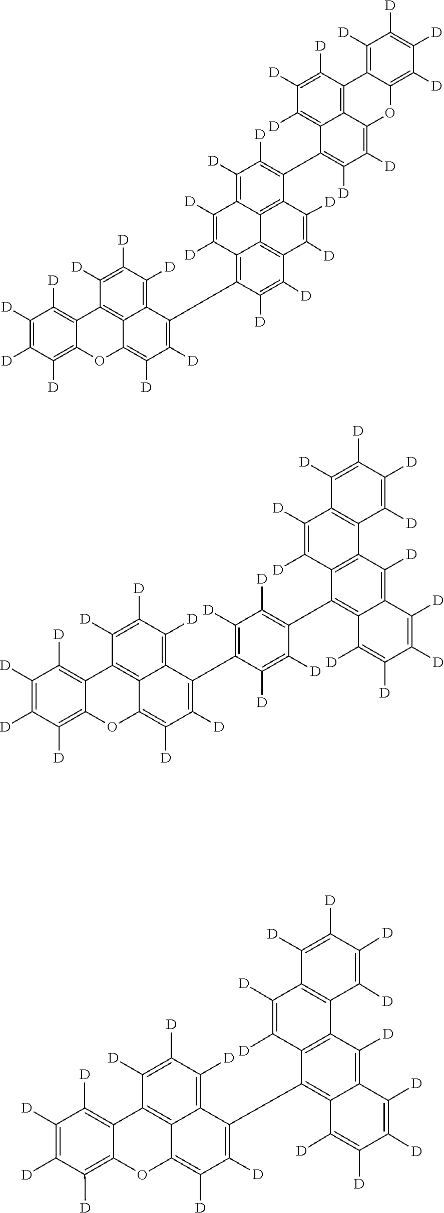

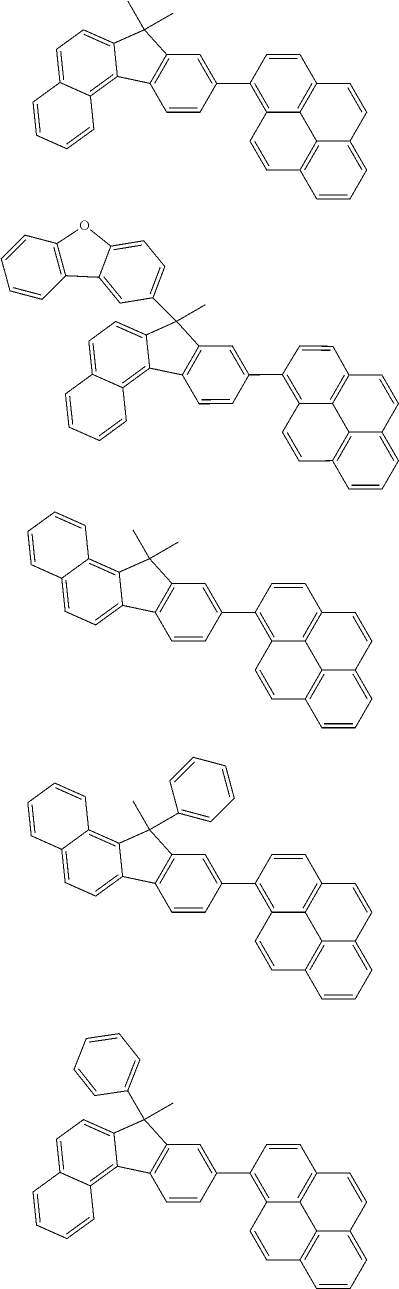

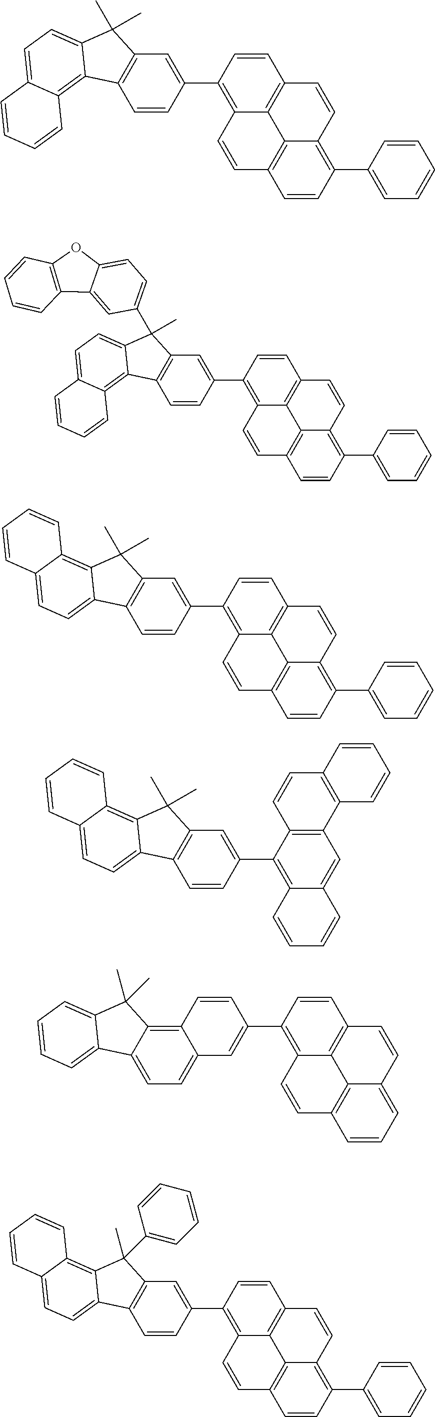

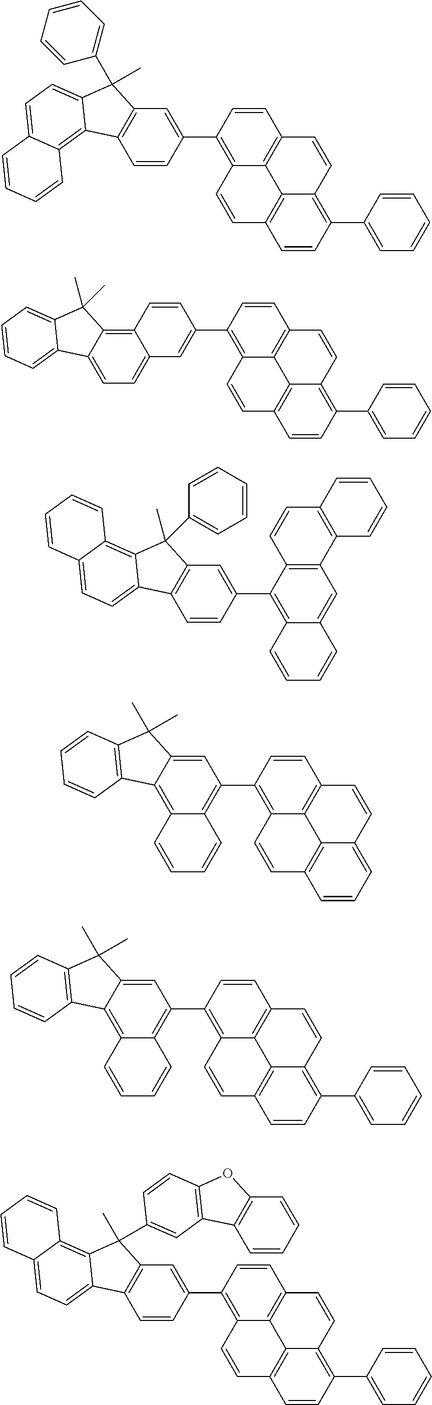

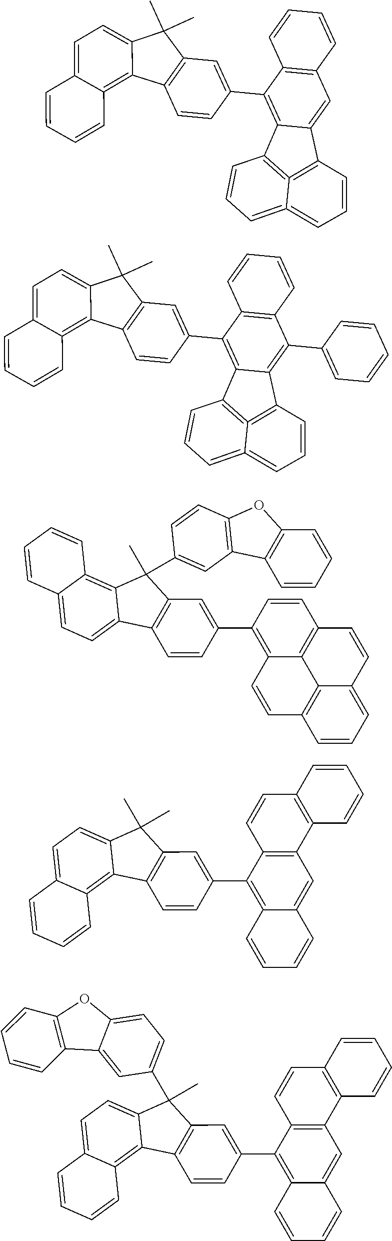

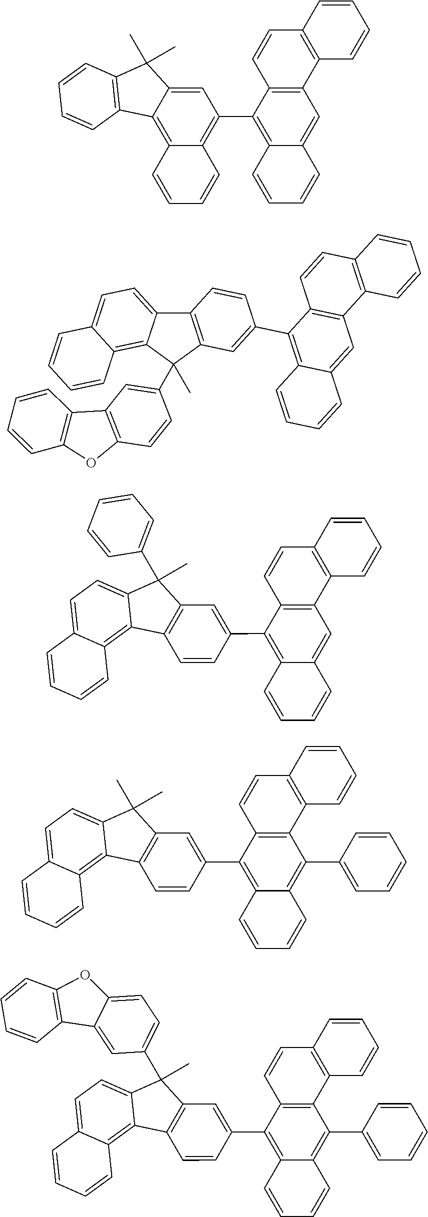

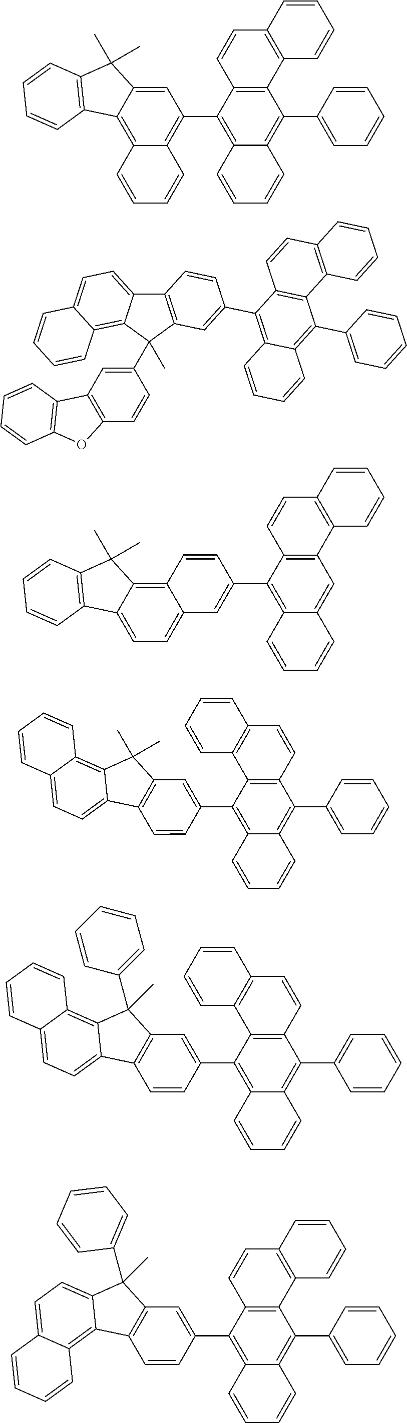

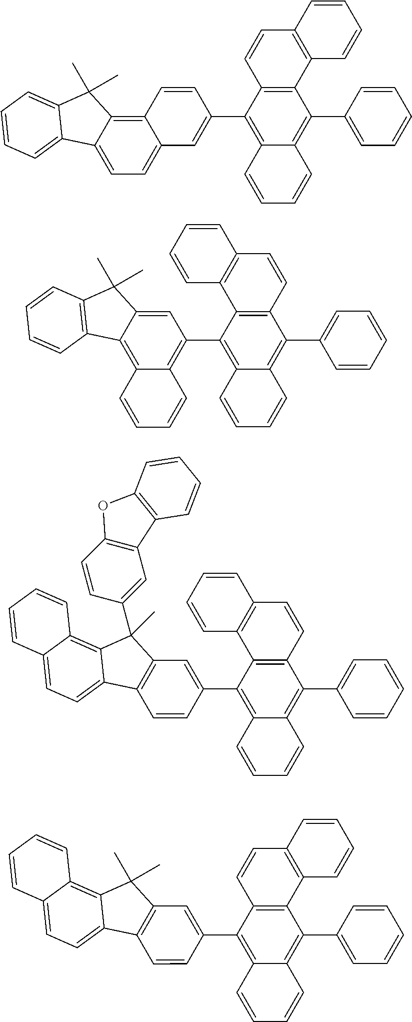

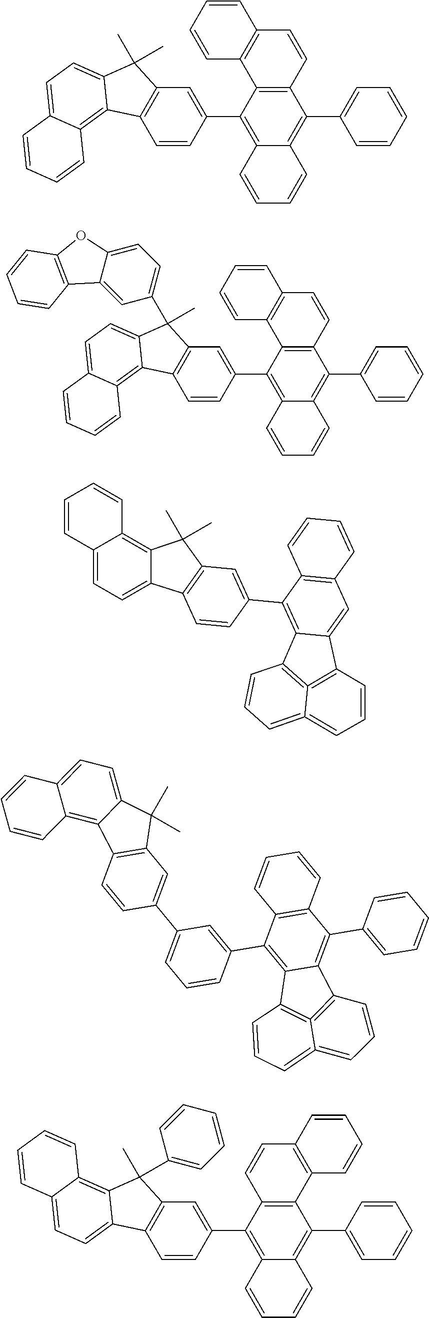

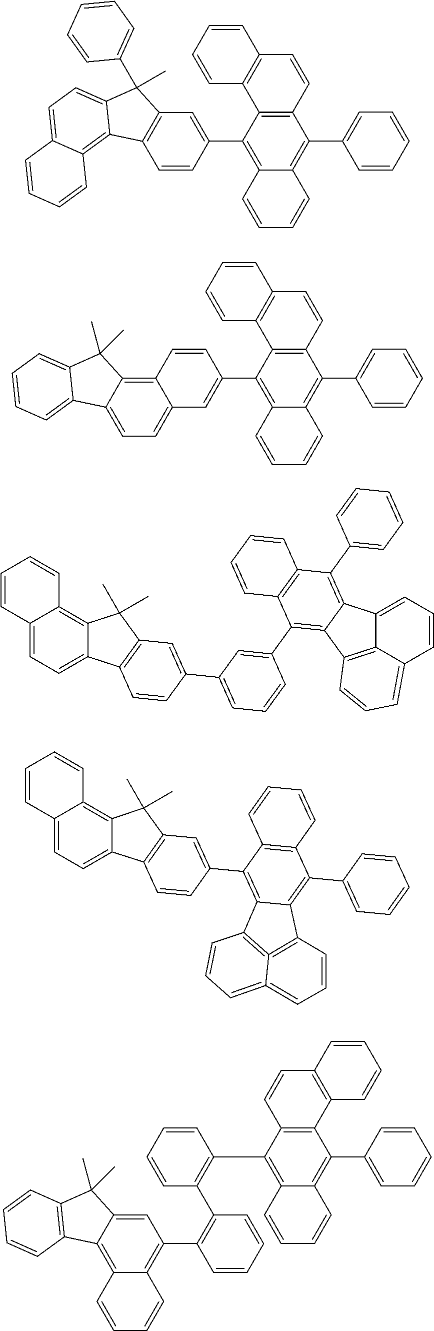

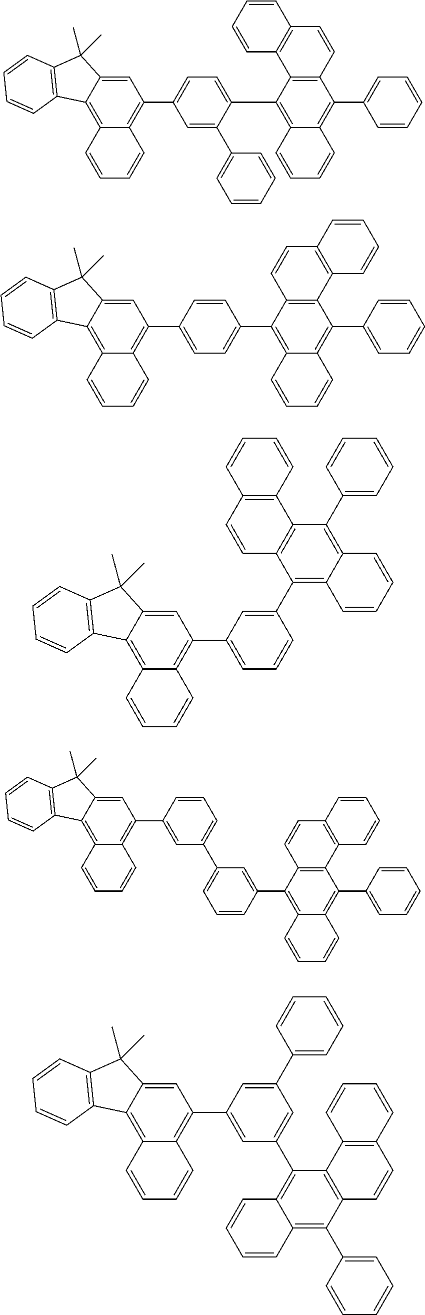

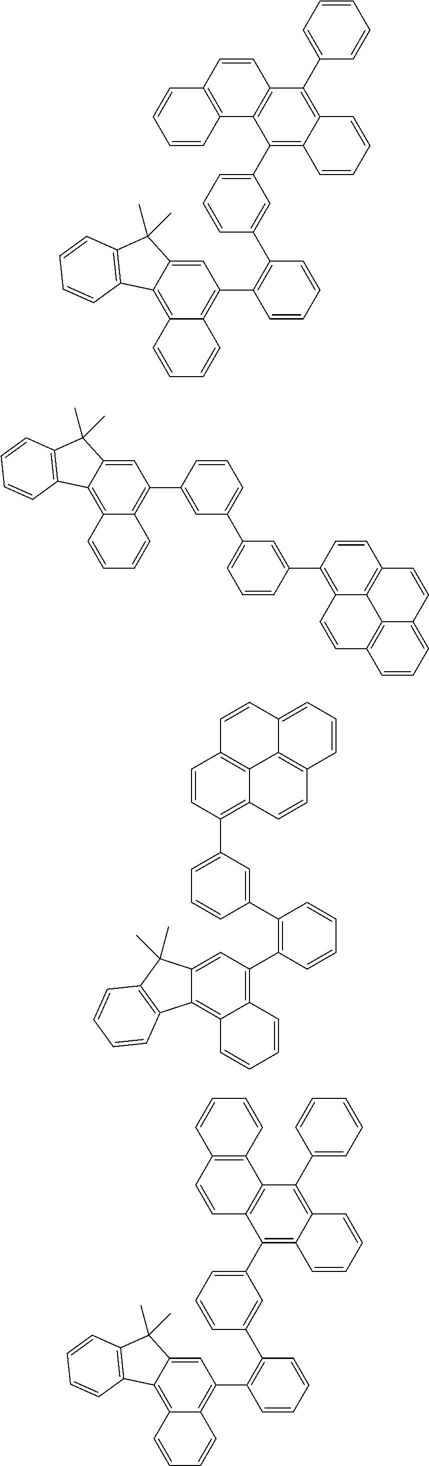

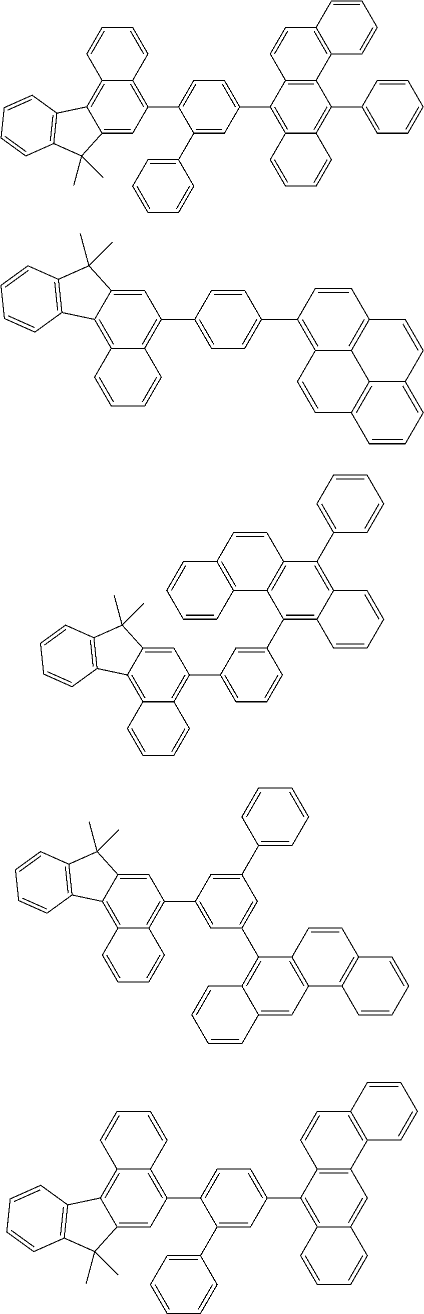

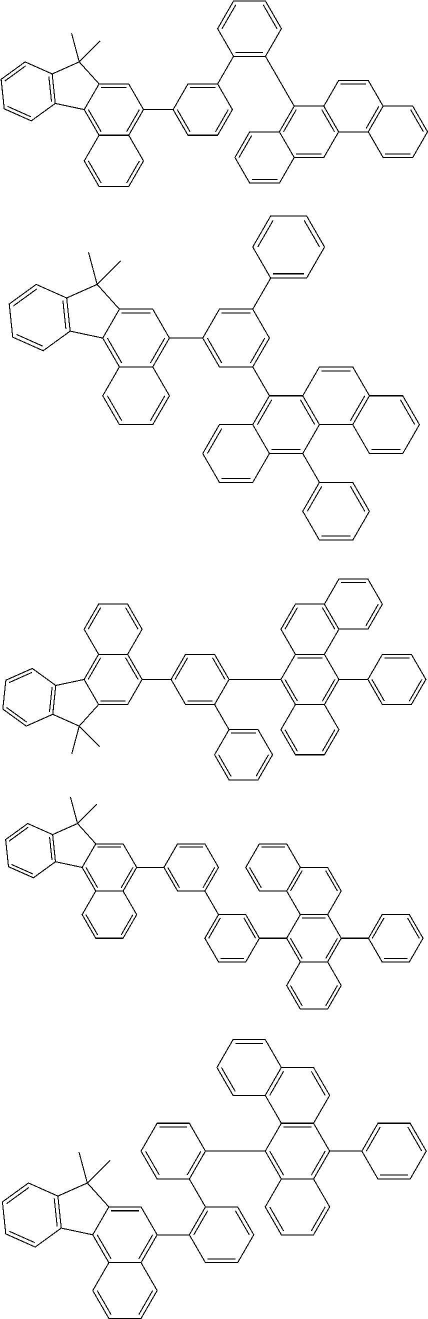

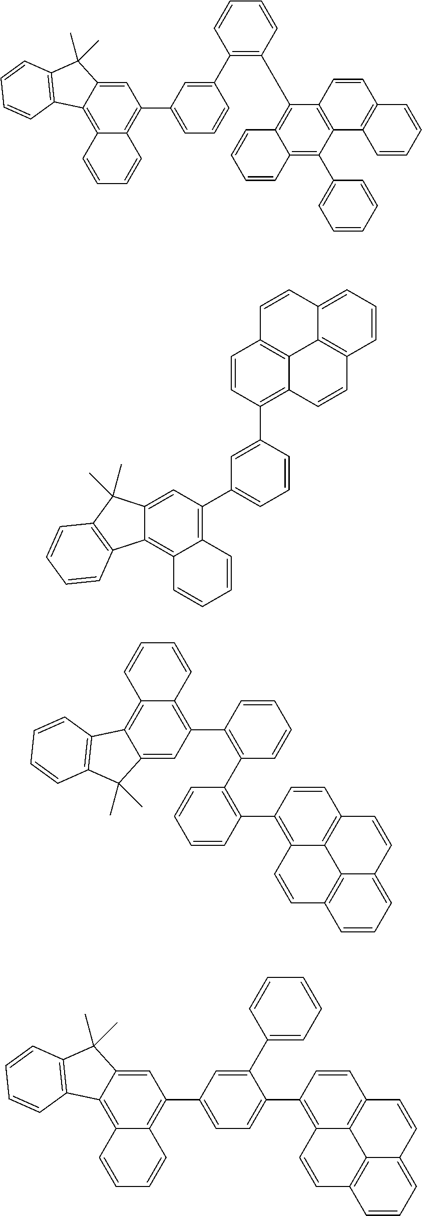

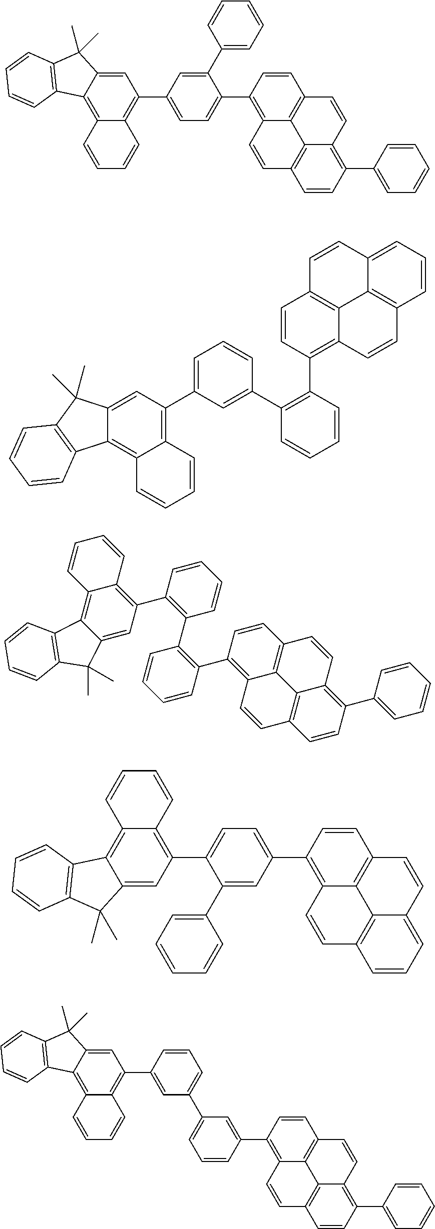

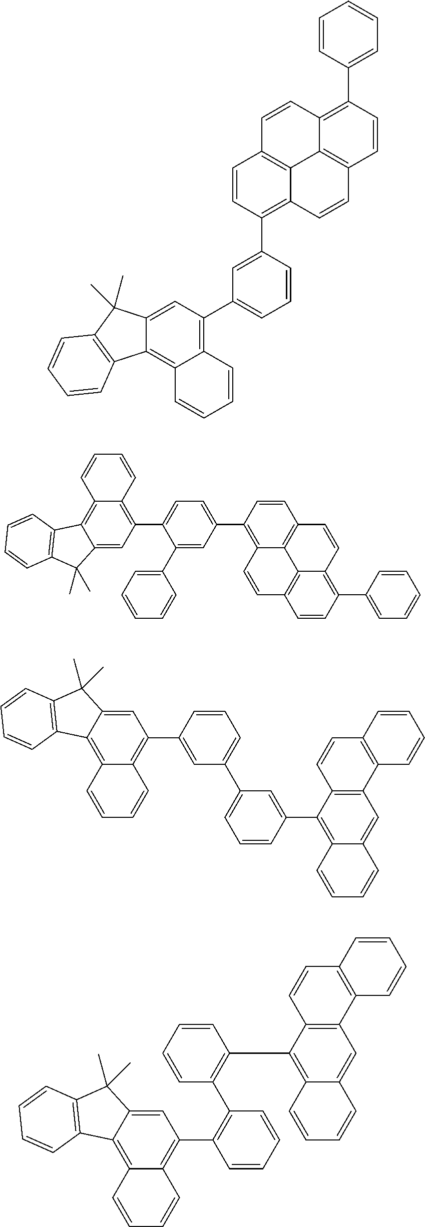

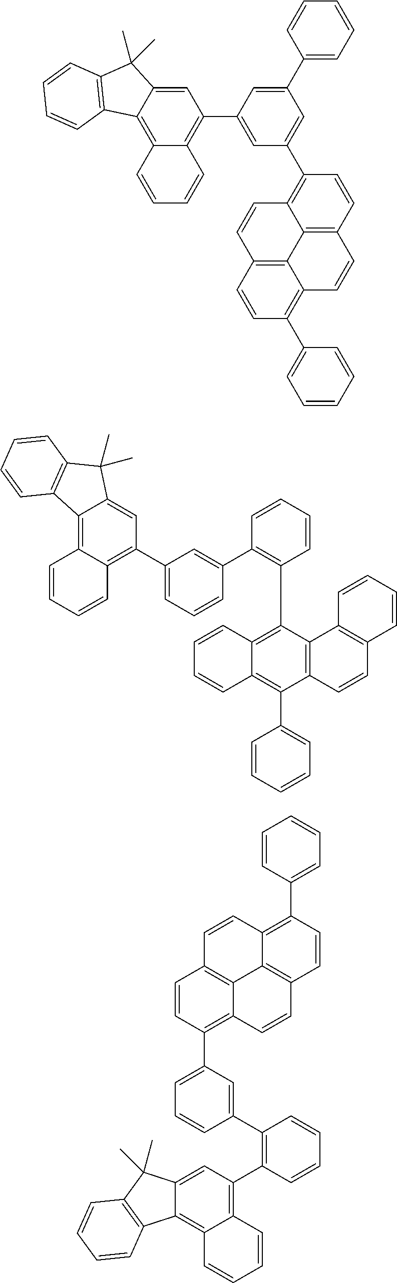

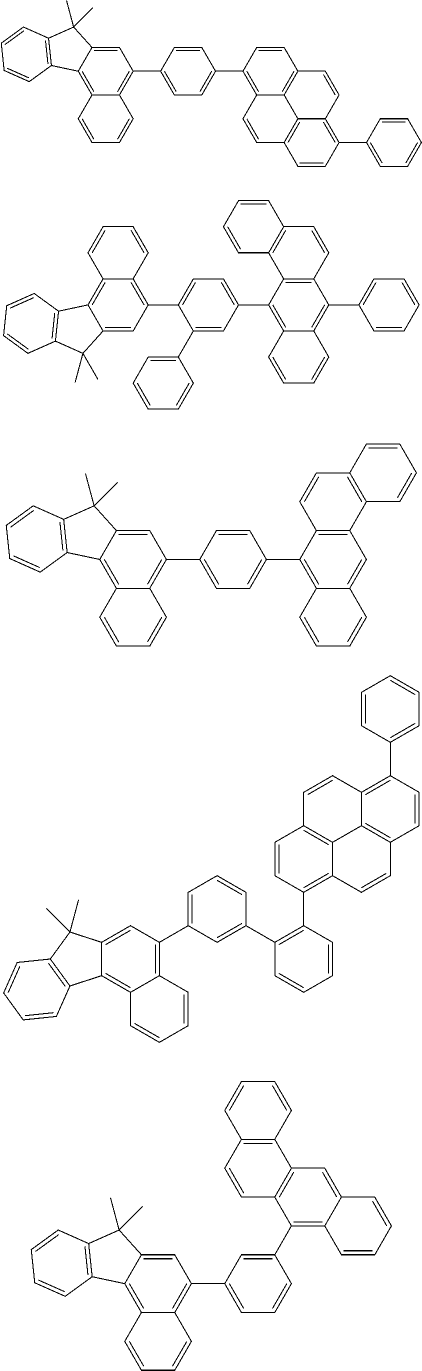

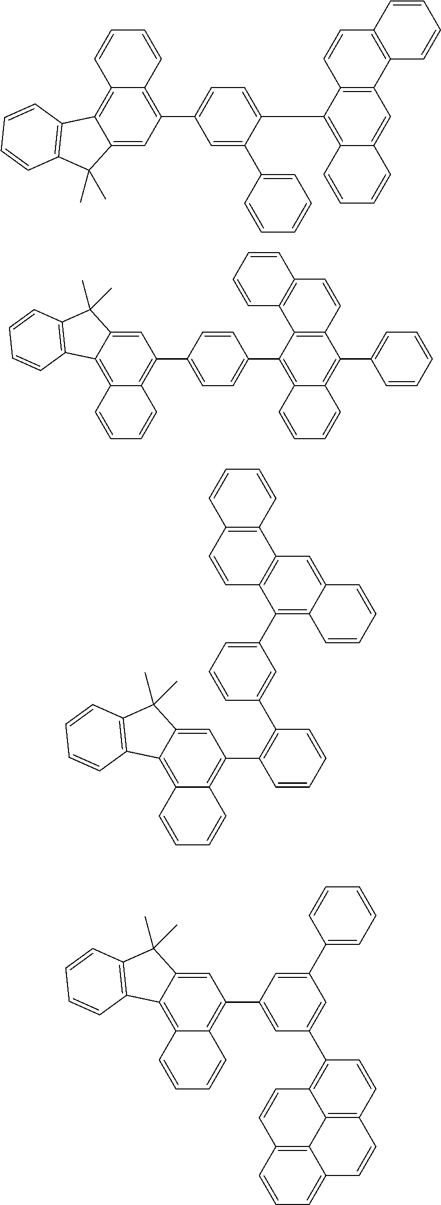

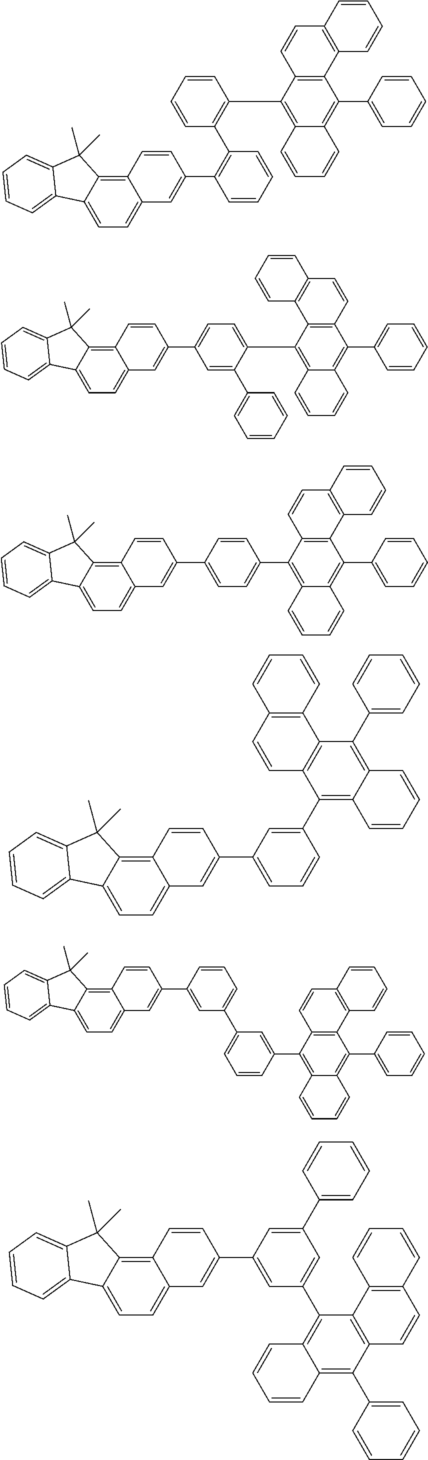

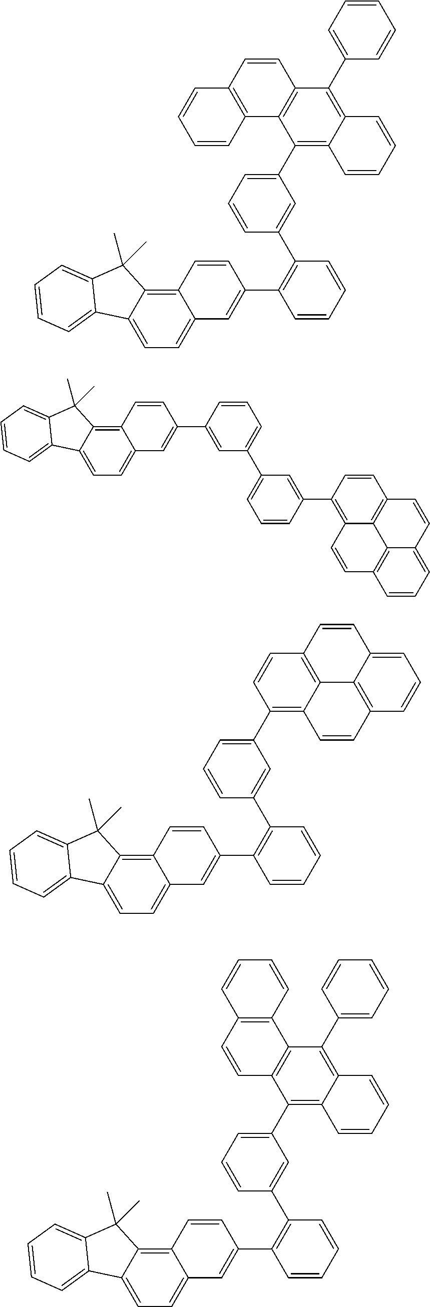

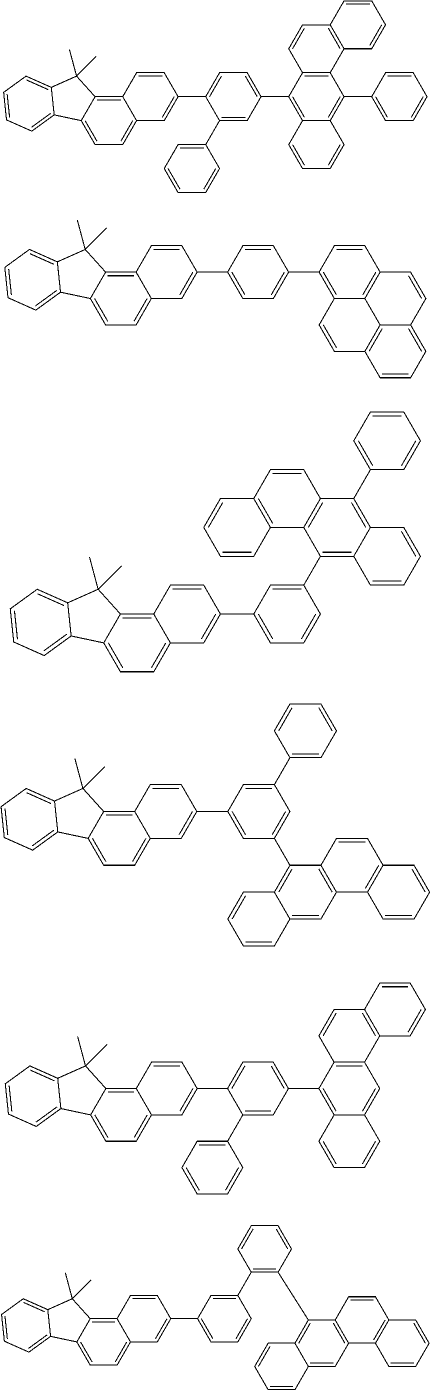

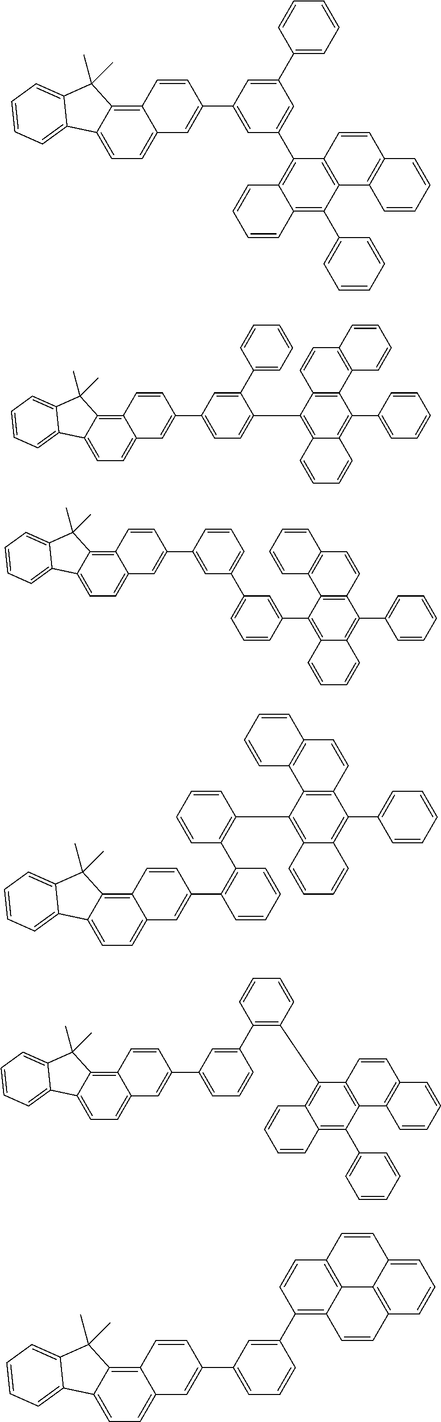

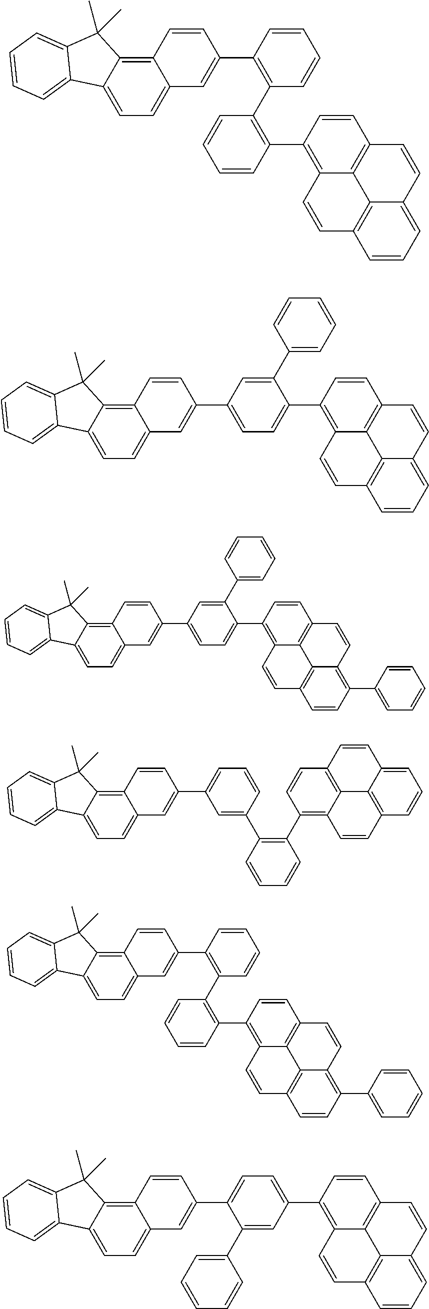

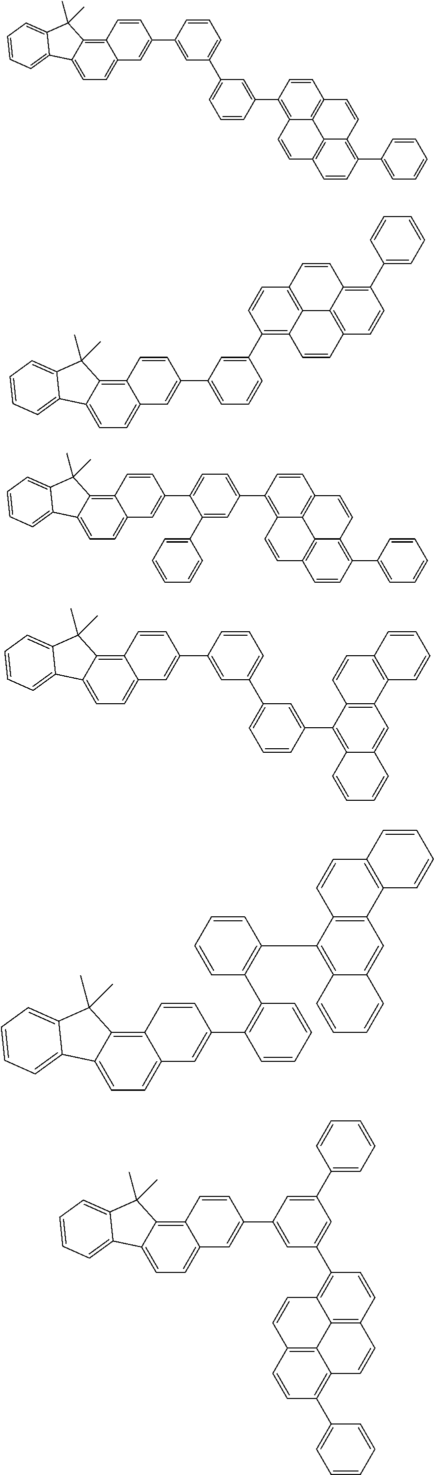

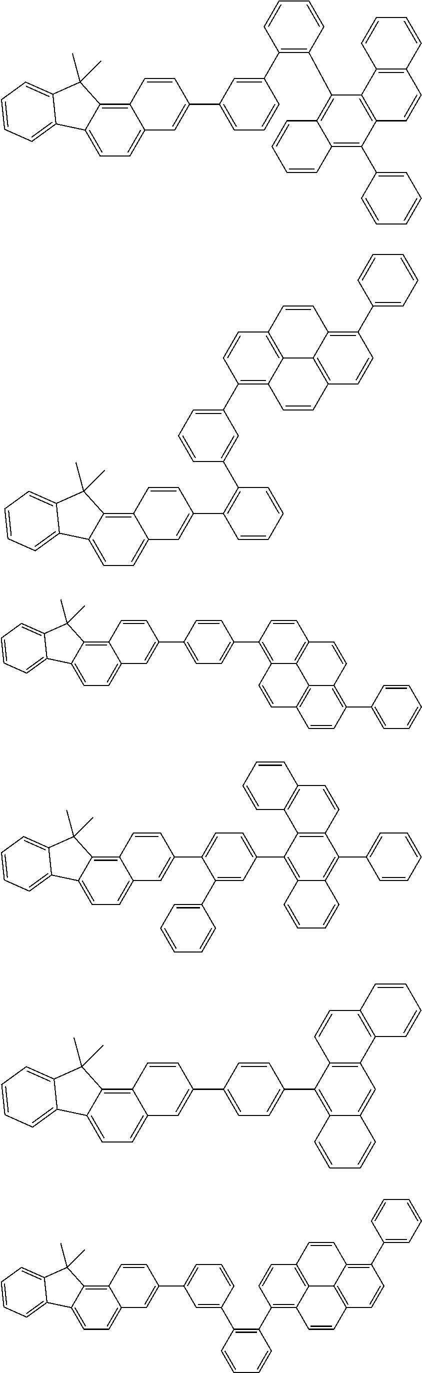

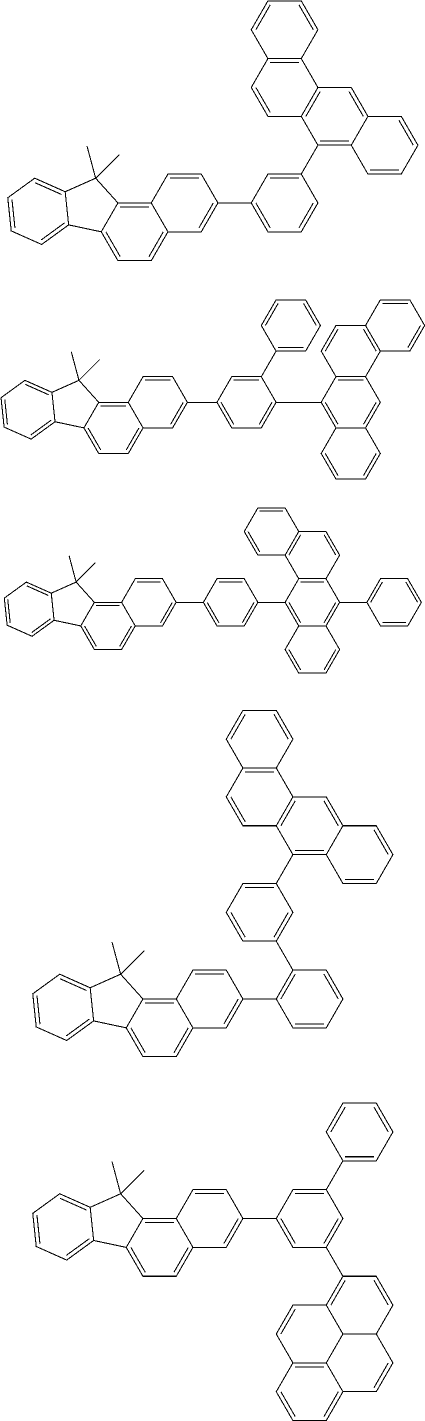

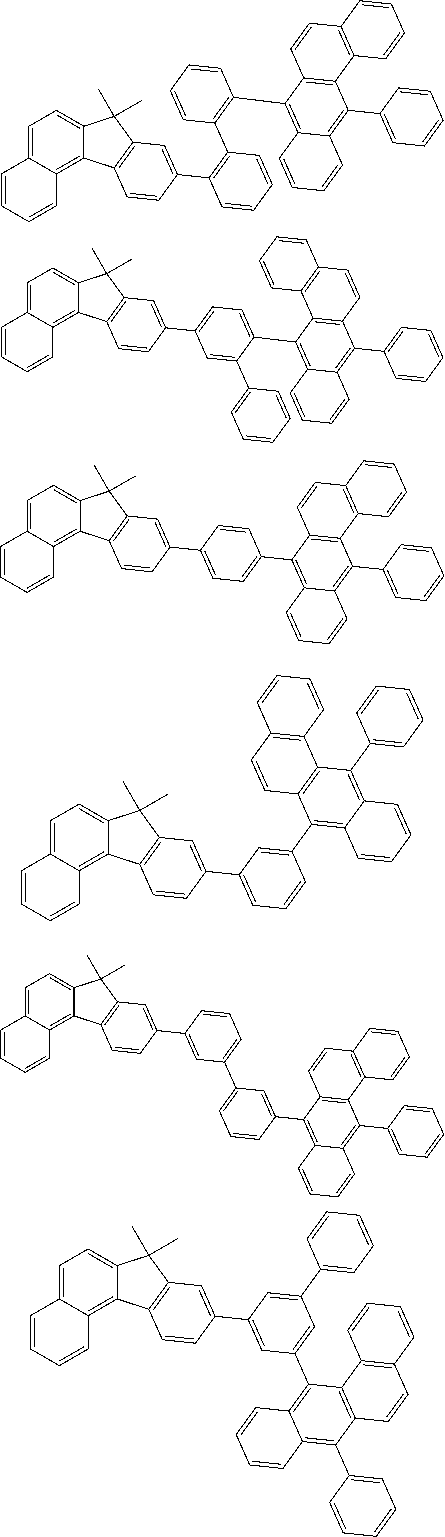

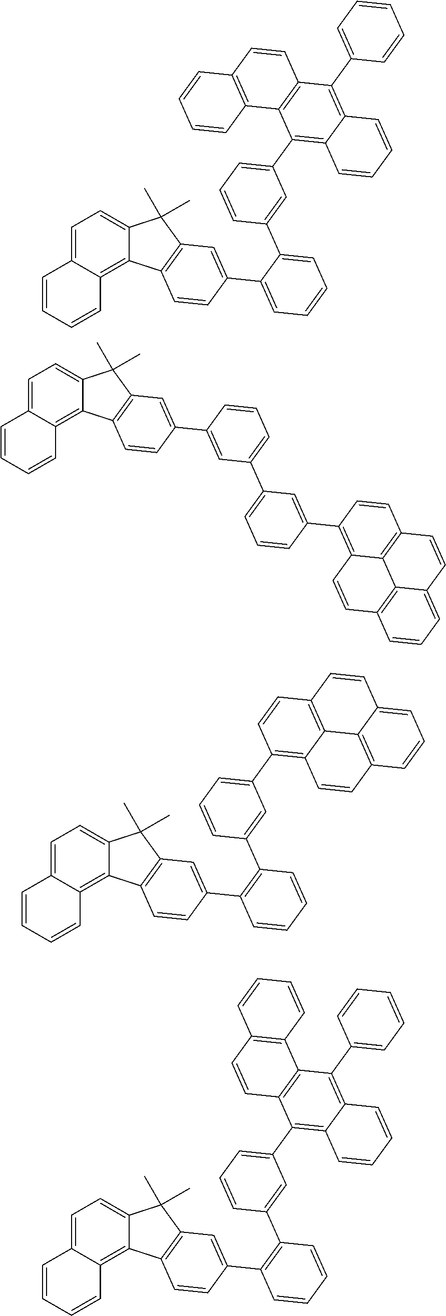

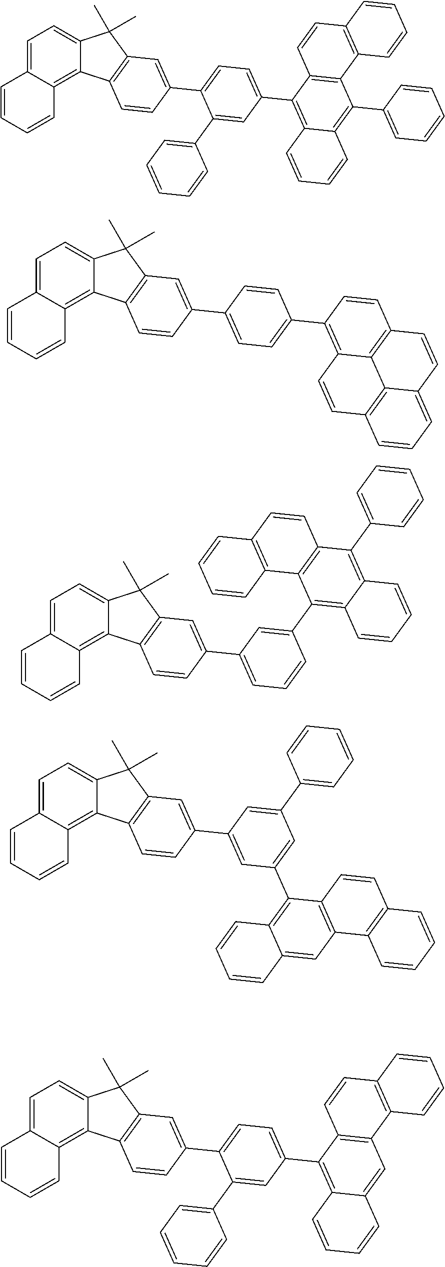

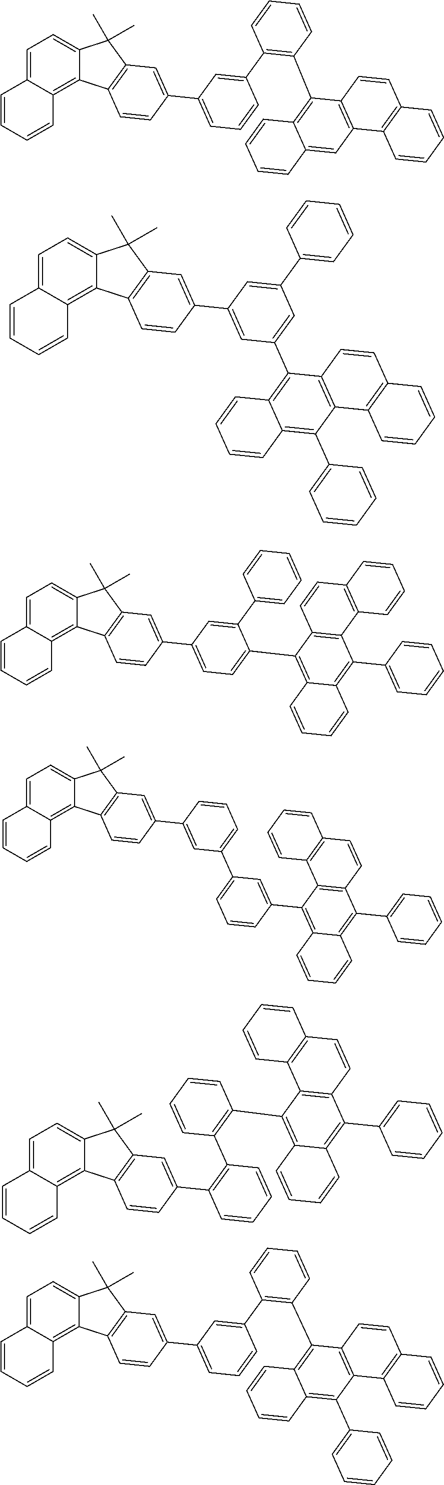

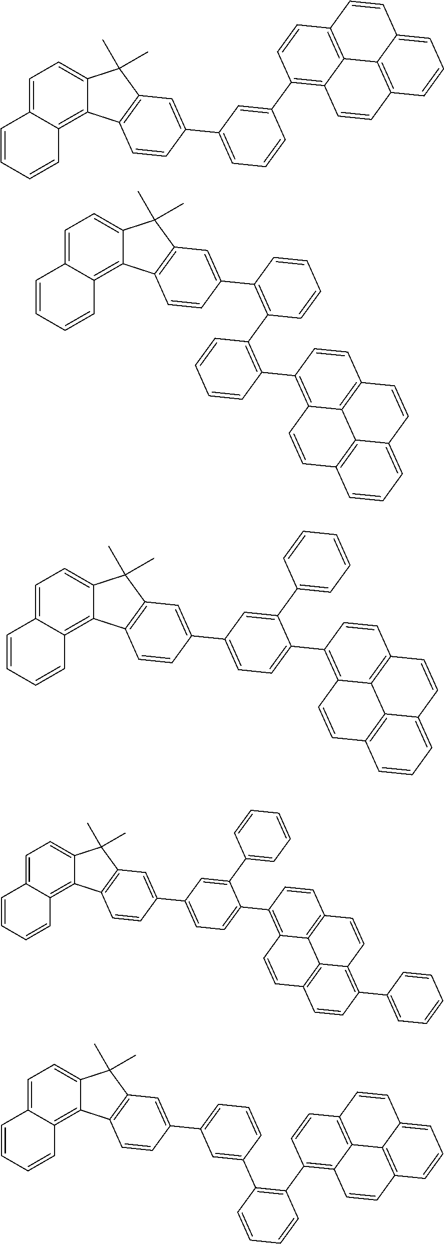

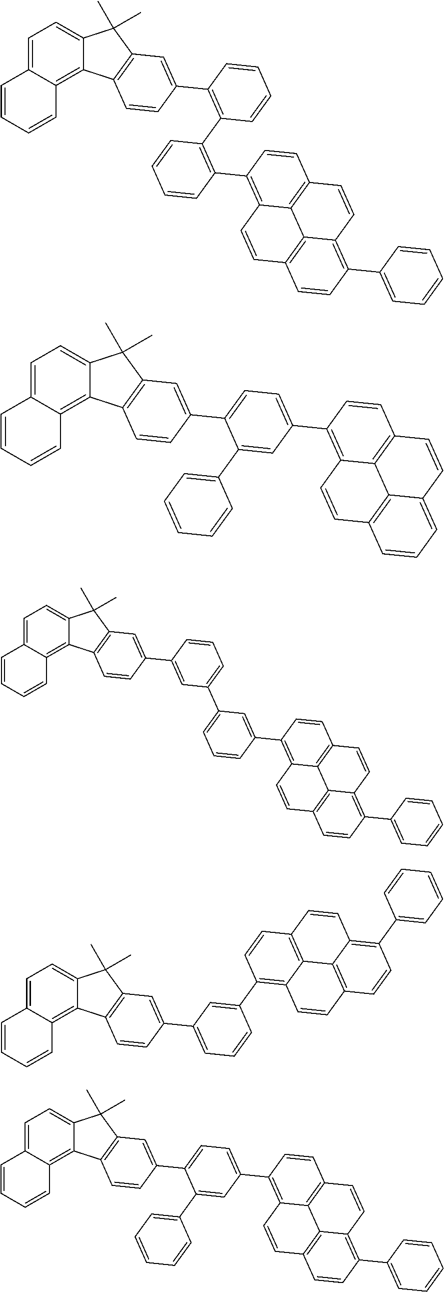

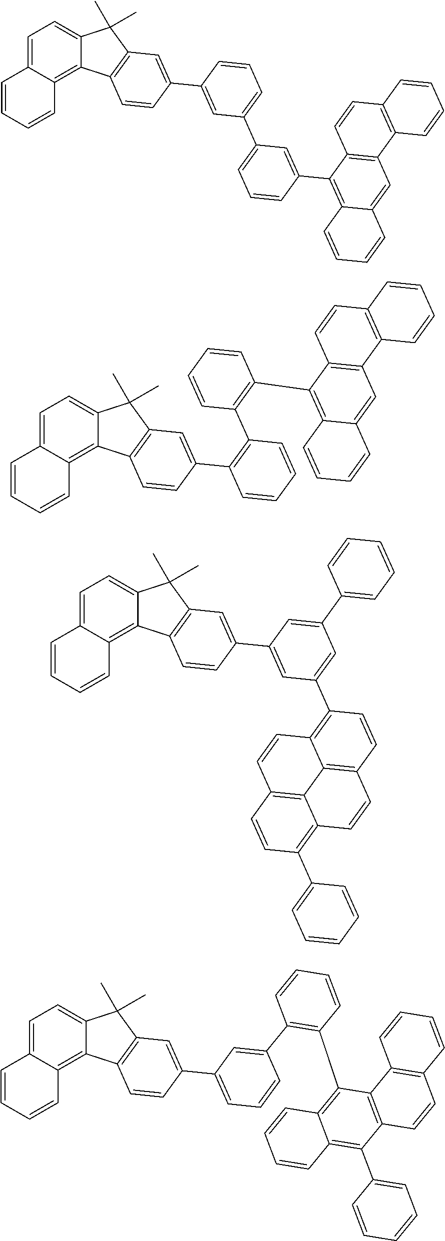

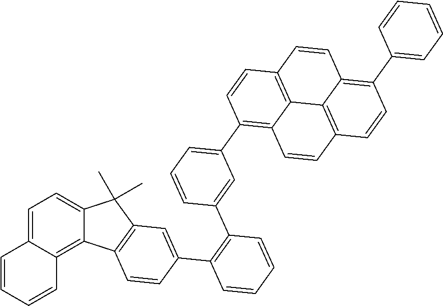 [SMILES: CC1(C)c2cc(-c3ccccc3-c3cccc(-c4ccc5ccc6c(-c7ccccc7)ccc7ccc4c5c76)c3)ccc2-c2c1ccc1ccccc21]

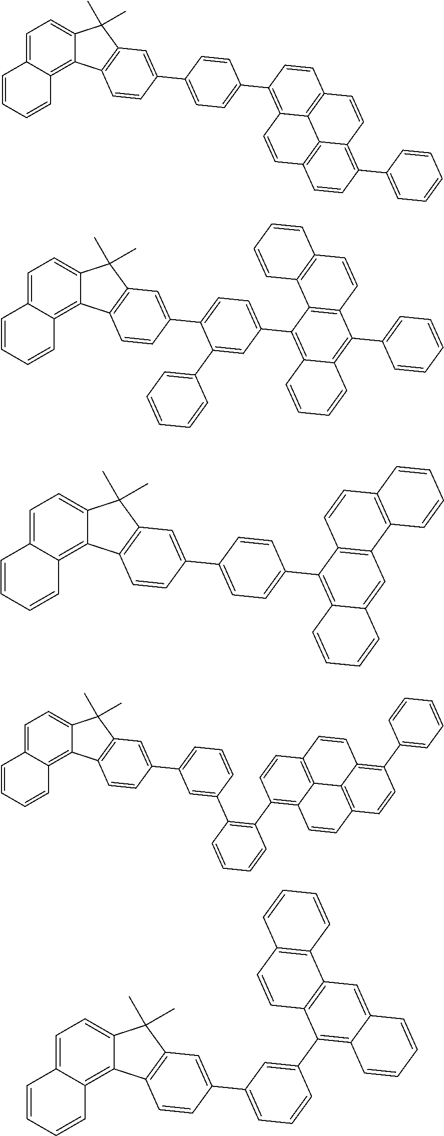

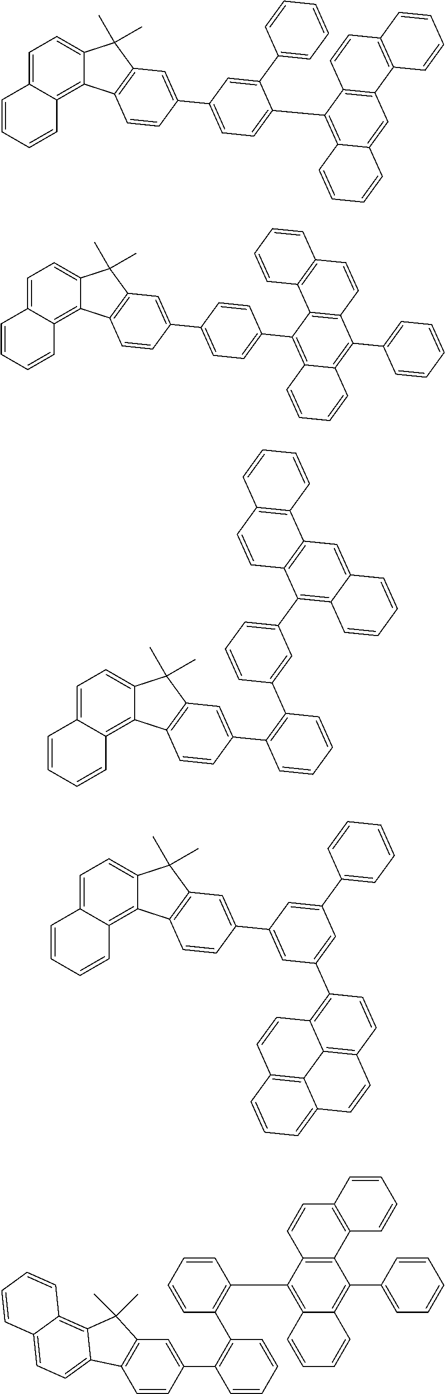

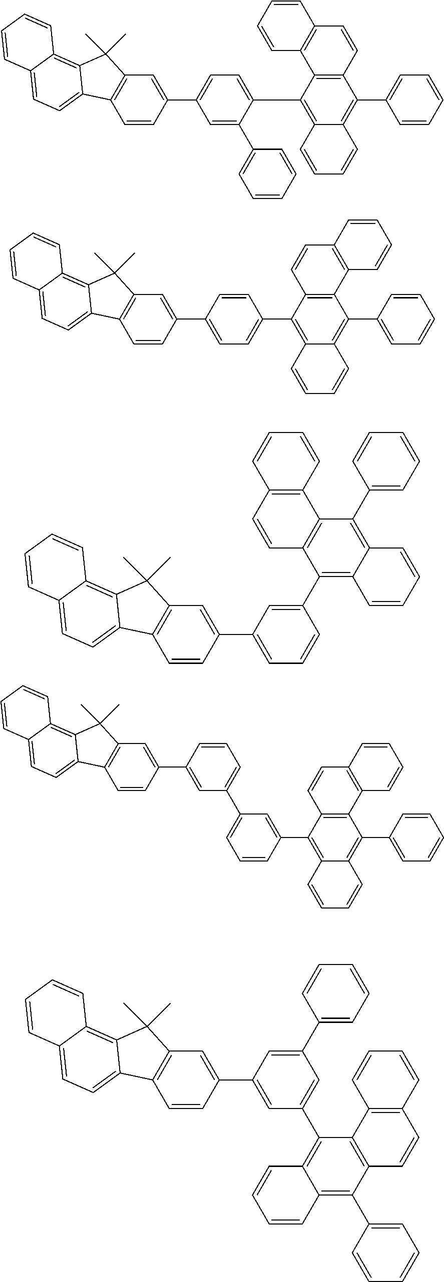

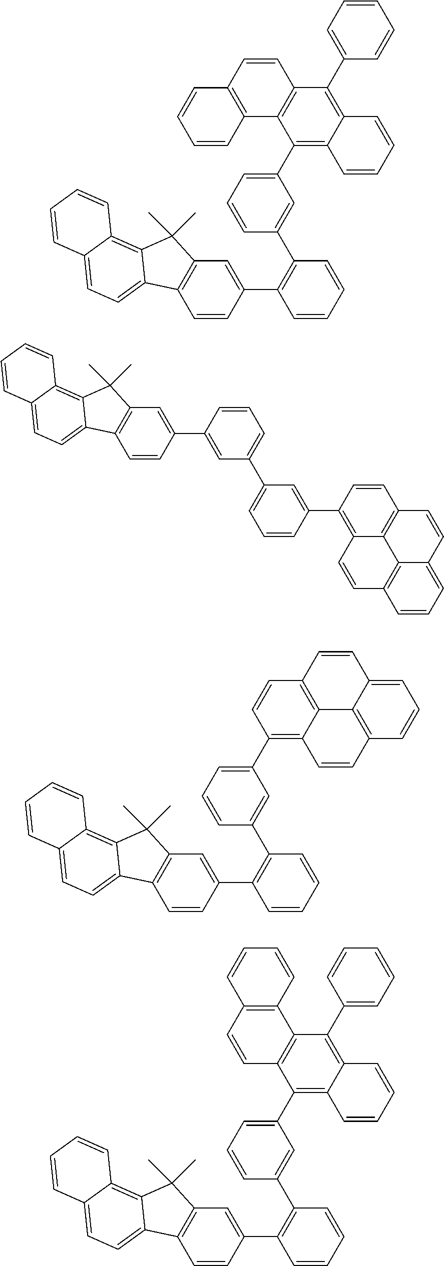

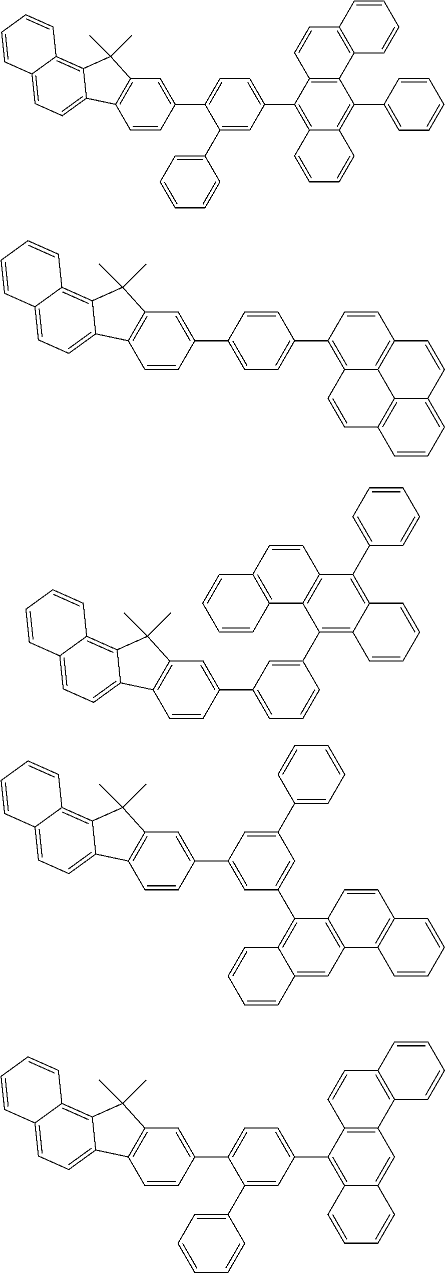

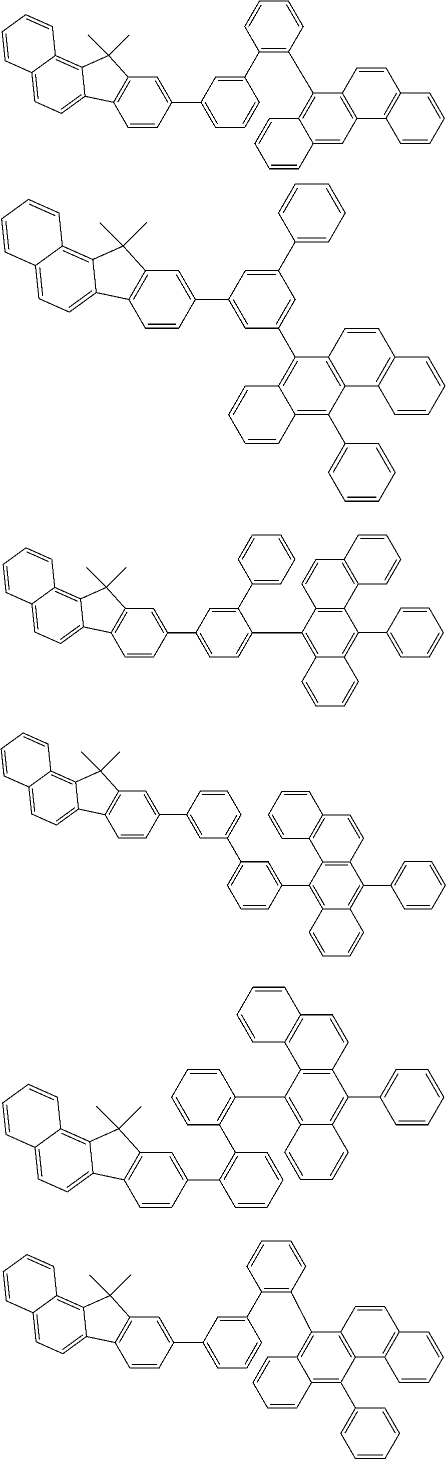

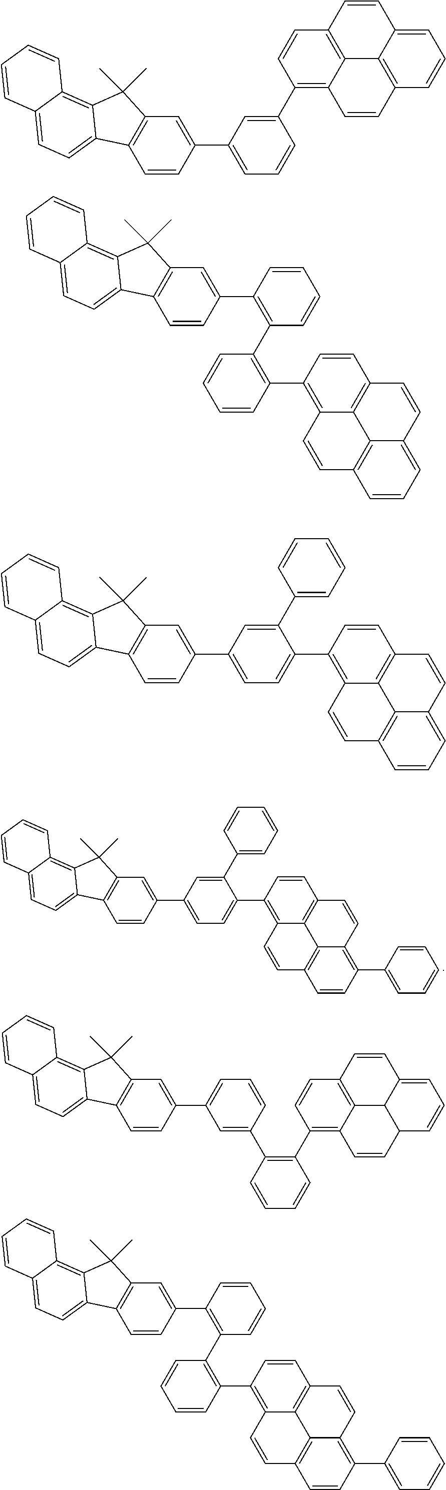

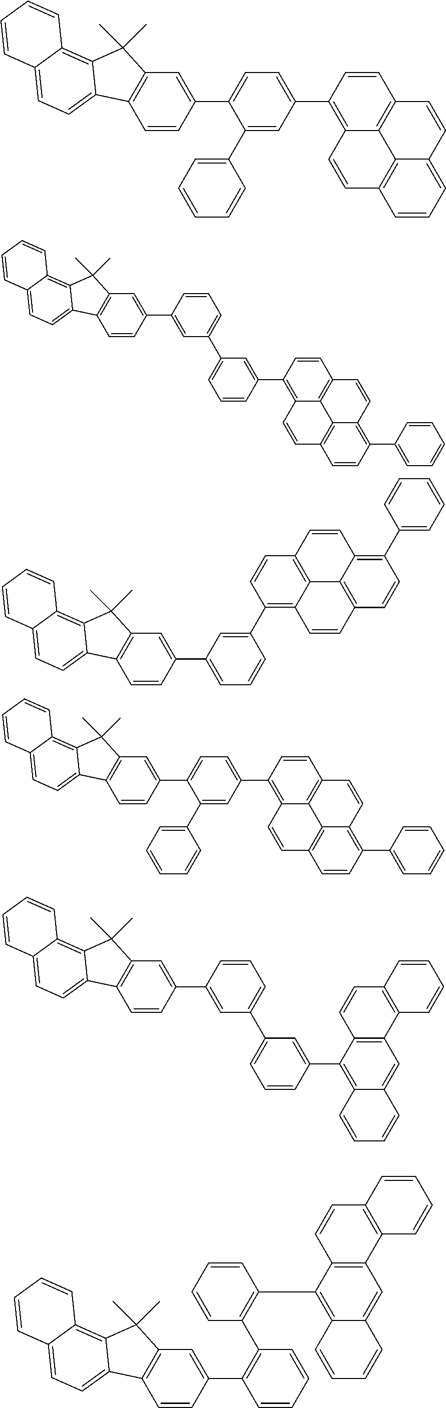

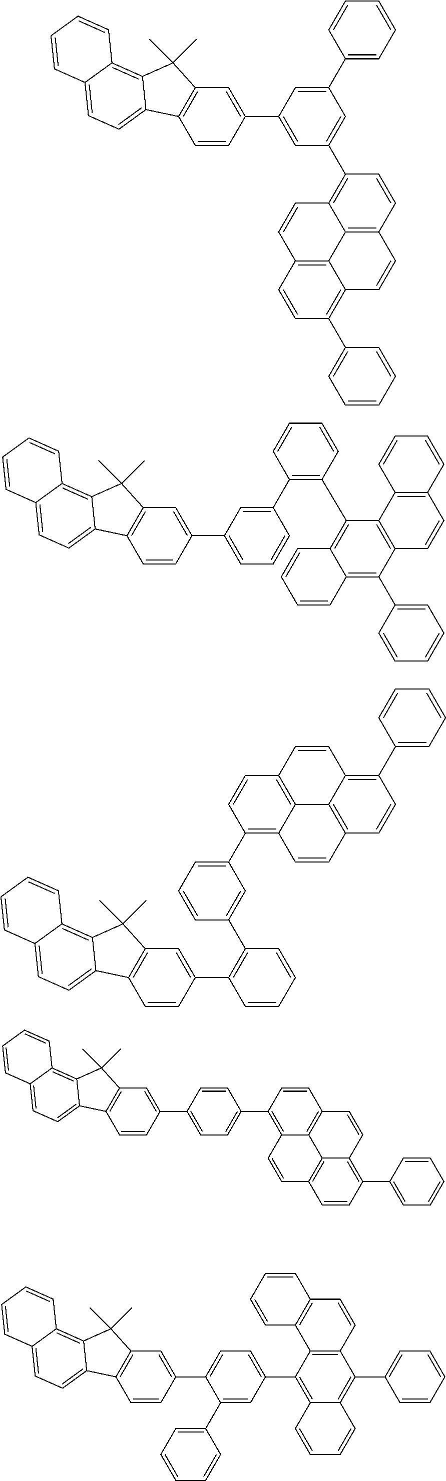

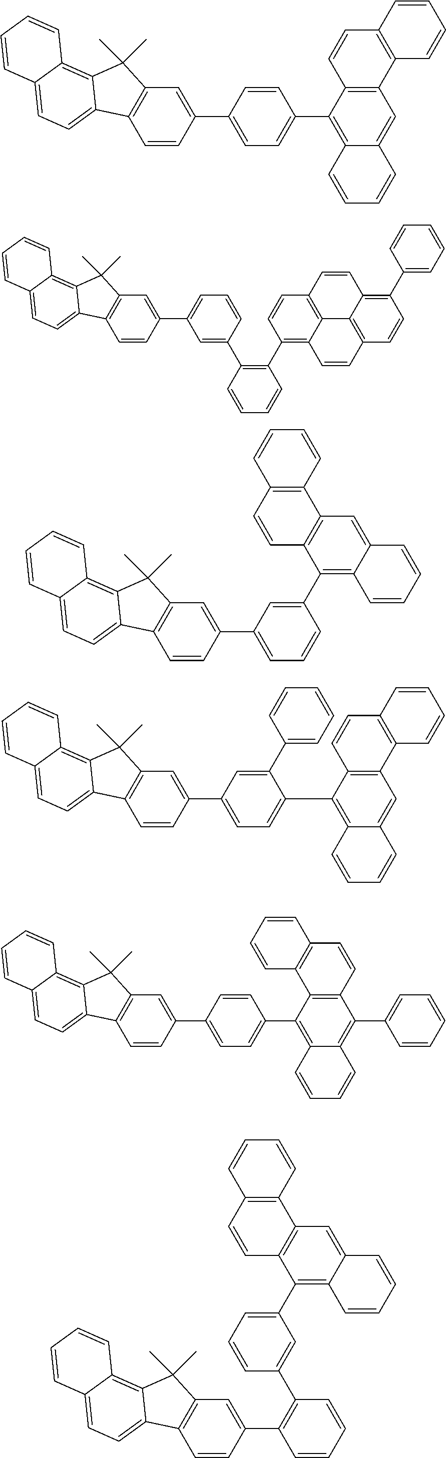

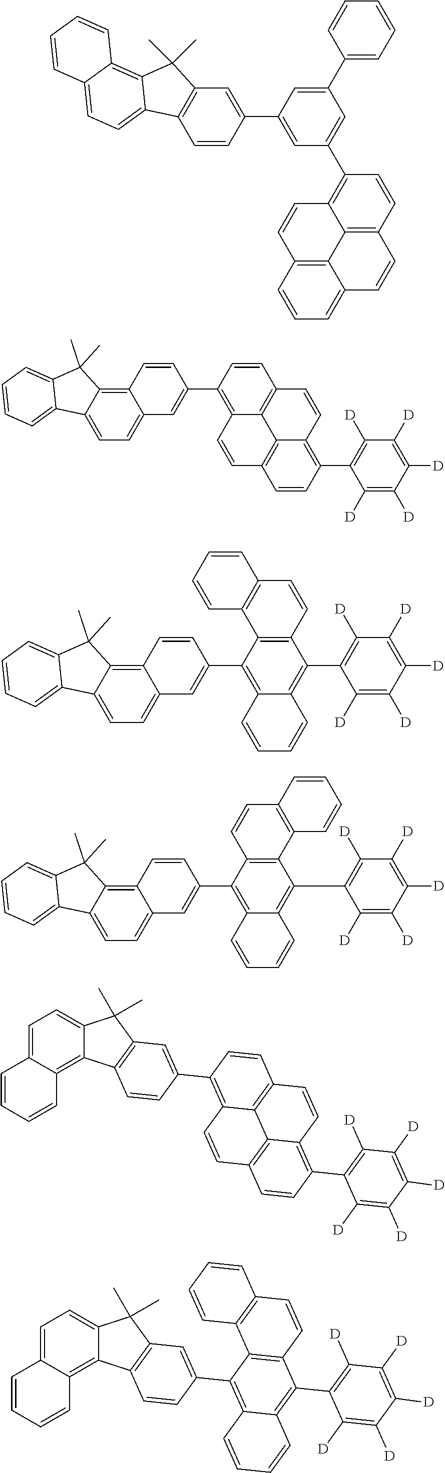

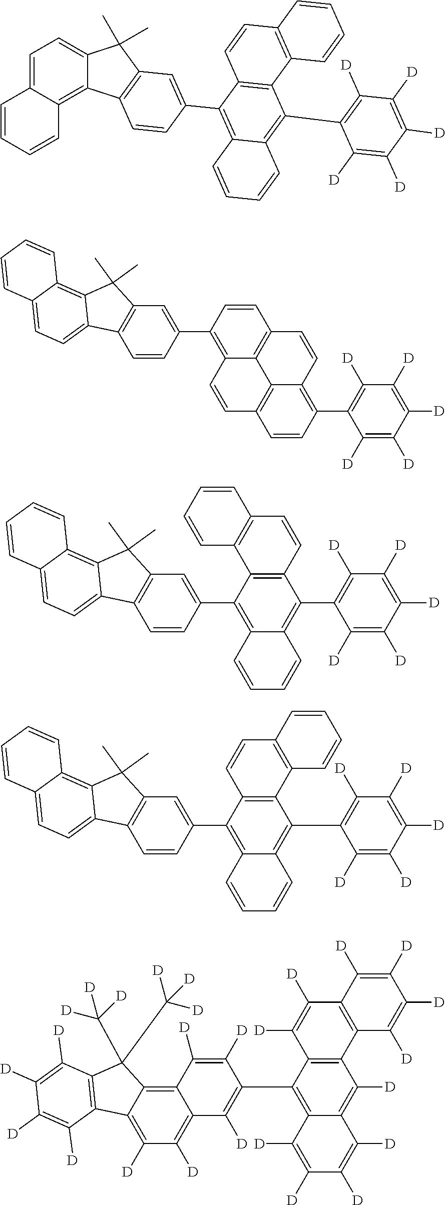

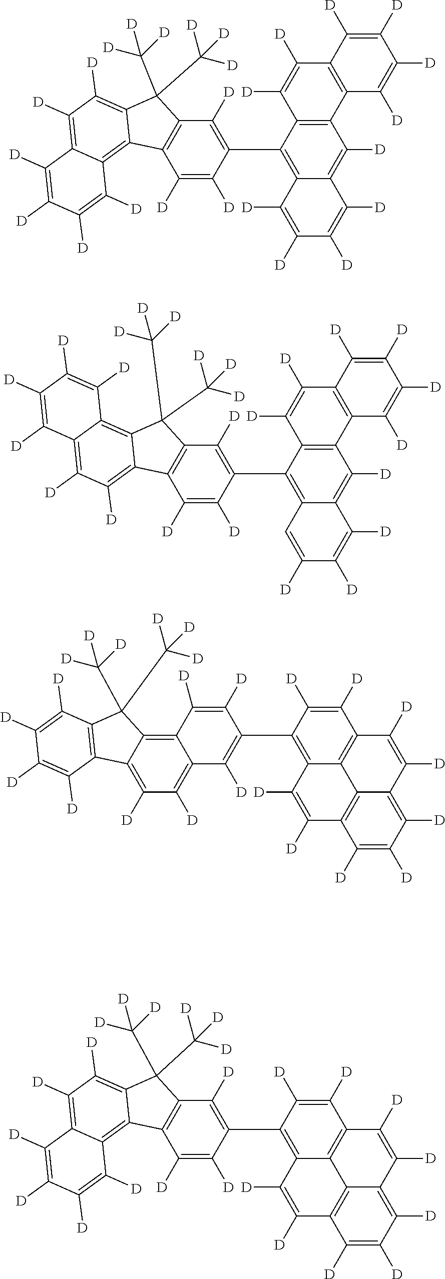

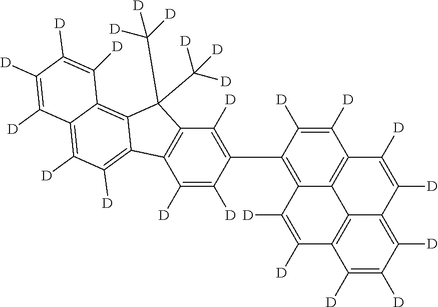 [SMILES: [2H]c1c([2H])c2c(c([2H])c1-c1c([2H])c([2H])c3c([2H])c([2H])c4c([2H])c([2H])c([2H])c5c([2H])c([2H])c1c3c45)C(C([2H])([2H])[2H])(C([2H])([2H])[2H])c1c-2c([2H])c([2H])c2c([2H])c([2H])c([2H])c([2H])c12]

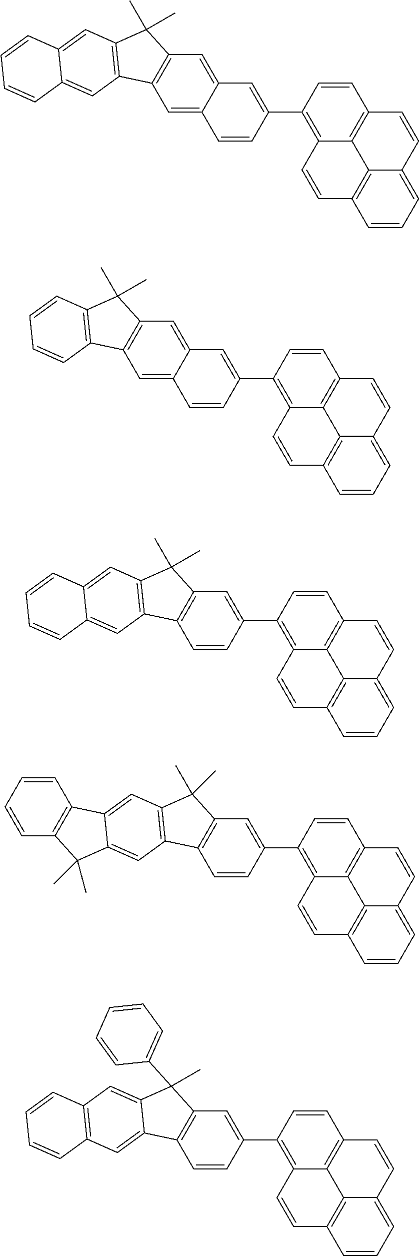

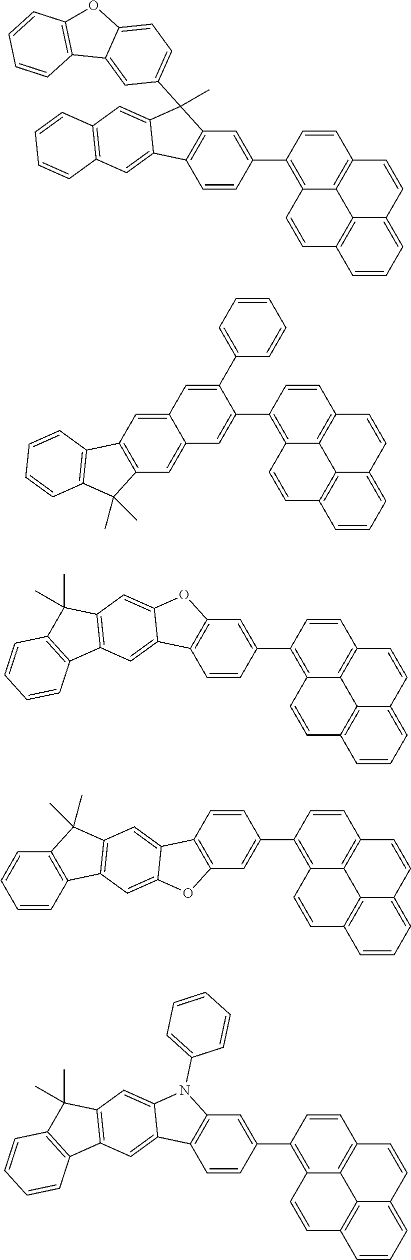

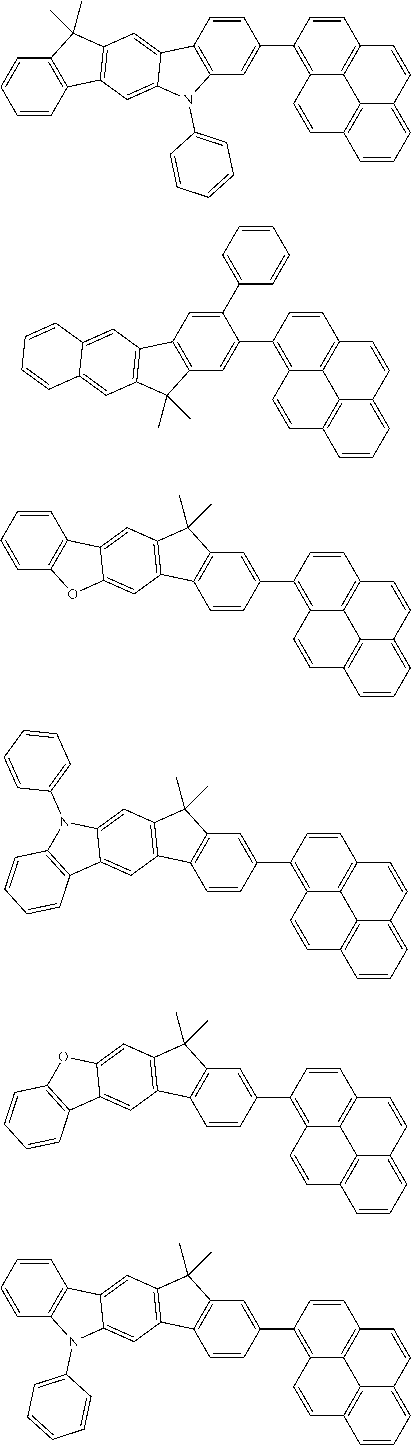

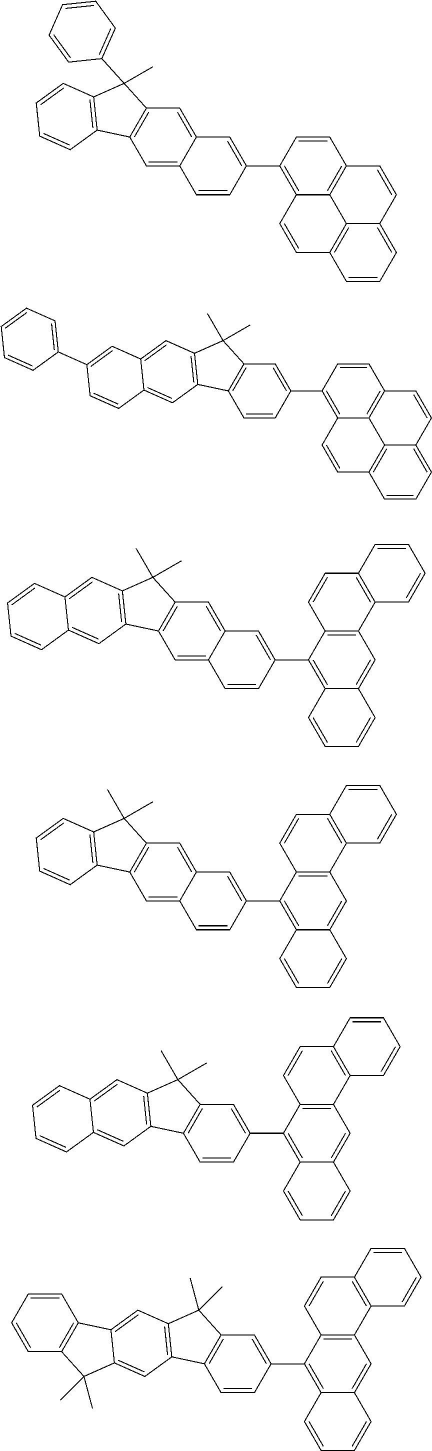

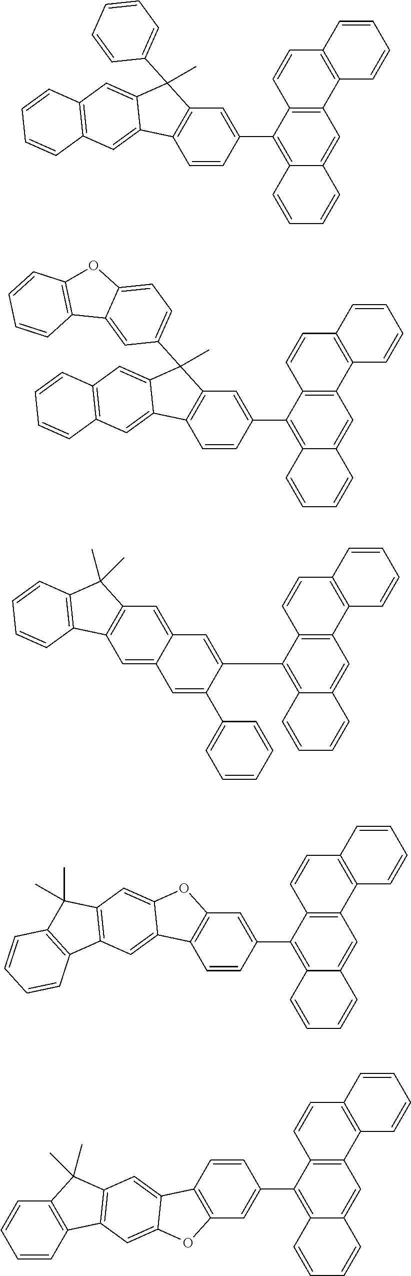

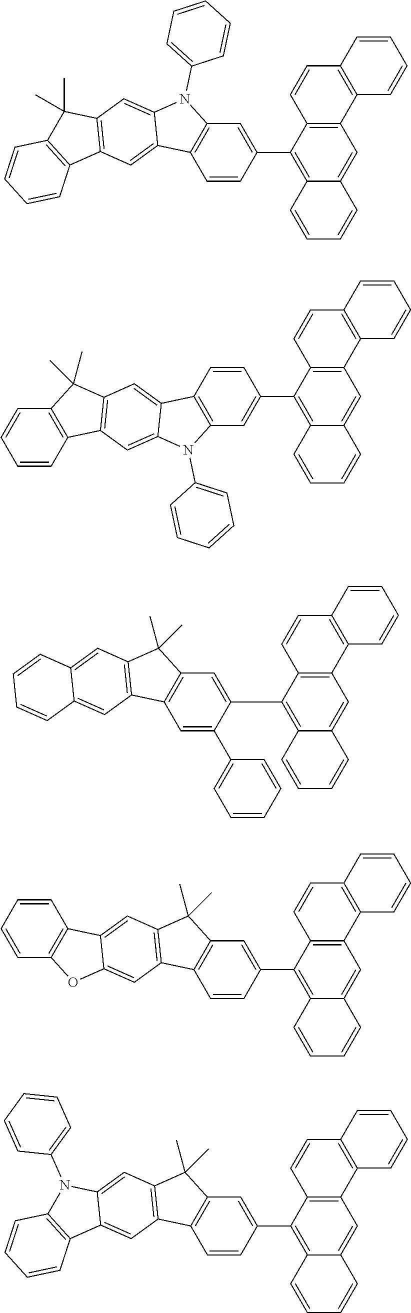

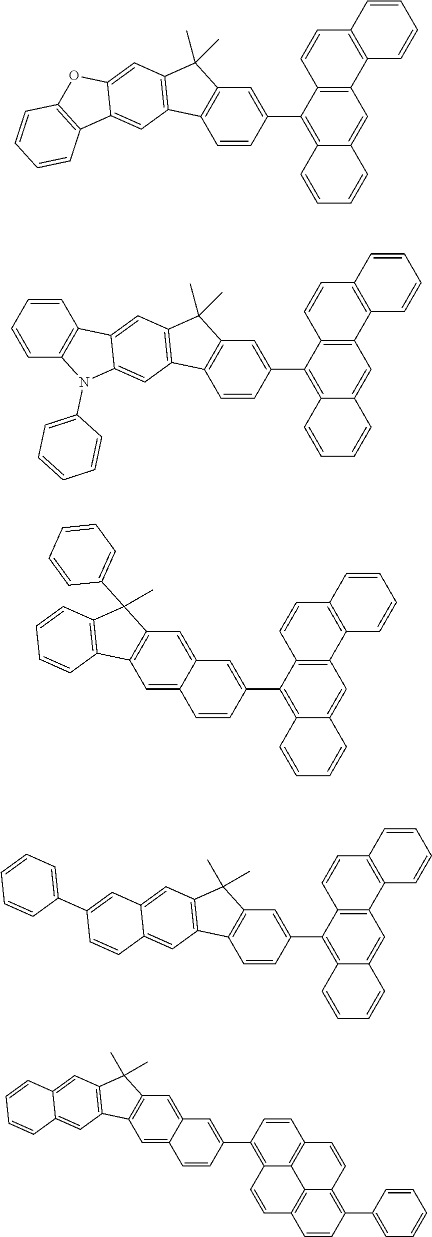

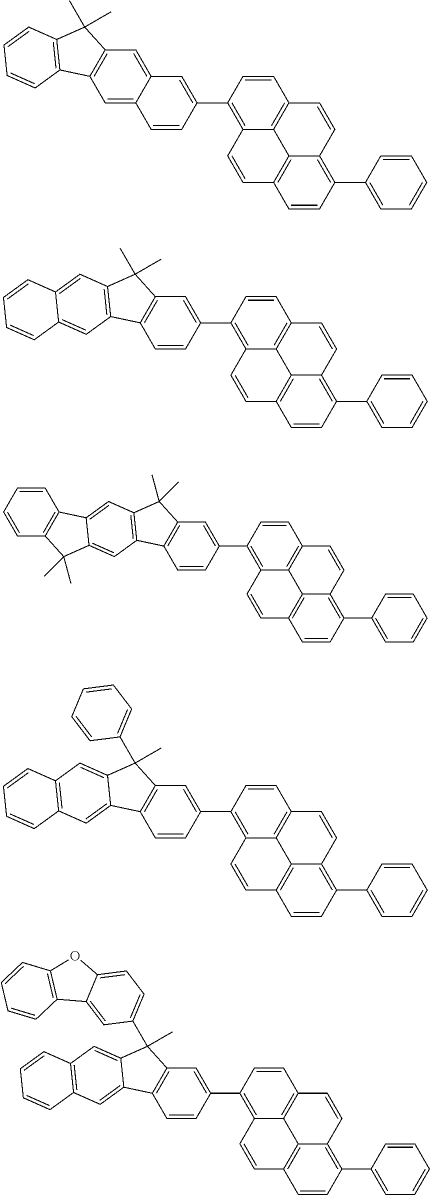

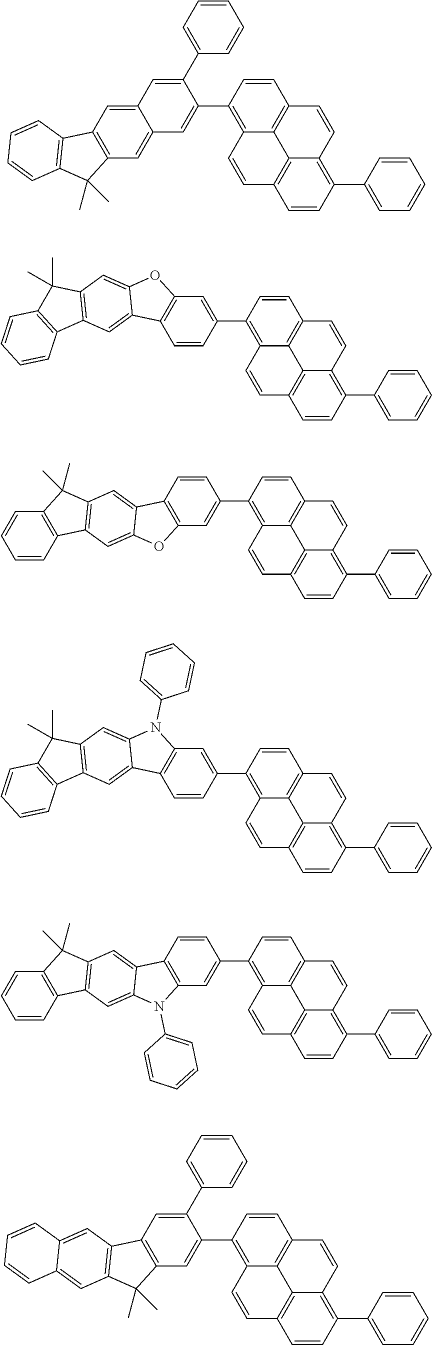

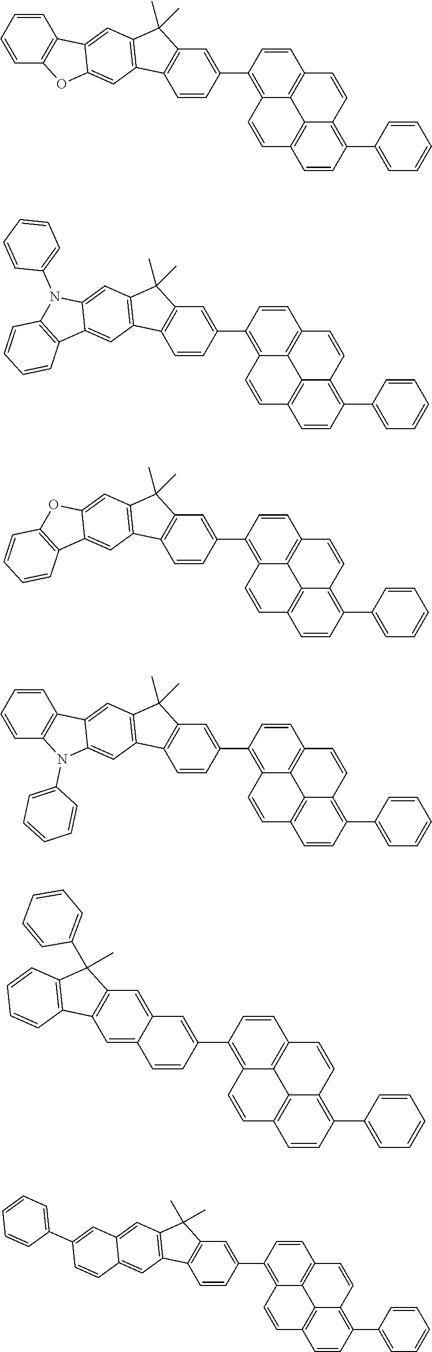

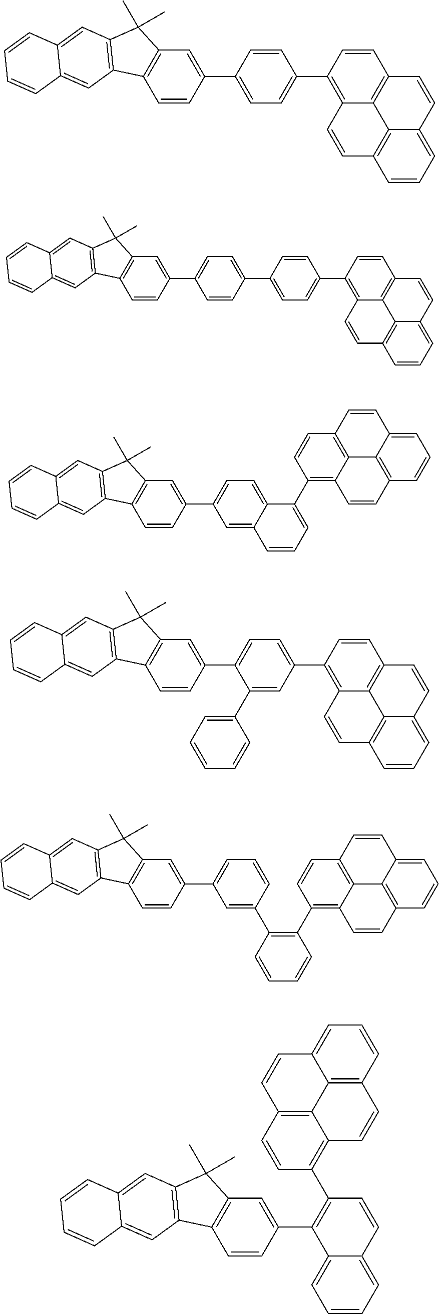

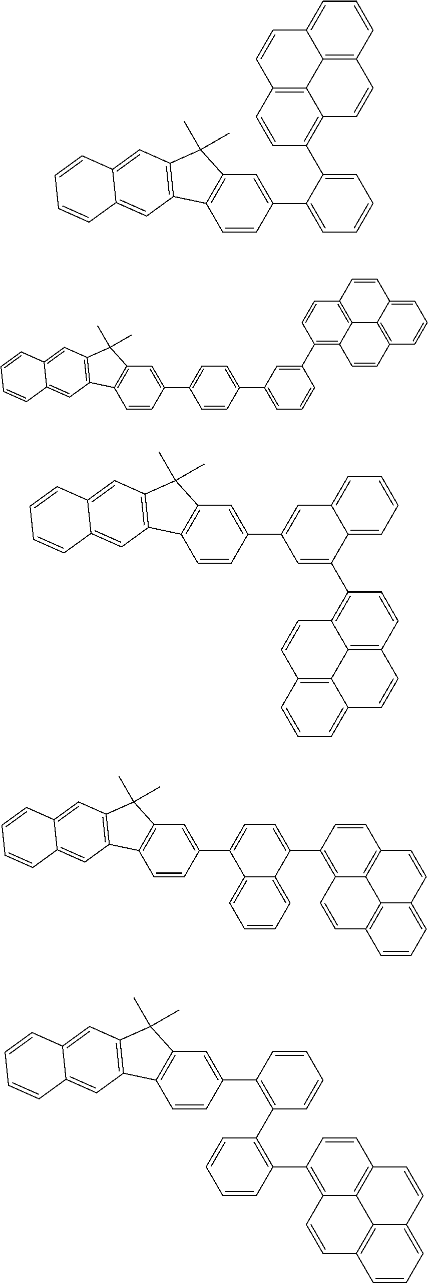

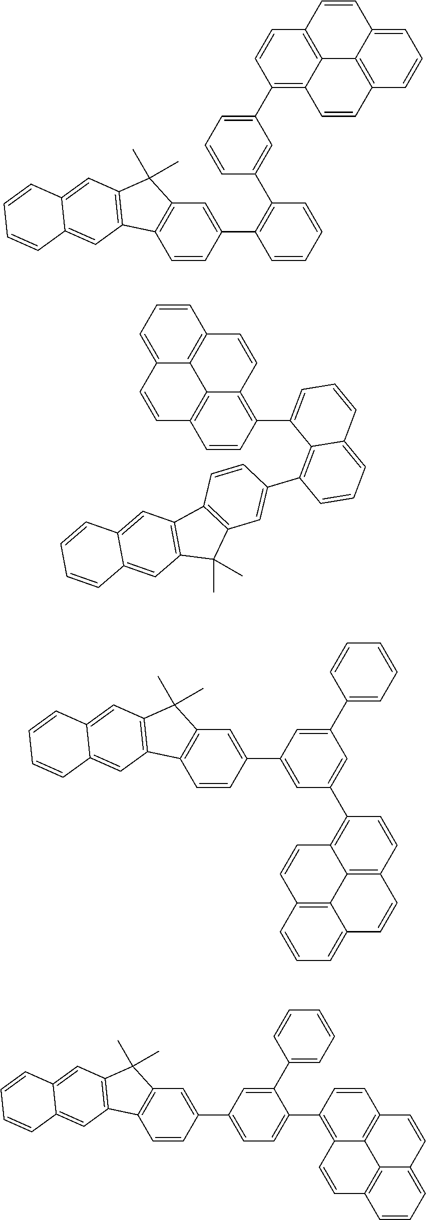

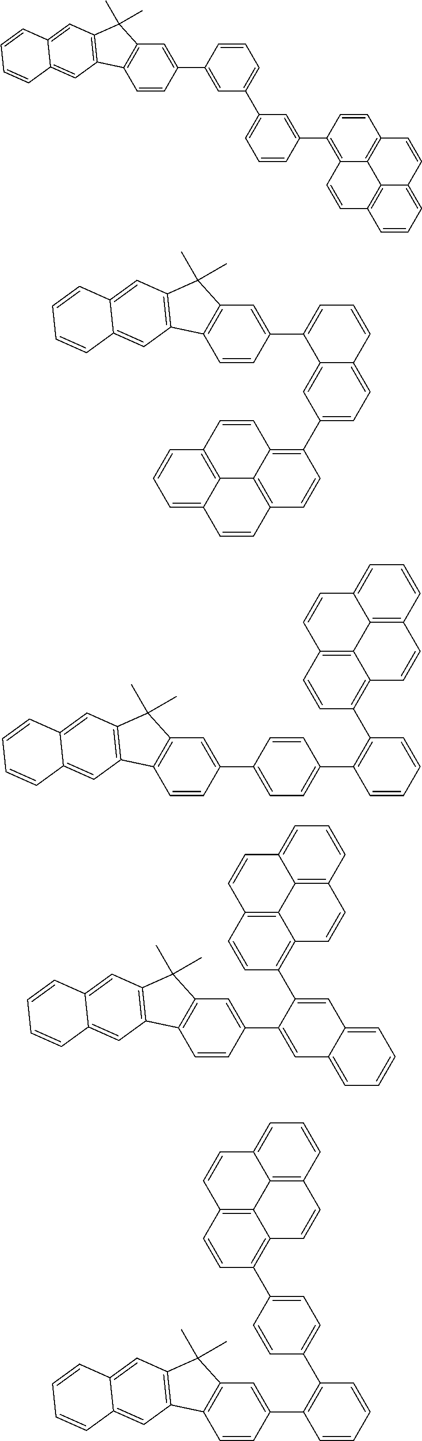

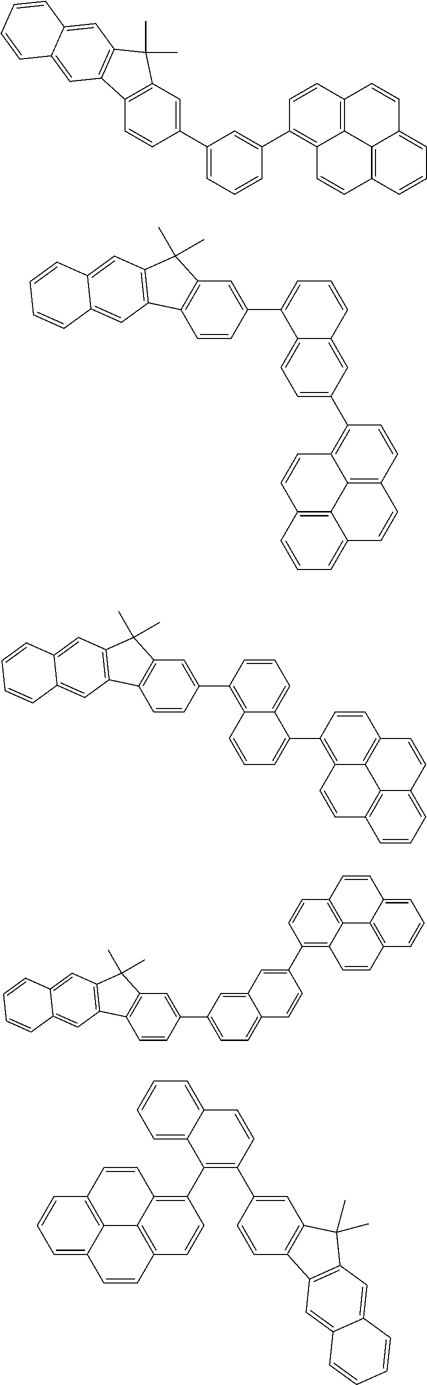

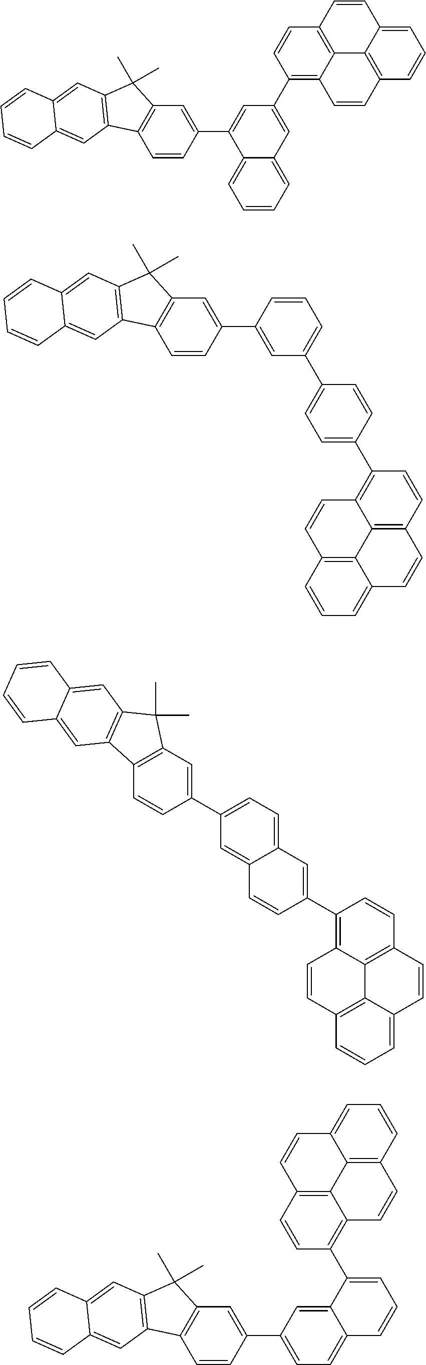

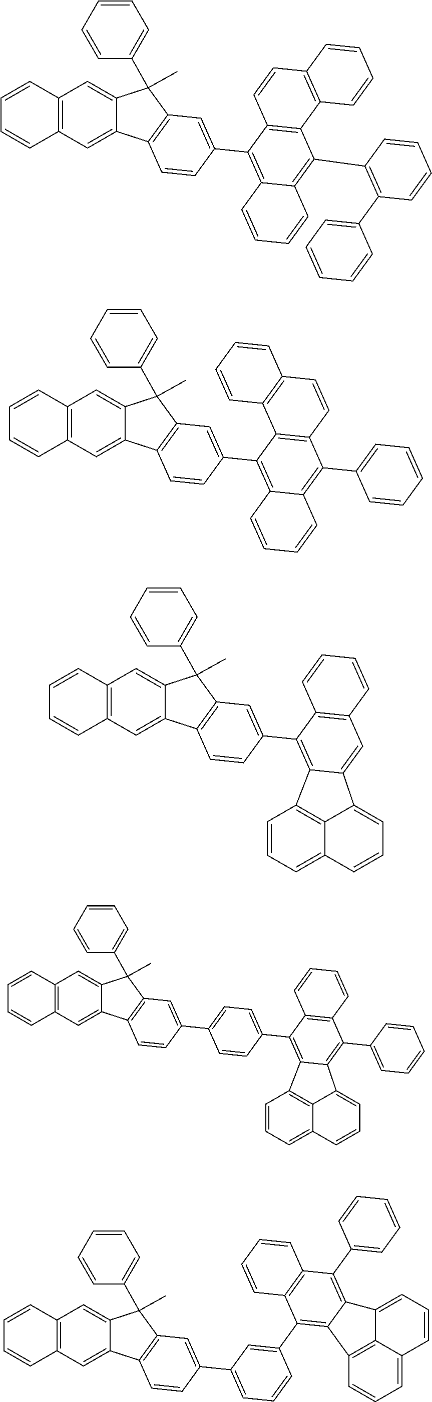

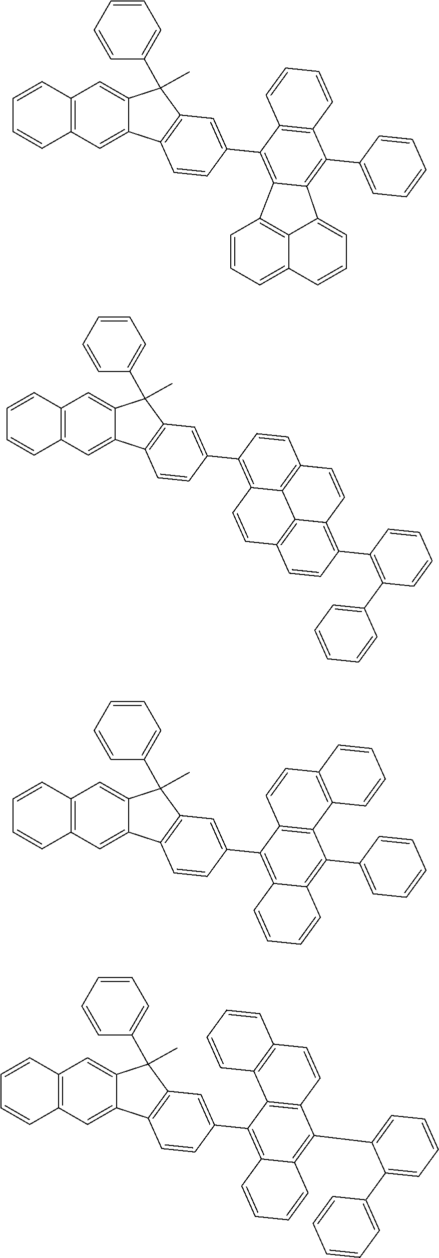

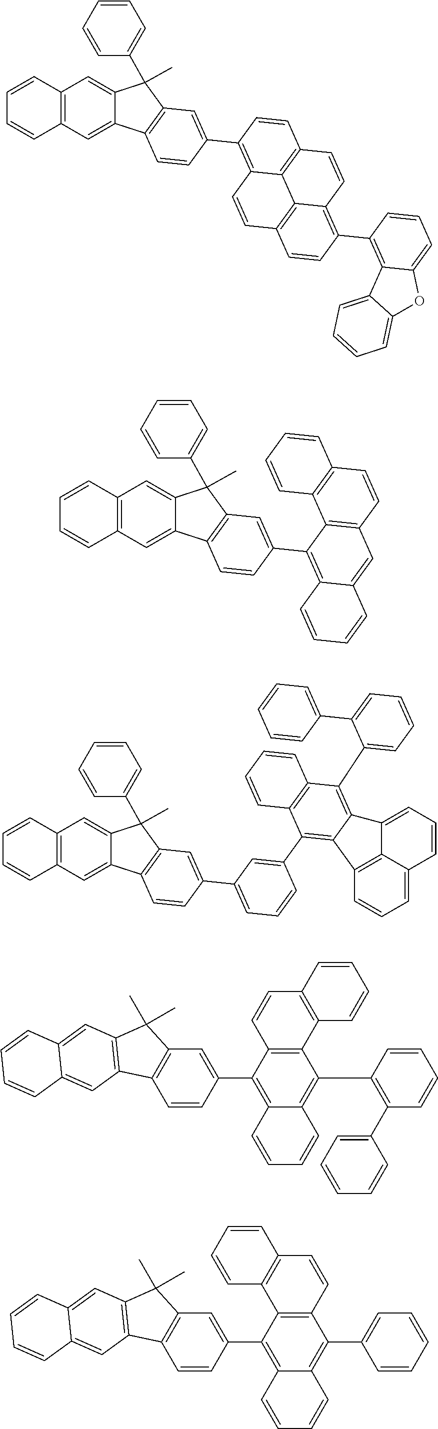

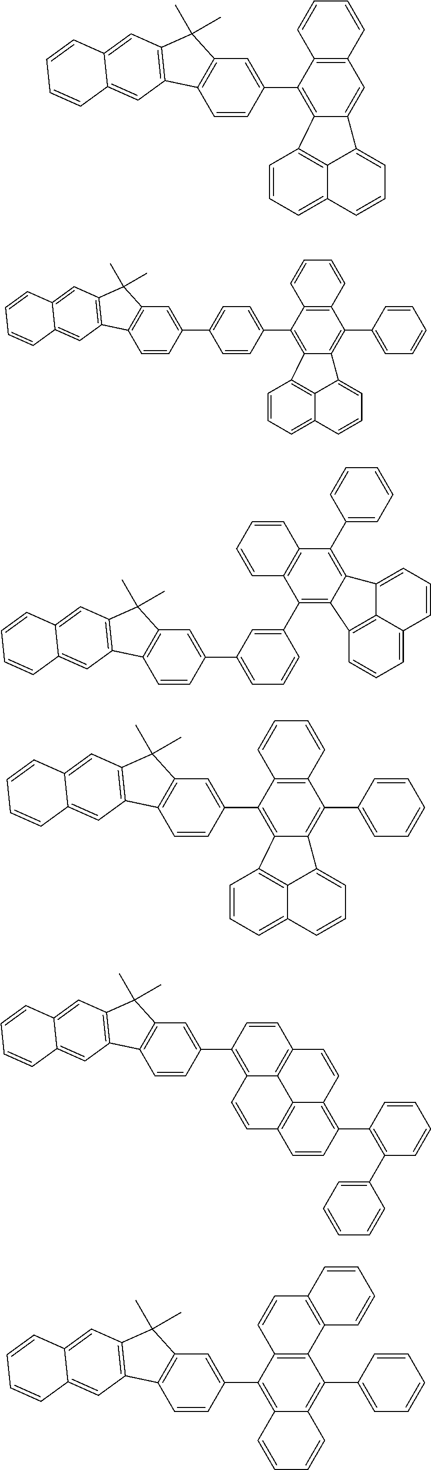

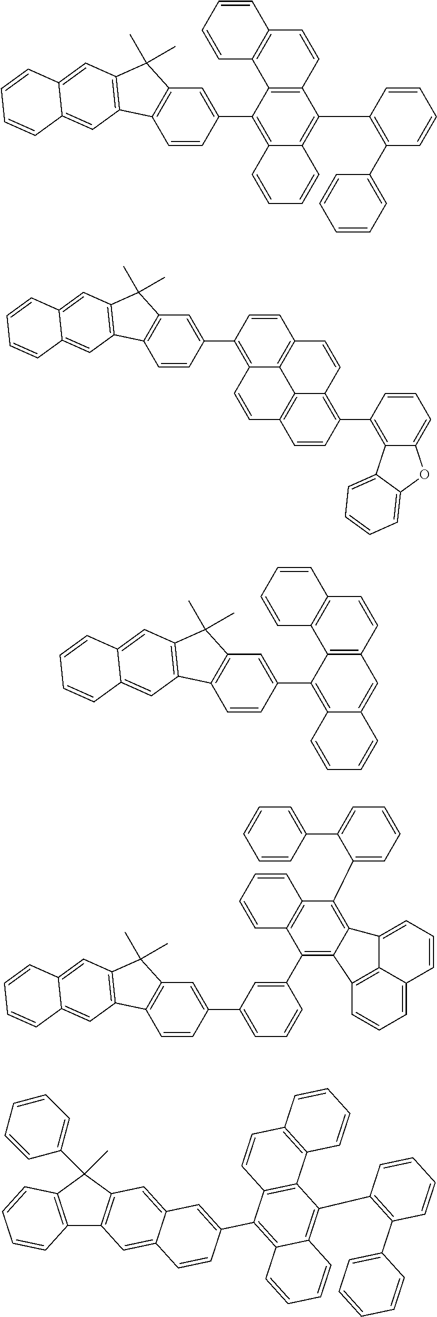

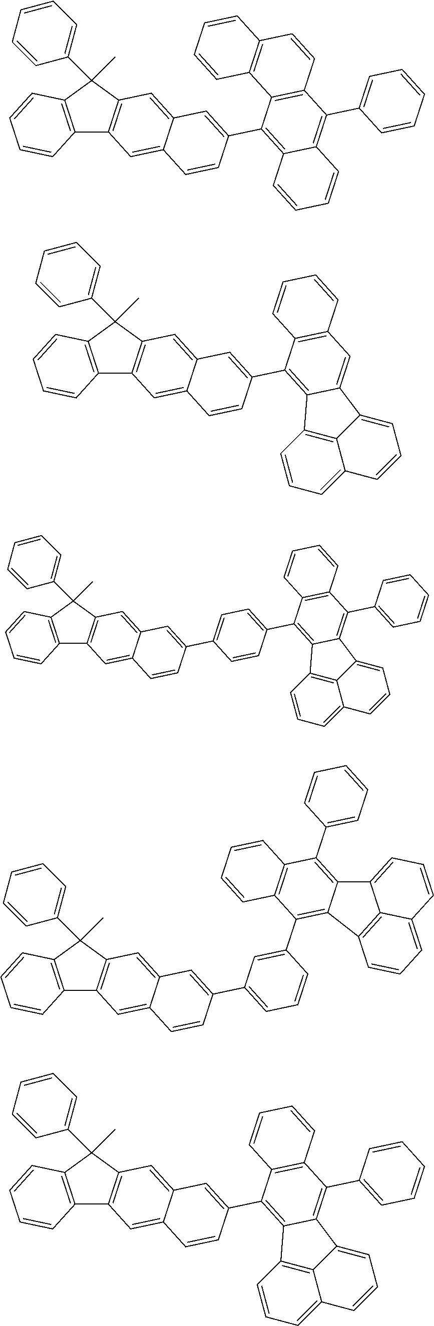

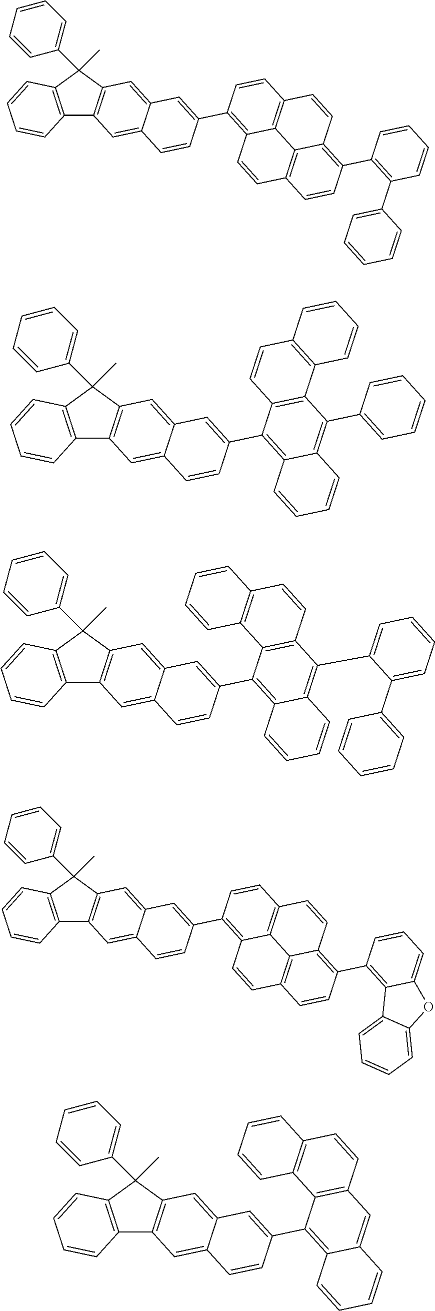

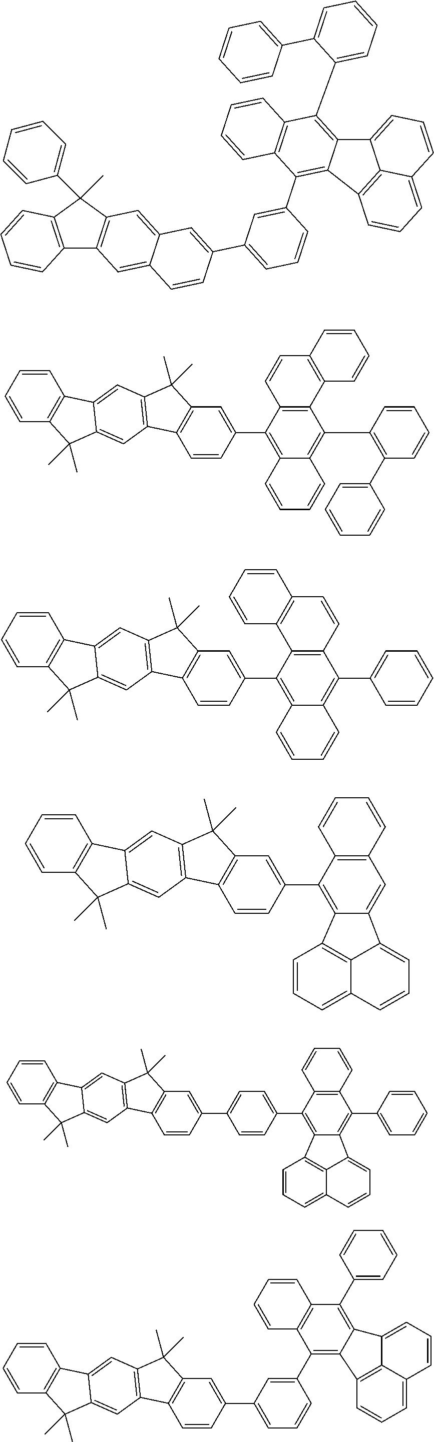

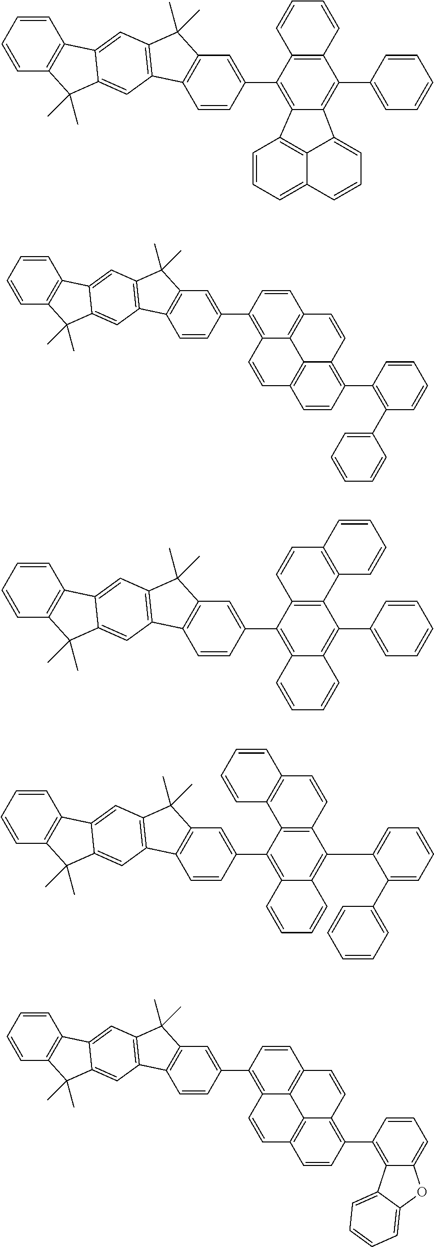

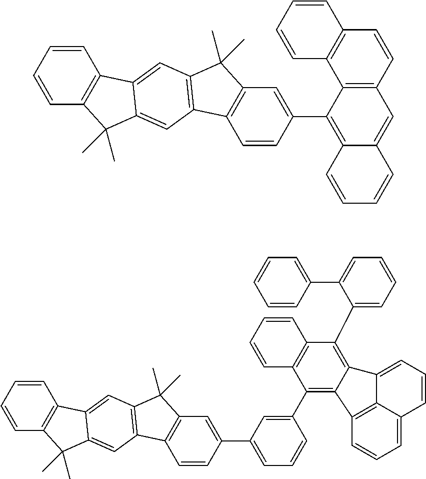

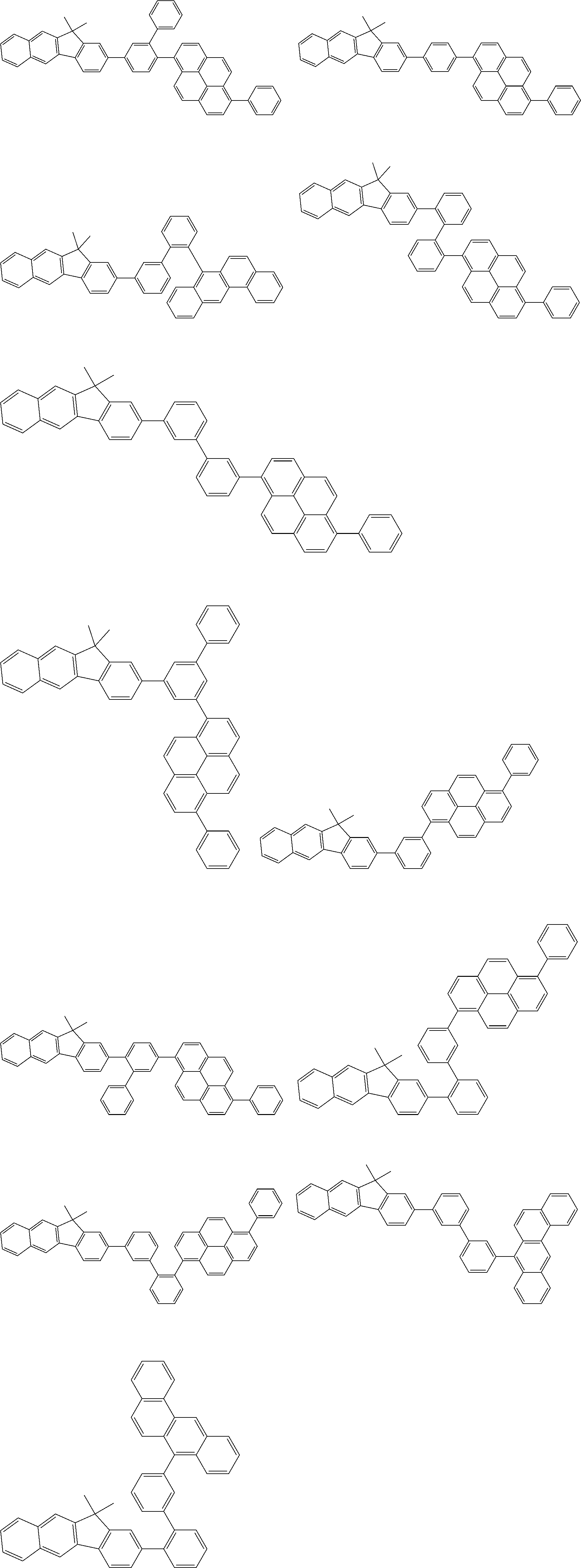

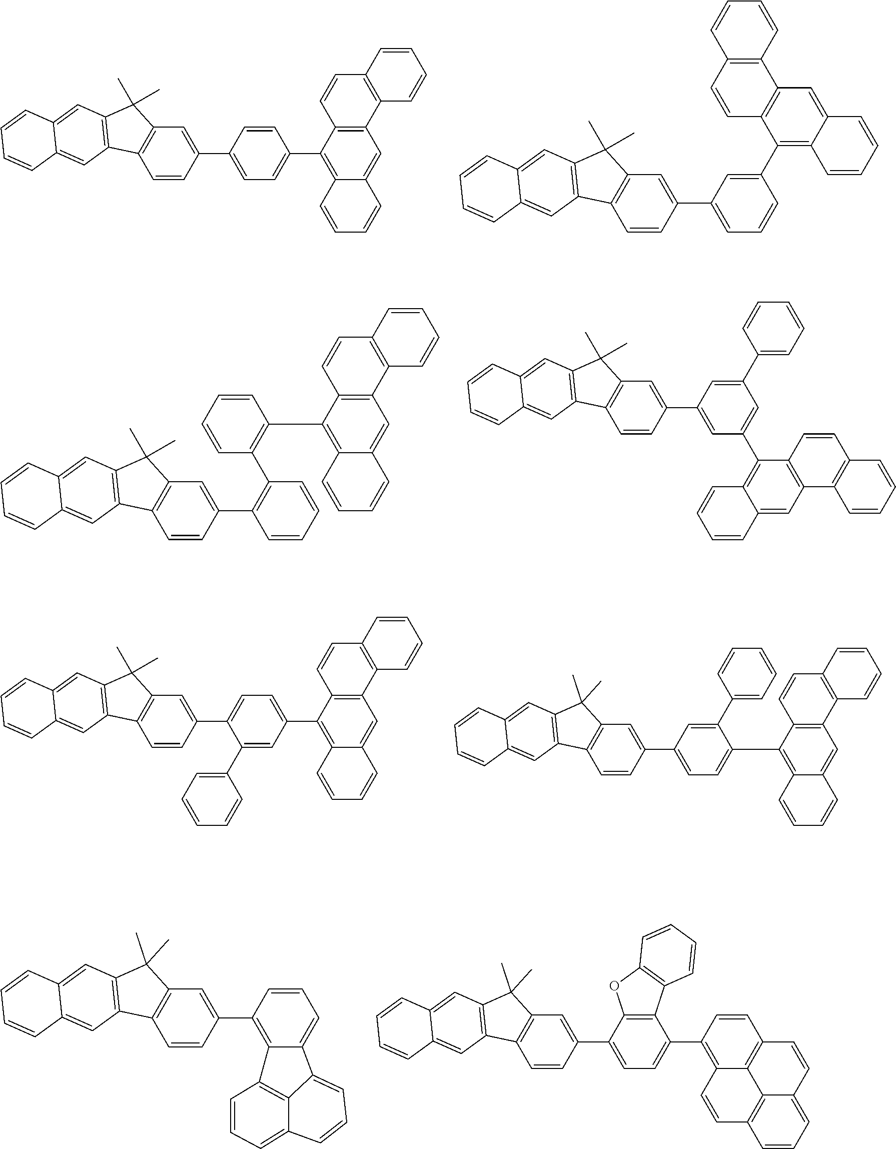

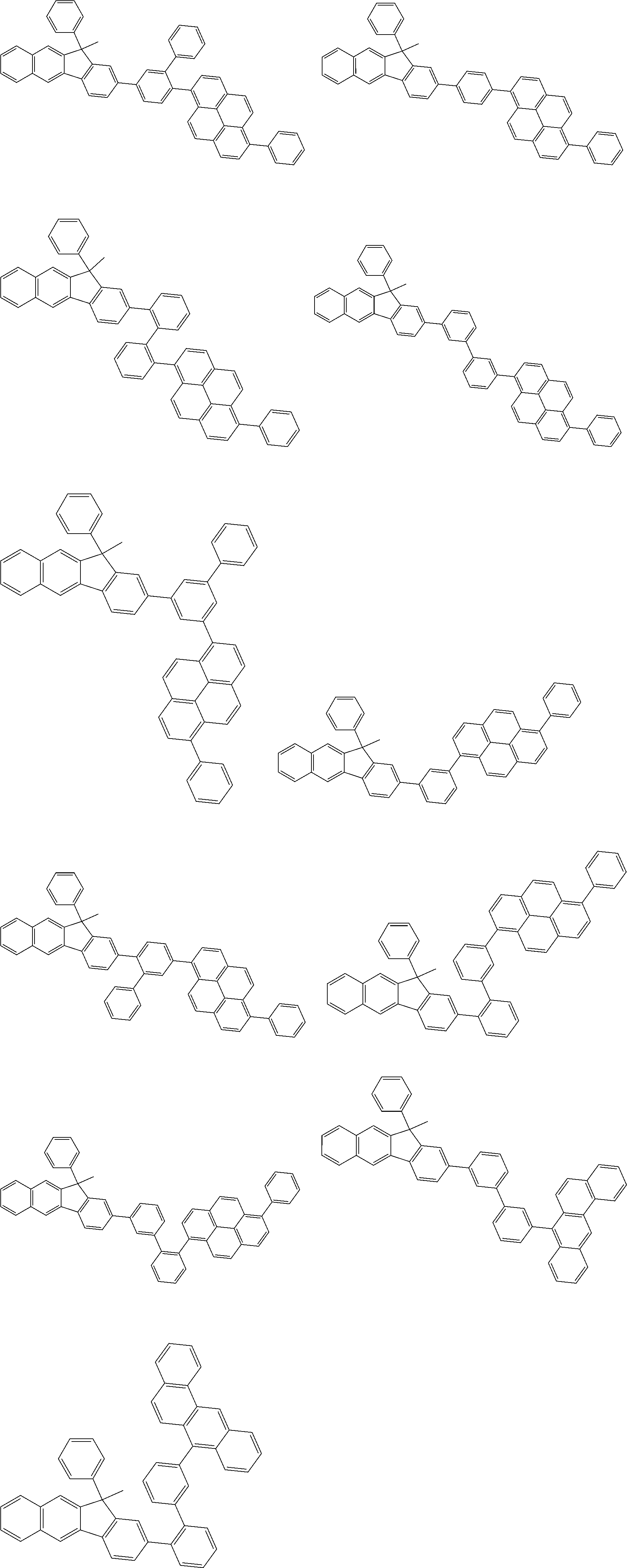

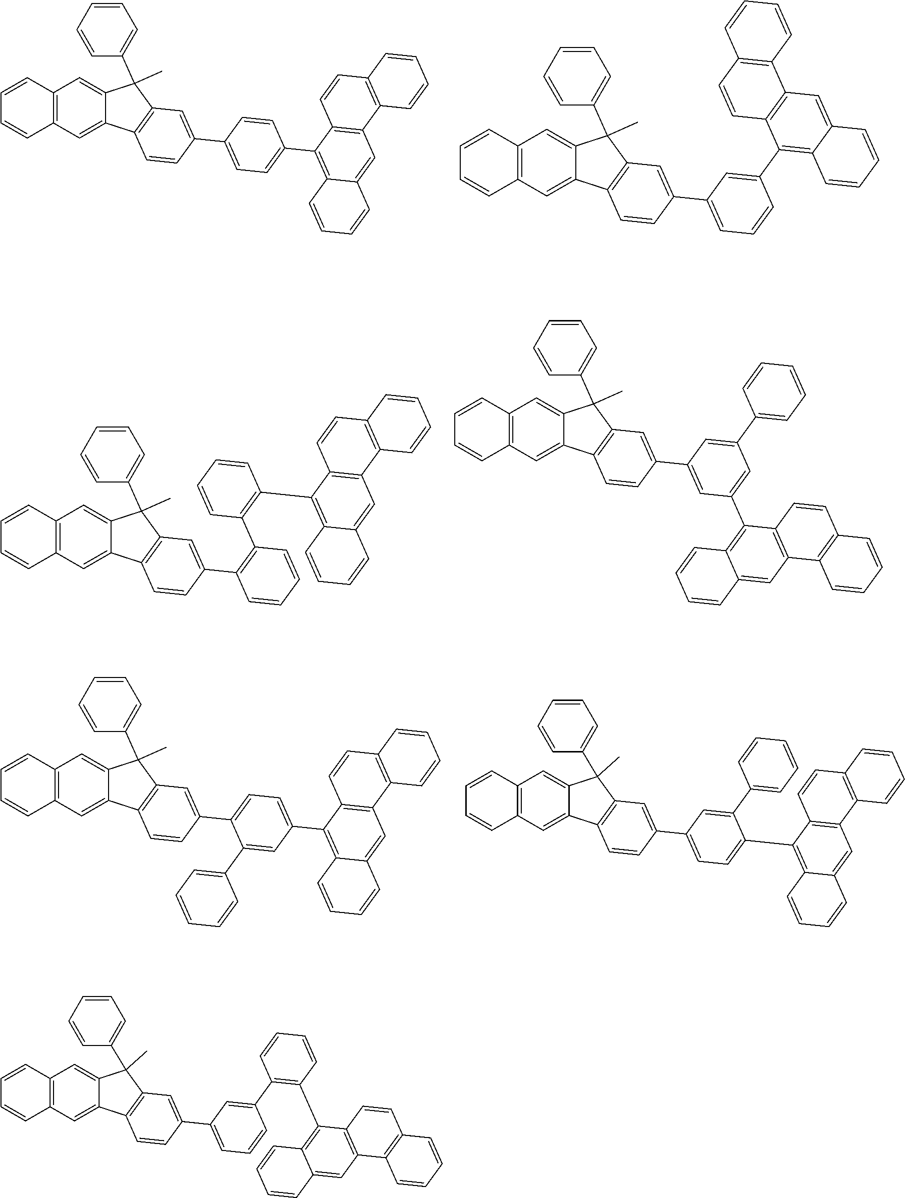

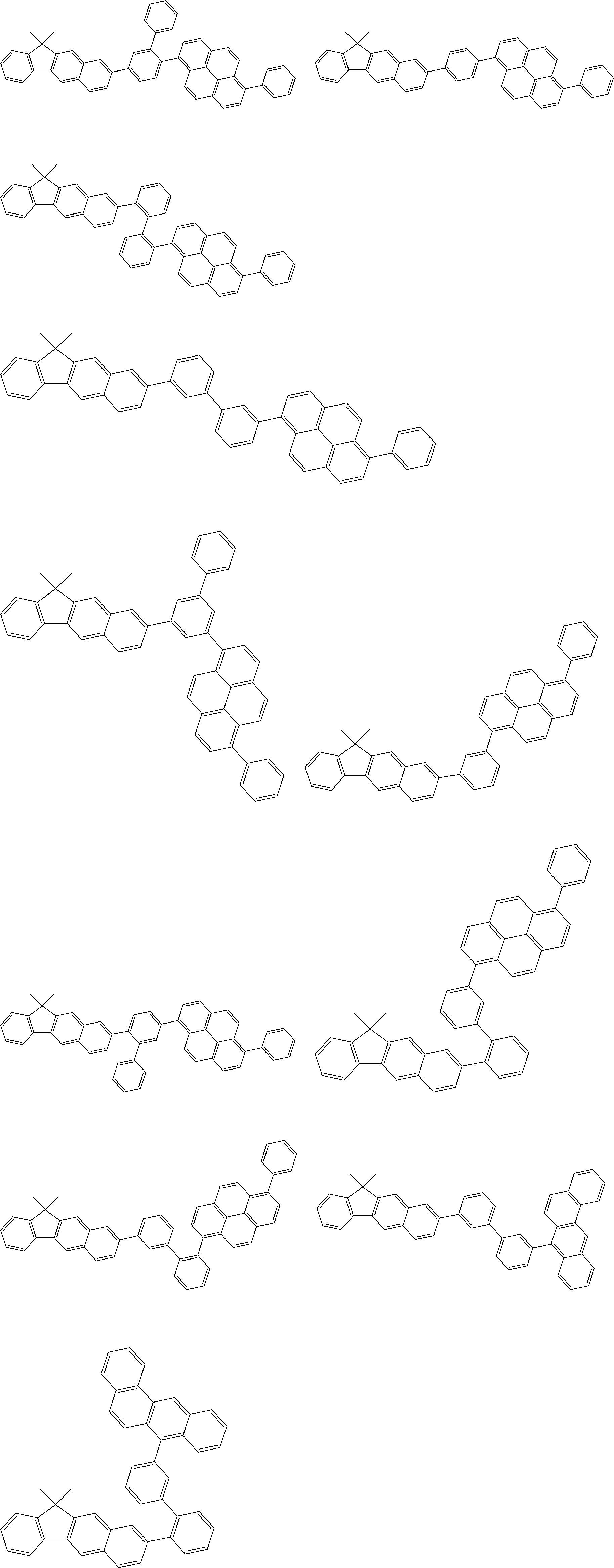

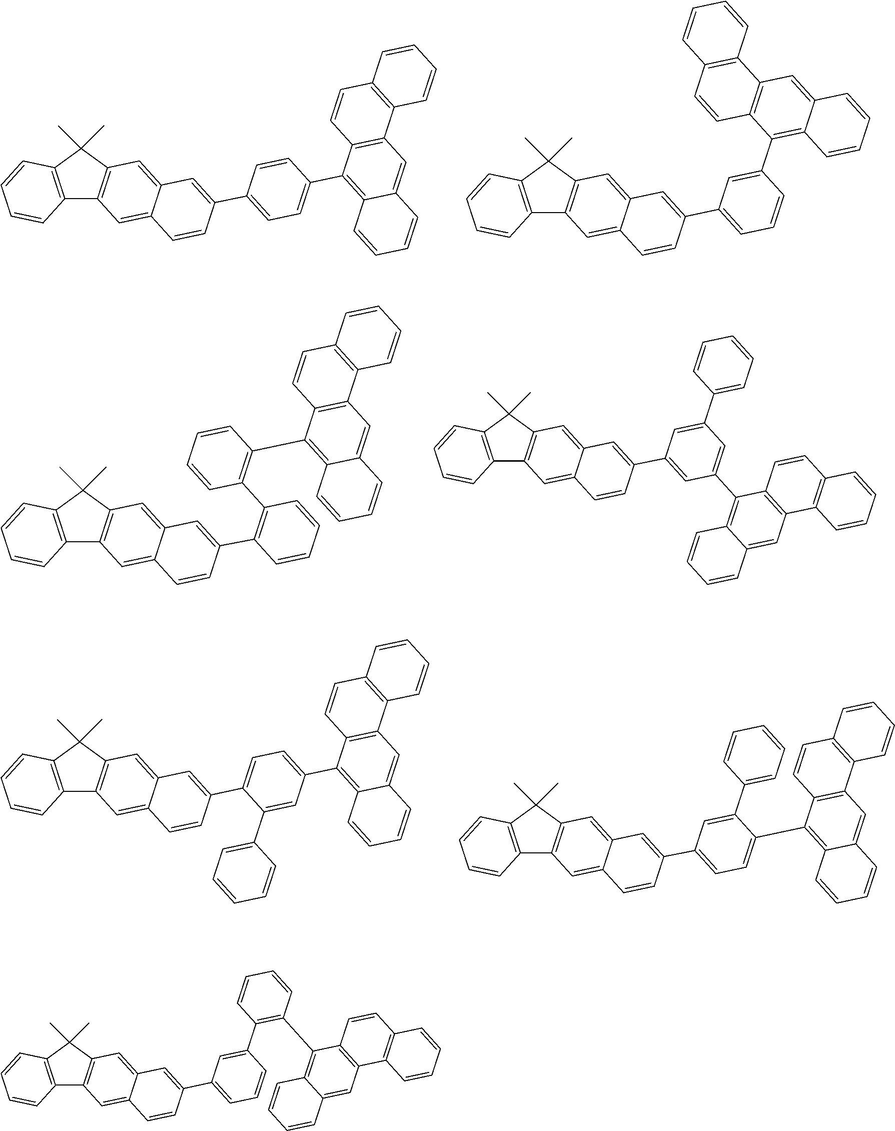

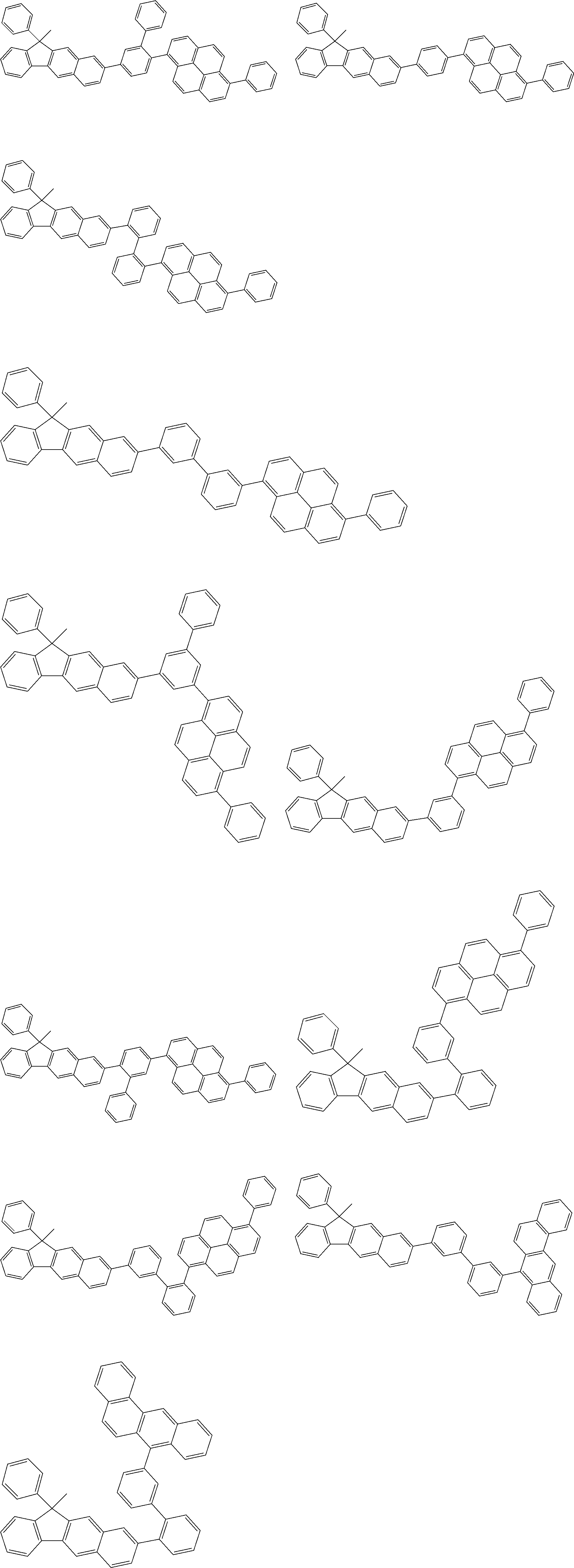

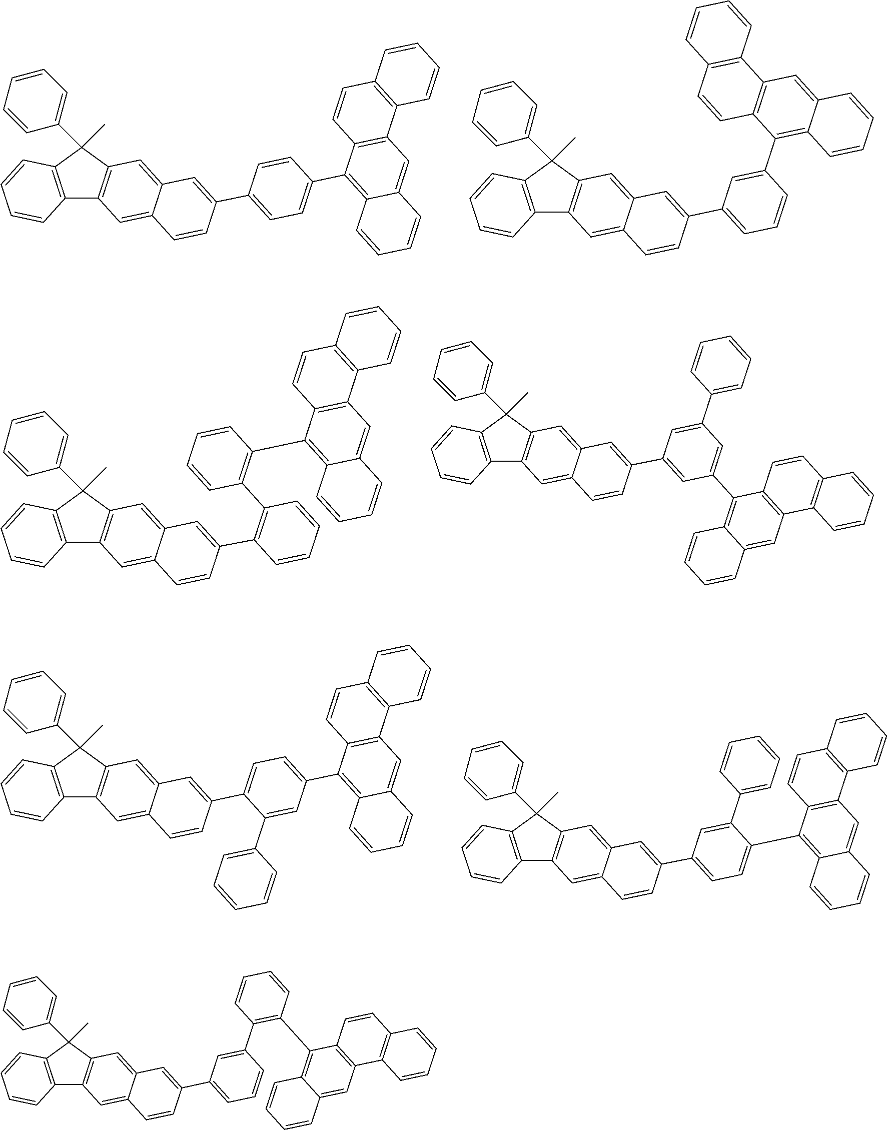

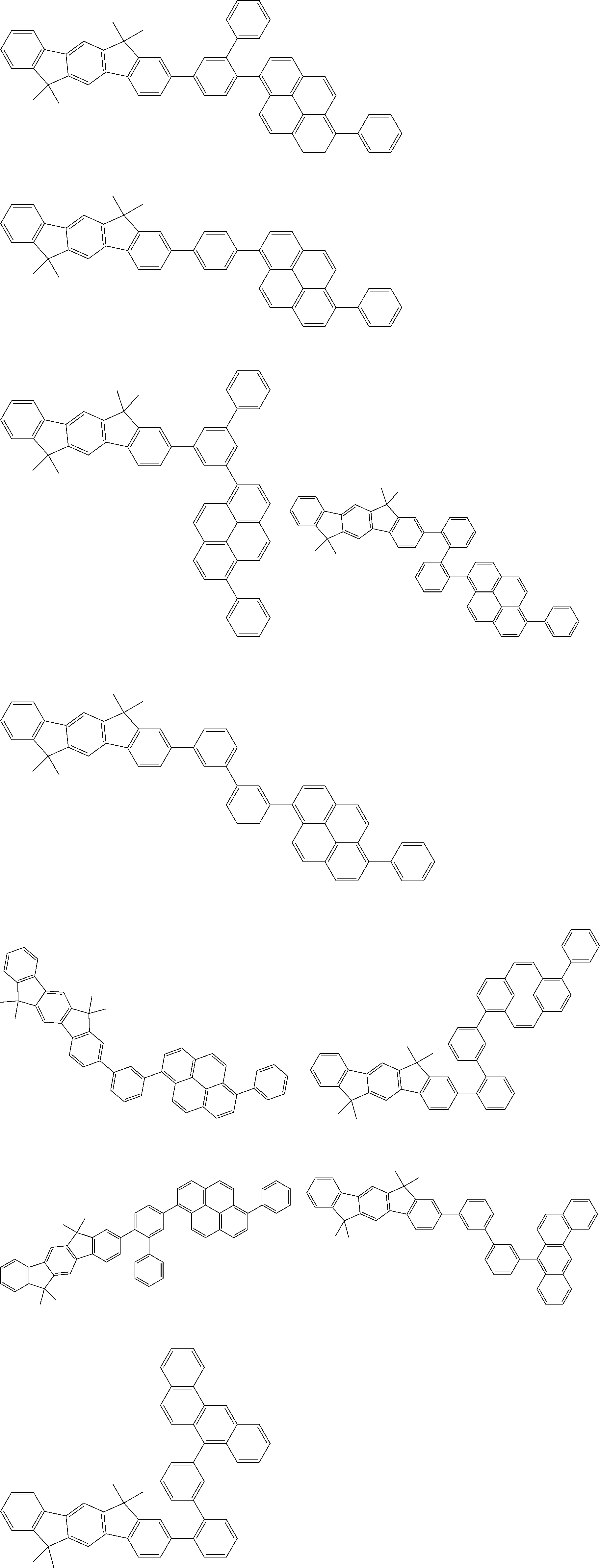

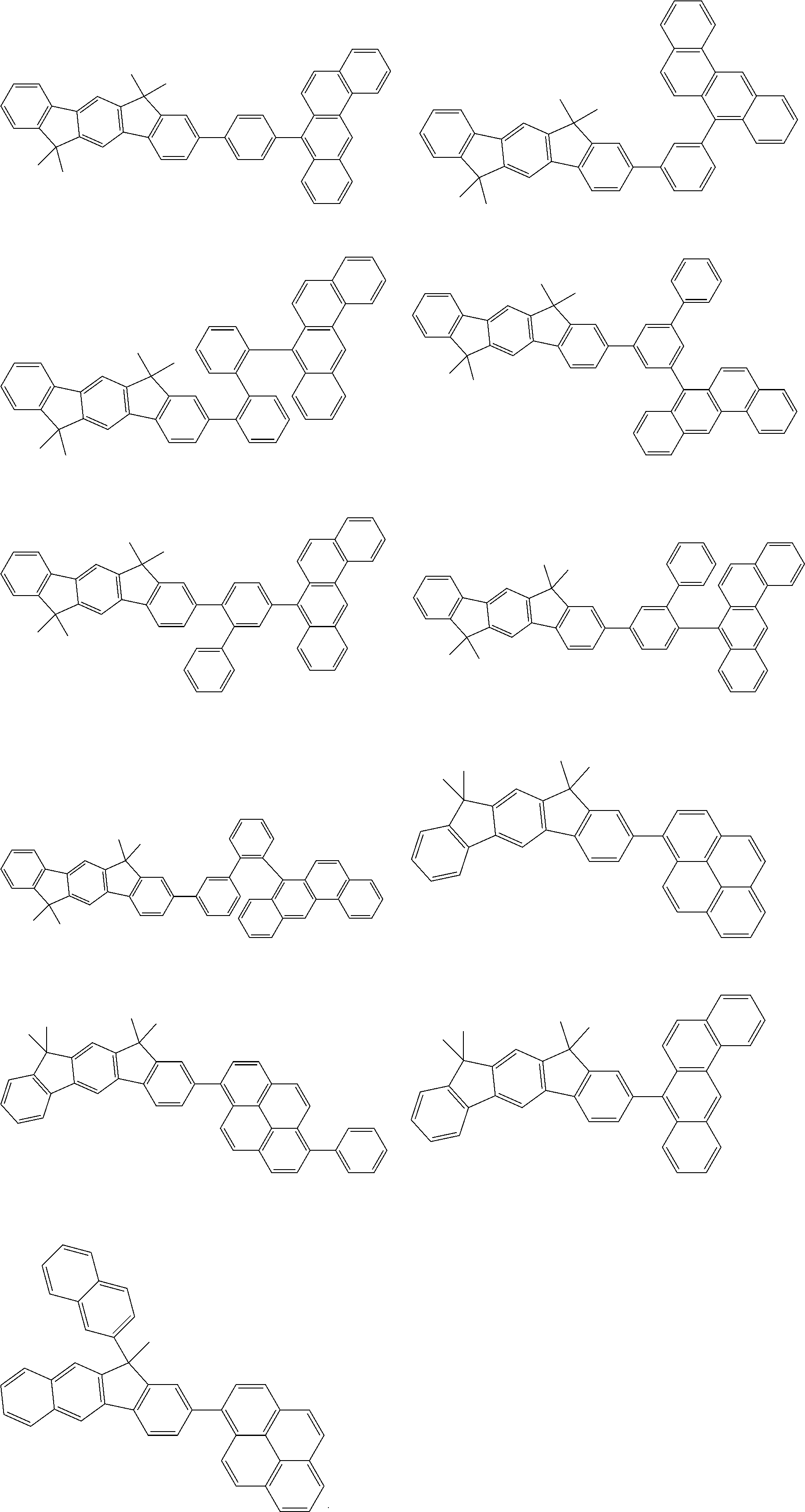

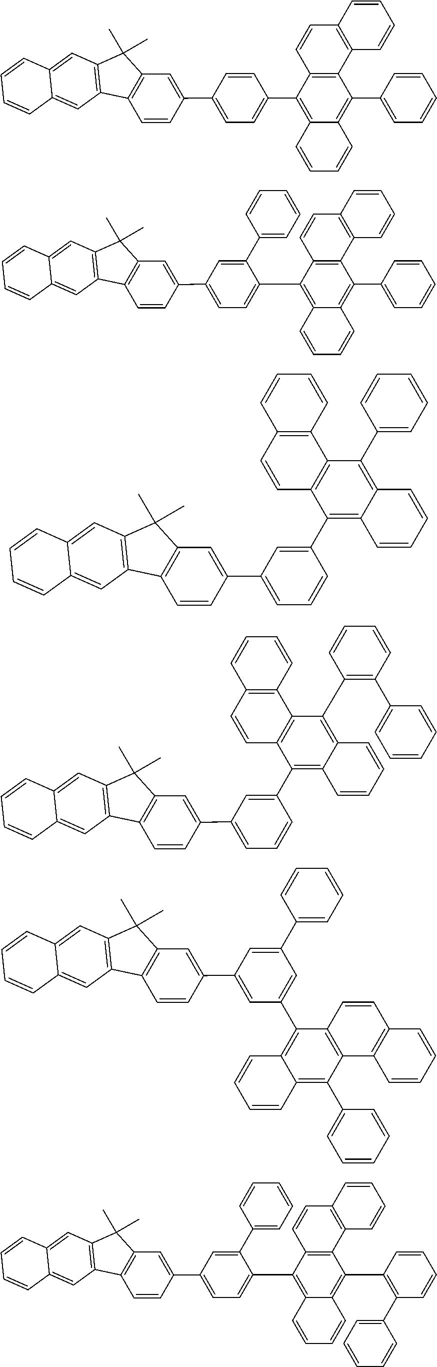

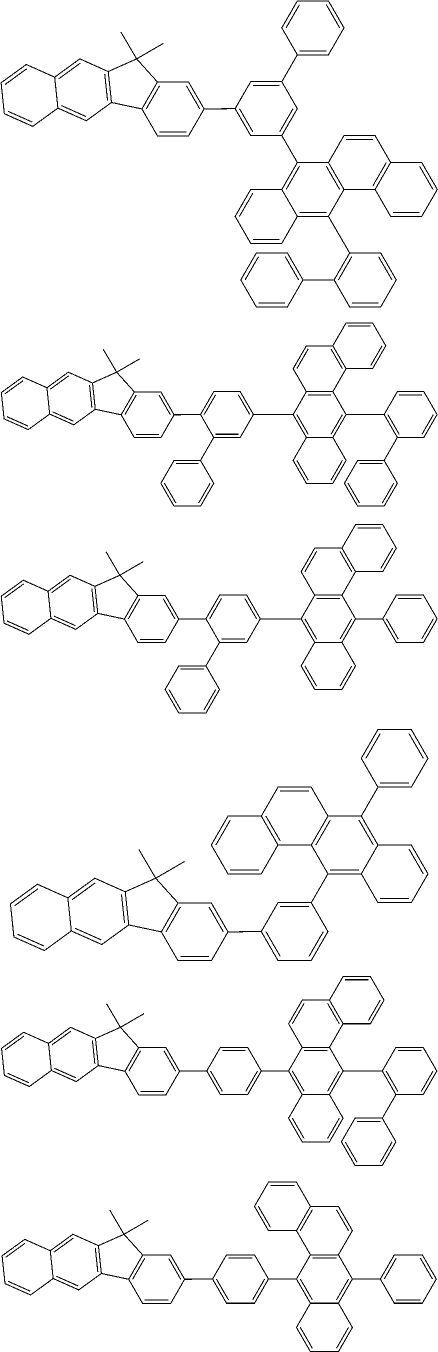

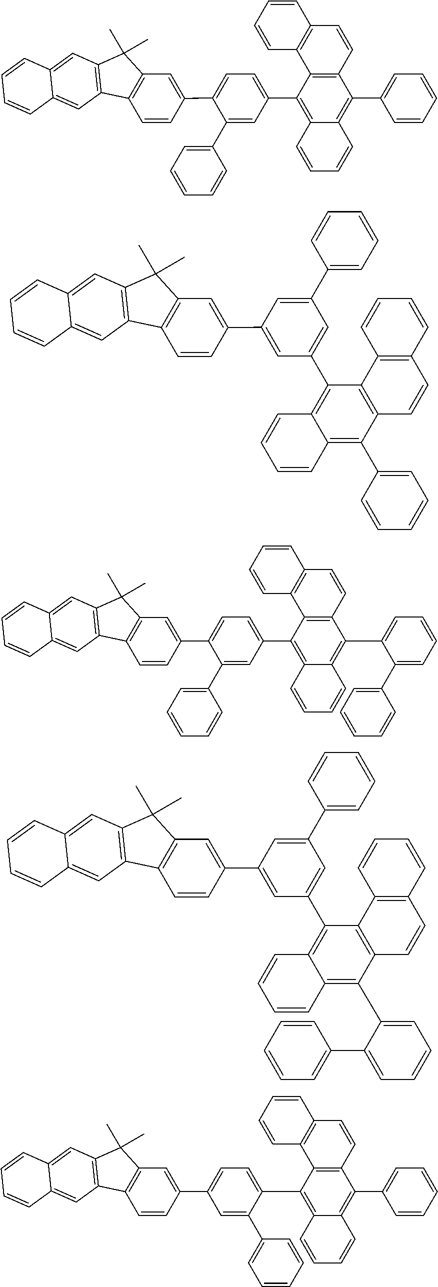

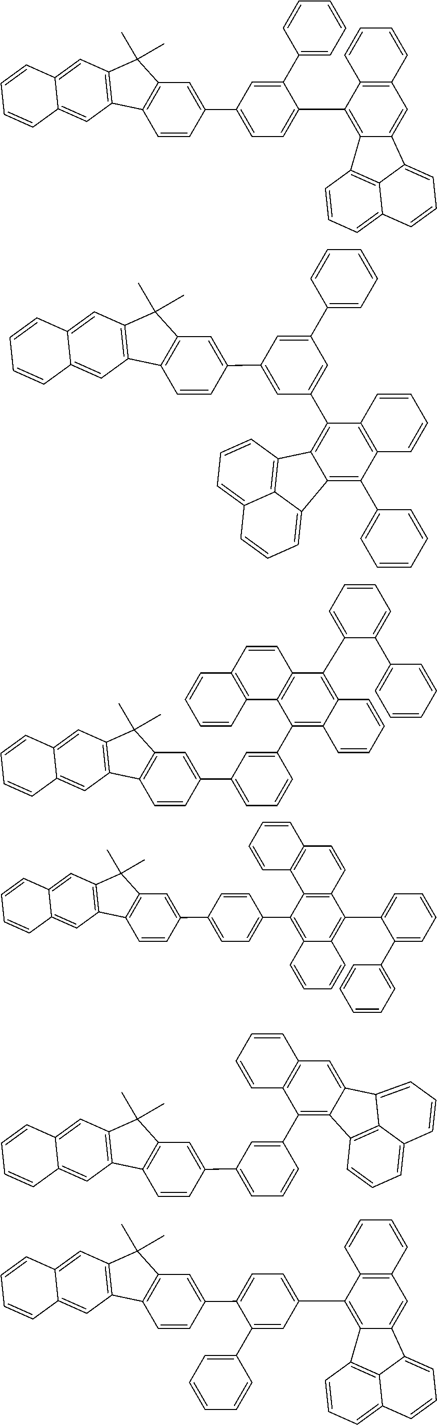

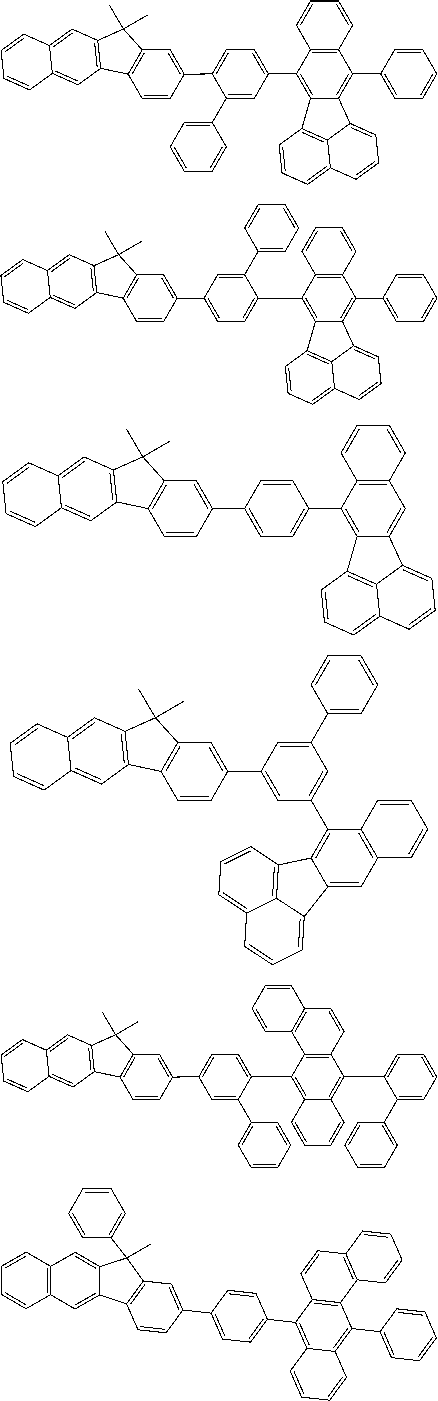

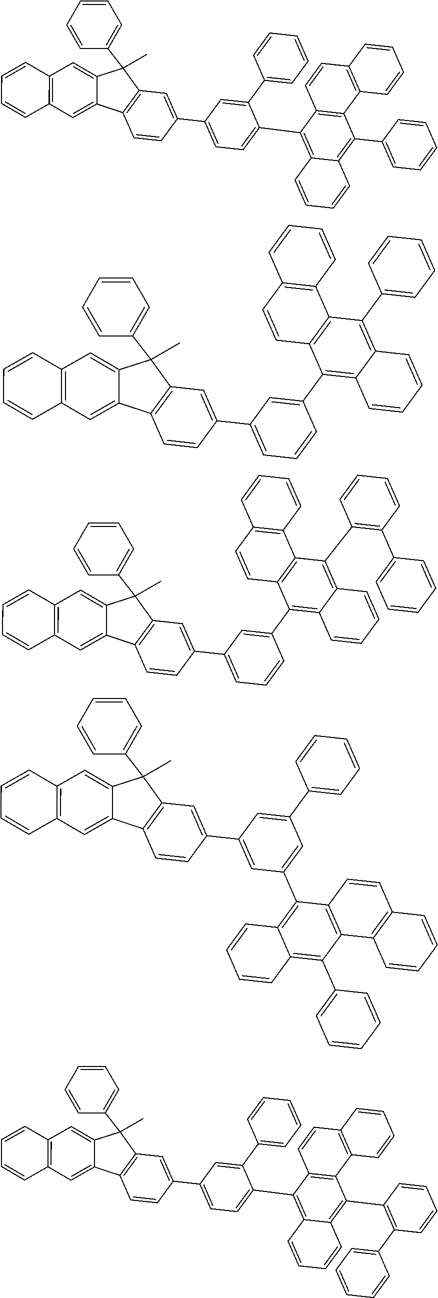

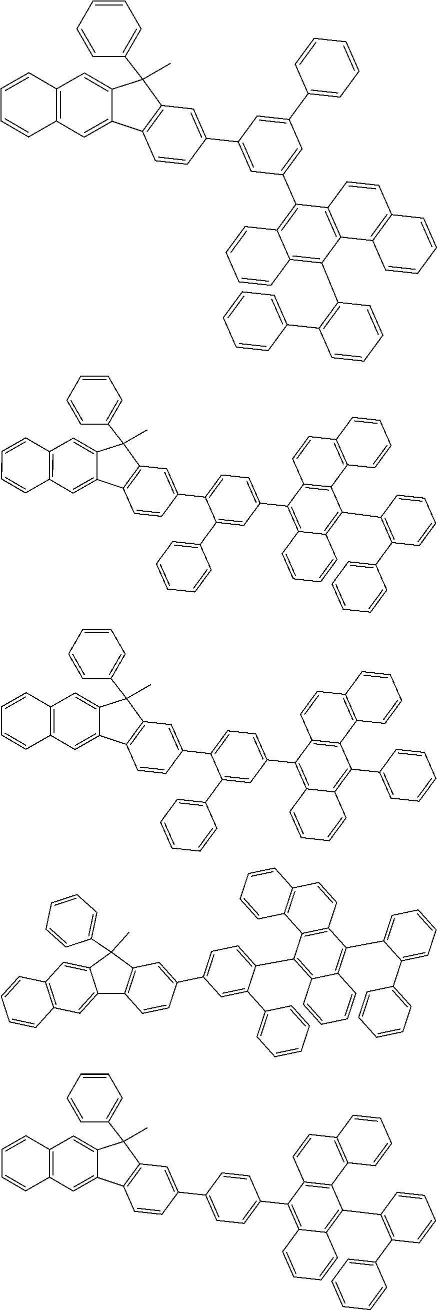

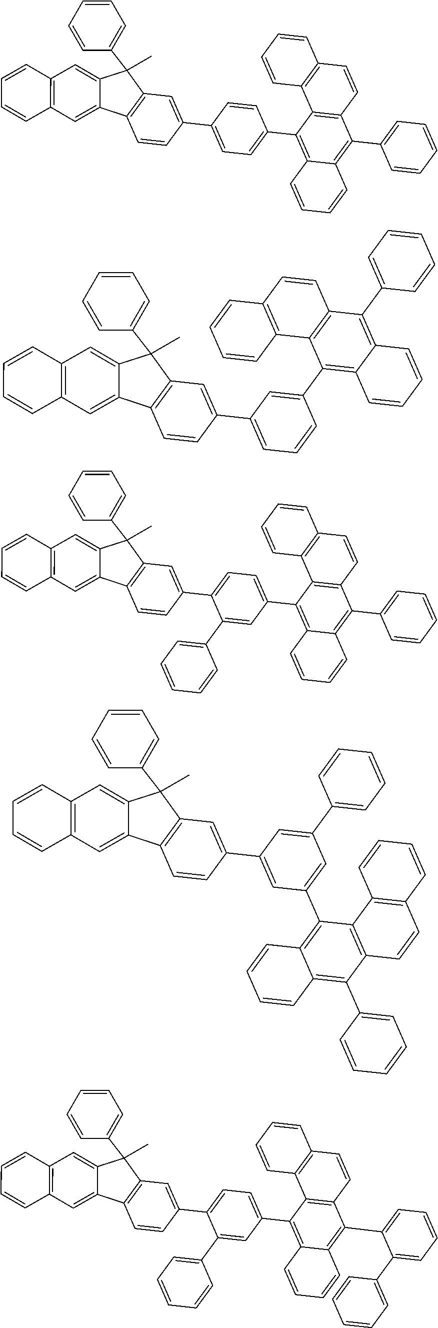

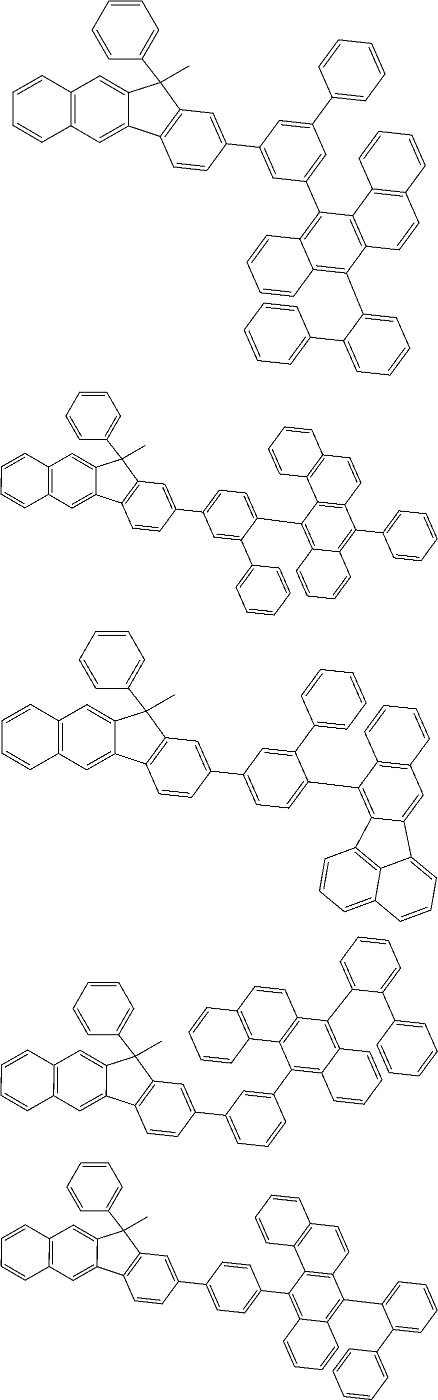

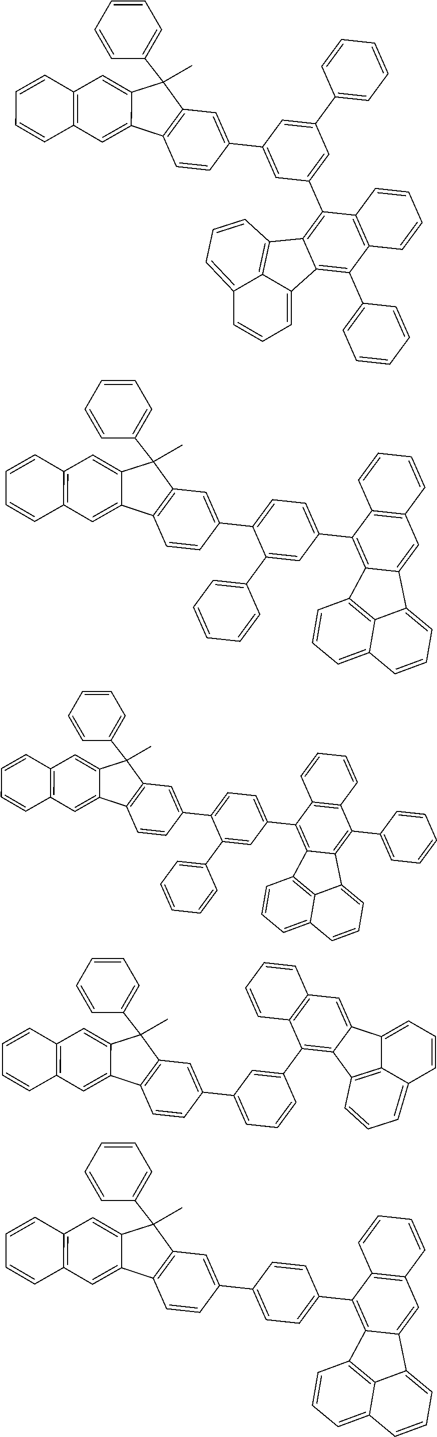

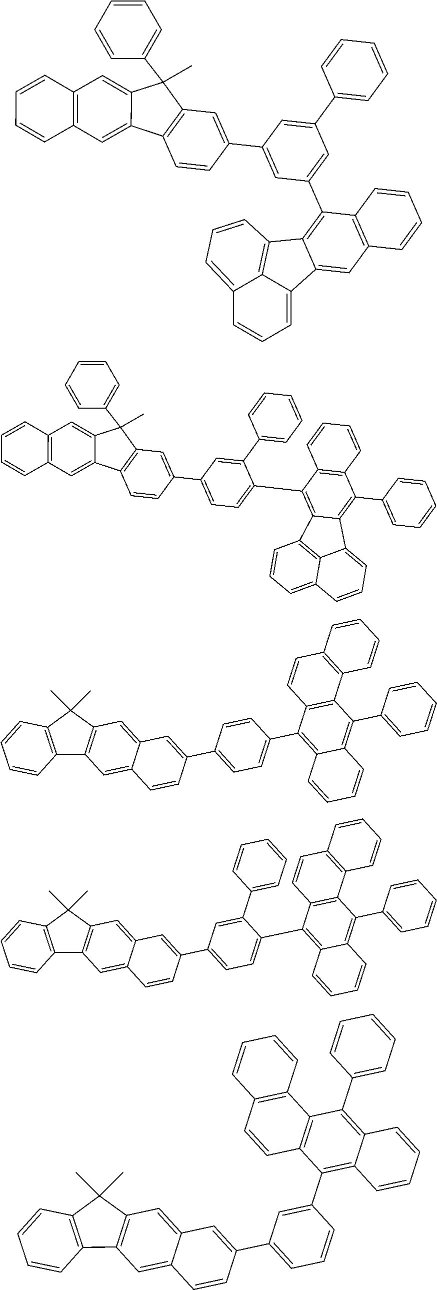

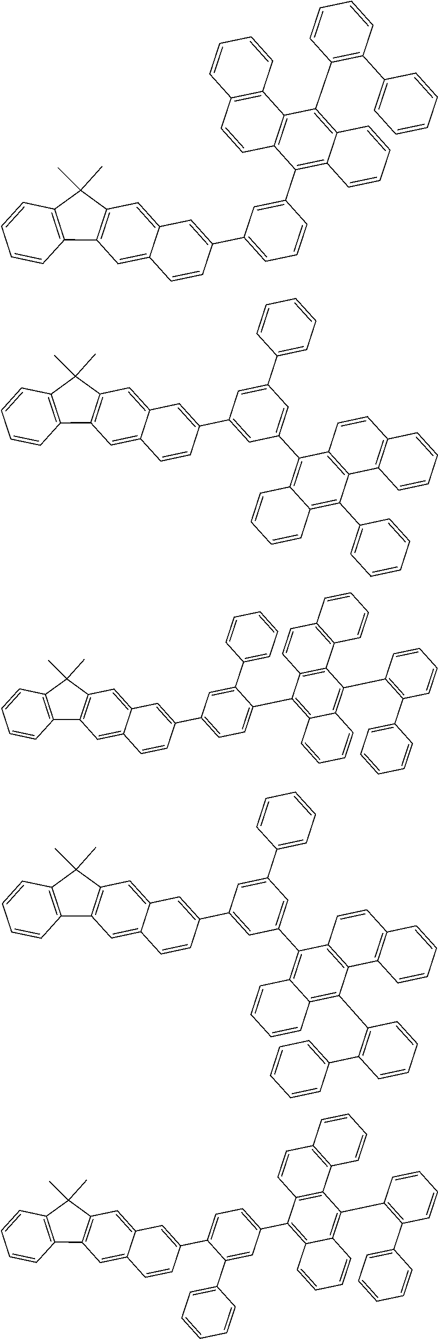

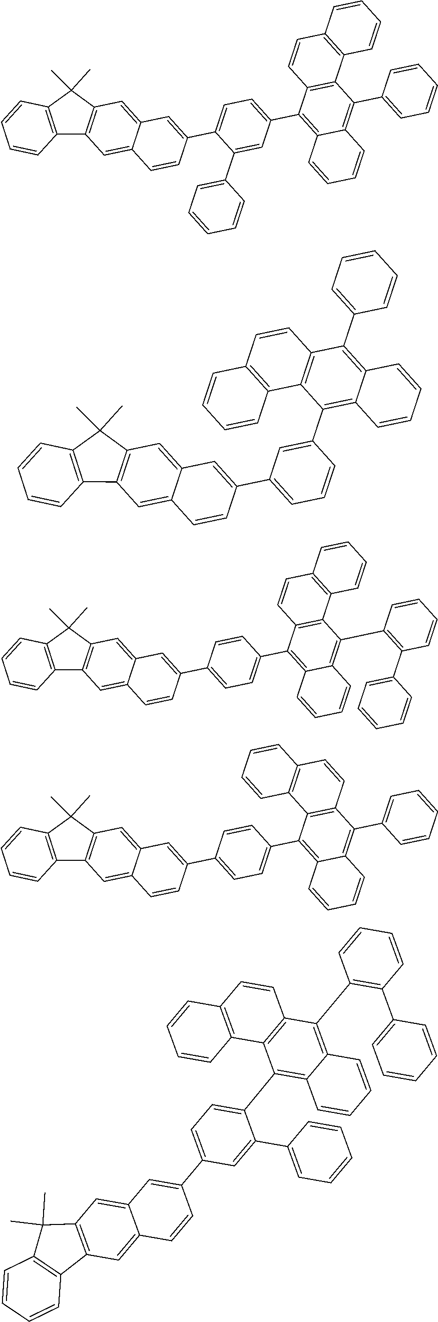

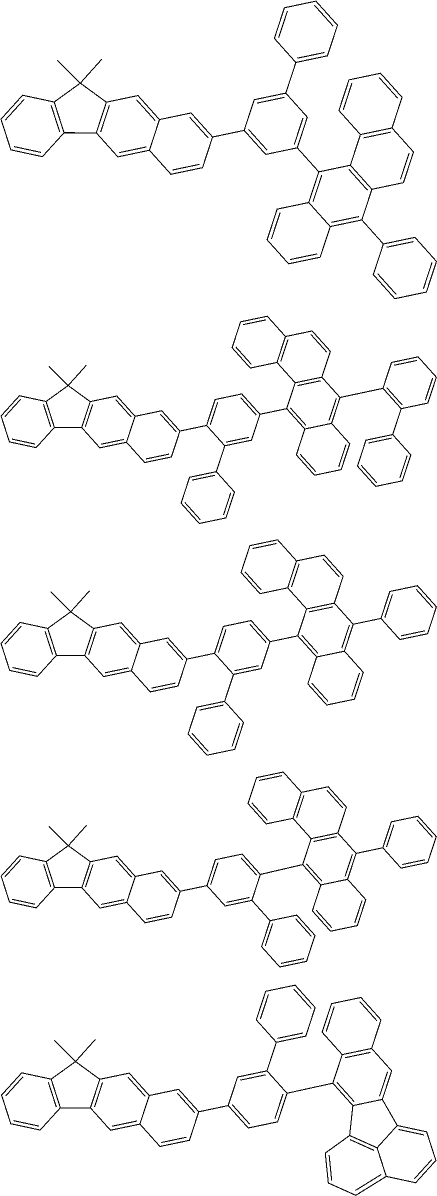

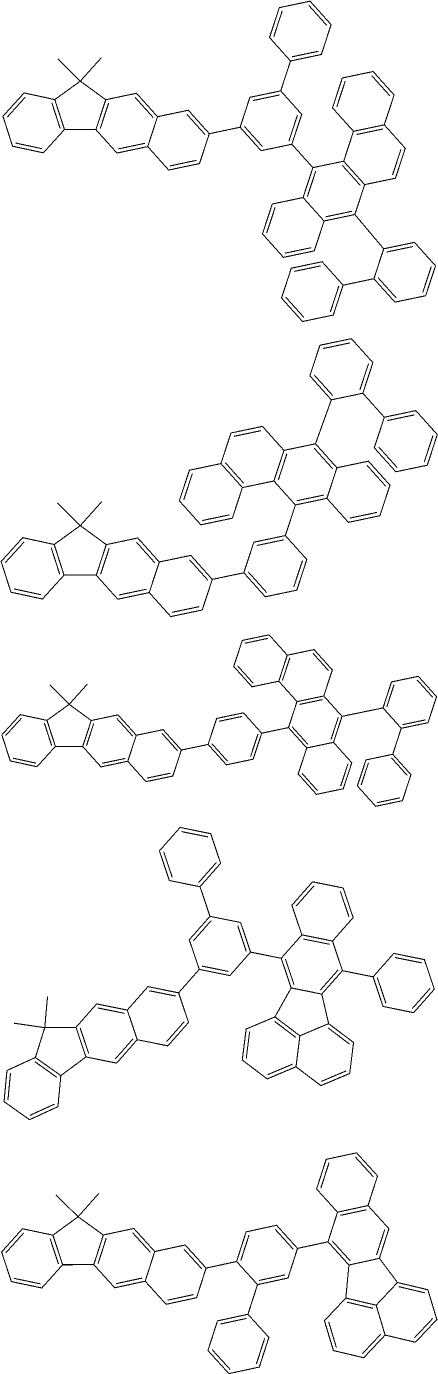

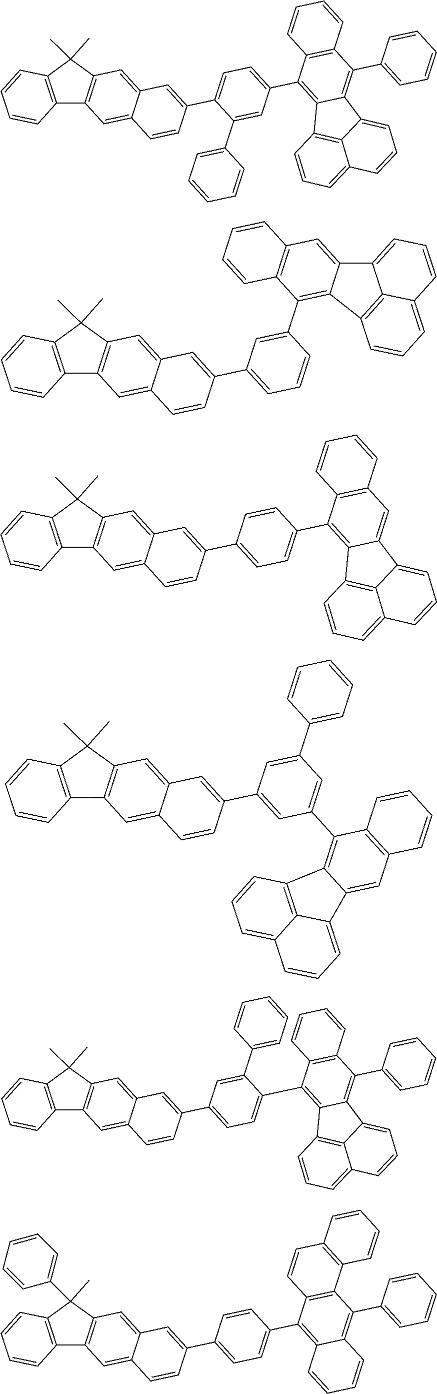

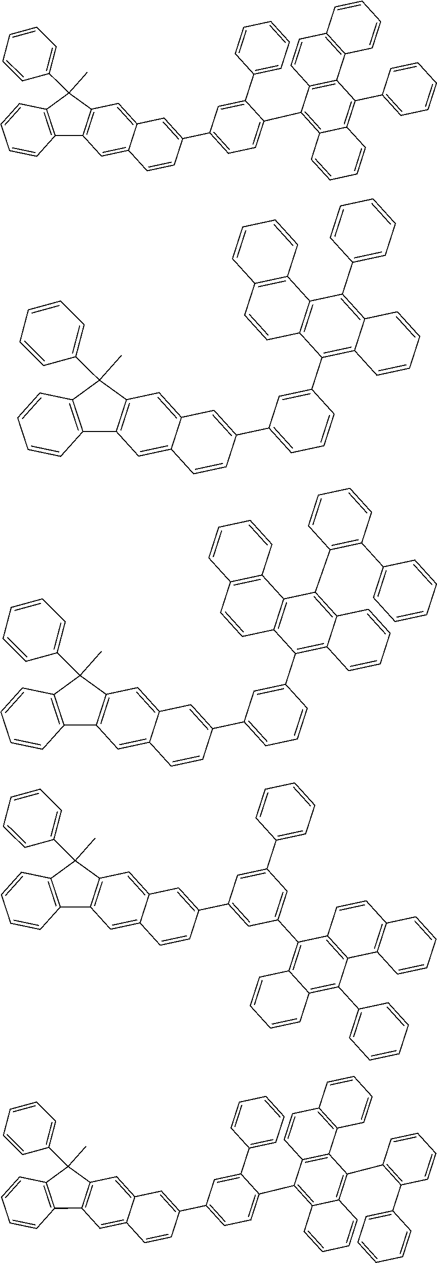

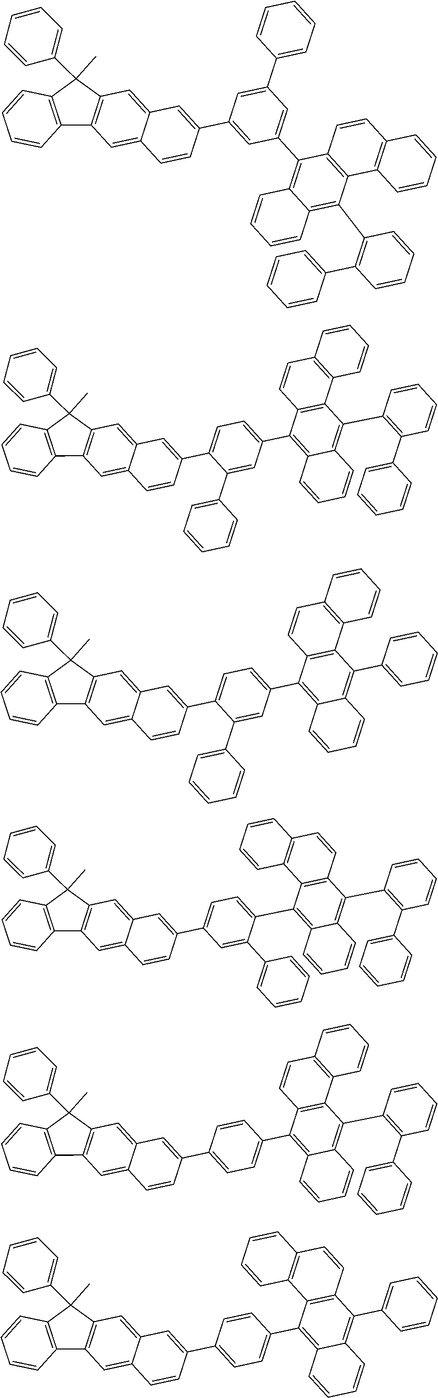

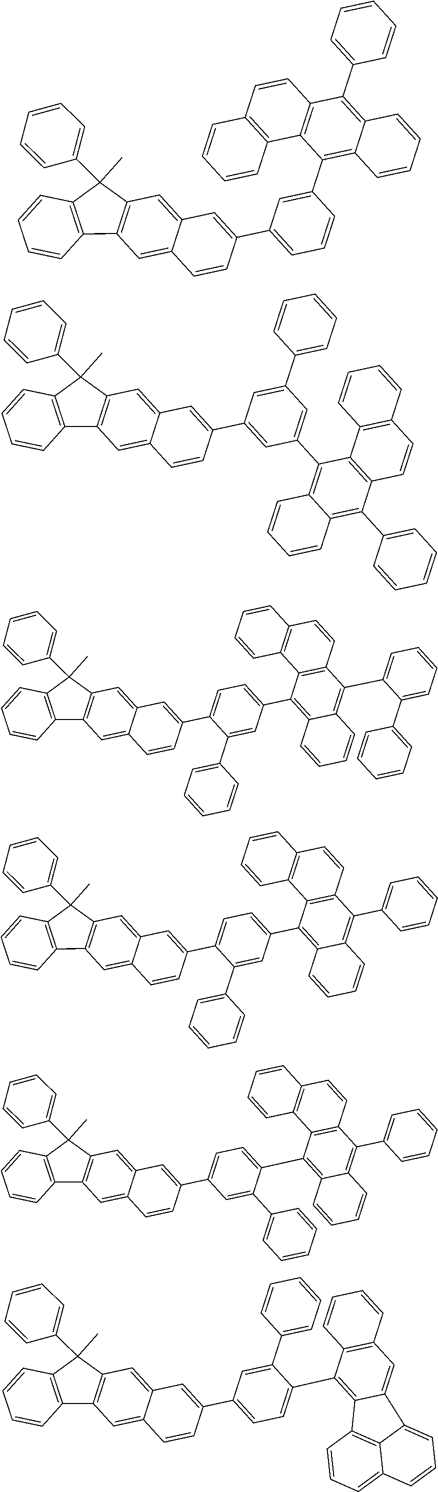

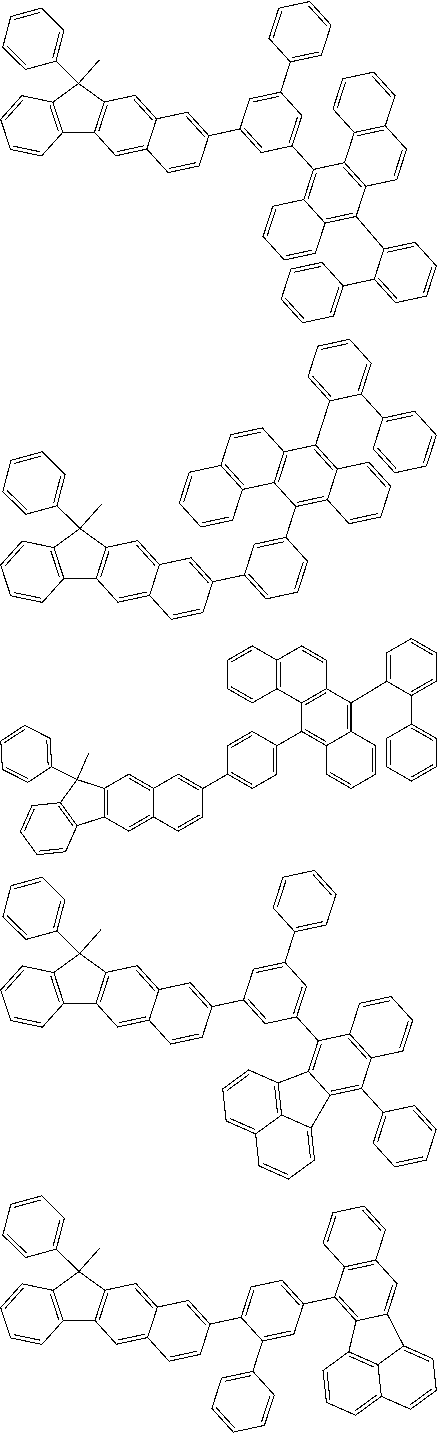

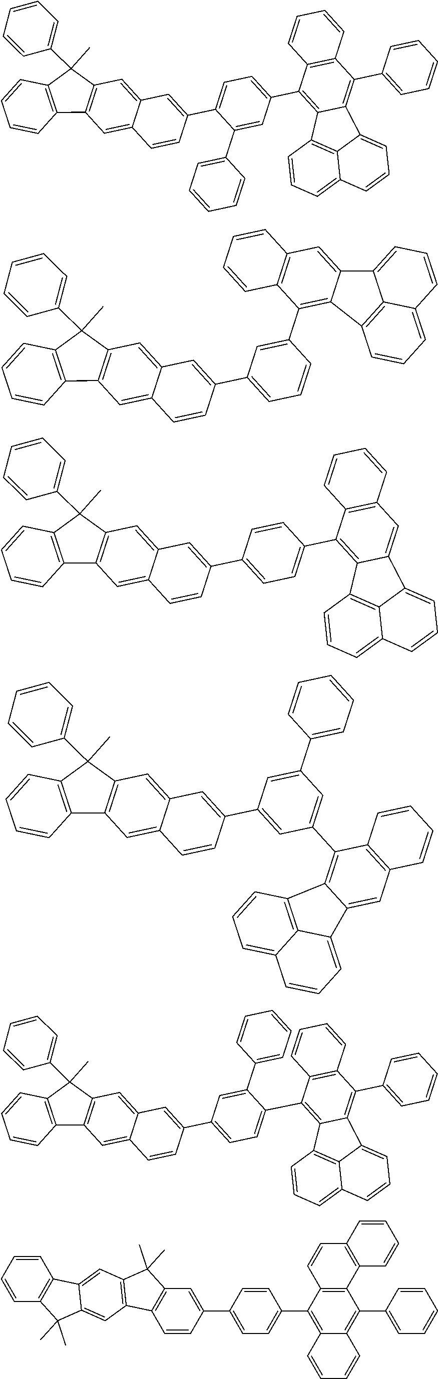

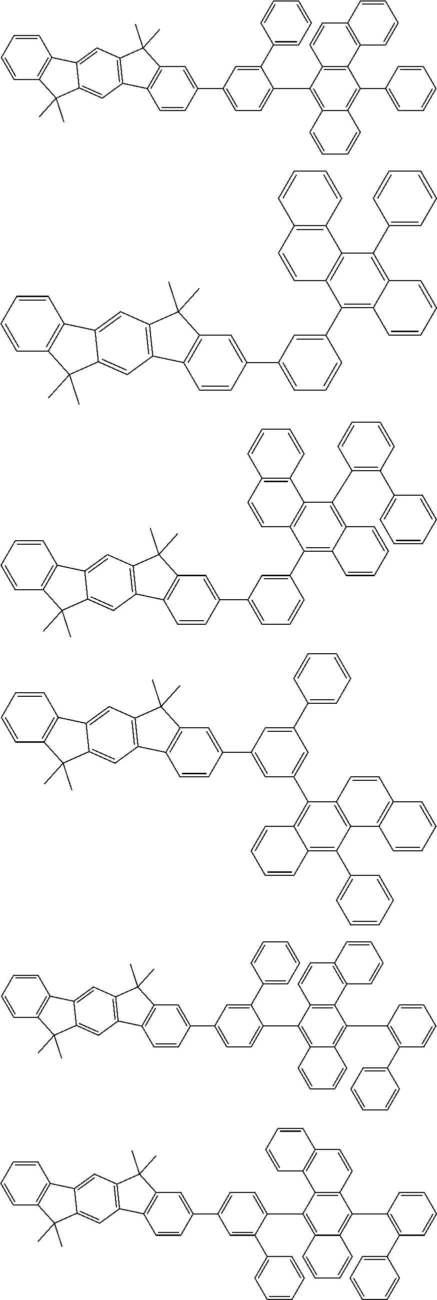

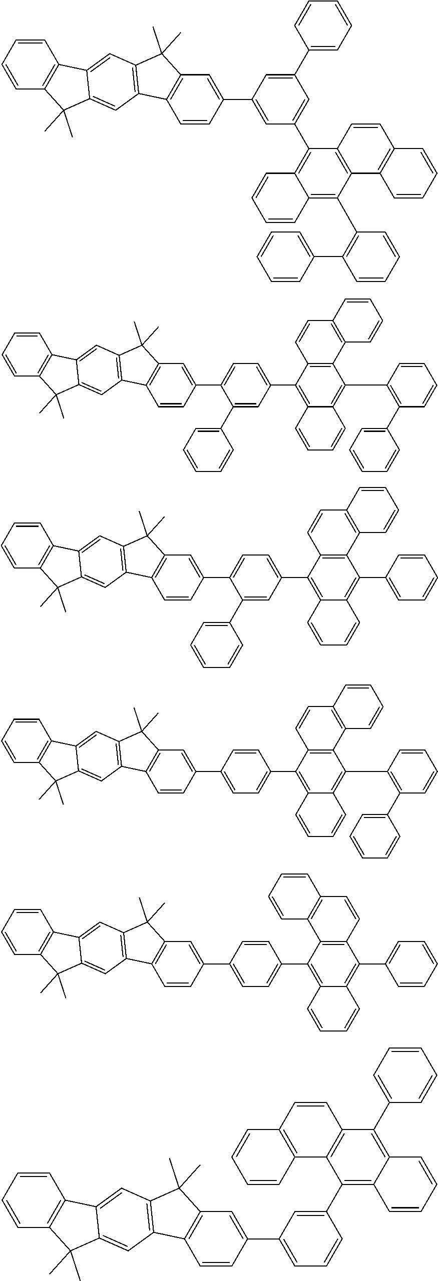

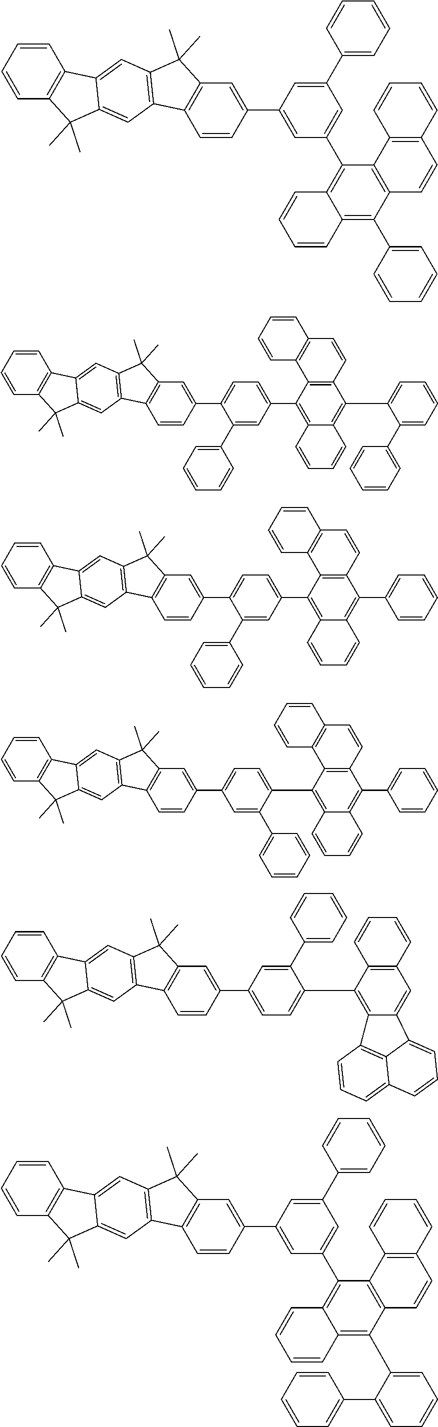

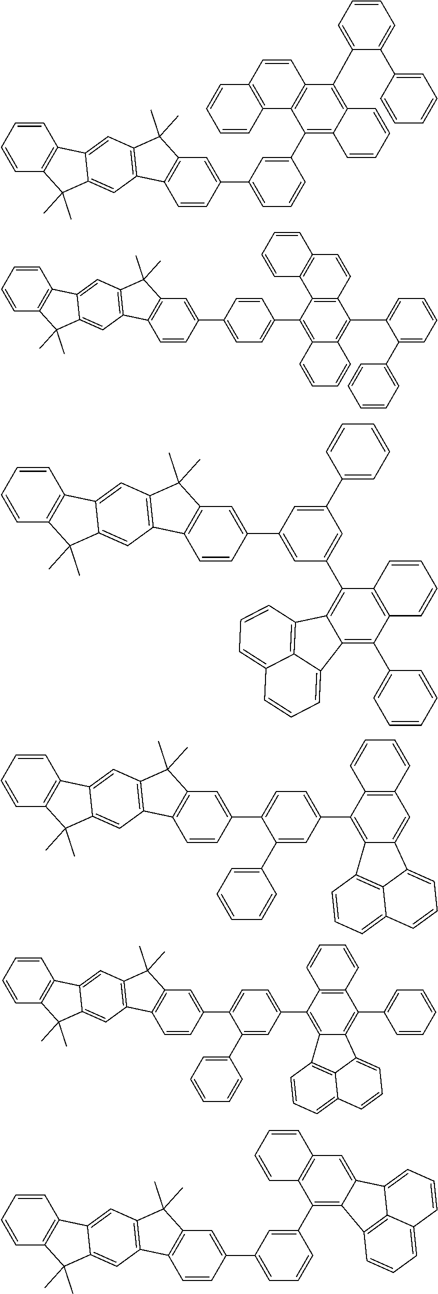

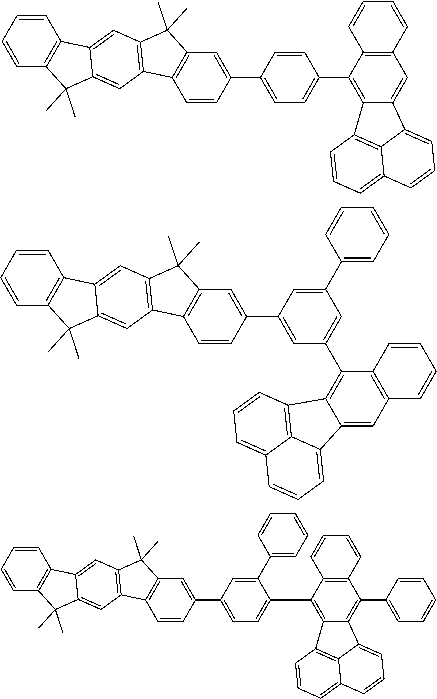

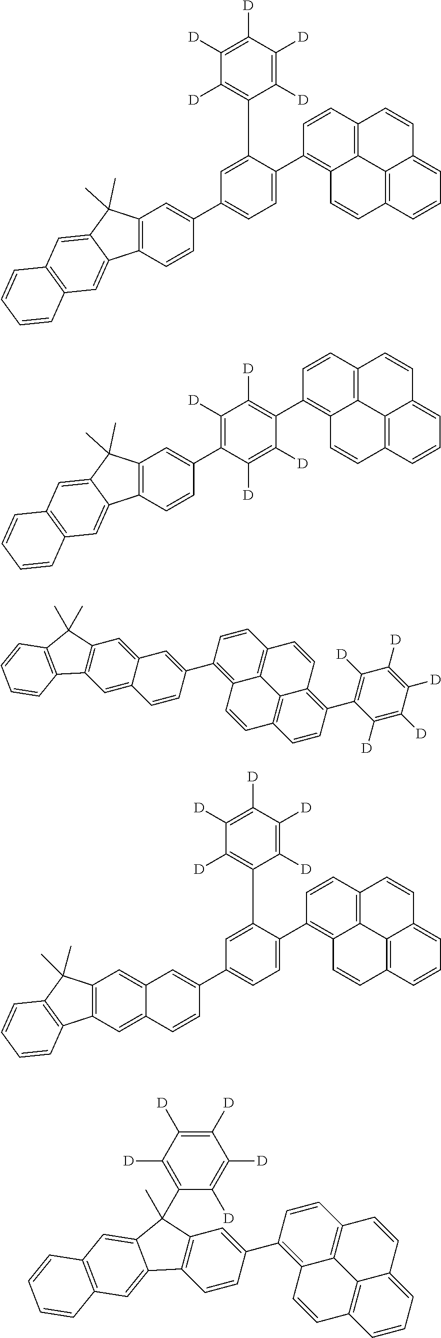

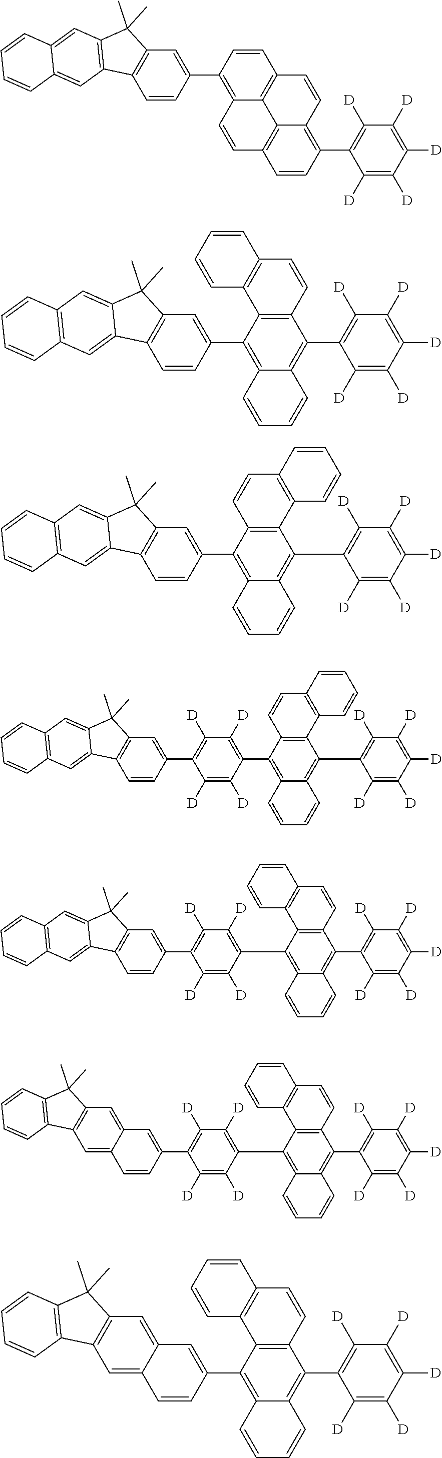

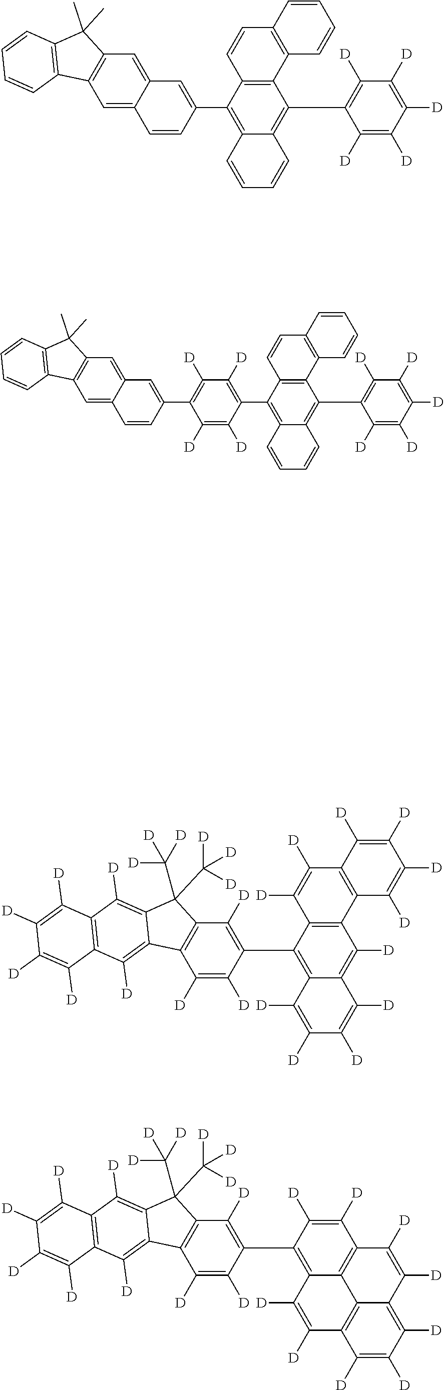

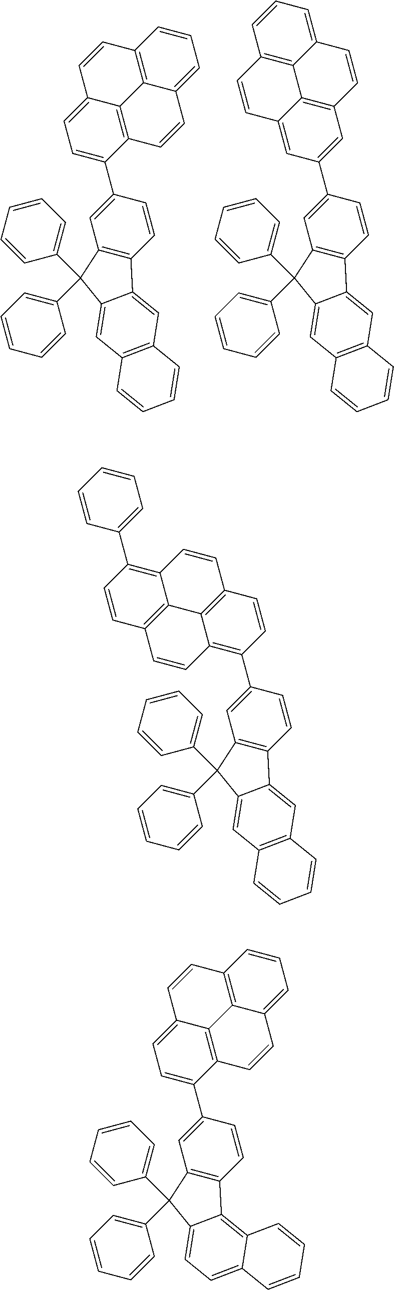

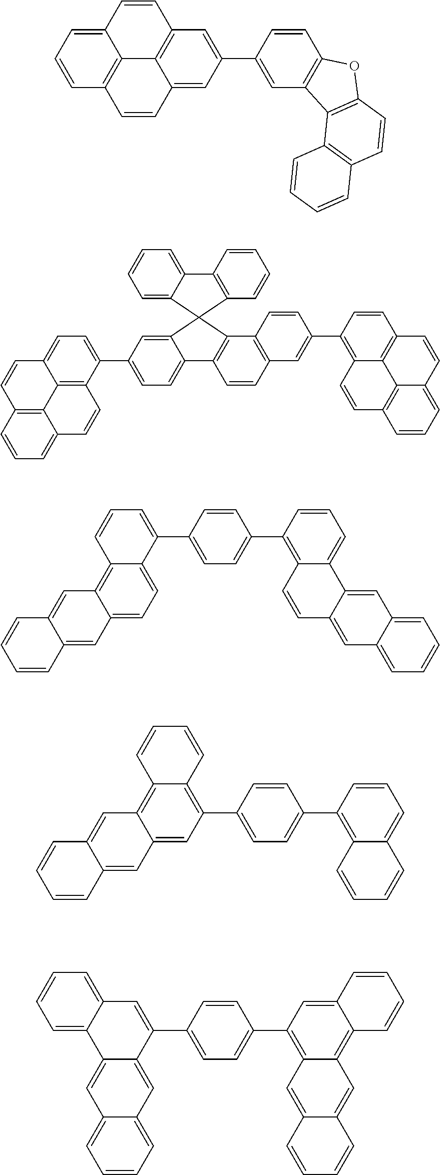

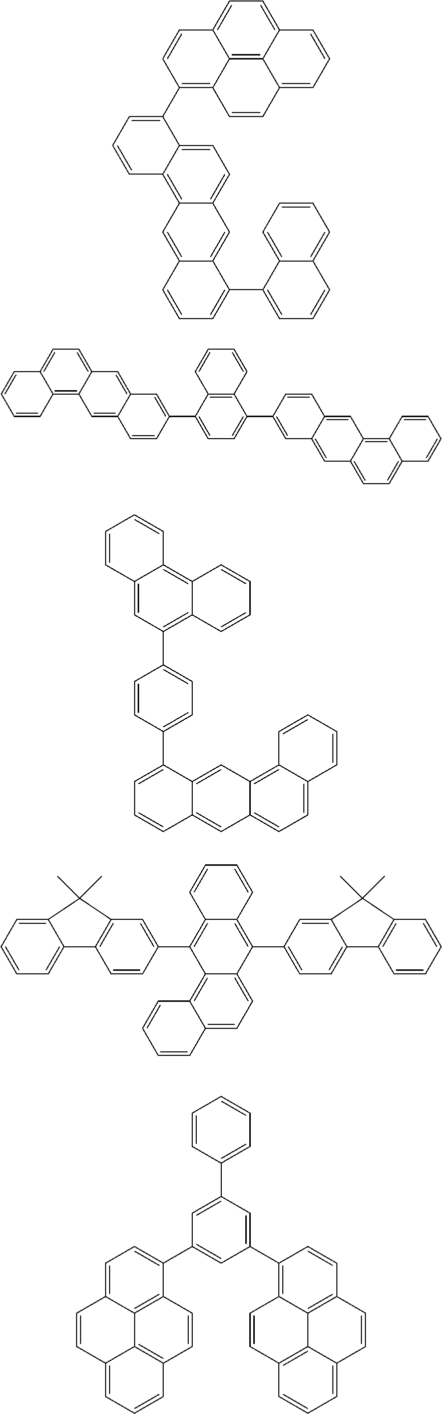

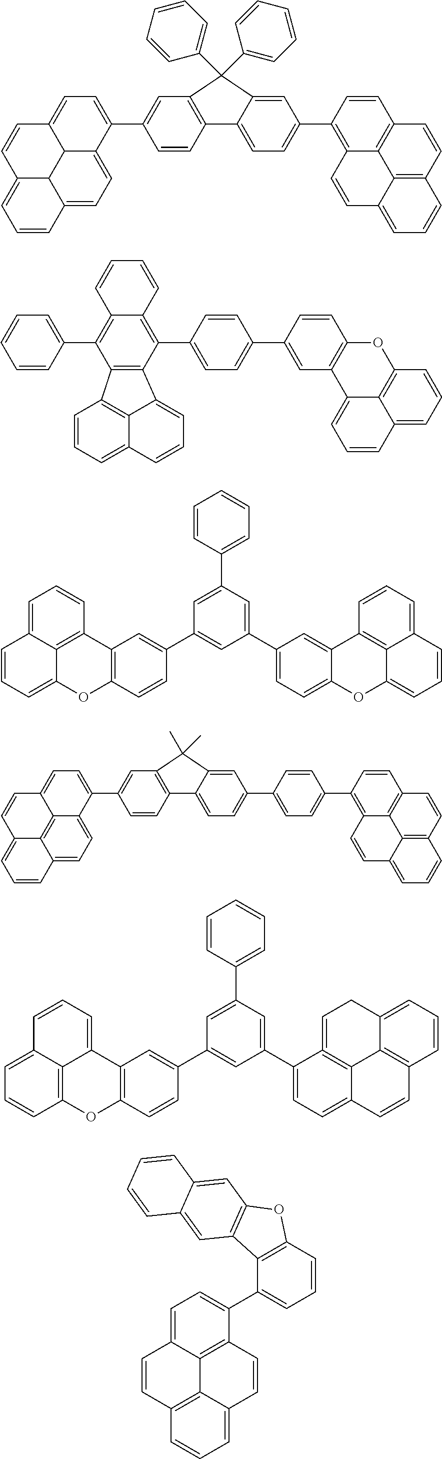

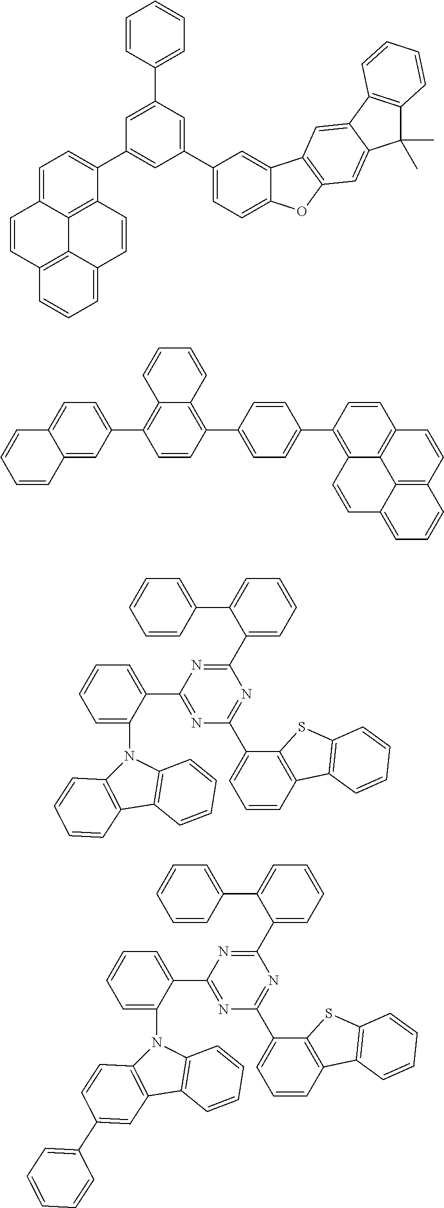

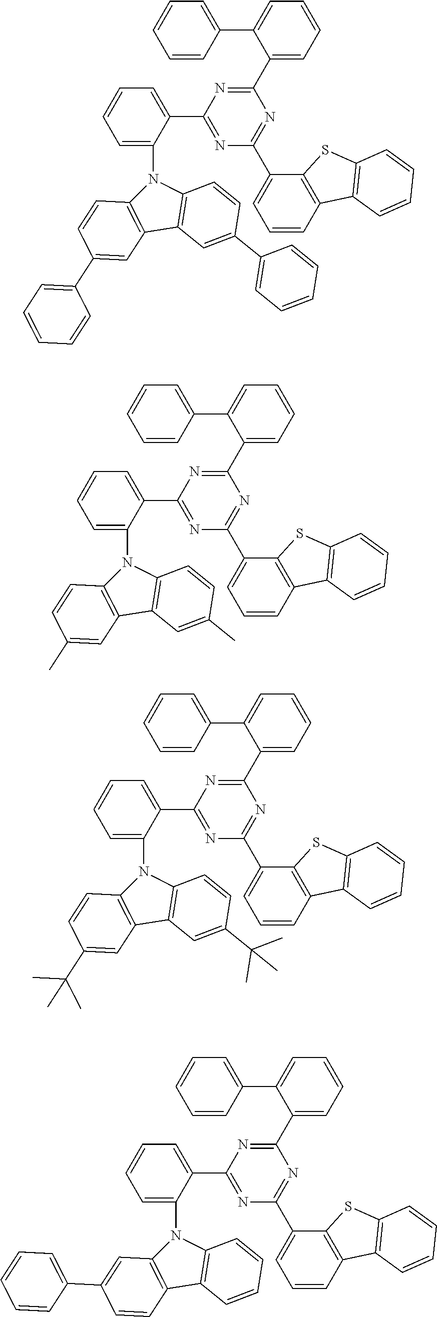 [SMILES: c1ccc(-c2ccc3c(c2)c2cc(-c4ccccc4)ccc2n3-c2ccccc2-c2nc(-c3ccccc3-c3ccccc3)nc(-c3cccc4c3sc3ccccc34)n2)cc1 ; Cc1ccc2c(c1)c1cc(C)ccc1n2-c1ccccc1-c1nc(-c2ccccc2-c2ccccc2)nc(-c2cccc3c2sc2ccccc23)n1 ; CC(C)(C)c1ccc2c(c1)c1cc(C(C)(C)C)ccc1n2-c1ccccc1-c1nc(-c2ccccc2-c2ccccc2)nc(-c2cccc3c2sc2ccccc23)n1 ; c1ccc(-c2ccc3c4ccccc4n(-c4ccccc4-c4nc(-c5ccccc5-c5ccccc5)nc(-c5cccc6c5sc5ccccc56)n4)c3c2)cc1]

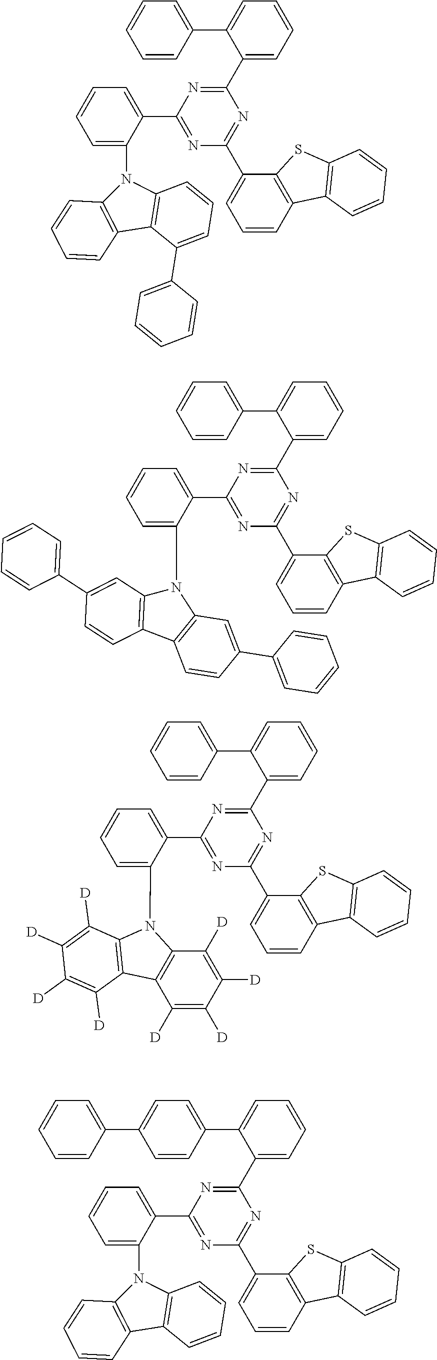

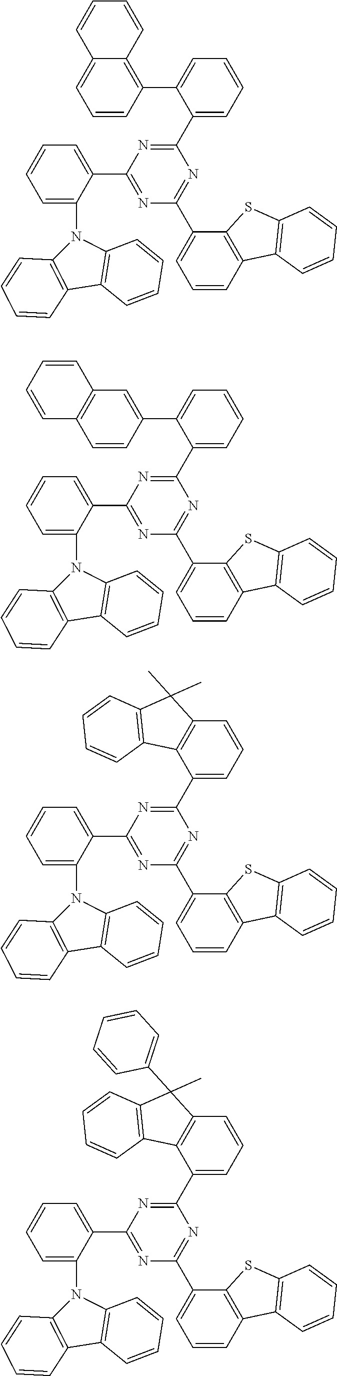

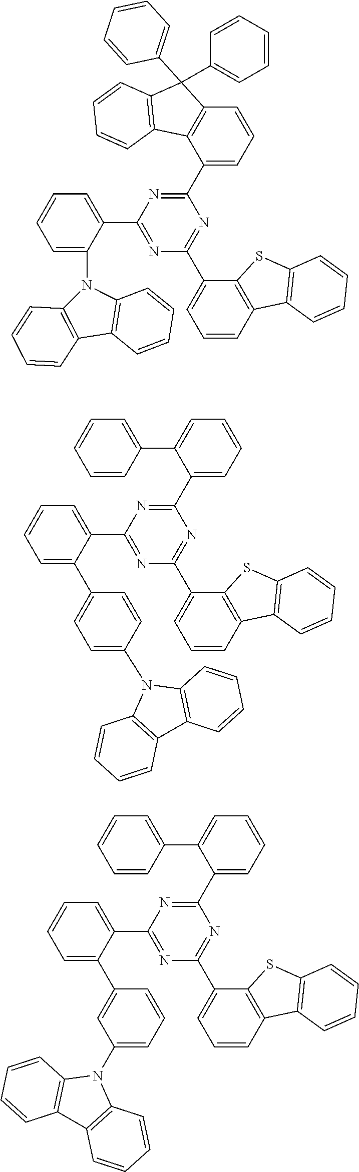

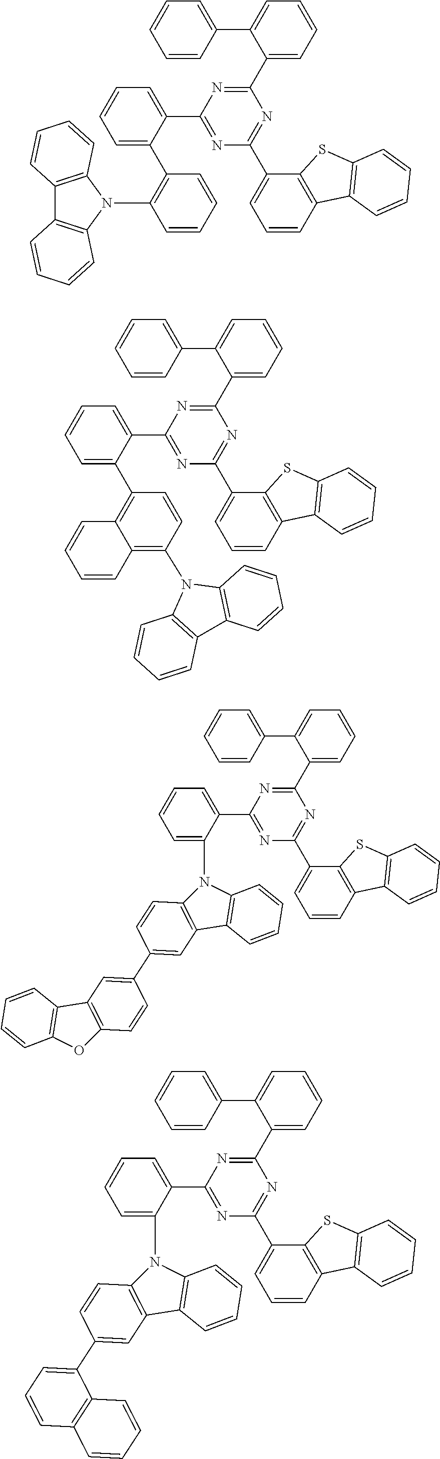 [SMILES: c1ccc(-c2ccccc2-c2nc(-c3ccccc3-c3ccccc3-n3c4ccccc4c4ccccc43)nc(-c3cccc4c3sc3ccccc34)n2)cc1 ; c1ccc(-c2ccccc2-c2nc(-c3ccccc3-c3ccc(-n4c5ccccc5c5ccccc54)c4ccccc34)nc(-c3cccc4c3sc3ccccc34)n2)cc1 ; c1ccc(-c2ccccc2-c2nc(-c3ccccc3-n3c4ccccc4c4cc(-c5ccc6oc7ccccc7c6c5)ccc43)nc(-c3cccc4c3sc3ccccc34)n2)cc1 ; c1ccc(-c2ccccc2-c2nc(-c3ccccc3-n3c4ccccc4c4cc(-c5cccc6ccccc56)ccc43)nc(-c3cccc4c3sc3ccccc34)n2)cc1]

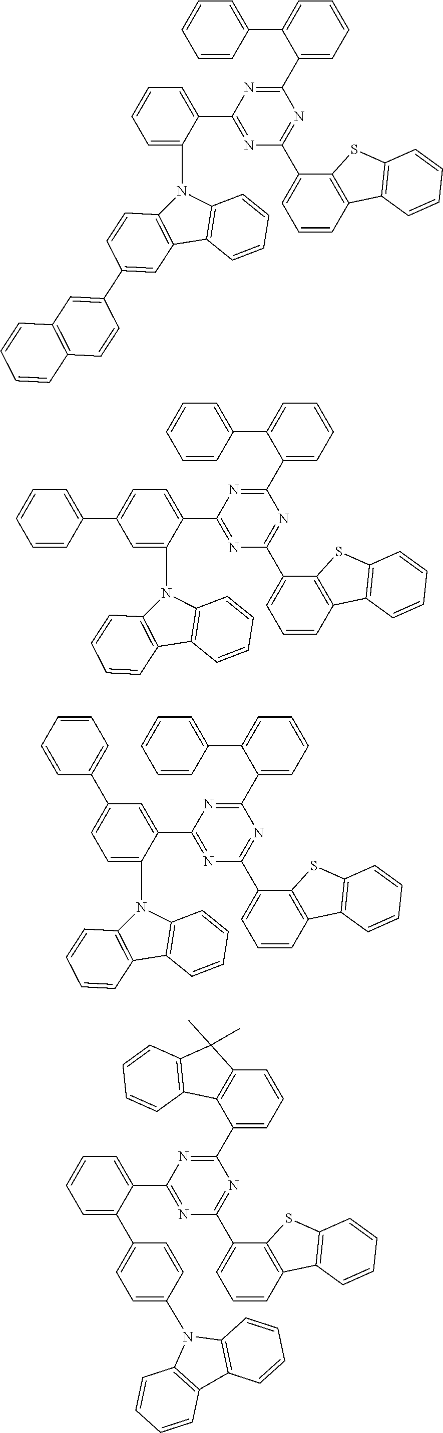

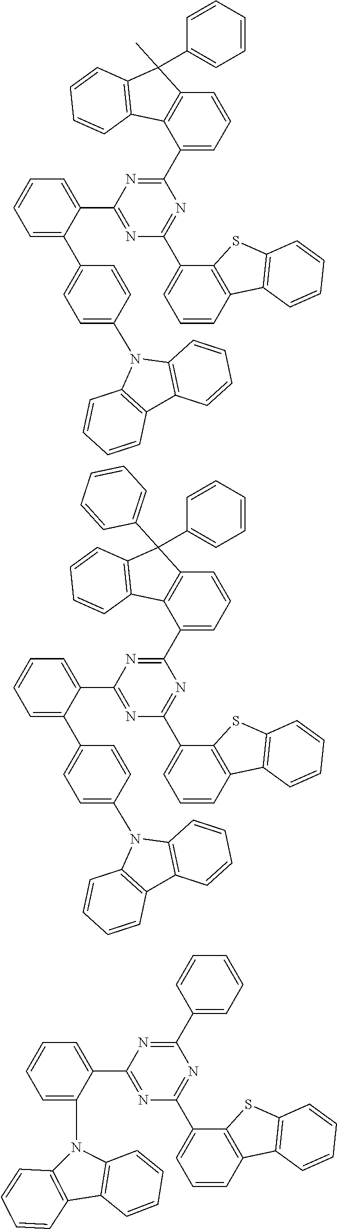

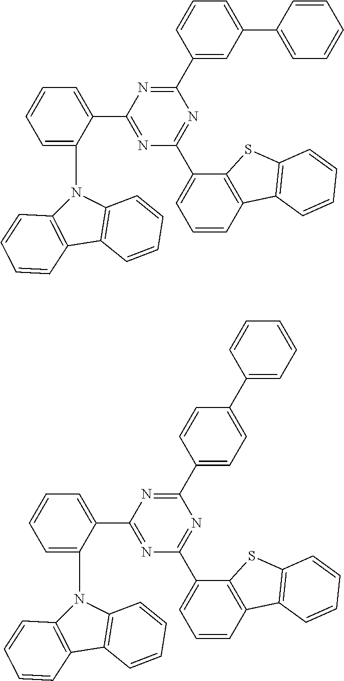 [SMILES: c1ccc(-c2cccc(-c3nc(-c4ccccc4-n4c5ccccc5c5ccccc54)nc(-c4cccc5c4sc4ccccc45)n3)c2)cc1 ; c1ccc(-c2ccc(-c3nc(-c4ccccc4-n4c5ccccc5c5ccccc54)nc(-c4cccc5c4sc4ccccc45)n3)cc2)cc1]

### <Second Compound (Second Component)>

The second component is a compound having 5 or more of the maximum number of rings, and having greater maximum number of rings than the maximum number of rings in the first compound.

Fused ring structures included in the second component are not particularly limited, and they may be a fused cyclic hydrocarbon structure only containing carbon atoms and hydrogen atoms, or may be a fused cyclic heterocycle structure containing one or two or more heteroatoms. Examples of the heteroatom in the fused cyclic heterocycle structure include O atom, S atom, N atom, B atom, and the like.

The single ring constituting the fused ring structure included in the second component is not particularly limited, and it is generally a four-membered to eight-membered single ring, and for example, a five-membered or six-membered single ring. A plurality of single rings constituting the fused ring structure may be the same as or different from each other.

Examples of the fused ring structure included in the second component include a fused ring structure constituted of one or more six-membered rings and one or more five-membered rings, a fused ring structure constituted of only two or more six-membered rings and the like.

In one embodiment, the second component includes a fused ring structure constituted of only two or more six-membered rings, and may be a compound including only the fused ring structure constituted thereof as the fused ring structure.

In one embodiment, the second component includes a fused ring structure constituted of only two or more six-membered rings, and a fused ring structure constituted of one or more six-membered rings and one or more five-membered rings, and may be a compound including only the fused ring structure constituted thereof as the fused ring structure.

In one embodiment, the second component includes a fused ring structure constituted of one or more six-membered rings and one or more five-membered rings, and may be a compound including only the fused ring structure constituted thereof as the fused ring structure.

The maximum number of rings in the second component is not particularly limited as long as it is 5 or more, and for example, it is 5 to 15.

In one embodiment, the maximum number of rings in the second component is 5 to 10.

In one embodiment, the second component has greater maximum number of rings than the maximum number of rings in the first compound by one or more, by two or more, by three or more, by four or more, or by five or more, and for example, by one or more, or by two or more.

The number of the fused ring structure included in the second component is not particularly limited, and for example, it is 1 or more and 5 or less. When a plurality of fused ring structures is included in the second component, the plurality of fused ring structures is the same as or different from each other. It may include two or more fused ring structures having the maximum number of rings in one molecule.

In one embodiment, the number of the fused ring structure included in the second component is 1, 2 or 3.

The fused ring structure included in the second component may have a substituent, and when two or more fused ring structures is included therein, a divalent or more valent group may be included between the fused ring structures. Examples of the substituents thereof include the same as the substituents described in the first component.

In one embodiment, the substituent thereof is a group having 10 or less, 9 or less, 8 or less, 7 or less, 6 or less, or 5 or less of maximum number of rings in the substituents described in the first component.

The molecular weight of the second component is not particularly limited, and for example, it is 200 to 2,000. The molecular weight of the second component may be 300 or more, 400 or more, or 500 or more, and it may also be 1,500 or less, or 1,200 or less.

In one embodiment, the second component has greater molecular weight than the molecular weight of the first component.

In one embodiment, the second component is one or more compounds selected from the group consisting of compounds represented by the following formulas (5) and (6).

In one embodiment, the second component is a compound having 5 to 10 of maximum number of rings in the compounds represented by the following formulas (5) and (6).

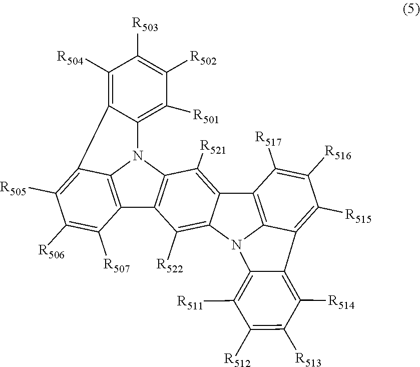

In the formula (5),
one or more sets of the adjacent two or more of R₅₀₁ to R₅₀₇ and R₅₁₁ to R₅₁₇
form a substituted or unsubstituted single ring by bonding with each other,
form a substituted or unsubstituted fused ring by bonding with each other, or
do not bond with each other;
R₅₀₁ to R₅₀₇ and R₅₁₁ to R₅₁₇ which do not form the single ring and which do not form the fused ring are independently
a hydrogen atom,
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —Si(R₉₀₁) (R₉₀₂)(R₉₀₃),
a group represented by —O—(R₉₀₄),
a group represented by —S—(R₉₀₅),
a group represented by —N(R₉₀₆)(R₉₀₇),
a halogen atom,
a cyano group,
a nitro group,
a substituted or unsubstituted aryl group having 6 to 50 ring carbon atoms, or
a substituted or unsubstituted heterocyclic group having 5 to 50 ring atoms;
R₅₂₁ and R₅₂₂ are independently
a hydrogen atom,
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —Si(R₉₀₁) (R₉₀₂)(R₉₀₃),
a group represented by —O—(R₉₀₄),
a group represented by —S—(R₉₀₅),
a group represented by —N(R₉₀₆)(R₉₀₇),
a halogen atom,
a cyano group,
a nitro group,
a substituted or unsubstituted aryl group having 6 to 50 ring carbon atoms, or
a substituted or unsubstituted heterocyclic group having 5 to 50 ring atoms; and
R₉₀₁ to R₉₀₇ are independently the same as defined for R₉₀₁ to R₉₀₇ in the formula (2).

The expression “one set of the adjacent two or more of R₅₀₁ to R₅₀₇ and R₅₁₁ to R₅₁₇” is, for example, a combination such as a set of R₅₀₁ and R₅₀₂, a set of R₅₀₂ and R₅₀₃, a set of R₅₀₃ and R₅₀₄, a set of R₅₀₅ and R₅₀₆, a set of R₅₀₆ and R₅₀₇ and a set of R₅₀₁, R₅₀₂ and R₅₀₃.

In one embodiment, at least one of, preferably two of, R₅₀₁ to R₅₀₇ and R₅₁₁ to R₅₁₇ is a group represented by —N(R₉₀₆)(R₉₀₇).

In one embodiment, R₅₀₁ to R₅₀₇ and R₅₁₁ to R₅₁₇ are independently a hydrogen atom, a substituted or unsubstituted aryl group having 6 to 50 ring carbon atoms, or a substituted or unsubstituted heterocyclic group having 5 to 50 ring atoms.

In one embodiment, the compound represented by the formula (5) is a compound represented by the following formula (52):

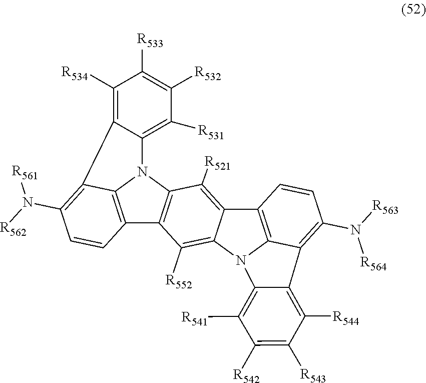

wherein in the formula (52),
one or more sets of the adjacent two or more of R₅₃₁ to R₅₃₄ and R₅₄₁ to R₅₄₄ form a substituted or unsubstituted single ring by bonding with each other, form a substituted or unsubstituted fused ring by bonding with each other, or do not bond with each other;
R₅₃₁ to R₅₃₄ and R₅₄₁ to R₅₄₄ which do not form the single ring and which do not form the fused ring, and R₅₅₁ and R₅₅₂ are independently a hydrogen atom, a substituted or unsubstituted aryl group having 6 to 50 ring carbon atoms, or a substituted or unsubstituted heterocyclic group having 5 to 50 ring atoms; and
R₅₆₁ to R₅₆₄ are independently a substituted or unsubstituted aryl group having 6 to 50 ring carbon atoms, or a substituted or unsubstituted heterocyclic group having 5 to 50 ring atoms.

In one embodiment, the compound represented by the formula (5) is a compound represented by the following formula (53):

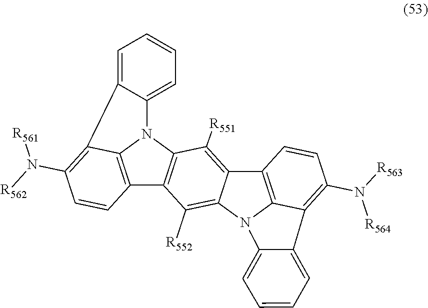

wherein in the formula (53), R₅₅₁, R₅₅₂ and R₅₆₁ to R₅₆₄ are independently the same as defined for R₅₅₁, R₅₅₂ and R₅₆₁ to R₅₆₄ in the formula (52).

In one embodiment, R₅₆₁ to R₅₆₄ in the formula (52) and the formula (53) are independently a substituted or unsubstituted aryl group having 6 to 50 ring carbon atoms (preferably a phenyl group).

In one embodiment, R₅₂₁ and R₅₂₂ in the formula (5), and R₅₅₁ and R₅₅₂ in the formula (52) and the formula (53) are hydrogen atoms.

In one embodiment, a substituent in the case of “substituted or unsubstituted” in the formula (5), the formula (52) and the formula (53) is
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted heterocyclic group having 5 to 50 ring atoms.

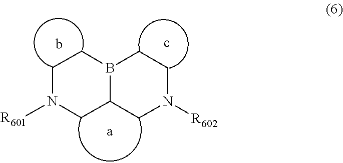

In the formula (6),
a ring a, a ring b and a ring c are independently
a substituted or unsubstituted aromatic hydrocarbon ring having 6 to 50 ring carbon atoms, or
a substituted or unsubstituted heterocycle having 5 to 50 ring atoms;
R₆₀₁ and R₆₀₂ independently form a substituted or unsubstituted heterocycle by bonding with the ring a, the ring b or the ring c, or do not form the substituted or unsubstituted heterocycle; and
R₆₀₁ and R₆₀₂ which do not form the substituted or unsubstituted heterocycle are independently
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted heterocyclic group having 5 to 50 ring atoms.

The ring a, the ring b and the ring c are a ring (a substituted or unsubstituted aromatic hydrocarbon ring having 6 to 50 ring carbon atoms, or a substituted or unsubstituted heterocycle having 5 to 50 ring atoms) fused with the fused two-ring structure constituted of a boron atom and two nitrogen atoms in the middle of the formula (6).

The “aromatic hydrocarbon ring” of the ring a, the ring b and the ring c is the same structure as a compound in which hydrogen atoms are introduced in the above-mentioned “aryl group”.

The “aromatic hydrocarbon ring” of the ring a includes three carbon atoms in the fused two-ring structure in the middle of the formula (6) as ring atoms.

The “aromatic hydrocarbon ring” of the ring b and the ring c includes two carbon atoms in the fused two-ring structure in the middle of the formula (6) as ring atoms.

Specific examples of the “substituted or unsubstituted aromatic hydrocarbon ring having 6 to 50 ring carbon atoms” include a compound in which hydrogen atoms are introduced in the “aryl group” described in the specific example group G1, and the like.

The “heterocycle” of the ring a, the ring b and the ring c is the same structure as a compound in which hydrogen atoms are introduced in the above-mentioned “heterocyclic group”.

The “heterocycle” of the ring a includes three carbon atoms in the fused two-ring structure in the middle of the formula (6) as ring atoms. The “heterocycle” of the ring b and the ring c includes two carbon atoms in the fused two-ring structure in the middle of the formula (6) as ring atoms. Specific examples of the “substituted or unsubstituted heterocycle having 5 to 50 ring atoms” include a compound in which hydrogen atoms are introduced in the “heterocyclic group” described in the specific example group G2, and the like.

R₆₀₁ and R₆₀₂ independently may form a substituted or unsubstituted heterocycle by bonding with the ring a, the ring b or the ring c. The heterocycle in this case includes a nitrogen atom in the fused two-ring structure in the middle of the formula (6). The heterocycle in this case may include a heteroatom other than the nitrogen atom. The expression “R₆₀₁ and R₆₀₂ bond with the ring a, the ring b or the ring c” means that specifically, an atom constituting the ring a, the ring b or the ring c and an atom constituting R₆₀₁ and R₆₀₂ are bonded. For example, R₆₀₁ may be bonded with the ring a to form a two-ring-fused (or three or more-ring-fused) nitrogen-containing heterocycle in which a ring containing R₆₀₁ and the ring a are fused. Specific examples of the nitrogen-containing heterocycle include a compound corresponding to a heterocyclic group containing a nitrogen atom and fusing two or more rings, and the like in the specific example group G2.

The case where R₆₀₁ is bonded with the ring b, the case where R₆₀₂ is bonded with the ring a, and the case where R₆₀₂ is bonded with the ring c are also the same as the case described above.

In one embodiment, the ring a, the ring b and the ring c in the formula (6) are independently a substituted or unsubstituted aromatic hydrocarbon ring having 6 to 50 ring carbon atoms.

In one embodiment, the ring a, the ring b and the ring c in the formula (6) are independently a substituted or unsubstituted benzene ring or naphthalene ring.

In one embodiment, R₆₀₁ and R₆₀₂ in the formula (6) are independently a substituted or unsubstituted aryl group having 6 to 50 ring carbon atoms, or a substituted or unsubstituted heterocyclic group having 5 to 50 ring atoms, and preferably a substituted or unsubstituted aryl group having 6 to 50 ring carbon atoms.

In one embodiment, the compound represented by the formula (6) is a compound represented by the following formula (62).

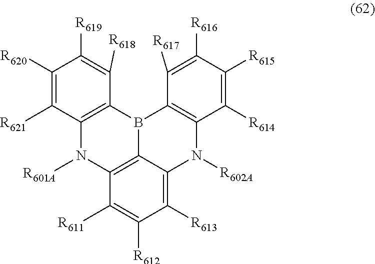

In the formula (62),
R_{601A} forms a substituted or unsubstituted heterocycle by bonding with one or more groups selected from the group consisting of R₆₁₁ and R₆₂₁, or does not form the substituted or unsubstituted heterocycle;
R_{602A} forms a substituted or unsubstituted heterocycle by bonding with one or more groups selected from the group consisting of R₆₁₃ and R₆₁₄, or does not form the substituted or unsubstituted heterocycle;
R_{601A} and R_{602A} which do not form the substituted or unsubstituted heterocycle are independently
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted heterocyclic group having 5 to 50 ring atoms;
one or more sets of the adjacent two or more of R₆₁₁ to R₆₂₁
form a substituted or unsubstituted single ring by bonding with each other,
form a substituted or unsubstituted fused ring by bonding with each other, or
do not bond with each other; and
R₆₁₁ to R₆₂₁ which do not form the substituted or unsubstituted heterocycle, which do not form the single ring and which do not form the fused ring are independently
a hydrogen atom,
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —Si(R₉₀₁) (R₉₀₂)(R₉₀₃),
a group represented by —O—(R₉₀₄),
a group represented by —S—(R₉₀₅),
a group represented by —N(R₉₀₆)(R₉₀₇),
a halogen atom,
a cyano group,
a nitro group,
a substituted or unsubstituted aryl group having 6 to 50 ring carbon atoms, or
a substituted or unsubstituted heterocyclic group having 5 to 50 ring atoms.

R_{601A} and R_{602A} in the formula (62) are independently a group corresponding to R₆₀₁ and R₆₀₂ in the formula (6).

For example, R_{601A} and R₆₁₁ may be bonded to form a two-ring-fused (or three or more-ring-fused) nitrogen-containing heterocycle in which a ring containing them and a benzene ring corresponding to the ring a are fused. Specific examples of the nitrogen-containing heterocycle include a compound corresponding to a heterocyclic group containing a nitrogen atom and fusing two or more rings, and the like in the specific example group G2. The case where R_{601A} and R₆₂₁ are bonded, the case where R_{602A} and R₆₁₃ are bonded, and the case where R_{602A} and R₆₁₄ are bonded are also the same as the case described above.

One or more sets of the adjacent two or more of R₆₁₁ to R₆₂₁ may form a substituted or unsubstituted single ring by bonding with each other, or form a substituted or unsubstituted fused ring by bonding with each other.

For example, R₆₁₁ and R₆₁₂ may be bonded in terms of a six-membered ring in which they are bonded to form a structure in which a benzene ring, an indole ring, a pyrrole ring, a benzofuran ring, a benzothiophene ring, or the like are fused, and the formed fused ring is a naphthalene ring, a carbazole ring, an indole ring, a dibenzofuran ring or a dibenzothiophene ring.

In one embodiment, R₆₁₁ to R₆₂₁ which do not form the ring are independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

In one embodiment, R₆₁₁ to R₆₂₁ which do not form the ring are independently a hydrogen atom, a substituted or unsubstituted aryl group having 6 to 50 ring carbon atoms, or a substituted or unsubstituted heterocyclic group having 5 to 50 ring atoms.

In one embodiment, R₆₁₁ to R₆₂₁ which do not form the ring are independently a hydrogen atom, or a substituted or unsubstituted alkyl group having 1 to 50 carbon atoms.

In one embodiment, R₆₁₁ to R₆₂₁ which do not form the ring are independently a hydrogen atom, or a substituted or unsubstituted alkyl group having 1 to 50 carbon atoms, and at least one of R₆₁₁ to R₆₂₁ is a substituted or unsubstituted alkyl group having 1 to 50 carbon atoms.

In one embodiment, the compound represented by the formula (62) is a compound represented by the following formula (63):

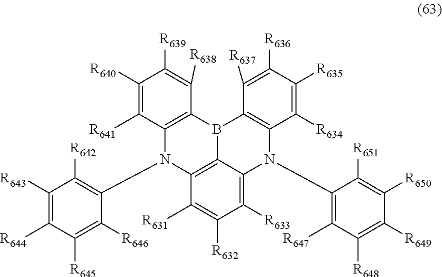

wherein in the formula (63),
R₆₃₁ forms a substituted or unsubstituted heterocycle by bonding with R₆₄₆, or does not form the substituted or unsubstituted heterocycle;
R₆₃₃ forms a substituted or unsubstituted heterocycle by bonding with R₆₄₇, or does not form the substituted or unsubstituted heterocycle;
R₆₃₄ forms a substituted or unsubstituted heterocycle by bonding with R₆₅₁, or does not form the substituted or unsubstituted heterocycle;
R₆₄₁ forms a substituted or unsubstituted heterocycle by bonding with R₆₄₂, or does not form the substituted or unsubstituted heterocycle;
one or more sets of the adjacent two or more of R₆₃₁ to R₆₅₁ form a substituted or unsubstituted single ring by bonding with each other, form a substituted or unsubstituted fused ring by bonding with each other, or do not bond with each other; and
R₆₃₁ to R₆₅₁ which do not form the substituted or unsubstituted heterocycle, which do not form the single ring and which do not form the fused ring are independently
a hydrogen atom,
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —Si(R₉₀₁) (R₉₀₂)(R₉₀₃),
a group represented by —O—(R₉₀₄),
a group represented by —S—(R₉₀₅),
a group represented by —N(R₉₀₆)(R₉₀₇),
a halogen atom,
a cyano group,
a nitro group,
a substituted or unsubstituted aryl group having 6 to 50 ring carbon atoms, or
a substituted or unsubstituted heterocyclic group having 5 to 50 ring atoms.

R₆₃₁ may form a substituted or unsubstituted heterocycle by bonding with R₆₄₆. For example, R₆₃₁ and R₆₄₆ may be bonded to form a three or more-ring-fused nitrogen-containing heterocycle in which a benzene ring which R₆₄₆ is bonded with, a ring containing N and a benzene ring corresponding to the ring a are fused. Specific examples of the nitrogen-containing heterocycle include a compound corresponding to a heterocyclic group containing a nitrogen atom and fusing three or more rings, and the like in the specific example group G2. The case where R₆₃₃ and R₆₄₇ are bonded, the case where R₆₃₄ and R₆₅₁ are bonded, and the case where R₆₄₁ and R₆₄₂ are bonded are also the same as the case described above.

In one embodiment, R₆₃₁ to R₆₅₁ which do not form the ring are independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

In one embodiment, R₆₃₁ to R₆₅₁ which do not form the ring are independently a hydrogen atom, a substituted or unsubstituted aryl group having 6 to 50 ring carbon atoms, or a substituted or unsubstituted heterocyclic group having 5 to 50 ring atoms.

In one embodiment, R₆₃₁ to R₆₅₁ which do not form the ring are independently a hydrogen atom, or a substituted or unsubstituted alkyl group having 1 to 50 carbon atoms.

In one embodiment, R₆₃₁ to R₆₅₁ which do not form the ring are independently a hydrogen atom, or a substituted or unsubstituted alkyl group having 1 to 50 carbon atoms, and at least one of R₆₃₁ to R₆₅₁ is a substituted or unsubstituted alkyl group having 1 to 50 carbon atoms.

In one embodiment, the second component is a compound in which the orientation order parameter S′ obtained by the following formula is 0.8 or more in a thin film formed using a vacuum deposition process:

$${S}^{′} = 1 < cos 2 θ >= 2 ko / ( ke + 2 k o ) = 2 / 3 ( 1 - S )$$

$$S = ( 1 / 2 ) < 3 cos 2 θ - 1 > = ( k e - ko ) / ( ke + 2 k o )$$

wherein θ is the angle between the molecular axis in the thin film and the normal direction to a substrate; ko is the extinction coefficient in the direction parallel to the substrate obtained by multi-incident angle spectroscopic ellipsometry measurement of the thin film; and ke is the extinction coefficient in the vertical direction thereto obtained by multi-incident angle spectroscopic ellipsometry measurement of the thin film.

The film of low molecular material deposited in vacuum on a substrate has generally uniaxial rotational symmetry with the normal direction to the substrate as the axis of rotation. The orientation order parameter S′ obtained by multi-incident angle spectroscopic ellipsometry measurement is 1.0, when all molecules are parallelly oriented parallel to the substrate. In addition, it is 0.66, when all molecules are in random without orientation. The method for evaluation thereof is a known method, and is described in detail in Organic Electronics, vol. 10, pp. 127 (2009). Further, the method for forming a thin film formed is a vacuum deposition process.

The method for measuring the orientation order parameter S′ is specifically shown as follows.

A measurement target material is deposited in vacuum on a glass substrate to form a film having the thickness of about 50 nm, and a measurement is conducted every 5° within the measurement angle range of 45° to 75° using a spectroscopic ellipsometry apparatus (manufactured by J. A. Woollam Co. (US), M-2000UI). In order to improve the measurement accuracy of the extinction coefficient, the transmission spectrum in the direction normal to the substrate (perpendicular to the surface of the substrate) is measured using the apparatus in addition thereto. Only the glass substrate is subjected to the measurement in the same manner. Fitting is conducted using the analysis software (CompleteEASE) manufactured by the company on the obtained measurement information. As the fitting conditions, an anisotropic model is used with uniaxial rotational symmetry, and the parameter MSE indicating the root mean square error in the software is set to 3.0 or less, and then an order parameter of the organic film formed on the substrate is calculated. The order parameter is calculated based on the peak wavelength of the singlet energy S1 on the longer wavelength side of the extinction coefficient (in-plane direction). An isotropic model is used for the glass substrate.

In one embodiment, the orientation order parameter S′ of the second component is 0.85 or more, or 0.90 or more.

Specific examples of the second component include the following compounds. Here, the present invention is not limited to these specific examples.

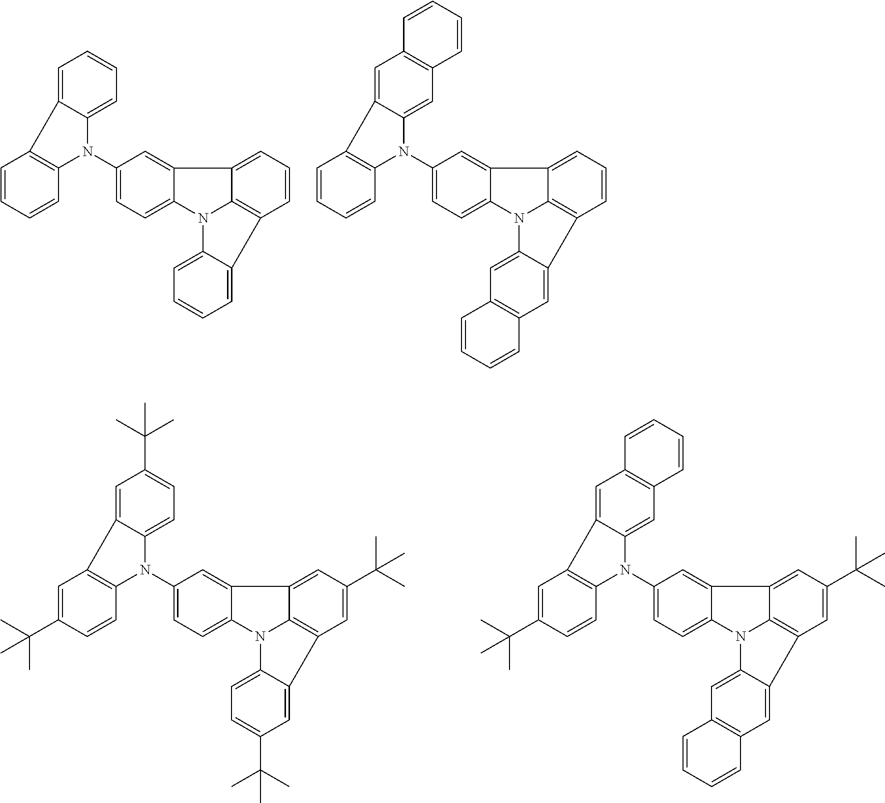

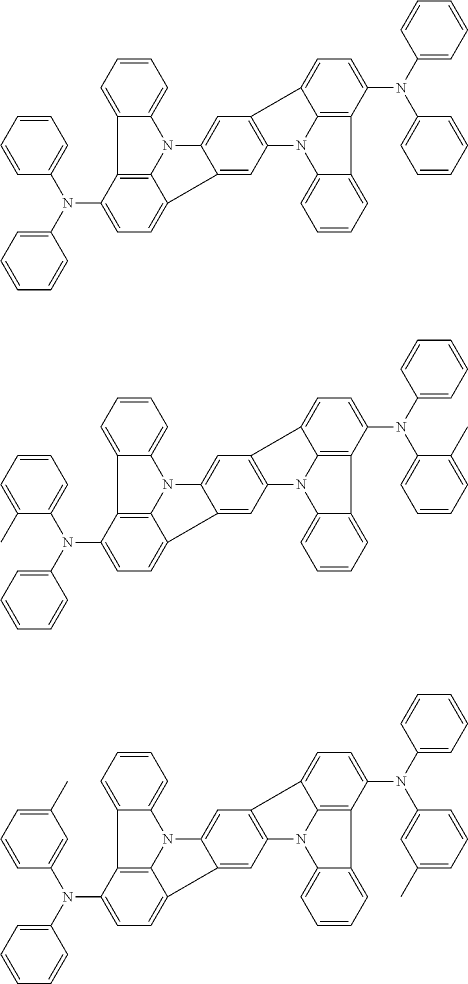 [SMILES: c1ccc(N(c2ccccc2)c2ccc3c4cc5c(cc4n4c6ccccc6c2c34)c2ccc(N(c3ccccc3)c3ccccc3)c3c4ccccc4n5c23)cc1 ; Cc1ccccc1N(c1ccccc1)c1ccc2c3cc4c(cc3n3c5ccccc5c1c23)c1ccc(N(c2ccccc2)c2ccccc2C)c2c3ccccc3n4c12 ; Cc1cccc(N(c2ccccc2)c2ccc3c4cc5c(cc4n4c6ccccc6c2c34)c2ccc(N(c3ccccc3)c3cccc(C)c3)c3c4ccccc4n5c23)c1]

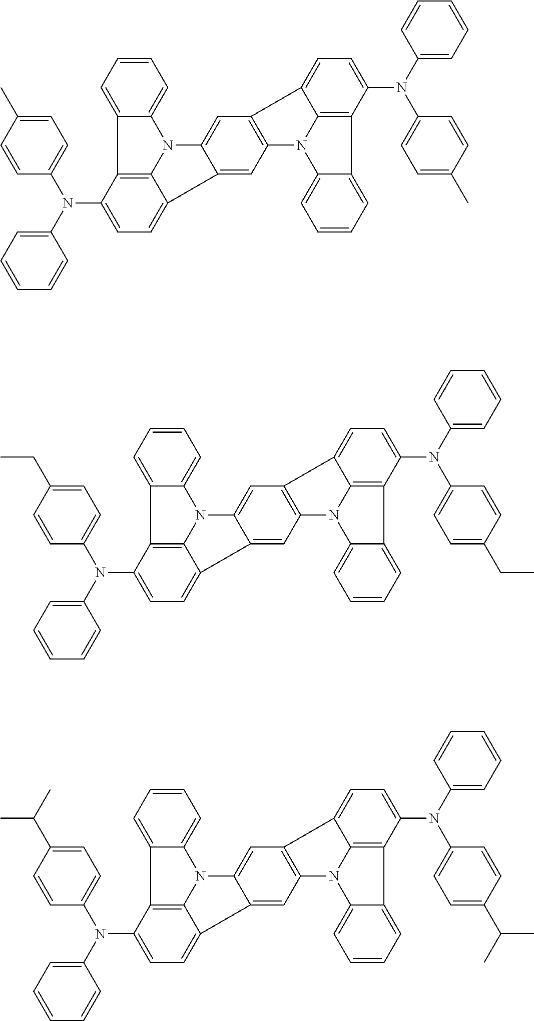

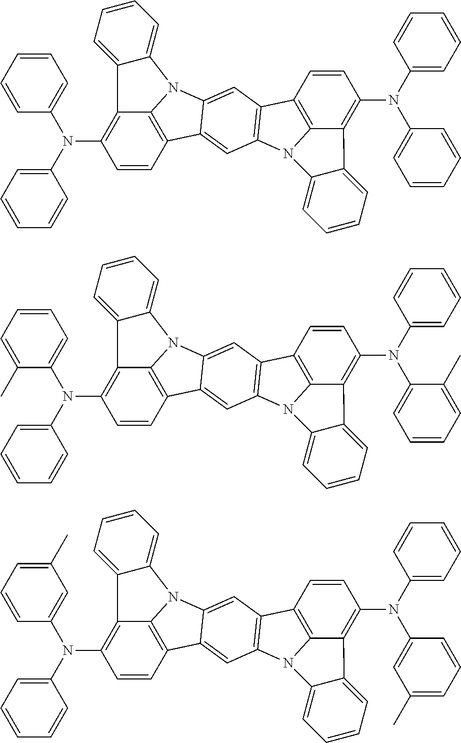 [SMILES: c1ccc(N(c2ccccc2)c2ccc3c4cc5c(cc4n4c6ccccc6c2c34)c2ccc(N(c3ccccc3)c3ccccc3)c3c4ccccc4n5c23)cc1 ; Cc1ccccc1N(c1ccccc1)c1ccc2c3cc4c(cc3n3c5ccccc5c1c23)c1ccc(N(c2ccccc2)c2ccccc2C)c2c3ccccc3n4c12 ; Cc1cccc(N(c2ccccc2)c2ccc3c4cc5c(cc4n4c6ccccc6c2c34)c2ccc(N(c3ccccc3)c3cccc(C)c3)c3c4ccccc4n5c23)c1]

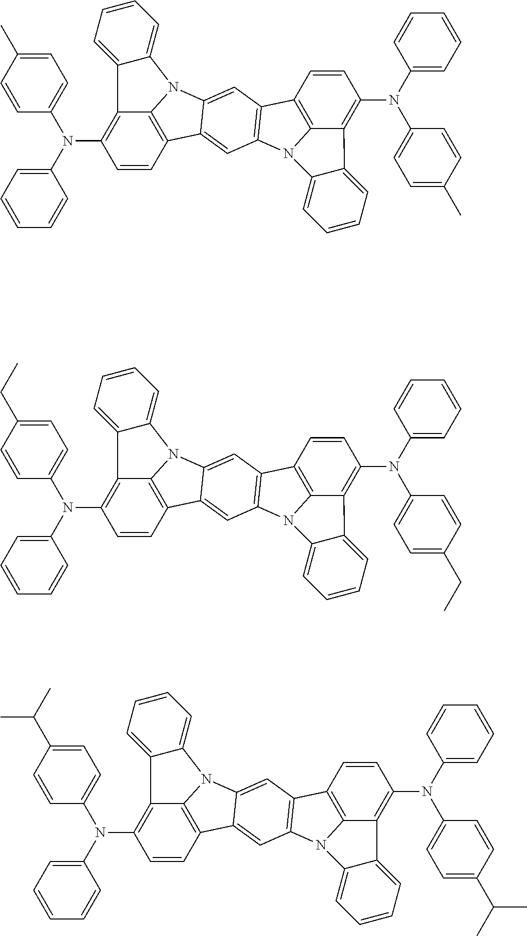

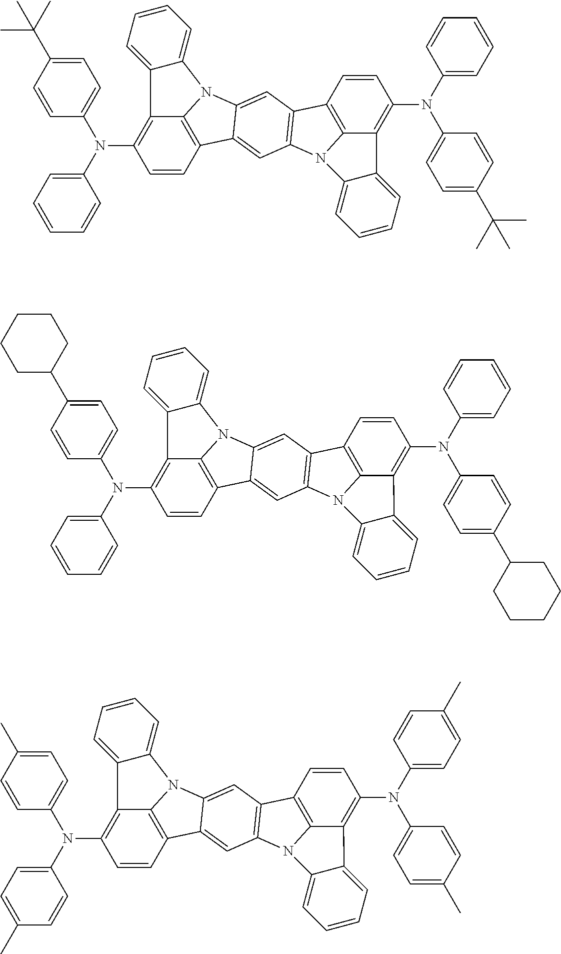 [SMILES: CC(C)(C)c1ccc(N(c2ccccc2)c2ccc3c4cc5c(cc4n4c6ccccc6c2c34)c2ccc(N(c3ccccc3)c3ccc(C(C)(C)C)cc3)c3c4ccccc4n5c23)cc1 ; c1ccc(N(c2ccc(C3CCCCC3)cc2)c2ccc3c4cc5c(cc4n4c6ccccc6c2c34)c2ccc(N(c3ccccc3)c3ccc(C4CCCCC4)cc3)c3c4ccccc4n5c23)cc1 ; Cc1ccc(N(c2ccc(C)cc2)c2ccc3c4cc5c(cc4n4c6ccccc6c2c34)c2ccc(N(c3ccc(C)cc3)c3ccc(C)cc3)c3c4ccccc4n5c23)cc1]

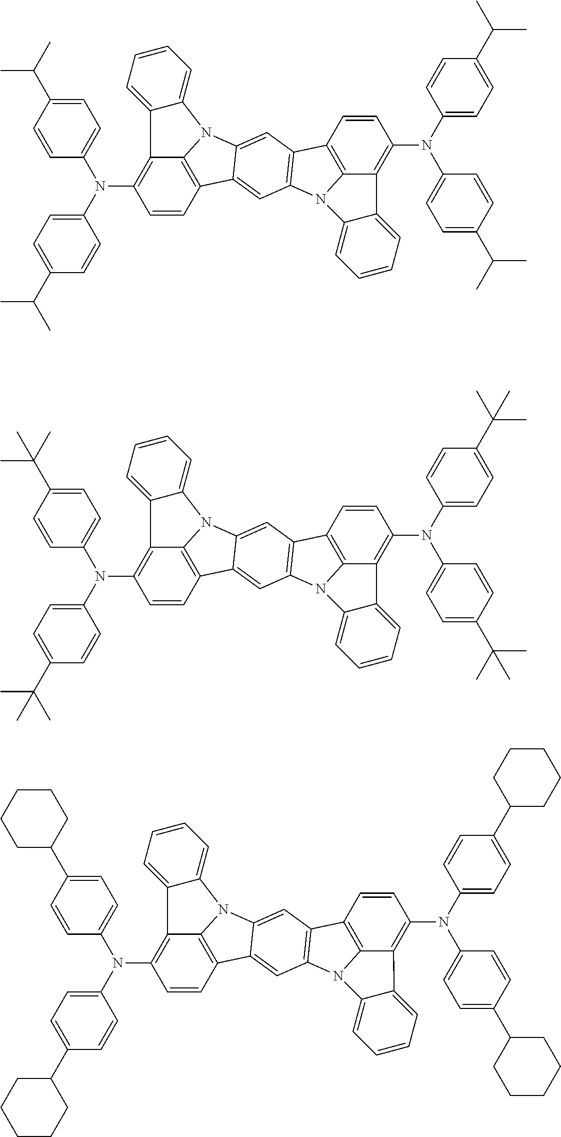

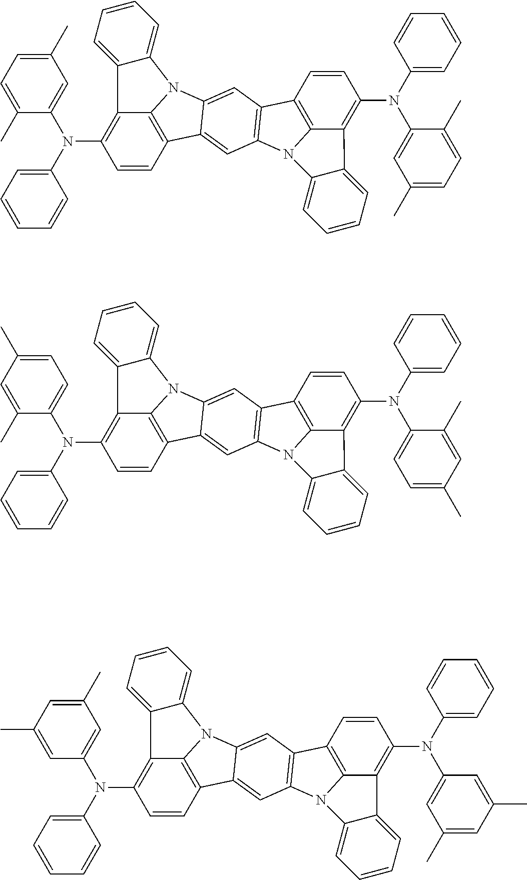

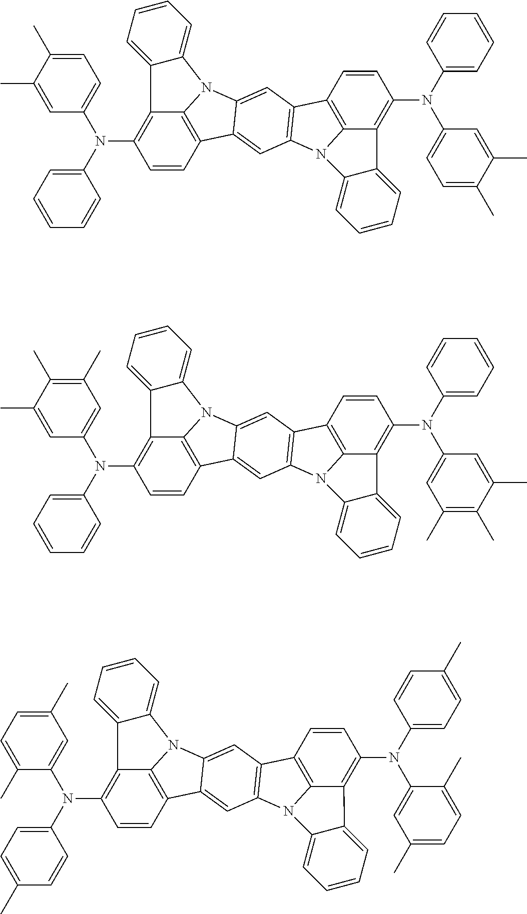

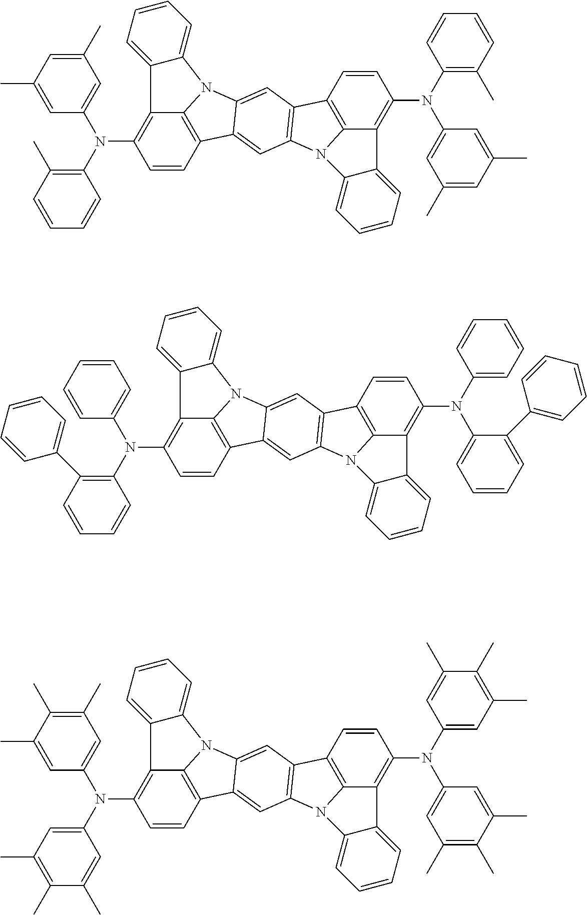

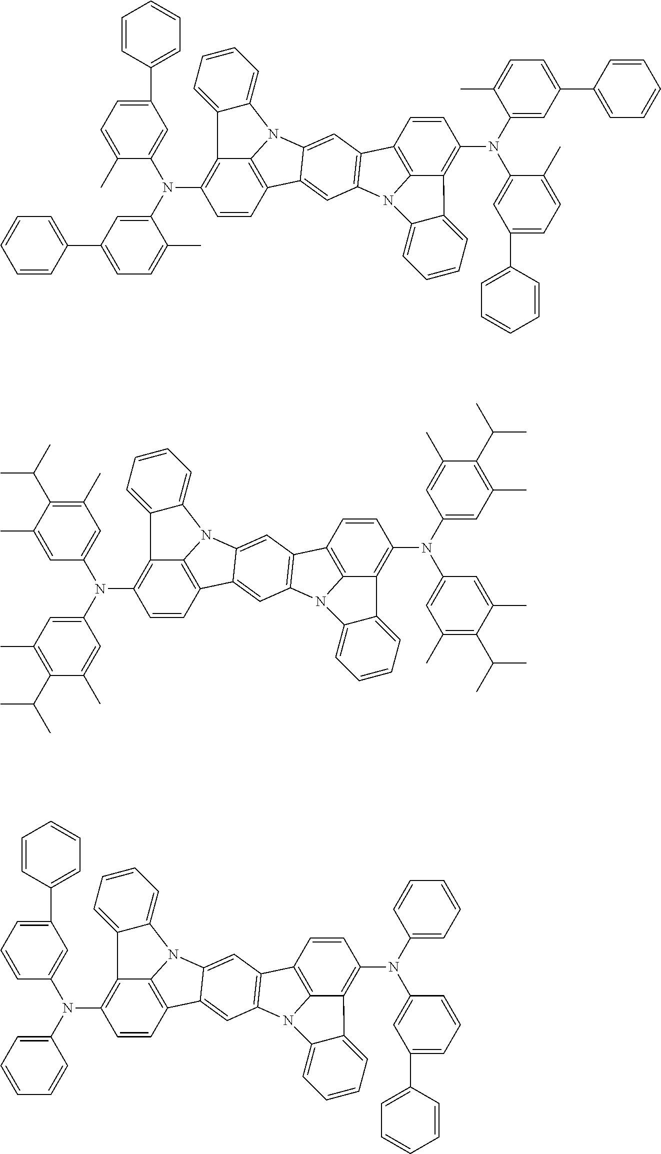 [SMILES: Cc1ccc(-c2ccccc2)cc1N(c1cc(-c2ccccc2)ccc1C)c1ccc2c3cc4c(cc3n3c5ccccc5c1c23)c1ccc(N(c2cc(-c3ccccc3)ccc2C)c2cc(-c3ccccc3)ccc2C)c2c3ccccc3n4c12 ; Cc1cc(N(c2cc(C)c(C(C)C)c(C)c2)c2ccc3c4cc5c(cc4n4c6ccccc6c2c34)c2ccc(N(c3cc(C)c(C(C)C)c(C)c3)c3cc(C)c(C(C)C)c(C)c3)c3c4ccccc4n5c23)cc(C)c1C(C)C ; c1ccc(-c2cccc(N(c3ccccc3)c3ccc4c5cc6c(cc5n5c7ccccc7c3c45)c3ccc(N(c4ccccc4)c4cccc(-c5ccccc5)c4)c4c5ccccc5n6c34)c2)cc1]

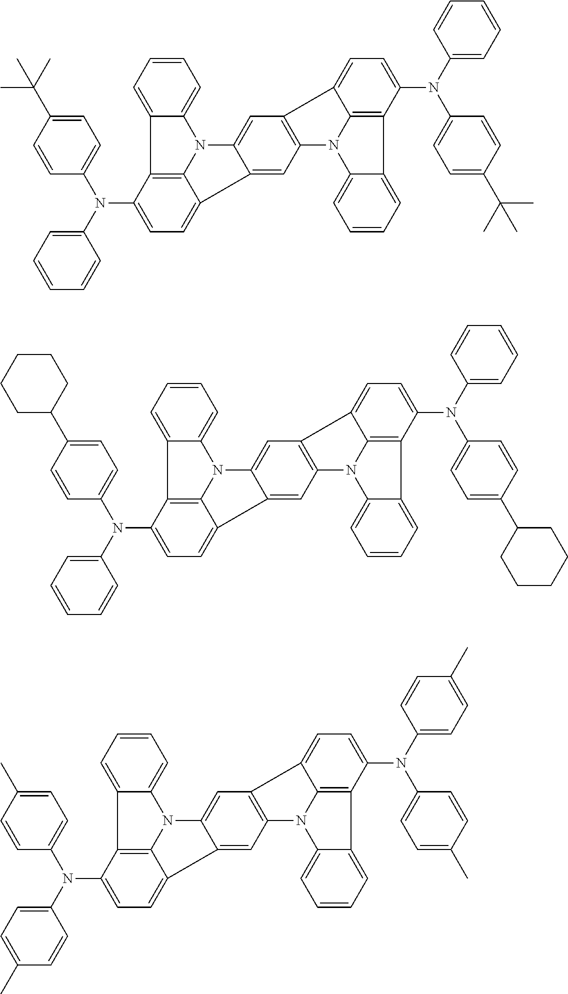 [SMILES: CC(C)(C)c1ccc(N(c2ccccc2)c2ccc3c4cc5c(cc4n4c6ccccc6c2c34)c2ccc(N(c3ccccc3)c3ccc(C(C)(C)C)cc3)c3c4ccccc4n5c23)cc1 ; c1ccc(N(c2ccc(C3CCCCC3)cc2)c2ccc3c4cc5c(cc4n4c6ccccc6c2c34)c2ccc(N(c3ccccc3)c3ccc(C4CCCCC4)cc3)c3c4ccccc4n5c23)cc1 ; Cc1ccc(N(c2ccc(C)cc2)c2ccc3c4cc5c(cc4n4c6ccccc6c2c34)c2ccc(N(c3ccc(C)cc3)c3ccc(C)cc3)c3c4ccccc4n5c23)cc1]

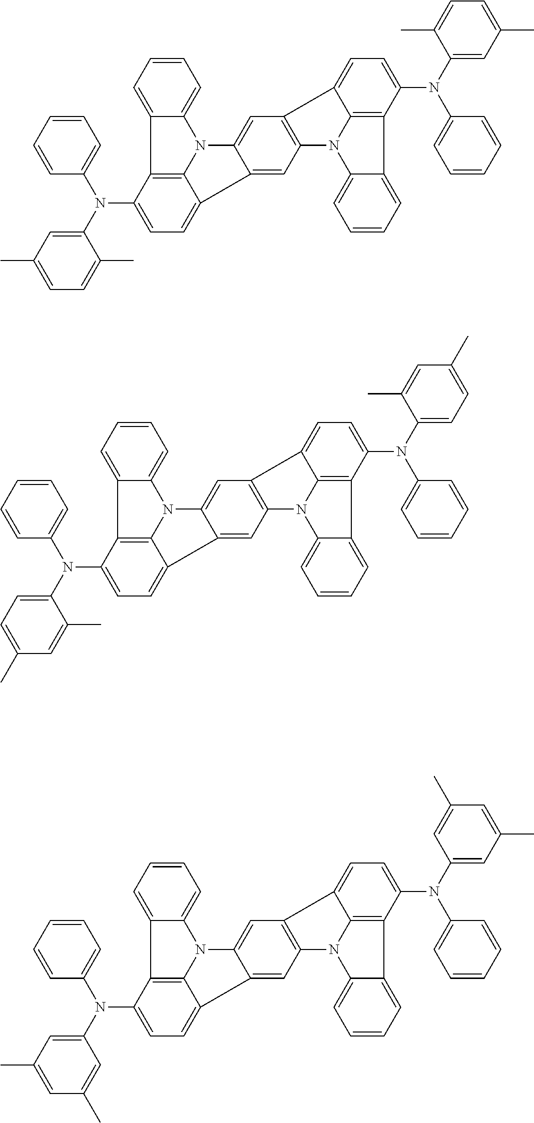

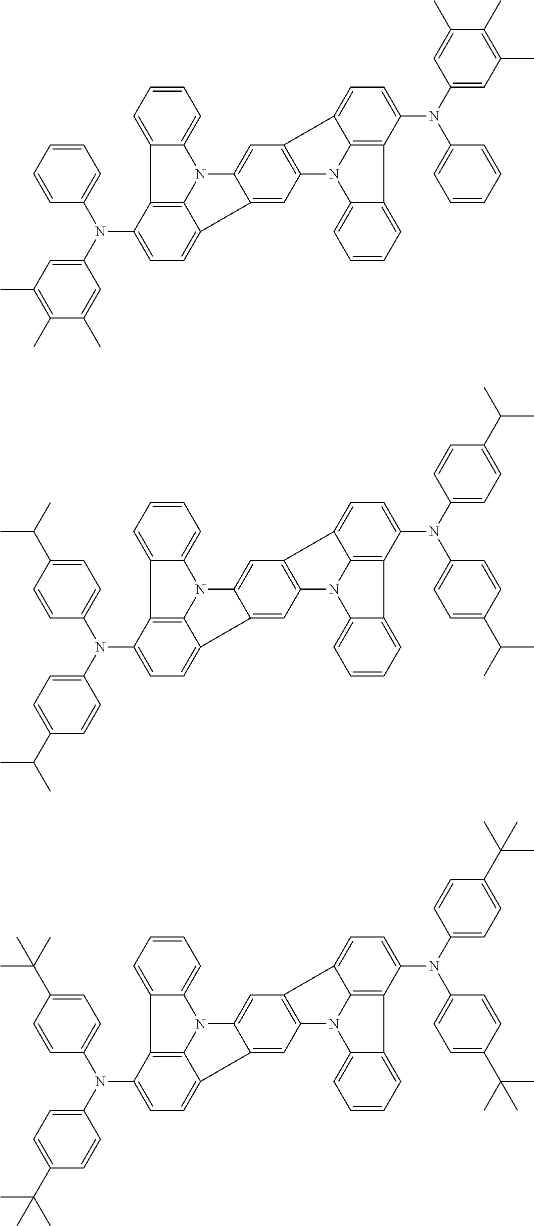

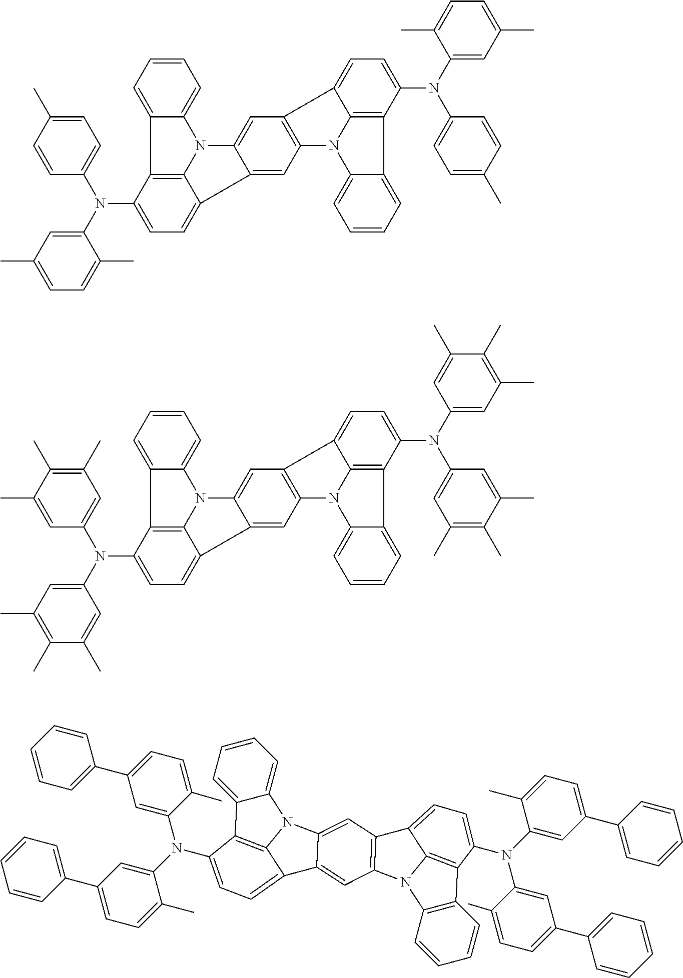

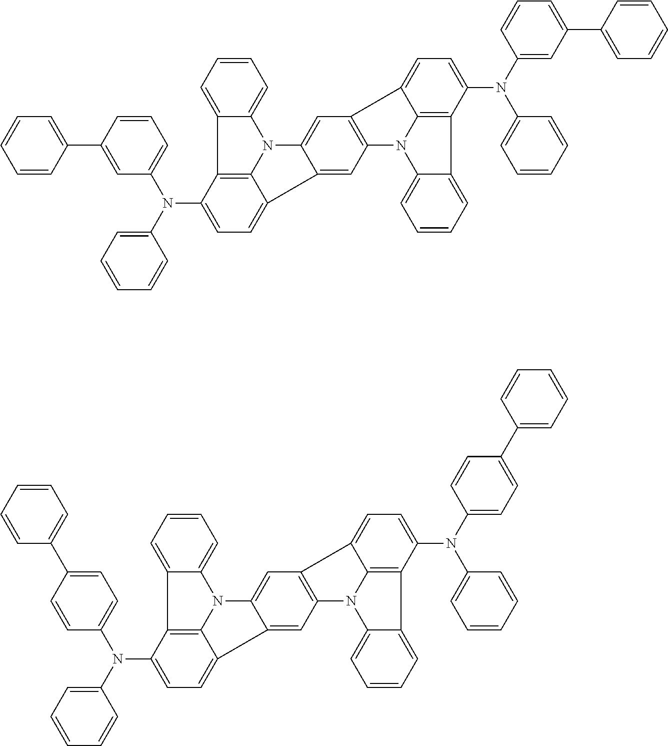 [SMILES: c1ccc(-c2cccc(N(c3ccccc3)c3ccc4c5cc6c(cc5n5c7ccccc7c3c45)c3ccc(N(c4ccccc4)c4cccc(-c5ccccc5)c4)c4c5ccccc5n6c34)c2)cc1 ; c1ccc(-c2ccc(N(c3ccccc3)c3ccc4c5cc6c(cc5n5c7ccccc7c3c45)c3ccc(N(c4ccccc4)c4ccc(-c5ccccc5)cc4)c4c5ccccc5n6c34)cc2)cc1]

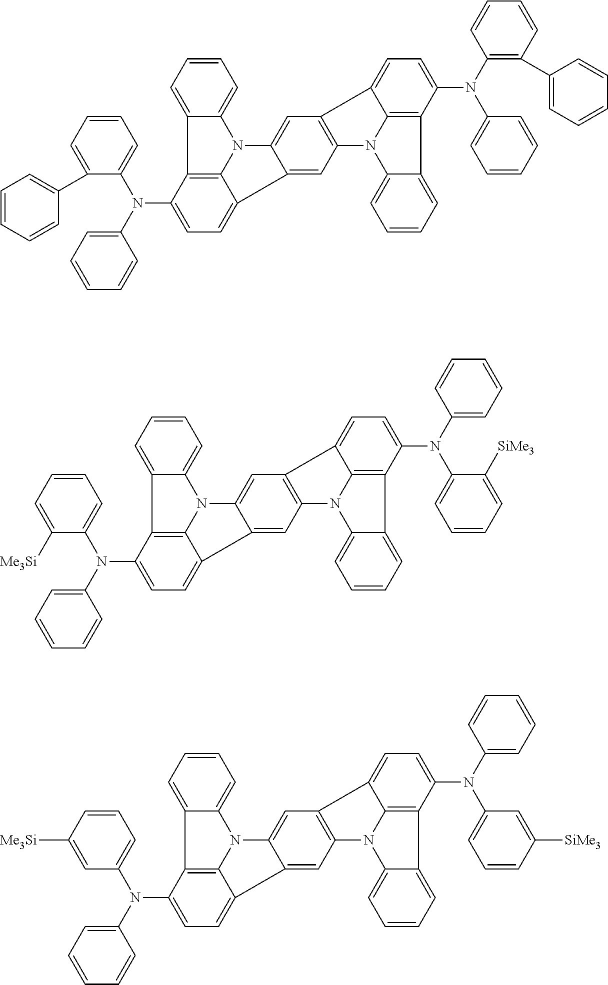 [SMILES: c1ccc(-c2ccccc2N(c2ccccc2)c2ccc3c4cc5c(cc4n4c6ccccc6c2c34)c2ccc(N(c3ccccc3)c3ccccc3-c3ccccc3)c3c4ccccc4n5c23)cc1 ; C[Si](C)(C)c1ccccc1N(c1ccccc1)c1ccc2c3cc4c(cc3n3c5ccccc5c1c23)c1ccc(N(c2ccccc2)c2ccccc2[Si](C)(C)C)c2c3ccccc3n4c12 ; C[Si](C)(C)c1cccc(N(c2ccccc2)c2ccc3c4cc5c(cc4n4c6ccccc6c2c34)c2ccc(N(c3ccccc3)c3cccc([Si](C)(C)C)c3)c3c4ccccc4n5c23)c1]

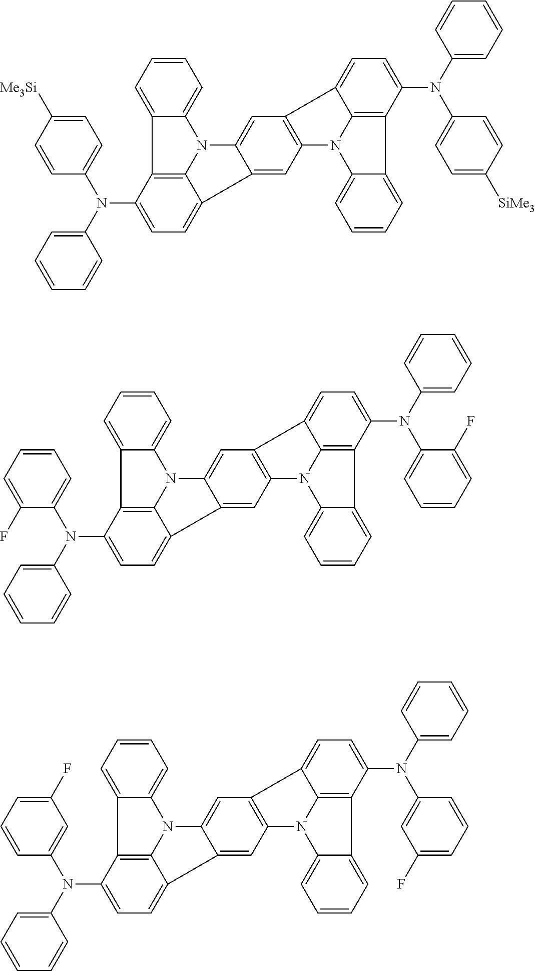 [SMILES: C[Si](C)(C)c1ccc(N(c2ccccc2)c2ccc3c4cc5c(cc4n4c6ccccc6c2c34)c2ccc(N(c3ccccc3)c3ccc([Si](C)(C)C)cc3)c3c4ccccc4n5c23)cc1 ; Fc1ccccc1N(c1ccccc1)c1ccc2c3cc4c(cc3n3c5ccccc5c1c23)c1ccc(N(c2ccccc2)c2ccccc2F)c2c3ccccc3n4c12 ; Fc1cccc(N(c2ccccc2)c2ccc3c4cc5c(cc4n4c6ccccc6c2c34)c2ccc(N(c3ccccc3)c3cccc(F)c3)c3c4ccccc4n5c23)c1]

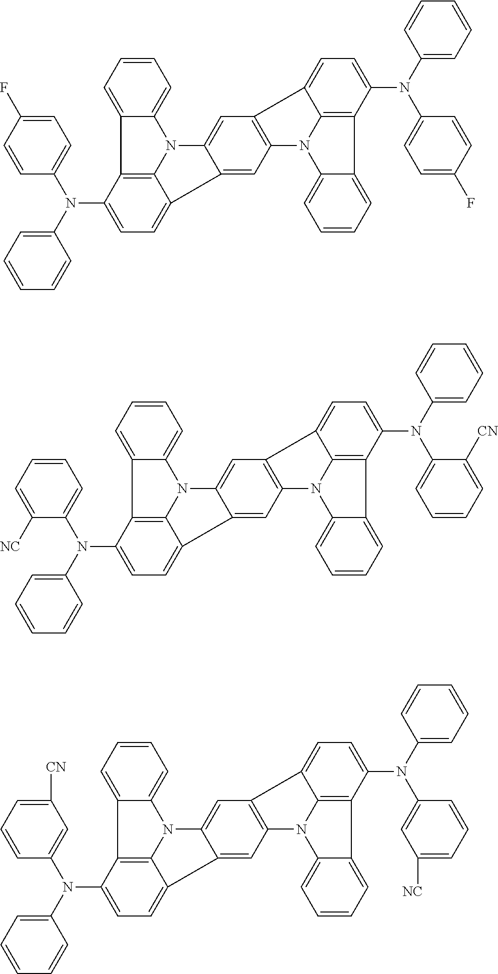 [SMILES: Fc1ccc(N(c2ccccc2)c2ccc3c4cc5c(cc4n4c6ccccc6c2c34)c2ccc(N(c3ccccc3)c3ccc(F)cc3)c3c4ccccc4n5c23)cc1 ; N#Cc1ccccc1N(c1ccccc1)c1ccc2c3cc4c(cc3n3c5ccccc5c1c23)c1ccc(N(c2ccccc2)c2ccccc2C#N)c2c3ccccc3n4c12 ; N#Cc1cccc(N(c2ccccc2)c2ccc3c4cc5c(cc4n4c6ccccc6c2c34)c2ccc(N(c3ccccc3)c3cccc(C#N)c3)c3c4ccccc4n5c23)c1]

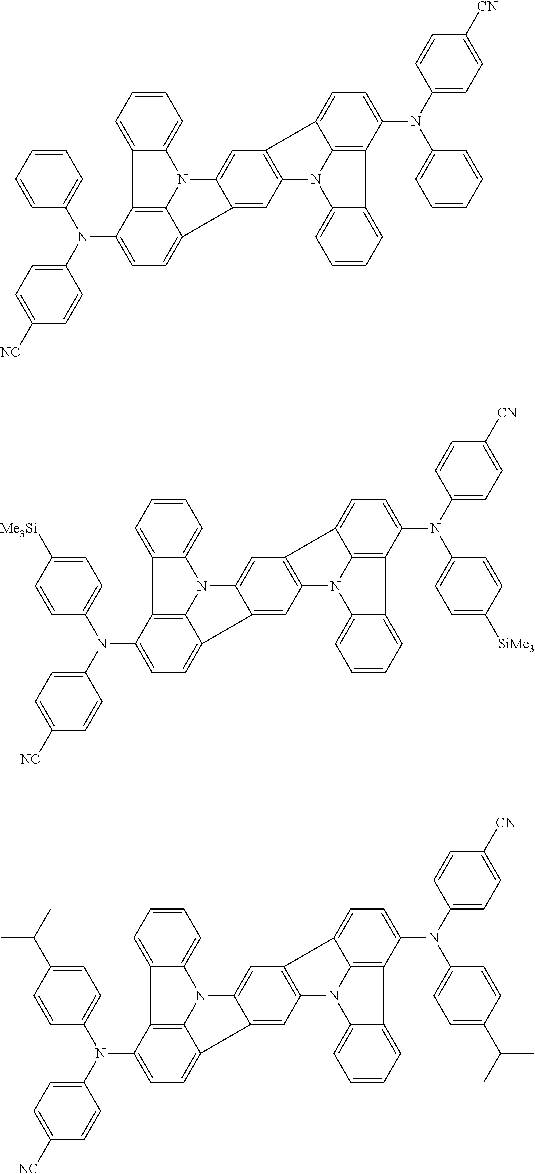 [SMILES: N#Cc1ccc(N(c2ccccc2)c2ccc3c4cc5c(cc4n4c6ccccc6c2c34)c2ccc(N(c3ccccc3)c3ccc(C#N)cc3)c3c4ccccc4n5c23)cc1 ; C[Si](C)(C)c1ccc(N(c2ccc(C#N)cc2)c2ccc3c4cc5c(cc4n4c6ccccc6c2c34)c2ccc(N(c3ccc(C#N)cc3)c3ccc([Si](C)(C)C)cc3)c3c4ccccc4n5c23)cc1 ; CC(C)c1ccc(N(c2ccc(C#N)cc2)c2ccc3c4cc5c(cc4n4c6ccccc6c2c34)c2ccc(N(c3ccc(C#N)cc3)c3ccc(C(C)C)cc3)c3c4ccccc4n5c23)cc1]

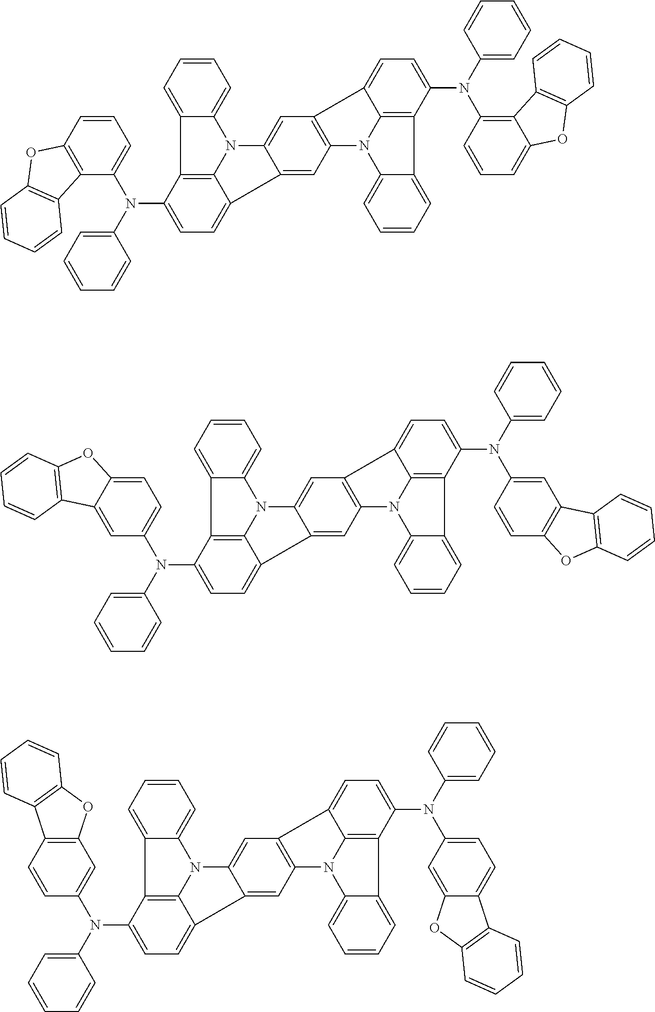 [SMILES: c1ccc(N(c2cccc3oc4ccccc4c23)c2ccc3c4cc5c(cc4n4c6ccccc6c2c34)c2ccc(N(c3ccccc3)c3cccc4oc6ccccc6c34)c3c4ccccc4n5c23)cc1 ; c1ccc(N(c2ccc3oc4ccccc4c3c2)c2ccc3c4cc5c(cc4n4c6ccccc6c2c34)c2ccc(N(c3ccccc3)c3ccc4oc6ccccc6c4c3)c3c4ccccc4n5c23)cc1 ; c1ccc(N(c2ccc3c(c2)oc2ccccc23)c2ccc3c4cc5c(cc4n4c6ccccc6c2c34)c2ccc(N(c3ccccc3)c3ccc4c(c3)oc3ccccc34)c3c4ccccc4n5c23)cc1]

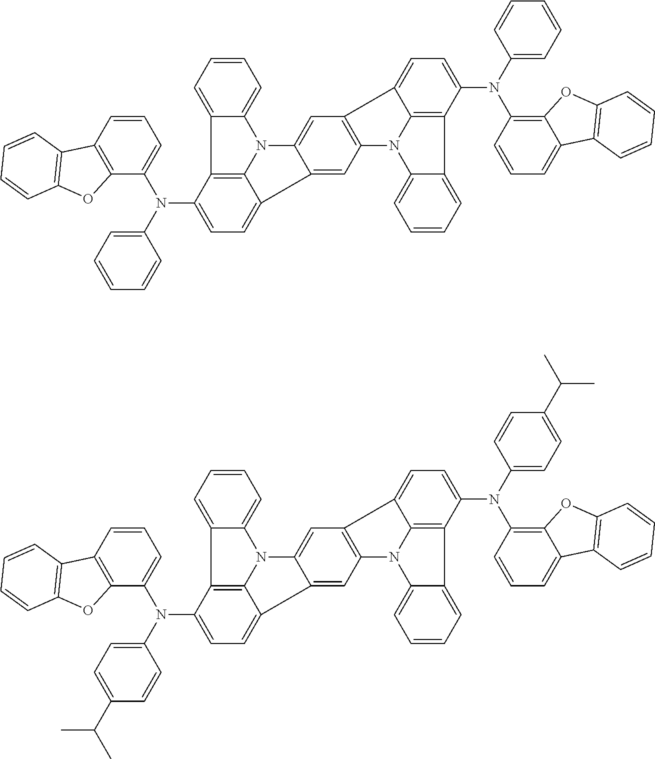 [SMILES: c1ccc(N(c2cccc3c2oc2ccccc23)c2ccc3c4cc5c(cc4n4c6ccccc6c2c34)c2ccc(N(c3ccccc3)c3cccc4c3oc3ccccc34)c3c4ccccc4n5c23)cc1 ; CC(C)c1ccc(N(c2cccc3c2oc2ccccc23)c2ccc3c4cc5c(cc4n4c6ccccc6c2c34)c2ccc(N(c3ccc(C(C)C)cc3)c3cccc4c3oc3ccccc34)c3c4ccccc4n5c23)cc1]

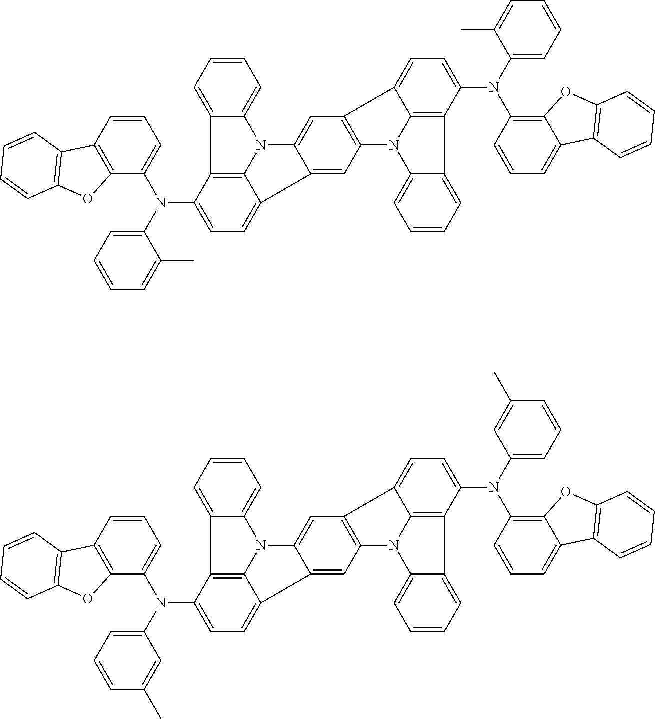 [SMILES: Cc1ccccc1N(c1cccc2c1oc1ccccc12)c1ccc2c3cc4c(cc3n3c5ccccc5c1c23)c1ccc(N(c2ccccc2C)c2cccc3c2oc2ccccc23)c2c3ccccc3n4c12 ; Cc1cccc(N(c2cccc3c2oc2ccccc23)c2ccc3c4cc5c(cc4n4c6ccccc6c2c34)c2ccc(N(c3cccc(C)c3)c3cccc4c3oc3ccccc34)c3c4ccccc4n5c23)c1]

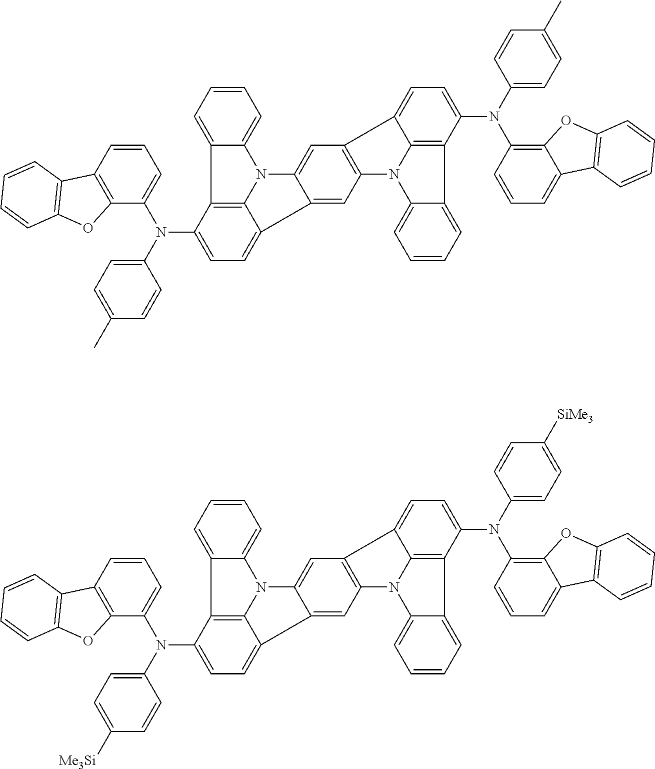 [SMILES: Cc1ccc(N(c2cccc3c2oc2ccccc23)c2ccc3c4cc5c(cc4n4c6ccccc6c2c34)c2ccc(N(c3ccc(C)cc3)c3cccc4c3oc3ccccc34)c3c4ccccc4n5c23)cc1 ; C[Si](C)(C)c1ccc(N(c2cccc3c2oc2ccccc23)c2ccc3c4cc5c(cc4n4c6ccccc6c2c34)c2ccc(N(c3ccc([Si](C)(C)C)cc3)c3cccc4c3oc3ccccc34)c3c4ccccc4n5c23)cc1]

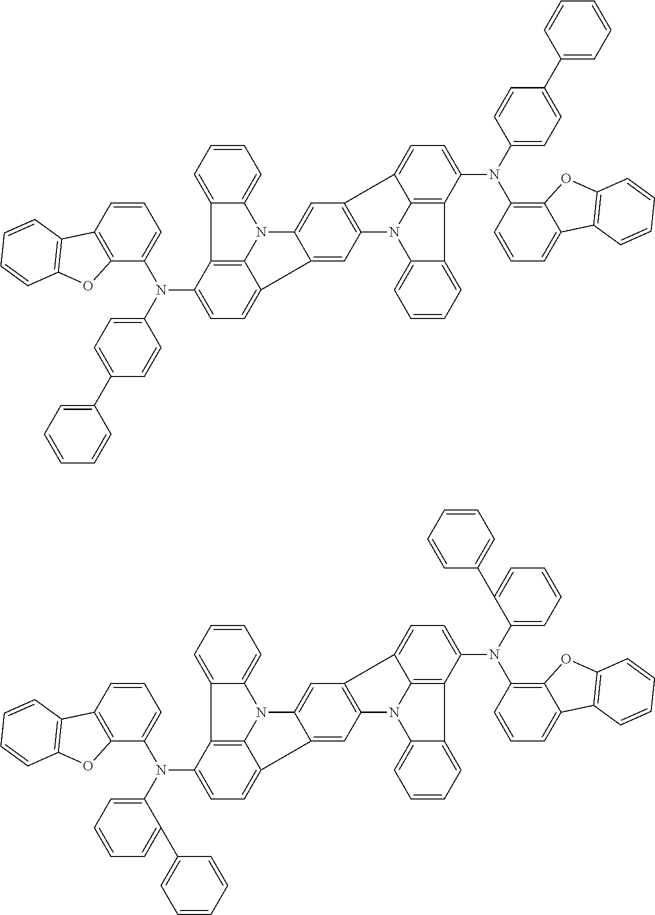 [SMILES: c1ccc(-c2ccc(N(c3cccc4c3oc3ccccc34)c3ccc4c5cc6c(cc5n5c7ccccc7c3c45)c3ccc(N(c4ccc(-c5ccccc5)cc4)c4cccc5c4oc4ccccc45)c4c5ccccc5n6c34)cc2)cc1 ; c1ccc(-c2ccccc2N(c2cccc3c2oc2ccccc23)c2ccc3c4cc5c(cc4n4c6ccccc6c2c34)c2ccc(N(c3ccccc3-c3ccccc3)c3cccc4c3oc3ccccc34)c3c4ccccc4n5c23)cc1]

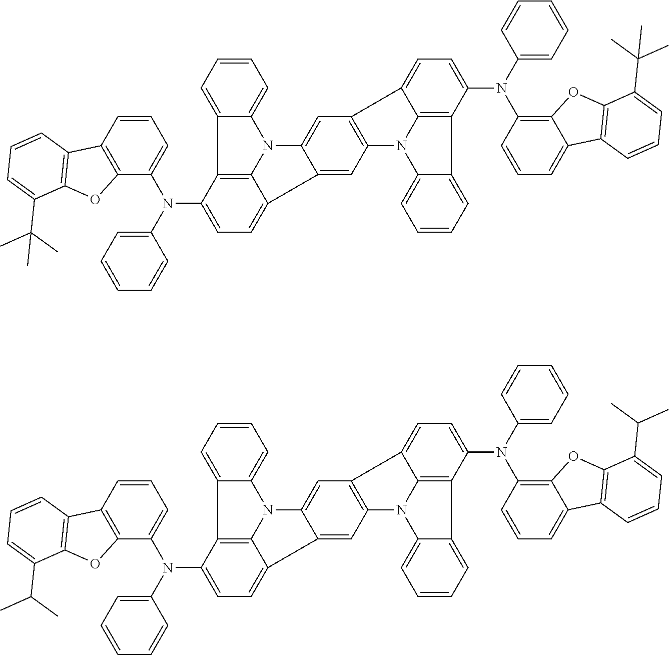

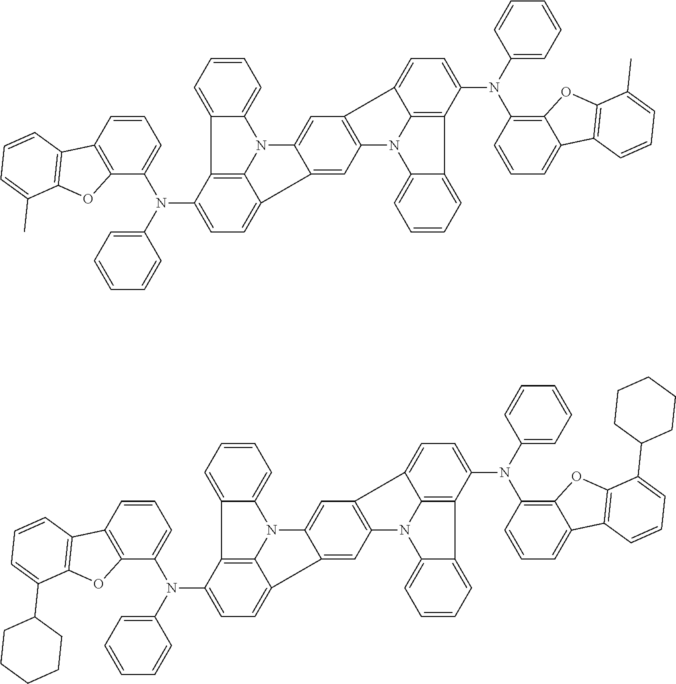 [SMILES: Cc1cccc2c1oc1c(N(c3ccccc3)c3ccc4c5cc6c(cc5n5c7ccccc7c3c45)c3ccc(N(c4ccccc4)c4cccc5c4oc4c(C)cccc45)c4c5ccccc5n6c34)cccc12 ; c1ccc(N(c2cccc3c2oc2c(C4CCCCC4)cccc23)c2ccc3c4cc5c(cc4n4c6ccccc6c2c34)c2ccc(N(c3ccccc3)c3cccc4c3oc3c(C6CCCCC6)cccc34)c3c4ccccc4n5c23)cc1]

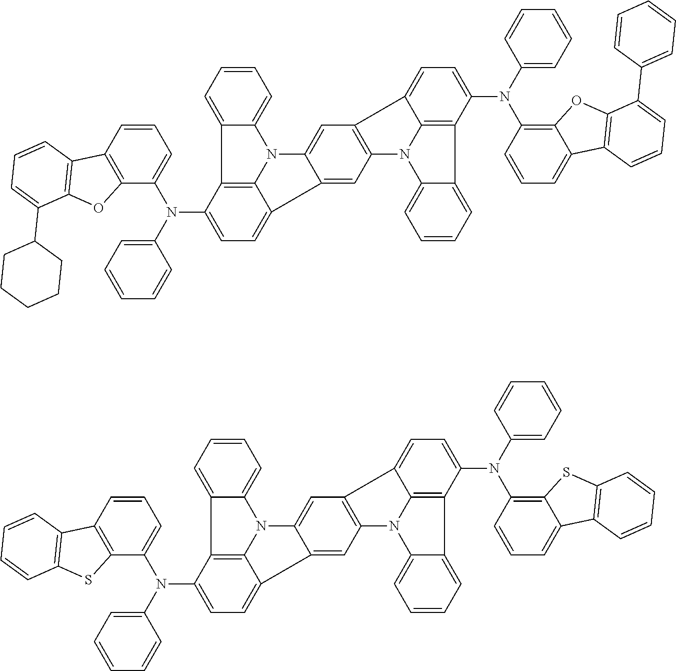 [SMILES: c1ccc(-c2cccc3c2oc2c(N(c4ccccc4)c4ccc5c6cc7c(cc6n6c8ccccc8c4c56)c4ccc(N(c5ccccc5)c5cccc6c5oc5c(C8CCCCC8)cccc56)c5c6ccccc6n7c45)cccc23)cc1 ; c1ccc(N(c2cccc3c2sc2ccccc23)c2ccc3c4cc5c(cc4n4c6ccccc6c2c34)c2ccc(N(c3ccccc3)c3cccc4c3sc3ccccc34)c3c4ccccc4n5c23)cc1]

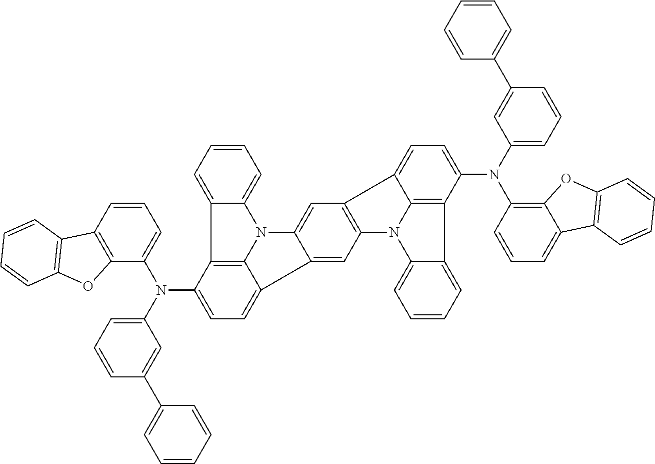 [SMILES: c1ccc(-c2cccc(N(c3cccc4c3oc3ccccc34)c3ccc4c5cc6c(cc5n5c7ccccc7c3c45)c3ccc(N(c4cccc(-c5ccccc5)c4)c4cccc5c4oc4ccccc45)c4c5ccccc5n6c34)c2)cc1]

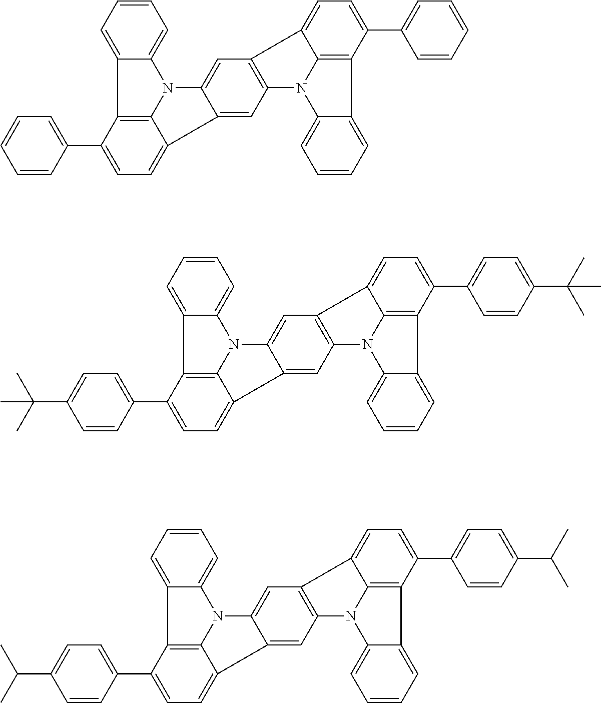

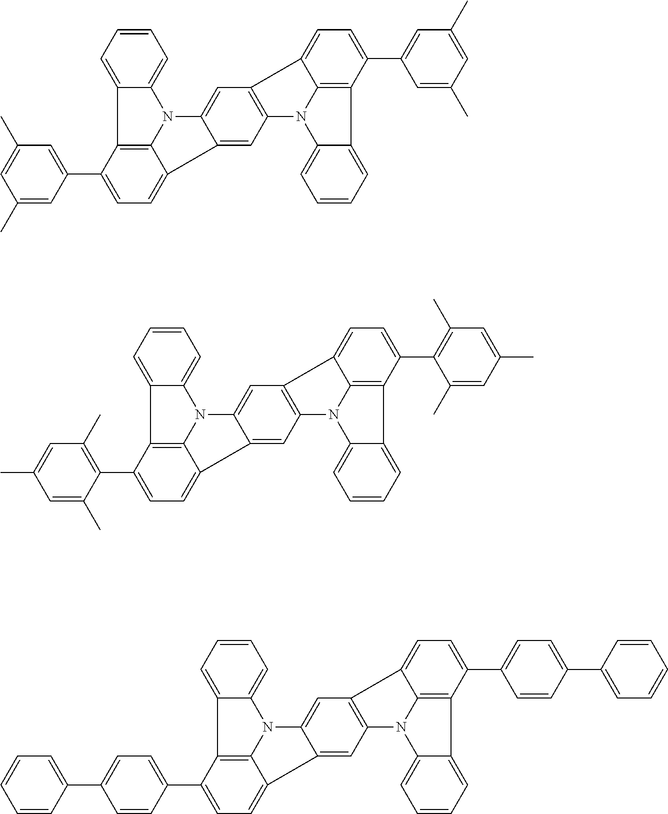

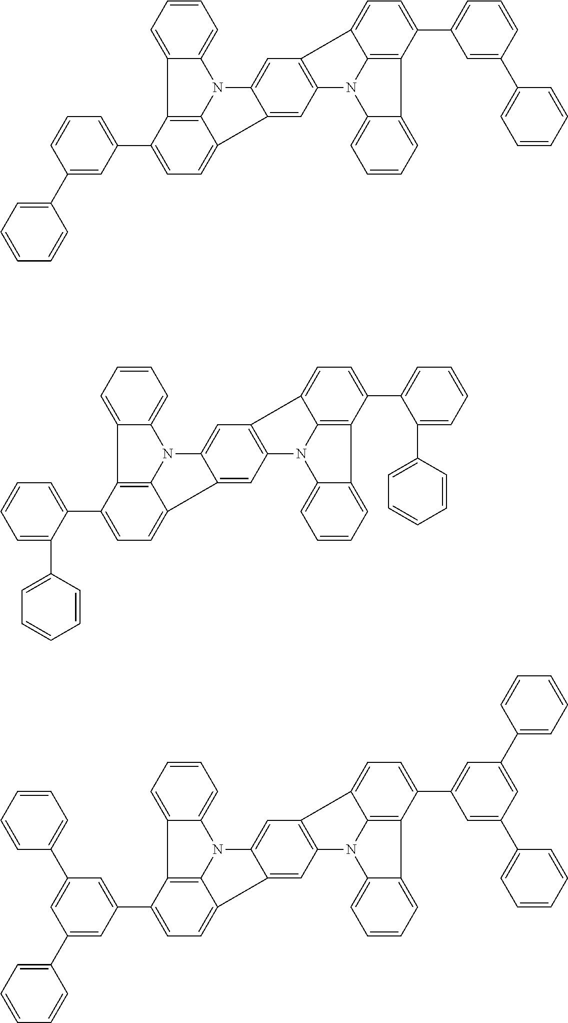 [SMILES: c1ccc(-c2cccc(-c3ccc4c5cc6c(cc5n5c7ccccc7c3c45)c3ccc(-c4cccc(-c5ccccc5)c4)c4c5ccccc5n6c34)c2)cc1 ; c1ccc(-c2ccccc2-c2ccc3c4cc5c(cc4n4c6ccccc6c2c34)c2ccc(-c3ccccc3-c3ccccc3)c3c4ccccc4n5c23)cc1 ; c1ccc(-c2cc(-c3ccccc3)cc(-c3ccc4c5cc6c(cc5n5c7ccccc7c3c45)c3ccc(-c4cc(-c5ccccc5)cc(-c5ccccc5)c4)c4c5ccccc5n6c34)c2)cc1]

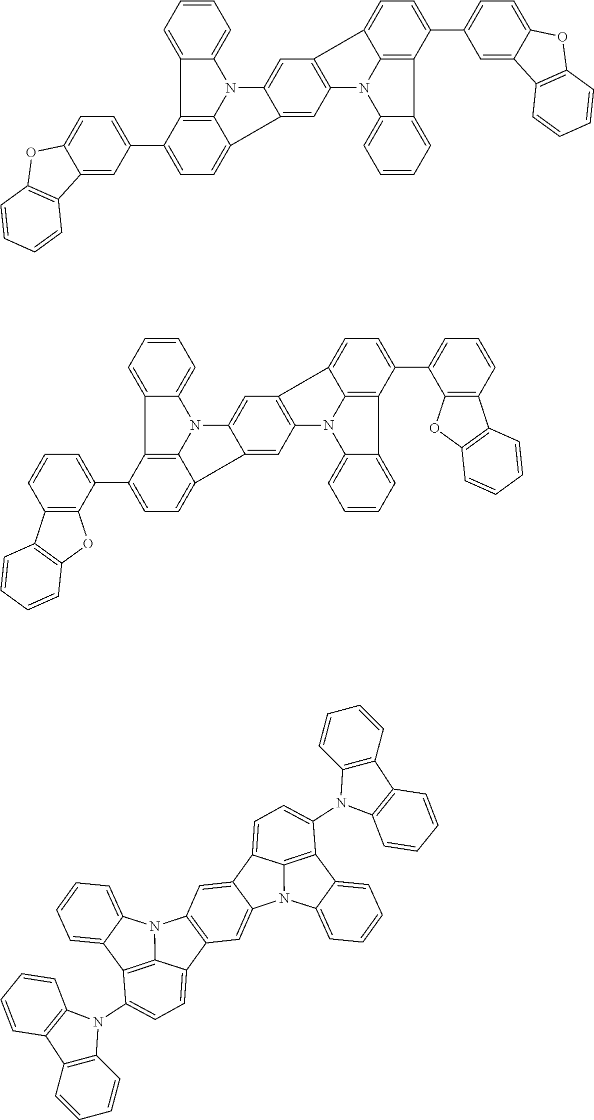 [SMILES: c1ccc2c(c1)oc1ccc(-c3ccc4c5cc6c(cc5n5c7ccccc7c3c45)c3ccc(-c4ccc5oc7ccccc7c5c4)c4c5ccccc5n6c34)cc12 ; c1ccc2c(c1)oc1c(-c3ccc4c5cc6c(cc5n5c7ccccc7c3c45)c3ccc(-c4cccc5c4oc4ccccc45)c4c5ccccc5n6c34)cccc12 ; c1ccc2c(c1)c1ccccc1n2-c1ccc2c3cc4c(cc3n3c5ccccc5c1c23)c1ccc(-n2c3ccccc3c3ccccc32)c2c3ccccc3n4c12]

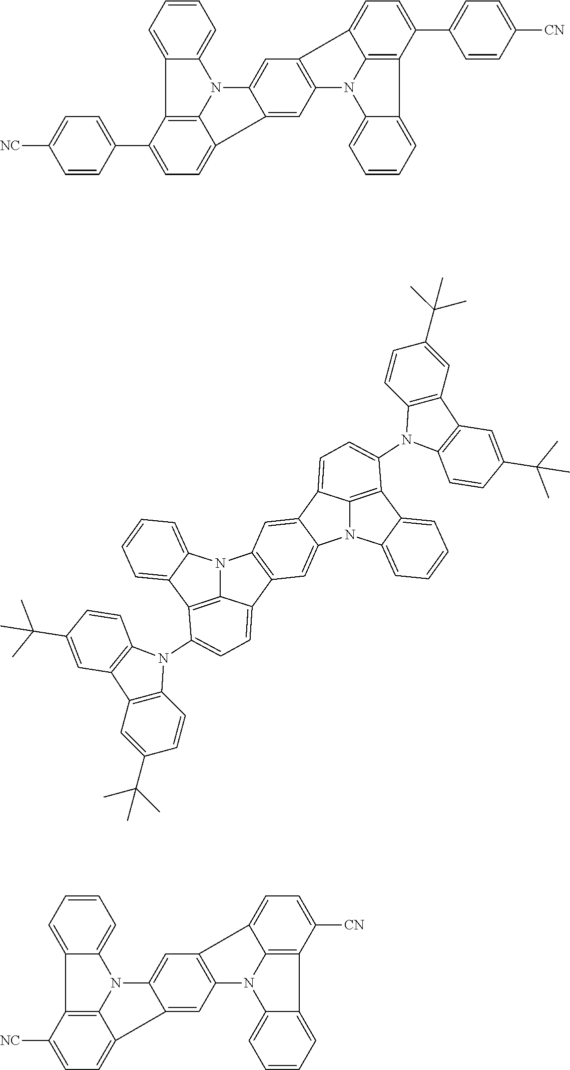 [SMILES: N#Cc1ccc(-c2ccc3c4cc5c(cc4n4c6ccccc6c2c34)c2ccc(-c3ccc(C#N)cc3)c3c4ccccc4n5c23)cc1 ; CC(C)(C)c1ccc2c(c1)c1cc(C(C)(C)C)ccc1n2-c1ccc2c3cc4c(cc3n3c5ccccc5c1c23)c1ccc(-n2c3ccc(C(C)(C)C)cc3c3cc(C(C)(C)C)ccc32)c2c3ccccc3n4c12 ; N#Cc1ccc2c3cc4c(cc3n3c5ccccc5c1c23)c1ccc(C#N)c2c3ccccc3n4c12]

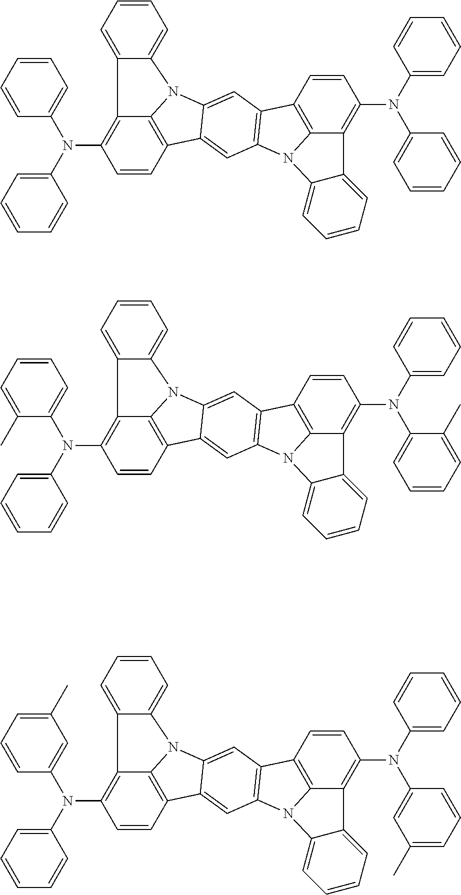 [SMILES: c1ccc(N(c2ccccc2)c2ccc3c4cc5c(cc4n4c6ccccc6c2c34)c2ccc(N(c3ccccc3)c3ccccc3)c3c4ccccc4n5c23)cc1 ; Cc1ccccc1N(c1ccccc1)c1ccc2c3cc4c(cc3n3c5ccccc5c1c23)c1ccc(N(c2ccccc2)c2ccccc2C)c2c3ccccc3n4c12 ; Cc1cccc(N(c2ccccc2)c2ccc3c4cc5c(cc4n4c6ccccc6c2c34)c2ccc(N(c3ccccc3)c3cccc(C)c3)c3c4ccccc4n5c23)c1]

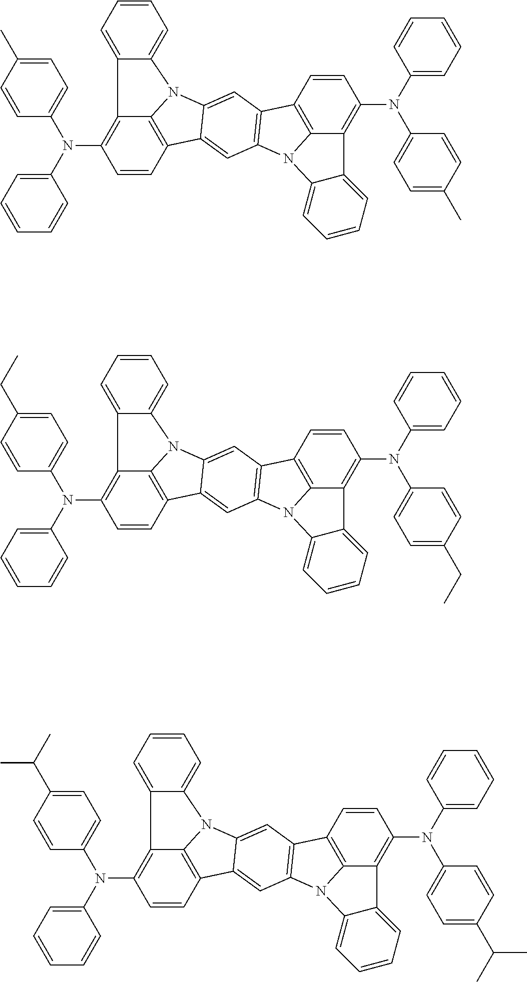

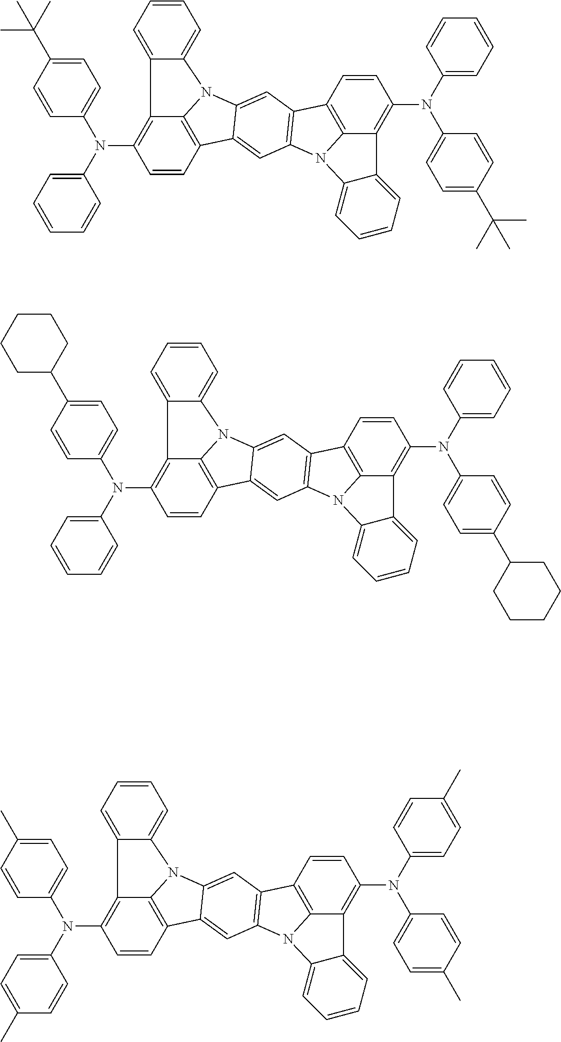 [SMILES: CC(C)(C)c1ccc(N(c2ccccc2)c2ccc3c4cc5c(cc4n4c6ccccc6c2c34)c2ccc(N(c3ccccc3)c3ccc(C(C)(C)C)cc3)c3c4ccccc4n5c23)cc1 ; c1ccc(N(c2ccc(C3CCCCC3)cc2)c2ccc3c4cc5c(cc4n4c6ccccc6c2c34)c2ccc(N(c3ccccc3)c3ccc(C4CCCCC4)cc3)c3c4ccccc4n5c23)cc1 ; Cc1ccc(N(c2ccc(C)cc2)c2ccc3c4cc5c(cc4n4c6ccccc6c2c34)c2ccc(N(c3ccc(C)cc3)c3ccc(C)cc3)c3c4ccccc4n5c23)cc1]

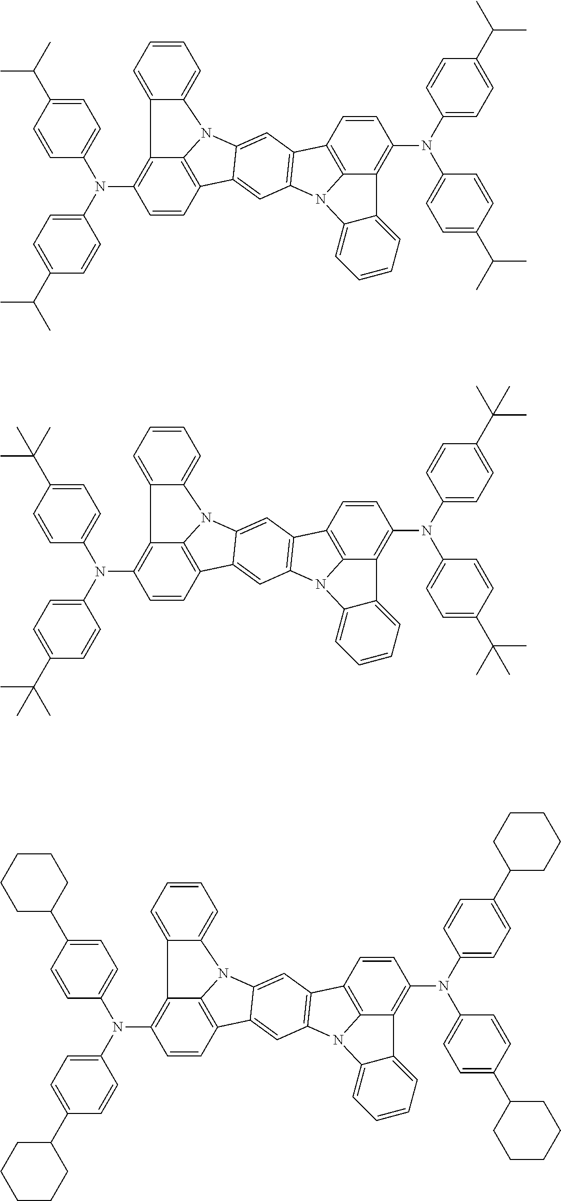

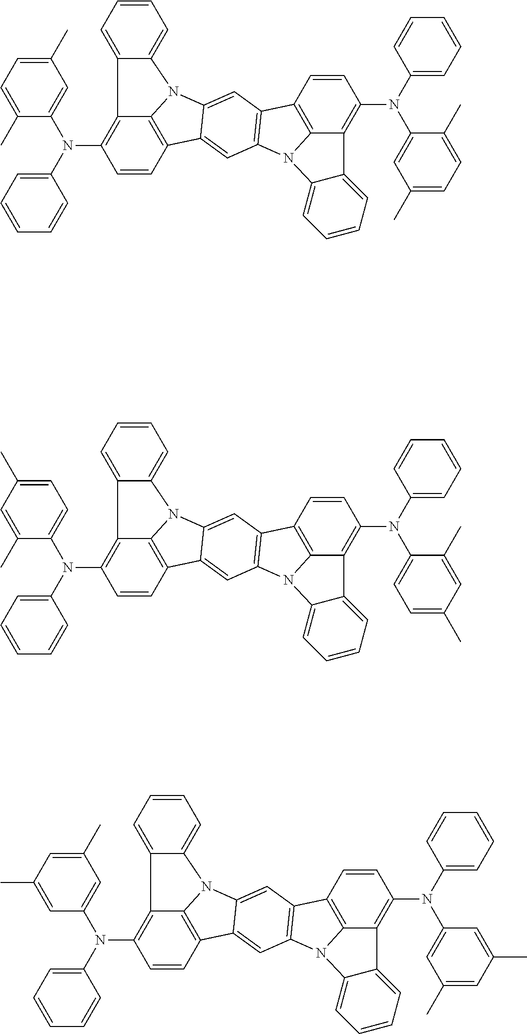

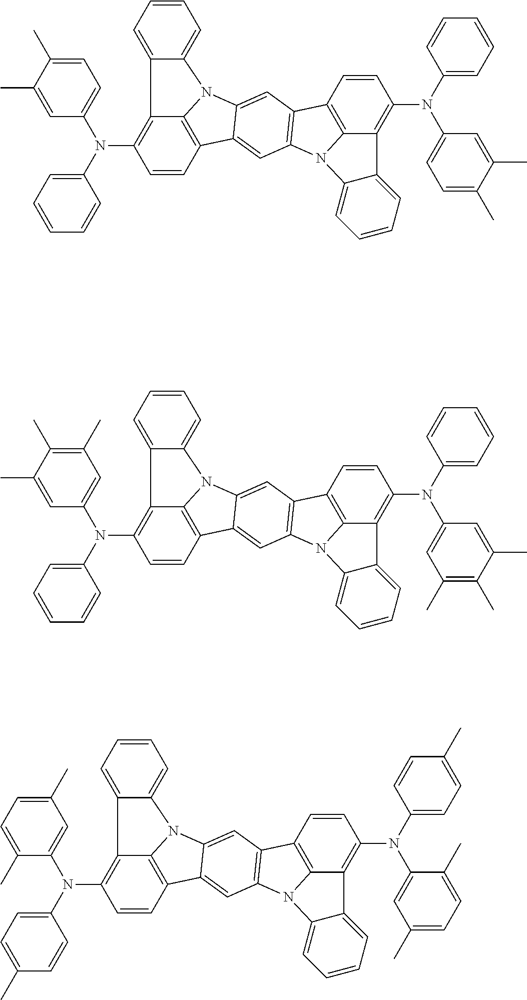

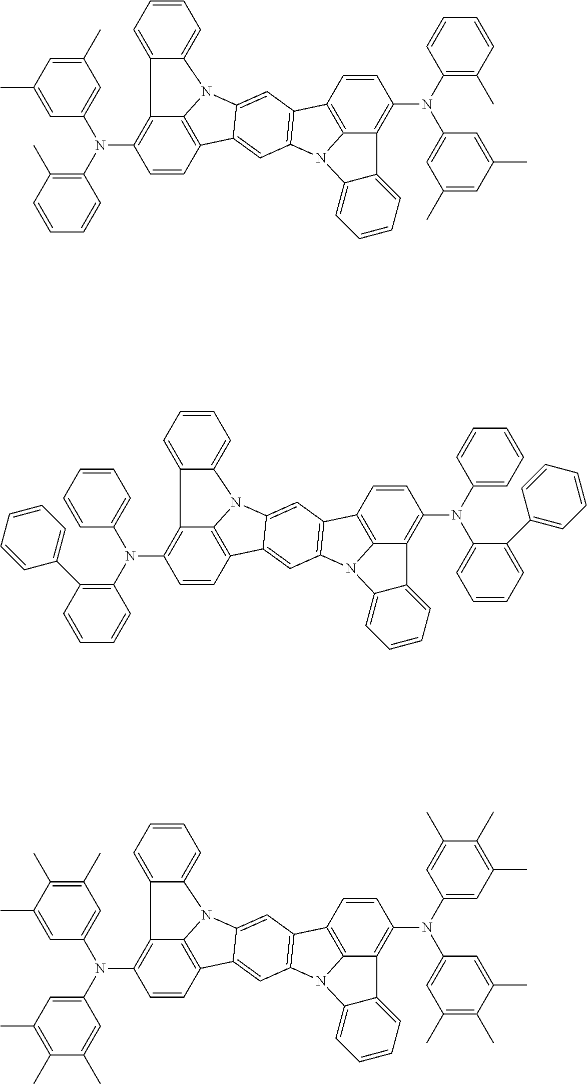

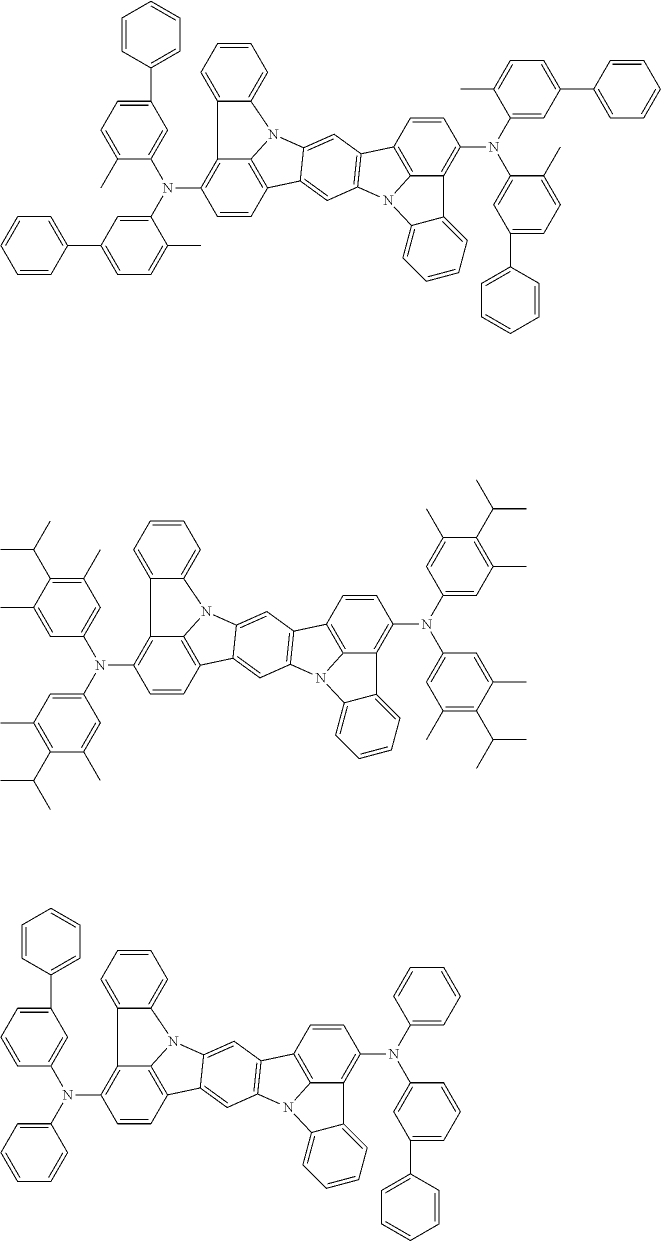 [SMILES: Cc1ccc(-c2ccccc2)cc1N(c1cc(-c2ccccc2)ccc1C)c1ccc2c3cc4c(cc3n3c5ccccc5c1c23)c1ccc(N(c2cc(-c3ccccc3)ccc2C)c2cc(-c3ccccc3)ccc2C)c2c3ccccc3n4c12 ; Cc1cc(N(c2cc(C)c(C(C)C)c(C)c2)c2ccc3c4cc5c(cc4n4c6ccccc6c2c34)c2ccc(N(c3cc(C)c(C(C)C)c(C)c3)c3cc(C)c(C(C)C)c(C)c3)c3c4ccccc4n5c23)cc(C)c1C(C)C ; c1ccc(-c2cccc(N(c3ccccc3)c3ccc4c5cc6c(cc5n5c7ccccc7c3c45)c3ccc(N(c4ccccc4)c4cccc(-c5ccccc5)c4)c4c5ccccc5n6c34)c2)cc1]

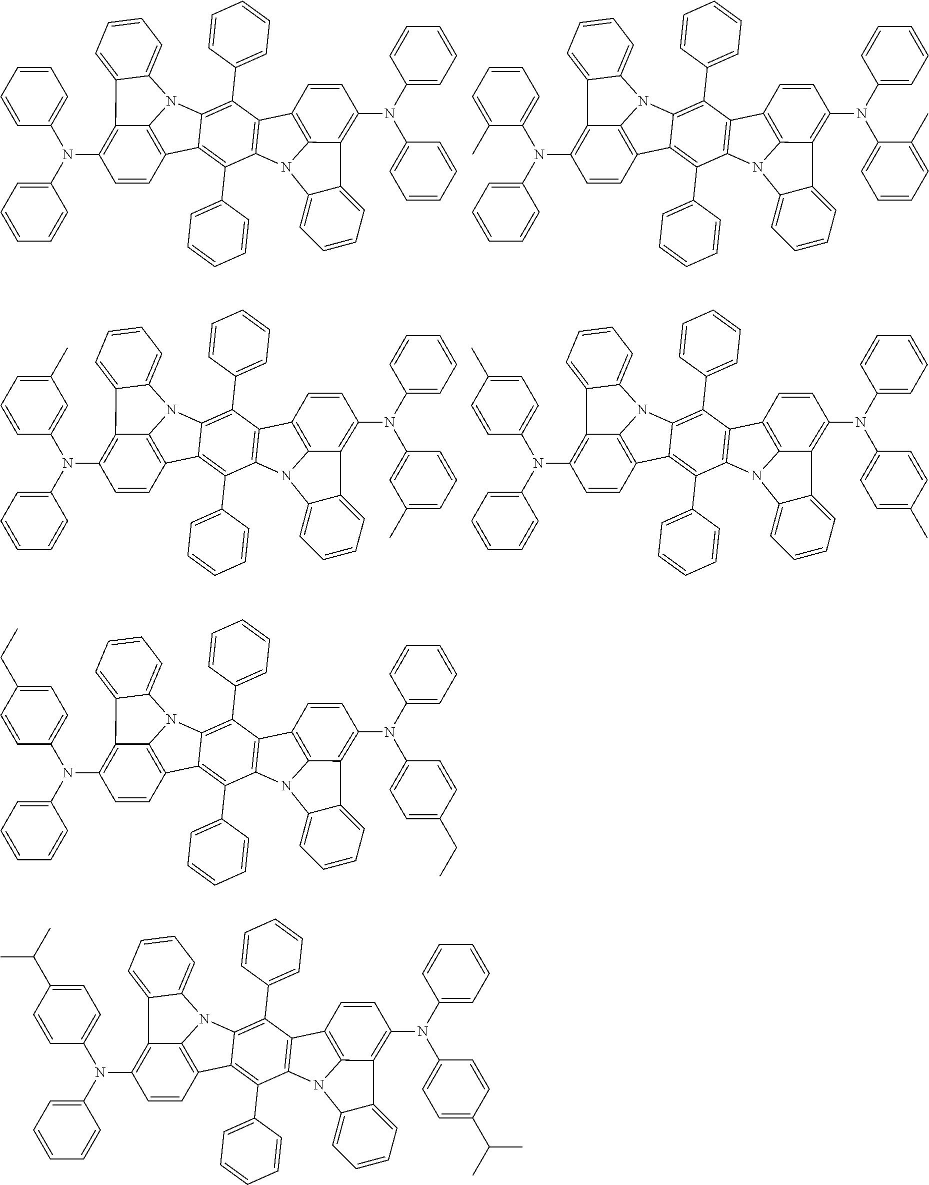

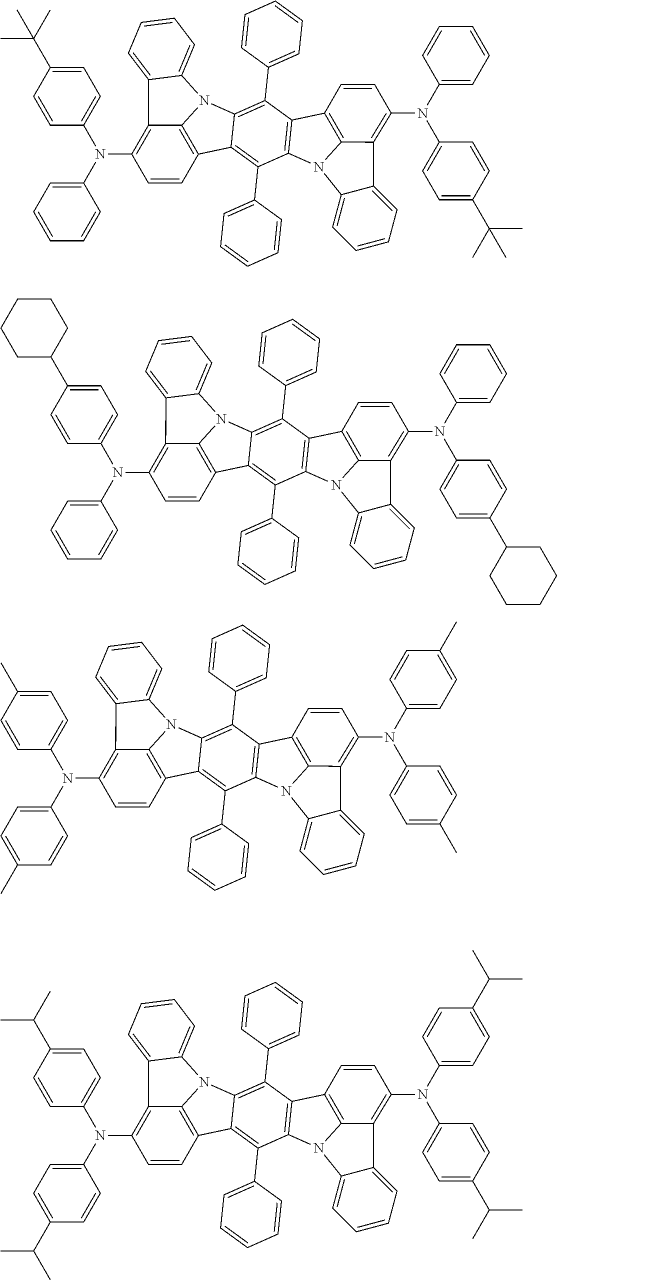

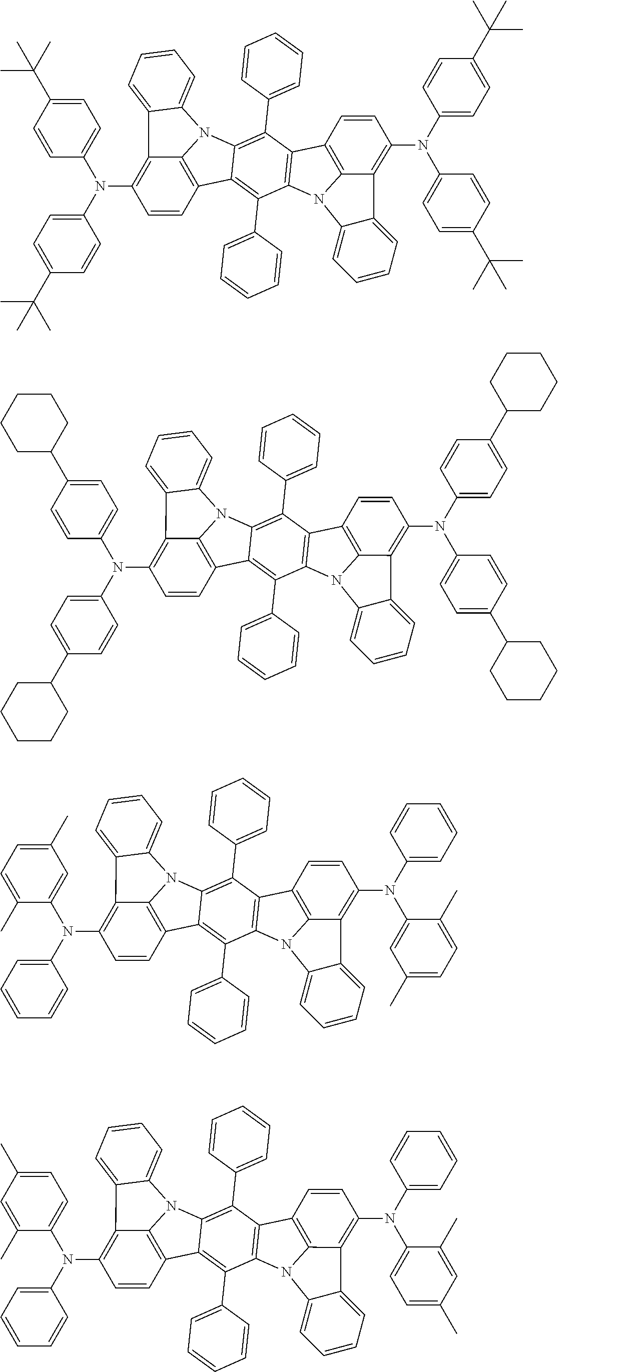

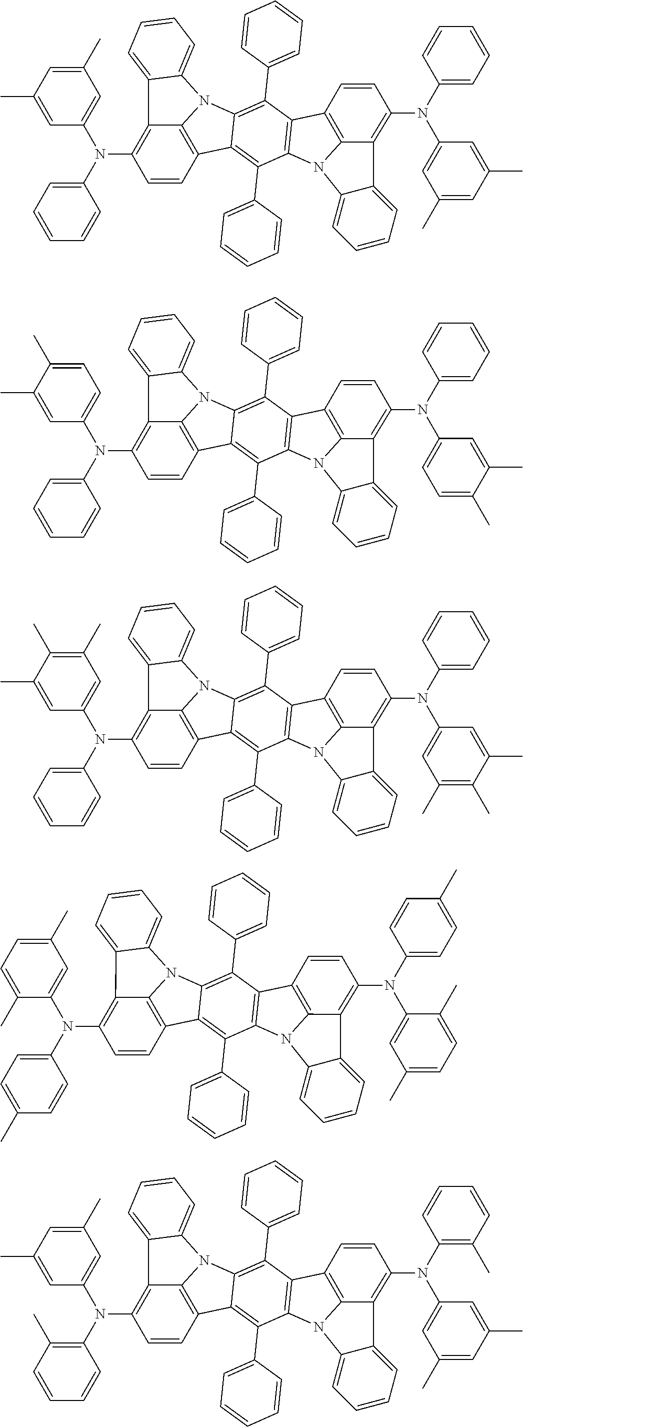

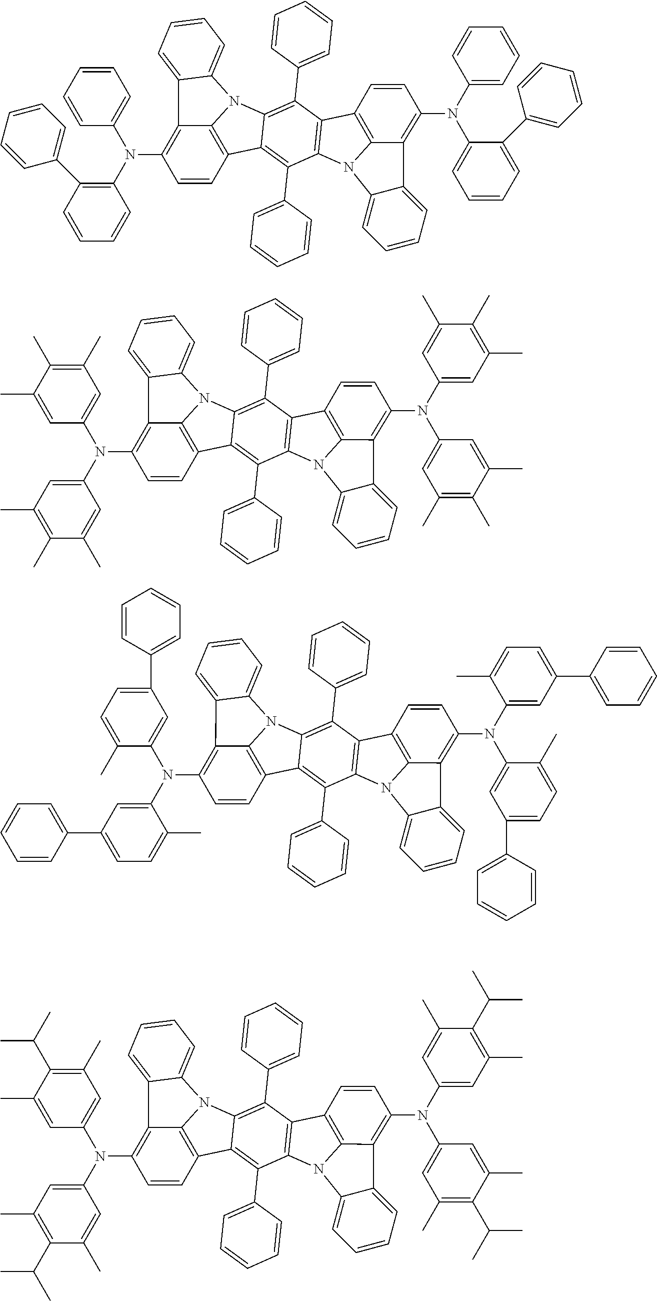

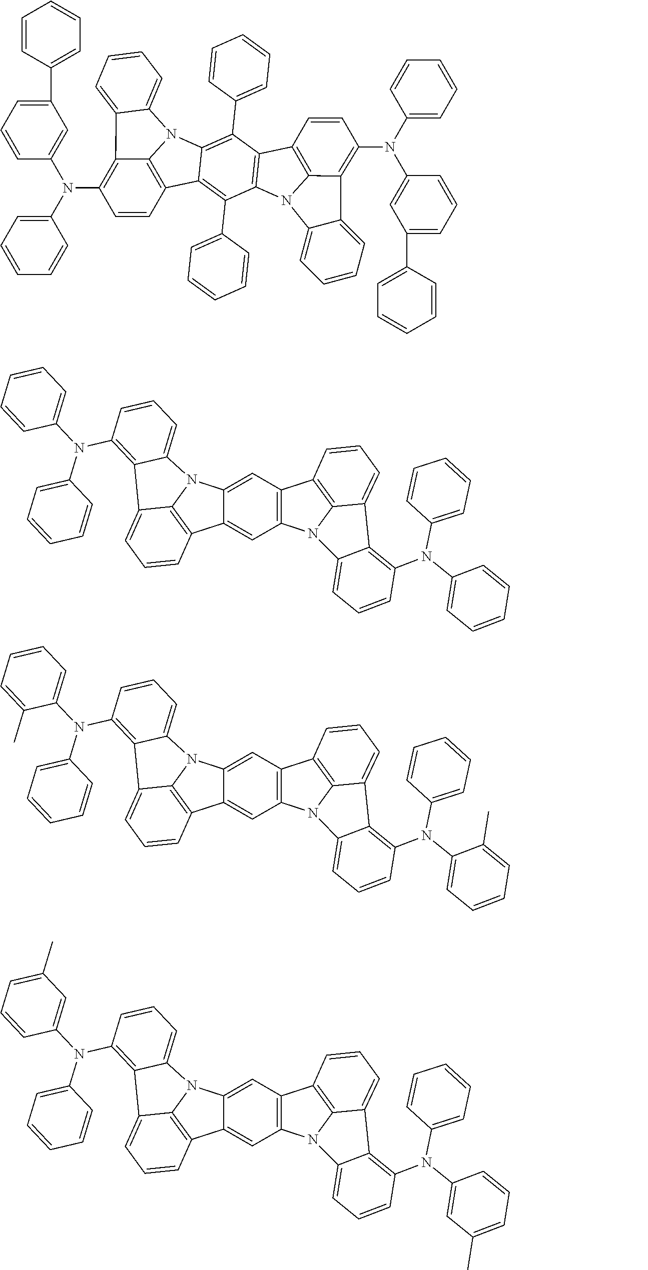 [SMILES: c1ccc(-c2cccc(N(c3ccccc3)c3ccc4c5c(-c6ccccc6)c6c(c(-c7ccccc7)c5n5c7ccccc7c3c45)c3ccc(N(c4ccccc4)c4cccc(-c5ccccc5)c4)c4c5ccccc5n6c34)c2)cc1 ; c1ccc(N(c2ccccc2)c2cccc3c2c2cccc4c5cc6c(cc5n3c42)c2cccc3c4c(N(c5ccccc5)c5ccccc5)cccc4n6c23)cc1 ; Cc1ccccc1N(c1ccccc1)c1cccc2c1c1cccc3c4cc5c(cc4n2c31)c1cccc2c3c(N(c4ccccc4)c4ccccc4C)cccc3n5c12 ; Cc1cccc(N(c2ccccc2)c2cccc3c2c2cccc4c5cc6c(cc5n3c42)c2cccc3c4c(N(c5ccccc5)c5cccc(C)c5)cccc4n6c23)c1]

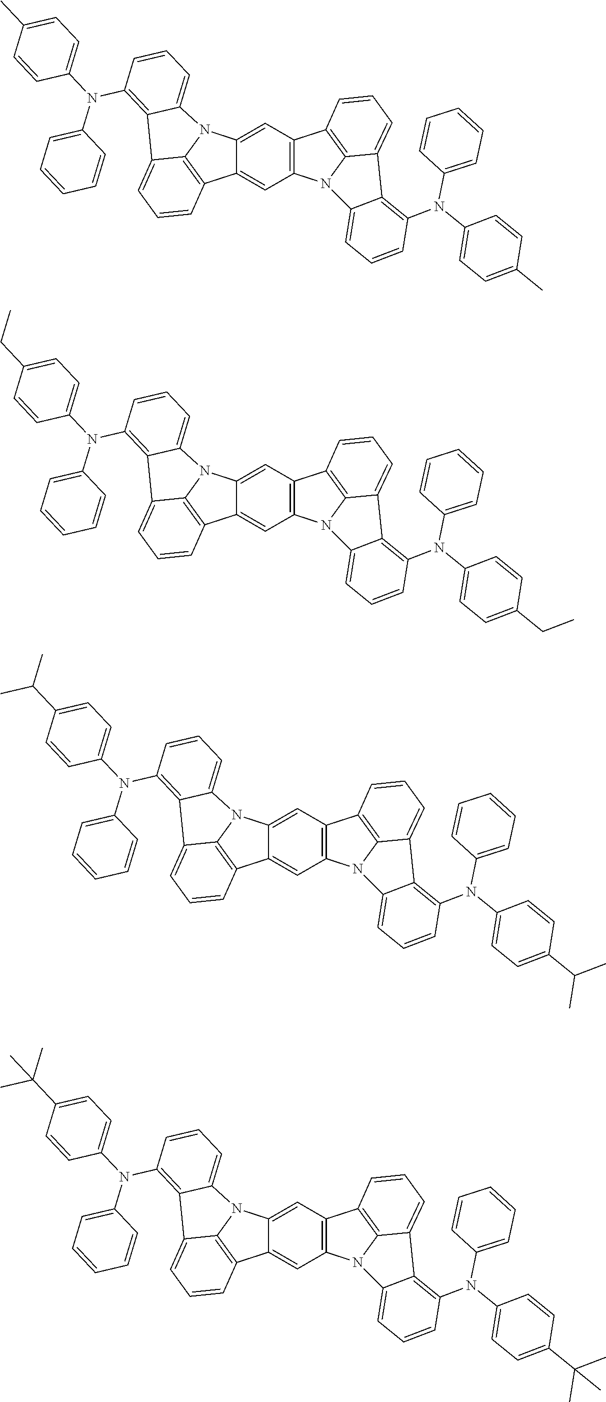

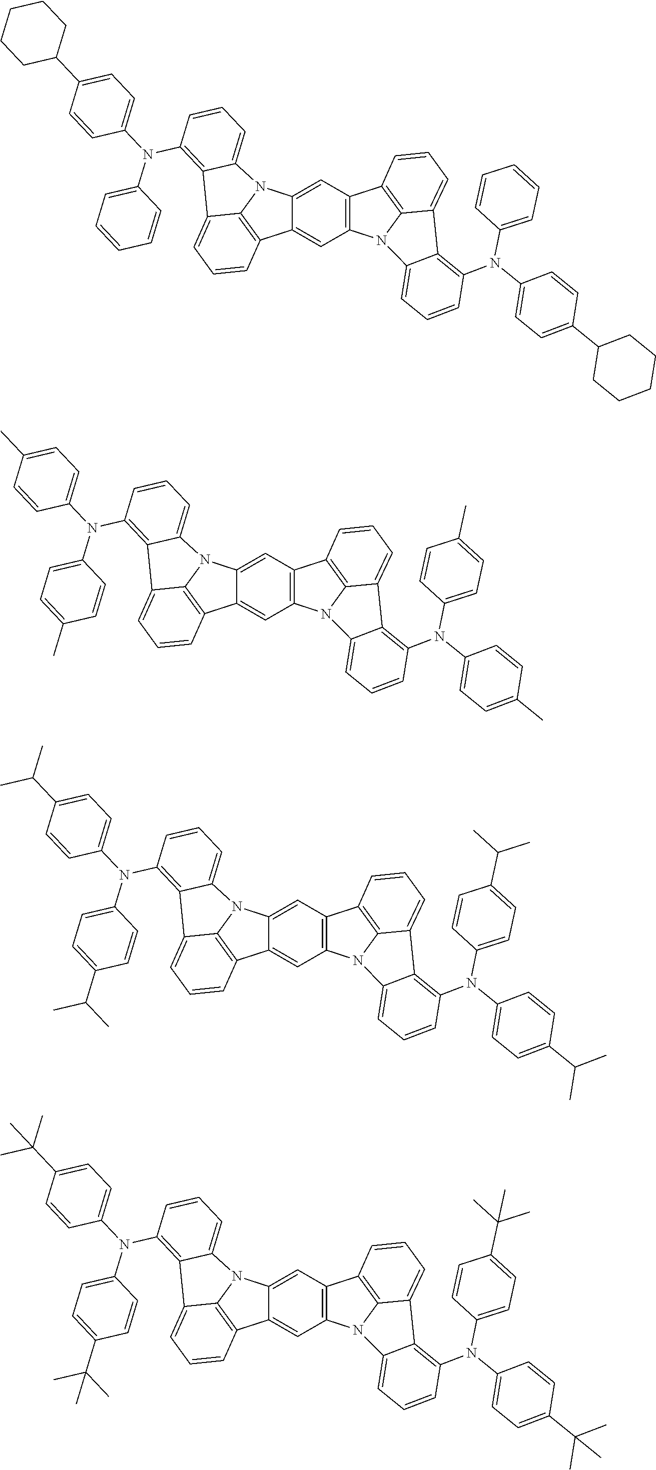

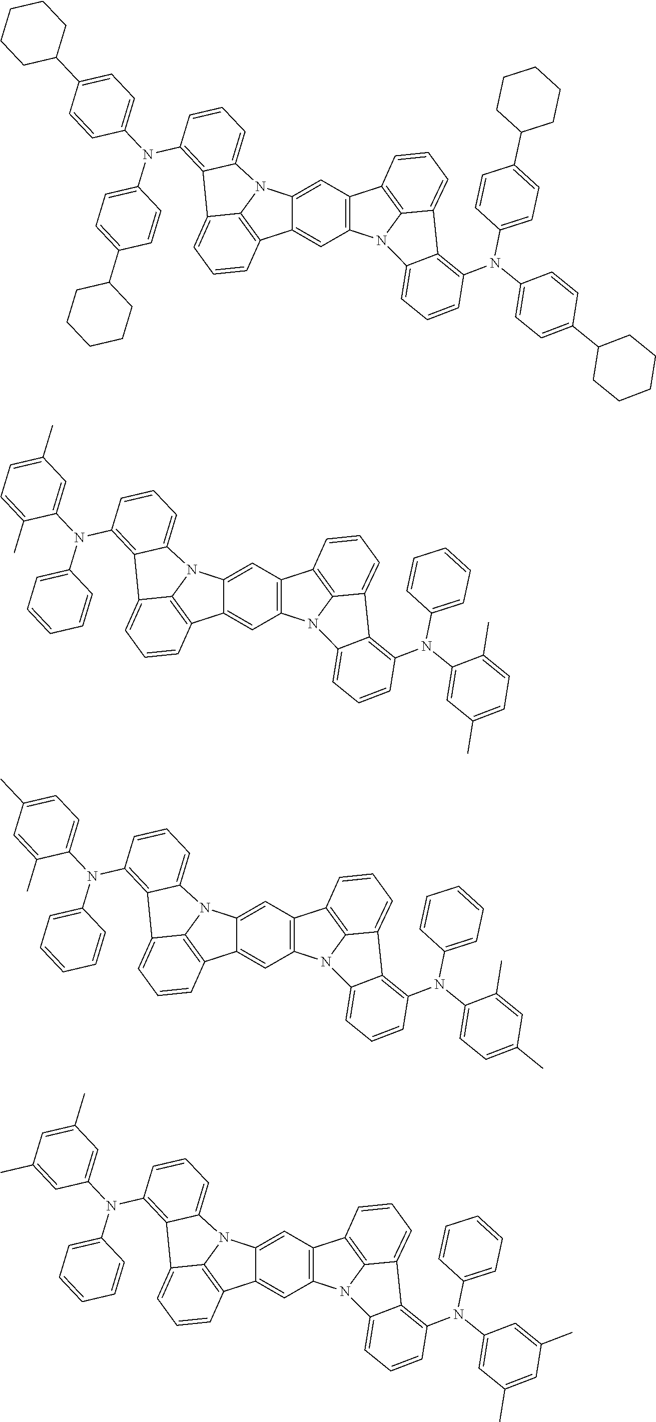

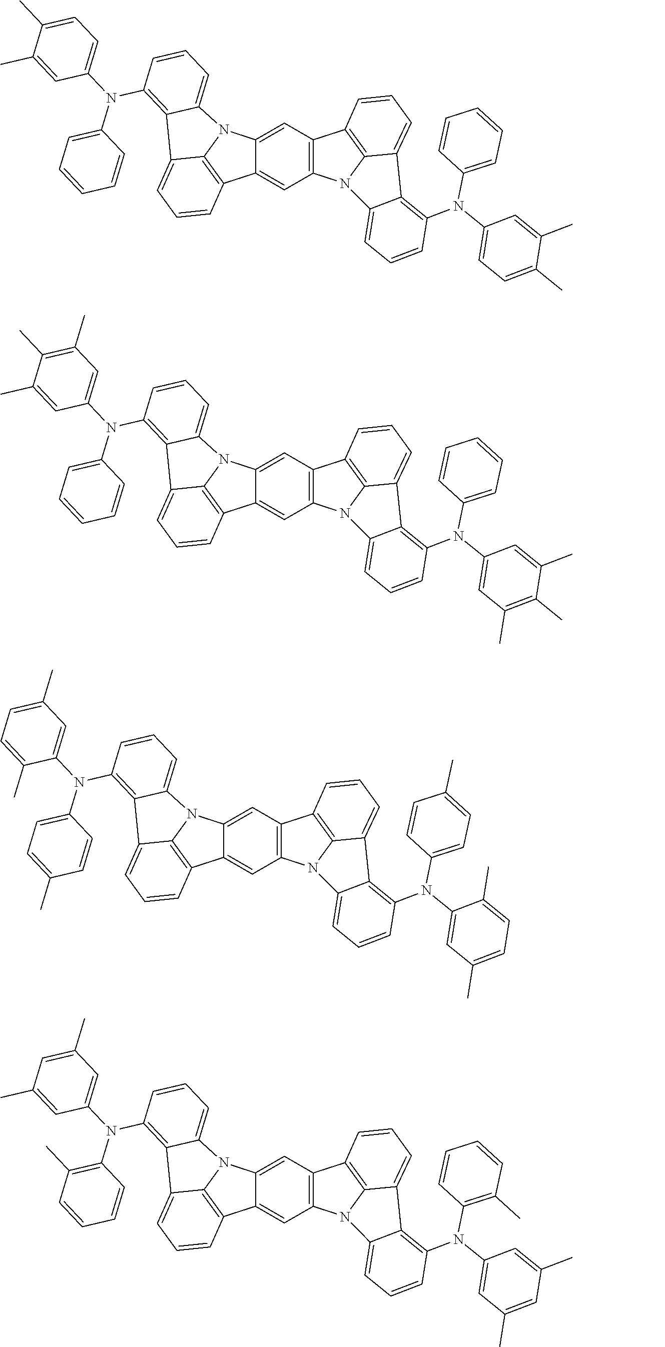

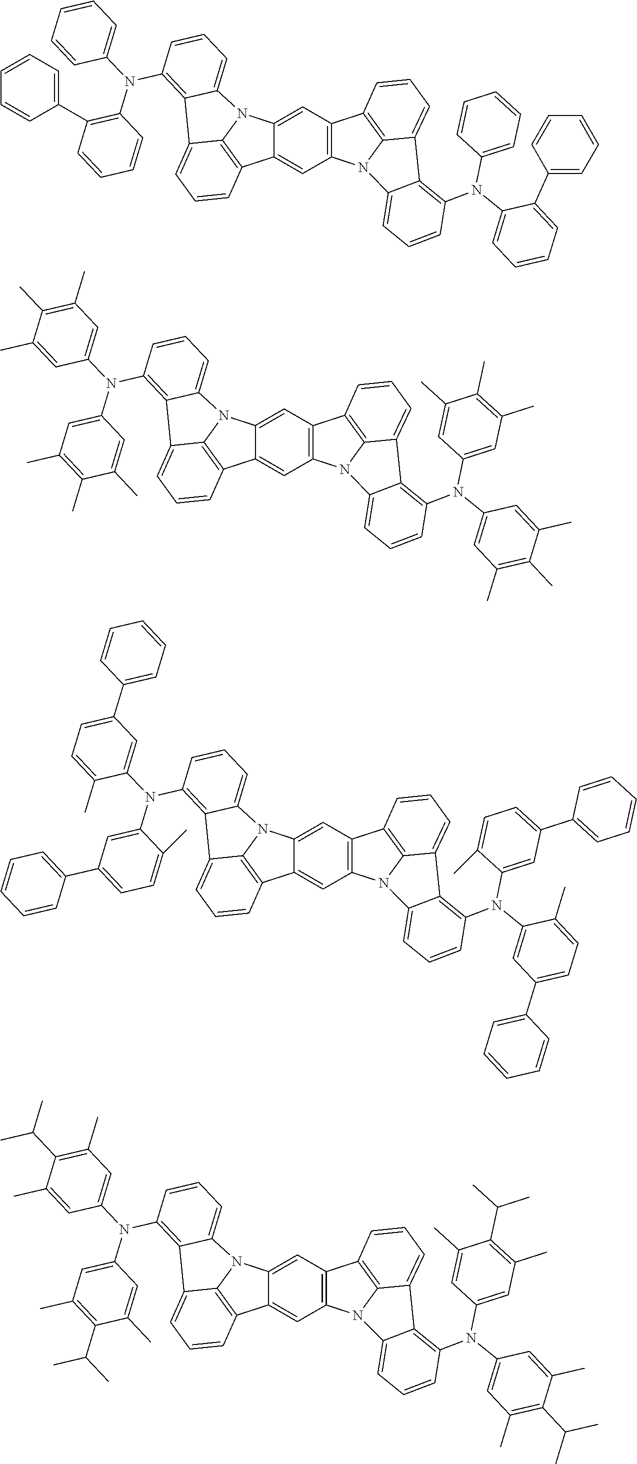

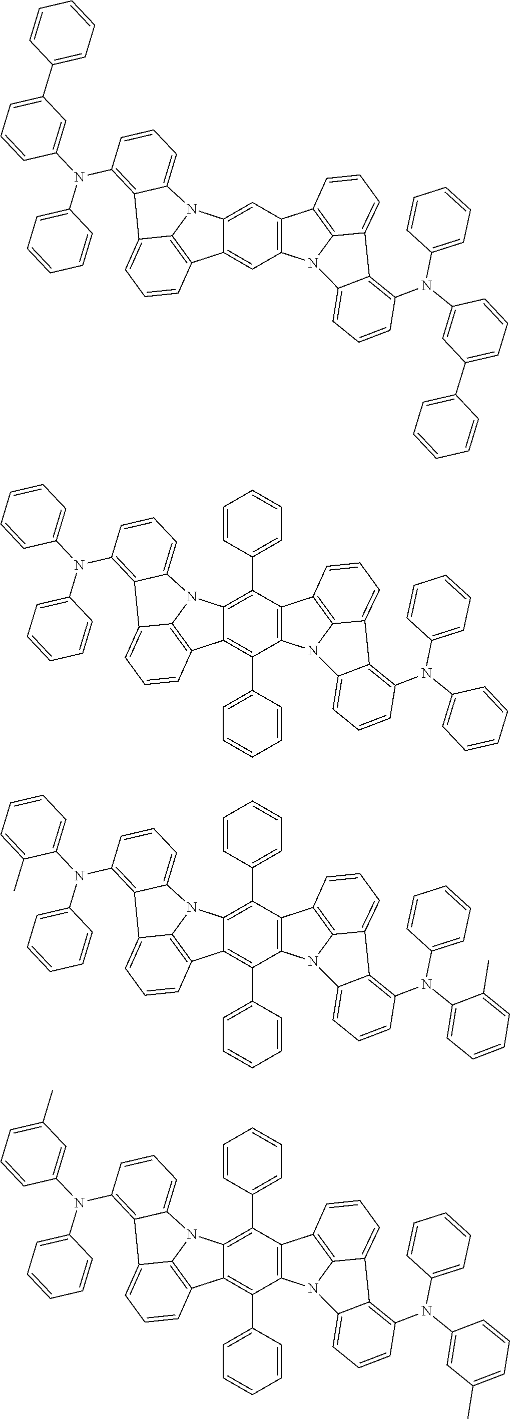

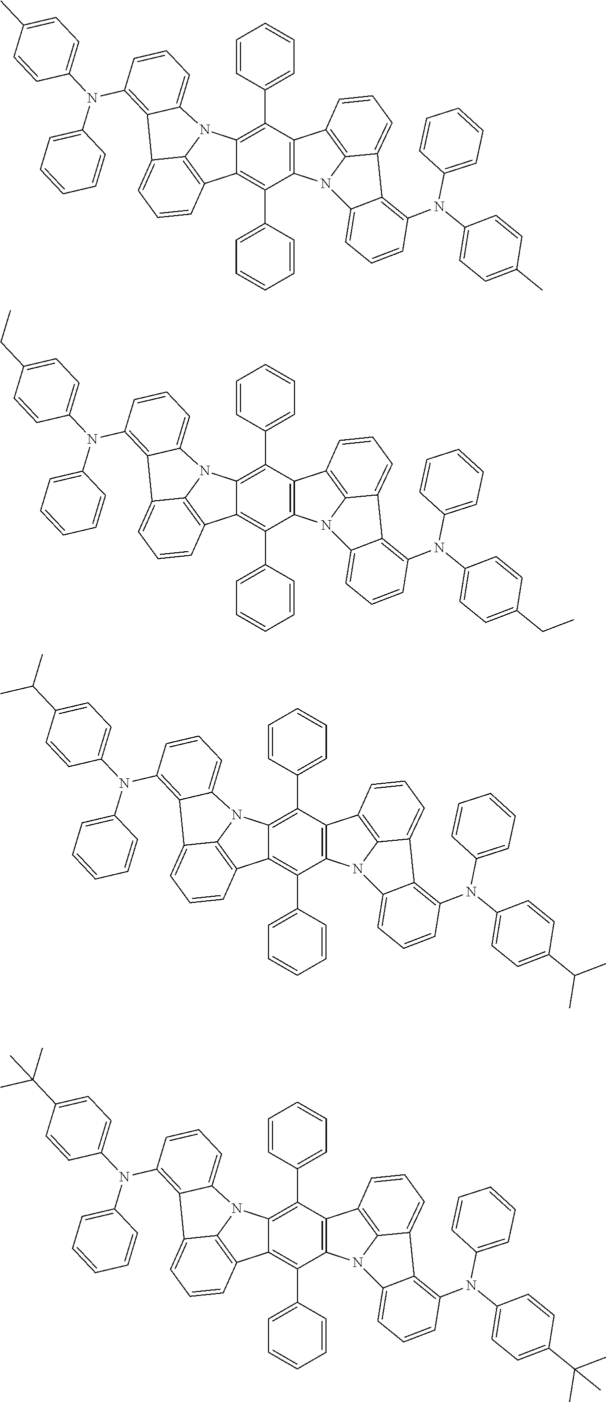

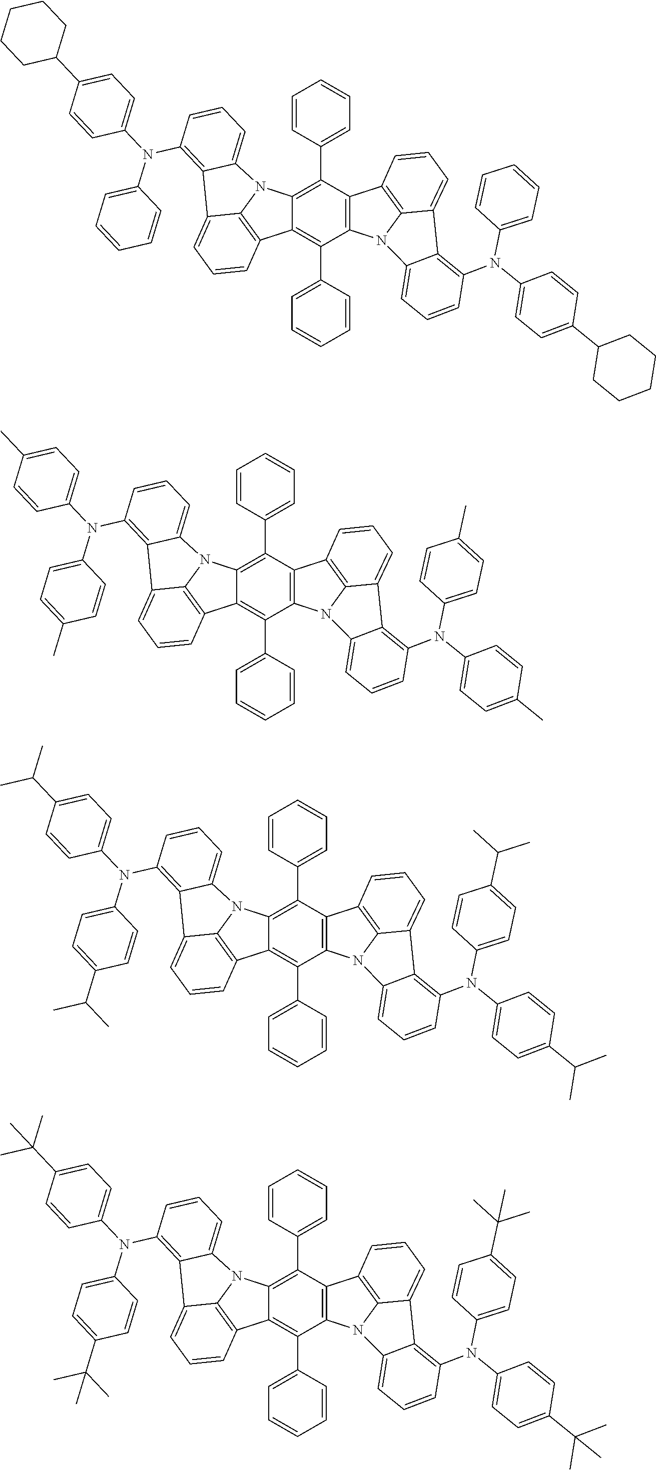

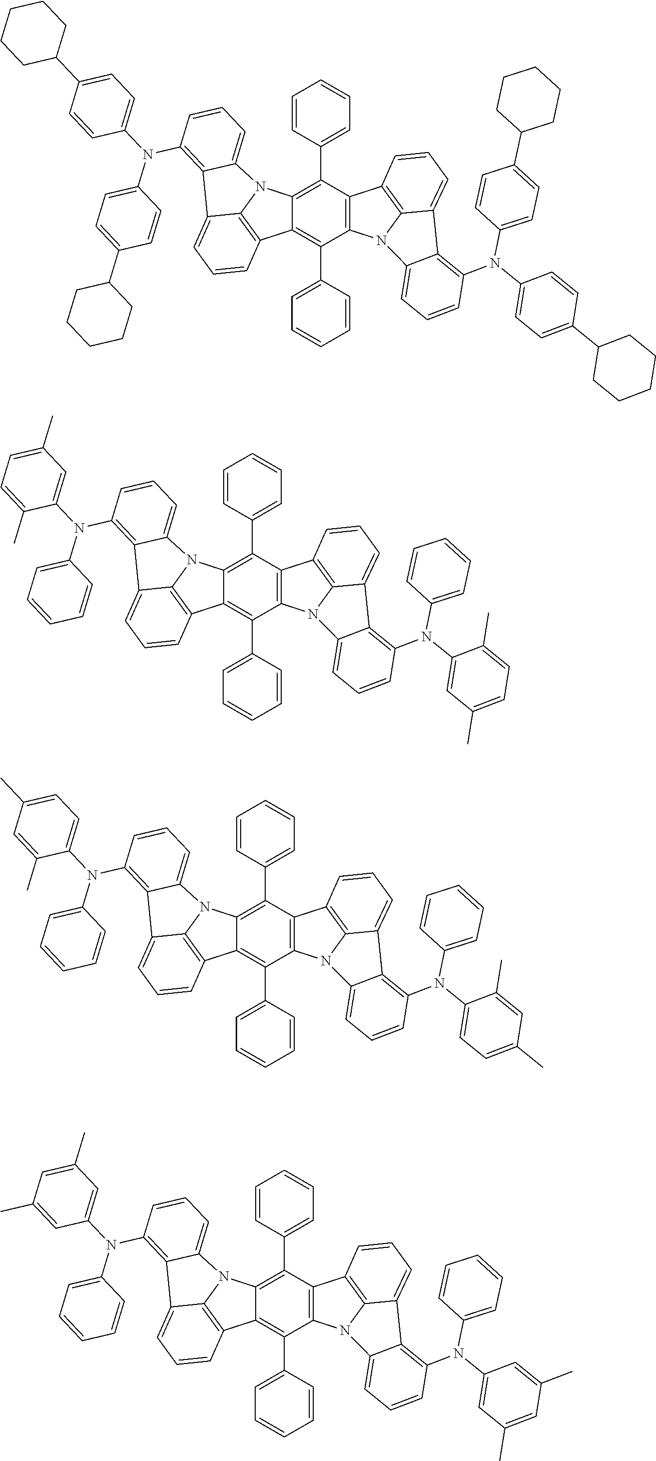

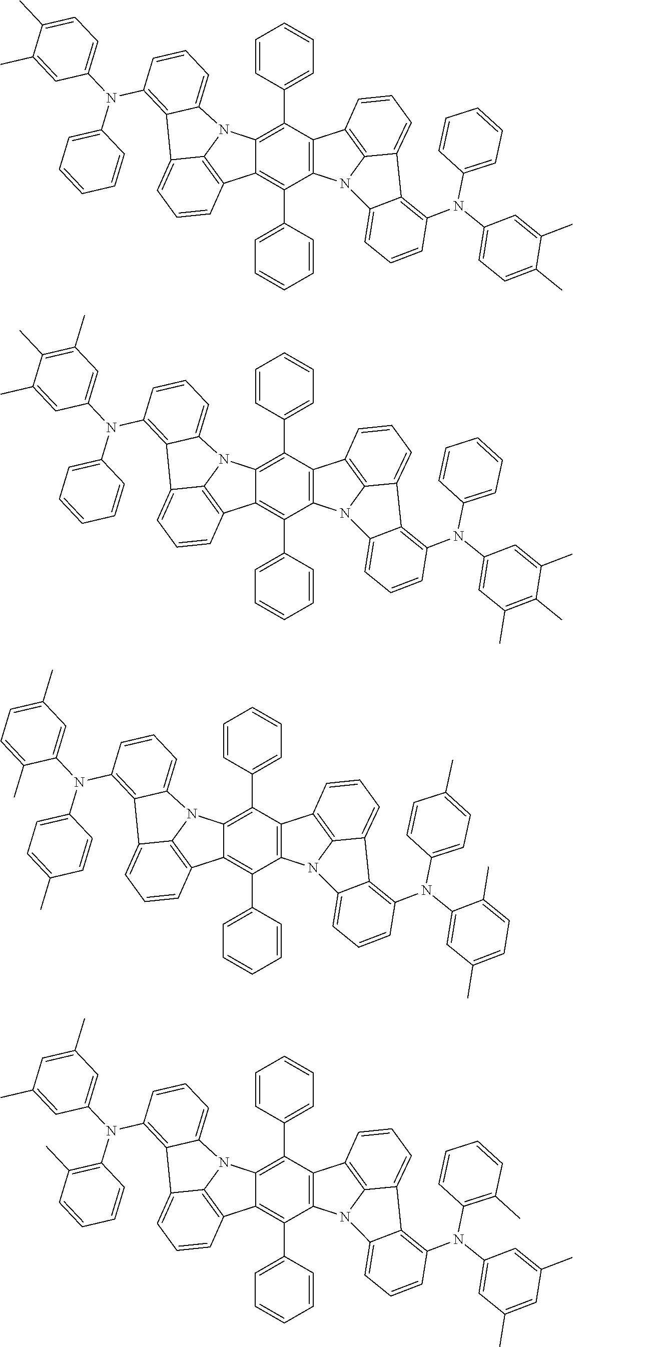

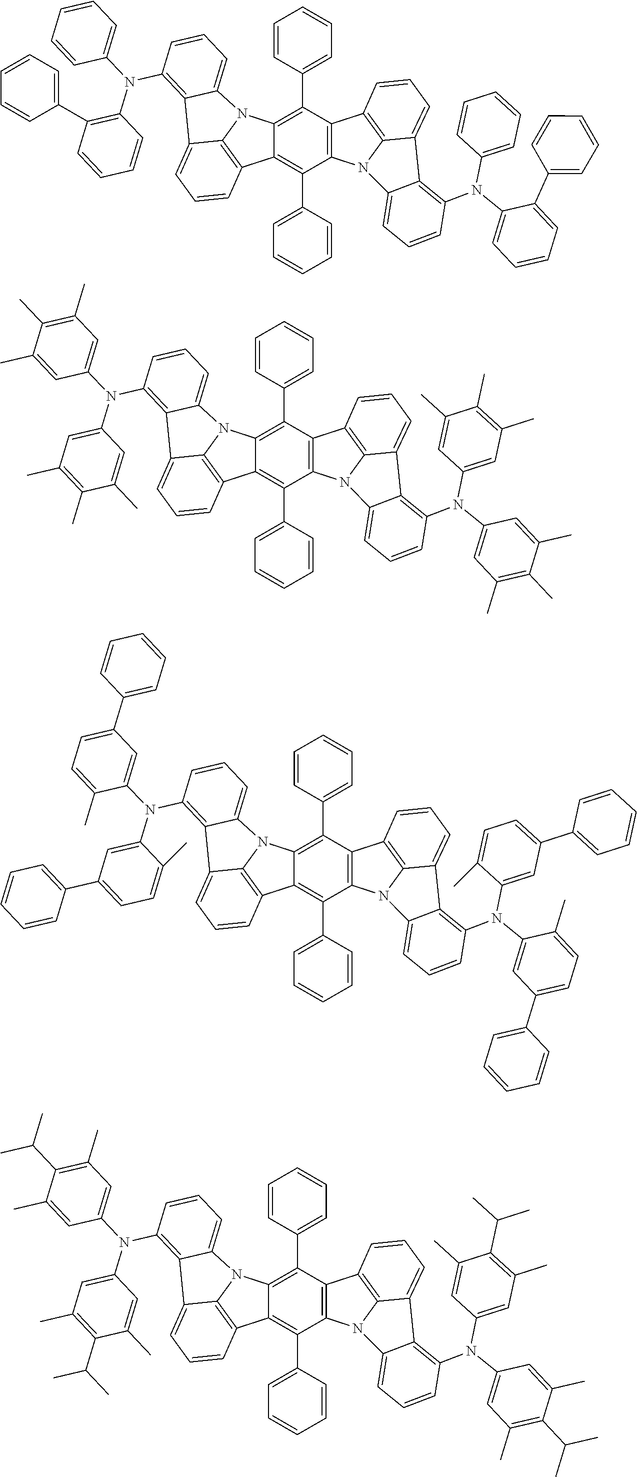

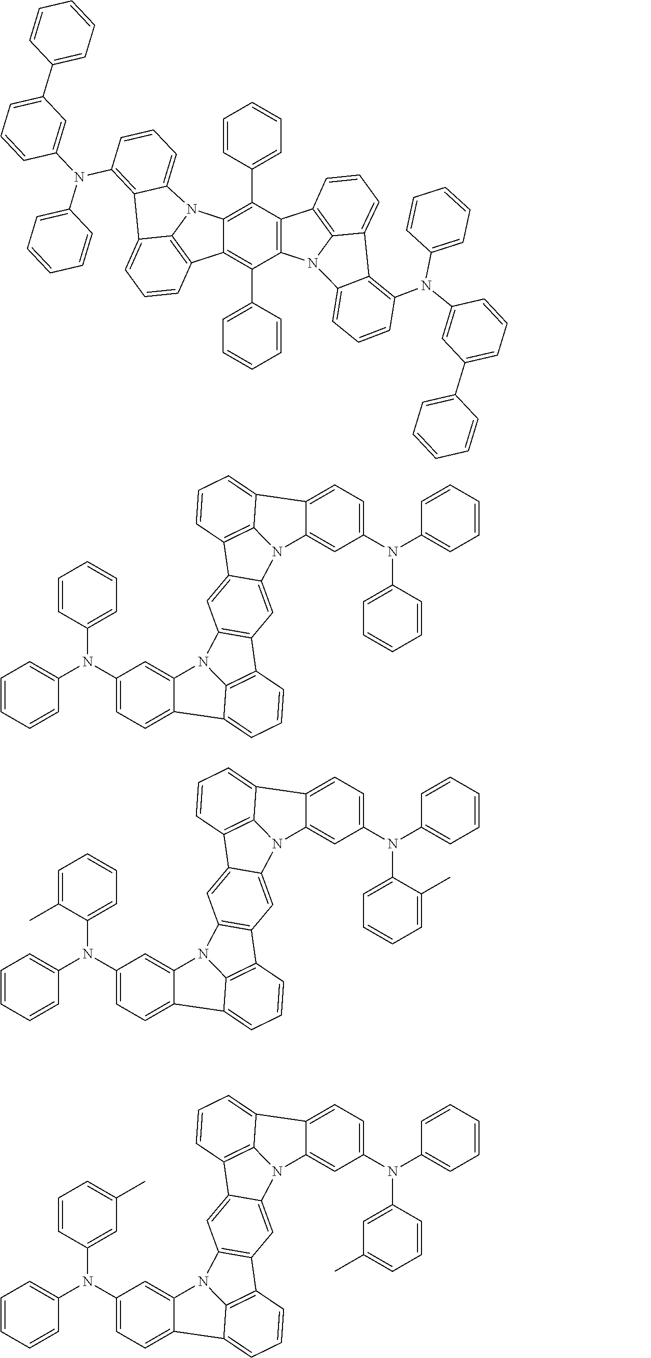

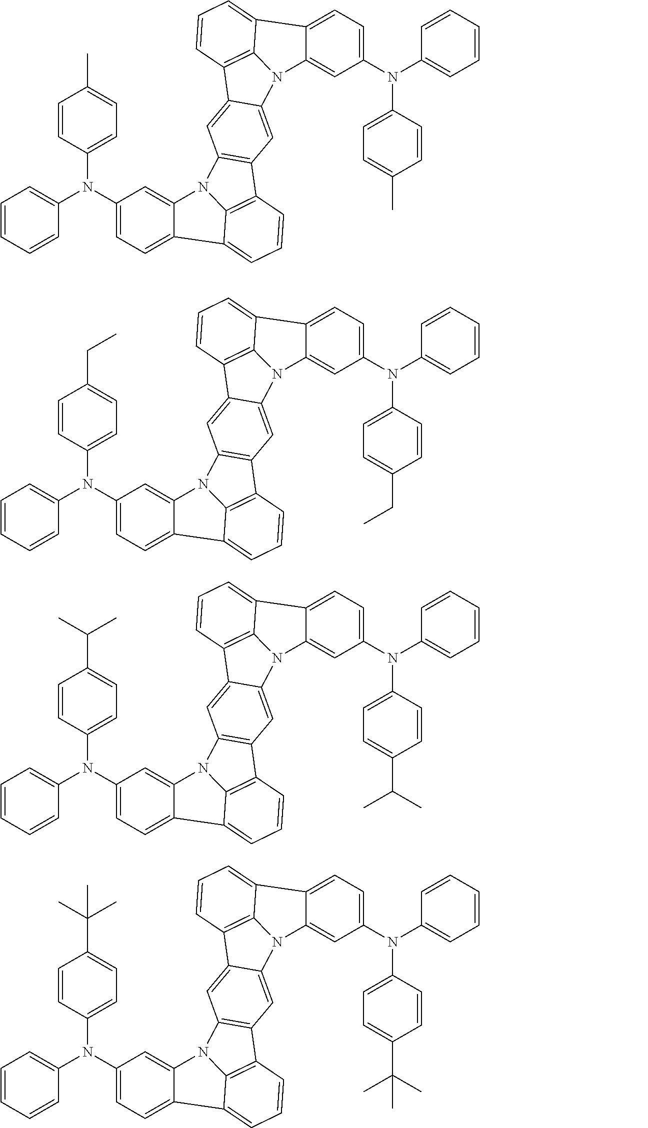

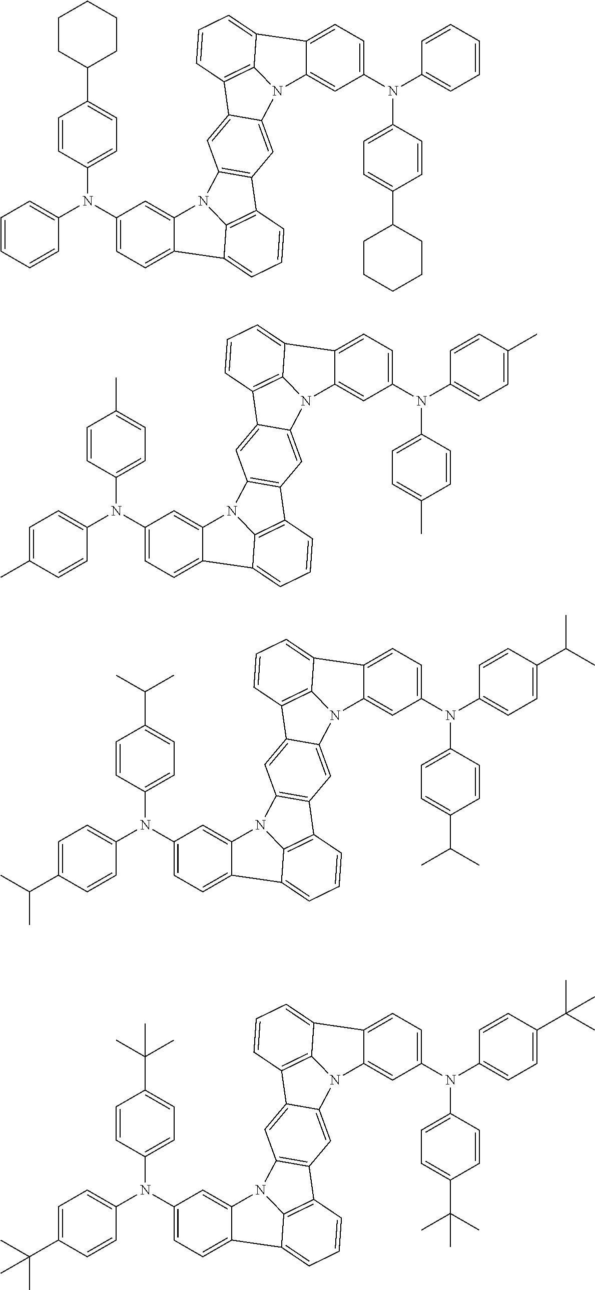

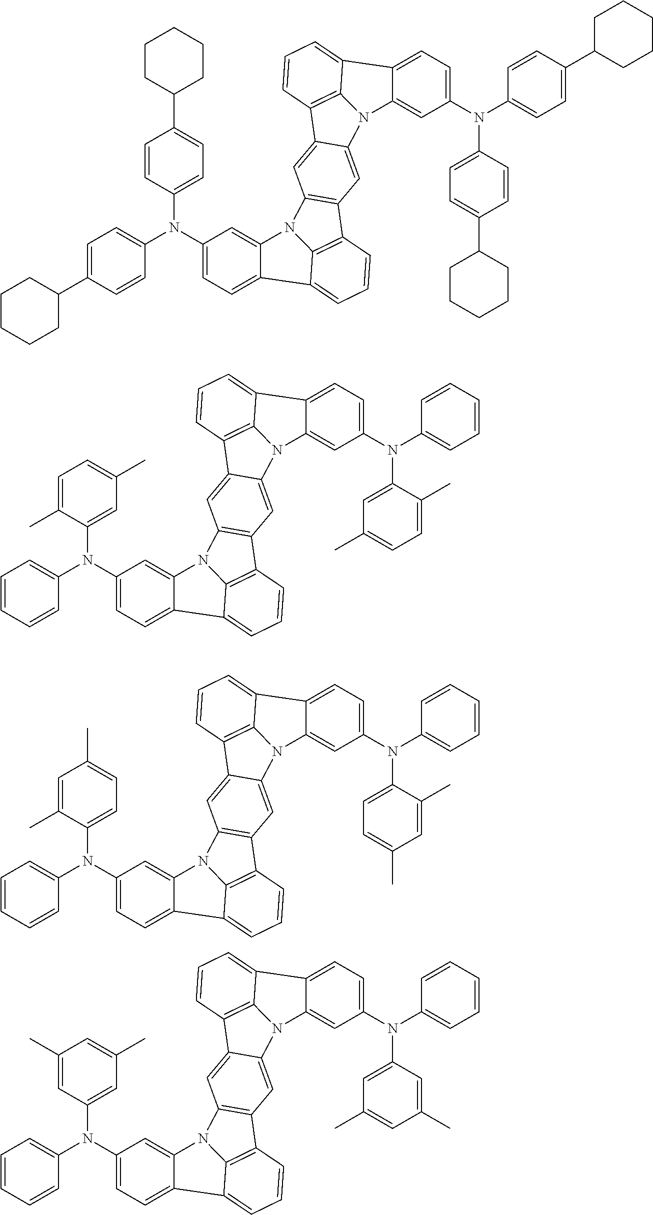

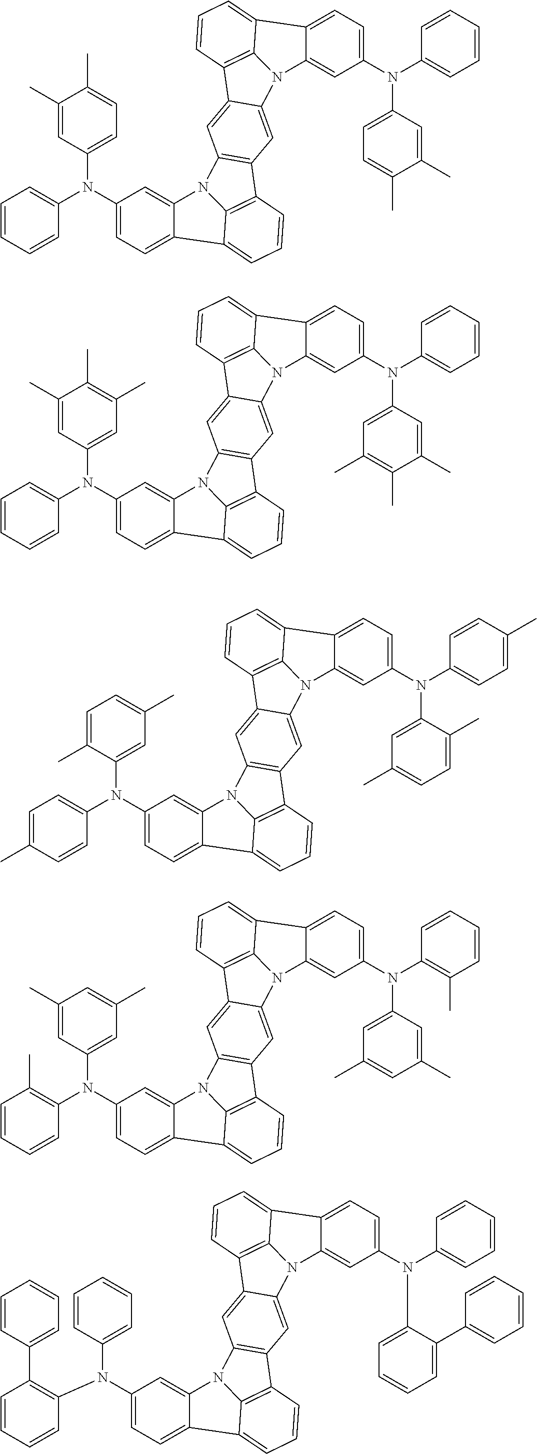

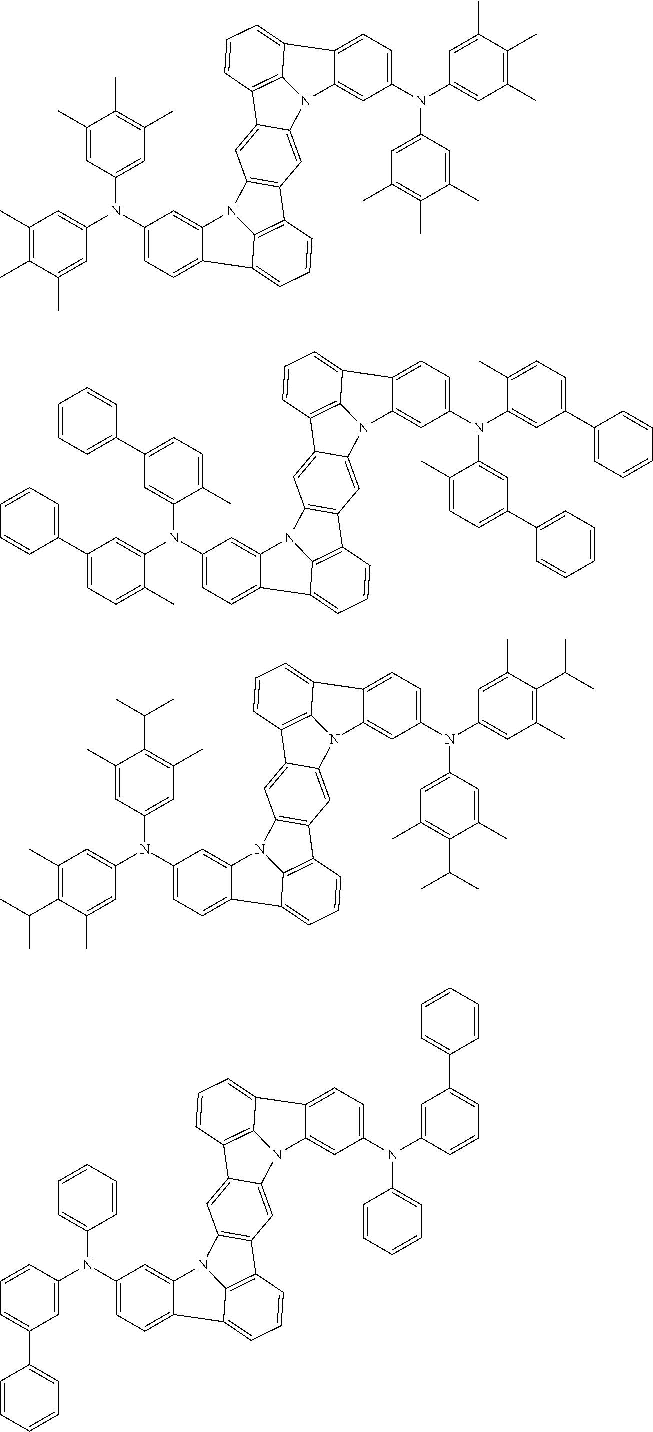

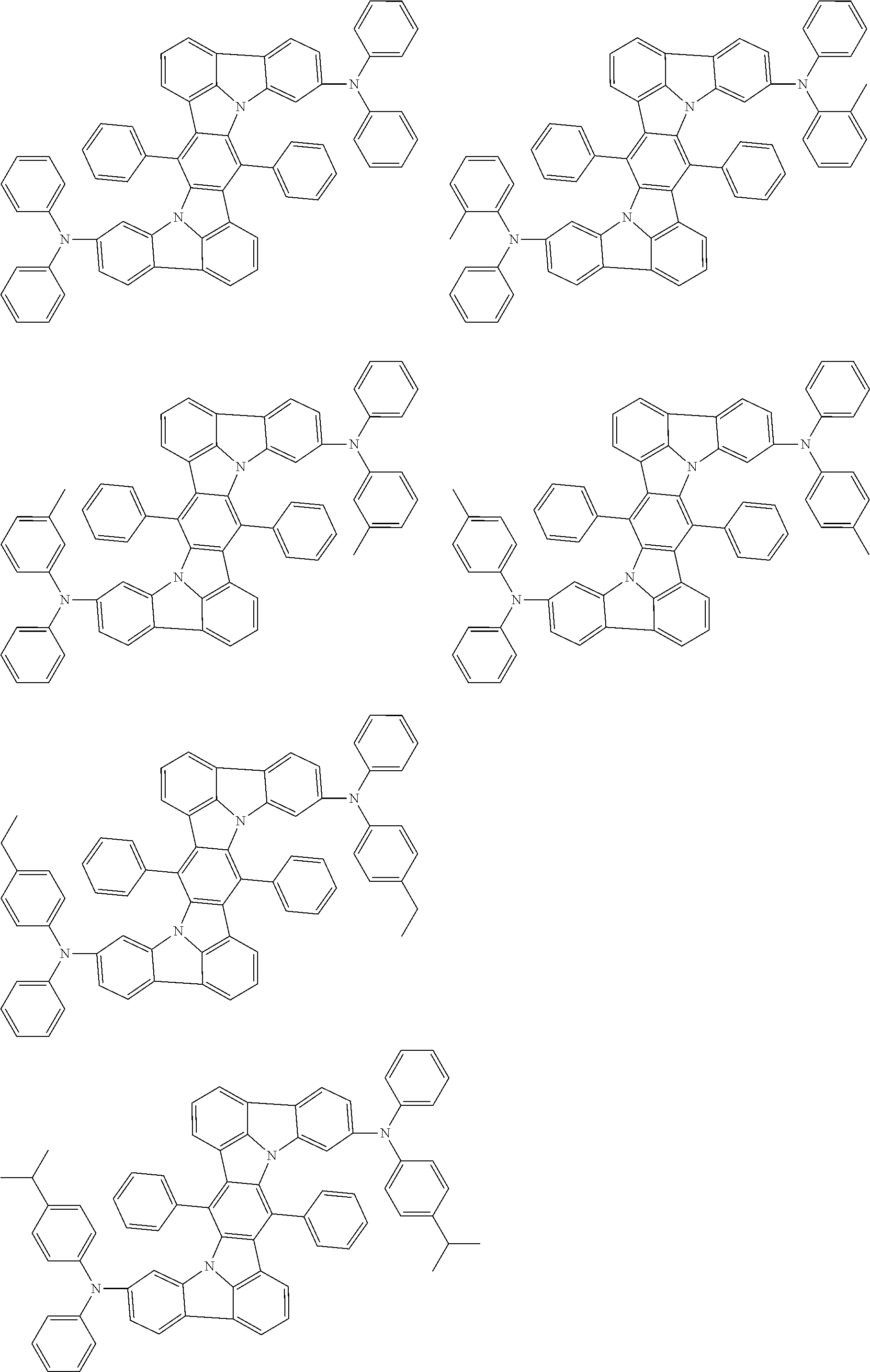

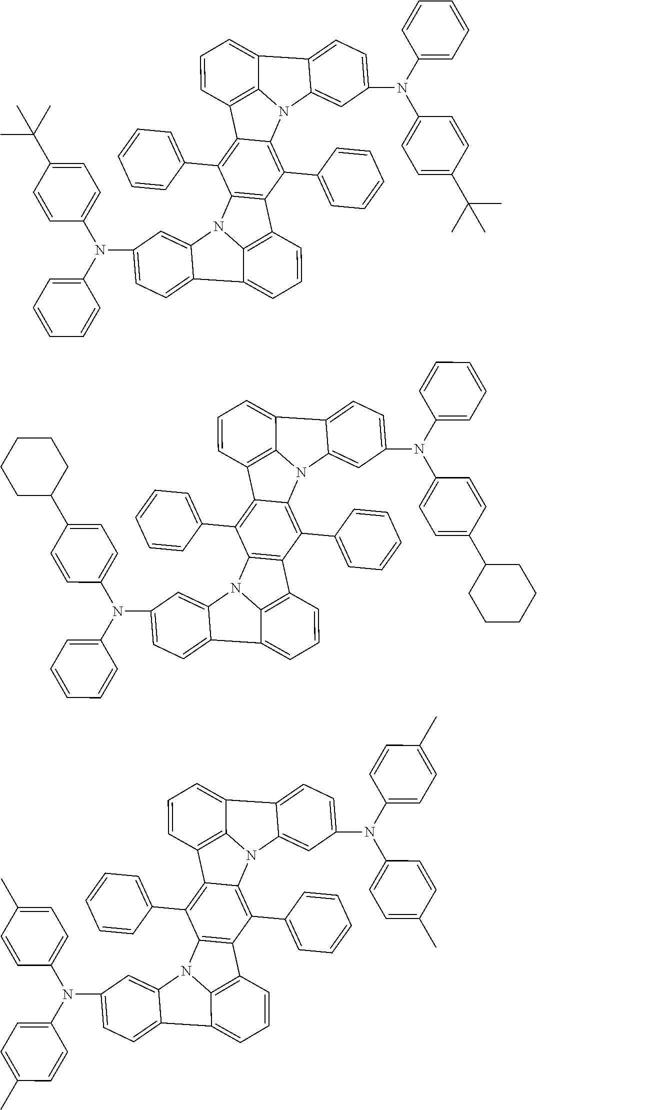

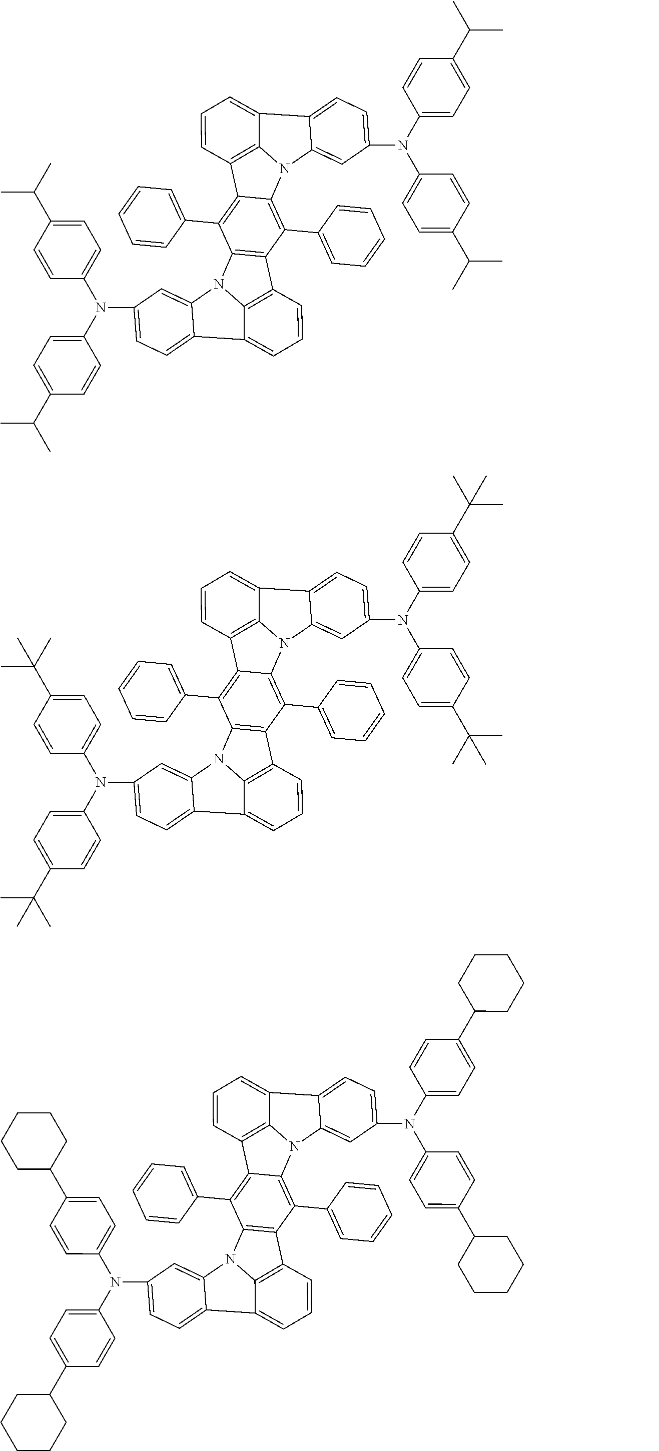

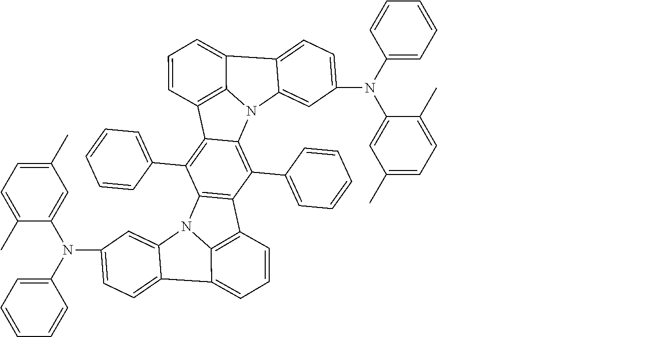 [SMILES: Cc1ccc(C)c(N(c2ccccc2)c2ccc3c4cccc5c6c(-c7ccccc7)c7c(c(-c8ccccc8)c6n(c3c2)c45)c2cccc3c4ccc(N(c5ccccc5)c5cc(C)ccc5C)cc4n7c32)c1]

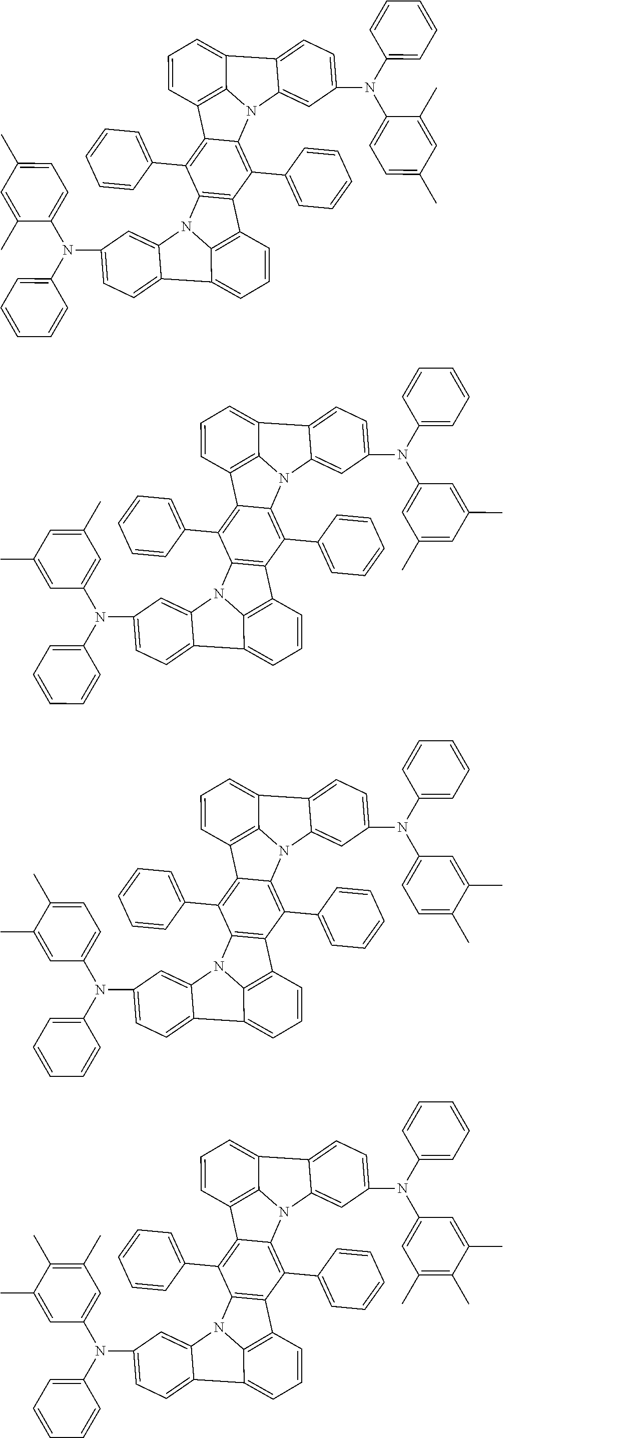

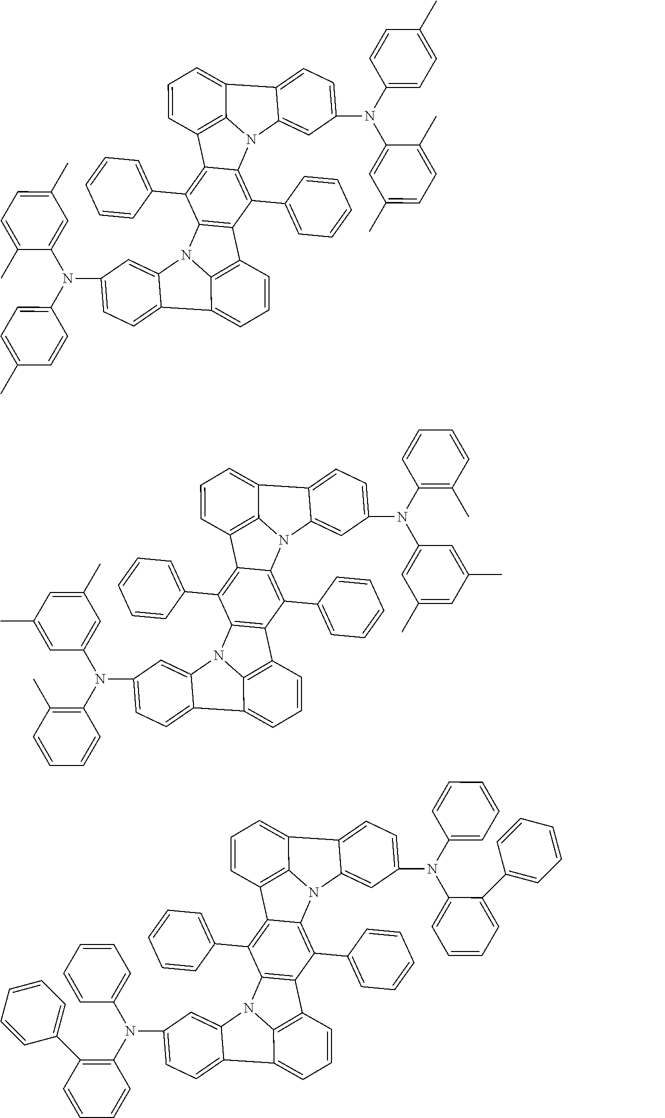

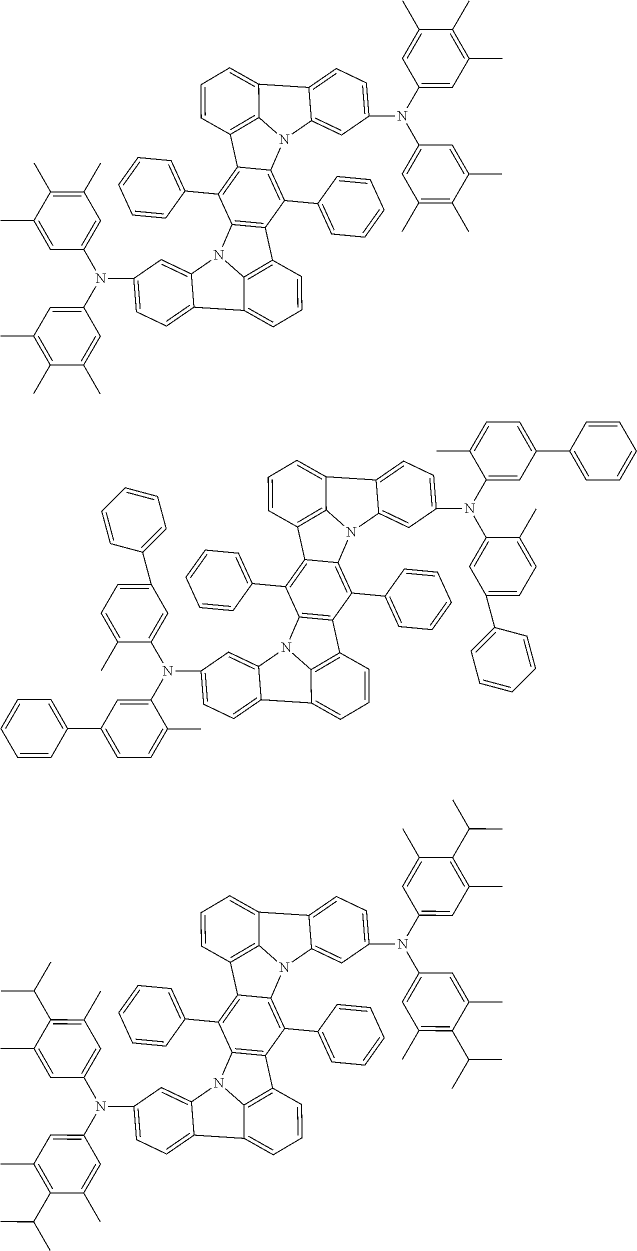

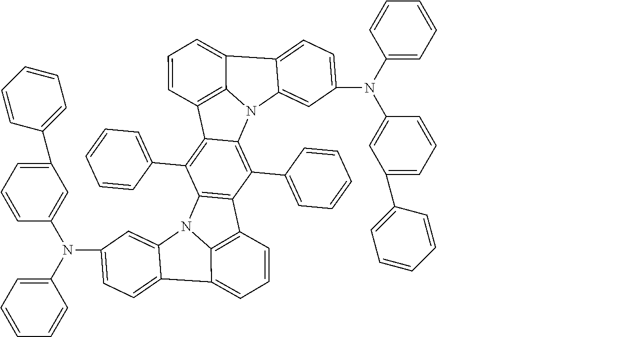 [SMILES: c1ccc(-c2cccc(N(c3ccccc3)c3ccc4c5cccc6c7c(-c8ccccc8)c8c(c(-c9ccccc9)c7n(c4c3)c56)c3cccc4c5ccc(N(c6ccccc6)c6cccc(-c7ccccc7)c6)cc5n8c43)c2)cc1]

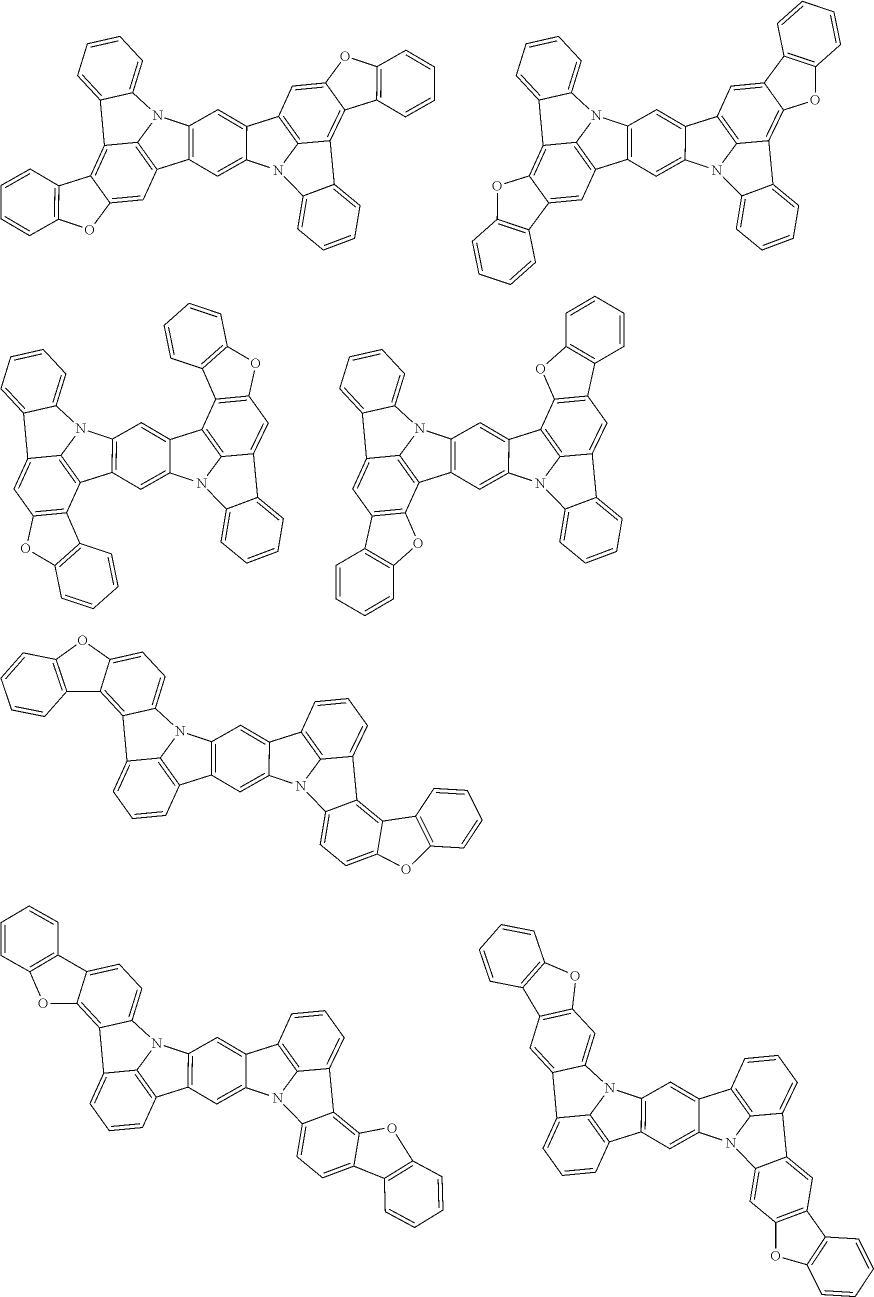

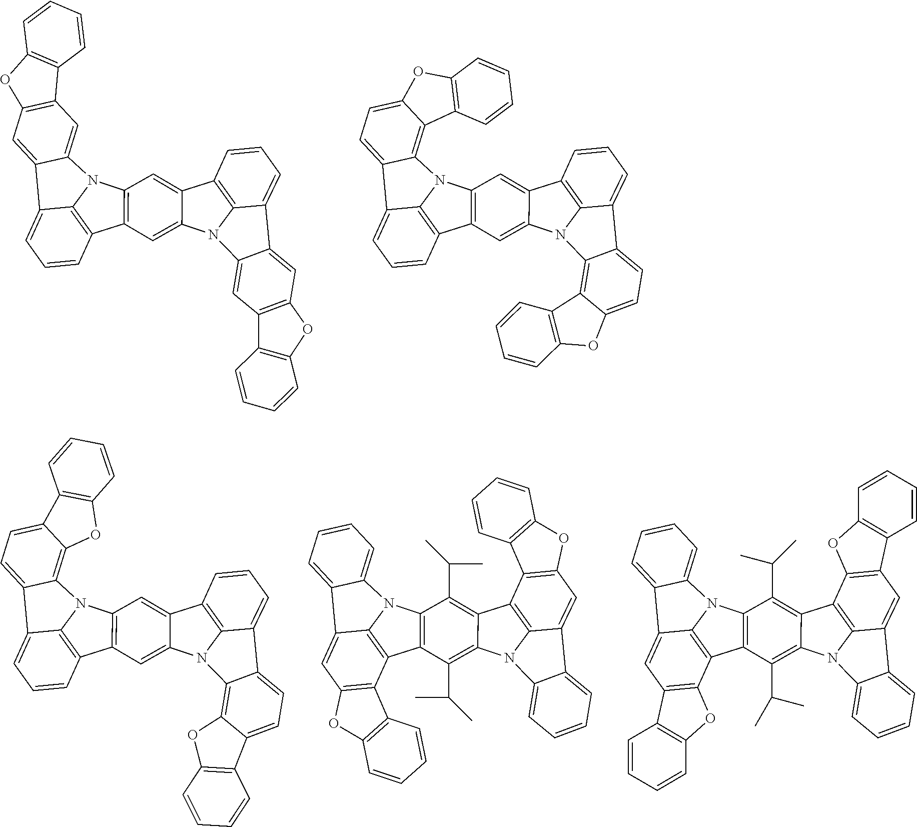

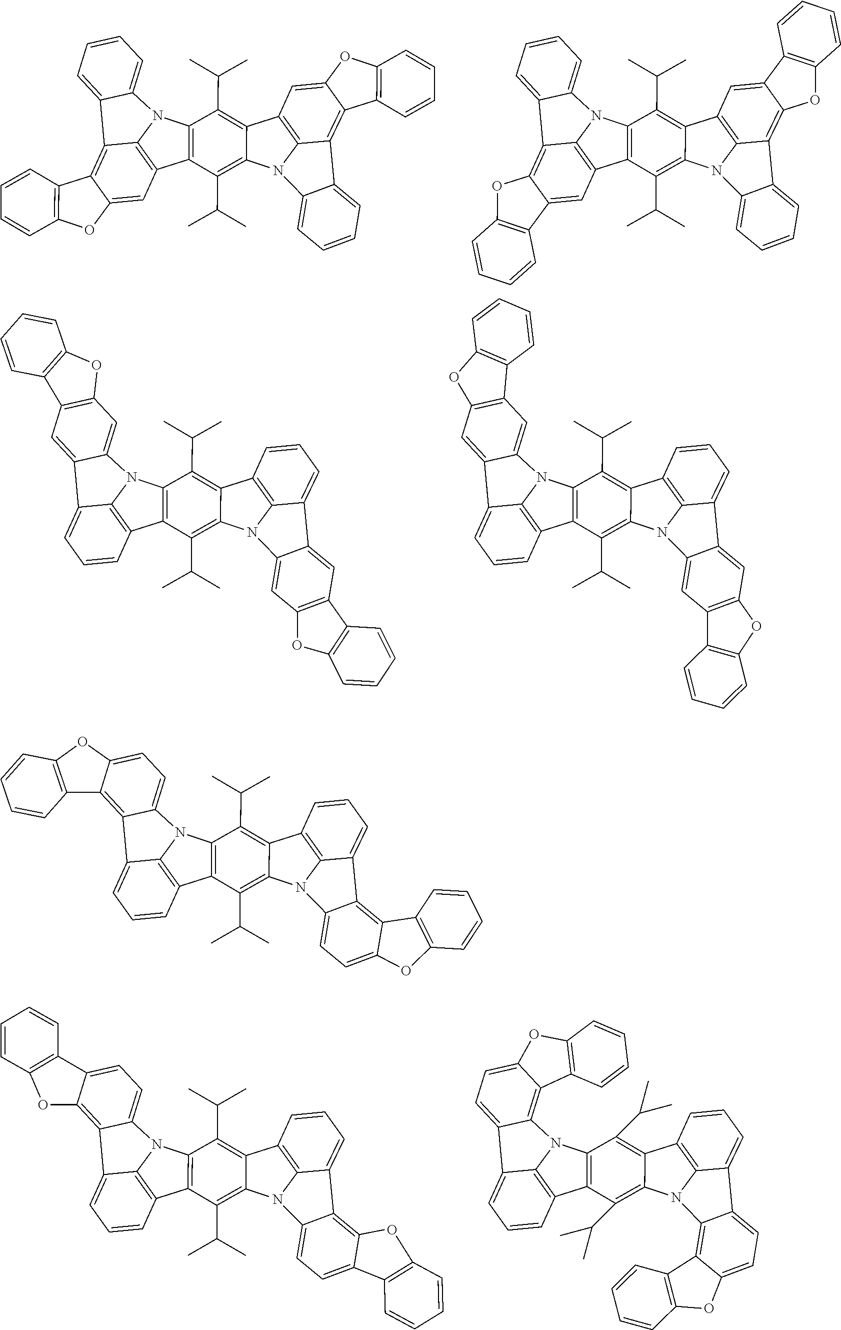

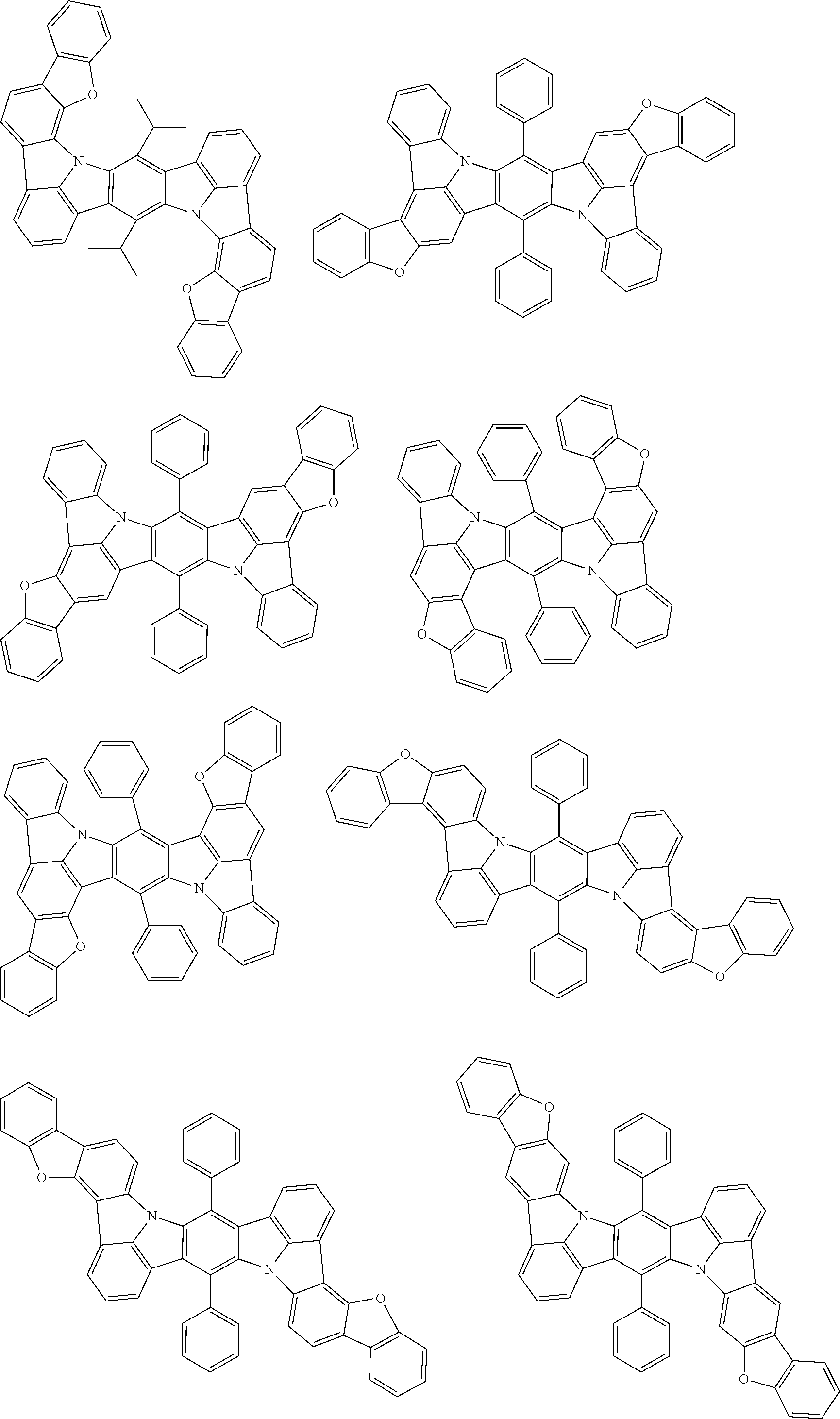

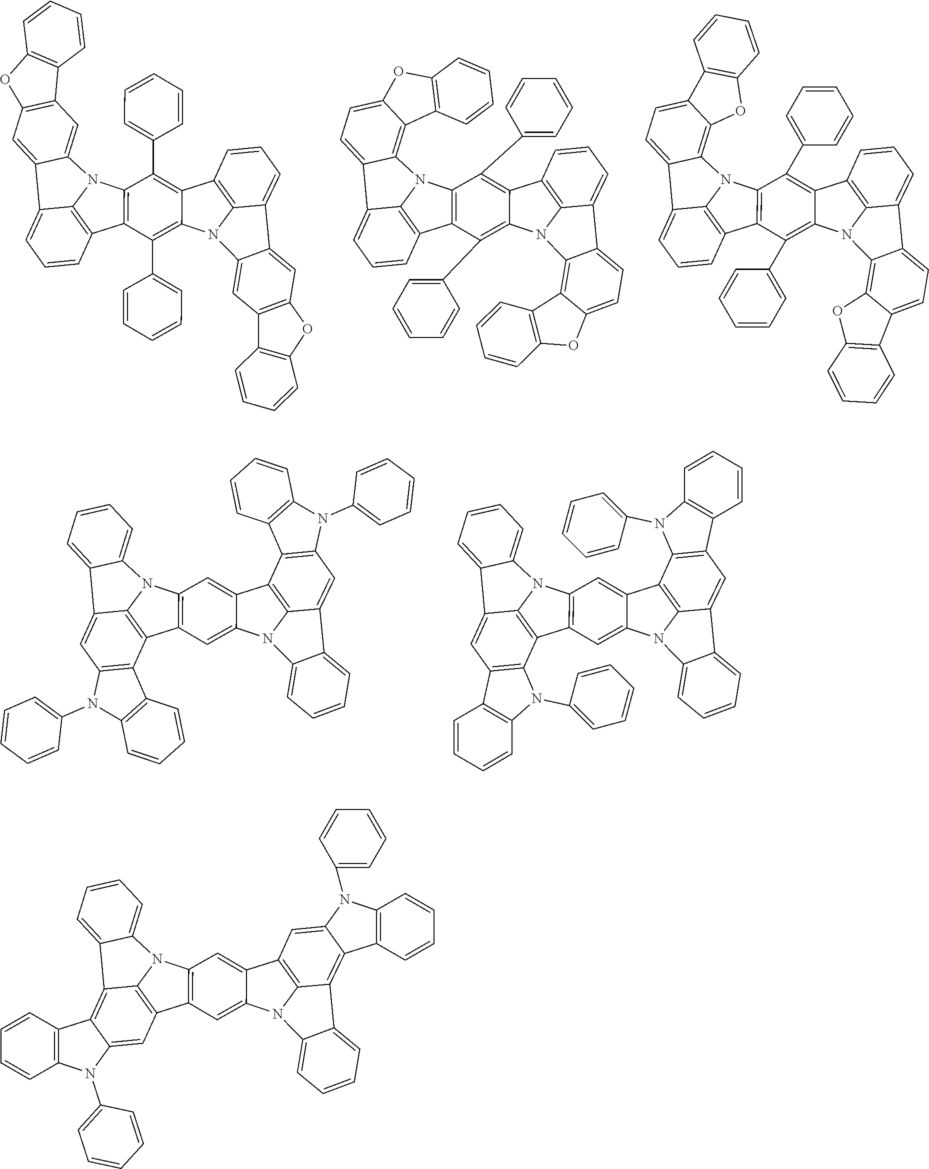

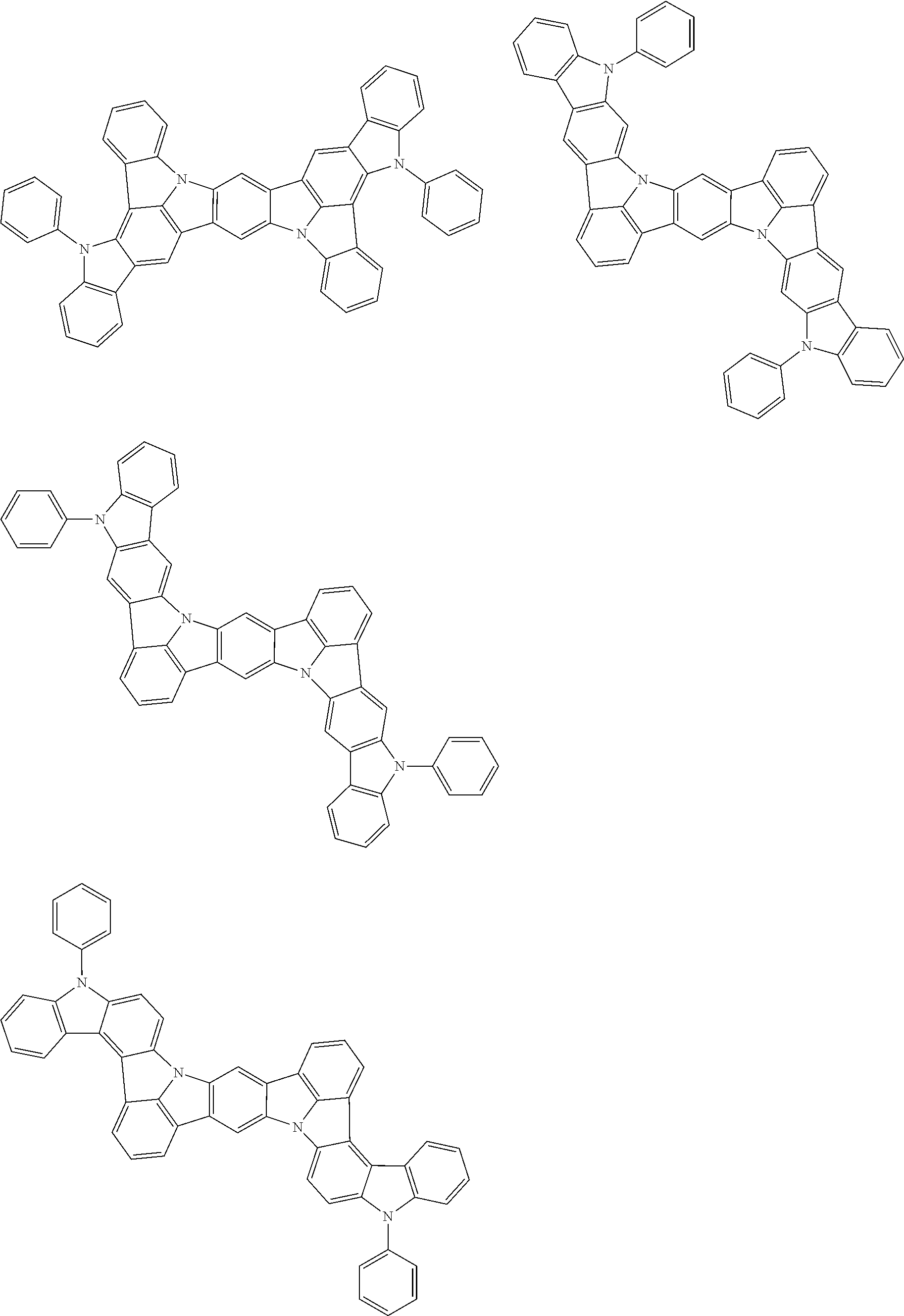 [SMILES: c1ccc(-n2c3ccccc3c3cc4c5cc6c(cc5n5c7ccccc7c(c32)c45)c2cc3c4ccccc4n(-c4ccccc4)c3c3c4ccccc4n6c23)cc1 ; c1ccc(-n2c3ccccc3c3cc4c5cccc6c7cc8c(cc7n(c4cc32)c65)c2cccc3c4cc5c6ccccc6n(-c6ccccc6)c5cc4n8c23)cc1 ; c1ccc(-n2c3ccccc3c3cc4c(cc32)c2cccc3c5cc6c(cc5n4c23)c2cccc3c4cc5c(cc4n6c32)c2ccccc2n5-c2ccccc2)cc1 ; c1ccc(-n2c3ccccc3c3c4c5cccc6c7cc8c(cc7n(c4ccc32)c65)c2cccc3c4c5c6ccccc6n(-c6ccccc6)c5ccc4n8c23)cc1]

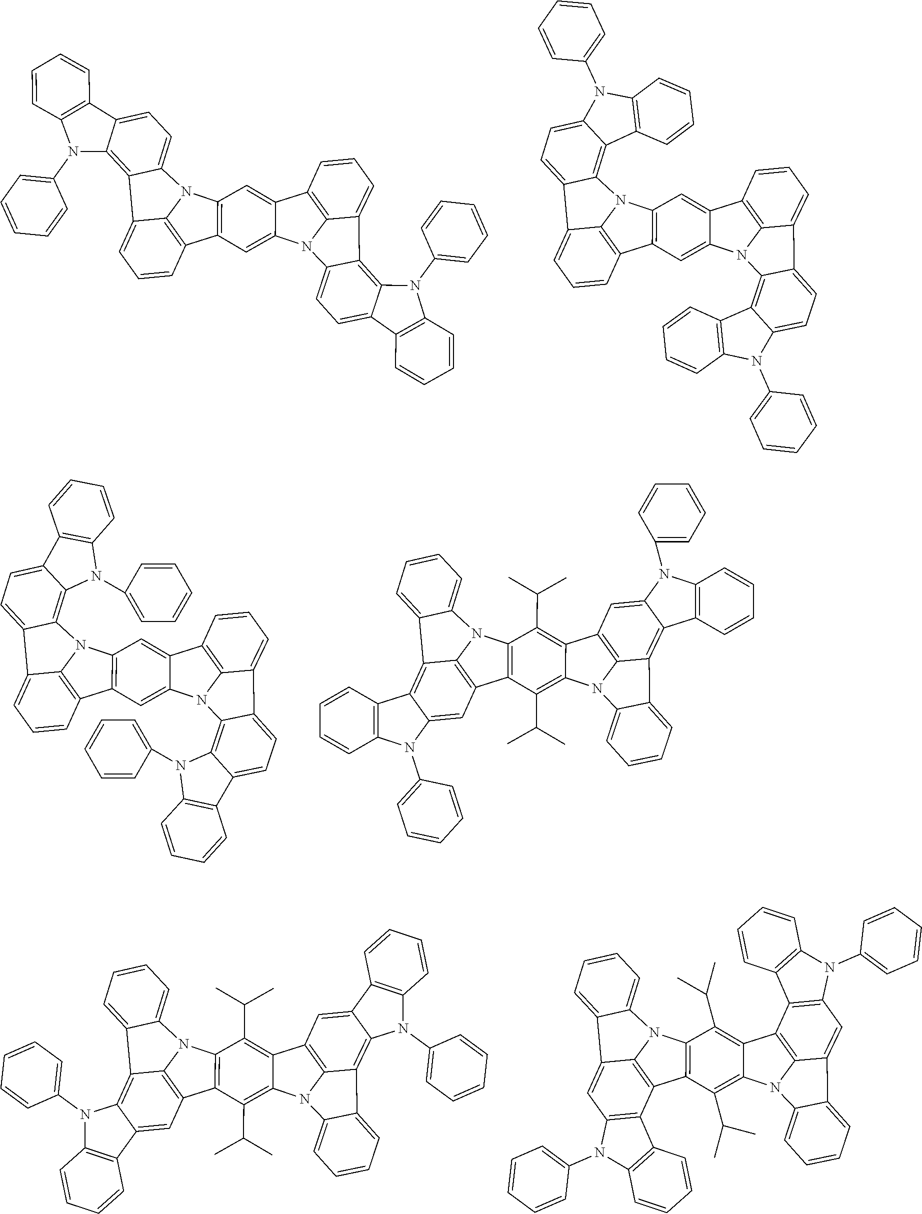

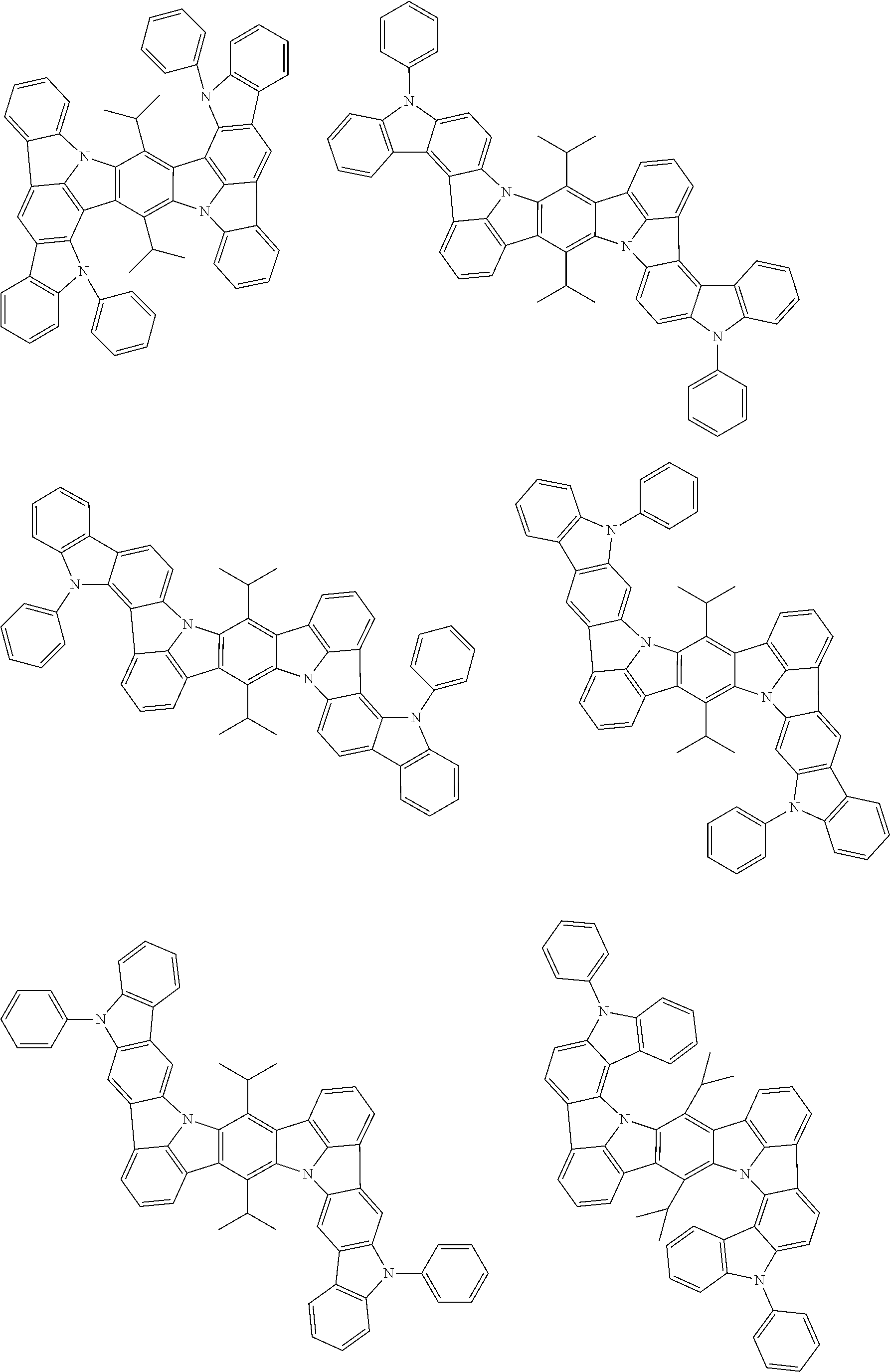

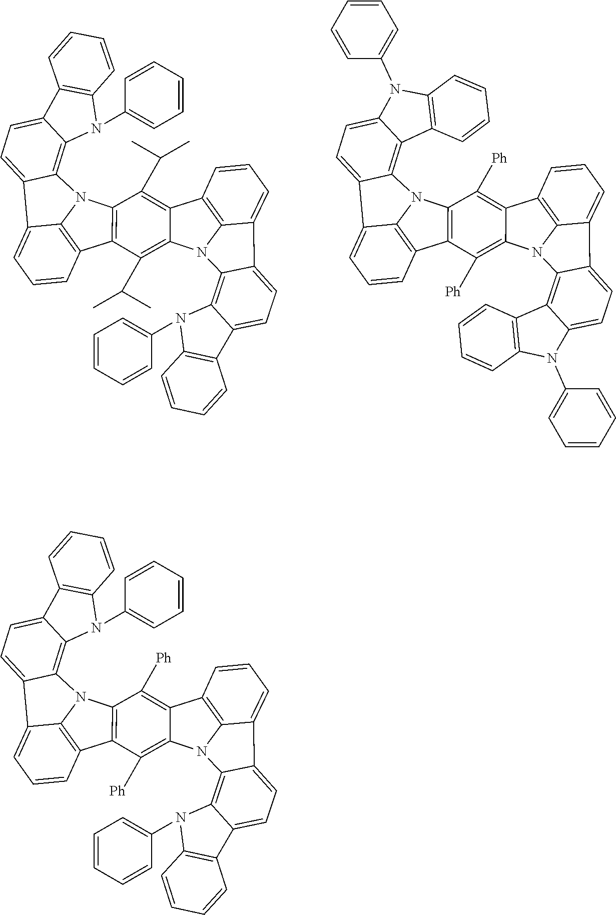 [SMILES: CC(C)c1c2c3cccc4c5ccc6c7ccccc7n(-c7ccccc7)c6c5n(c2c(C(C)C)c2c5cccc6c7ccc8c9ccccc9n(-c9ccccc9)c8c7n(c12)c65)c43 ; c1ccc(-c2c3c4cccc5c6ccc7c(c8ccccc8n7-c7ccccc7)c6n(c3c(-c3ccccc3)c3c6cccc7c8ccc9c(c%10ccccc%10n9-c9ccccc9)c8n(c23)c76)c54)cc1 ; c1ccc(-c2c3c4cccc5c6ccc7c8ccccc8n(-c8ccccc8)c7c6n(c3c(-c3ccccc3)c3c6cccc7c8ccc9c%10ccccc%10n(-c%10ccccc%10)c9c8n(c23)c76)c54)cc1]

(In the formulas, “Ph” represents a phenyl group)

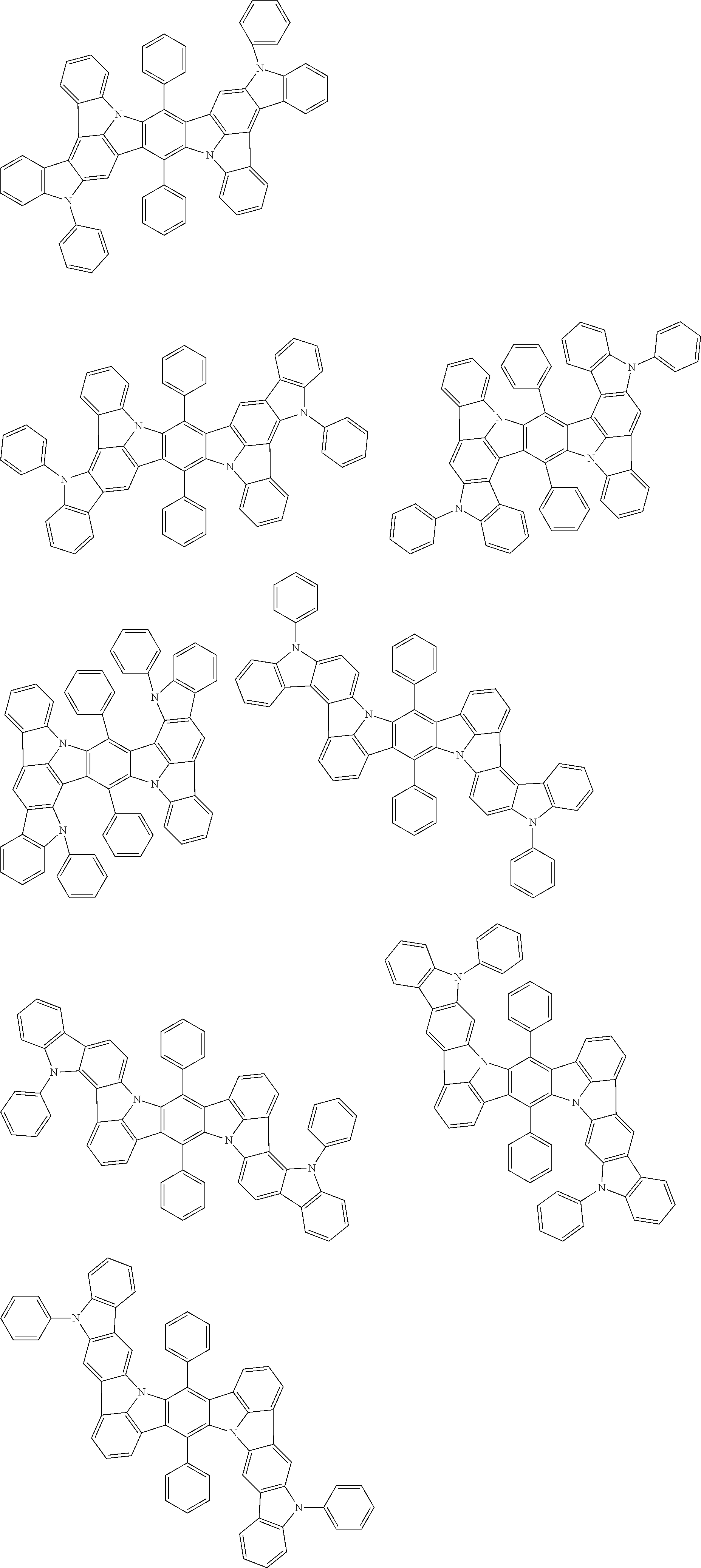

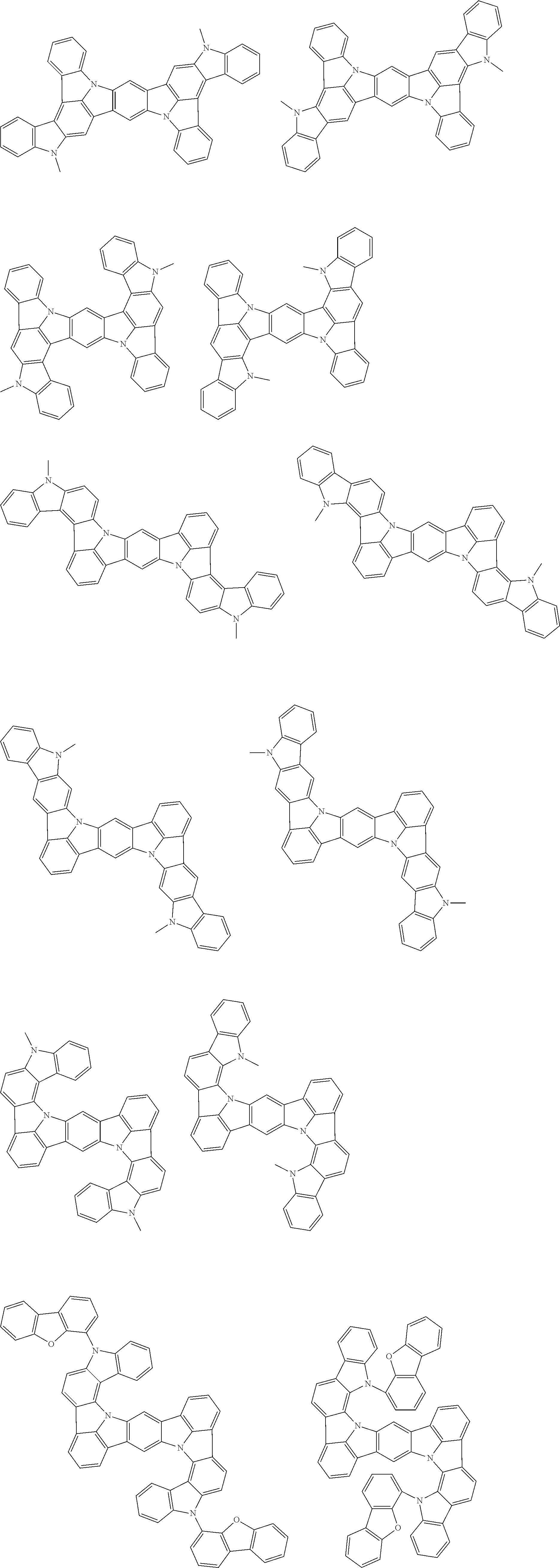

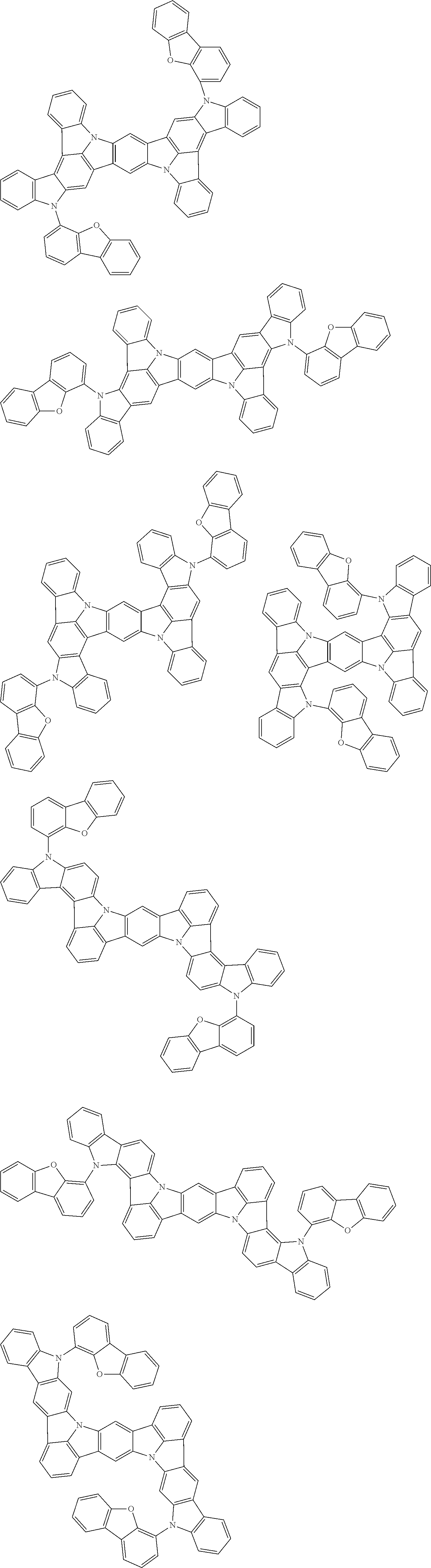

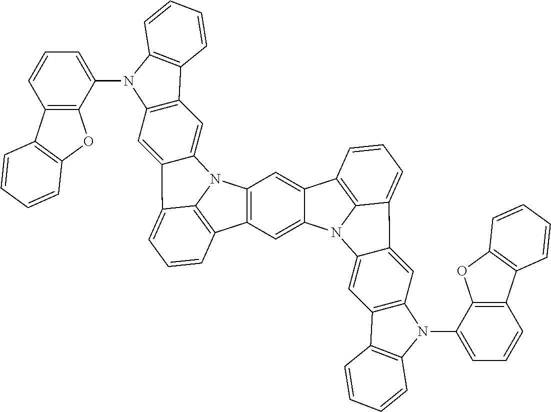 [SMILES: c1ccc2c(c1)oc1c(-n3c4ccccc4c4cc5c(cc43)c3cccc4c6cc7c(cc6n5c34)c3cccc4c5cc6c(cc5n7c43)c3ccccc3n6-c3cccc4c3oc3ccccc34)cccc12]

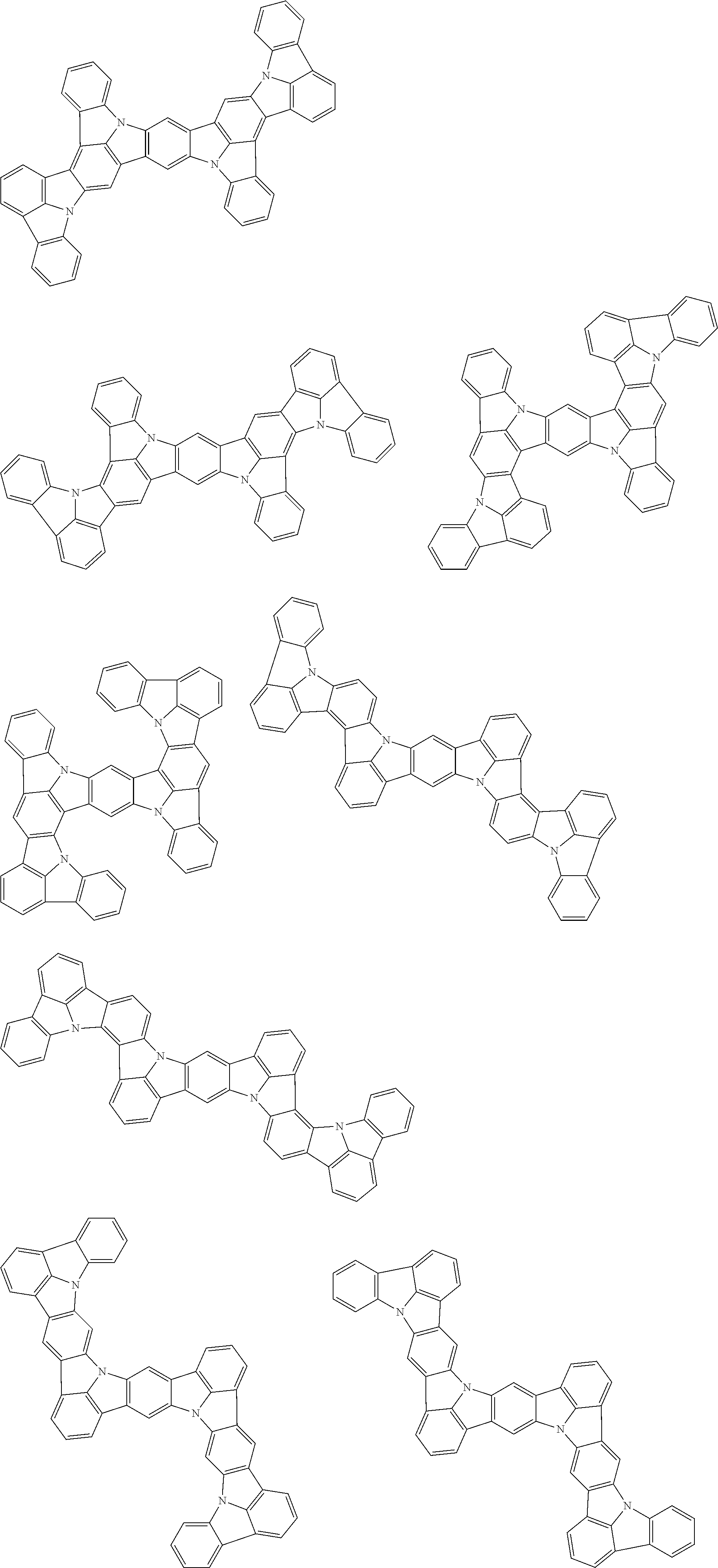

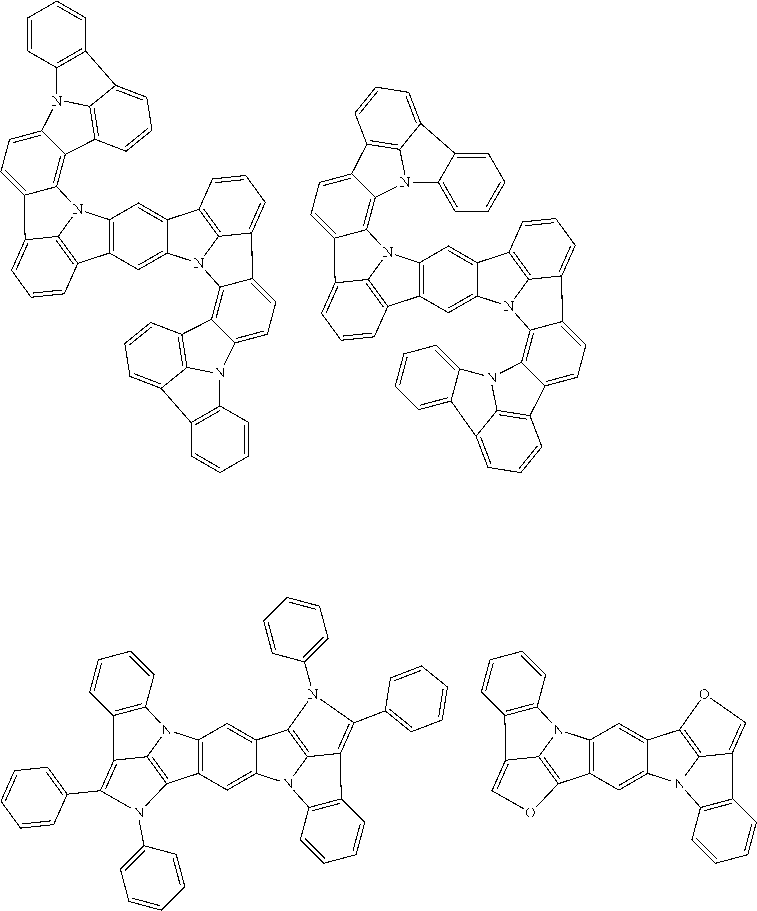

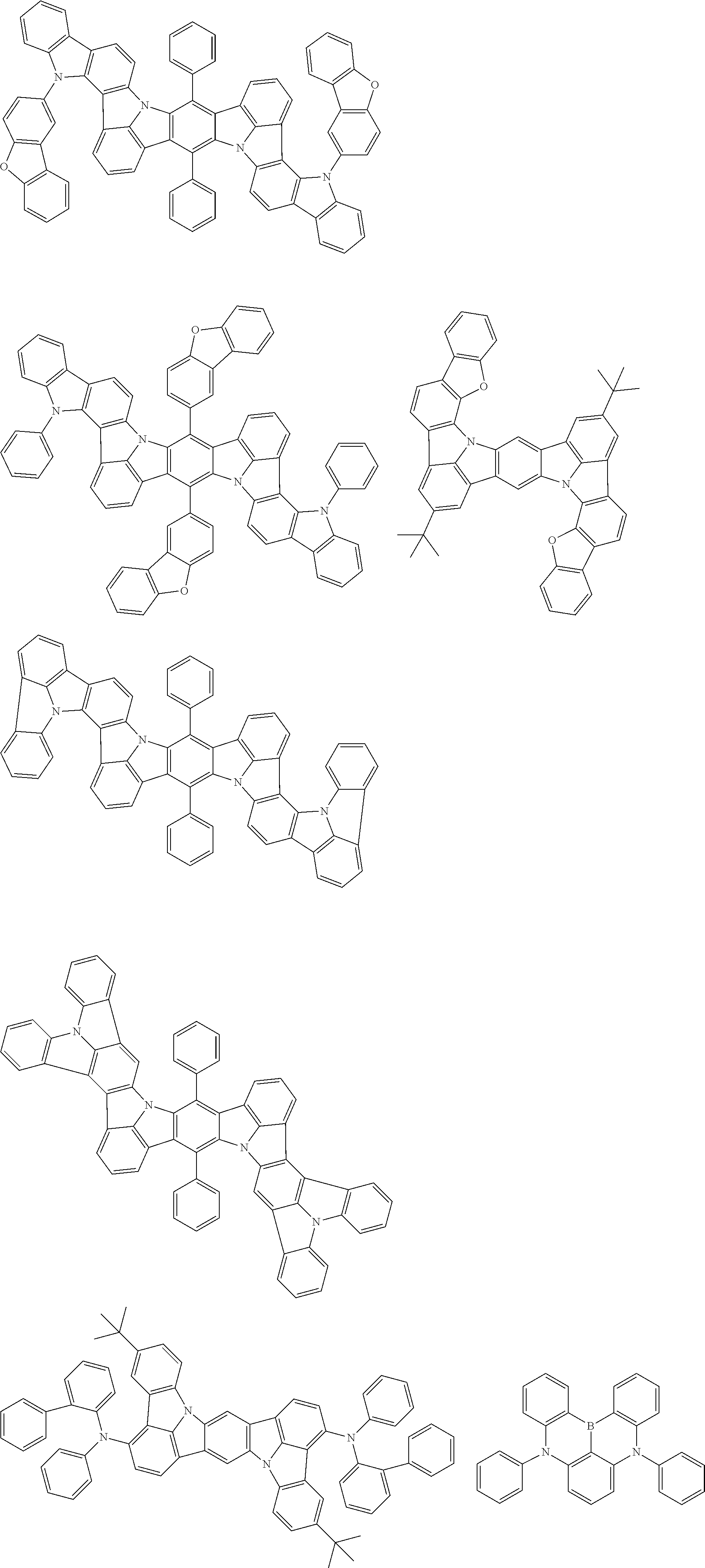

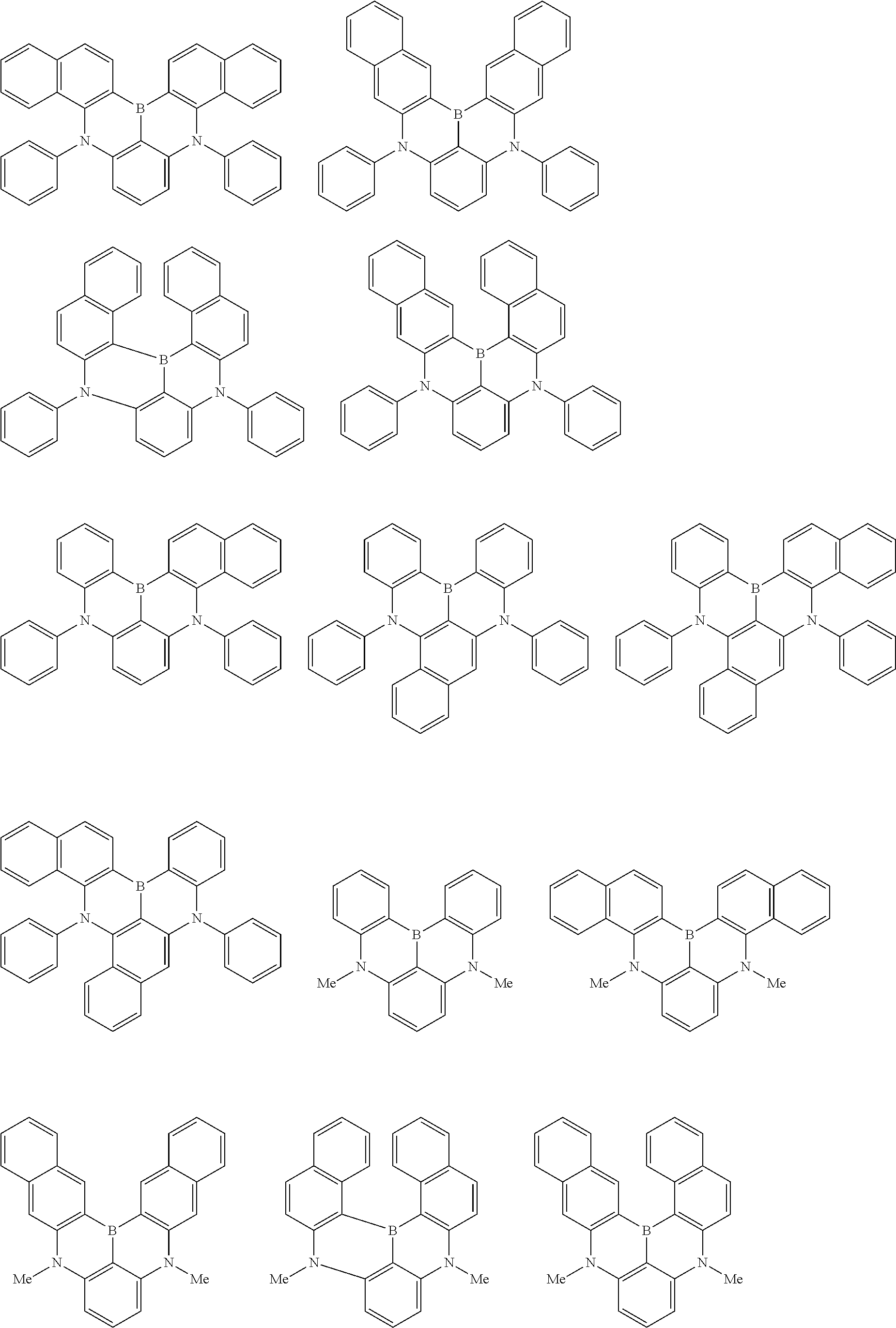

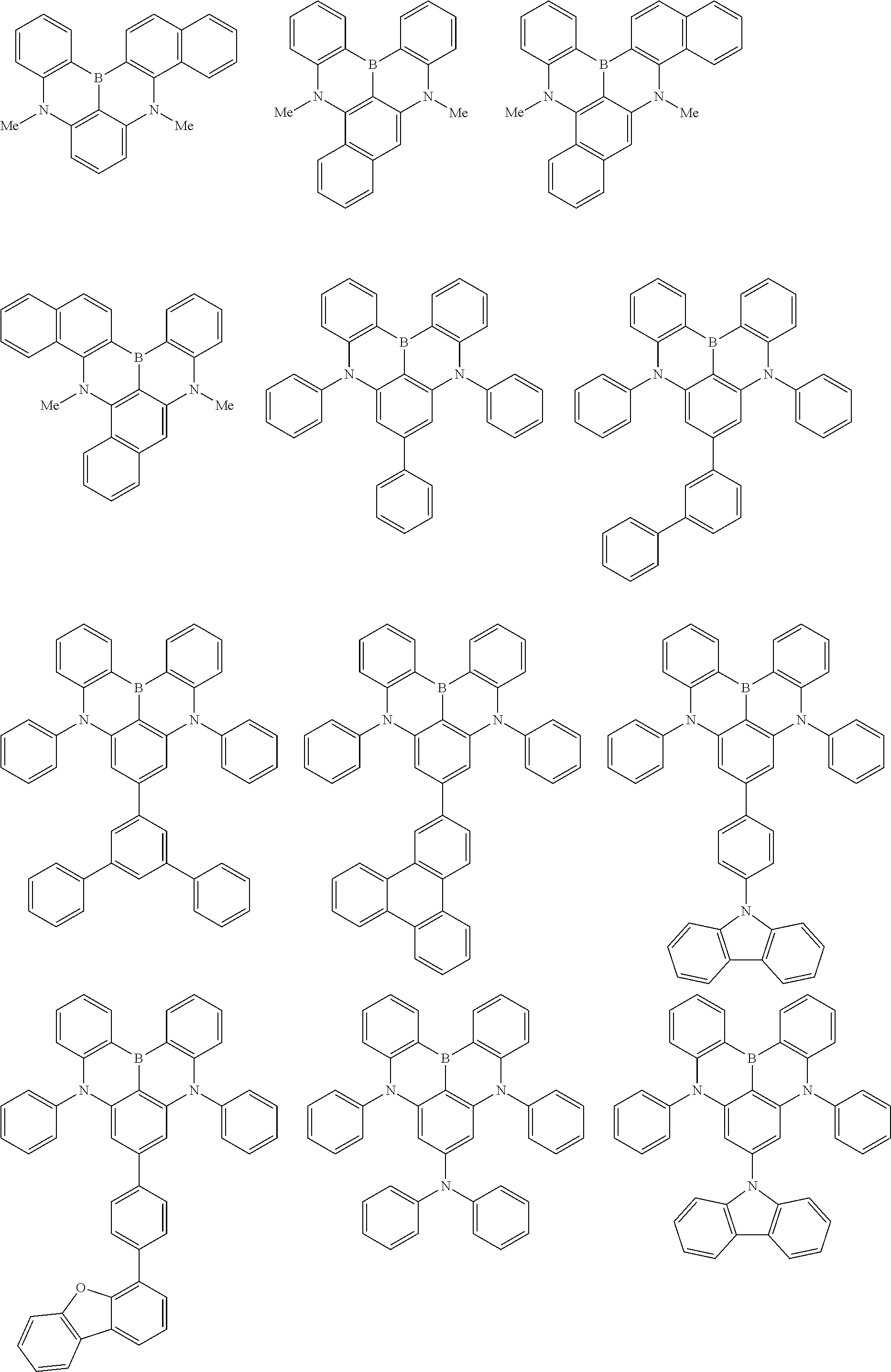

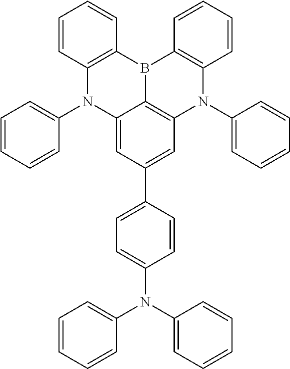 [SMILES: c1ccc(N(c2ccccc2)c2ccc(-c3cc4c5c(c3)N(c3ccccc3)c3ccccc3B5c3ccccc3N4c3ccccc3)cc2)cc1]

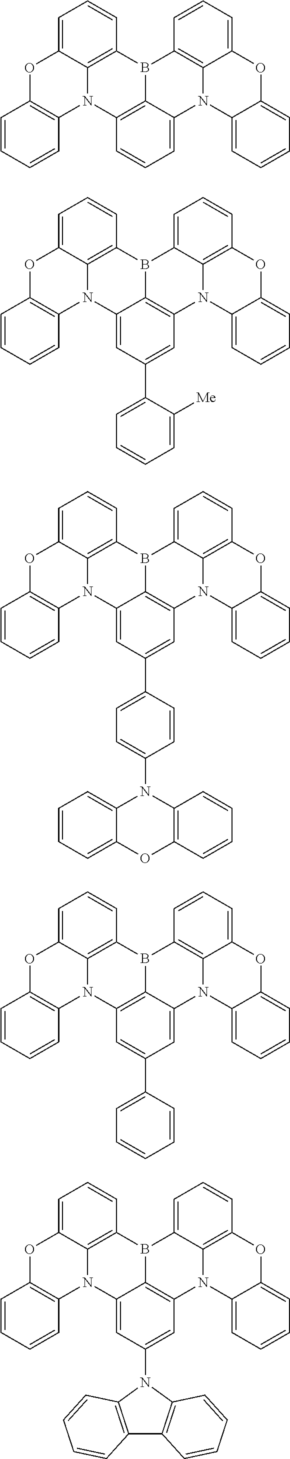

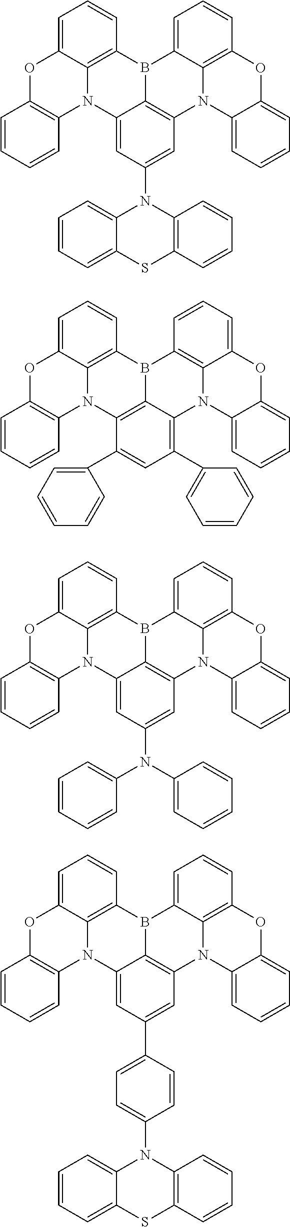

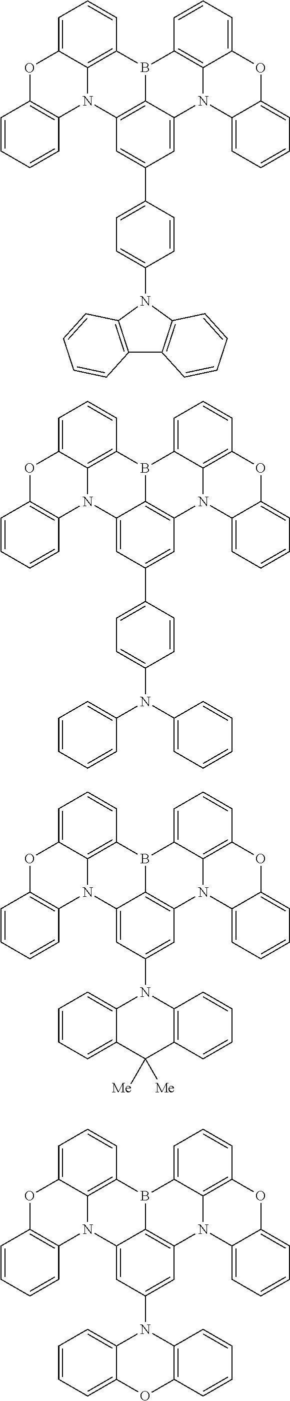

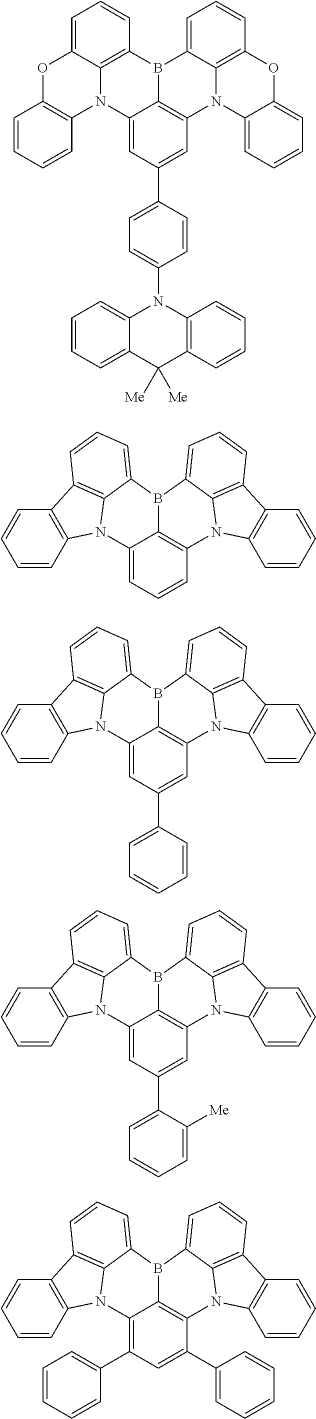

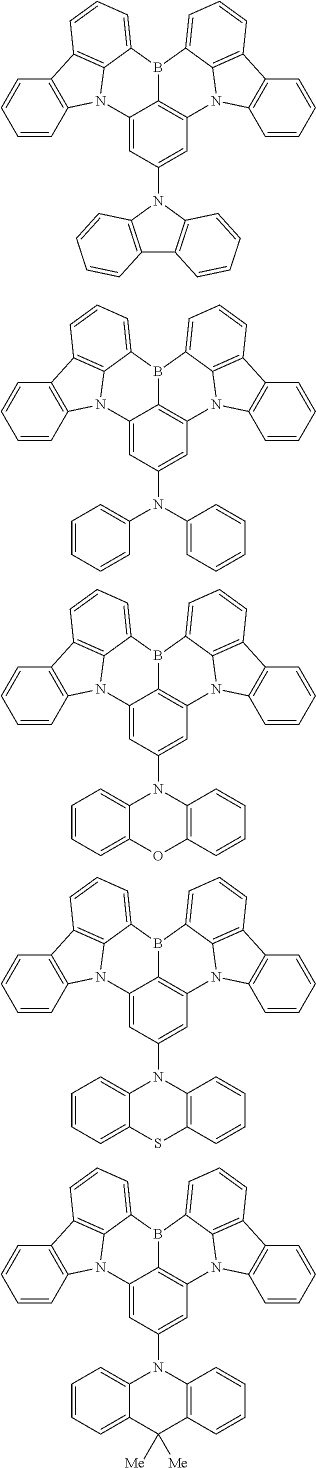

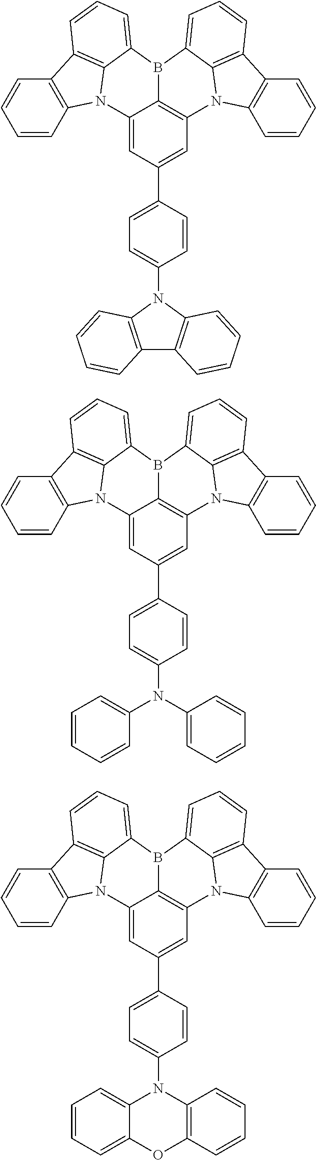

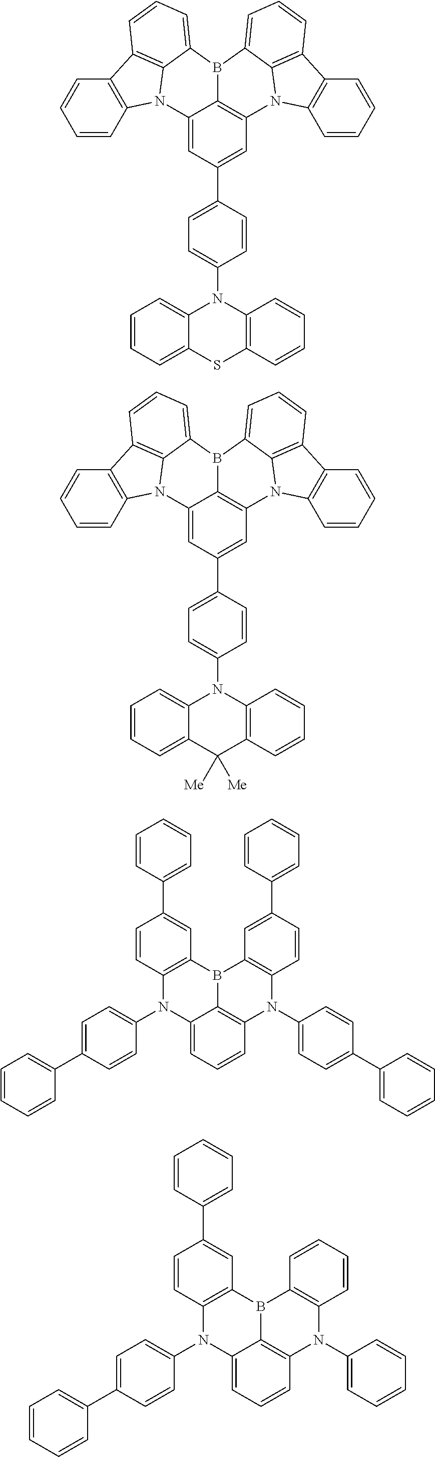

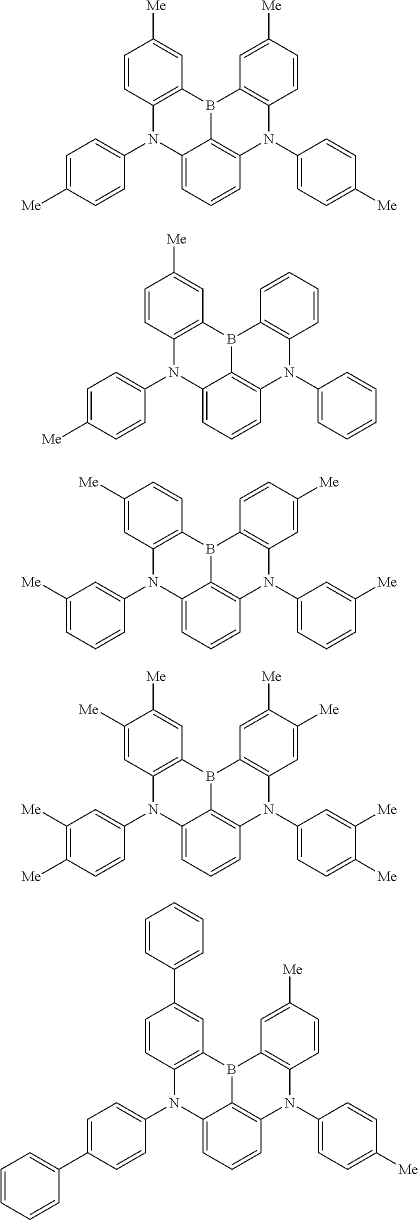

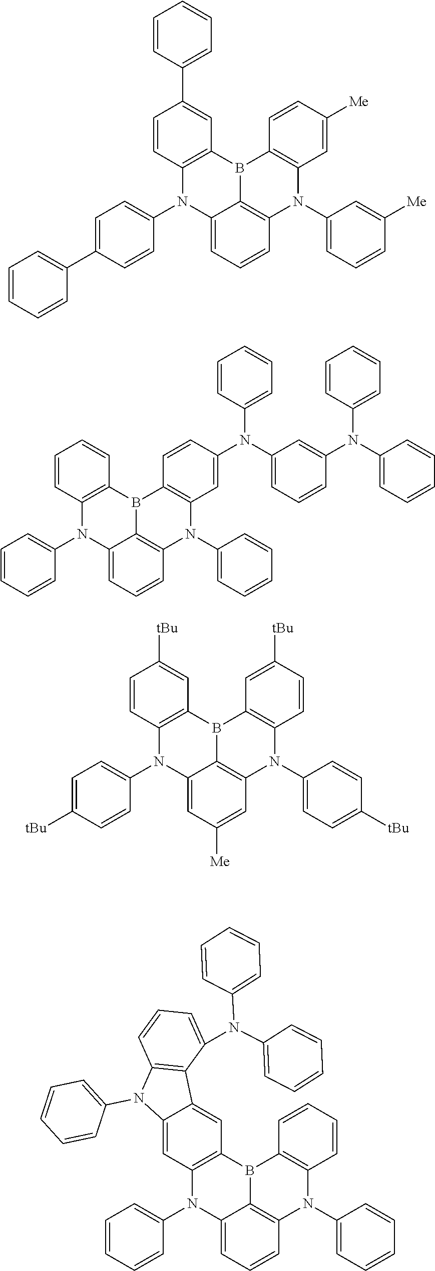

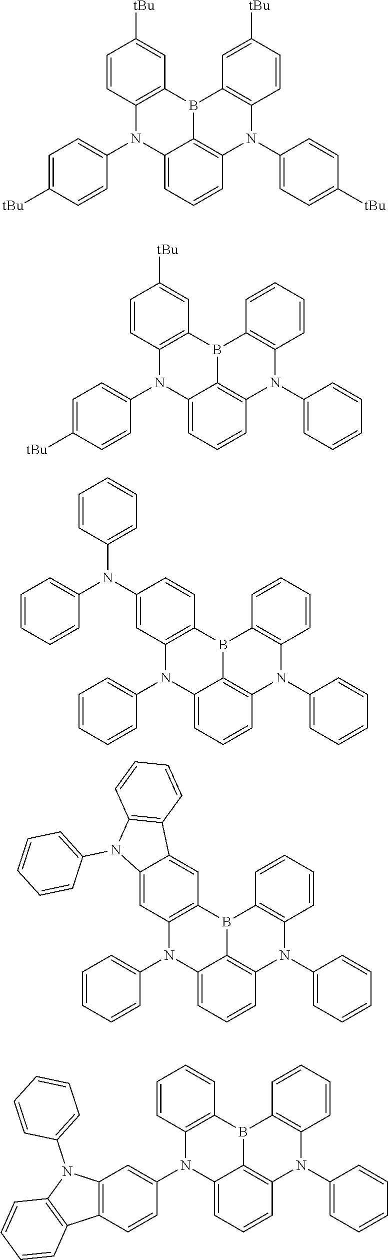

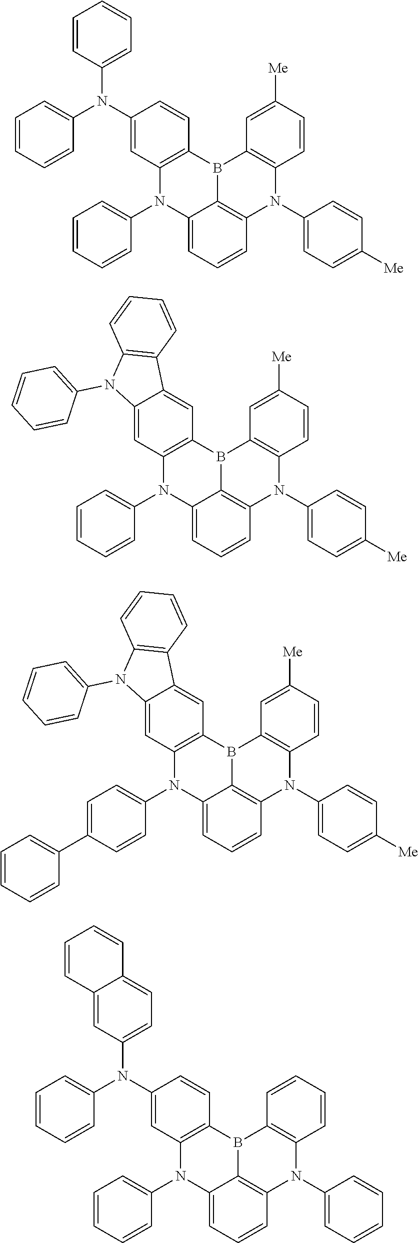

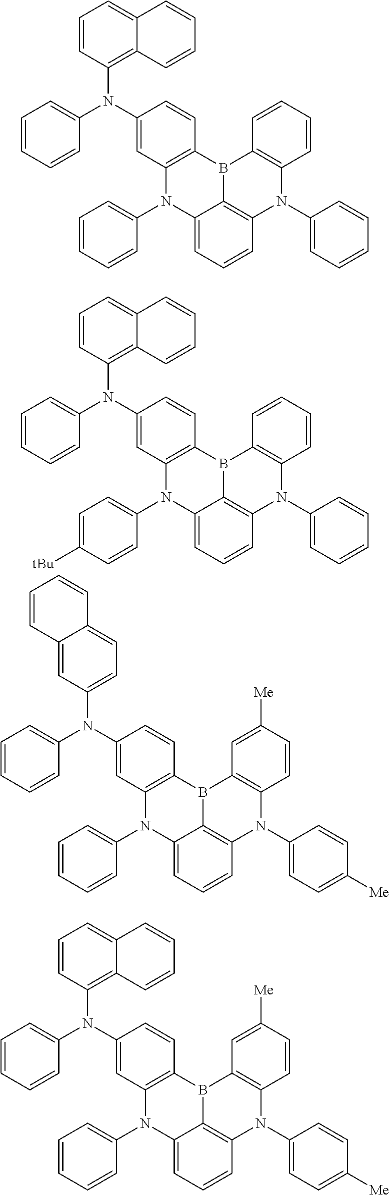

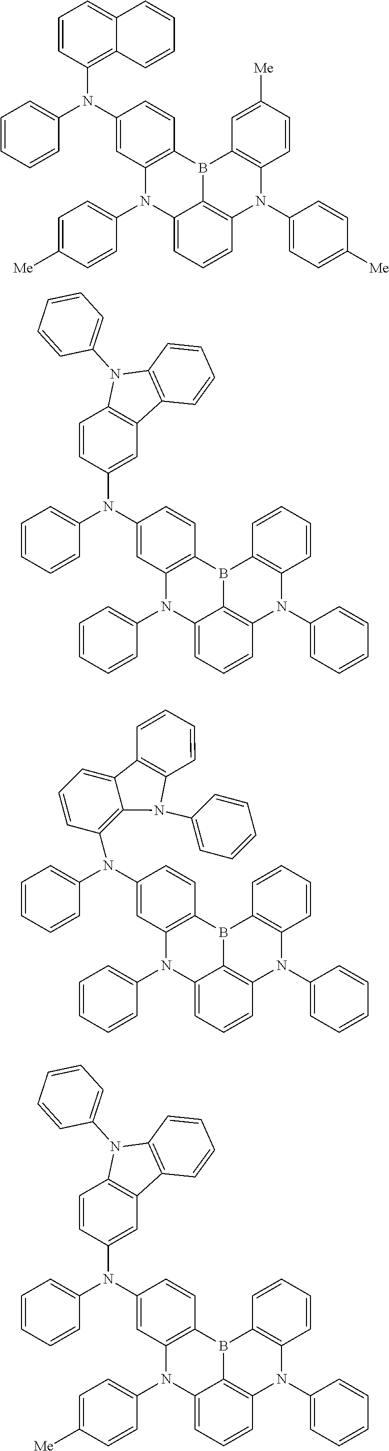

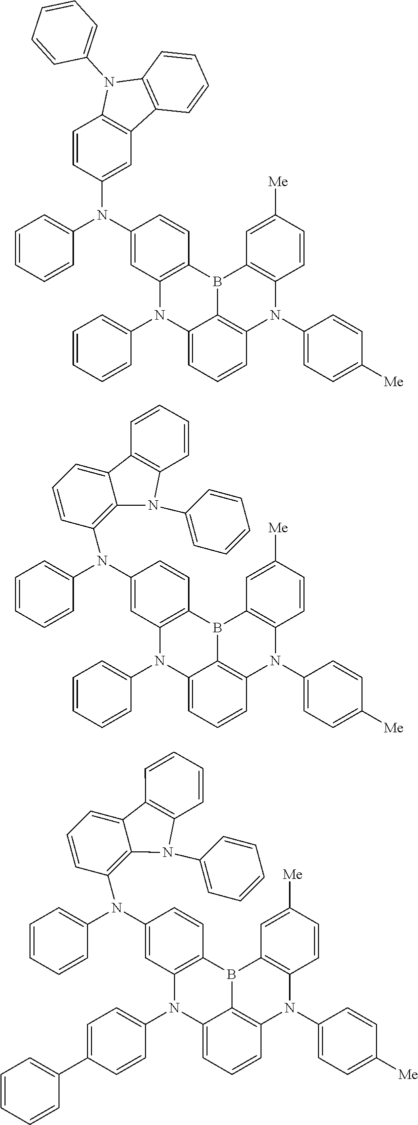

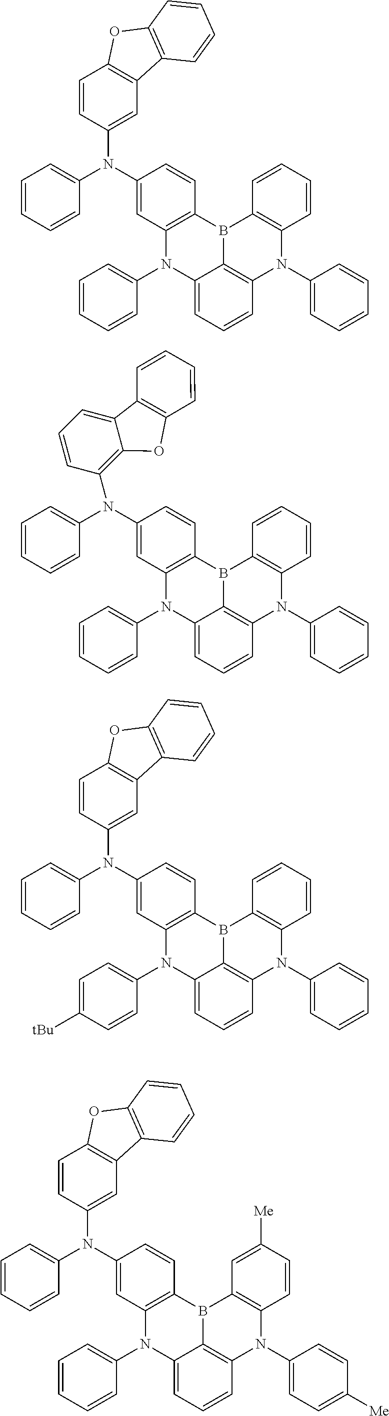

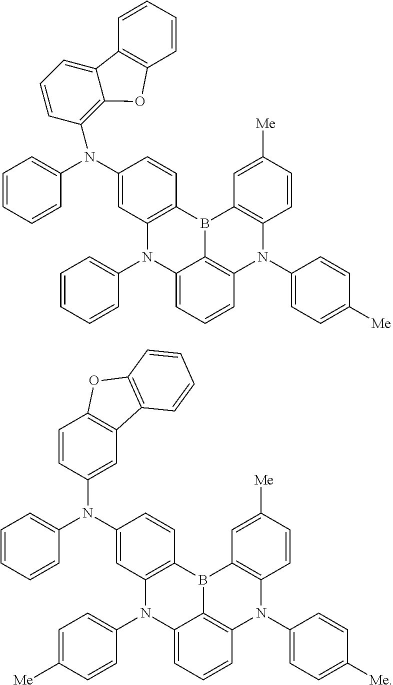

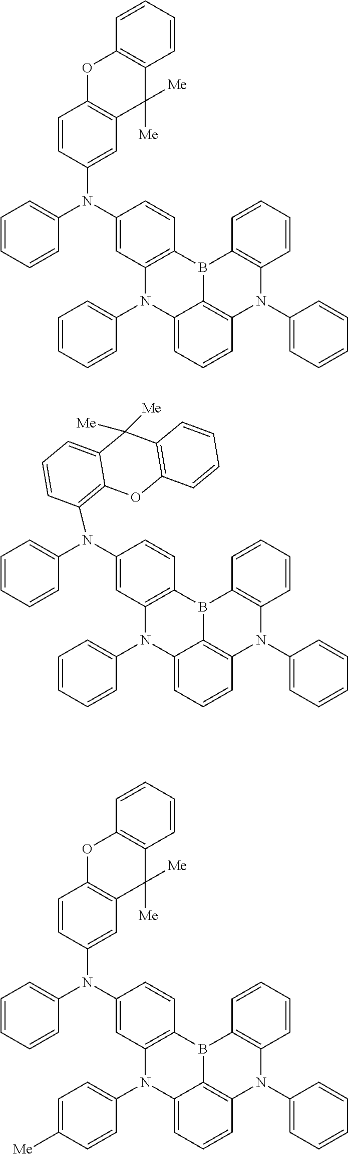 [SMILES: CC1(C)c2ccccc2Oc2ccc(N(c3ccccc3)c3ccc4c(c3)N(c3ccccc3)c3cccc5c3B4c3ccccc3N5c3ccccc3)cc21 ; CC1(C)c2ccccc2Oc2c(N(c3ccccc3)c3ccc4c(c3)N(c3ccccc3)c3cccc5c3B4c3ccccc3N5c3ccccc3)cccc21 ; Cc1ccc(N2c3cc(N(c4ccccc4)c4ccc5c(c4)C(C)(C)c4ccccc4O5)ccc3B3c4ccccc4N(c4ccccc4)c4cccc2c43)cc1]

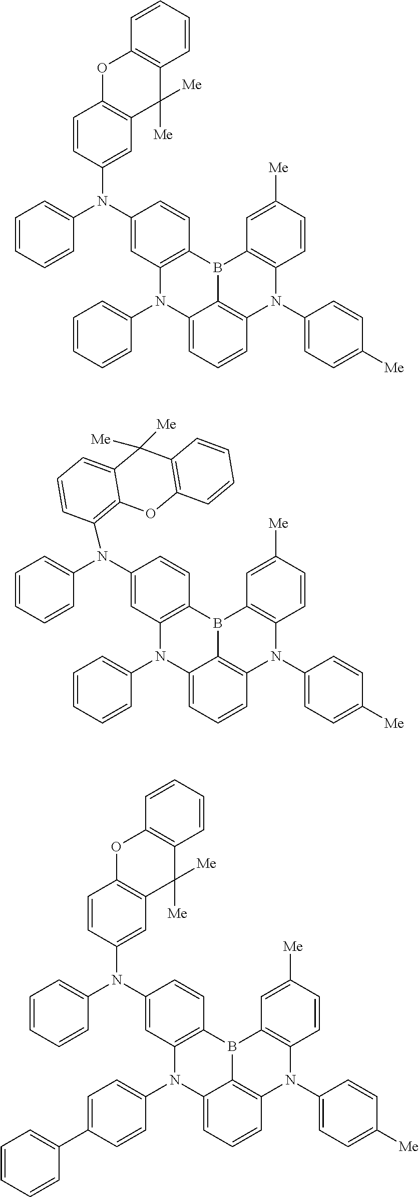 [SMILES: Cc1ccc(N2c3ccc(C)cc3B3c4ccc(N(c5ccccc5)c5ccc6c(c5)C(C)(C)c5ccccc5O6)cc4N(c4ccccc4)c4cccc2c43)cc1 ; Cc1ccc(N2c3ccc(C)cc3B3c4ccc(N(c5ccccc5)c5cccc6c5Oc5ccccc5C6(C)C)cc4N(c4ccccc4)c4cccc2c43)cc1 ; Cc1ccc(N2c3ccc(C)cc3B3c4ccc(N(c5ccccc5)c5ccc6c(c5)C(C)(C)c5ccccc5O6)cc4N(c4ccc(-c5ccccc5)cc4)c4cccc2c43)cc1]

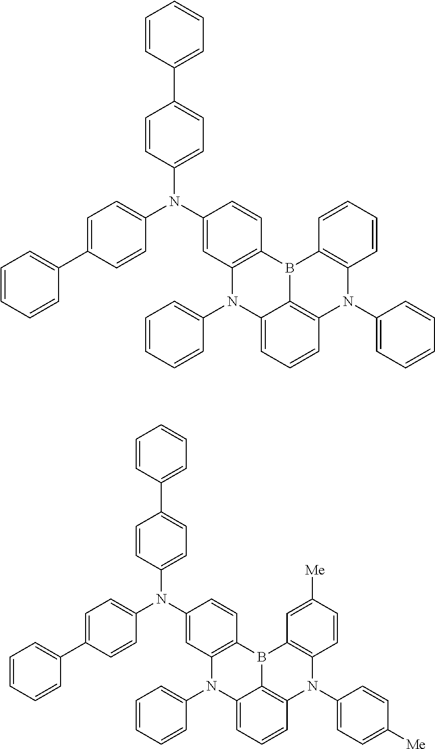 [SMILES: c1ccc(-c2ccc(N(c3ccc(-c4ccccc4)cc3)c3ccc4c(c3)N(c3ccccc3)c3cccc5c3B4c3ccccc3N5c3ccccc3)cc2)cc1 ; Cc1ccc(N2c3ccc(C)cc3B3c4ccc(N(c5ccc(-c6ccccc6)cc5)c5ccc(-c6ccccc6)cc5)cc4N(c4ccccc4)c4cccc2c43)cc1]

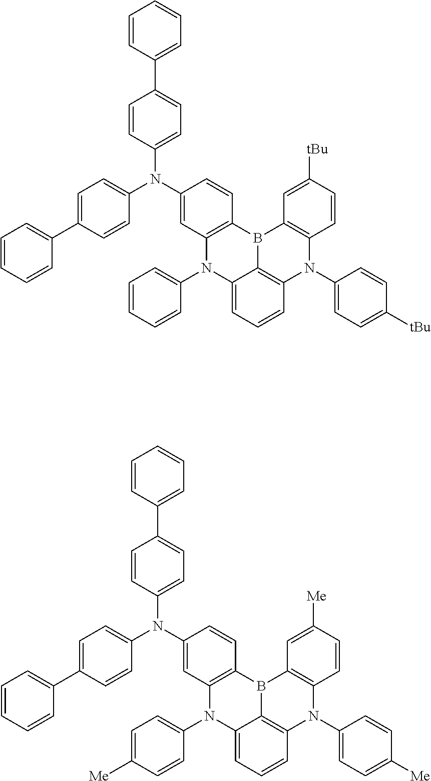 [SMILES: CC(C)(C)c1ccc(N2c3ccc(C(C)(C)C)cc3B3c4ccc(N(c5ccc(-c6ccccc6)cc5)c5ccc(-c6ccccc6)cc5)cc4N(c4ccccc4)c4cccc2c43)cc1 ; Cc1ccc(N2c3ccc(C)cc3B3c4ccc(N(c5ccc(-c6ccccc6)cc5)c5ccc(-c6ccccc6)cc5)cc4N(c4ccc(C)cc4)c4cccc2c43)cc1]

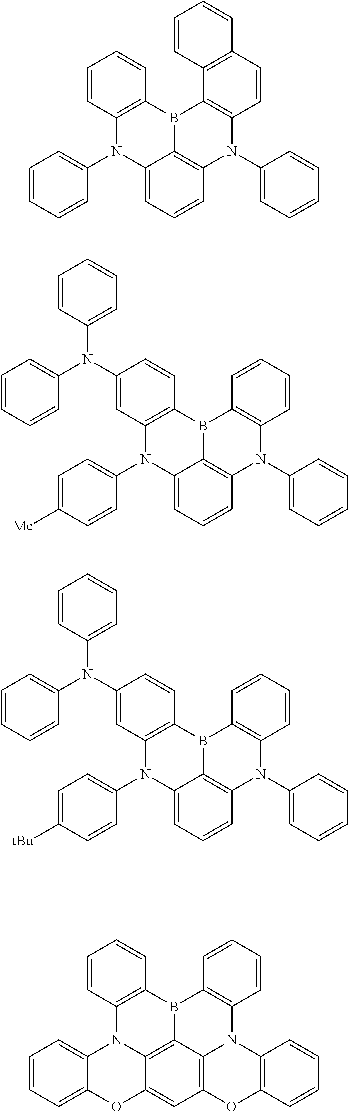 [SMILES: c1ccc(N2c3ccccc3B3c4c2cccc4N(c2ccccc2)c2ccc4ccccc4c23)cc1 ; Cc1ccc(N2c3cc(N(c4ccccc4)c4ccccc4)ccc3B3c4ccccc4N(c4ccccc4)c4cccc2c43)cc1 ; CC(C)(C)c1ccc(N2c3cc(N(c4ccccc4)c4ccccc4)ccc3B3c4ccccc4N(c4ccccc4)c4cccc2c43)cc1 ; c1ccc2c(c1)B1c3ccccc3-n3c4ccccc4oc4cc5oc6ccccc6n-2c5c1c43]

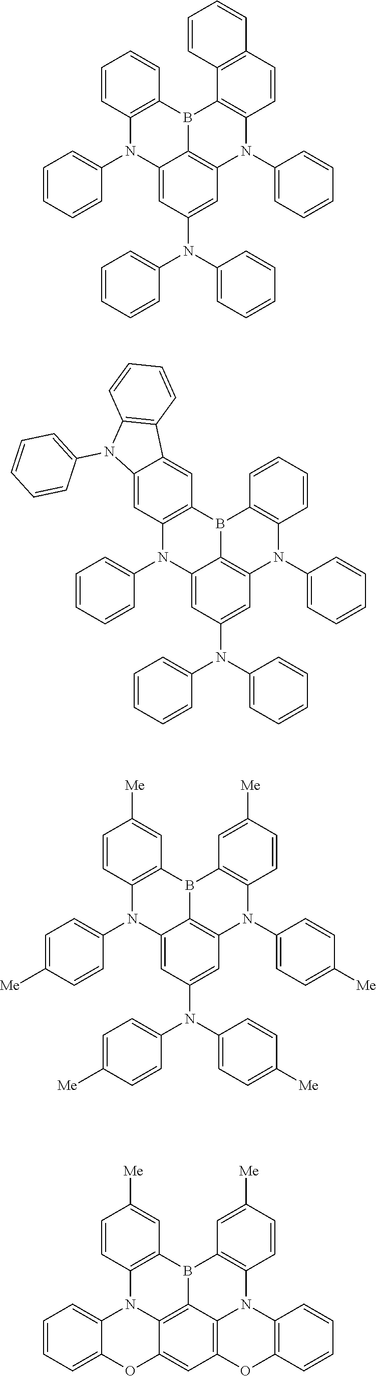

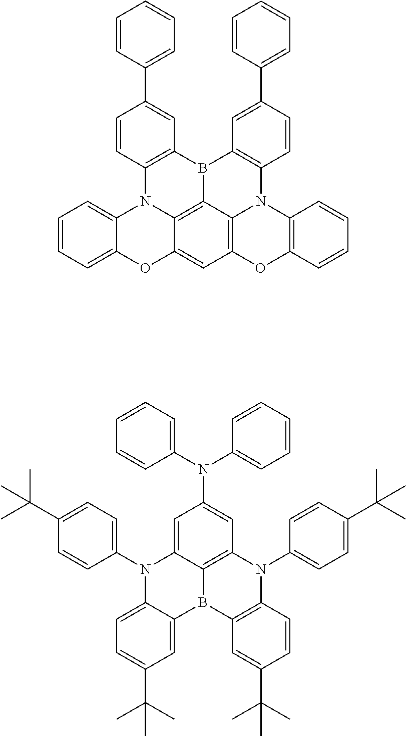 [SMILES: c1ccc(-c2ccc3c(c2)B2c4cc(-c5ccccc5)ccc4-n4c5ccccc5oc5cc6oc7ccccc7n-3c6c2c54)cc1 ; CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5ccccc5)cc2c43)cc1]

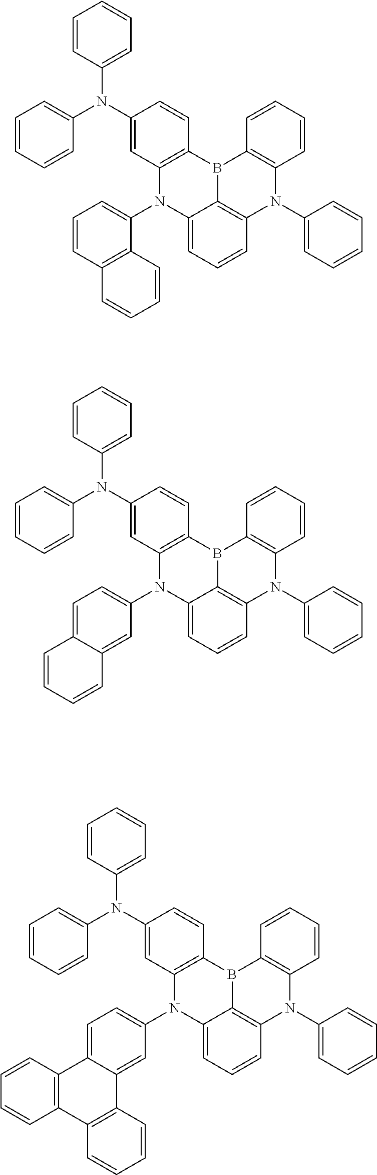 [SMILES: c1ccc(N(c2ccccc2)c2ccc3c(c2)N(c2cccc4ccccc24)c2cccc4c2B3c2ccccc2N4c2ccccc2)cc1 ; c1ccc(N(c2ccccc2)c2ccc3c(c2)N(c2ccc4ccccc4c2)c2cccc4c2B3c2ccccc2N4c2ccccc2)cc1 ; c1ccc(N(c2ccccc2)c2ccc3c(c2)N(c2ccc4c5ccccc5c5ccccc5c4c2)c2cccc4c2B3c2ccccc2N4c2ccccc2)cc1]

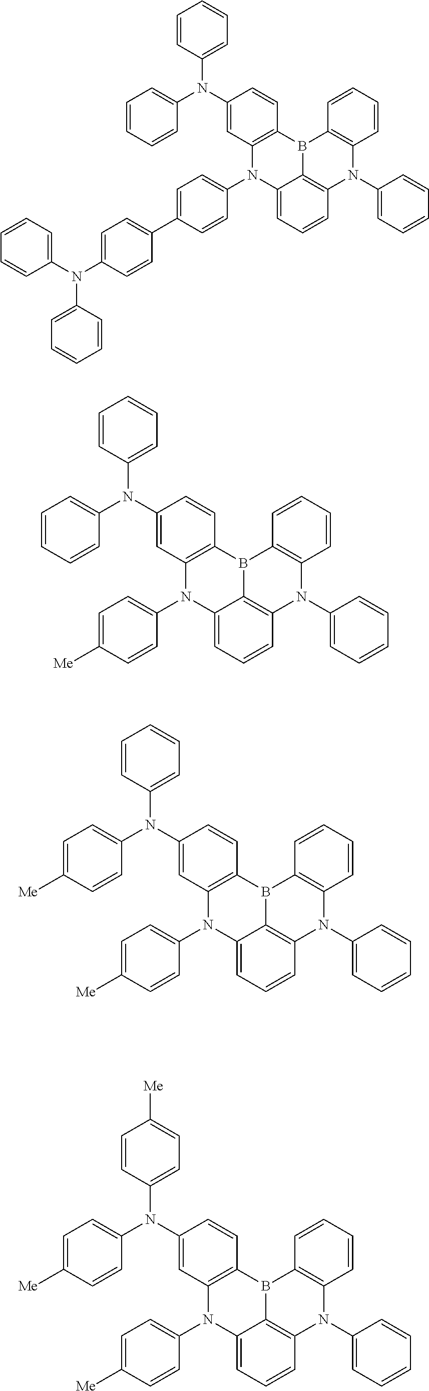 [SMILES: c1ccc(N(c2ccccc2)c2ccc(-c3ccc(N4c5cc(N(c6ccccc6)c6ccccc6)ccc5B5c6ccccc6N(c6ccccc6)c6cccc4c65)cc3)cc2)cc1 ; Cc1ccc(N2c3cc(N(c4ccccc4)c4ccccc4)ccc3B3c4ccccc4N(c4ccccc4)c4cccc2c43)cc1 ; Cc1ccc(N(c2ccccc2)c2ccc3c(c2)N(c2ccc(C)cc2)c2cccc4c2B3c2ccccc2N4c2ccccc2)cc1 ; Cc1ccc(N(c2ccc(C)cc2)c2ccc3c(c2)N(c2ccc(C)cc2)c2cccc4c2B3c2ccccc2N4c2ccccc2)cc1]

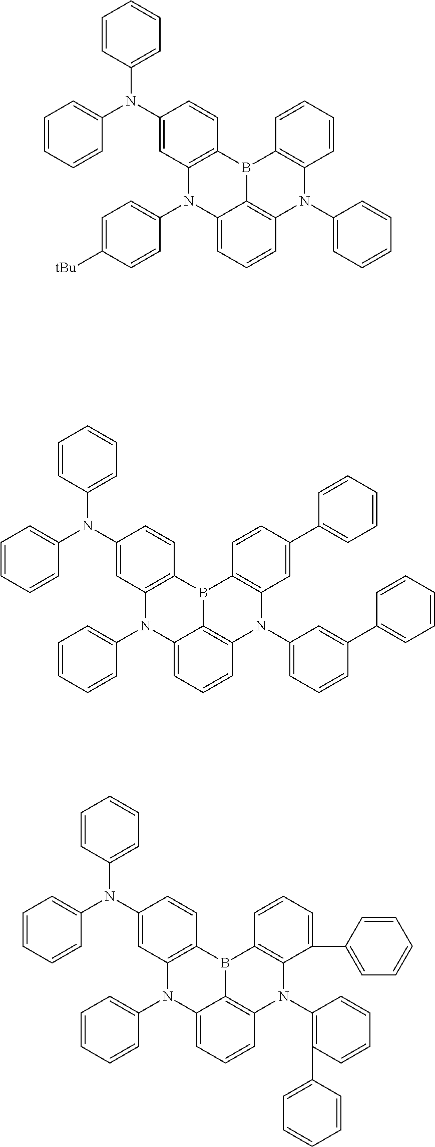 [SMILES: CC(C)(C)c1ccc(N2c3cc(N(c4ccccc4)c4ccccc4)ccc3B3c4ccccc4N(c4ccccc4)c4cccc2c43)cc1 ; c1ccc(-c2cccc(N3c4cc(-c5ccccc5)ccc4B4c5ccc(N(c6ccccc6)c6ccccc6)cc5N(c5ccccc5)c5cccc3c54)c2)cc1 ; c1ccc(-c2ccccc2N2c3cccc4c3B(c3ccc(N(c5ccccc5)c5ccccc5)cc3N4c3ccccc3)c3cccc(-c4ccccc4)c32)cc1]

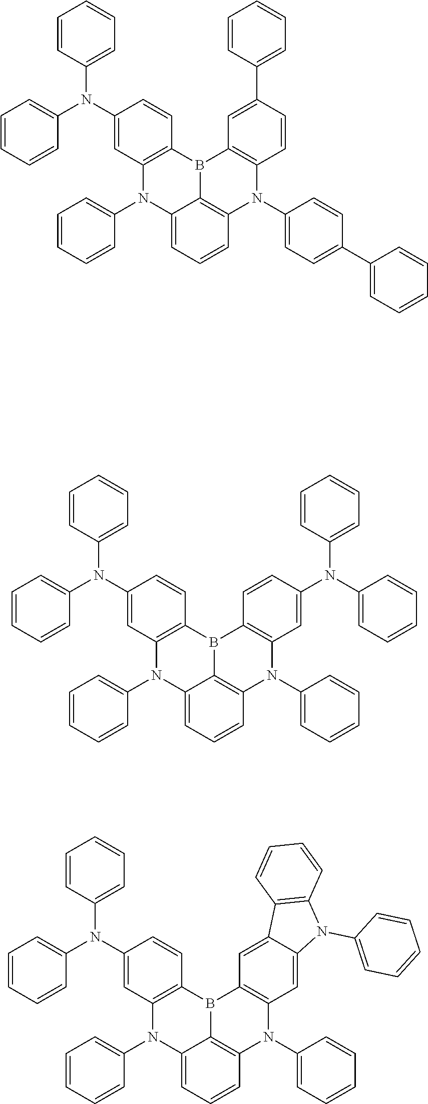 [SMILES: c1ccc(-c2ccc(N3c4ccc(-c5ccccc5)cc4B4c5ccc(N(c6ccccc6)c6ccccc6)cc5N(c5ccccc5)c5cccc3c54)cc2)cc1 ; c1ccc(N(c2ccccc2)c2ccc3c(c2)N(c2ccccc2)c2cccc4c2B3c2ccc(N(c3ccccc3)c3ccccc3)cc2N4c2ccccc2)cc1 ; c1ccc(N(c2ccccc2)c2ccc3c(c2)N(c2ccccc2)c2cccc4c2B3c2cc3c5ccccc5n(-c5ccccc5)c3cc2N4c2ccccc2)cc1]

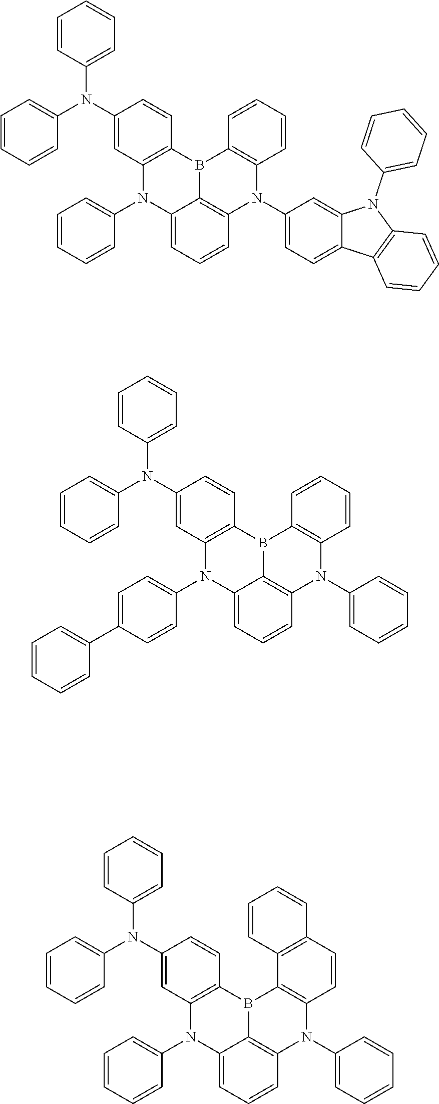 [SMILES: c1ccc(N(c2ccccc2)c2ccc3c(c2)N(c2ccccc2)c2cccc4c2B3c2ccccc2N4c2ccc3c4ccccc4n(-c4ccccc4)c3c2)cc1 ; c1ccc(-c2ccc(N3c4cc(N(c5ccccc5)c5ccccc5)ccc4B4c5ccccc5N(c5ccccc5)c5cccc3c54)cc2)cc1 ; c1ccc(N(c2ccccc2)c2ccc3c(c2)N(c2ccccc2)c2cccc4c2B3c2c(ccc3ccccc23)N4c2ccccc2)cc1]

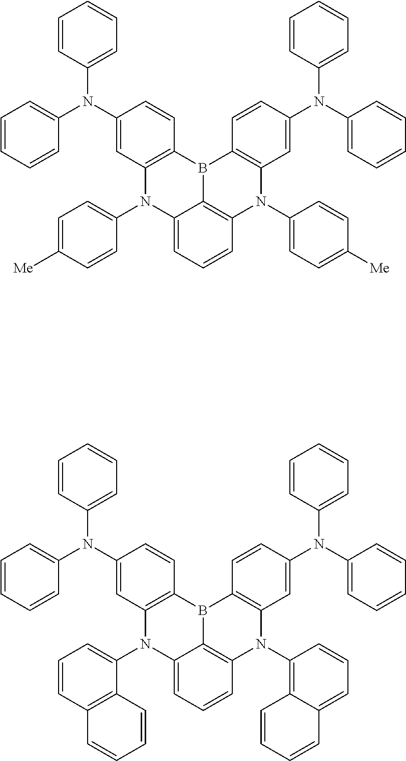 [SMILES: Cc1ccc(N2c3cc(N(c4ccccc4)c4ccccc4)ccc3B3c4ccc(N(c5ccccc5)c5ccccc5)cc4N(c4ccc(C)cc4)c4cccc2c43)cc1 ; c1ccc(N(c2ccccc2)c2ccc3c(c2)N(c2cccc4ccccc24)c2cccc4c2B3c2ccc(N(c3ccccc3)c3ccccc3)cc2N4c2cccc3ccccc23)cc1]

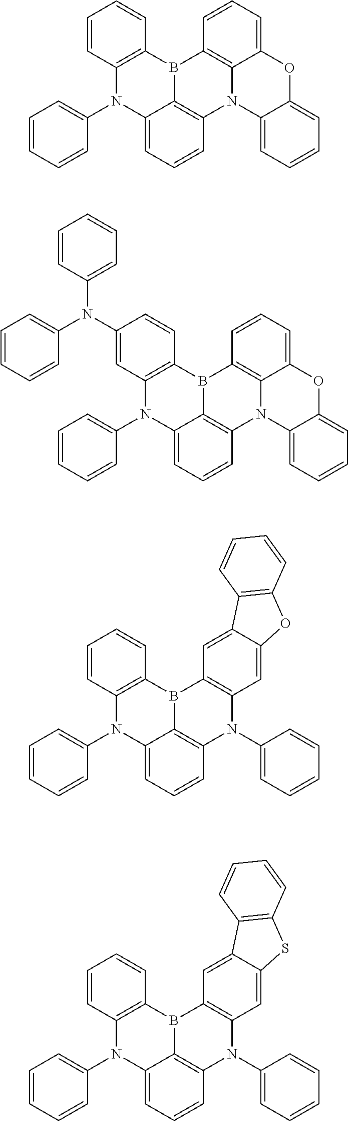 [SMILES: c1ccc(N2c3ccccc3B3c4cccc5c4N(c4ccccc4O5)c4cccc2c43)cc1 ; c1ccc(N(c2ccccc2)c2ccc3c(c2)N(c2ccccc2)c2cccc4c2B3c2cccc3c2N4c2ccccc2O3)cc1 ; c1ccc(N2c3ccccc3B3c4cc5c(cc4N(c4ccccc4)c4cccc2c43)oc2ccccc25)cc1 ; c1ccc(N2c3ccccc3B3c4cc5c(cc4N(c4ccccc4)c4cccc2c43)sc2ccccc25)cc1]

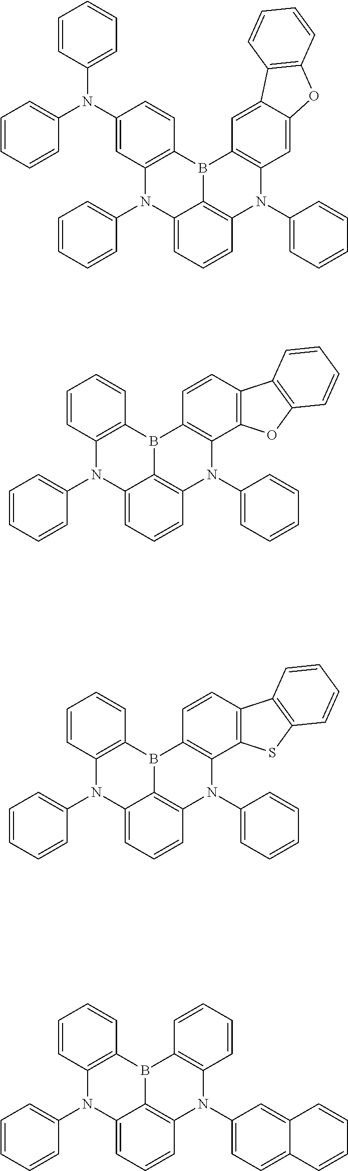 [SMILES: c1ccc(N(c2ccccc2)c2ccc3c(c2)N(c2ccccc2)c2cccc4c2B3c2cc3c(cc2N4c2ccccc2)oc2ccccc23)cc1 ; c1ccc(N2c3ccccc3B3c4ccc5c(oc6ccccc65)c4N(c4ccccc4)c4cccc2c43)cc1 ; c1ccc(N2c3ccccc3B3c4ccc5c(sc6ccccc65)c4N(c4ccccc4)c4cccc2c43)cc1 ; c1ccc(N2c3ccccc3B3c4ccccc4N(c4ccc5ccccc5c4)c4cccc2c43)cc1]

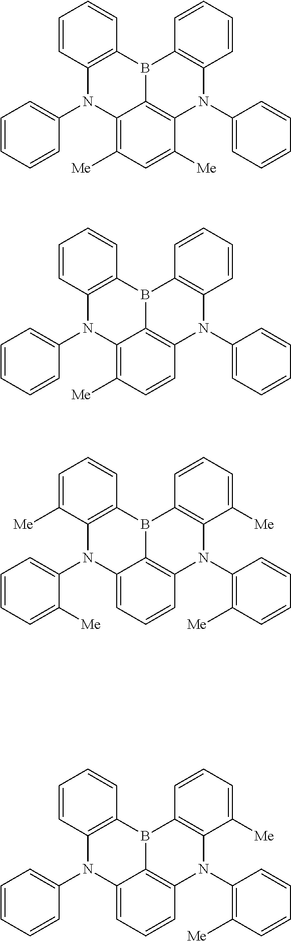 [SMILES: Cc1cc(C)c2c3c1N(c1ccccc1)c1ccccc1B3c1ccccc1N2c1ccccc1 ; Cc1ccc2c3c1N(c1ccccc1)c1ccccc1B3c1ccccc1N2c1ccccc1 ; Cc1ccccc1N1c2cccc3c2B(c2cccc(C)c21)c1cccc(C)c1N3c1ccccc1C ; Cc1ccccc1N1c2cccc3c2B(c2ccccc2N3c2ccccc2)c2cccc(C)c21]

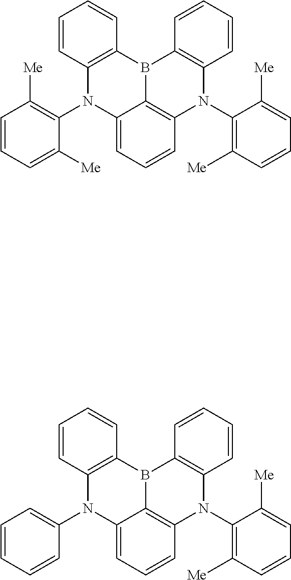 [SMILES: Cc1cccc(C)c1N1c2ccccc2B2c3ccccc3N(c3c(C)cccc3C)c3cccc1c32 ; Cc1cccc(C)c1N1c2ccccc2B2c3ccccc3N(c3ccccc3)c3cccc1c32]

### <Properties of First Component and Second Component, and the Like>

In one embodiment, the first component and the second component are organic semiconductor materials, and for example, they are materials for an organic EL device, materials for an organic transistor, or materials for an organic solar cell.

Examples of materials for an organic EL device include an emitting compound (dopent material for an emitting layer), an organic compound for dispersing an emitting compound and transporting charge to the emitting compound (host material for an emitting layer), a hole-transporting compound, an electron-transporting compound, and the like.

Examples of the dopant material for an emitting layer include a fluorescent emitting compound which emits fluorescence and a phosphorescent emitting compound which emits phosphorescence, and examples of the emitting light colors include blue, green, and red. In one embodiment, examples of the dopant material include a blue phosphorescent emitting compound.

The host material for an emitting layer is preferably an organic compound suitable for the above dopant material, and examples thereof include a host material for a fluorescent emitting device, a host material for a blue phosphorescent emitting device, and the like.

The compounds represented by the formulas (2) and (H11) to (H16) can be used as the host material for an emitting layer, and the compounds represented by the formulas (5) and (6) can be used as the dopant material for an emitting layer.

In one embodiment, the first component is the host material for an emitting layer, and the second component is the dopant material for an emitting layer.

In one embodiment, the first component is the host material for a fluorescent emitting layer, and the second component is the dopant material (fluorescent emitting compound) for a fluorescent emitting layer.

In one embodiment, the first component is a hole-injecting compound, and the second component is a hole-transporting compound.

In one embodiment, the first component is the host material for a fluorescent emitting layer, and the second component is the host material for a fluorescent emitting layer.

In one embodiment, the mixed powder does not include the phosphorescent emitting compound.

In one embodiment, the mixed powder does not include a heavy metal complex.

In one embodiment, the second component is a fluorescent emitting compound which emits fluorescence with a main peak wavelength of 500 nm or less. A “blue fluorescent emitting material” described later in the [Method for fabricating organic EL device] can be used as the compound.

The method for measuring the main peak wavelength of the compound is as follows. 5 μmol/L of a toluene solution of compound being a measurement target is prepared as a sample, it is put in a quartz cell, and then an emission spectrum of the sample is measured at room temperature (300 K) (as a vertical axis: emission strength and a horizontal axis: wavelength). The emission spectrum can be measured using a fluorescence spectrophotometer (apparatus name: F-7000) manufactured by Hitachi High-Tech Science Corporation. The main peak wavelength in the emission spectrum means a peak wavelength of the emission spectrum with maximum emission strength.

In one embodiment, neither the first component nor the second component is a phosphorescent compound which emits phosphorescence.

In one embodiment, the first component is the host material for an emitting layer, the second component is the dopant material for an emitting layer, and the triplet energy T₁ (H1) of the first component and the triplet energy T₁ (D1) of the second component satisfy the relationship of the following (Numerical formula 1).

$${T}_{1} ( D 1 ) > {T}_{1} ( H 1 ) \qquad ( Numerical formula 1 )$$

The method for measuring the triplet energy T₁ is as follows.

The compound being a measurement target is dissolved in EPA (diethylether:isopentane:ethanol=5:5:2 (volume ratio)) so that the concentration thereof is 10-5 mol/L or more and 10-4 mol/L or less, and this solution is placed in a quartz cell, and it is used as a measurement sample. For this measurement sample, the phosphorescence spectrum (vertical axis: phosphorescent emitting intensity, horizontal axis: wavelength) is measured at low temperature (77 [K]), a tangent line is drawn to the rapidly increasing part on the short wavelength side in this phosphorescence spectrum, the energy amount calculated from the following conversion formula (F1) is defined as triplet energy T₁, using the wavelength value λ_{edge} [nm] at the intersection of the tangent and the horizontal axis.

$${T}_{1} [ eV ] = 1239 \text{.85} / {λ}_{edge} \qquad Conversion formula ( F1 )$$

The tangent line is drawn as follows to the rapidly increasing part on the short wavelength side in this phosphorescence spectrum. Among the maximum values of the spectrum, the tangent line at each point to the long wavelength direction on the curve is considered when moving from the short wavelength side of the phosphorescence spectrum to the maximum value on the shortest wavelength side on the spectrum curve. Regarding this tangent line, the curve is increased (that is, as the vertical value is increased), and then the slope is increased. The tangent line (that is, the tangent line at inflection point) drawn at the point where the value of the slope takes the maximum value is used as the tangent line to the increasing edge on the short wavelength side of the phosphorescence spectrum.

A maximum point with a peak intensity of 15% or less of the maximum peak intensity of the spectrum is not included in the maximum value on the shortest wavelength side mentioned above, and the tangent line closest to the maximum value on the shortest wavelength side and drawn at the point where the slope value takes the maximum value is defined as the tangent line to the rapidly increasing part of the short wavelength side of the phosphorescence spectrum.

The main unit of the Fluorescence Spectrophotometer F-4500 manufactured by Hitachi High-Tech Corporation can be used for measuring phosphorescence. The measurement apparatus is not limited to this, and a cooling apparatus, a low-temperature container, an excitation light source, and a light receiving apparatus may be used in combination to measure it.

The combination of the first component and the second component is not particularly limited as long as the above-described conditions are satisfied, and examples of the combination of the first component and the second component (first component, second component) include (the compound represented by the formula (2), the compound represented by the formula (6)), (the compound represented by the formula (H11), the compound represented by the formula (5)), (the compound represented by the formula (H12), the compound represented by the formula (5)), and the like.

In one embodiment, as the first component and the second component, one or more compounds selected from compounds corresponding to each of the first component and the second component in compounds included in each group of the following (Group 2A) to (Group 35A) can be independently used. The first component and the second component in the present specification are not particularly limited independently to one or more compounds selected from compounds corresponding to each of the first component and the second component in compounds included in each group of the following (Group 2A) to (Group 35A), and they may not be these.

### (Group 2A)

bis(phenylamino)biphenyl; rubrene; bis(phenylamino)biphenyl; TBP; OP31; tris(quinolinolato)aluminum; coumarin545T

### (Group 4A)

Alq3; NPB; TBADN; DCJTP; rubrene; OP31; DPQA; DBZR

### (Group 5A)

tris(methylphenylphenylamino)triphenylamine; tetrafluorotetracyanoquinodimethane; bathophenanthroline; tris(carbazolyl)triphenylamine; fac-tris(phenylpyridine)iridium; zinc phthalocyanine; fullerene C60

### (Group 6A)

tris(methylphenylphenylamino)triphenylamine; tetrafluorotetracyanoquinodimethane; bathophenanthroline; tris(carbazolyl)triphenylamine; fac-tris(phenylpyridine)iridium; zinc phthalocyanine; fullerene C60

### (Group 9A)

quatemaphthalene; (binaphthalenyl)(naphthyl)benzene; ternaphthalene; (binaphthalenyl)(naphthyl)benzene; (binaphthalenyl)(naphthyl)benzene; quaternaphthalene; quaternaphthalene; quaternaphthalene; quaternaphthalene; (ternaphthalenyl)(naphthyl)benzene; [bis(isopropylnaphthalenyl)]biphenyl; [(ternaphthalenyl)]biphenyl; (ternaphthalenyl)(naphthyl)benzene; (ternaphthalenyl)(naphthyl)benzene; diphenylphenanthrene; tetraphenylphenanthrene; dimethylbis(methylphenyl)phenanthrene; bis(trifluoromethyl)bis(trifluoromethylphenyl)phenanthrene; dimethoxybis(methoxyphenyl)phenanthrene; diphenylbis(biphenylyl)phenanthrene; diphenylbis(methylphenyl)phenanthrene; dimethyl(biphenylyl)phenanthrene; methylphenylmethylphenyl(biphenylyl)phenanthrene; di(naphthyl)phenanthrene; di(pyridyl)phenanthrene; di(tertbutyl)diphenylphenanthrene; distyrylphenanthrene; cyanophenanthrene; bis(diphenylvinyl)phenanthrene; (binaphthalenyl)(phenanthrenyl)benzene; di(phenanthrenyl)naphthalene; bis(diphenylphenanthrenyl)naphthalene; [di(phenanthrenyl)]binaphthalene; (binaphthalenyl)phenanthrene; di(phenanthrenyl)benzene; di(phenanthrenyl)naphthalene; (naphthyl)(phenanthrenyl)phenanthrene; (naphthyl)(phenanthrenyl)phenanthrene; diphenyl(naphthyl)(phenanthrenyl)phenanthrene; (naphthyl)(phenanthrenyl)diphenylphenanthrene; phenyl(phenanthrenyl)naphthalene; diphenyldi(phenanthrenyl)naphthalene; naphthyl(phenanthrenylphenyl)phenanthrene; [di(phenanthrenyl)]biphenyl; (naphthyl)di(phenanthrenyl)benzene; (naphthyl)di(phenanthrenyl)benzene; tris(phenanthrenyl)naphthalene; [(phenyl)naphthalenyl]phenylphenanthrene; [(phenyl)naphthalenyl](naphthyl)phenanthrene; (binaphthalenyl)(naphthyl)phenanthrene; (binaphthalenyl)(phenanthrenyl)phenanthrene; [di(naphthyl)]biphenanthrene; (naphthyl)terphenanthrene; (phenylphenanthrenyl)(phenanthrenyl)benzene; (diphenylbinaphthalenyl)(naphthyl)phenanthrene; [di(naphthyl)diphenyl]biphenanthrene; [di(naphthyl)diphenyl]biphenanthrene; (binaphthalenyl)(naphthyl)phenanthrene; [(naphthyl)phenyl](naphthyl)phenanthrene; [(binaphthalenyl)(phenanthrenyl)biphenyl; [(phenanthrenyl)phenyl](naphthylphenyl)phenanthrene; bis[(phenanthrenyl)phenyl]naphthalene; bis[(phenanthrenyl)phenyl]naphthalene; bis[(phenanthrenyl)phenyl]naphthalene; bis[(phenanthrenyl)phenyl]naphthalene; bis[(phenanthrenyl)phenyl]phenanthrene; bis[(phenanthrenyl)phenyl]naphthalene; bis[(phenanthrenyl)phenyl]naphthalene; bis[(phenanthrenyl)phenyl]naphthalene; (binaphthalenyl[(biphenylyl)phenanthrenyl]benzene; (naphthyl[(phenanthrenyl)naphthalenyl]benzene; [(phenanthrenyl)naphthalenyl](phenanthrenyl)benzene; (binaphthalenyl)(phenanthrenyl)benzene; (binaphthalenyl)(phenanthrenyl)benzene; [(phenanthrenylnaphthalenyl](phenanthrenyl)benzene; (binaphthalenyl)(phenanthrenyl)benzene; (binaphthalenyl)(phenanthrenyl)benzene; [(phenanthrenylnaphthalenyl](phenanthrenyl)benzene; (binaphthalenyl)(phenanthrenyl)benzene; (binaphthalenyl)(phenanthrenyl)benzene; (binaphthalenyl)(phenanthrenyl)benzene; [(phenanthrenylnaphthalenyl](phenanthrenyl)benzene; [(phenylnaphthalenyl](phenanthrenyl)benzene; ethyl[ethyl(binaphthalenyl)](phenanthrenyl)benzene; [(phenyl)binaphthalenyl]phenanthrene; [bis(phenanthrenyl)]binaphthalene; (benzophenanthrenyl(phenanthrenyl)binaphthalene; (benzochrysenyl){[(tertbutyldimethylsilyl)phenanthrenyl]naphthalenyl}benzene; [phenyl(phenanthrenyl)]binaphthalene; (phenanthrenylphenyl)binaphthalene; (ternaphthalenyl)phenanthrene; [bis(phenanthrenyl)]binaphthalene; [bis(phenanthrenyl)]binaphthalene; [(phenanthrenyl)(phenanthrenyl)]binaphthalene; [bis(phenanthrenyl)]binaphthalene; (ternaphthalenyl)phenanthrene; (ternaphthalenyl)phenanthrene; (ternaphthalenyl)phenanthrene; [bis(phenanthrenyl)]binaphthalene; {bis[(trimethylsilyl)phenanthrenyl]}binaphthalene; [bis(phenanthrenyl)]biphenyl; [(naphthyl)(phenanthrenyl)]biphenyl; [(phenanthrenyl)(phenanthrenyl)]biphenyl; [bis(phenanthrenyl)]biphenyl; [bis(phenanthrenyl)]biphenyl; [(phenanthrenyl)(naphthyl)]biphenyl; [dimethylbis(phenanthrenyl)]biphenyl; [(dimethylphenanthrenyl(methylphenanthrenyl)]biphenyl; [bis(diisopropylphenanthrenyl)]biphenyl; [(phenanthrenyl)(trimethylsilylnaphthalenyl)]biphenyl; [difluorobis(phenanthrenyl)]biphenyl; [dicyanobis(phenanthrenyl)]biphenyl; [(phenanthrenyl[(naphthyl)phenyl]binaphthalene; [(biphenylyl(phenanthrenyl)]binaphthalene; {[(phenanthrenyl)phenyl](phenanthrenyl]binaphthalene; {[(phenanthrenyl)phenyl](phenanthrenyl]binaphthalene; {[(phenanthrenyl)phenyl]phenyl]binaphthalene; di(naphthyl)chrysene; di(naphthyl)chrysene; (naphthyl)(naphthyl)chrysene; di(phenanthrenyl)chrysene; (naphthyl)(phenanthrenyl)chrysene; (naphthyl)(phenanthrenyl)chrysene; bis[(naphthyl)phenyl]chrysene; bis[(naphthyl)phenyl]chrysene; [(naphthyl)phenyl][(naphthyl)phenyl]chrysene; (naphthyl[(naphthyl)phenyl]chrysene; [(naphthyl)phenyl](phenylnaphthalenyl)chrysene; [(naphthyl)phenyl](naphthyl)chrysene; bis(phenylnaphthalenyl)chrysene; bis[(biphenylyl)phenyl]chrysene; bis(phenylnaphthalenyl)chrysene; (phenylnaphthalenyl[(phenylnaphthalenyl)phenyl]chrysene; bis[(benzophenanthrenyl)phenyl]chrysene; [(naphthyl)phenyl][(naphthyl)phenyl]chrysene; bis[(naphthyl)phenyl]chrysene; bis[(naphthyl)phenyl]chrysene; phenyl[(phenanthrenyl)naphthalenyl]chrysene; bis(phenylchrysenyl)naphthalene; di(naphthyl)chrysene; (naphthyl)(naphthyl)chrysene; di(naphthyl)chrysene; (naphthyl)(phenanthrenyl)chrysene; di(phenanthrenyl)chrysene; (naphthyl)(phenanthrenyl)chrysene; diphenyldi(naphthyl)chrysene; phenyl(naphthyl)(naphthyl)chrysene; phenyldi(naphthyl)chrysene; phenyl(phenanthrenyl)(naphthyl)chrysene; isopropyldi(phenanthrenyl)chrysene; di(naphthyl)(phenanthrenyl)chrysene; phenyldi(phenanthrenyl)chrysene; (phenanthrenyl)(naphthyl)picene; [(biphenylylnaphthalenyl]chrysene; (chrysenyl[(phenanthrenyl)naphthalenyl]benzene; [(chrysenyl(phenanthrenyl)]binaphthalene; bis(chrysenyl)biphenyl; {phenyl[(cyclohexylchrysenyl)phenyl]binaphthalene; {phenyl[(chrysenyl)phenyl]binaphthalene; (picenyl){(phenanthrenyl)phenyl}naphthalene; diphenylbenzophenanthrene; di(naphthyl)benzophenanthrene; bis(phenylnaphthalenyl)benzophenanthrene; bis(phenylnaphthalenyl)benzophenanthrene; (phenylnaphthalenyl(naphthyl)benzophenanthrene; [(naphthyl)phenyl](naphthyl)benzophenanthrene; bis[(naphthyl)phenyl]benzophenanthrene; bis[(naphthyl)phenyl]benzophenanthrene; (benzophenanthrenyl(phenanthrenyl)benzene; [(naphthyl)phenyl][(naphthyl)phenyl]benzophenanthrene; (benzophenanthrenyl(phenanthrenyl)benzene; diphenylbenzophenanthrene; di(naphthyl)benzophenanthrene; (benzophenanthrenyl(phenanthrenyl)biphenyl; di(naphthyl)benzophenanthrene; (naphthylphenyl(phenanthrenyl)benzophenanthrene; di(naphthyl)benzochrysene; di(naphthyl)benzochrysene; (benzophenanthrenyl(naphthylphenanthrenyl)benzene; (benzochrysenyl(phenylnaphthalenyl)benzene; [(benzochrysenyl(benzophenanthrenyl)]binaphthalene; [(benzophenanthrenyl(naphthyl)]biphenyl; [(benzophenanthrenyl(benzophenanthrenyl)]biphenyl; [(phenanthrenyl)phenyl](benzophenanthrenyl)naphthalene; [(phenanthrenyl)(benzochrysenyl)]binaphthalene; [di(benzochrysenyl)]biphenyl; diphenylbenzochrysene; bis[(naphthyl)phenyl]benzochrysene; bis[(naphthyl)phenyl]benzochrysene; di(naphthyl)benzochrysene; bis(phenylnaphthalenyl)benzochrysene; diphenylhelicene; di(naphthyl)helicene; di(naphthyl)helicene; di(naphthyl)(fluoranthenyl)benzene; diphenyl[(naphthyl)phenyl]fluoranthene; bis[(naphthyl)phenyl]fluoranthene; phenyl(phenanthrenyl)fluoranthene; naphthyltetraphenylfluoranthene; phenanthrenyltetraphenylfluoranthene; diphenyl[(naphthyl)phenyl]fluoranthene; diisopropyldiphenyl[(naphthyl)phenyl]fluoranthene; diphenyl[(phenanthrenyl)phenyl]fluoranthene; di(naphthyl)fluoranthene; (naphthyl)((naphthyl)phenyl)fluoranthene; (naphthylbis[(naphthyl)phenyl]fluoranthene; (naphthyl)(phenanthrenyl)fluoranthene; (naphthyl)(naphthyl)fluoranthene; (biphenylyl(naphthyl)fluoranthene; [(phenylbiphenylyl](naphthyl)fluoranthene; [(phenylbiphenylyl](naphthyl)fluoranthene; (biphenylyl(terbenzenyl)fluoranthene; di(fluoranthenyl)naphthalene; [(benzofluoranthenyl)naphthalenyl][(naphthyl)phenyl]phenanthrene; diphenyldi(fluoranthenyl)naphthalene; {[(benzofluoranthenyl)biphenylyl]}[(benzofluoranthenyl)phenyl]naphthalene; [(benzofluoranthenyl(phenanthrenyl)]biphenyl; [di(fluoranthenyl)]binaphthalene; [(fluoranthenyl)phenyl](phenanthrenyl)naphthalene; [(phenanthrenyl)phenyl](fluoranthenyl)naphthalene; [di(fluoranthenyl)]biphenyl; {[(phenanthrenyl)phenyl](fluoranthenyl)}binaphthalene; (benzofluoranthenyl(phenanthrenyl)benzene; (benzofluoranthenyl(phenanthrenyl)triphenylene; di(naphthyl)(benzofluoranthenyl)benzene; (benzofluoranthenyl(naphthyl)benzene; bis[(naphthyl)phenyl]benzofluoranthene; (phenanthrenyl)benzofluoranthene; tetraphenylbenzofluoranthene; tetraphenyldibenzofluoranthene; diphenyl[(naphthyl)phenyl]benzofluoranthene; diphenyl[(naphthyl)phenyl]benzofluoranthene; diphenyl[(phenanthrenyl)phenyl]benzofluoranthene; diphenylbenzofluoranthene; bis[(phenyl)biphenylyl]diphenylbenzofluoranthene; (phenyldiphenylbenzofluoranthene; (diphenylbenzofluoranthenylphenylnaphthalene; di(naphthyldiphenylbenzofluoranthene; (biphenylyldiphenyl[(naphthyl)phenyl]benzofluoranthene; bis(biphenylyldiphenylbenzofluoranthene; di(methylstyryl)triphenylene; distyryltriphenylene; tri(methylstyryl)triphenylene; tristyryltriphenylene; diphenyltriphenylene; (naphthylphenyltriphenylene; diphenyltriphenylene; hexaphenyltriphenylene; triphenyltriphenylene; tris(biphenylyl)triphenylene; tetraphenyltriphenylene; (triphenylenyl(phenanthrenyldiphenylphenanthrene; naphthyl(phenanthrenyl)triphenylene; (biphenanthrenyl)triphenylene; bis(triphenylenyl)binaphthalene; [(triphenylenyl)phenyl](phenanthrenyl)naphthalene; [bis(triphenylenyl)]biphenyl; [bis(triphenylenyl)]biphenyl; {[(triphenylenyl)]phenylphenyl}binaphthalene; [(naphthyl)phenyl][(naphthyl)phenyl]naphthoquinoline; bis(naphthylazabenzochrysene; diphenyldiazodibenzophenanthrene; bis(phenylnaphthalenyl)acenaphthopyrazine; bis(phenylnaphthalenyldiazabenzofluoranthene; diphenylazabenzofluoranthene; iridium(III)bis(phenylquinolyl)acetylacetonate; iridium(III)tris(phenylisoquinolyl)iridium(III)bis((tertbutylphenyl)isoquinolyl)acetylacetonate; iridium(III)bis(diphenylquinoxalyl)acetylacetonate; iridium(III)bis(diphenyltetrahydroquinoxalyl)acetylacetonate; iridium(III)bis(diphenylpyrazine)acetylacetonate; osmium(IV)bis[(triphenylmethyltriazolylmethylpyridyl]bis(methyldiphenylphosphonate); ruthenium(V)bis[dipyridyl](phenylpyridyl); platinum(II)bis[(thienyl)pyridyl]; iridium(III)bis[(benzothiophenyl]pyridine]acetylacetonate; iridium(III)bis(phenylbenzothiazole)acetylacetonate; iridium(III)tris(diphenylquinoxalyl); iridium(III)bis(phenylphenantridyl)acetylacetonate; iridium(III)bis((biphenylyl)pyridine)acetylacetonate; iridium(III)bis(phenylisoquinolyl)(acetylhydroxycyclohexenone); platinum(II)bis[phenylenebis(ditertbutylsalicylideneamino]; iridium(III)tris(difluorodibenzoquinolyl)acetylacetonate; platinum(II)(octaethylporphyrin); iridium(III)tris[(fluoro)phenylpyridyl]; iridium(III)bis[(fluoro)phenylpyridyl]acetylacetonate; iridium(III)tris[(difluorophenyl)pyridyl]; iridium(III)tris[(fluorophenylmethoxypyridyl]; iridium(III)bis[(fluorophenyl)pyridyl][(difluorophenyl)pyridyl]; iridium(III)tris[(difluorophenylmethoxypyridyl]; iridium(III)tris[(fluorophenyldiphenylaminopyridyl]; iridium(III)tris[(fluorophenyldimethylaminopyridyl]acetylacetonate; iridium(III)tris[(difluorophenylmethylpyridyl]; platinum(II)[di(pyridyl)benzil](biphenylone]; iridium(III)tris[(methylphenylpyridyl]; iridium(III)tris[(methylphenylpyridyl]; iridium(III)bis[phenylisoquinolyl]acetylacetonate; iridium(III)bis[phenylpyridyl]acetylacetonate; iridium(III)bis[(bis(trifluoromethyl)phenyl)pyridyl)(carboxylatepyridiniu); iridium(III)bis[(difluorophenyl)pyridyl)(carboxylatepyridiniu); iridium(III)bis[(difluorophenyl)pyridyl)[(trifluoromethyl)(pyridinyltriazole); iridium(III)tris[phenylpyridyl); iridium(III)bis[phenylisoquinolyl]acetylacetonate; iridium(III)bis[(difluorophenyl)pyridyl)[tetrakis(Hpyrazolyl)boron]; iridium(III)tris[phenylmethyldihydrobenzimidazole]; iridium(III)tris[(dibenzofuranylmethylimidazoline]; iridium(III)tris[(dimethylfluorenylpyrazole]; platinum(II)bis(phenylpyridine); iridium(III)tris[(trifluorophenyl)pyridyl]; iridium(III)bis[(thienyl)pyridyl]acetylacetonate; iridium(III)bis(phenylbenzoxazole)acetylacetonate; iridium(III)bis[(naphthyl)benzoxazole]acetylacetonate; iridium(III)bis[(benzothiazolyldimethylaminocoumaryl]acetylacetonate; iridium(III)bis[(naphthyl)isoquinolyl]acetylacetonate; iridium(III)bis[(naphthyl)isoquinolyl]acetylacetonate; iridium(III)bis[(pyridyl)isoquinolyl]acetylacetonate; iridium(III)bis(phenylisoquinolyl)(phenylpyridyl); iridium(III)tris[(thienyl)isoquinolyl]

### (Group 10A)

[di(carbazolyl)]biphenyl; [dimethyldi(carbazolyl)]biphenyl; [diphenyldi(carbazolyl)]biphenyl; di(carbazolyl)benzene; tri(carbazolyl)benzene; di(carbazolyl)triphenylsilylbenzene; tris[(carbazolyl)phenyl]amine; tris[(carbazolyl)phenyl]benzene; tris{[methylphenyl(phenyl)amino]phenyl}amine; tris{[naphthyl(phenyl)amino]phenyl}amine; [bis(tribenzazepinyl)]biphenyl; [dimethylbis(tribenzazepinyl)]biphenyl; bis(tribenzazepinyl)benzene; tris(tribenzazepinyl)benzene; tris[(tribenzazepinyl)phenyl]amine; tris[(tribenzazepinyl)phenyl]benzene; diphenylbis[(carbazolyl)phenyl]silane; diphenylbis[(carbazolyl)phenyl]methane; di(carbazolyl)quaterbenzene; di(carbazolyl)quaterbenzene; di(carbazolyl)quaterbenzene; bis[(carbazolyl)phenyl]tetraphenylbenzene; bis[(carbazolyl)phenyl]tetraphenylbenzene; [di(carbazolyl)]biphenyl; bis(tertbutylindole-d₅)benzene-d₄; di(carbazolyl-d₈)benzene-d₄; [di(carbazolyl-d₈)]biphenyl; diphenylbis{[bis(methylphenyl)amino]phenyl}biphenyldiamine; tris[(naphthylphenylamino)phenyl]amine; diphenyldi(naphthyl)biphenyldiamine; tris(phenylbenzimidazolyl)benzene; tris(phenylimidazopyridinyl)benzene; tris((methylphenyl)imidazopyridinyl)benzene; tris((biphenylyl)imidazopyridinyl)benzene; tris(methylbenzimidazolyl)triazine; tris(phenylbenzimidazolyl)triazine; aluminum(III)tris(quinolineolate); aluminum(III)bis(methylquinolineolate)(biphenylol); aluminum(III) bis(methylquinolineolate)(phenylnaphthalenol); aluminum(III)bis(methylquinolineolate)(triphenylsilanol); aluminum(III)tris[(hydroxyphenyl)phenylimidazopyridinol]; gallium(III)tris[(hydroxyphenyl)phenylimidazopyridinol]; aluminum(III)tris[(triphenylimidazolyl)phenol]; palladium(II)bis(phenylpyridyl); platinum(II)bis(phenylpyridyl); iridium(III)tris(phenylpyrazolyl); iridium(III)bis(phenylpyrazolyl)(phenylpyridyl); palladium(II)bis(phenylpyrazolyl); platinum(II)[diphenylbis(phenylpyridinyl)methane]; platinum(II){diphenylbis[(difluorophenyl)pyridinyl)methane]; palladium(II)[bis(phenylpyridinyl)propane]; palladium(II)[diphenylbis(phenylpyridinyl)methane]; platinum(II)[bis(phenylpyridinyl)methane]; platinum(II){bis[(isoquinolinyl)naphthalenyl)]methane}; platinum(II){bis[(isoquinolinyl)benzene]one}; platinum(II)[bis(phenylpyridinyl)methane]; platinum(II)[bis(bipyridinyl)one}; platinum(II)(diphenylquinoquinoline)}; platinum(II)[phenyl(phenylpyridinyl)quinoline]; platinum(II){bis[(benzothiophenyl)pyridinyl]methylene]; platinum(II){{bis[(methyl)indolyl]pyridinyl}methane]; platinum(II)[bis(phenylisoquinolinyl)methane]; platinum(II){bis[(methoxyphenyl)isoquinolinyl]methane]; platinum(II)[bis(phenacylpyridine)]platinum(II)[bis((hydroxyphenyl)pyridine)]platinum(II){bis[(hydroxybenzyli dene)]benzene}; platinum(II)[(diphenylphenanthrolinediyl)bis(phenol)]platinum(II)[[(phenylpropyl)isoquinolinediyl](phenylprop yl)isoquinoline]platinum(II)[[(phenylpropyl)isoquinolinediyl](phenylpropyl)isoquinoline]platinum(II)[(methylenebispyridinediyl)methylenebispyridine]platinum(II)[(pyridylpropyl)pyridine)diyl](phenylpropyl)benzene]platinum(II)[(isoquinolylpropyl)isoquinoline)diyl]((naphthalene)propyl)naphthalene]platinum(II)[(quinoquinolinediyl )(phenylpropyl)benzene]platinum(II)[tetraazahexacyclodotriacontapentadecene]platinum(II)[[((pyrazyl)prop yl)pyrazine)diyl](phenylpropyl)benzene]platinum(II)[bis(phenylpyridinyl)aniline]; platinum(II){[(naphthyl)pyridinyl](phenylpyridinyl)propane}; platinum(II){(phenylpyridinyl)[(benzimidazolyl)pyridinyl]propane}; platinum(II)[bis(phenylisoquinolinyl)aniline]; platinum(II)[bis(phenylbiisoquinolinyl)aniline]; platinum(II)[bis((naphthyl)pyridinyl)aniline]; platinum(II)[bis((thienyl)pyridinyl)aniline]; platinum(II)[bis(phenylpyridinyl)ethene]; platinum(II)[bis(phenylpyridinyl)one]; platinum(II)[bis((pyridyl)phenyl)aniline]; platinum(II)[bis(difluorobipyridinatoyl)propane]; platinum(II)[(difluorobipyridinatoyl){(trifluoromethylpyrazolyl)pyridinatoyl}propane]platinum(II){bis[(cyanophe nyl)pyridinyl]propane]; platinum(II)[(difluorobipyridinatoyl)(pyridinecarboxylic acidyl)propane]; platinum(II){[(phenylpyrazolyl)[(dibenzofuranyl)pyrazo]yl]propane]; platinum(II)[bis(phenylpyridinyl)propane]; platinum(II){[bis(biphenylyl)pyridinyl]propane}; iridium(III)bis[(dimethylphenyl)isobutylquinoline]acetylacetonate; iridium(III)bis[(quinolyl)isobutylbenzene]acetylacetonate; iridium(III)bis[(quinolyl)diisobutylbenzene]acetylacetonate; iridium(III)bis[(quinolyl)dimethylbenzene]acetylacetonate; iridium(III)tris[phenylisoquinoline]; iridium(III)bis[phenylisoquinoline]acetylacetonate; iridium(III)bis[(naphthyl)quinoline](tetramethyloxylatoheptenone); iridium(III)bis[(naphthyl)quinoline](phenylpyridine); iridium(III)bis(phenylpyridine) [(naphthyl)quinoline]; iridium(III)bis((thienyl)quinoline) (phenyloxylatobuteneone); iridium(III)bis[(pyridinyl)quinoline](phenylpyrazine); iridium(III)bis(biquinoline)acetylacetonate; iridium(III)bis(phenylbenzoquinoline)acetylacetonate; iridium(III)bis[(naphthyl)quinoline](phenylbenzoxazole); iridium(III)bis(phenylquinoline)acetylacetonate; iridium(III)bis[(methylphenyl)quinoline]acetylacetonate; iridium(III)bis(benzacridine)acetylacetonate; iridium(III)bis[(naphthyl)quinoline)acetylacetonate; iridium(III)bis[(pyrenyl)quinoline]acetylacetonate; iridium(III)bis[(biphenylyl)quinoline]acetylacetonate; iridium(III)bis[(phenanthrenyl)quinoline]acetylacetonate; iridium(III)bis[(methylphenyl)quinoline]acetylacetonate; iridium(III)bis[phenylquinoline]acetylacetonate; iridium(III)tris(phenylquinoline); iridium(III)bis(phenylisoquinoline)acetylacetonate; iridium(III)bis[(naphthyl)isoquinoline]acetylacetonate; iridium(III)tris{[(trifluoromethyl)phenyl]pyrazole}; iridium(III)tris[(difluorophenyl)pyridine]; iridium(III)tris[(tertbutylphenyl)pyridine]; iridium(III)tris[(thienyl)pyridine]; iridium(III)tris[(pyridyl)benzothiophene]; iridium(III)tris[(tertbutylphenyl)isoquinoline]; iridium(III)tris[(thienyl)isoquinoline]; iridium(III)tris[(dimethylfluorenyl)isoquinoline]; iridium(III)bis[(difluorophenyl)pyridine][tetra(pyrazolyl)borane]; iridium(III)bis[(difluorotrifluoromethylphenyl)pyridine](pyridinecarboxylate); iridium(III)bis(phenylpyridine)acetylacetonate; iridium(III)tris(phenylbenzoxazole); iridium(III)bis(phenylquinoline)acetylacetonate; iridium(III)bis[(pyridyl)benzothiophene]acetylacetonate; iridium(III)bis(phenylisoquinoline)acetylacetonate; iridium(III)bis[(pyridyl)naphthalene]acetylacetonate; europium(IV)tris[(thienyl)trifluorooxylatobuteneone](phenanthroline); iridium(III)tris[(naphthyl)isoquinoline]; terbium(IV)trisacetylacetonate(phenanthroline); dysprosium(IV)tris[trifluorooxylatophenylbuteneone](benzo[h]quinoline); platinum(II)[bis(phenylpyridinyl)propane]; platinum(II){bis[(difluorophenyl)pyridinyl]propane}; platinum(II){bis[(trifluoromethylpyrazolyl)pyridinyl]propane}; platinum(II){bis[(trifluoromethylpyrazolyl)pyridinyl]fluorene}; bis(diphenylethenyl)biphenyl; diphenylanthracene; di(naphthyl)anthracene; tetrakis(biphenylyl)pyrene; octakis(methylphenyl)pyrenetetraamine; dimethylquinacridone; rubrene; coumarin; {[(tetrahydrobenzoquinolizine)yl]ethenylmethylpyranylidene}propanedinitrile

### (Group 12A)

diphenyl(triphenylenyl)triazine; diphenyl(triphenylenyl)pyridine; (triphenylenyl)diphenylpyrimidine; (pyridinyl)(triphenylenyl)biphenyl; di(pyridinyl)(triphenylenyl)benzene; (pyridinyl)(pyridinyl)(triphenylenyl)benzene; di(pyridinyl)(triphenylenyl)benzene; (pyridinyl)phenyl(triphenylenyl)triazine; (pyridinyl)(triphenylenyl)pyrimidine; phenyl(triphenylenyl)pyrimidine; (pyridinyl)(triphenylenyl)benzene; phenyl(triphenylenyl)triazine; (diphenylpyridinyl)(triphenylenyl)benzene; (diphenylpyridinyl)(triphenylenyl)benzene; (diphenyltriazinyl)(triphenylenyl)benzene; di(pyridinyl)(triphenylenyl)biphenyl; di(pyridinyl)(triphenylenyl)biphenyl; (pyridinyl)(pyridinyl)(triphenylenyl)biphenyl; [di(pyridinyl)triazinyl](triphenylenyl)benzene; phenyl[(triphenylenyl)phenyl]pyrimidine; phenyl[(triphenylenyl)phenyl]triazine; (diphenylpyridinyl)(triphenylenyl)benzene; [(triphenylenyl)phenyl]diphenylpyrimidine; [(triphenylenyl)phenyl]diphenyltriazine; di(pyridinyl)(triphenylenyl)biphenyl; di(pyridinyl)(triphenylenyl)biphenyl; (pyridinyl)(pyridinyl)(triphenylenyl)biphenyl; [di(pyridinyl)triazinyl](triphenylenyl)benzene; phenyl[(triphenylenyl)phenyl]pyrimidine; phenyl[(triphenylenyl)phenyl]triazine; (triphenylenyl)(diphenylpyridinyl)biphenyl; (triphenylenyl)(diphenylpyrimidinyl)biphenyl; (triphenylenyl)(diphenyltriazinyl)biphenyl; (triphenylenyl)(pyridinyl)terbenzene; (triphenylenyl)(pyridinyl)terbenzene; (triphenylenyl)(pyridinyl)(pyridinyl)terbenzene; (triphenylenyl)[di(pyridinyl)triazinyl]biphenyl; (triphenylenyl)(phenylpyrimidinyl)biphenyl; (triphenylenyl)(phenyltriazinyl)biphenyl; (triphenylenyl)(diphenylpyridinyl)biphenyl; (triphenylenyl)(diphenylpyrimidinyl)biphenyl; (triphenylenyl)(diphenyltriazinyl)biphenyl; (triphenylenyl)di(pyridinyl)terbenzene; (triphenylenyl)di(pyridinyl)terbenzene; (triphenylenyl)(pyridinyl)(pyridinyl)terbenzene; (triphenylenyl)[di(pyridinyl)triazinyl]biphenyl; (triphenylenyl)(phenylpyrimidinyl)biphenyl; (triphenylenyl)(phenyltriazinyl)biphenyl; (diphenylpyridinyl)(triphenylenyl)biphenyl; (diphenylpyrimidinyl)(triphenylenyl)biphenyl; (diphenyltriazinyl)(triphenylenyl)biphenyl; di(pyridinyl)(triphenylenyl)terbenzene; di(pyridinyl)(triphenylenyl)terbenzene; (pyridinyl)(pyridinyl)(triphenylenyl)terbenzene; [di(pyridinyl)triazinyl](triphenylenyl)biphenyl; (phenylpyrimidinyl)(triphenylenyl)biphenyl; (phenyltriazinyl)(triphenylenyl)biphenyl; (diphenylpyridinyl)(triphenylenyl)biphenyl; (diphenylpyrimidinyl)(triphenylenyl)biphenyl; (diphenyltriazinyl)(triphenylenyl)biphenyl; di(pyridinyl)(triphenylenyl)terbenzene; di(pyridinyl)(triphenylenyl)terbenzene; (pyridinyl)(pyridinyl)(triphenylenyl)terbenzene; (di(pyridinyl)triazinyl)(triphenylenyl)biphenyl; (phenylpyrimidinyl)(triphenylenyl)biphenyl; (phenyltriazinyl)(triphenylenyl)biphenyl; (diphenylpyridinyl)(triphenylenyl)biphenyl; (diphenylpyrimidinyl)(triphenylenyl)biphenyl; (diphenyltriazinyl)(triphenylenyl)biphenyl; di(pyridinyl)(triphenylenyl)terbenzene; di(pyridinyl)(triphenylenyl)terbenzene; (pyridinyl)(pyridinyl)(triphenylenyl)terbenzene; [di(pyridinyl)triazinyl](triphenylenyl)biphenyl; (phenylpyrimidinyl)(triphenylenyl)biphenyl; (phenyltriazinyl)(triphenylenyl)biphenyl; (diphenylpyridinyl)(triphenylenyl)terbenzene; (diphenylpyrimidinyl)(triphenylenyl)terbenzene; (diphenyltriazinyl)(triphenylenyl)terbenzene; di(pyridinyl)(triphenylenyl)quaterbenzene; di(pyridinyl)(triphenylenyl)quaterbenzene; (pyridinyl)(pyridinyl)(triphenylenyl)quaterbenzene; [di(pyridinyl)triazinyl](triphenylenyl)terbenzene; (phenylpyrimidinyl)(triphenylenyl)terbenzene; (phenyltriazinyl)(triphenylenyl)terbenzene; (diphenylpyridinyl)(triphenylenyl)terbenzene; (diphenylpyrimidinyl)(triphenylenyl)terbenzene; (diphenyltriazinyl)(triphenylenyl)terbenzene; di(pyridinyl)(triphenylenyl)quaterbenzene; di(pyridinyl)(triphenylenyl)quaterbenzene; (pyridinyl)(pyridinyl)(triphenylenyl)quaterbenzene; [di(pyridinyl)triazinyl](triphenylenyl)terbenzene; (phenylpyrimidinyl)(triphenylenyl)terbenzene; (phenyltriazinyl)(triphenylenyl)terbenzene; (diphenylpyridinyl)(phenyltriphenylenyl)biphenyl; (diphenylpyrimidinyl)(triphenylenyl)terbenzene; (triphenylenyl)(diphenyltriazinyl)terbenzene; [di(pyridinyl)triazinyl](triphenylenyl)terbenzene; [(triphenylenyl)biphenylyl)terpyridine; [(triphenylenyl)biphenylyl)terpyridine; (phenyltriphenylenyl)diphenyltriazine; (methyltriphenylenyl)diphenyltriazine; (phenyltriphenylenyl)(pyridinyl)biphenyl; (phenyltriphenylenyl)di(pyridinyl)benzene; (methyltriphenylenyl)(pyridinyl)(pyridinyl)benzene; (diphenyltriphenylenyl)(pyridinyl)phenyltriazine; (pyridinyl)(phenyltriphenylenyl)pyrimidine; phenyl(phenyltriphenylenyl)pyrimidine; (diphenylpyridinyl)(methyltriphenylenyl)benzene; (diphenylpyrimidinyl)(diphenyltriphenylenyl)benzene; di(pyridinyl)(phenyltriphenylenyl)biphenyl; (pyridinyl)(pyridinyl)(methyltriphenylenyl)biphenyl; (diphenylpyridinyl)(phenyltriphenylenyl)benzene; di(pyridinyl)(triphenylenyl)biphenyl; [di(pyridinyl)triazinyl](phenyltriphenylenyl)benzene; di(pyridinyl)(phenyltriphenylenyl)terbenzene; [di(pyridinyl)triazinyl](phenyltriphenylenyl)biphenyl; (phenyltriphenylenyl)di(pyridinyl)terbenzene; [di(pyridinyl)triazinyl](dimethyltriphenylenyl)biphenyl; (diphenylpyridinyl)(phenyltriphenylenyl)biphenyl; (diphenylpyrimidinyl)(methyltriphenylenyl)biphenyl; (diphenyltriazinyl)(phenyltriphenylenyl)biphenyl; (diphenyltriazinyl)(dimethyltriphenylenyl)terbenzene; (diphenyltriazinyl)(methyltriphenylenyl)terbenzene; di(pyridinyl)(phenyltriphenylenyl)terbenzene; (diphenyltriazinyl)(phenyltriphenylenyl)biphenyl; (diphenyltriazinyl)(phenyltriphenylenyl)biphenyl; phenyl(diphenylpyridinyl)bi(carbazole)phenyl(diphenylpyridinyl)bi(carbazole)(biphenylyl)(diphenylpyridinyl)bi (carbazole)(terphenylyl)(diphenylpyridinyl)bi(carbazole); (terphenylyl)(diphenylpyridinyl)bi(carbazole); (naphthalenyl)(diphenylpyridinyl)bi(carbazole); (naphthalenyl)(diphenylpyridinyl)bi(carbazole); (triphenylenyl)(diphenylpyridinyl)bi(carbazole); (triphenylenyl)(diphenylpyridinyl)bi(carbazole); bi[(diphenylpyridinyl)carbazole]; (diphenylpyridinyl)(diphenylpyridinyl)bi(carbazole); bi[(diphenylpyridinyl)carbazole]; (dimethylfluorenyl)phenylbi(carbazole); (dimethylfluorenyl)(biphenylyl)bi(carbazole); (dimethylfluorenyl)(terphenylyl)bi(carbazole); (dibenzofuranyl)phenylbi(carbazole); (dibenzofuranyl)(biphenylyl)bi(carbazole); (dibenzofuranyl)(terphenylyl)bi(carbazole); (dibenzothiophenyl)phenylbi(carbazole); (dibenzothiophenyl)(biphenylyl)bi(carbazole); (dibenzothiophenyl)(terphenylyl)bi(carbazole); bi[phenylcarbazole]; bi[(pyridinyl)carbazole]; bi[(pyrimidinyl)carbazole]; (triphenylenyl)phenylbi(carbazole); (triphenylenyl)(pyridinyl)bi(carbazole); (triphenylenyl)phenylbi(carbazole); (triphenylenyl)(pyridinyl)bi(carbazole); phenyl(triphenylenyl)bi(carbazole); (pyridinyl)(triphenylenyl)bi(carbazole); (biphenylyl)(biphenylyl)bi(carbazole); (biphenylyl)(terphenylyl)bi(carbazole); (biphenylyl)(terphenylyl)bi(carbazole); (dimethylfluorenyl)phenylbi(carbazole); (dimethylfluorenyl)(biphenylyl)bi(carbazole); (dimethylfluorenyl)(biphenylyl)bi(carbazole); (dimethylfluorenyl)(terphenylyl)bi(carbazole); (dibenzofuranyl)(biphenylyl)bi(carbazole); (dibenzothiophenyl)(biphenylyl)bi(carbazole); (phenylcarbazolyl)phenylbi(carbazole); (dibenzofuranyl)phenylbi(carbazole); (dibenzofuranyl)(biphenylyl)bi(carbazole); (dibenzofuranyl)(terphenylyl)bi(carbazole); (dibenzofuranyl)(biphenylyl)bi(carbazole); (dibenzothiophenyl)phenylbi(carbazole); (dibenzothiophenyl)(biphenylyl)bi(carbazole); (dibenzothiophenyl)(terphenylyl)bi(carbazole); (dibenzothiophenyl)(biphenylyl)bi(carbazole); (diphenylpyridinyl)(dimethylfluorenyl)bi(carbazole); (diphenylpyridinyl)(dimethylfluorenyl)bi(carbazole); [(pyridinyl)biphenylyl](dimethylfluorenyl)bi(carbazole); [(pyridinyl)biphenylyl](dimethylfluorenyl)bi(carbazole); [(pyridinyl)biphenylyl](dimethylfluorenyl)bi(carbazole); (diphenylpyridinyl)(dibenzofuranyl)bi(carbazole); (dibenzofuranyl)[(pyridinyl)biphenylyl]bi(carbazole); (dibenzofuranyl)[(pyridinyl)biphenylyl]bi(carbazole); (dibenzofuranyl)[(pyridinyl)biphenylyl]bi(carbazole); (dibenzothiophenyl)(diphenylpyridinyl)bi(carbazole); (dibenzothiophenyl)[(pyridinyl)biphenylyl]bi(carbazole); (dibenzothiophenyl)[(pyridinyl)biphenylyl]bi(carbazole); (dibenzothiophenyl)[(pyridinyl)biphenylyl]bi(carbazole); phenyl(diphenylpyridinyl)bi(carbazole); phenyl[(pyridinyl)biphenylyl]bi(carbazole); phenyl(diphenylpyridinyl)bi(carbazole); (biphenylyl)(diphenylpyridinyl)bi(carbazole); (biphenylyl)[(pyridinyl)biphenylyl]bi(carbazole); (biphenylyl)(diphenylpyridinyl)bi(carbazole); (biphenylyl)(diphenylpyridinyl)bi(carbazole); (biphenylyl)(diphenylpyridinyl)bi(carbazole); (biphenylyl)[(pyridinyl)biphenylyl]bi(carbazole); (diphenylpyridinyl)phenylbi(carbazole); (diphenylpyridinyl)phenylbi(carbazole); [(pyridinyl)biphenylyl]phenylbi(carbazole); (diphenylpyridinyl)(biphenylyl)bi(carbazole); (diphenylpyridinyl)(biphenylyl)bi(carbazole); [(pyridinyl)biphenylyl](biphenylyl)bi(carbazole); (diphenylpyridinyl)(biphenylyl)bi(carbazole); (diphenylpyridinyl)(biphenylyl)bi(carbazole); [(pyridinyl)biphenylyl](biphenylyl)bi(carbazole); (dibenzofuranyl)(terphenylyl)bi(carbazole); (dibenzofuranyl)(biphenylyl)bi(carbazole); (dibenzofuranyl)phenylbi(carbazole); (dibenzofuranyl)(biphenylyl)bi(carbazole); (dibenzothiophenyl)(terphenylyl)bi(carbazole); (dibenzothiophenyl)(biphenylyl)bi(carbazole); (dibenzothiophenyl)phenylbi(carbazole); (dibenzothiophenyl)(biphenylyl)bi(carbazole); (biphenylyl)(diphenylpyridinyl)bi(carbazole); (diphenylpyridinyl)(biphenylyl)bi(carbazole); phenyl(diphenylpyridinyl)bi(carbazole); (diphenylpyridinyl)phenylbi(carbazole); phenyl(diphenylpyridinyl)bi(carbazole); phenyl(diphenylpyridinyl)bi(carbazole); phenyl(diphenylpyridinyl)bi(carbazole); (diphenylpyridinyl)phenylbi(carbazole); (diphenylpyridinyl)phenylbi(carbazole); (biphenylyl)(diphenylpyridinyl)bi(carbazole); phenyl(diphenylpyridinyl)bi(carbazole); bi[(biphenylyl)carbazole]; bi[(biphenylyl)carbazole]; (biphenylyl)phenylbi(carbazole)

### (Group 13A)

(carbazolyl)(triphenylenyl)biphenyl; (carbazolyl)(triphenylenyl)biphenyl; (carbazolyl)(triphenylenyl)biphenyl; (carbazolyl)(triphenylenyl)biphenyl; (carbazolyl)(triphenylenyl)terbenzene; (carbazolyl)(triphenylenyl)terbenzene; (carbazolyl)(triphenylenyl)terbenzene; (carbazolyl)(triphenylenyl)terbenzene; (carbazolyl)(triphenylenyl)quaterbenzene; (triphenylenyl)(carbazolyl)terbenzene; (phenylcarbazolyl)(triphenylenyl)biphenyl; (diphenylcarbazolyl)(triphenylenyl)biphenyl; (carbazolyl)(phenyltriphenylenyl)biphenyl; (carbazolyl)(triphenylenyl)terbenzene; (triphenylenyl)(carbazolyl)terbenzene; (carbazolyl)[(triphenylenyl)phenyl]terbenzene; (phenylcarbazolyl)(triphenylenyl)terbenzene; (diphenylcarbazolyl)(triphenylenyl)terbenzene; (carbazolyl)[(triphenylenyl)phenyl]pyridine; (carbazolyl)[(triphenylenyl)phenyl]pyridine; (carbazolyl)[(triphenylenyl)phenyl]pyridine; (carbazolyl)[(triphenylenyl)phenyl]pyridine; [(carbazolyl)pyridinyl](triphenylenyl)biphenyl; [(carbazolyl)pyridinyl](triphenylenyl)biphenyl; [(carbazolyl)pyridinyl](triphenylenyl)biphenyl; [(carbazolyl)pyridinyl](triphenylenyl)biphenyl; [(carbazolyl)pyridinyl](triphenylenyl)terbenzene; (carbazolyl)phenyl[(triphenylenyl)phenyl]pyridine; (phenylcarbazolyl)[(triphenylenyl)phenyl]pyridine; (diphenylcarbazolyl)[(triphenylenyl)phenyl]pyridine; (carbazolyl)[(phenyltriphenylenyl)phenyl]pyridine; [(carbazolyl)pyridinyl](triphenylenyl)biphenyl; (carbazolyl)phenyl[(triphenylenyl)phenyl]pyridine; (triphenylenyl)[(carbazolyl)pyridinyl]terbenzene; [(phenylcarbazolyl)pyridinyl](triphenylenyl)biphenyl; [(diphenylcarbazolyl)pyridinyl](triphenylenyl)biphenyl; [(carbazolyl)phenyl][(triphenylenyl)phenyl]pyridine; [(carbazolyl)phenyl][(triphenylenyl)biphenylyl]pyridine; (carbazolyl)[(triphenylenyl)phenyl]pyrimidine; (carbazolyl)[(triphenylenyl)phenyl]pyrimidine; (carbazolyl)[(triphenylenyl)phenyl]pyrimidine; (carbazolyl)[(triphenylenyl)phenyl]pyridine; [(triphenylenyl)phenyl](carbazolyl)pyridine; [(triphenylenyl)phenyl](carbazolyl)pyridine; (carbazolyl)[(triphenylenyl)phenyl]pyridine; [(carbazolyl)pyridinyl](triphenylenyl)biphenyl; (carbazolyl)(phenyltriphenylenyl)biphenyl; (carbazolyl)(diphenyltriphenylenyl)biphenyl; [(carbazolyl)biphenylyl][(carbazolyl)phenyl]triphenylene; bis[(carbazolyl)biphenylyl]triphenylene; bis[(carbazolyl)biphenylyl]triphenylene; [(carbazolyl)biphenylyl][(carbazolyl)phenyl]triphenylene; (carbazolyl)[(triphenylenyl)phenyl]pyridine; (diphenyltriazinyl)(biphenylyl)indolocarbazole; (diphenyltriazinyl)(terphenylyl)indolocarbazole; [(biphenylyl)phenyltriazinyl](biphenylyl)indolocarbazole; [di(biphenylyl)triazinyl]phenylindolocarbazole; [(biphenylyl)phenyltriazinyl](biphenylyl)indolocarbazole; [di(biphenylyl)triazinyl](biphenylyl)indolocarbazole; [(biphenylyl)phenyltriazinyl](terphenylyl)indolocarbazole; [di(biphenylyl)triazinyl](terphenylyl)indolocarbazole; [(biphenylyl)phenyltriazinyl]thiaazaindenofluorene; [di(biphenylyl)triazinyl]thiaazaindenofluorene; [bis(dimethylfluorenyl)triazinyl]phenylindolocarbazole; [(biphenylyl)phenyltriazinyl]oxazaindenofluorene; [di(biphenylyl)triazinyl]oxazaindenofluorene; [bis(dimethylfluorenyl)triazinyl]thiaazaindenofluorene; (diphenyltriazinyl)phenylindolocarbazole; (diphenyltriazinyl)(biphenylyl)indolocarbazole; [phenyl(biphenylyl)triazinyl]phenylindolocarbazole; [di(biphenylyl)triazinyl]phenylindolocarbazole; [phenyl(biphenylyl)triazinyl]thiaazaindenofluorene; [di(biphenylyl)triazinyl]thiaazaindenofluorene; [phenyl(biphenylyl)triazinyl]azaoxaindenofluorene; [di(biphenylyl)triazinyl]azaoxaindenofluorene; [bis(dimethylfluorenyl)triazinyl]thiaazaindenofluorene; [bis(dimethylfluorenyl)triazinyl]azaoxaindenofluorene; [bis(dimethylfluorenyl)triazinyl]phenylindolocarbazole; (diphenyltriazinyl)(biphenylyl)indolocarbazole; (diphenyltriazinyl)(terphenylyl)indolocarbazole; [(biphenylyl)phenyltriazinyl](biphenylyl)indolocarbazole; [di(biphenylyl)triazinyl]phenylindolocarbazole; [di(biphenylyl)triazinyl](biphenylyl)indolocarbazole; [di(dimethylfluorenyl)triazinyl]phenylindolocarbazole; [(biphenylyl)phenyltriazinyl]thiaazaindenofluorene; [di(biphenylyl)triazinyl]thiaazaindenofluorene; [bis(dimethylfluorenyl)triazinyl]thiaazaindenofluorene; [(biphenylyl)phenyltriazinyl]oxaindenofluorene; [di(biphenylyl)triazinyl]azaoxaindenofluorene; [bis(dimethylfluorenyl)triazinyl]azaoxaindenofluorene; [(biphenylyl)phenyltriazinyl]dimethylindenocarbazole; [di(biphenylyl)triazinyl]dimethylindenocarbazole; [bis(dimethylfluorenyl)triazinyl]dimethylindenocarbazole; (diphenyltriazinyl)(biphenylyl)indolocarbazole; (diphenyltriazinyl)(terphenylyl)indolocarbazole; [(biphenylyl)phenyltriazinyl](biphenylyl)indolocarbazole; [bis(dimethylfluorenyl)triazinyl]phenylindolocarbazole; [bis(dimethylfluorenyl)triazinyl](biphenylyl)indolocarbazole; [(biphenylyl)phenyltriazinyl]azathiaindenofluorene; [di(biphenylyl)triazinyl]azathiaindenofluorene; [bis(dimethylfluorenyl)triazinyl]azathiaindenofluorene; [(biphenylyl)phenyltriazinyl]azaoxaindenofluorene; [di(biphenylyl)triazinyl]azaoxaindenofluorene; [bis(dimethylfluorenyl)triazinyl]azaoxaindenofluorene; (biphenylyl)[(diphenyltriazinyl)phenyl]indolocarbazole; (terphenylyl)[(diphenyltriazinyl)phenyl]indolocarbazole; phenyl{[phenyl(biphenylyl)triazinyl]phenyl}indolocarbazole; phenyl{[di(biphenylyl)triazinyl]phenyl}indolocarbazole; phenyl{[bis(dimethylfluorenyl)triazinyl]phenyl}indolocarbazole; (biphenylyl){[(biphenylyl)phenyltriazinyl]phenyl}indolocarbazole; (biphenylyl){[di(biphenylyl)triazinyl]phenyl}indolocarbazole; (terphenylyl){[(biphenylyl)phenyltriazinyl]phenyl}indolocarbazole; [(diphenyltriazinyl)phenyl]thiaazaindenofluorene; {[phenyl(biphenylyl)triazinyl]phenyl}thiaazaindenofluorene; {[di(biphenylyl)triazinyl]phenyl}thiaazaindenofluorene; {[bis(dimethylfluorenyl)triazinyl]phenyl}thiaazaindenofluorene; [(diphenyltriazinyl)phenyl]oxazaindenofluorene; {[phenyl(biphenylyl)triazinyl]phenyl}oxazaindenofluorene; {[di(biphenylyl)triazinyl]phenyl}oxazaindenofluorene; {[bis(dimethylfluorenyl)triazinyl]phenyl}oxazaindenofluorene; [(phenylpyridinyl)phenyl](diphenyltriazinyl)indolocarbazole; (terphenylyl)(diphenyltriazinyl)indolocarbazole; (terphenylyl)[(biphenylyl)phenyltriazinyl]indolocarbazole; phenyl(diphenyltriazinyl)dimethyldiazadiindenofluorene; (biphenylyl)(diphenyltriazinyl)dimethyldiazaindenofluorene; (biphenylyl)[(biphenylyl)phenyltriazinyl]dimethyldiazaindenofluorene; tetramethyl[(biphenylyl)phenyltriazinyl]azadiindenofluorene; tetramethyl[di(biphenylyl)triazinyl]azadiindenofluorene; [(biphenylyl)phenyltriazinyl]azadithiadiindenofluorene; [di(biphenylyl)triazinyl]azadithiadiindenofluorene; [(biphenylyl)phenyltriazinyl]azadithiadiindenofluorene; [(biphenylyl)phenyltriazinyl]azadioxydiindenofluorene; [di(biphenylyl)triazinyl]azadithiadiindenofluorene; [di(biphenylyl)triazinyl]azadioxydiindenofluorene

### (Group 14A)

(biphenylyl)(diphenyltriazinyl)carbazole; iridium(III)bis[(dimethylphenyl)isobutylquinoline](acetylacetonate); phenyl(diphenyltriazinyl)indolocarbazole; iridium(III)bis(phenylmethyl-d₃pyridine)(diphenylmethyl-d₃pyridine); (biphenylyl)(diphenyltriazinyl)carbazole; (terphenylyl)(diphenyltriazinyl)carbazole; triphenyl(diphenyltriazinyl)carbazole; (biphenylyl)[phenyl(biphenylyl)triazinyl]carbazole; diphenyl[phenyl(biphenylyl)triazinyl]carbazole; (biphenylyl)[(diphenyltriazinyl)phenyl]carbazole; phenyl[di(biphenylyl)triazinyl]carbazole; diphenyl[(diphenyltriazinyl)phenyl]carbazole; diphenyl[di(biphenylyl)triazinyl]carbazole; phenyl{[di(biphenylyl)triazinyl]phenyl}carbazole; di(biphenylyl)(diphenyltriazinyl)carbazole; (dimethylfluorenyl)(diphenyltriazinyl)carbazole; [(dimethylfluorenyl)phenyl](diphenyltriazinyl) carbazole; (dimethylfluorenyl)[phenyl(biphenylyl)triazinyl]carbazole; [(dimethylfluorenyl)phenyl][phenyl(biphenylyl)triazinyl]carbazole; (dimethylfluorenyl)[di(biphenylyl)triazinyl]carbazole; (phenylcarbazolyl)(diphenyltriazinyl)carbazole; (triphenylenyl)[phenyl(biphenylyl)triazinyl]carbazole; (phenylcarbazolyl)[phenyl(biphenylyl)triazinyl]carbazole; phenyl[phenyl(dimethylfluorenyl)triazinyl]carbazole; (triphenylenyl)(diphenyltriazinyl)carbazole; (biphenylyl)[phenyl(dimethylfluorenyl)triazinyl]carbazole; (terphenylyl)[phenyl(dimethylfluorenyl)triazinyl]carbazole; (quaterphenylyl)[phenyl(biphenylyl)triazinyl]carbazole; phenyl[(biphenylyl)(dimethylfluorenyl)triazinyl]carbazole; [(terphenylyl)phenyl](diphenyltriazinyl)carbazole; phenyl[bis(dimethylfluorenyl)triazinyl]carbazole; [(terphenylyl)phenyl][phenyl(biphenylyl)triazinyl]carbazole; (terphenylyl)(diphenyltriazinyl)carbazole; (biphenylyl)(diphenyltriazinyl)carbazole; (biphenylyl)[phenyl(biphenylyl)triazinyl]carbazole; (biphenylyl)[di(biphenylyl)triazinyl]carbazole; (terphenylyl)(diphenyltriazinyl)carbazole; (terphenylyl)(diphenyltriazinyl)carbazole; (biphenylyl)(diphenyltriazinyl)carbazole; (terphenylyl)[phenyl(biphenylyl)triazinyl]carbazole; (biphenylyl)[phenyl(biphenylyl)triazinyl]carbazole; (terphenylyl)[di(biphenylyl)triazinyl]carbazole; (terphenylyl)(diphenyltriazinyl)carbazole; (terphenylyl)[phenyl(biphenylyl)triazinyl]carbazole; (terphenylyl)[phenyl(biphenylyl)triazinyl]carbazole; phenyl[(biphenylyl)(biphenylyl)triazinyl]carbazole; phenyl[(biphenylyl)phenyltriazinyl]carbazole; (biphenylyl)[phenyl(biphenylyl)triazinyl]carbazole; (biphenylyl)[(biphenylyl)(biphenylyl)triazinyl]carbazole; phenyl[di(biphenylyl)triazinyl]carbazole; (biphenylyl) [phenyl(dimethylfluorenyl)triazinyl]carbazole; (dimethylfluorenyl)[(biphenylyl)phenyltriazinyl]carbazole; (biphenylyl){phenyl[(dimethylfluorenyl)phenyl]triazinyl}carbazole; [(dimethylfluorenyl)phenyl][phenyl(biphenylyl)triazinyl]carbazole; iridium(III)bis[(dimethylphenyl)quinoline](acetylacetonate); iridium(III)bis[(dimethylphenyl)quinoline](diethylnonanedionate); iridium(III)bis[(dimethylphenyl)quinoline](dimethylheptanedionate); iridium(III)bis[(dimethylphenyl)isobutylquinoline](dimethylheptanedionate); iridium(III)bis[(dimethylphenyl)quinoline](dimethylnonanedionate); iridium(III)bis[(dimethylphenyl)isobutylquinoline](dimethylnonanedionate); iridium(III)bis[(dimethylphenyl)isobutylquinoline](acetylacetonate); iridium(III)bis[(dimethylphenyl)isobutylquinoline](acetylacetonate); iridium(III)bis[(dimethylphenyl)isobutylquinoline](diethylnonanedionate); iridium(III)bis[(dimethylphenyl)isobutylquinoline](diethylnonanedionate); iridium(III)bis[(dimethylphenyl)isobutylquinoline](dimethylheptanedionate); iridium(III)bis[(dimethylphenyl)isobutylquinoline](diethylnonanedionate); iridium(III)bis[(dimethylphenyl)isobutylquinoline](dimethylnonanedionate); iridium(III)bis[(dimethylphenyl)isobutylquinoline](dimethylheptanedionate); iridium(III)bis[(dimethylphenyl)isobutylquinoline](acetylacetonate); iridium(III)bis[(dimethylphenyl)isobutylquinoline](dimethylnonanedionate); iridium(III)bis[(dimethylphenyl)cyclopentylquinoline](acetylacetonate); iridium(III)bis[(dimethylphenyl)cyclopentylquinoline](acetylacetonate); iridium(III)bis[(dimethylphenyl)cyclopentylquinoline](diethylnonanedionate); iridium(III)bis[(dimethylphenyl)cyclopentylquinoline](diethylnonanedionate); iridium(III)bis[(dimethylphenyl)cyclopentylquinoline](dimethylheptanedionate); iridium(III)bis[(dimethylphenyl)cyclopentylquinoline](dimethylheptanedionate); iridium(III)bis[(dimethylphenyl)cyclopentylquinoline](dimethylnonanedionate); iridium(III)bis[(dimethylphenyl)cyclopentylquinoline](dimethylnonanedionate); iridium(III)bis[(dimethylphenyl)cyclopentylquinoline](acetylacetonate); iridium(III)bis[(dimethylphenyl)cyclopentylquinoline](dimethylnonanedionate); iridium(III)bis[(dimethylphenyl)cyclopentylquinoline](diethylnonanedionate); iridium(III)bis[(dimethylphenyl)isopropylquinoline](acetylacetonate); iridium(III)bis[(dimethylphenyl)isopropylquinoline](diethylnonanedionate); iridium(III)bis[(dimethylphenyl)cyclopentylquinoline](dimethylheptanedionate); iridium(III)bis[(dimethylphenyl)isopropylquinoline](dimethylheptanedionate); iridium(III)bis[(dimethylphenyl)isopropylquinoline](dimethylnonanedionate); iridium(III)bis[(dimethylphenyl)isopropylquinoline](dimethylnonanedionate); iridium(III)bis[(dimethylphenyl)isopropylquinoline](acetylacetonate); iridium(III)bis[(dimethylphenyl)isopropylquinoline](acetylacetonate); iridium(III)bis[(dimethylphenyl)isopropylquinoline](diethylnonanedionate); iridium(III)bis[(dimethylphenyl)isopropylquinoline](diethylnonanedionate); iridium(III)bis[(dimethylphenyl)isopropylquinoline](diethylpentanedionate); iridium(III)bis[(dimethylphenyl)isopropylquinoline](diethylpentanedionate); iridium(III)bis[(dimethylphenyl)isopropylquinoline](diethylnonanedionate); iridium(III)bis(phenylpyridine)(diphenylpyridine); iridium(III)bis(phenylpyridine)[phenyl(methylphenyl)pyridine); iridium(III)bis(phenylpyridine)[phenyl(ethylphenyl)pyridine]; iridium(III)bis(phenylpyridine)[phenyl(isopropylphenyl)pyridine]; iridium(III)bis(phenylpyridine)[phenyl(ethylfluorophenyl)pyridine]; iridium(III)bis(phenylpyridine)[phenyl(fluorophenyl)pyridine]; iridium(III)bis(phenylpyridine)[phenyl(isopropylfluorophenyl)pyridine]; iridium(III)bis(phenylpyridine)[phenyl(methylfluorophenyl)pyridine]; iridium(III)bis(phenylpyridine)[phenyl(isobutylfluorophenyl)pyridine]; iridium(III)bis(phenylpyridine)(diphenylpyridine); iridium(III)bis(phenylpyridine)[phenyl(isobutylphenyl)pyridine]; iridium(III)bis(phenylpyridine)[phenyl(methylphenyl)pyridine]; iridium(III)bis(phenylpyridine)[phenyl(ethylphenyl)pyridine]; iridium(III)bis(phenylpyridine)[phenyl(isopropylphenyl)pyridine]; iridium(III)bis(phenylpyridine)[phenyl(isobutylphenyl)pyridine]; iridium(III)bis(phenylpyridine)[phenyl(isopropylfluorophenyl)pyridine]iridium(III)bis(phenylpyridine)[phenyl(flu orophenyl)pyridine]; iridium(III)bis(phenylpyridine)[phenyl(isobutylfluorophenyl)pyridine]iridium(III)bis(phenylpyridine)[phenyl(met hylfluorophenyl)pyridine]; iridium(III)bis(phenylpyridine)[diphenylpyridine]iridium(III)bis(phenylpyridine)[phenyl(ethylfluorophenyl)pyridi ne]; iridium(III)bis(phenylmethylpyridine)[phenyl(methylphenyl)pyridine]; iridium(III)bis(phenylmethylpyridine)[phenyl(ethylphenyl)pyridine]; iridium(III)bis(phenylmethylpyridine)[phenyl(methylfluorophenyl)pyridine]; iridium(III)bis(phenylmethylpyridine)[phenyl(isopropylphenyl)pyridine]; iridium(III)bis(phenylmethylpyridine)[phenyl(ethylfluorophenyl)pyridine]; iridium(III)bis(phenylmethylpyridine)[phenyl(fluorophenyl)pyridine]; iridium(III)bis(phenylmethylpyridine)[phenyl(isopropylfluorophenyl)pyridine]; iridium(III)bis(phenylmethylpyridine)[phenyl(isobutylfluorophenyl)pyridine]; iridium(III)bis(phenylmethylpyridine)[phenyl(isopropylphenyl)pyridine]; iridium(III)bis(phenylmethylpyridine)[diphenylpyridine]; iridium(III)bis(phenylmethylpyridine)[phenyl(isobutylphenyl)pyridine]; iridium(III)bis(phenylmethylpyridine)[phenyl(methylphenyl)pyridine]; iridium(III)bis(phenylmethylpyridine)[phenyl(ethylphenyl)pyridine]; iridium(III)bis(phenylmethylpyridine)[phenyl(isopropylphenyl)pyridine]; iridium(III)bis(phenylmethylpyridine)[phenyl(ethylfluorophenyl)pyridine]; iridium(III)bis(phenylmethylpyridine)[phenyl(isobutylphenyl)pyridine]; iridium(III)bis(phenylmethylpyridine)[phenyl(isopropylfluorophenyl)pyridine]; iridium(III)bis(phenylmethylpyridine)[phenyl(fluorophenyl)pyridine]; iridium(III)bis(phenylmethylpyridine)[phenyl(isobutylfluorophenyl)pyridine]; iridium(III)bis(phenylmethylpyridine)[phenyl(methylfluorophenyl)pyridine]; iridium(III)bis(phenylpyridine)[phenyl((methyl-d₃)phenyl)pyridine]; iridium(III)bis(phenylpyridine)[phenyl((ethyl-d₅)phenyl)pyridine]; iridium(III)bis(phenylpyridine) [phenyl((ethyl-d₅)fluorophenyl)pyridine]; iridium(III)bis(phenylpyridine)[phenyl((isopropyl-d₇)phenyl)pyridine]; iridium(III)bis(phenylpyridine)[phenyl((isopropyl-d₇)fluorophenyl)pyridine]; iridium(III)bis(phenylpyridine)[phenyl((methyl-d₃)fluorophenyl)pyridine]; iridium(III)bis(phenylpyridine)[phenyl((isobutyl-d₉)fluorophenyl)pyridine]; iridium(III)bis(phenylpyridine)[phenyl(dimethyl-d₆)phenyl)pyridine]; iridium(III)bis(phenylpyridine)[phenyl((isobutyl-d₉)phenyl)pyridine]; iridium(III)bis(phenylpyridine)[phenyl((methyl-d₃)phenyl)pyridine]; iridium(III)bis(phenylpyridine)[phenyl((ethyl-d₆)phenyl)pyridine]; iridium(III)bis(phenylpyridine)[phenyl((isopropyl-d₇)phenyl)pyridine]; iridium(III)bis(phenylpyridine)[phenyl((isobutyl-d₉)phenyl)pyridine]; iridium(III)bis(phenylpyridine)[phenyl((isobutyl-d₉)fluorophenyl)pyridine]; iridium(III)bis(phenylpyridine)[phenyl((methyl-d₃)fluorophenyl)pyridine]; iridium(III)bis(phenyl(methyl-d₃)pyridine)[diphenylpyridine]; iridium(III)bis(phenylpyridine)[phenyl((ethyl-d₅)fluorophenyl)pyridine]; iridium(III)bis(phenyl(methyl-d₃)pyridine)[phenyl((methyl-d₃)phenyl)pyridine]; iridium(III)bis(phenylpyridine)[phenyl((isopropyl-d₇)fluorophenyl)pyridine]; iridium(III)bis(phenyl(methyl-d₃)pyridine)[phenyl((ethyl-d₅)phenyl)pyridine]; iridium(III)bis(phenyl(methyl-d₃)pyridine)[phenyl((isopropyl-d₇)phenyl)pyridine]; iridium(III)bis(phenyl(methyl-d₃)pyridine)[phenyl((isopropyl-d₇)fluorophenyl)pyridine]; iridium(III)bis(phenyl(methyl-d₃)pyridine)[phenyl((methyl-d₃)fluorophenyl)pyridine]; iridium(III)bis(phenyl(methyl-d₃)pyridine)[phenyl((isobutyl-d₉)fluorophenyl)pyridine]; iridium(III)bis(phenyl(methyl-d₃)pyridine)[phenyl((ethyl-d₅)fluorophenyl)pyridine]; iridium(III)bis(phenyl(methyl-d₃)pyridine)[phenyl(fluorophenyl)pyridine]; iridium(III)bis(phenyl(methyl-d₃)pyridine)[diphenylpyridine]; iridium(III)bis(phenyl(methyl-d₃)pyridine)[phenyl((isobutyl-d₉)phenyl)pyridine]; iridium(III)bis(phenyl(methyl-d₃)pyridine)[phenyl((methyl-d₃)phenyl)pyridine]; iridium(III)bis(phenyl(methyl-d₃)pyridine)[phenyl((ethyl-d₅)phenyl)pyridine]; iridium(III)bis(phenyl(methyl-d₃)pyridine)[phenyl((isopropyl-d₇)phenyl)pyridine]; iridium(III)bis(phenyl(methyl-d₃)pyridine)[phenyl((isobutyl-d₉)phenyl)pyridine]; iridium(III)bis(phenyl(methyl-d₃)pyridine)[phenyl((isopropyl-d₇)fluorophenyl)pyridine]; iridium(III)bis(phenyl(methyl-d₃)pyridine)[phenyl(fluorophenyl)pyridine]; iridium(III)bis(phenyl(methyl-d₃)pyridine)[phenyl((isobutyl-d₉)fluorophenyl)pyridine]; iridium(III)bis(phenyl(methyl-d₃)pyridine)[phenyl((methyl-d₃)fluorophenyl)pyridine]; iridium(III)bis(phenylpyridine)[phenylbipyridine]; iridium(III)bis(phenyl(methyl-d₃)pyridine)[phenyl((ethyl-d₅)fluorophenyl)pyridine]; iridium(III)bis(phenylpyridine)[phenylbipyridine]; iridium(III)bis(phenylpyridine)[phenylbipyridine]; iridium(III)bis(phenylpyridine)[isobutylphenylbipyridine]; iridium(III)bis(phenylpyridine)[methylphenylbipyridine]; iridium(III)bis(phenylpyridine)[(phenylpyridinyl)pyrimidine]; iridium(III)bis(phenylpyridine)[ethylphenylbipyridine]; iridium(III)bis(phenylpyridine)[(phenylpyridinyl)pyrazine]; iridium(III)bis(phenylpyridine)[isopropylphenylbipyridine]; iridium(III)bis(phenylpyridine)[(phenylpyridinyl)pyrimidine]; iridium(III)bis(phenylpyridine)[(phenylpyridinyl)pyrimidine]; iridium(III)bis(phenylpyridine)[(phenylpyridinyl)dimethyltriazine]; iridium(III)bis(phenylpyridine)[(phenylpyridinyl)triazine]; iridium(III)bis(phenylpyridine)[phenylbipyridine]; iridium(III)bis(phenylpyridine)[methyl(phenylpyridinyl)pyrimidine]; iridium(III)bis(phenylpyridine)[phenylbipyridine]; iridium(III)bis(phenylpyridine)[methyl(phenylpyridinyl)pyrimidine]; iridium(III)bis(phenylpyridine)[phenylbipyridine]; iridium(III)bis(phenylpyridine)[methylphenylbipyridine]; iridium(III)bis(phenylpyridine)[(phenylpyridinyl)pyrimidine]; iridium(III)bis(phenylpyridine)[ethylphenylbipyridine]; iridium(III)bis(phenylpyridine)[(phenylpyridinyl)pyrazine]; iridium(III)bis(phenylpyridine)[isopropylphenylbipyridine]; iridium(III)bis(phenylpyridine)[(phenylpyridinyl)pyrimidine]; iridium(III)bis(phenylpyridine)[isobutylphenylbipyridine]; iridium(III)bis(phenylpyridine)[(phenylpyridinyl)pyrimidine]; iridium(III)bis(phenylpyridine)[(phenylpyridinyl)triazine]; iridium(III)bis(phenylpyridine)[(methyl-d₃)phenylbipyridine]; iridium(III)bis(phenylpyridine)[methyl(phenylpyridinyl)pyrimidine]; iridium(III)bis(phenylpyridine)[(ethyl-d₅)phenylbipyridine]; iridium(III)bis(phenylpyridine)[methyl(phenylpyridinyl)pyrimidine]; iridium(III)bis(phenylpyridine)[dimethyl(phenylpyridinyl)triazine]; iridium(III)bis(phenylpyridine) [(isopropyl-d₇)phenylbipyridine]; iridium(III)bis(phenylpyridine)[(isobutyl-d₉)phenylbipyridine]; iridium(III)bis(phenylpyridine)[(dimethyl-d₆)(phenylpyridinyl)triazine]; iridium(III)bis(phenylpyridine)[(methyl-d₃)(phenylpyridinyl)pyrimidine]; iridium(III)bis(phenylpyridine)[(methyl-d₃)phenylbipyridine]; iridium(III)bis(phenylpyridine)[(ethyl-d₅)phenylbipyridine]; iridium(III)bis(phenylpyridine) [(methyl-d₃)(phenylpyridinyl)pyrimidine]; iridium(III)bis(phenylpyridine)[(isopropyl-d₇)phenylbipyridine]; iridium(III)bis(phenylpyridine)[(isobutyl-d₉)phenylbipyridine]; iridium(III)bis(phenylmethylpyridine)(phenylbipyridine); iridium(III)bis(phenylpyridine)[(methyl-d₃)(phenylpyridinyl)pyrimidine]; iridium(III)bis(phenylmethylpyridine)(phenylbipyridine); iridium(III)bis(phenylpyridine)[(methyl-d₃)(phenylpyridinyl)pyrimidine]; iridium(III)bis(phenylmethylpyridine)(phenylbipyridine); iridium(III)bis(phenylpyridine)[(dimethyl-d₆)(phenylpyridinyl)triazine]; iridium(III)bis(phenylmethylpyridine)(methylphenylbipyridine); iridium(III)bis(phenylmethylpyridine)(ethylphenylbipyridine); iridium(III)bis(phenylmethylpyridine)[(phenylpyridinyl)pyrimidine]; iridium(III)bis(phenylmethylpyridine)(isopropylphenylbipyridine); iridium(III)bis(phenylmethylpyridine)[(phenylpyridinyl)pyrazine]; iridium(III)bis(phenylmethylpyridine)[(phenylpyridinyl)pyrimidine]; iridium(III)bis(phenylmethylpyridine)(isobutylphenylbipyridine); iridium(III)bis(phenylmethylpyridine)[(phenylpyridinyl)pyrimidine]; iridium(III)bis(phenylmethylpyridine)[(phenylpyridinyl)triazine]; iridium(III)bis(phenylmethylpyridine)(phenylbipyridine); iridium(III)bis(phenylmethylpyridine)[methyl(phenylpyridinyl)pyrimidine]; iridium(III)bis(phenylmethylpyridine)(phenylbipyridine); iridium(III)bis(phenylmethylpyridine)[methyl(phenylpyridinyl)pyrimidine]; iridium(III)bis(phenylmethylpyridine)(phenylbipyridine); iridium(III)bis(phenylpyridine)[dimethyl(phenylpyridinyl)triazine]; iridium(III)bis(phenylmethylpyridine)(methylphenylbipyridine); iridium(III)bis(phenylmethylpyridine)(ethylphenylbipyridine); iridium(III)bis(phenylmethylpyridine)[(phenylpyridinyl)pyrazine]; iridium(III)bis(phenylmethylpyridine)(isopropylphenylbipyridine); iridium(III)bis(phenylmethylpyridine)[(phenylpyridinyl)pyrimidine]; iridium(III)bis(phenylmethylpyridine)(isobutylphenylbipyridine); iridium(III)bis(phenylmethylpyridine)[(phenylpyridinyl)pyrimidine]; iridium(III)bis(phenylmethylpyridine)[(phenylpyridinyl)pyrimidine]; iridium(III)bis(phenylmethylpyridine)[(phenylpyridinyl)triazine]; iridium(III)bis(phenylmethylpyridine)[methyl(phenylpyridinyl)pyrimidine]; iridium(III)bis(phenylmethylpyridine)[(methyl-d₃)phenylbipyridine]; iridium(III)bis(phenylmethylpyridine)[methyl(phenylpyridinyl)pyrimidine]; iridium(III)bis(phenyl(methyl-d₃)pyridine)[(ethyl-d₆)phenylbipyridine]; iridium(III)bis(phenylmethylpyridine)[dimethyl(phenylpyridinyl)triazine]; iridium(III)bis[phenyl(methyl-d₃)pyridine][phenylbipyridine]; iridium(III)bis[phenyl(methyl-d₃)pyridine][(isopropyl-d₇)phenylbipyridine]; iridium(III)bis[phenyl(methyl-d₃)pyridine][(isobutyl-d₉)phenylbipyridine]; iridium(III)bis(phenyl(methyl-d₃)pyridine)[(phenylpyridinyl)pyrimidine]; iridium(III)bis(phenyl(methyl-d₃)pyridine)[(phenylpyridinyl)pyrimidine]; iridium(III)bis(phenyl(methyl-d₃)pyridine)[(phenylpyridinyl)triazine]; iridium(III)bis(phenyl(methyl-d₃)pyridine)[(phenylpyridinyl)pyrazine]; iridium(III)bis(phenyl(methyl-d₃)pyridine)[(methyl-d₃)(phenylpyridinyl)pyrimidine]; iridium(III)bis(phenyl(methyl-d₃)pyridine)[(phenylpyridinyl)pyrimidine]; iridium(III)bis(phenyl(methyl-d₃)pyridine)[(methyl-d₃)(phenylpyridinyl)pyrimidine]; iridium(III)bis(phenyl(methyl-d₃)pyridine)[(dimethyl-d₆)(phenylpyridinyl)triazine]; iridium(III)bis[phenyl(methyl-d₃)pyridine][(isopropyl-d₉)phenylbipyridine]; iridium(III)bis[phenyl(methyl-d₃)pyridine][phenylbipyridine]; iridium(III)bis[phenyl(methyl-d₃)pyridine][(isobutyl-d₉)phenylbipyridine]; iridium(III)bis[phenyl(methyl-d₃)pyridine][(methyl-d₃)phenylbipyridine]; iridium(III)bis(phenyl(methyl-d₃)pyridine)[(phenylpyridinyl)pyrimidine]; iridium(III)bis[phenyl(methyl-d₃)pyridine][(ethyl-d₇)phenylbipyridine]; iridium(III)bis(phenyl(methyl-d₃)pyridine)[(phenylpyridinyl)pyrazine]; iridium(III)bis(phenyl(methyl-d₃)pyridine)[(phenylpyridinyl)pyrimidine]; iridium(III)bis(phenyl(methyl-d₃)pyridine)[(phenylpyridinyl)pyrimidine]; iridium(III)bis(phenyl(methyl-d₃)pyridine)[(phenylpyridinyl)triazine]; iridium(III)bis(phenyl(methyl-d₃)pyridine)[(methyl-d₃)(phenylpyridinyl)pyrimidine]; iridium(III)bis(phenyl(methyl-d₃)pyridine)[(methyl-d₃)(phenylpyridinyl)pyrimidine]; iridium(III)bis(phenyl(methyl-d₃)pyridine)[(dimethyl-d₆)(phenylpyridinyl)triazine]; (biphenylyl)(diphenyltriazinyl)indolocarbazole; phenyl[(biphenylyl)phenyltriazinyl]indolocarbazole; [(biphenylyl)phenyltriazinyl]oxazaindenofluorene; [(biphenylyl)phenyltriazinyl]thiaazaindenofluorene; [(dimethylfluorenyl)phenyltriazinyl]thiaazaindenofluorene; [diphenyltriazinyl]thiaazaindenofluorene; phenyl[(dimethylfluorenyl)phenyltriazinyl]indolocarbazole; phenyl(dimethyltriazinyl)indolocarbazole; phenyl[phenyl(terphenylyl)triazinyl]indolocarbazole; (diphenyltriazinyl)azaoxaindenofluorene; [(biphenylyl)phenyltriazinyl)azaoxaindenofluorene; (diphenyltriazinyl)thiaazaindenofluorene; [(dimethylfluorenyl)phenyltriazinyl]thiaazaindenofluorene; [(biphenylyl)phenyltriazinyl]thiaazaindenofluorene; [(dimethylfluorenyl)phenyltriazinyl]azaoxaindenofluorene; phenyl[(dimethylfluorenyl)phenyltriazinyl]indolocarbazole; phenyl[(biphenylyl)phenyltriazinyl]indolocarbazole; phenyl(diphenyltriazinyl)indolocarbazole; phenyl[(dimethylfluorenyl)phenyltriazinyl]indolocarbazole; (biphenylyl)(diphenyltriazinyl)indolocarbazole; (diphenyltriazinyl)thiaazaindenofluorene; [(biphenylyl)phenyltriazinyl]thiaazaindenofluorene; [(biphenylyl)phenyltriazinyl]azaoxaindenofluorene; [(dimethylfluorenyl)phenyltriazinyl]thiaazaindenofluorene; [(dimethylfluorenyl)phenyltriazinyl]azaoxaindenofluorene; (diphenyltriazinyl)azaoxaindenofluorene; (diphenyltriazinyl)dimethylazaindenofluorene; [(biphenylyl)phenyltriazinyl]dimethylazaindenofluorene; (diphenyltriazinyl)phenylindolocarbazole; [(dimethylfluorenyl)phenyltriazinyl]dimethylazaindenofluorene; [(biphenylyl)phenyltriazinyl]phenylindolocarbazole; (diphenyltriazinyl)phenylindolocarbazole; (dimethylfluorenyl)phenylindolocarbazole; (diphenyltriazinyl)azathiaindenofluorene; (diphenyltriazinyl)azaoxaindenofluorene; [(biphenylyl)phenyltriazinyl]azathiaindenofluorene; [(biphenylyl)phenyltriazinyl]azaoxaindenofluorene; [(dimethylfluorenyl)phenyltriazinyl]azathiaindenofluorene; [(dimethylfluorenyl)phenyltriazinyl]azaoxaindenofluorene; phenyl(diphenyltriazinyl)indolocarbazole; [(biphenylyl)phenyltriazinyl]azathiaindenofluorene; phenyl[(biphenylyl)phenyltriazinyl]indolocarbazole; [(dimethylfluorenyl)phenyltriazinyl]azathiaindenofluorene; phenyl[(dimethylfluorenyl)phenyltriazinyl]indolocarbazole; (diphenyltriazinyl)azathiaindenofluorene; (diphenyltriazinyl)azathiaindenofluorene; [(biphenylyl)phenyltriazinyl]azaoxaindenofluorene; [(dimethylfluorenyl)phenyltriazinyl]azaoxaindenofluorene; [(diphenyltriazinyl)phenyl]thiaazaindenofluorene; phenyl[(diphenyltriazinyl)phenyl]indolocarbazole; [(diphenyltriazinyl)phenyl]oxazaindenofluorene; phenyl{[(dimethylfluorenyl)phenyltriazinyl]phenyl}indolocarbazole; phenyl[(diphenyltriazinyl)phenyl]dimethyldiazaindenofluorene; iridium(III)bis(phenylpyridine)(diphenylmethylpyridine); iridium(III)bis(phenylpyridine)(diphenylethylpyridine); iridium(III)bis(phenylpyridine)(diphenylisopropylpyridine); iridium(III)bis(phenylpyridine)[phenylmethyl(fluorophenyl)pyridine]; iridium(III)bis(phenylpyridine)[phenylethyl(fluorophenyl)pyridine]; iridium(III)bis(phenylpyridine)[phenylisopropyl(fluorophenyl)pyridine]; iridium(III)bis(phenylpyridine)[phenyl(methylphenyl)pyridine]; iridium(III)bis(phenylpyridine)[phenyl(ethylphenyl)pyridine]; iridium(III)bis(phenylpyridine)[phenyl(isopropylphenyl)pyridine]; iridium(III)bis(phenylpyridine)[phenyl(methylfluorophenyl)pyridine]; iridium(III)bis(phenylmethylpyridine)(diphenylmethylpyridine); iridium(III)bis(phenylmethylpyridine)(diphenylethylpyridine); iridium(III)bis(phenylmethylpyridine)(diphenylisopropylpyridine); iridium(III)bis(phenylmethylpyridine)[phenylmethyl(fluorophenyl)pyridine]; iridium(III)bis(phenylmethylpyridine)[phenylethyl(fluorophenyl)pyridine]; iridium(III)bis(phenylmethylpyridine)[phenylisopropyl(fluorophenyl)pyridine]; iridium(III)bis(phenylmethylpyridine)[phenyl(methylphenyl)pyridine]; iridium(III)bis(phenylmethylpyridine)[phenyl(ethylphenyl)pyridine]; iridium(III)bis(phenylmethylpyridine)[phenyl(isopropylphenyl)pyridine]; iridium(III)bis(phenylmethylpyridine)[phenyl(methylfluorophenyl)pyridine]; iridium(III)bis(phenylmethylpyridine)[diphenyl(methyl-d₃)pyridine]; iridium(III)bis(phenylpyridine)[diphenyl(ethyl-d₅)pyridine]; iridium(III)bis(phenylpyridine)[diphenyl(isopropyl-d₇)pyridine]; iridium(III)bis(phenylpyridine)[phenyl(methyl-d₃)(fluorophenyl)pyridine]; iridium(III)bis(phenylpyridine)[phenyl(ethyl-d₅)(fluorophenyl)pyridine]; iridium(III)bis(phenylpyridine)[phenyl(isopropyl-d₇)(fluorophenyl)pyridine]; iridium(III)bis(phenylpyridine){phenyl[(methyl-d₃)phenyl]pyridine}; iridium(III)bis(phenylpyridine){phenyl[(ethyl-d₅)phenyl]pyridine}; iridium(III)bis(phenylpyridine){phenyl[(isopropyl-d₇)phenyl]pyridine}; iridium(III)bis(phenylpyridine){phenyl[(methyl-d₃)fluorophenyl]pyridine}; iridium(III)bis[phenyl(methyl-d₃)pyridine][diphenyl(methyl-d₃)pyridine]; iridium(III)bis[phenyl(methyl-d₃)pyridine][diphenyl(ethyl-d₅)pyridine]; iridium(III)bis[phenyl(methyl-d₃)pyridine][diphenyl(isopropyl-d₇)pyridine]; iridium(III)bis[phenyl(methyl-d₃)pyridine][phenyl(methyl-d₃)(fluorophenyl)pyridine]; iridium(III)bis[phenyl(methyl-d₃)pyridine][phenyl(ethyl-d₅)(fluorophenyl)pyridine]; iridium(III)bis[phenyl(methyl-d₃)pyridine][phenyl(isopropyl-d₇)(fluorophenyl)pyridine]; iridium(III)bis[phenyl(methyl-d₃)pyridine]{phenyl[(methyl-d₃)phenyl]pyridine}; iridium(III)bis[phenyl(methyl-d₃)pyridine]{phenyl[(ethyl-d₅)phenyl]pyridine}; iridium(III)bis[phenyl(methyl-d₃)pyridine]{phenyl[(isopropyl-d₇)phenyl]pyridine}; iridium(III)bis[phenyl(methyl-d₃)pyridine]{phenyl[(methyl-d₃)fluorophenyl]pyridine}

### (Group 15A)

(diphenyltriazinyl)(dibenzofuranyl)biphenyl; (diphenyltriazinyl)(dibenzothiophenyl)biphenyl; (diphenyltriazinyl)(dibenzoselenophenyl)biphenyl; (diphenyltriazinyl)(dibenzofuranyl)terbenzene; (diphenyltriazinyl)(dibenzothiophenyl)terbenzene; (diphenyltriazinyl)(dibenzoselenophenyl)terbenzene; (diphenyltriazinyl)(phenyldibenzofuranyl)biphenyl; (diphenyltriazinyl)(phenyldibenzothiophenyl)biphenyl; (diphenyltriazinyl)(phenyldibenzoselenophenyl)biphenyl; (diphenyltriazinyl)[(biphenylyl)dibenzofuranyl]biphenyl; (diphenyltriazinyl)[(biphenylyl)dibenzothiophenyl]biphenyl; (diphenyltriazinyl)[(biphenylyl)dibenzoselenophenyl]biphenyl; (diphenyltriazinyl)[(biphenylyl)dibenzofuranyl]biphenyl; (diphenyltriazinyl)[(biphenylyl)dibenzoselenophenyl]biphenyl; (diphenyltriazinyl)[(biphenylyl)dibenzoselenophenyl]biphenyl; (diphenyltriazinyl)(phenyldibenzofuranyl)terbenzene; (diphenyltriazinyl)(phenyldibenzothiophenyl)terbenzene; (diphenyltriazinyl)(phenyldibenzoselenophenyl)terbenzene; (diphenyltriazinyl)(phenyldibenzofuranyl)terbenzene; (diphenyltriazinyl)(phenyldibenzothiophenyl)terbenzene; (diphenyltriazinyl)(phenyldibenzoselenophenyl)terbenzene; (diphenyltriazinyl)[(biphenylyl)dibenzofuranyl]terbenzene; (diphenyltriazinyl)[(biphenylyl)dibenzothiophenyl]terbenzene; (diphenyltriazinyl)[(biphenylyl)dibenzoselenophenyl]terbenzene; [(biphenylyl)phenyltriazinyl](dibenzofuranyl)biphenyl; [(biphenylyl)phenyltriazinyl](dibenzothiophenyl)biphenyl; [(biphenylyl)phenyltriazinyl](dibenzoselenophenyl)biphenyl; [(biphenylyl)phenyltriazinyl](dibenzofuranyl)biphenyl; [(biphenylyl)phenyltriazinyl](dibenzothiophenyl)biphenyl; [(biphenylyl)phenyltriazinyl](dibenzoselenophenyl)biphenyl; [di(biphenylyl)triazinyl](dibenzofuranyl)biphenyl; [di(biphenylyl)triazinyl](dibenzothiophenyl)biphenyl; [di(biphenylyl)triazinyl](dibenzoselenophenyl)biphenyl; [(dimethylfluorenyl)phenyltriazinyl](dibenzofuranyl)biphenyl; [(dimethylfluorenyl)phenyltriazinyl](dibenzothiophenyl)biphenyl; [(dimethylfluorenyl)phenyltriazinyl](dibenzoselenophenyl)biphenyl; [(biphenylyl)phenyltriazinyl](phenyldibenzofuranyl)biphenyl; [(biphenylyl)phenyltriazinyl](phenyldibenzothiophenyl)biphenyl; [(biphenylyl)phenyltriazinyl](phenyldibenzoselenophenyl)biphenyl; [(biphenylyl)phenyltriazinyl](phenyldibenzofuranyl)biphenyl; [(biphenylyl)phenyltriazinyl](phenyldibenzothiophenyl)biphenyl; [(biphenylyl)phenyltriazinyl](phenyldibenzoselenophenyl)biphenyl; [(dimethylfluorenyl)phenyltriazinyl](phenyldibenzofuranyl)biphenyl; [(dimethylfluorenyl)phenyltriazinyl](phenyldibenzofuranyl)biphenyl; [(dimethylfluorenyl)phenyltriazinyl](phenyldibenzofuranyl)biphenyl; [(biphenylyl)phenyltriazinyl](dibenzofuranyl)terbenzene; [(biphenylyl)phenyltriazinyl](dibenzothiophenyl)terbenzene; [(biphenylyl)phenyltriazinyl](dibenzoselenophenyl)terbenzene; [(biphenylyl)phenyltriazinyl](dibenzofuranyl)terbenzene; [(biphenylyl)phenyltriazinyl](dibenzothiophenyl)terbenzene; [(biphenylyl)phenyltriazinyl](dibenzoselenophenyl)terbenzene; [(dimethylfluorenyl)phenyltriazinyl](dibenzofuranyl)terbenzene; [(dimethylfluorenyl)phenyltriazinyl](dibenzofuranyl)terbenzene; [(dimethylfluorenyl)phenyltriazinyl](dibenzofuranyl)terbenzene; [(biphenylyl)phenyltriazinyl][(biphenylyl)dibenzofuranyl]biphenyl; [(biphenylyl)phenyltriazinyl][(biphenylyl)dibenzothiophenyl]biphenyl; [(biphenylyl)phenyltriazinyl][(biphenylyl)dibenzoselenophenyl]biphenyl; [(biphenylyl)phenyltriazinyl][(biphenylyl)dibenzofuranyl]biphenyl; [(biphenylyl)phenyltriazinyl][(biphenylyl)dibenzothiophenyl]biphenyl; [(biphenylyl)phenyltriazinyl][(biphenylyl)dibenzoselenophenyl]biphenyl; [(dimethylfluorenyl)phenyltriazinyl][(biphenylyl)dibenzofuranyl]biphenyl; [(dimethylfluorenyl)phenyltriazinyl][(biphenylyl)dibenzothiophenyl]biphenyl; [(dimethylfluorenyl)phenyltriazinyl][(biphenylyl)dibenzoselenophenyl]biphenyl; (diphenyltriazinyl)(dibenzofuranyl)terbenzene; (diphenyltriazinyl)(dibenzothiophenyl)terbenzene; (diphenyltriazinyl)(dibenzoselenophenyl)terbenzene; (dibenzofuranyl)(diphenyltriazinyl)terbenzene; (dibenzothiophenyl)(diphenyltriazinyl)terbenzene; (diphenyltriazinyl)(dibenzoselenophenyl)terbenzene; (diphenyltriazinyl)(dibenzofuranyl)biphenyl; (diphenyltriazinyl)(dibenzothiophenyl)biphenyl; (diphenyltriazinyl)(dibenzoselenophenyl)biphenyl; (diphenyltriazinyl)(dibenzofuranyl)biphenyl; (diphenyltriazinyl)(dibenzothiophenyl)biphenyl; (diphenyltriazinyl)(dibenzoselenophenyl)biphenyl; (diphenyltriazinyl)(dibenzofuranyl)biphenyl; (diphenyltriazinyl)(dibenzothiophenyl)biphenyl; (diphenyltriazinyl)(dibenzoselenophenyl)biphenyl; (diphenyltriazinyl)(dibenzofuranyl)terbenzene; (diphenyltriazinyl)(dibenzothiophenyl)terbenzene; (diphenyltriazinyl)(dibenzoselenophenyl)terbenzene; (diphenyltriazinyl)(dibenzofuranyl)terbenzene; (diphenyltriazinyl)(dibenzothiophenyl)terbenzene; (diphenyltriazinyl)(dibenzoselenophenyl)terbenzene; (diphenyltriazinyl)(dibenzofuranyl)terbenzene; (diphenyltriazinyl)(dibenzothiophenyl)terbenzene; (diphenyltriazinyl)(dibenzoselenophenyl)terbenzene; (diphenyltriazinyl)(diphenyldibenzofuranyl)biphenyl; (diphenyltriazinyl)(diphenyldibenzothiophenyl)biphenyl; (diphenyltriazinyl)(diphenyldibenzoselenophenyl)biphenyl; (diphenyltriazinyl)(diphenyldibenzofuranyl)biphenyl; (diphenyltriazinyl)(diphenyldibenzothiophenyl)biphenyl; (diphenyltriazinyl)(diphenyldibenzoselenophenyl)biphenyl; (diphenyltriazinyl)(phenyldibenzofuranyl)biphenyl; (diphenyltriazinyl)(phenyldibenzothiophenyl)biphenyl; (diphenyltriazinyl)(phenyldibenzoselenophenyl)biphenyl; (diphenyltriazinyl)(pyridinyl)(phenyldibenzofuranyl)benzene; (diphenyltriazinyl)(pyridinyl)(phenyldibenzothiophenyl)benzene; (diphenyltriazinyl)(pyridinyl)(phenyldibenzoselenophenyl)benzene; (diphenyltriazinyl)(pyridinyl)(phenyldibenzofuranyl)benzene; (diphenyltriazinyl)(pyridinyl)(phenyldibenzothiophenyl)benzene; (diphenyltriazinyl)(pyridinyl)(phenyldibenzoselenophenyl)benzene; (diphenyltriazinyl)[(pyridinyl)dibenzofuranyl]biphenyl; (diphenyltriazinyl)[(pyridinyl)dibenzothiophenyl]biphenyl; (diphenyltriazinyl)[(pyridinyl)dibenzoselenophenyl]biphenyl; (diphenyltriazinyl)(dibenzofuranyl)terbenzene; (diphenyltriazinyl)(dibenzothiophenyl)terbenzene; (diphenyltriazinyl)(dibenzoselenophenyl)terbenzene; (dibenzofuranyl)(diphenyltriazinyl)terbenzene; (dibenzothiophenyl)(diphenyltriazinyl)terbenzene; (dibenzoselenophenyl)(diphenyltriazinyl)terbenzene; (diphenyltriazinyl)(dibenzofuranyl)(dimethylfluorenyl)benzene; (diphenyltriazinyl)(dibenzothiophenyl)(dimethylfluorenyl)benzene; (diphenyltriazinyl)(dibenzoselenophenyl)(dimethylfluorenyl)benzene; (diphenyltriazinyl)(dimethylfluorenyl)biphenyl; (diphenyltriazinyl)[spiro(cyclopentanefluorenyl)]biphenyl; (diphenyltriazinyl)(dimethylfluorenyl)terbenzene; (diphenyltriazinyl)(diphenylfluorenyl)biphenyl; [(diphenylyl)phenyltriazinyl](dimethylfluorenyl)biphenyl; [(dimethylfluorenyl)phenyltriazinyl](dimethylfluorenyl)terbenzene; (diphenyltriazinyl)di(dimethylfluorenyl)benzene; (diphenyltriazinyl)(dimethylfluorenyl)terbenzene; (diphenyltriazinyl)phenyldibenzofuran; (diphenyltriazinyl)phenyldibenzothiophene; (diphenyltriazinyl)phenyldibenzoselenophene; [(biphenylyl)phenyltriazinyl]phenyldibenzofuran; [(biphenylyl)phenyltriazinyl]phenyldibenzothiophene; [(biphenylyl)phenyltriazinyl]phenyldibenzoselenophene; [(biphenylyl)phenyltriazinyl]phenyldibenzofuran; [(biphenylyl)phenyltriazinyl]phenyldibenzothiophene; [(biphenylyl)phenyltriazinyl]phenyldibenzoselenophene; [di(biphenylyl)triazinyl]phenyldibenzofuran; [di(biphenylyl)triazinyl]phenyldibenzothiophene; [di(biphenylyl)triazinyl]phenyldibenzoselenophene; [di(biphenylyl)triazinyl]phenyldibenzofuran; [di(biphenylyl)triazinyl]phenyldibenzothiophene; [di(biphenylyl)triazinyl]phenyldibenzoselenophene; [di(biphenylyl)triazinyl]phenyldibenzofuran; [di(biphenylyl)triazinyl]phenyldibenzothiophene; [di(biphenylyl)triazinyl]phenyldibenzoselenophene; (diphenyltriazinyl)(phenyldibenzofuranyl)benzene; (diphenyltriazinyl)(phenyldibenzothiophenyl)benzene; (diphenyltriazinyl)(phenyldibenzoselenophenyl)benzene; [(biphenylyl)phenyltriazinyl](phenyldibenzofuranyl)benzene; [(biphenylyl)phenyltriazinyl](phenyldibenzothiophenyl)benzene; [(biphenylyl)phenyltriazinyl](phenyldibenzoselenophenyl)benzene; [(biphenylyl)phenyltriazinyl](phenyldibenzofuranyl)benzene; [(biphenylyl)phenyltriazinyl](phenyldibenzothiophenyl)benzene; [(biphenylyl)phenyltriazinyl](phenyldibenzoselenophenyl)benzene; (diphenyltriazinyl)(phenyldibenzofuranyl)benzene; (diphenyltriazinyl)(phenyldibenzothiophenyl)benzene; (diphenyltriazinyl)(phenyldibenzoselenophenyl)benzene; [phenyl(biphenylyl)triazinyl](phenyldibenzofuranyl)benzene; [phenyl(biphenylyl)triazinyl](phenyldibenzothiophenyl)benzene; [phenyl(biphenylyl)triazinyl](phenyldibenzoselenophenyl)benzene; [phenyl(biphenylyl)triazinyl](phenyldibenzofuranyl)benzene; [phenyl(biphenylyl)triazinyl](phenyldibenzothiophenyl)benzene; [phenyl(biphenylyl)triazinyl](phenyldibenzoselenophenyl)benzene; (diphenyltriazinyl)(phenyldibenzofuranyl)biphenyl; (diphenyltriazinyl)(phenyldibenzothiophenyl)biphenyl; (diphenyltriazinyl)(phenyldibenzoselenophenyl)biphenyl; [(biphenylyl)phenyltriazinyl](phenyldibenzofuranyl)biphenyl; [(biphenylyl)phenyltriazinyl](phenyldibenzothiophenyl)biphenyl; [(biphenylyl)phenyltriazinyl](phenyldibenzoselenophenyl)biphenyl; [(biphenylyl)phenyltriazinyl](phenyldibenzofuranyl)biphenyl; [(biphenylyl)phenyltriazinyl](phenyldibenzothiophenyl)biphenyl; [(biphenylyl)phenyltriazinyl](phenyldibenzoselenophenyl)biphenyl; (phenyldibenzofuranyl)(diphenyltriazinyl)biphenyl; (phenyldibenzothiophenyl)(diphenyltriazinyl)biphenyl; (phenyldibenzoselenophenyl)(diphenyltriazinyl)biphenyl; (phenyldibenzothiophenyl)[phenyl(biphenylyl)triazinyl]biphenyl; (phenyldibenzoselenophenyl)[phenyl(biphenylyl)triazinyl]biphenyl; (phenyldibenzofuranyl)[phenyl(biphenylyl)triazinyl]biphenyl; (phenyldibenzothiophenyl)[phenyl(biphenylyl)triazinyl]biphenyl; (phenyldibenzoselenophenyl)[phenyl(biphenylyl)triazinyl]biphenyl; (diphenyltriazinyl)(phenyldibenzofuranyl)biphenyl; (diphenyltriazinyl)(phenyldibenzothiophenyl)biphenyl; (diphenyltriazinyl)(phenyldibenzoselenophenyl)biphenyl; [phenyl(biphenylyl)triazinyl](phenyldibenzofuranyl)biphenyl; [phenyl(biphenylyl)triazinyl](phenyldibenzothiophenyl)biphenyl; [phenyl(biphenylyl)triazinyl](phenyldibenzoselenophenyl)biphenyl; [phenyl(biphenylyl)triazinyl](phenyldibenzofuranyl)biphenyl; [phenyl(biphenylyl)triazinyl](phenyldibenzothiophenyl)biphenyl; [phenyl(biphenylyl)triazinyl](phenyldibenzoselenophenyl)biphenyl; (biphenylyl)(diphenyltriazinyl)dibenzofuran; (biphenylyl)(diphenyltriazinyl)dibenzothiophene; (biphenylyl)(diphenyltriazinyl)dibenzoselenophene; (biphenylyl)[(biphenylyl)phenyltriazinyl]dibenzofuran; (biphenylyl)[(biphenylyl)phenyltriazinyl]dibenzothiophene; (biphenylyl)[(biphenylyl)phenyltriazinyl]dibenzoselenophene; (biphenylyl)[(biphenylyl)phenyltriazinyl]dibenzofuran; (biphenylyl)[(biphenylyl)phenyltriazinyl]dibenzothiophene; (biphenylyl)[(biphenylyl)phenyltriazinyl]dibenzoselenophene; (biphenylyl)[(diphenyltriazinyl)phenyl]dibenzofuran; (biphenylyl)[(diphenyltriazinyl)phenyl]dibenzothiophene; (biphenylyl)[(diphenyltriazinyl)phenyl]dibenzoselenophene; (biphenylyl){[(biphenylyl)phenyltriazinyl]phenyl}dibenzofuran; (biphenylyl){[(biphenylyl)phenyltriazinyl]phenyl}dibenzothiophene; (biphenylyl){[(biphenylyl)phenyltriazinyl]phenyl}dibenzoselenophene; (biphenylyl){[(biphenylyl)phenyltriazinyl]phenyl}dibenzofuran; (biphenylyl){[(biphenylyl)phenyltriazinyl]phenyl}dibenzothiophene; (biphenylyl){[(biphenylyl)phenyltriazinyl]phenyl}dibenzoselenophene; (biphenylyl)[(diphenyltriazinyl)phenyl]dibenzofuran; (biphenylyl)[(diphenyltriazinyl)phenyl]dibenzothiophene; (biphenylyl)[(diphenyltriazinyl)phenyl]dibenzoselenophene; (biphenylyl){[phenyl(biphenylyl)triazinyl]phenyl}dibenzofuran; (biphenylyl){[phenyl(biphenylyl)triazinyl]phenyl}dibenzothiophene; (biphenylyl){[phenyl(biphenylyl)triazinyl]phenyl}dibenzoselenophene; (biphenylyl){[phenyl(biphenylyl)triazinyl]phenyl}dibenzofuran; (biphenylyl){[phenyl(biphenylyl)triazinyl]phenyl}dibenzothiophene; (biphenylyl){[phenyl(biphenylyl)triazinyl]phenyl}dibenzoselenophene; (biphenylyl)(diphenyltriazinyl)dibenzofuran; (biphenylyl)(diphenyltriazinyl)dibenzothiophene; (biphenylyl)(diphenyltriazinyl)dibenzoselenophene; (biphenylyl)[(biphenylyl)phenyltriazinyl]dibenzofuran; (biphenylyl)[(biphenylyl)phenyltriazinyl]dibenzothiophene; (biphenylyl)[(biphenylyl)phenyltriazinyl]dibenzoselenophene; (biphenylyl)[(biphenylyl)phenyltriazinyl]dibenzofuran; (biphenylyl)[(biphenylyl)phenyltriazinyl]dibenzothiophene; (biphenylyl)[(biphenylyl)phenyltriazinyl]dibenzoselenophene; (biphenylyl)[(diphenyltriazinyl)phenyl]dibenzofuran; (biphenylyl)[(diphenyltriazinyl)phenyl]dibenzothiophene; (biphenylyl)[(diphenyltriazinyl)phenyl]dibenzoselenophene; (biphenylyl){[(biphenylyl)phenyltriazinyl]phenyl}dibenzofuran; (biphenylyl){[(biphenylyl)phenyltriazinyl]phenyl}dibenzothiophene; (biphenylyl){[(biphenylyl)phenyltriazinyl]phenyl}dibenzoselenophene; (biphenylyl){[phenyl(biphenylyl)triazinyl]phenyl}dibenzofuran; (biphenylyl){[phenyl(biphenylyl)triazinyl]phenyl}dibenzothiophene; (biphenylyl){[phenyl(biphenylyl)triazinyl]phenyl}dibenzoselenophene; (biphenylyl)[(diphenyltriazinyl)phenyl]dibenzofuran; (biphenylyl)[(diphenyltriazinyl)phenyl]dibenzothiophene; (biphenylyl)[(diphenyltriazinyl)phenyl]dibenzoselenophene; (biphenylyl){[(biphenylyl)phenyltriazinyl]phenyl}dibenzofuran; (biphenylyl){[(biphenylyl)phenyltriazinyl]phenyl}dibenzothiophene; (biphenylyl){[(biphenylyl)phenyltriazinyl]phenyl}dibenzoselenophene; (biphenylyl){[(biphenylyl)phenyltriazinyl]phenyl}dibenzofuran; (biphenylyl){[(biphenylyl)phenyltriazinyl]phenyl}dibenzothiophene; (biphenylyl){[(biphenylyl)phenyltriazinyl]phenyl}dibenzoselenophene; phenyl(dimethylfluorenyl)(phenyldibenzofuranyl)triazine; phenyl(dimethylfluorenyl)(phenyldibenzothiophenyl)triazine; phenyl(dimethylfluorenyl)(phenyldibenzoselenophenyl)triazine; (biphenylyl)(dimethylfluorenyl)(phenyldibenzofuranyl)triazine; (biphenylyl)(dimethylfluorenyl)(phenyldibenzothiophenyl)triazine; (biphenylyl)(dimethylfluorenyl)(phenyldibenzoselenophenyl)triazine; (biphenylyl)(dimethylfluorenyl)(phenyldibenzofuranyl)triazine; (biphenylyl)(dimethylfluorenyl)(phenyldibenzothiophenyl)triazine; (biphenylyl)(dimethylfluorenyl)(phenyldibenzoselenophenyl)triazine; phenyl(dimethylfluorenyl)[(phenyldibenzofuranyl)phenyl]triazine; phenyl(dimethylfluorenyl)[(phenyldibenzothiophenyl)phenyl]triazine; phenyl(dimethylfluorenyl)[(phenyldibenzoselenophenyl)phenyl]triazine; bis(dimethylfluorenyl)[(phenyldibenzofuranyl)phenyl]triazine; bis(dimethylfluorenyl)[(phenyldibenzothiophenyl)phenyl]triazine; bis(dimethylfluorenyl)[(phenyldibenzoselenophenyl)phenyl]triazine; phenyl[(biphenylyl)dibenzofuranyl](dimethylfluorenyl)triazine; phenyl[(biphenylyl)dibenzothiophenyl](dimethylfluorenyl)triazine; phenyl[(biphenylyl)dibenzoselenophenyl](dimethylfluorenyl)triazine; phenyl[(biphenylyl)dibenzofuranyl](dimethylfluorenyl)triazine; phenyl[(biphenylyl)dibenzothiophenyl](dimethylfluorenyl)triazine; phenyl[(biphenylyl)dibenzoselenophenyl](dimethylfluorenyl)triazine; phenyl(dimethylfluorenyl){[(biphenylyl)dibenzofuranyl]phenyl}triazine; phenyl(dimethylfluorenyl){[(biphenylyl)dibenzothiophenyl]phenyl}triazine; phenyl(dimethylfluorenyl){[(biphenylyl)dibenzoselenophenyl]phenyl}triazine; phenyl(dimethylfluorenyl){[(biphenylyl)dibenzofuranyl]phenyl}triazine; phenyl(dimethylfluorenyl){[(biphenylyl)dibenzothiophenyl]phenyl}triazine; phenyl(dimethylfluorenyl){[(biphenylyl)dibenzoselenophenyl]phenyl}triazine; phenyl[(dimethylfluorenyl)phenyl](phenyldibenzofuranyl)triazine; phenyl[(dimethylfluorenyl)phenyl](phenyldibenzothiophenyl)triazine; phenyl[(dimethylfluorenyl)phenyl](phenyldibenzoselenophenyl)triazine; phenyl(dimethylfluorenyl)(phenyldibenzofuranyl)triazine; phenyl(dimethylfluorenyl)(phenyldibenzothiophenyl)triazine; phenyl(dimethylfluorenyl)(phenyldibenzoselenophenyl)triazine; phenyl[(biphenylyl)dibenzofuranyl](dimethylfluorenyl)triazine; phenyl[(biphenylyl)dibenzothiophenyl](dimethylfluorenyl)triazine; phenyl[(biphenylyl)dibenzofuranyl](dimethylfluorenyl)triazine; phenyl(dimethylfluorenyl){[(biphenylyl)dibenzofuranyl]phenyl}triazine; phenyl(dimethylfluorenyl){[(biphenylyl)dibenzothiophenyl]phenyl}triazine; phenyl(dimethylfluorenyl){[(biphenylyl)dibenzoselenophenyl]phenyl}triazine; phenyl[(dimethylfluorenyl)phenyl](phenyldibenzofuranyl)triazine; phenyl[(dimethylfluorenyl)phenyl](phenyldibenzothiophenyl)triazine; phenyl[(dimethylfluorenyl)phenyl](phenyldibenzoselenophenyl)triazine; phenyl(dimethylfluorenyl)(phenyldibenzofuranyl)triazine; phenyl(dimethylfluorenyl)(phenyldibenzothiophenyl)triazine; phenyl(dimethylfluorenyl)(phenyldibenzoselenophenyl)triazine; phenyl(diphenyltriazinyl)dibenzofuran; phenyl(diphenyltriazinyl)dibenzothiophene; phenyl(diphenyltriazinyl)dibenzoselenophene; phenyl[(diphenyltriazinyl)phenyl]dibenzofuran; phenyl[(diphenyltriazinyl)phenyl]dibenzothiophene; phenyl[(diphenyltriazinyl)phenyl]dibenzoselenophene; (biphenylyl)[(diphenyltriazinyl)phenyl]dibenzofuran; (biphenylyl)[(diphenyltriazinyl)phenyl]dibenzothiophene; (biphenylyl)[(diphenyltriazinyl)phenyl]dibenzoselenophene; diphenyl[(diphenyltriazinyl)phenyl]dibenzofuran; diphenyl[(diphenyltriazinyl)phenyl]dibenzothiophene; diphenyl[(diphenyltriazinyl)phenyl]dibenzoselenophene; phenyl(diphenyltriazinyl)dibenzofuran; phenyl(diphenyltriazinyl)dibenzothiophene; phenyl(diphenyltriazinyl)dibenzoselenophene; (biphenylyl)(diphenyltriazinyl)dibenzofuran; (biphenylyl)(diphenyltriazinyl)dibenzothiophene; (biphenylyl)(diphenyltriazinyl)dibenzoselenophene; (biphenylyl)(diphenyltriazinyl)dibenzofuran; (biphenylyl)(diphenyltriazinyl)dibenzothiophene; (biphenylyl)(diphenyltriazinyl)dibenzoselenophene; diphenyl[(diphenyltriazinyl)phenyl]dibenzofuran; diphenyl[(diphenyltriazinyl)phenyl]dibenzothiophene; diphenyl[(diphenyltriazinyl)phenyl]dibenzoselenophene; (biphenylyl)[(diphenyltriazinyl)phenyl]dibenzofuran; (biphenylyl)[(diphenyltriazinyl)phenyl]dibenzothiophene; (biphenylyl)[(diphenyltriazinyl)phenyl]dibenzoselenophene; (diphenyltriazinyl)phenyldibenzofuran; (diphenyltriazinyl)phenyldibenzothiophene; (diphenyltriazinyl)phenyldibenzoselenophene; [(diphenyltriazinyl)phenyl]phenyldibenzofuran; [(diphenyltriazinyl)phenyl]phenyldibenzothiophene; [(diphenyltriazinyl)phenyl]phenyldibenzoselenophene; [(diphenyltriazinyl)phenyl]phenyldibenzofuran; [(diphenyltriazinyl)phenyl]phenyldibenzothiophene; [(diphenyltriazinyl)phenyl]phenyldibenzoselenophene; [(diphenyltriazinyl)phenyl](biphenylyl)dibenzofuran; [(diphenyltriazinyl)phenyl](biphenylyl)dibenzothiophene; [(diphenyltriazinyl)phenyl](biphenylyl)dibenzoselenophene; [(diphenyltriazinyl)phenyl](biphenylyl)dibenzofuran; [(diphenyltriazinyl)phenyl](biphenylyl)dibenzothiophene; [(diphenyltriazinyl)phenyl](biphenylyl)dibenzoselenophene; [(diphenyltriazinyl)phenyl]phenyldibenzofuran; [(diphenyltriazinyl)phenyl]phenyldibenzothiophene; [(diphenyltriazinyl)phenyl]phenyldibenzoselenophene; (diphenyltriazinyl)(phenyldibenzofuranyl)pyridine; (diphenyltriazinyl)(phenyldibenzothiophenyl)pyridine; (diphenyltriazinyl)(phenyldibenzoselenophenyl)pyridine; [phenyl(phenylpyridinyl)triazinyl](phenyldibenzofuranyl)pyridine; [phenyl(phenylpyridinyl)triazinyl](phenyldibenzothiophenyl)pyridine; [phenyl(phenylpyridinyl)triazinyl](phenyldibenzoselenophenyl)pyridine; {[phenyl(phenylpyridinyl)triazinyl]phenyl}phenyldibenzofuran; {[phenyl(phenylpyridinyl)triazinyl]phenyl}phenyldibenzothiophene; {[phenyl(phenylpyridinyl)triazinyl]phenyl}phenyldibenzoselenophene; (diphenyltriazinyl)[(biphenylyl)dibenzofuranyl]pyridine; (diphenyltriazinyl)[(biphenylyl)dibenzothiophenyl]pyridine; (diphenyltriazinyl)[(biphenylyl)dibenzoselenophenyl]pyridine; phenyl(dimethylindenopyridinyl)(phenyldibenzofuranyl)triazine; phenyl(dimethylindenopyridinyl)(phenyldibenzothiophenyl)triazine; phenyl(dimethylindenopyridinyl)(phenyldibenzoselenophenyl)triazine; (biphenylyl)(dimethylindenopyridinyl)(phenyldibenzofuranyl)triazine; (biphenylyl)(dimethylindenopyridinyl)(phenyldibenzothiophenyl)triazine; (biphenylyl)(dimethylindenopyridinyl)(phenyldibenzoselenophenyl)triazine; phenyl(dimethylindenopyridinyl)(phenyldibenzofuranyl)triazine; phenyl(dimethylindenopyridinyl)(phenyldibenzothiophenyl)triazine; phenyl(dimethylindenopyridinyl)(phenyldibenzoselenophenyl)triazine; phenyl(spiro[cyclohexanefluorene]yl)(phenyldibenzofuranyl)triazine; phenyl(spiro[cyclohexanefluorene]yl)(phenyldibenzothiophenyl)triazine; phenyl(spiro[cyclohexanefluorene]yl)(phenyldibenzoselenophenyl)triazine; phenyl(spirobi[fluorene]yl)(phenyldibenzofuranyl)triazine; phenyl(spirobi[fluorene]yl)(phenyldibenzothiophenyl)triazine; phenyl(spirobi[fluorene]yl)(phenyldibenzoselenophenyl)triazine; (diphenyl-d₁₀)[(phenyldibenzofuranyl)phenyl-d₁₄]triazine; (diphenyl-d₁₀)[(phenyldibenzothiophenyl)phenyl-d₁₄]triazine; (diphenyl-d₁₀)[(phenyldibenzoselenophenyl)phenyl-d₁₄]triazine(diphenyl-d₁₀){[(biphenylyl)dibenzofuranyl]phenyl-d₁₈}triazine; (diphenyl-d₁₀){[(biphenylyl)dibenzothiophenyl]phenyl-d₁₈}triazine; (diphenyl-d₁₀){[(biphenylyl)dibenzoselenophenyl]phenyl-d₁₈}triazinephenyl[(dimethyl-d₆)fluorenyl](phenyldibenzofuranyl)triazine; phenyl[(dimethyl-d₆)fluorenyl](phenyldibenzothiophenyl)triazine; phenyl[(dimethyl-d₆)fluorenyl](phenyldibenzoselenophenyl)triazine; (diphenyl-d₁₀){[(biphenylyl)dibenzofuranyl]phenyl-d₁₈}triazine; (diphenyl-d₁₀){[(biphenylyl)dibenzothiophenyl]phenyl-d₁₈}triazine; (diphenyl-d₁₀){[(biphenylyl)dibenzoselenophenyl]phenyl-d₁₈}triazine(diphenyl-d₁₀){[(biphenylyl)dibenzofuranyl]phenyl}triazine; (diphenyl-d₁₀){[(biphenylyl)dibenzothiophenyl]phenyl}triazine; (diphenyl-d₁₀){[(biphenylyl)dibenzoselenophenyl]phenyl}triazine; (diphenyltriazinyl)[(biphenylyl)dibenzofuranyl]biphenyl; (diphenyltriazinyl)[(biphenylyl)dibenzothiophenyl]biphenyl; (diphenyltriazinyl)[(biphenylyl)dibenzoselenophenyl]biphenyl; (diphenyltriazinyl)[(biphenylyl)dibenzofuranyl]biphenyl; (diphenyltriazinyl)[(biphenylyl)dibenzothiophenyl]biphenyl; (diphenyltriazinyl)[(biphenylyl)dibenzoselenophenyl]biphenyl; [di(fluorophenyl)triazinyl][(biphenylyl)dibenzofuranyl]phenyl; [di(fluorophenyl)triazinyl][(biphenylyl)dibenzothiophenyl]phenyl; [di(fluorophenyl)triazinyl][(biphenylyl)dibenzoselenophenyl]phenyl; (dibenzothiophenyl)(diphenyltriazinyl)biphenyl; (diphenyltriazinyl)(dibenzothiophenyl)biphenyl; (diphenyltriazinyl)(dibenzothiophenyl)biphenyl; (dibenzofuranyl)triphenylene; (dibenzothiophenyl)triphenylene; (dibenzoselenophenyl)triphenylene; (dibenzofuranyl)(triphenylenyl)benzene; (dibenzothiophenyl)(triphenylenyl)benzene; (dibenzoselenophenyl)(triphenylenyl)benzene; (phenyldibenzofuranyl)(triphenylenyl)benzene; (phenyldibenzothiophenyl)(triphenylenyl)benzene; (phenyldibenzoselenophenyl)(triphenylenyl)benzene; (dibenzofuranyl)(triphenylenyl)biphenyl; (dibenzothiophenyl)(triphenylenyl)biphenyl; (dibenzoselenophenyl)(triphenylenyl)biphenyl; phenyl(triphenylenyl)dibenzofuran; phenyl(triphenylenyl)dibenzothiophene; phenyl(triphenylenyl)dibenzoselenophene; (biphenylyl)(triphenylenyl)dibenzofuran; (biphenylyl)(triphenylenyl)dibenzothiophene; (biphenylyl)(triphenylenyl)dibenzoselenophene; (terbenzenyl)(triphenylenyl)dibenzofuran; (terbenzenyl)(triphenylenyl)dibenzothiophene; (terbenzenyl)(triphenylenyl)dibenzoselenophene; (biphenylyl)[(triphenylenyl)phenyl]dibenzofuran; (biphenylyl)[(triphenylenyl)phenyl]dibenzothiophene; (biphenylyl)[(triphenylenyl)phenyl]dibenzoselenophene; (carbazolyl)(triphenylenyl)dibenzofuran; (carbazolyl)(triphenylenyl)dibenzothiophene; (carbazolyl)(triphenylenyl)dibenzoselenophene; (carbazolyl)(triphenylenyl)biphenyl; (triphenylenyl)(carbazolyl)biphenyl; (carbazolyl)[(triphenylenyl)phenyl]pyridine; tri(terbenzenyl)triazine; di(terbenzenyl)(biphenylyl)triazine; di(terbenzenyl)(biphenylyl)triazine; di(terbenzenyl)(dimethylfluorenyl)triazine; (dimethylfluorenyl)(terbenzenyl)(biphenylyl)triazine; phenyl(dibenzofuranyl)(terbenzenyl)triazine; phenyl(dibenzothiophenyl)(terbenzenyl)triazine; phenyl(dibenzoselenophenyl)(terbenzenyl)triazine; (biphenylyl)(dibenzofuranyl)(terbenzenyl)triazine; (biphenylyl)(dibenzothiophenyl)(terbenzenyl)triazine; (biphenylyl)(dibenzoselenophenyl)(terbenzenyl)triazine; (dibenzofuranyl)(dimethylfluorenyl)(terbenzenyl)triazine; (dibenzothiophenyl)(dimethylfluorenyl)(terbenzenyl)triazine; (dibenzoselenophenyl)(dimethylfluorenyl)(terbenzenyl)triazine; [(carbazolyl)phenyl](terphenylyl)carbazole; [(carbazolyl)phenyl](terphenylyl)carbazole; [(carbazolyl)pyridinyl](terphenylyl)carbazole; [(carbazolyl)pyridinyl](terphenylyl)carbazole; [(carbazolyl)pyridinyl](terphenylyl)carbazole; (terphenylyl)bicarbazole; (terphenylyl)bicarbazole; [(carbazolyl)phenyl](triphenylenyl)carbazole; [(carbazolyl)phenyl](triphenylenyl)carbazole; [(carbazolyl)pyridinyl](triphenylenyl)carbazole; [(carbazolyl)pyridinyl](triphenylenyl)carbazole; [(carbazolyl)phenyl](triphenylenyl)carbazole; (triphenylenyl)bicarbazole; (triphenylenyl)bicarbazole; [(triphenylenyl)phenyl]bicarbazole; [(triphenylenyl)phenyl]bicarbazole; [(carbazolyl)phenyl](dimethylfluorenyl)carbazole; [(carbazolyl)phenyl](dimethylfluorenyl)carbazole; [(carbazolyl)pyridinyl](dimethylfluorenyl)carbazole; [(carbazolyl)pyridinyl](dimethylfluorenyl)carbazole; (dimethylfluorenyl)bicarbazole; (dimethylfluorenyl) bicarbazole; [c-(dimethylfluorenyl)phenyl]bicarbazole; [c-(dimethylfluorenyl)phenyl]bicarbazole; [(carbazolyl)phenyl](dibenzofuranyl)carbazole; [(carbazolyl)phenyl](dibenzothiophenyl)carbazole; [(carbazolyl)phenyl](dibenzoselenophenyl)carbazole; [(carbazolyl)phenyl](dibenzofuranyl)carbazole; [(carbazolyl)phenyl](dibenzothiophenyl)carbazole; [(carbazolyl)phenyl](dibenzoselenophenyl)carbazole; [(carbazolyl)phenyl](dibenzofuranyl)carbazole; [(carbazolyl)phenyl](dibenzothiophenyl)carbazole; [(carbazolyl)phenyl](dibenzoselenophenyl)carbazole; [(carbazolyl)phenyl](dibenzofuranyl)carbazole; [(carbazolyl)phenyl](dibenzothiophenyl)carbazole; [(carbazolyl)phenyl](dibenzoselenophenyl)carbazole; (dibenzofuranyl)bicarbazole; (dibenzothiophenyl)bicarbazole; (dibenzoselenophenyl)bicarbazole; (dibenzofuranyl)bicarbazole; (dibenzothiophenyl)bicarbazole; (dibenzoselenophenyl)bicarbazole; (dibenzofuranyl)bicarbazole; (dibenzothiophenyl)bicarbazole; (dibenzoselenophenyl)bicarbazole; (dibenzofuranyl)bicarbazole; (dibenzothiophenyl)bicarbazole; (dibenzoselenophenyl)bicarbazole; (dibenzofuranyl)phenylbicarbazole; (dibenzothiophenyl)phenylbicarbazole; (dibenzoselenophenyl)phenylbicarbazole; (dibenzofuranyl)(bicarbazolyl)biphenyl; (dibenzothiophenyl)(bicarbazolyl)biphenyl; (dibenzoselenophenyl)(bicarbazolyl)biphenyl; (dibenzofuranyl)(bicarbazolyl)biphenyl; (dibenzothiophenyl)(bicarbazolyl)biphenyl; (dibenzoselenophenyl)(bicarbazolyl)biphenyl; (dibenzofuranyl)phenylbicarbazole; (dibenzothiophenyl)phenylbicarbazole; (dibenzoselenophenyl)phenylbicarbazole; (dibenzofuranyl)(bicarbazolyl)biphenyl; (dibenzothiophenyl)(bicarbazolyl)biphenyl; (dibenzoselenophenyl)(bicarbazolyl)biphenyl; (dibenzofuranyl)(bicarbazolyl)biphenyl; (dibenzothiophenyl)(bicarbazolyl)biphenyl; (dibenzoselenophenyl)(bicarbazolyl)biphenyl

### (Group 16A)

diphenylindolocarbazole; phenyl(biphenylyl)indolocarbazole; di(biphenylyl)indolocarbazole; phenyl(terphenylyl)indolocarbazole; phenyl(quaterphenylyl)indolocarbazole; phenyl(quinquephenylyl)indolocarbazole; phenyl(naphthalenyl)indolocarbazole; phenyl(binaphthalenyl)indolocarbazole; phenyl(acenaphthenyl)indolocarbazole; phenyl(acenaphthenyl)indolocarbazole; phenyl(azulenylphenyl)indolocarbazole; phenyl[(phenylanthracenyl)phenyl]indolocarbazole; phenyl(methylphenyl)indolocarbazole; phenyl[(diphenylamino)phenyl]indolocarbazole; phenyl[bis(trimethylphenyl)borane]phenyl]indolocarbazole; di(biphenylyl)indolocarbazole; biphenyl(pyridinylphenyl)indolocarbazole; phenyl(triphenylenyl)indolocarbazole; phenyl(terphenylyl)indolocarbazole; phenyl(chrysenyl)indolocarbazole; phenyl(pyrenyl)indolocarbazole; phenyl(phenanthrenyl)indolocarbazole; phenyl(coronenyl)indolocarbazole; phenyl[(diphenylpyrimidinyl)phenyl]indolocarbazole; phenyl[(diphenyltriazinyl)phenyl]indolocarbazole; phenyl[(bithiophenyl)phenyl]indolocarbazole; phenyl[(isothiazolyl)phenyl]indolocarbazole; phenyl(dicyanophenyl)indolocarbazole; phenyl(dicyanophenyl)indolocarbazole; bis(ethylphenyl)indolocarbazole; bis(isopropylphenyl)indolocarbazole; bis(tert-butylphenyl)indolocarbazole; phenyl[[phenyl(naphthalenyl)amino]phenyl]indolocarbazole; phenyl[[di(naphthalenyl)amino]phenyl]indolocarbazole; phenyl[[di(naphthalenyl)borane]phenyl]indolocarbazole; phenyl(terphenylyl)indolocarbazole; di(naphthalenyl)indolocarbazole; phenyl(anthracenyl)indolocarbazole; phenyl(tetracenyl)indolocarbazole; phenyl(perylenyl)indolocarbazole; phenyl(fluorenyl)indolocarbazole; phenyl[(n-butyl)phenyl]indolocarbazole; [(n-octyl)phenyl][(n-butyl)phenyl]indolocarbazole; phenyl[[phenyl(anthracenyl)amino]phenyl]indolocarbazole; phenyl[[di(anthracenyl)borane]phenyl]indolocarbazole; naphthalene(biphenylyl)indolocarbazole; phenyl[(phenylcarbazolyl)phenyl]indolocarbazole; phenyl[(carbazolyl)phenyl]indolocarbazole; phenyl[(dibenzothiophenyl)phenyl]indolocarbazole; phenyl[(phenylfenadinyl)phenyl]indolocarbazole; tetraphenylindolocarbazole; (dipyridine)(diphenyl)indolocarbazole; diphenyl(carbazolyl)indolocarbazole; phenyl(biphenylyl)indolocarbazole; tetraphenylindolocarbazole; di(naphthalenyl)indolocarbazole; phenyl[(phenyldibenzofuranyl)phenyl]indolocarbazole; phenyl(dibenzothiophenyl)indolocarbazole; phenyl(dibenzofuranyl)indolocarbazole; dimethyl(diphenyl)indolocarbazole; diphenyl[bis(diphenylamino)]indolocarbazole; diphenyl[bis(trimethylphenyl)boranyl]indolocarbazole; diphenyldicyanoindolocarbazole; dipyridinylindolocarbazole; bis(phenylcarbazolyl)indolocarbazole; bis[phenylindolocarbazolyl]benzene; bis[phenylindolocarbazolyl]benzene; bis[phenylindolocarbazolyl]biphenyl; bis[phenylindolocarbazolyl]naphthalene; bis[phenylindolocarbazolyl]anthracene; bis[phenylindolocarbazolyl]pyrene; bis[phenylindolocarbazolyl](cyano)benzene; (phenylindolocarbazolyl)[(carbazolyl)indolocarbazolyl]benzene; (phenylindolocarbazolyl)[(dibenzoselenophenyl)indolocarbazolyl]benzene; phenyl(phenylpyridinyl)indolocarbazole; phenyl(diphenylpyrimidinyl)indolocarbazole; phenyl(diphenyltriazinyl)indolocarbazole; phenyl(phenylpyrazinyl)indolocarbazole; phenyl(quinolinyl)indolocarbazole; phenyl(quinazolinyl)indolocarbazole; phenyl(phenylbenzimidazolyl)indolocarbazole; phenyl(cinnolinyl)indolocarbazole; phenyl(phenylcarbazolyl)indolocarbazole; methylbiphenyl(phenylpyridinyl)indolocarbazole; phenyl(carbazolylpyridinyl)indolocarbazole; phenyl[phenyl(carbazolyl)pyrimidinyl]indolocarbazole; phenyl[phenyl(biphenylyl)triazinyl]indolocarbazole; biphenyl(diphenyltriazinyl)indolocarbazole; phenyl[phenyl(biphenylyl)triazinyl]indolocarbazole; phenyl[di(biphenylyl)triazinyl]indolocarbazole; naphthalenyl(quinolinyl)indolocarbazole; phenyl(biphenylquinazolinyl)indolocarbazole; phenyl(biphenylindolyl)indolocarbazole; terphenyl(phenylpyridinyl)indolocarbazole; phenyl[di(biphenylyl)triazinyl]indolocarbazole; tetraphenyl(quinolinyl)indolocarbazole; phenyl(carbazolyl)(phenylcarbazolyl)indolocarbazole; diphenylaminophenyl(phenylquinazolinyl)indolocarbazole; phenyl{phenyl[(carbazolyl)methylphenyl]triazinyl}indolocarbazole; phenyl(naphthalenylcarbazolyl)indolocarbazole; phenyl(quinolinylcarbazolyl)indolocarbazole; phenyl(phenylpyridinyl)indolocarbazole; phenyl(phenylpyridinyl)indolocarbazole; biphenyl(phenylpyridinyl)indolocarbazole; terphenyl(phenylpyridinyl)indolocarbazole; phenyl(diphenylpyrimidinyl)indolocarbazole; (dibenzothiophenylphenyl)(diphenylpyrimidinyl)indolocarbazole; phenyl(diphenyltriazinyl)indolocarbazole; phenyl[phenyl(biphenylyl)triazinyl]indolocarbazole; biphenyl[phenyl(biphenylyl)triazinyl]indolocarbazole; phenyl[biphenyl(naphthalenyl)triazinyl]indolocarbazole; dibenzothiophenyl(diphenyltriazinyl)indolocarbazole; phenyl(quinolinyl)indolocarbazole; biphenyl(quinazolinyl)indolocarbazole; phenyl(quinoxalinyl)indolocarbazole; phenyl(phenylcarbazolyl)indolocarbazole; phenyl(phenylcarbazolyl)indolocarbazole; phenyl(phenylcarbazolyl)indolocarbazole; phenyl(phenylcarbazolyl)indolocarbazole; phenyl(dibenzothiophenylcarbazolyl)indolocarbazole; phenyl(phenylpyridoindolyl)indolocarbazole; phenyl(phenylpyridinyl)indolocarbazole; phenyl(phenylpyridinyl)indolocarbazole; phenyl(diphenylpyrimidinyl)indolocarbazole; phenyl(diphenyltriazinyl)indolocarbazole; naphthalene(diphenyltriazinyl)indolocarbazole; phenyl(diphenyltriazinyl)indolocarbazole; phenyl[di(cyanophenyl)triazinyl]indolocarbazole; phenyl(phenylbipyridinyl)indolocarbazole; tert-butylphenyl(quinolinyl)indolocarbazole; phenyl(phenylquinazolinyl)indolocarbazole; phenyl(quinoxalinyl)indolocarbazole; phenyl(carbazolepyridinyl)indolocarbazole; phenyl(phenylcarbazolylpyridinyl)indolocarbazole; phenyl(phenylcarbazolylpyridinyl)indolocarbazole; phenyl(triphenylenylpyridinyl)indolocarbazole; methylpyridinyl(triphenylenyl)indolocarbazole; phenylpyridinyl(phenanthrenyl)indolocarbazole; phenylpyridinyl(pyrenyl)indolocarbazole; phenylpyridinyl(terphenylyl)indolocarbazole; phenyl(phenylcarbazolyl)indolocarbazole; phenyl(phenylcarbazolyl)indolocarbazole; phenyl(phenylcarbazolyl)indolocarbazole; phenyl(phenylcarbazolyl)indolocarbazole; biphenyl(phenylcarbazolyl)indolocarbazole; phenyl(biphenylcarbazolyl)indolocarbazole; phenyl(phenylpyridoindolyl)indolocarbazole; phenyl(phenylpyridinyl)indolocarbazole; phenyl(diphenylpyrimidinyl)indolocarbazole; phenyl(diphenyltriazinyl)indolocarbazole; phenyl(quinolinyl)indolocarbazole; phenyl(phenylquinazolinyl)indolocarbazole; phenyl(phenylindolyl)indolocarbazole; phenyl(phenylbenzoindolyl)indolocarbazole; phenyl[phenyl(naphthalenyl)triazinyl]indolocarbazole; phenyl[phenyl(phenanthrenyl)triazinyl]indolocarbazole; phenyl[phenyl(fluorenyl)triazinyl]indolocarbazole; phenylpyridinyl(triphenylenyl)indolocarbazole; phenyl(phenylcarbazolyl)indolocarbazole; phenyl(phenylcarbazolyl)indolocarbazole; phenyl(phenylcarbazolyl)indolocarbazole; phenyl(phenylcarbazolyl)indolocarbazole; benzothiophenyl(phenylcarbazolyl)indolocarbazole; phenyl(naphthalenylcarbazolyl)indolocarbazole; phenyl(phenylazaoxaindenofluorenyl)indolocarbazole; phenyl(pyridinyl)indolocarbazole; phenyl(diphenylpyridinyl)indolocarbazole; phenyl(diphenylpyrimidinyl)indolocarbazole; phenyl[phenyl(dibenzofuranyl)pyrimidinyl]indolocarbazole; phenyl(diphenyltriazinyl)indolocarbazole; phenyl[phenyl(phenylanthracenyl)triazinyl]indolocarbazole; phenylpyridinyl(anthracenyl)indolocarbazole; phenyl(quinolinyl)indolocarbazole; phenyl(isoquinolinyl)indolocarbazole; phenyl(acridinyl)indolocarbazole; phenyl[(phenylanthracenyl)pyridinyl]indolocarbazole; phenyl(naphthalenylpyridinyl)indolocarbazole; phenyl(isoquinolinylpyridinyl)indolocarbazole; phenyl(phenylcarbazolyl)indolocarbazole; phenyl(phenylcarbazolyl)indolocarbazole; phenyl(phenylcarbazolyl)indolocarbazole; phenyl(phenylcarbazolyl)indolocarbazole; phenyl(methylphenylcarbazolyl)indolocarbazole; phenyl(terphenylcarbazolyl)indolocarbazole; bis[phenylindolocarbazolyl]pyridazine; bis[phenylindolocarbazolyl]pyridine; bis[phenylindolocarbazolyl]cyanopyridine; bis[phenylindolocarbazolyl]pyrazine; bis[phenylindolocarbazolyl]phenylpyrimidine; bis[phenylindolocarbazolyl]phenylcarbazole; tris[phenylindolocarbazolyl]triazine

### (Group 17A)

(diphenyl-d₁₀)[(biphenyldibenzothiophenyl)phenyl]triazine; (phenyl-d₅)(triphenylenylphenyl)dibenzothiophene; (dicarbazolyl-d₁₆)dibenzothiophened₆; [(dimethyl-d₆)fluorenyl][(diphenyltriazinyl)phenyl]dibenzothiophene

### (Group 18A)

phenyl(terphenylyl)indolocarbazole; (biphenylyl)(terphenylyl)indolocarbazole; (biphenylyl)(terphenylyl)indolocarbazole; bis(terphenylyl)indolocarbazole; bis(terphenylyl)indolocarbazole; bis(terphenylyl)indolocarbazole; bis(terphenylyl)indolocarbazole; bis(terphenylyl)indolocarbazole; (terphenylyl)(triphenylenyl)indolocarbazole; (terphenylyl)(dimethylfluorenyl)indolocarbazole; (terphenylyl)(spiro[cyclopentanefluorene]yl)indolocarbazole; (terphenylyl)(spiro[cyclohexanefluorene]yl)indolocarbazole; (terphenylyl)(dimethylfluorenyl)indolocarbazole; (terphenylyl)(spirobi[fluorene]yl)indolocarbazole; (terphenylyl)(dibenzofuranyl)indolocarbazole; (terphenylyl)(dibenzofuranyl)indolocarbazole; (terphenylyl)(dibenzofuranyl)indolocarbazole; (terphenylyl)(dibenzothiophenyl)indolocarbazole; (terphenylyl)(dibenzothiophenyl)indolocarbazole; (terphenylyl)(dibenzothiophenyl)indolocarbazole; (terphenylyl)(dibenzoselenophenyl)indolocarbazole; (terphenylyl)(dibenzoselenophenyl)indolocarbazole; (terphenylyl)dibenzoselenophene-indolocarbazole; (terphenylyl)phenylcarbazole-indolocarbazole; (terphenylyl)(carbazolylphenyl)indolocarbazole; (terphenylyl)(benzothienopyridinyl)indolocarbazole; (terphenylyl)(benzothienopyrimidinyl)indolocarbazole; (terphenylyl)(benzothienopyrimidinyl)indolocarbazole; (terphenylyl)phenylbenzothienopyrimidinyl)indolocarbazole; (terphenylyl)(diphenyltriazinyl)indolocarbazole; (terphenylyl)(phenylquinazolinyl)indolocarbazole; (terphenylyl)(dibenzoquinazolinyl)indolocarbazole; (terphenylyl)(dibenzoquinoxalinyl)indolocarbazole; [(phenyl-d₅)biphenylyl](biphenylyl)indolocarbazole; [(phenyl-d₅)biphenyl](phenyl-d₅)indolocarbazole; (terphenylyl)[(dimethyl-d₆)fluorene]indolocarbazole; (terphenylyl)(dibenzofuranylphenyl)indolocarbazole; (terphenylyl)(dibenzothiophenylphenyl)indolocarbazole; (terphenylyl)(triphenylenylphenyl)indolocarbazole; (terphenylyl)[(diphenyltriazinyl)phenyl]-indolocarbazole; (terphenylyl)(phenyl)indolocarbazole; (terphenylyl)(biphenylyl)indolocarbazole; (terphenylyl)(biphenylyl)indolocarbazole; di(terphenylyl)indolocarbazole; di(terphenylyl)indolocarbazole; (terphenylyl)(triphenylenyl)indolocarbazole; (terphenylyl)(dimethylfluorenyl)indolocarbazole; (terphenylyl)(spiro[cyclohexanefluorene]yl)indolocarbazole; (terphenylyl)(spiro[cyclopentanefluorene]yl)indolocarbazole; (terphenylyl)(spirobi[fluorene]yl)indolocarbazole; (terphenylyl)(dimethylfluorenyl)-indolocarbazole; (terphenylyl)(dibenzofuranyl)indolocarbazole; (terphenylyl)(dibenzofuranyl)indolocarbazole; (terphenylyl)(dibenzofuranyl)indolocarbazole; (terphenylyl)(dibenzothiophenyl)indolocarbazole; (terphenylyl)(dibenzothiophenyl)indolocarbazole; (terphenylyl)(dibenzothiophenyl)indolocarbazole; (terphenylyl)(dibenzoselenophenyl)indolocarbazole; (terphenylyl)(dibenzoselenophenyl)indolocarbazole; (terphenylyl)(dibenzoselenophenyl)indolocarbazole; (terphenylyl)(phenylcarbazolyl)indolocarbazole; (terphenylyl)[(carbazolyl)phenyl]indolocarbazole; (terphenylyl)(benzothienopyridinyl)indolocarbazole; (terphenylyl)(benzothienopyrimidinyl)indolocarbazole; (terphenylyl)(benzothienopyrimidinyl)indolocarbazole; (terphenylyl)(phenylbenzothienopyrimidinyl)indolocarbazole; (terphenylyl)(diphenylbenzothienopyrimidinyl)indolocarbazole; (terphenylyl)(diphenylbenzothienopyrimidinyl)indolocarbazole; (terphenylyl)(diphenyltriazinyl)indolocarbazole; (terphenylyl)(phenylquinazolinyl)indolocarbazole; (terphenylyl)(dibenzoquinazolinyl)indolocarbazole; (terphenylyl)(dibenzoquinoxalinyl)indolocarbazole; (terphenylyl)[(dimethyl-d₆)fluorenyl]indolocarbazole; (terphenylyl)[(diphenyltriazinyl)phenyl]indolocarbazole; [(phenyl-d₅)biphenylyl](biphenylyl)indolocarbazole; [(phenyl-d₅)biphenylyl](phenyl-d₅)indolocarbazole; (terphenylyl)(phenyl)indolocarbazole; (terphenylyl)(biphenylyl)indolocarbazole; (terphenylyl)(biphenylyl)indolocarbazole; di(terphenylyl)indolocarbazole; di(terphenylyl)indolocarbazole; (terphenylyl)(triphenylenyl)indolocarbazole; (terphenylyl)(dimethylfluorenyl)indolocarbazole; (terphenylyl)(spirobi[fluorene]yl)-indolocarbazole; (terphenylyl)(spiro[cyclopentanefluorene]yl)indolocarbazole; (terphenylyl)(dibenzofuranyl)indolocarbazole; (terphenylyl)(dibenzofuranyl)indolocarbazole; (terphenylyl)(dibenzofuranyl)indolocarbazole; (terphenylyl)(dibenzothiophenyl)indolocarbazole; (terphenylyl)(dibenzothiophenyl)indolocarbazole; (terphenylyl)(dibenzothiophenyl)indolocarbazole; (terphenylyl)(diphenyltriazinyl)indolocarbazole; (terphenylyl)(phenylquinazolinyl)indolocarbazole; (terphenylyl)(benzothienopyrimidinyl)indolocarbazole; (terphenylyl)(phenyl)indolocarbazole; (terphenylyl)(biphenylyl)indolocarbazole; (terphenylyl)(biphenylyl)indolocarbazole; di(terphenylyl)indolocarbazole; di(terphenylyl)indolocarbazole; (terphenylyl)(triphenylenyl)indolocarbazole; (terphenylyl)(dimethylfluorenyl)indolocarbazole; (terphenylyl)(spirobi[fluorene]yl)-indolocarbazole; (terphenylyl)(spiro[cyclopentanefluorene]yl)indolocarbazole; (terphenylyl)(dibenzofuranyl)indolocarbazole; (terphenylyl)(dibenzofuranyl)indolocarbazole; (terphenylyl)(dibenzofuranyl)indolocarbazole; (terphenylyl)(dibenzothiophenyl)indolocarbazole; (terphenylyl)(dibenzothiophenyl)indolocarbazole; (terphenylyl)(dibenzothiophenyl)indolocarbazole; (terphenylyl)(diphenyltriazinyl)indolocarbazole; (terphenylyl)(phenylquinazolinyl)indolocarbazole; (terphenylyl)(benzothienopyrimidinyl)indolocarbazole; (terphenylyl)(phenyl)indolocarbazole; (terphenylyl)(biphenylyl)indolocarbazole; (terphenylyl)(biphenylyl)indolocarbazole; di(terphenylyl)indolocarbazole; di(terphenylyl)indolocarbazole; (terphenylyl)(dimethylfluorenyl)indolocarbazole; (terphenylyl)(dibenzofuranyl)indolocarbazole; (terphenylyl)(dibenzothiophenyl)indolocarbazole; (terphenylyl)(dibenzothiophenyl)indolocarbazole; (terphenylyl)(benzothienopyridinyl)indolocarbazole; (terphenylyl)(benzothienopyrimidinyl)indolocarbazole; (terphenylyl)(diphenyltriazinyl)indolocarbazole; (terphenylyl)(phenylquinazolinyl)indolocarbazole; [(phenyl-d₅)biphenylyl](biphenylyl)indolocarbazole; [(phenyl-d₅)biphenylyl](phenyl-d₅)indolocarbazole; (terphenylyl)(phenyl)indolocarbazole; (terphenylyl)(biphenylyl)indolocarbazole; (terphenylyl)(biphenylyl)indolocarbazole; (terphenylyl)(dibenzothiophenyl)indolocarbazole; (terphenylyl)(dibenzothiophenyl)indolocarbazole; dibenzofuranyl(diphenyltriazinyl)biphenyl; dibenzothiophenyl(diphenyltriazinyl)biphenyl; dibenzoselenophenyl(diphenyltriazinyl)biphenyl; dibenzofuranyl(diphenyltriazinyl)terbenzene; dibenzothiophenyl(diphenyltriazinyl)terbenzene; dibenzoselenophenyl(diphenyltriazinyl)terbenzene; dibenzofuranyl(diphenyltriazinyl)terbenzene; dibenzothiophenyl(diphenyltriazinyl)terbenzene; dibenzoselenophenyl(diphenyltriazinyl)terbenzene; (phenyldibenzofuranyl)(diphenyltriazinyl)biphenyl; (phenyldibenzothiophenyl)(diphenyltriazinyl)biphenyl; (phenyldibenzoselenophenyl)(diphenyltriazinyl)biphenyl; [(biphenylyl)dibenzofuranyl](diphenyltriazinyl)biphenyl; [(biphenylyl)phenyldibenzothiophenyl](diphenyltriazinyl)biphenyl; [(biphenylyl)phenyldibenzoselenophenyl](diphenyltriazinyl)biphenyl; [(biphenylyl)dibenzofuranyl](diphenyltriazinyl)biphenyl; [(biphenylyl)phenyldibenzothiophenyl](diphenyltriazinyl)biphenyl; [(biphenylyl)phenyldibenzoselenophenyl](diphenyltriazinyl)biphenyl; (phenyldibenzofuranyl)(diphenyltriazinyl)terbenzene; (phenyldibenzothiophenyl)(diphenyltriazinyl)terbenzene; (phenyldibenzoselenophenyl)(diphenyltriazinyl)terbenzene; (phenyldibenzofuranyl)(diphenyltriazinyl)terbenzene; (phenyldibenzothiophenyl)(diphenyltriazinyl)terbenzene; (phenyldibenzoselenophenyl)(diphenyltriazinyl)terbenzene; [biphenylyl(dibenzofuranyl)](diphenyltriazinyl)terbenzene; [biphenylyl(dibenzothiophenyl)](diphenyltriazinyl)terbenzene; [biphenylyl(dibenzoselenophenyl)](diphenyltriazinyl)terbenzene; dibenzofuranyl[phenyl(biphenylyl)triazinyl]biphenyl; dibenzothiophenyl[phenyl(biphenylyl)triazinyl]biphenyl; dibenzoselenophenyl[phenyl(biphenylyl)triazinyl]biphenyl; dibenzofuranyl[phenyl(biphenylyl)triazinyl]biphenyl; dibenzothiophenyl[phenyl(biphenylyl)triazinyl]biphenyl; dibenzoselenophenyl[phenyl(biphenylyl)triazinyl]biphenyl; dibenzofuranyl[bis(biphenylyl)triazinyl]biphenyl; dibenzothiophenyl[bis(biphenylyl)triazinyl]biphenyl; dibenzoselenophenyl[bis(biphenylyl)triazinyl]biphenyl; dibenzofuranyl[dimethylfluorenyl(biphenylyl)triazinyl]biphenyl; dibenzothiophenyl[dimethylfluorenyl(biphenylyl)triazinyl]biphenyl; dibenzoselenophenyl[dimethylfluorenyl(biphenylyl)triazinyl]biphenyl; phenyldibenzofuranyl[phenyl(biphenylyl)triazinyl]biphenyl; phenyldibenzothiophenyl[phenyl(biphenylyl)triazinyl]biphenyl; phenyldibenzoselenophenyl[phenyl(biphenylyl)triazinyl]biphenyl; phenyldibenzofuranyl[phenyl(biphenylyl)triazinyl]biphenyl; phenyldibenzothiophenyl[phenyl(biphenylyl)triazinyl]biphenyl; phenyldibenzoselenophenyl[phenyl(biphenylyl)triazinyl]biphenyl; phenyldibenzofuranyl[dimethylfluorenyl(biphenylyl)triazinyl]biphenyl; phenyldibenzothiophenyl[dimethylfluorenyl(biphenylyl)triazinyl]biphenyl; phenyldibenzoselenophenyl[dimethylfluorenyl(biphenylyl)triazinyl]biphenyl; dibenzofuranyl[phenyl(biphenylyl)triazinyl]terbenzene; dibenzothiophenyl[phenyl(biphenylyl)triazinyl]terbenzene; dibenzoselenophenyl[phenyl(biphenylyl)triazinyl]terbenzene; dibenzofuranyl[phenyl(biphenylyl)triazinyl]terbenzene; dibenzothiophenyl[phenyl(biphenylyl)triazinyl]terbenzene; dibenzoselenophenyl[phenyl(biphenylyl)triazinyl]terbenzene; dibenzofuranyl[dimethylfluorenyl(biphenylyl)triazinyl]terbenzene; dibenzothiophenyl[dimethylfluorenyl(biphenylyl)triazinyl]terbenzene; dibenzoselenophenyl[dimethylfluorenyl(biphenylyl)triazinyl]terbenzene; (biphenylyl)dibenzofuranyl[phenyl(biphenylyl)triazinyl]biphenyl; (biphenylyl)dibenzothiophenyl[phenyl(biphenylyl)triazinyl]biphenyl; (biphenylyl)dibenzoselenophenyl[phenyl(biphenylyl)triazinyl]biphenyl; (biphenylyl)dibenzofuranyl[phenyl(biphenylyl)triazinyl]biphenyl; (biphenylyl)dibenzothiophenyl[phenyl(biphenylyl)triazinyl]biphenyl; (biphenylyl)dibenzoselenophenyl[phenyl(biphenylyl)triazinyl]biphenyl; (biphenylyl)dibenzofuranyl[dimethylfluorenyl(biphenylyl)triazinyl]biphenyl; (biphenylyl)dibenzothiophenyl[dimethylfluorenyl(biphenylyl)triazinyl]biphenyl; (biphenylyl)dibenzoselenophenyl[dimethylfluorenyl(biphenylyl)triazinyl]biphenyl; diphenyltriazinyl(dibenzofuranyl)terbenzene; diphenyltriazinyl(dibenzothiophenyl)]terbenzene; diphenyltriazinyl(dibenzoselenophenyl)terbenzene; dibenzofuranyl(diphenyltriazinyl)terbenzene; dibenzothiophenyl(diphenyltriazinyl)terbenzene; dibenzoselenophenyl(diphenyltriazinyl)terbenzene; dibenzofuranyl(diphenyltriazinyl)biphenyl; dibenzothiophenyl(diphenyltriazinyl)biphenyl; dibenzoselenophenyl(diphenyltriazinyl)biphenyl; dibenzofuranyl(diphenyltriazinyl)biphenyl; dibenzothiophenyl(diphenyltriazinyl)biphenyl; dibenzoselenophenyl(diphenyltriazinyl)biphenyl; dibenzofuranyl(diphenyltriazinyl)biphenyl; dibenzothiophenyl(diphenyltriazinyl)biphenyl; dibenzoselenophenyl(diphenyltriazinyl)biphenyl; dibenzofuranyl(diphenyltriazinyl)terbenzene; dibenzothiophenyl(diphenyltriazinyl)terbenzene; dibenzoselenophenyl(diphenyltriazinyl)terbenzene; dibenzofuranyl(diphenyltriazinyl)terbenzene; dibenzothiophenyl(diphenyltriazinyl)terbenzene; dibenzoselenophenyl(diphenyltriazinyl)terbenzene; dibenzofuranyl(diphenyltriazinyl)terbenzene; dibenzothiophenyl(diphenyltriazinyl)terbenzene; dibenzoselenophenyl(diphenyltriazinyl)terbenzene; diphenyldibenzofuranyl(diphenyltriazinyl)biphenyl; diphenyldibenzothiophenyl(diphenyltriazinyl)biphenyl; diphenyldibenzoselenophenyl(diphenyltriazinyl)biphenyl; diphenyldibenzofuranyl(diphenyltriazinyl)biphenyl; diphenyldibenzothiophenyl(diphenyltriazinyl)biphenyl; diphenyldibenzoselenophenyl(diphenyltriazinyl)biphenyl; phenyldibenzofuranyl(diphenyltriazinyl)biphenyl; phenyldibenzothiophenyl(diphenyltriazinyl)biphenyl; phenyldibenzoselenophenyl(diphenyltriazinyl)biphenyl; (phenyldibenzofuranyl)(diphenyltriazinyl)(pyridinyl)benzene; (phenyldibenzothiophenyl)(diphenyltriazinyl)(pyridinyl)benzene; (phenyldibenzoselenophenyl)(diphenyltriazinyl)(pyridinyl)benzene; (phenyldibenzofuranyl)(diphenyltriazinyl)(pyridinyl)benzene; (phenyldibenzothiophenyl)(diphenyltriazinyl)(pyridinyl)benzene; (phenyldibenzoselenophenyl)(diphenyltriazinyl)(pyridinyl)benzene; [(pyridinyl)dibenzofuranyl](diphenyltriazinyl)biphenyl; [(pyridinyl)dibenzothiophenyl](diphenyltriazinyl)biphenyl; [(pyridinyl)phenyldibenzoselenophenyl](diphenyltriazinyl)biphenyl; diphenyltriazinyl(dibenzofuranyl)terbenzene; diphenyltriazinyl(dibenzothiophenyl)terbenzene; diphenyltriazinyl(dibenzoselenophenyl)terbenzene; dibenzofuranyl(diphenyltriazinyl)terbenzene; dibenzothiophenyl(diphenyltriazinyl)terbenzene; dibenzoselenophenyl(diphenyltriazinyl)terbenzene; (dibenzofuranyl)(dimethylfluorenyl)(diphenyltriazinyl)benzene; (dibenzothiophenyl)(dimethylfluorenyl)(diphenyltriazinyl)benzene; (dibenzoselenophenyl)(dimethylfluorenyl)(diphenyltriazinyl)benzene; diphenyltriazinyl(dimethylfluorenyl)biphenyl; diphenyltriazinyl(spiro[cyclopentanefluorene]yl)biphenyl; diphenyltriazinyl(dimethylfluorenyl)terbenzene; diphenylfluorenyl(diphenyltriazinyl)biphenyl; dimethylfluorenyl[phenyl(biphenylyl)triazinyl]biphenyl; dimethylfluorenyl[phenyl(biphenylyl)triazinyl]biphenyl; dimethylfluorenyl[phenyl(dimethylfluorenyl)triazinyl]terbenzene; diphenyl[bis(dimethylfluorenyl)phenyl]triazine; phenyl(diphenyltriazinyl)dibenzofuran; phenyl(diphenyltriazinyl)dibenzothiophene; phenyl(diphenyltriazinyl)dibenzoselenophene; phenyl[phenyl(biphenylyl)triazinyl]dibenzofuran; phenyl[phenyl(biphenylyl)triazinyl]dibenzothiophene; phenyl[phenyl(biphenylyl)triazinyl]dibenzoselenophene; phenyl[phenyl(biphenylyl)triazinyl]dibenzofuran; phenyl[phenyl(biphenylyl)triazinyl]dibenzothiophene; phenyl[phenyl(biphenylyl)triazinyl]dibenzoselenophene; phenyl[bis(biphenylyl)triazinyl]dibenzofuran; phenyl[bis(biphenylyl)triazinyl]dibenzothiophene; phenyl[bis(biphenylyl)triazinyl]benzselenophene; phenyl[bis(biphenylyl)triazinyl]dibenzofuran; phenyl[bis(biphenylyl)triazinyl]dibenzothiophene; phenyl[bis(biphenylyl)triazinyl]benzselenophene; phenyl[bis(biphenylyl)triazinyl]dibenzofuran; phenyl[bis(biphenylyl)triazinyl]dibenzothiophene; phenyl[bis(biphenylyl)triazinyl]benzselenophene; phenyl[(diphenyltriazinyl)phenyl]dibenzofuran; phenyl[(diphenyltriazinyl)phenyl]dibenzothiophene; phenyl[(diphenyltriazinyl)phenyl]benzselenophene; phenyl{[phenyl(biphenylyl)triazinyl]phenyl}dibenzofuran; phenyl{[phenyl(biphenylyl)triazinyl]phenyl}dibenzothiophene; phenyl{[phenyl(biphenylyl)triazinyl]phenyl}benzselenophene; phenyl{[phenyl(biphenylyl)triazinyl]phenyl}dibenzofuran; phenyl{[phenyl(biphenylyl)triazinyl]phenyl}dibenzothiophene; phenyl{[phenyl(biphenylyl)triazinyl]phenyl}benzselenophene; phenyl[(diphenyltriazinyl)phenyl]dibenzofuran; phenyl[(diphenyltriazinyl)phenyl]dibenzothiophene; phenyl[(diphenyltriazinyl)phenyl]benzselenophene; phenyl{[phenyl(biphenylyl)triazinyl]phenyl}dibenzofuran; phenyl{[phenyl(biphenylyl)triazinyl]phenyl}dibenzothiophene; phenyl{[phenyl(biphenylyl)triazinyl]phenyl}benzselenophene; phenyl{[phenyl(biphenylyl)triazinyl]phenyl}dibenzofuran; phenyl{[phenyl(biphenylyl)triazinyl]phenyl}dibenzothiophene; phenyl{[phenyl(biphenylyl)triazinyl]phenyl}benzselenophene; phenyldibenzofuran(diphenyltriazinyl)biphenyl; phenyldibenzothiophene(diphenyltriazinyl)biphenyl; phenylbenzselenophene(diphenyltriazinyl)biphenyl; phenyldibenzofuran[phenyl(biphenylyl)triazinyl]biphenyl; phenyldibenzothiophene[phenyl(biphenylyl)triazinyl]biphenyl; phenylbenzselenophene[phenyl(biphenylyl)triazinyl]biphenyl; phenyldibenzofuran[phenyl(biphenylyl)triazinyl]biphenyl; phenyldibenzothiophene[phenyl(biphenylyl)triazinyl]biphenyl; phenylbenzselenophene[phenyl(biphenylyl)triazinyl]biphenyl; phenyldibenzofuran(diphenyltriazinyl)biphenyl; phenyldibenzothiophene(diphenyltriazinyl)biphenyl; phenylbenzselenophene(diphenyltriazinyl)biphenyl; phenyldibenzofuran[phenyl(biphenylyl)triazinyl]biphenyl; phenyldibenzothiophene[phenyl(biphenylyl)triazinyl]biphenyl; phenylbenzselenophene[phenyl(biphenylyl)triazinyl]biphenyl; phenyldibenzofuran[phenyl(biphenylyl)triazinyl]biphenyl; phenyldibenzothiophene[phenyl(biphenylyl)triazinyl]biphenyl; phenylbenzselenophene[phenyl(biphenylyl)triazinyl]biphenyl; phenyldibenzofuran(diphenyltriazinyl)biphenyl; phenyldibenzothiophene(diphenyltriazinyl)biphenyl; phenylbenzselenophene(diphenyltriazinyl)biphenyl; phenyldibenzofuran[phenyl(biphenylyl)triazinyl]biphenyl; phenyldibenzothiophene[phenyl(biphenylyl)triazinyl]biphenyl; phenylbenzselenophene[phenyl(biphenylyl)triazinyl]biphenyl; phenyldibenzofuran[phenyl(biphenylyl)triazinyl]biphenyl; phenyldibenzothiophene[phenyl(biphenylyl)triazinyl]biphenyl; phenylbenzselenophene[phenyl(biphenylyl)triazinyl]biphenyl; biphenylyl(diphenyltriazinyl)dibenzofuran; biphenylyl(diphenyltriazinyl)dibenzothiophene; biphenylyl(diphenyltriazinyl)benzselenophene; biphenylyl[phenyl(biphenylyl)triazinyl]dibenzofuran; biphenylyl[phenyl(biphenylyl)triazinyl]dibenzothiophene; biphenylyl[phenyl(biphenylyl)triazinyl]benzselenophene; biphenylyl[phenyl(biphenylyl)triazinyl]dibenzofuran; biphenylyl[phenyl(biphenylyl)triazinyl]dibenzothiophene; biphenylyl[phenyl(biphenylyl)triazinyl]benzselenophene; biphenylyl[(diphenyltriazinyl)phenyl]dibenzofuran; biphenylyl[(diphenyltriazinyl)phenyl]dibenzothiophene; biphenylyl[(diphenyltriazinyl)phenyl]benzselenophene; [(biphenylyl)dibenzofuranyl][phenyl(biphenylyl)triazinyl]benzene; [(biphenylyl)dibenzothiophenyl][phenyl(biphenylyl)triazinyl]benzene; [(biphenylyl)benzselenophenyl][phenyl(biphenylyl)triazinyl]benzene; [(biphenylyl)dibenzofuranyl][phenyl(biphenylyl)triazinyl]benzene; [(biphenylyl)dibenzothiophenyl][phenyl(biphenylyl)triazinyl]benzene; [(biphenylyl)benzselenophenyl][phenyl(biphenylyl)triazinyl]benzene; biphenylyl[(diphenyltriazinyl)phenyl]dibenzofuran; biphenylyl[(diphenyltriazinyl)phenyl]dibenzothiophene; biphenylyl[(diphenyltriazinyl)phenyl]benzselenophene; [(biphenylyl)dibenzofuranyl][phenyl(biphenylyl)triazinyl]benzene; [(biphenylyl)dibenzothiophenyl][phenyl(biphenylyl)triazinyl]benzene; [(biphenylyl)benzselenophenyl][phenyl(biphenylyl)triazinyl]benzene; [(biphenylyl)dibenzofuranyl][phenyl(biphenylyl)triazinyl]benzene; [(biphenylyl)dibenzothiophenyl][phenyl(biphenylyl)triazinyl]benzene; [(biphenylyl)benzselenophenyl][phenyl(biphenylyl)triazinyl]benzene; biphenylyl(diphenyltriazinyl)dibenzofuran; biphenylyl(diphenyltriazinyl)dibenzothiophene; biphenylyl(diphenyltriazinyl)benzselenophene; biphenylyl[phenyl(biphenylyl)triazinyl]dibenzofuran; biphenylyl[phenyl(biphenylyl)triazinyl]dibenzothiophene; biphenylyl[phenyl(biphenylyl)triazinyl]benzselenophene; biphenylyl[phenyl(biphenylyl)triazinyl]dibenzofuran; biphenylyl[phenyl(biphenylyl)triazinyl]dibenzothiophene; biphenylyl[phenyl(biphenylyl)triazinyl]benzselenophene; biphenylyl[(diphenyltriazinyl)phenyl]dibenzofuran; biphenylyl[(diphenyltriazinyl)phenyl]dibenzothiophene; biphenylyl[(diphenyltriazinyl)phenyl]benzselenophene; [(biphenylyl)dibenzofuranyl][phenyl(biphenylyl)triazinyl]benzene; [(biphenylyl)dibenzothiophenyl][phenyl(biphenylyl)triazinyl]benzene; [(biphenylyl)benzselenophenyl][phenyl(biphenylyl)triazinyl]benzene; [(biphenylyl)dibenzofuranyl][phenyl(biphenylyl)triazinyl]benzene; [(biphenylyl)dibenzothiophenyl][phenyl(biphenylyl)triazinyl]benzene; [(biphenylyl)benzselenophenyl][phenyl(biphenylyl)triazinyl]benzene; biphenylyl[(diphenyltriazinyl)phenyl]dibenzofuran; biphenylyl[(diphenyltriazinyl)phenyl]dibenzothiophene; biphenylyl[(diphenyltriazinyl)phenyl]benzselenophene; [(biphenylyl)dibenzofuranyl][phenyl(biphenylyl)triazinyl]benzene; [(biphenylyl)dibenzothiophenyl][phenyl(biphenylyl)triazinyl]benzene; [(biphenylyl)benzselenophenyl][phenyl(biphenylyl)triazinyl]benzene; [(biphenylyl)dibenzofuranyl][phenyl(biphenylyl)triazinyl]benzene; [(biphenylyl)dibenzothiophenyl][phenyl(biphenylyl)triazinyl]benzene; [(biphenylyl)benzselenophenyl][phenyl(biphenylyl)triazinyl]benzene; phenyl[phenyl(dimethylfluorenyl)triazinyl]dibenzofuran; phenyl[phenyl(dimethylfluorenyl)triazinyl]dibenzothiophene; phenyl[phenyl(dimethylfluorenyl)triazinyl]benzselenophene; phenyl[biphenylyl(dimethylfluorenyl)triazinyl]dibenzofuran; phenyl[biphenylyl(dimethylfluorenyl)triazinyl]dibenzothiophene; phenyl[biphenylyl(dimethylfluorenyl)triazinyl]benzselenophene; phenyl[biphenylyl(dimethylfluorenyl)triazinyl]dibenzofuran; phenyl[biphenylyl(dimethylfluorenyl)triazinyl]dibenzothiophene; phenyl[biphenylyl(dimethylfluorenyl)triazinyl]benzselenophene; phenyldibenzofuranyl[phenyl(dimethylfluorenyl)triazinyl]benzene; phenyldibenzothiophenyl[phenyl(dimethylfluorenyl)triazinyl]benzene; phenylbenzselenophenyl[phenyl(dimethylfluorenyl)triazinyl]benzene; phenyldibenzofuranyl[bis(dimethylfluorenyl)triazinyl]benzene; phenyldibenzothiophenyl[bis(dimethylfluorenyl)triazinyl]benzene; phenylbenzselenophenyl[bis(dimethylfluorenyl)triazinyl]benzene; biphenylyl[phenyl(dimethylfluorenyl)triazinyl]dibenzofuran; biphenylyl[phenyl(dimethylfluorenyl)triazinyl]dibenzothiophene; biphenylyl[phenyl(dimethylfluorenyl)triazinyl]benzselenophene; biphenylyl[phenyl(dimethylfluorenyl)triazinyl]dibenzofuran; biphenylyl[phenyl(dimethylfluorenyl)triazinyl]dibenzothiophene; biphenylyl[phenyl(dimethylfluorenyl)triazinyl]benzselenophene; [(biphenylyl)dibenzofuranyl][phenyl(dimethylfluorenyl)triazinyl]benzene; [(biphenylyl)dibenzothiophenyl][phenyl(dimethylfluorenyl)triazinyl]benzene; [(biphenylyl)benzselenophenyl][phenyl(dimethylfluorenyl)triazinyl]benzene; [(biphenylyl)dibenzofuranyl][phenyl(dimethylfluorenyl)triazinyl]benzene; [(biphenylyl)dibenzothiophenyl][phenyl(dimethylfluorenyl)triazinyl]benzene; [(biphenylyl)benzselenophenyl][phenyl(dimethylfluorenyl)triazinyl]benzene; phenyl[phenyl(dimethylfluorenylphenyl)triazinyl]dibenzofuran; phenyl[phenyl(dimethylfluorenylphenyl)triazinyl]dibenzothiophene; phenyl[phenyl(dimethylfluorenylphenyl)triazinyl]benzselenophene; phenyl[phenyl(dimethylfluorenyl)triazinyl]dibenzofuran; phenyl[phenyl(dimethylfluorenyl)triazinyl]dibenzothiophene; phenyl[phenyl(dimethylfluorenyl)triazinyl]benzselenophene; phenyl[(biphenylyl)dibenzofuranyl](dimethylfluorenyl)triazine; phenyl[(biphenylyl)dibenzothiophenyl](dimethylfluorenyl)triazine; phenyl[(biphenylyl)benzselenophenyl](dimethylfluorenyl)triazine; [(biphenylyl)dibenzofuranyl][phenyl(dimethylfluorenyl)triazinyl]benzene; [(biphenylyl)dibenzothiophenyl][phenyl(dimethylfluorenyl)triazinyl]benzene; [(biphenylyl)benzselenophenyl][phenyl(dimethylfluorenyl)triazinyl]benzene; phenyl(phenyldibenzofuranyl)(phenyldimethylfluorenyl)triazine; phenyl(phenyldibenzothiophenyl)(phenyldimethylfluorenyl)triazine; phenyl(phenylbenzselenophenyl)(phenyldimethylfluorenyl)triazine; phenyl(phenyldibenzofuranyl)(dimethylfluorenyl)triazine; phenyl(phenyldibenzothiophenyl)(dimethylfluorenyl)triazine; phenyl(phenylbenzselenophenyl)(dimethylfluorenyl)triazine; diphenyl(phenyldibenzofuranyl)triazine; diphenyl(phenyldibenzothiophenyl)triazine; diphenyl(phenylbenzselenophenyl)triazine; diphenyltriazinyl(phenyldibenzofuranyl)benzene; diphenyltriazinyl(phenyldibenzothiophenyl)benzene; diphenyltriazinyl(phenylbenzselenophenyl)benzene; diphenyltriazinyl[(biphenylyl)dibenzofuranyl]benzene; diphenyltriaziny|[(biphenylyl)dibenzothiophenyl]benzene; diphenyltriazinyl[(biphenylyl)benzselenophenyl]benzene; diphenyltriazinyl(diphenyldibenzofuranyl)benzene; diphenyltriazinyl(diphenyldibenzothiophenyl)benzene; diphenyltriazinyl(diphenylbenzselenophenyl)benzene; diphenyl(phenyldibenzofuranyl)triazine; diphenyl(phenyldibenzothiophenyl)triazine; diphenyl(phenylbenzselenophenyl)triazine; diphenyl[(biphenylyl)dibenzofuranyl]triazine; diphenyl[(biphenylyl)dibenzothiophenyl]triazine; diphenyl[(biphenylyl)benzselenophenyl]triazine; diphenyl[(biphenylyl)dibenzofuranyl]triazine; diphenyl[(biphenylyl)dibenzothiophenyl]triazine; diphenyl[(biphenylyl)benzselenophenyl]triazine; diphenyltriazinyl(diphenyldibenzofuranyl)benzene; diphenyltriazinyl(diphenyldibenzothiophenyl)benzene; diphenyltriazinyl(diphenylbenzselenophenyl)benzene; diphenyltriazinyl(biphenylyldibenzofuranyl)benzene; diphenyltriazinyl(biphenylyldibenzothiophenyl)benzene; diphenyltriazinyl(biphenylylbenzselenophenyl)benzene; diphenyl[phenyldibenzofuranyl]triazine; diphenyl[phenyldibenzothiophenyl]triazine; diphenyl[phenylbenzselenophenyl]triazine; diphenyltriazinyl(phenyldibenzofuranyl)benzene; diphenyltriazinyl(phenyldibenzothiophenyl)benzene; diphenyltriazinyl(phenylbenzselenophenyl)benzene; diphenyltriazinyl(phenyldibenzofuranyl)benzene; diphenyltriazinyl(phenyldibenzothiophenyl)benzene; diphenyltriazinyl(phenylbenzselenophenyl)benzene; diphenyltriazinyl(biphenylyldibenzofuranyl)benzene; diphenyltriazinyl(biphenylyldibenzothiophenyl)benzene; diphenyltriazinyl(biphenylylbenzselenophenyl)benzene; diphenyltriazinyl(biphenylyldibenzofuranyl)benzene; diphenyltriazinyl(biphenylyldibenzothiophenyl)benzene; diphenyltriazinyl(biphenylylbenzselenophenyl)benzene; diphenyltriazinyl(phenyldibenzofuranyl)benzene; diphenyltriazinyl(phenyldibenzothiophenyl)benzene; diphenyltriazinyl(phenylbenzselenophenyl)benzene; diphenyltriazinyl(phenyldibenzofuranyl)pyridine; diphenyltriazinyl(phenyldibenzothiophenyl)pyridine; diphenyltriazinyl(phenylbenzselenophenyl)pyridine; phenyl(phenylpyridinyl)triazinyl(phenyldibenzofuranyl)pyridine; phenyl(phenylpyridinyl)triazinyl(phenyldibenzothiophenyl)pyridine; phenyl(phenylpyridinyl)triazinyl(phenylbenzselenophenyl)pyridine; phenyl(phenylpyridinyl)triazinyl(phenyldibenzofuranyl)benzene; phenyl(phenylpyridinyl)triazinyl(phenyldibenzothiophenyl)benzene; phenyl(phenylpyridinyl)triazinyl(phenylbenzselenophenyl)benzene; diphenyltriazinyl(biphenylyldibenzofuranyl)pyridine; diphenyltriazinyl(biphenylyldibenzothiophenyl)pyridine; diphenyltriazinyl(biphenylylbenzselenophenyl)pyridine; phenyl(dimethylindenopyridinyl)(phenyldibenzofuranyl)triazine; phenyl(dimethylindenopyridinyl)(phenyldibenzothiophenyl)triazine; phenyl(dimethylindenopyridinyl)(phenylbenzselenophenyl)triazine; (biphenylyl)(dimethylindenopyridinyl)(phenyldibenzofuranyl)triazine; (biphenylyl)(dimethylindenopyridinyl)(phenyldibenzothiophenyl)triazine; (biphenylyl)(dimethylindenopyridinyl)(phenylbenzselenophenyl)triazine; phenyl(dimethylindenopyridinyl)(phenyldibenzofuranyl)triazine; phenyl(dimethylindenopyridinyl)(phenyldibenzothiophenyl)triazine; phenyl(dimethylindenopyridinyl)(phenylbenzselenophenyl)triazine; phenyl(spiro[cyclohexaneindenopyridine]yl)(phenyldibenzofuranyl)triazine; phenyl(spiro[cyclohexaneindenopyridine]yl)(phenyldibenzothiophenyl)triazine; phenyl(spiro[cyclohexaneindenopyridine]yl)(phenylbenzselenophenyl)triazine; phenyl(spirobi[fluorene]yl)(phenyldibenzofuranyl)triazine; phenyl(spirobi[fluorene]yl)(phenyldibenzothiophenyl)triazine; phenyl(spirobi[fluorene]yl)(phenylbenzselenophenyl)triazine; benzene-d₃[(diphenyl-d₁₀)triazinyl][(phenyl-d₅)dibenzofuranyl-d₆]; benzene-d₃[(diphenyl-d₁₀)triazinyl][(phenyl-d₅)dibenzothiophenyl-d₆]; benzene-d₃[(diphenyl-d₁₀)triazinyl][(phenyl-d₅)benzselenophenyl-d₆]; benzene-d₃[(diphenyl-d₁₀)triazinyl][(biphenylyl-d₅)dibenzofuranyl-d₆]; benzene-d₃[(diphenyl-d₁₀)triazinyl][(biphenylyl-d₈)dibenzothiophenyl-d₆]; benzene-d₃[(diphenyl-d₁₀)triazinyl][(biphenylyl-d₅)benzselenophenyl-d₆]; phenyl[(dimethyl-d₆)fluorene][phenyldibenzofuranyl]triazine; phenyl[(dimethyl-d₆)fluorene][phenyldibenzothiophenyl]triazine; phenyl[(dimethyl-d₆)fluorene][phenylbenzselenophenyl]triazine; benzene-d₃[(diphenyl-d₁₀)triazinyl][(biphenylyl-d₈)dibenzofuranyl-d₆]; benzene-d₃[(diphenyl-d₁₀)triazinyl][(biphenylyl-d₈)dibenzothiophenyl-d₆]; benzene-d₃[(diphenyl-d₁₀)triazinyl][(biphenylyl-da)benzselenophenyl-d₆]; [(diphenyl-d₁₀)triazinyl][(biphenylyl)dibenzofuranyl]benzene; [(diphenyl-d₁₀)triazinyl][(biphenylyl)dibenzothiophenyl]benzene; [(diphenyl-d₁₀)triazinyl][(biphenylyl)benzselenophenyl]benzene; (diphenyltriazinyl)[(biphenylyl)dibenzofuranyl]biphenyl; (diphenyltriazinyl)[(biphenylyl)dibenzothiophenyl]biphenyl; (diphenyltriazinyl)[(biphenylyl)benzselenophenyl]biphenyl; [bis(fluorophenyl)triazinyl][(biphenylyl)dibenzofuranyl]benzene; [bis(fluorophenyl)triazinyl][(biphenylyl)dibenzothiophenyl]benzene; [bis(fluorophenyl)triazinyl][(biphenylyl)benzselenophenyl]benzene; diphenyltriazinyl[(dimethylfluorenyl)dibenzofuranyl]benzene; diphenyltriazinyl[(dimethylfluorenyl)dibenzothiophenyl]benzene; diphenyltriazinyl[(dimethylfluorenyl)benzselenophenyl]benzene; diphenyltriazinyl[(dimethylfluorenyl)dibenzofuranyl]biphenyl; diphenyltriazinyl[(dimethylfluorenyl)dibenzothiophenyl]biphenyl; diphenyltriazinyl[(dimethylfluorenyl)benzselenophenyl]biphenyl; diphenyltriazinyl[(dimethylfluorenyl)dibenzofuranyl]biphenyl; diphenyltriazinyl[(dimethylfluorenyl)dibenzothiophenyl]biphenyl; diphenyltriazinyl[(dimethylfluorenyl)benzselenophenyl]biphenyl; diphenyltriazinyl[(dimethylfluorenyl)dibenzofuranyl]biphenyl; diphenyltriazinyl[(dimethylfluorenyl)dibenzothiophenyl]biphenyl; diphenyltriazinyl[(dimethylfluorenyl)benzselenophenyl]biphenyl; diphenyltriazinyl[(dimethylfluorenyl)dibenzofuranyl]pyridine; diphenyltriazinyl[(dimethylfluorenyl)dibenzothiophenyl]pyridine; diphenyltriazinyl[(dimethylfluorenyl)benzselenophenyl]pyridine; diphenyltriazinyl[(dimethylfluorenyl)dibenzofuranyl]benzene; diphenyltriazinyl[(dimethylfluorenyl)dibenzothiophenyl]benzene; diphenyltriazinyl[(dimethylfluorenyl)benzselenophenyl]benzene; diphenyltriazinyl[(dimethylfluorenyl)dibenzofuranyl]benzene; diphenyltriazinyl[(dimethylfluorenyl)dibenzothiophenyl]benzene; diphenyltriazinyl[(dimethylfluorenyl)benzselenophenyl]benzene; diphenyltriazinyl[(dimethylfluorenyl)dibenzofuranyl]benzene; diphenyltriazinyl[(dimethylfluorenyl)dibenzothiophenyl]benzene; diphenyltriazinyl[(dimethylfluorenyl)benzselenophenyl]benzene; diphenyltriazinyl[(dimethylfluorenyl)dibenzofuranyl]benzene; diphenyltriazinyl[(dimethylfluorenyl)dibenzothiophenyl]benzene; diphenyltriazinyl[(dimethylfluorenyl)benzselenophenyl]benzene; diphenyltriazinyl[(dimethylfluorenyl)dibenzofuranyl]biphenyl; diphenyltriazinyl[(dimethylfluorenyl)dibenzothiophenyl]biphenyl; diphenyltriazinyl[(dimethylfluorenyl)benzselenophenyl]biphenyl; [phenyl(biphenylyl)triazinyl][(dimethylfluorenyl)dibenzofuranyl]benzene; [phenyl(biphenylyl)triazinyl][(dimethylfluorenyl)dibenzothiophenyl]benzene; [phenyl(biphenylyl)triazinyl][(dimethylfluorenyl)benzselenophenyl]benzene; [phenyl(biphenylyl)triazinyl][(dimethylfluorenyl)dibenzofuranyl]benzene; [phenyl(biphenylyl)triazinyl][(dimethylfluorenyl)dibenzothiophenyl]benzene; [phenyl(biphenylyl)triazinyl][(dimethylfluorenyl)benzselenophenyl]benzene; [phenyl(dimethylfluorenyl)triazinyl][(dimethylfluorenyl)dibenzofuranyl]benzene; [phenyl(dimethylfluorenyl)triazinyl][(dimethylfluorenyl)dibenzothiophenyl]benzene; [phenyl(dimethylfluorenyl)triazinyl][(dimethylfluorenyl)benzselenophenyl]benzene; diphenyltriazinyl[(dimethylfluorenyl)dibenzofuranyl]benzene; diphenyltriazinyl[(dimethylfluorenyl)dibenzothiophenyl]benzene; diphenyltriazinyl[(dimethylfluorenyl)benzselenophenyl]benzene; diphenyltriazinyl[bis(dimethylfluorenyl)dibenzofuranyl]benzene; diphenyltriazinyl[bis(dimethylfluorenyl)dibenzothiophenyl]benzene; diphenyltriazinyl[bis(dimethylfluorenyl)benzselenophenyl]benzene; [phenyl(terphenylyl)triazinyl][(dimethylfluorenyl)dibenzofuranyl]benzene; [phenyl(terphenylyl)triazinyl]triazinyl[(dimethylfluorenyl)dibenzothiophenyl]benzene; [phenyl(terphenylyl)triazinyl][(dimethylfluorenyl)benzselenophenyl]benzene; diphenyltriazinyl[(spiro[cyclopentanefluorene]yl)dibenzofuranyl]benzene; diphenyltriazinyl[(spiro[cyclopentanefluorene]yl)dibenzothiophenyl]benzene; diphenyltriazinyl[(spiro[cyclopentanefluorene]yl)benzselenophenyl]benzene; diphenyltriazinyl[(diphenylfluorenyl)dibenzofuranyl]benzene; diphenyltriazinyl[(diphenylfluorenyl)dibenzothiophenyl]benzene; diphenyltriazinyl[(diphenylfluorenyl)benzselenophenyl]benzene; diphenyltriazinyl[(spirobi[fluorene]yl)dibenzofuranyl]benzene; diphenyltriazinyl[(spirobi[fluorene]yl)dibenzothiophenyl]benzene; diphenyltriazinyl[(spirobi[fluorene]yl)benzselenophenyl]benzene; diphenyltriazinyl[phenyl(dimethylfluorenyl)dibenzofuranyl]benzene; diphenyltriazinyl[phenyl(dimethylfluorenyl)dibenzothiophenyl]benzene; diphenyltriazinyl[phenyl(dimethylfluorenyl)benzselenophenyl]benzene; diphenyltriazinyl[phenyl(dimethylfluorenyl)dibenzofuranyl]benzene; diphenyltriazinyl[phenyl(dimethylfluorenyl)dibenzothiophenyl]benzene; diphenyltriazinyl[phenyl(dimethylfluorenyl)benzselenophenyl]benzene; [(diphenyltriazinyl)phenyl](phenyldimethylfluorenyl)dibenzofuran; [(diphenyltriazinyl)phenyl](phenyldimethylfluorenyl)dibenzothiophene; [(diphenyltriazinyl)phenyl](phenyldimethylfluorenyl)benzselenophene; diphenyltriazinyl{[(dimethyl-d₆)fluorenyl]dibenzofuranyl}benzene; diphenyltriazinyl{[(dimethyl-d₆)fluorenyl]dibenzothiophenyl}benzene; diphenyltriazinyl{[(dimethyl-d₆)fluorenyl]benzselenophenyl}benzene; diphenyltriazinyl[(dimethylfluorenyl)dibenzofuranyl]benzene; diphenyltriazinyl[(dimethylfluorenyl)dibenzothiophenyl]benzene; diphenyltriazinyl[(dimethylfluorenyl)benzselenophenyl]benzene; diphenyltriazinyl[(dimethylfluorenyl)dibenzofuranyl]benzene; diphenyltriazinyl[(dimethylfluorenyl)dibenzothiophenyl]benzene; diphenyltriazinyl[(dimethylfluorenyl)benzselenophenyl]benzene; diphenyltriazinyl[(dimethylfluorenyl)dibenzofuranyl]benzene; diphenyltriazinyl[(dimethylfluorenyl)dibenzothiophenyl]benzene; diphenyltriazinyl[(dimethylfluorenyl)benzselenophenyl]benzene; diphenyltriazinyl[(dimethylfluorenyl)dibenzofuranyl]benzene; diphenyltriazinyl[(dimethylfluorenyl)dibenzothiophenyl]benzene; diphenyltriazinyl[(dimethylfluorenyl)benzselenophenyl]benzene; diphenyltriazinyl[phenyl(dimethylfluorenyl)dibenzofuranyl]benzene; diphenyltriazinyl[phenyl(dimethylfluorenyl)dibenzothiophenyl]benzene; diphenyltriazinyl[phenyl(dimethylfluorenyl)benzselenophenyl]benzene; diphenyltriazinyl[phenyl(dimethylfluorenyl)dibenzofuranyl]benzene; diphenyltriazinyl[phenyl(dimethylfluorenyl)dibenzothiophenyl]benzene; diphenyltriazinyl[phenyl(dimethylfluorenyl)benzselenophenyl]benzene; diphenyltriazinyl[(dimethylfluorenyl)dibenzofuranyl]benzene; diphenyltriazinyl[(dimethylfluorenyl)dibenzothiophenyl]benzene; diphenyltriazinyl[(dimethylfluorenyl)benzselenophenyl]benzene; [(diphenyl-d₁₀)triazinyl][(dimethylfluorenyl)dibenzofuranyl]benzene; [(diphenyl-d₁₀)triazinyl][(dimethylfluorenyl)dibenzothiophenyl]benzene; [(diphenyl-d₁₀)triazinyl][(dimethylfluorenyl)benzselenophenyl]benzene; benzene-d₄[(diphenyl-d₁₀)triazinyl][(dimethylfluorenyl)dibenzofuranyl]; benzene-d₄[(diphenyl-d₁₀)triazinyl][(dimethylfluorenyl)dibenzothiophenyl]; benzene-d₄[(diphenyl-d₁₀)triazinyl][(dimethylfluorenyl)benzselenophenyl]; [(diphenyl-d₁₀)triazinyl]{[(dimethyl-d₆)fluorenyl]dibenzofuranyl}benzene; [(diphenyl-d₁₀)triazinyl]{[(dimethyl-d₆)fluorenyl]dibenzothiophenyl}benzene; [(diphenyl-d₁₀)triazinyl]{[(dimethyl-d₆)fluorenyl]benzselenophenyl}benzene; benzene-d₄[(diphenyl-d₁₀)triazinyl][(dimethylfluorenyl)dibenzofuranyl]; benzene-d₄[(diphenyl-d₁₀)triazinyl][(dimethylfluorenyl)dibenzothiophenyl]; benzene-d₄[(diphenyl-d₁₀)triazinyl][(dimethylfluorenyl)benzselenophenyl]

### (Group 19A)

phenyl[phenyl(biphenylyl)triazinyl]indenocarbazole; biphenylyl[phenyl(biphenylyl)triazinyl]indenocarbazole; biphenylyl[phenyl(biphenylyl)triazinyl]indenocarbazole; (dimethyl)(phenyl)[phenyl(biphenylyl)triazinyl]indenocarbazole; triphenyl[phenyl(biphenylyl)triazinyl]indenocarbazole; phenyl(dipyridinyl)[phenyl(biphenylyl)triazinyl]indenocarbazole; biphenylyl[phenyl(biphenylyl)triazinyl]indenocarbazole; phenyl[phenyl(biphenylyl)triazinyl]indenocarbazole; biphenylyl[phenyl(biphenylyl)triazinyl]indenocarbazole; terphenylyl[phenyl(biphenylyl)triazinyl]indenocarbazole; terphenylyl[phenyl(biphenylyl)triazinyl]indenocarbazole; [di(n-hexyl)](phenyl)[phenyl(biphenylyl)triazinyl]indenocarbazole; (dinaphthalenyl)(phenyl)[phenyl(biphenylyl)triazinyl]indenocarbazole; (diquinolinyl)(phenyl)[phenyl(biphenylyl)triazinyl]indenocarbazole; phenyl[phenyl(biphenylyl)triazinyl]indenocarbazole; biphenylyl[phenyl(biphenylyl)triazinyl]indenocarbazole; biphenylyl[phenyl(biphenylyl)triazinyl]indenocarbazole; phenyl[bis(dibenzofuranyl)][phenyl(biphenylyl)triazinyl]indenocarbazole; phenyl[bis(carbazolyl)][phenyl(biphenylyl)triazinyl]indenocarbazole; phenyl[bis(biphenylyl)triazinyl]indenocarbazole; phenyl[bis(biphenylyl)triazinyl]indenocarbazole; (biphenylyl)[bis(biphenylyl)triazinyl]indenocarbazole; (biphenylyl)[phenyl(biphenylyl)triazinyl]indenocarbazole; phenyl[bis(biphenylyl)triazinyl]indenocarbazole; phenyl[bis(biphenylyl)triazinyl]indenocarbazole; (biphenylyl)[bis(biphenylyl)triazinyl]indenocarbazole; (biphenylyl)[bis(biphenylyl)triazinyl]indenocarbazole; phenyl[bis(biphenylyl)triazinyl]indenocarbazole; (biphenylyl)[bis(biphenylyl)triazinyl]indenocarbazole; (biphenylyl)[bis(biphenylyl)triazinyl]indenocarbazole; phenyl[phenyl(terphenylyl)triazinyl]indenocarbazole; phenyl[bis(terphenylyl)triazinyl]indenocarbazole; phenyl[phenyl(biphenylyl)triazinyl]indenocarbazole; phenyl[bis(biphenylyl)triazinyl]indenocarbazole; phenyl[bis(biphenylyl)triazinyl]indenocarbazole; phenyl[bis(biphenylyl)triazinyl]indenocarbazole; (biphenylyl)[phenyl(biphenylyl)triazinyl]indenocarbazole; (dimethyl)(phenyl)[phenyl(biphenylyl)triazinyl]indenocarbazole; phenyl[phenyl(biphenylyl)triazinyl]indenocarbazole; phenyl[bis(biphenylyl)triazinyl]indenocarbazole; (biphenylyl)[phenyl(biphenylyl)triazinyl]indenocarbazole; (terphenylyl)[phenyl(biphenylyl)triazinyl]indenocarbazole; phenyl[phenyl(biphenylyl)triazinyl]indenocarbazole; phenyl[phenyl(biphenylyl)triazinyl]indenocarbazole; phenyl[phenyl(biphenylyl)triazinyl]indenocarbazole; phenyl[bis(biphenylyl)triazinyl]indenocarbazole; phenyl[bis(biphenylyl)triazinyl]indenocarbazole; phenyl[bis(biphenylyl)triazinyl]indenocarbazole; (biphenylyl)[phenyl(biphenylyl)triazinyl]indenocarbazole; (terphenylyl)[phenyl(biphenylyl)triazinyl]indenocarbazole; phenyl[phenyl(biphenylyl)triazinyl]indenocarbazole; phenyl[phenyl(biphenylyl)triazinyl]indenocarbazole; phenyl[phenyl(biphenylyl)triazinyl]indenocarbazole; phenyl[phenyl(terphenylyl)triazinyl]indenocarbazole; phenyl[bis(biphenylyl)triazinyl]indenocarbazole; phenyl[bis(biphenylyl)triazinyl]indenocarbazole; phenyl[bis(biphenylyl)triazinyl]indenocarbazole; (biphenylyl)[phenyl(biphenylyl)triazinyl]indenocarbazole; phenyl[phenyl(biphenylyl)triazinyl]indenocarbazole; phenyl[phenyl(biphenylyl)triazinyl]indenocarbazole; phenyl[phenyl(biphenylyl)triazinyl]indenocarbazole; phenyl[phenyl(terphenylyl)triazinyl]indenocarbazole; phenyl[phenyl(terphenylyl)triazinyl]indenocarbazole; (biphenylyl)[phenyl(biphenylyl)triazinyl]indenocarbazole; (biphenylyl)[phenyl(biphenylyl)triazinyl]indenocarbazole; phenyl[bis(biphenylyl)triazinyl]indenocarbazole; phenyl[bis(biphenylyl)triazinyl]indenocarbazole; phenyl[phenyl(biphenylyl)triazinyl]indenocarbazole; phenyl[phenyl(biphenylyl)triazinyl]indenocarbazole; phenyl[phenyl(terphenylyl)triazinyl]indenocarbazole; phenyl[(biphenylyl)(terphenylyl)triazinyl]indenocarbazole; (biphenylyl)[phenyl(biphenylyl)triazinyl]indenocarbazole; phenyl[bis(biphenylyl)triazinyl]indenocarbazole; phenyl[bis(biphenylyl)triazinyl]indenocarbazole; (biphenylyl)[phenyl(biphenylyl)triazinyl]indenocarbazole; phenyl(naphthalenyl)bi(carbazole); bi[(naphthalenyl)carbazole]; (biphenylyl)(naphthalenyl)bi(carbazole); (biphenylyl)(naphthalenyl)bi(carbazole); (terphenylyl)(naphthalenyl)bi(carbazole); [(naphthalenyl)phenyl](naphthalenyl)bi(carbazole); bi[(naphthalenyl)carbazole]; (phenanthrenyl)(naphthalenyl)bi(carbazole); (phenylanthracenyl)(naphthalenyl)bi(carbazole); phenyl(naphthalenyl)bi(carbazole); bi[(naphthalenyl)carbazole]; (biphenylyl)(naphthalenyl)bi(carbazole); (biphenylyl)(naphthalenyl)bi(carbazole); (terphenylyl)(naphthalenyl)bi(carbazole); [(naphthalenyl)phenyl](naphthalenyl)bi(carbazole); bi[(naphthalenyl)carbazole]; (phenanthrenyl)(naphthalenyl)bi(carbazole); (phenylanthracenyl)(naphthalenyl)bi(carbazole); phenyl(phenanthrenyl)bi(carbazole); (phenanthrenyl)(biphenylyl)bi(carbazole); (phenanthrenyl)(biphenylyl)bi(carbazole); (terphenylyl)(phenanthrenyl)bi(carbazole); (phenanthrenyl)[(naphthalenyl)phenyl]bi(carbazole); bi[(phenanthrenyl)carbazole]; (phenylanthracenyl)(phenanthrenyl)bi(carbazole); phenyl(naphthalenyl)bi(carbazole); bi[(naphthalenyl)carbazole]; phenyl(naphthalenyl)bi(carbazole); bi[(naphthalenyl)carbazole]; phenyl(phenanthrenyl)bi(carbazole); bi[(phenanthrenyl)carbazole]; phenyl(anthracenyl)bi(carbazole); bi[(anthracenyl)carbazole]; phenyl(naphthalenyl)bi(carbazole); bi[(naphthalenyl)carbazole]; phenyl(naphthalenyl)bi(carbazole); bi[(naphthalenyl)carbazole]; phenyl(phenanthrenyl)bi(carbazole); bi[(phenanthrenyl)carbazole]; phenyl(anthracenyl)bi(carbazole); bi[(anthracenyl)carbazole]; phenyl(naphthalenyl)bi(carbazole); bi[(naphthalenyl)carbazole]; phenyl(naphthalenyl)bi(carbazole); bi[(naphthalenyl)carbazole]; phenyl(phenanthrenyl)bi(carbazole); bi[(phenanthrenyl)carbazole]; phenyl(naphthalenyl)bi(carbazole); bi[(naphthalenyl)carbazole]; phenyl(naphthalenyl)bi(carbazole); phenyl(naphthalenyl)bi(carbazole); bi[(naphthalenyl)carbazole]; phenyl(naphthalenyl)bi(carbazole); phenyl(phenanthrenyl)bi(carbazole); bi[(phenanthrenyl)carbazole]; phenyl(phenanthrenyl)bi(carbazole); phenyl(anthracenyl)bi(carbazole); phenyl(naphthalenyl)bi(carbazole); phenyl(naphthalenyl)bi(carbazole); bi[(naphthalenyl)carbazole]; phenyl(naphthalenyl)bi(carbazole); phenyl(phenanthrenyl)bi(carbazole); phenyl(anthracenyl)bi(carbazole); phenyl(naphthalenyl)bi(carbazole); phenyl(naphthalenyl)bi(carbazole); bi[(naphthalenyl)carbazole]; phenyl(naphthalenyl)bi(carbazole); phenyl(naphthalenyl)bi(carbazole); bi[(naphthalenyl)carbazole]; phenyl(phenanthrenyl)bi(carbazole); phenyl(phenanthrenyl)bi(carbazole); bi[(phenanthrenyl)carbazole]; phenyl(anthracenyl)bi(carbazole); phenyl(anthracenyl)bi(carbazole); bi[(anthracenyl)carbazole]

### (Group 20A)

(dibenzofuranyl)(diphenyltriazinyl)biphenyl; (dibenzothiophenyl)(diphenyltriazinyl)biphenyl; (dibenzoselenophenyl)(diphenyltriazinyl)biphenyl; (dibenzofuranyl)(diphenyltriazinyl)terbenzene; (dibenzothiophenyl)(diphenyltriazinyl)terbenzene; (dibenzoselenophenyl)(diphenyltriazinyl)terbenzene; (dibenzofuranyl)(diphenyltriazinyl)terbenzene; (dibenzothiophenyl)(diphenyltriazinyl)terbenzene; (dibenzoselenophenyl)(diphenyltriazinyl)terbenzene; (phenyldibenzofuranyl)(diphenyltriazinyl)biphenyl; (phenyldibenzothiophenyl)(diphenyltriazinyl)biphenyl; (phenyldibenzoselenophenyl)(diphenyltriazinyl)biphenyl; [(biphenylyl)dibenzofuranyl](diphenyltriazinyl)biphenyl; [(biphenylyl)dibenzothiophenyl](diphenyltriazinyl)biphenyl; [(biphenylyl)dibenzoselenophenyl](diphenyltriazinyl)biphenyl; [(biphenylyl)dibenzofuranyl](diphenyltriazinyl)biphenyl; [(biphenylyl)dibenzothiophenyl](diphenyltriazinyl)biphenyl; [(biphenylyl)dibenzoselenophenyl](diphenyltriazinyl)biphenyl; (phenyldibenzofuranyl)(diphenyltriazinyl)terbenzene; (phenyldibenzothiophenyl)(diphenyltriazinyl)terbenzene; (phenyldibenzoselenophenyl)(diphenyltriazinyl)terbenzene; (phenyldibenzofuranyl)(diphenyltriazinyl)terbenzene; (phenyldibenzothiophenyl)(diphenyltriazinyl)terbenzene; (phenyldibenzoselenophenyl)(diphenyltriazinyl)terbenzene; [(biphenylyl)dibenzofuranyl](diphenyltriazinyl)terbenzene; [(biphenylyl)dibenzothiophenyl](diphenyltriazinyl)terbenzene; [(biphenylyl)dibenzoselenophenyl](diphenyltriazinyl)terbenzene; (dibenzofuranyl)[phenyl(biphenylyl)triazinyl]biphenyl; (dibenzothiophenyl)[phenyl(biphenylyl)triazinyl]biphenyl; (dibenzoselenophenyl)[phenyl(biphenylyl)triazinyl]biphenyl; (dibenzofuranyl)[phenyl(biphenylyl)triazinyl]biphenyl; (dibenzothiophenyl)[phenyl(biphenylyl)triazinyl]biphenyl; (dibenzoselenophenyl)[phenyl(biphenylyl)triazinyl]biphenyl; (dibenzofuranyl)[bis(biphenylyl)triazinyl]biphenyl; (dibenzothiophenyl)[bis(biphenylyl)triazinyl]biphenyl; (dibenzoselenophenyl)[bis(biphenylyl)triazinyl]biphenyl; (dibenzofuranyl)[phenyl(dimethylfluorenyl)triazinyl]biphenyl; (dibenzothiophenyl)[phenyl(dimethylfluorenyl)triazinyl]biphenyl; (dibenzoselenophenyl)[phenyl(dimethylfluorenyl)triazinyl]biphenyl; (phenyldibenzofuranyl)[phenyl(biphenylyl)triazinyl]biphenyl; (phenyldibenzothiophenyl)[phenyl(biphenylyl)triazinyl]biphenyl; (phenyldibenzoselenophenyl)[phenyl(biphenylyl)triazinyl]biphenyl; (phenyldibenzofuranyl)[phenyl(biphenylyl)triazinyl]biphenyl; (phenyldibenzothiophenyl)[phenyl(biphenylyl)triazinyl]biphenyl; (phenyldibenzoselenophenyl)[phenyl(biphenylyl)triazinyl]biphenyl; (phenyldibenzofuranyl)[phenyl(dimethylfluorenyl)triazinyl]biphenyl; (phenyldibenzothiophenyl)[phenyl(dimethylfluorenyl)triazinyl]biphenyl; (phenyldibenzoselenophenyl)[phenyl(dimethylfluorenyl)triazinyl]biphenyl; (dibenzofuranyl)[phenyl(biphenylyl)triazinyl]biphenyl; (dibenzothiophenyl)[phenyl(biphenylyl)triazinyl]biphenyl; (dibenzoselenophenyl)[phenyl(biphenylyl)triazinyl]biphenyl; (dibenzofuranyl)[phenyl(biphenylyl)triazinyl]terbenzene; (dibenzothiophenyl)[phenyl(biphenylyl)triazinyl]terbenzene; (dibenzoselenophenyl)[phenyl(biphenylyl)triazinyl]terbenzene; (dibenzofuranyl)[phenyl(dimethylfluorenyl)triazinyl]terbenzene; (dibenzothiophenyl)[phenyl(dimethylfluorenyl)triazinyl]terbenzene; (dibenzoselenophenyl)[phenyl(dimethylfluorenyl)triazinyl]terbenzene; [(biphenylyl)dibenzofuranyl][phenyl(biphenylyl)triazinyl]biphenyl; [(biphenylyl)dibenzothiophenyl][phenyl(biphenylyl)triazinyl]biphenyl; [(biphenylyl)dibenzoselenophenyl][phenyl(biphenylyl)triazinyl]biphenyl; [(biphenylyl)dibenzofuranyl][phenyl(biphenylyl)triazinyl]biphenyl; [(biphenylyl)dibenzothiophenyl][phenyl(biphenylyl)triazinyl]biphenyl; [(biphenylyl)dibenzoselenophenyl][phenyl(biphenylyl)triazinyl]biphenyl; [(biphenylyl)dibenzofuranyl][phenyl(dimethylfluorenyl)triazinyl]biphenyl; [(biphenylyl)dibenzothiophenyl][phenyl(dimethylfluorenyl)triazinyl]biphenyl; [(biphenylyl)dibenzoselenophenyl][phenyl(dimethylfluorenyl)triazinyl]biphenyl; (diphenyltriazinyl)[(biphenylyl)dibenzofuranyl]terbenzene; (diphenyltriazinyl)[(biphenylyl)dibenzothiophenyl]terbenzene; (diphenyltriazinyl)[(biphenylyl)dibenzoselenophenyl]terbenzene; (dibenzofuranyl)(diphenyltriazinyl)terbenzene; (dibenzothiophenyl)(diphenyltriazinyl)terbenzene; (dibenzoselenophenyl)(diphenyltriazinyl)terbenzene; (dibenzofuranyl)(diphenyltriazinyl)biphenyl; (dibenzothiophenyl)(diphenyltriazinyl)biphenyl; (dibenzoselenophenyl)(diphenyltriazinyl)biphenyl; (dibenzofuranyl)(diphenyltriazinyl)biphenyl; (dibenzothiophenyl)(diphenyltriazinyl)biphenyl; (dibenzoselenophenyl)(diphenyltriazinyl)biphenyl; (dibenzofuranyl)(diphenyltriazinyl)biphenyl; (dibenzothiophenyl)(diphenyltriazinyl)biphenyl; (dibenzoselenophenyl)(diphenyltriazinyl)biphenyl; (dibenzofuranyl)(diphenyltriazinyl)terbenzene; (dibenzothiophenyl)(diphenyltriazinyl)terbenzene; (dibenzoselenophenyl)(diphenyltriazinyl)terbenzene; (dibenzofuranyl)(diphenyltriazinyl)terbenzene; (dibenzothiophenyl)(diphenyltriazinyl)terbenzene; (dibenzoselenophenyl)(diphenyltriazinyl)terbenzene; (dibenzofuranyl)(diphenyltriazinyl)terbenzene; (dibenzothiophenyl)(diphenyltriazinyl)terbenzene; (dibenzoselenophenyl)(diphenyltriazinyl)terbenzene; (diphenyldibenzofuranyl)(diphenyltriazinyl)biphenyl; (diphenyldibenzothiophenyl)(diphenyltriazinyl)biphenyl; (diphenyldibenzoselenophenyl)(diphenyltriazinyl)biphenyl; (diphenyldibenzofuranyl)(diphenyltriazinyl)biphenyl; (diphenyldibenzothiophenyl)(diphenyltriazinyl)biphenyl; (diphenyldibenzoselenophenyl)(diphenyltriazinyl)biphenyl; (phenyldibenzofuranyl)(diphenyltriazinyl)biphenyl; (phenyldibenzothiophenyl)(diphenyltriazinyl)biphenyl; (phenyldibenzoselenophenyl)(diphenyltriazinyl)biphenyl; (phenyldibenzofuranyl)(diphenyltriazinyl)(pyridinyl)benzene; (phenyldibenzothiophenyl)(diphenyltriazinyl)(pyridinyl)benzene; (phenyldibenzoselenophenyl)(diphenyltriazinyl)(pyridinyl)benzene; (phenyldibenzofuranyl)(diphenyltriazinyl)(pyridinyl)benzene; (phenyldibenzothiophenyl)(diphenyltriazinyl)(pyridinyl)benzene; (phenyldibenzoselenophenyl)(diphenyltriazinyl)(pyridinyl)benzene; [(pyridinyl)dibenzofuranyl](diphenyltriazinyl)biphenyl; [(pyridinyl)dibenzothiophenyl](diphenyltriazinyl)biphenyl; [(pyridinyl)dibenzoselenophenyl](diphenyltriazinyl)biphenyl; (diphenyltriazinyl)(dibenzofuranyl)terbenzene; (diphenyltriazinyl)(dibenzothiophenyl)terbenzene; (diphenyltriazinyl)(dibenzoselenophenyl)terbenzene; (dibenzofuranyl)(diphenyltriazinyl)terbenzene; (dibenzothiophenyl)(diphenyltriazinyl)terbenzene; (dibenzoselenophenyl)(diphenyltriazinyl)terbenzene; (dibenzofuranyl)(diphenyltriazinyl)(dimethylfluorenyl)benzene; (dibenzothiophenyl)(diphenyltriazinyl)(dimethylfluorenyl)benzene; (dibenzoselenophenyl)(diphenyltriazinyl)(dimethylfluorenyl)benzene; (diphenyltriazinyl)(dimethylfluorenyl)biphenyl; (diphenyltriazinyl)(spiro[cyclopentanefluorene]yl)biphenyl; (diphenyltriazinyl)(dimethylfluorenyl)terbenzene; (diphenyltriazinyl)(diphenylfluorenyl)biphenyl; [phenyl(biphenylyl)triazinyl](dimethylfluorenyl)biphenyl; [phenyl(dimethylfluorenyl)triazinyl](dimethylfluorenyl)terbenzene; (diphenyltriazinyl)[bis(dimethylfluorenyl)]benzene; (diphenyltriazinyl)(dimethylfluorenyl)terbenzene; (diphenyltriazinyl)phenyldibenzofuran; (diphenyltriazinyl)phenyldibenzothiophene; (diphenyltriazinyl)phenyldibenzoselenophene; [phenyl(biphenylyl)triazinyl]phenyldibenzofuran; [phenyl(biphenylyl)triazinyl]phenyldibenzothiophene; [phenyl(biphenylyl)triazinyl]phenyldibenzoselenophene; [phenyl(biphenylyl)triazinyl]phenyldibenzofuran; [phenyl(biphenylyl)triazinyl]phenyldibenzothiophene; [phenyl(biphenylyl)triazinyl]phenyldibenzoselenophene; [bis(biphenylyl)triazinyl]phenyldibenzofuran; [bis(biphenylyl)triazinyl]phenyldibenzothiophene; [bis(biphenylyl)triazinyl]phenyldibenzoselenophene; [bis(biphenylyl)triazinyl]phenyldibenzofuran; [bis(biphenylyl)triazinyl]phenyldibenzothiophene; [bis(biphenylyl)triazinyl]phenyldibenzoselenophene; [bis(biphenylyl)triazinyl]phenyldibenzofuran; [bis(biphenylyl)triazinyl]phenyldibenzothiophene; [bis(biphenylyl)triazinyl]phenyldibenzoselenophene; (diphenyltriazinyl)(phenyldibenzofuranyl)benzene; (diphenyltriazinyl)(phenyldibenzothiophenyl)benzene; (diphenyltriazinyl)(phenyldibenzoselenophenyl)benzene; [phenyl(biphenylyl)triazinyl](phenyldibenzofuranyl)benzene; [phenyl(biphenylyl)triazinyl](phenyldibenzothiophenyl)benzene; [phenyl(biphenylyl)triazinyl](phenyldibenzoselenophenyl)benzene; [phenyl(biphenylyl)triazinyl](phenyldibenzofuranyl)benzene; [phenyl(biphenylyl)triazinyl](phenyldibenzothiophenyl)benzene; [phenyl(biphenylyl)triazinyl](phenyldibenzoselenophenyl)benzene; (diphenyltriazinyl)(phenyldibenzofuranyl)benzene; (diphenyltriazinyl)(phenyldibenzothiophenyl)benzene; (diphenyltriazinyl)(phenyldibenzoselenophenyl)benzene; [phenyl(biphenylyl)triazinyl](phenyldibenzofuranyl)benzene; [phenyl(biphenylyl)triazinyl](phenyldibenzothiophenyl)benzene; [phenyl(biphenylyl)triazinyl](phenyldibenzoselenophenyl)benzene; [phenyl(biphenylyl)triazinyl](phenyldibenzofuranyl)benzene; [phenyl(biphenylyl)triazinyl](phenyldibenzothiophenyl)benzene; [phenyl(biphenylyl)triazinyl](phenyldibenzoselenophenyl)benzene; (diphenyltriazinyl)(phenyldibenzofuranyl)biphenyl; (diphenyltriazinyl)(phenyldibenzothiophenyl)biphenyl; (diphenyltriazinyl)(phenyldibenzoselenophenyl)biphenyl; [phenyl(biphenylyl)triazinyl](phenyldibenzofuranyl)biphenyl; [phenyl(biphenylyl)triazinyl](phenyldibenzothiophenyl)biphenyl; [phenyl(biphenylyl)triazinyl](phenyldibenzoselenophenyl)biphenyl; [phenyl(biphenylyl)triazinyl](phenyldibenzofuranyl)biphenyl; [phenyl(biphenylyl)triazinyl](phenyldibenzothiophenyl)biphenyl; [phenyl(biphenylyl)triazinyl](phenyldibenzoselenophenyl)biphenyl; (diphenyltriazinyl)(phenyldibenzofuranyl)biphenyl; (diphenyltriazinyl)(phenyldibenzothiophenyl)biphenyl; (diphenyltriazinyl)(phenyldibenzoselenophenyl)biphenyl; [phenyl(biphenylyl)triazinyl](phenyldibenzofuranyl)biphenyl; [phenyl(biphenylyl)triazinyl](phenyldibenzothiophenyl)biphenyl; [phenyl(biphenylyl)triazinyl](phenyldibenzoselenophenyl)biphenyl; [phenyl(biphenylyl)triazinyl](phenyldibenzofuranyl)biphenyl; [phenyl(biphenylyl)triazinyl](phenyldibenzothiophenyl)biphenyl; [phenyl(biphenylyl)triazinyl](phenyldibenzoselenophenyl)biphenyl; (diphenyltriazinyl)(phenyldibenzofuranyl)biphenyl; (diphenyltriazinyl)(phenyldibenzothiophenyl)biphenyl; (diphenyltriazinyl)(phenyldibenzoselenophenyl)biphenyl; [phenyl(biphenylyl)triazinyl](phenyldibenzofuranyl)biphenyl; [phenyl(biphenylyl)triazinyl](phenyldibenzothiophenyl)biphenyl; [phenyl(biphenylyl)triazinyl](phenyldibenzoselenophenyl)biphenyl; [phenyl(biphenylyl)triazinyl](phenyldibenzofuranyl)biphenyl; [phenyl(biphenylyl)triazinyl](phenyldibenzothiophenyl)biphenyl; [phenyl(biphenylyl)triazinyl](phenyldibenzoselenophenyl)biphenyl; (diphenyltriazinyl)(biphenylyl)dibenzofuran; (diphenyltriazinyl)(biphenylyl)dibenzothiophene; (diphenyltriazinyl)(biphenylyl)dibenzoselenophene; [phenyl(biphenylyl)triazinyl](biphenylyl)dibenzofuran; [phenyl(biphenylyl)triazinyl](biphenylyl)dibenzothiophene; [phenyl(biphenylyl)triazinyl](biphenylyl)dibenzoselenophene; [phenyl(biphenylyl)triazinyl](biphenylyl)dibenzofuran; [phenyl(biphenylyl)triazinyl](biphenylyl)dibenzothiophene; [phenyl(biphenylyl)triazinyl](biphenylyl)dibenzoselenophene; (diphenyltriazinyl)[(biphenylyl)dibenzofuranyl]benzene; (diphenyltriazinyl)[(biphenylyl)dibenzothiophenyl]benzene; (diphenyltriazinyl)[(biphenylyl)dibenzoselenophenyl]benzene; [phenyl(biphenylyl)triazinyl][(biphenylyl)dibenzofuranyl]benzene; [phenyl(biphenylyl)triazinyl][(biphenylyl)dibenzothiophene]benzene; [phenyl(biphenylyl)triazinyl][(biphenylyl)dibenzoselenophene]benzene; [phenyl(biphenylyl)triazinyl][(biphenylyl)dibenzofuranyl]benzene; [phenyl(biphenylyl)triazinyl][(biphenylyl)dibenzothiophene]benzene; [phenyl(biphenylyl)triazinyl][(biphenylyl)dibenzoselenophene]benzene; (diphenyltriazinyl)[(biphenylyl)dibenzofuranyl]benzene; (diphenyltriazinyl)[(biphenylyl)dibenzothiophenyl]benzene; (diphenyltriazinyl)[(biphenylyl)dibenzoselenophenyl]benzene; [phenyl(biphenylyl)triazinyl][(biphenylyl)dibenzofuranyl]benzene; [phenyl(biphenylyl)triazinyl][(biphenylyl)dibenzothiophenyl]benzene; [phenyl(biphenylyl)triazinyl][(biphenylyl)dibenzoselenophenyl]benzene; [phenyl(biphenylyl)triazinyl][(biphenylyl)dibenzofuranyl]benzene; [phenyl(biphenylyl)triazinyl][(biphenylyl)dibenzothiophenyl]benzene; [phenyl(biphenylyl)triazinyl][(biphenylyl)dibenzoselenophenyl]benzene; (diphenyltriazinyl)(biphenylyl)dibenzofuran; (diphenyltriazinyl)(biphenylyl)dibenzothiophene; (diphenyltriazinyl)(biphenylyl)dibenzoselenophene; [phenyl(biphenylyl)triazinyl](biphenylyl)dibenzofuran; [phenyl(biphenylyl)triazinyl](biphenylyl)dibenzothiophene; [phenyl(biphenylyl)triazinyl](biphenylyl)dibenzoselenophene; [phenyl(biphenylyl)triazinyl](biphenylyl)dibenzofuran; [phenyl(biphenylyl)triazinyl](biphenylyl)dibenzothiophene; [phenyl(biphenylyl)triazinyl](biphenylyl)dibenzoselenophene; (diphenyltriazinyl)[(biphenylyl)dibenzofuranyl]benzene; (diphenyltriazinyl)[(biphenylyl)dibenzothiophenyl]benzene; (diphenyltriazinyl)[(biphenylyl)dibenzoselenophenyl]benzene; [phenyl(biphenylyl)triazinyl][(biphenylyl)dibenzofuranyl]benzene; [phenyl(biphenylyl)triazinyl][(biphenylyl)dibenzothiophenyl]benzene; [phenyl(biphenylyl)triazinyl][(biphenylyl)dibenzoselenophenyl]benzene; (diphenyltriazinyl)[(biphenylyl)dibenzofuranyl]benzene; (diphenyltriazinyl)[(biphenylyl)dibenzothiophenyl]benzene; (diphenyltriazinyl)[(biphenylyl)dibenzoselenophenyl]benzene; (diphenyltriazinyl)[(biphenylyl)dibenzofuranyl]benzene; (diphenyltriazinyl)[(biphenylyl)dibenzothiophenyl]benzene; (diphenyltriazinyl)[(biphenylyl)dibenzoselenophenyl]benzene; [phenyl(biphenylyl)triazinyl][(biphenylyl)dibenzofuranyl]benzene; [phenyl(biphenylyl)triazinyl][(biphenylyl)dibenzothiophenyl]benzene; [phenyl(biphenylyl)triazinyl][(biphenylyl)dibenzoselenophenyl]benzene; [phenyl(biphenylyl)triazinyl][(biphenylyl)dibenzofuranyl]benzene; [phenyl(biphenylyl)triazinyl][(biphenylyl)dibenzothiophenyl]benzene; [phenyl(biphenylyl)triazinyl][(biphenylyl)dibenzoselenophenyl]benzene; [phenyl(dimethylfluorenyl)triazinyl](phenyldibenzofuran); [phenyl(dimethylfluorenyl)triazinyl](phenyldibenzothiophene); [phenyl(dimethylfluorenyl)triazinyl](phenyldibenzoselenophene); [(biphenylyl)(dimethylfluorenyl)triazinyl](phenyldibenzofuran); [(biphenylyl)(dimethylfluorenyl)triazinyl](phenyldibenzothiophene); [(biphenylyl)(dimethylfluorenyl)triazinyl](phenyldibenzoselenophene); [(biphenylyl)(dimethylfluorenyl)triazinyl](phenyldibenzofuran); [(biphenylyl)(dimethylfluorenyl)triazinyl](phenyldibenzothiophene); [(biphenylyl)(dimethylfluorenyl)triazinyl](phenyldibenzoselenophene); [phenyl(dimethylfluorenyl)triazinyl](phenyldibenzofuranyl)benzene; [phenyl(dimethylfluorenyl)triazinyl](phenyldibenzothiophenyl)benzene; [phenyl(dimethylfluorenyl)triazinyl](phenyldibenzoselenophenyl)benzene; [bis(dimethylfluorenyl)triazinyl](phenyldibenzofuranyl)benzene; [bis(dimethylfluorenyl)triazinyl](phenyldibenzothiophenyl)benzene; [bis(dimethylfluorenyl)triazinyl](phenyldibenzoselenophenyl)benzene; [phenyl(dimethylfluorenyl)triazinyl](biphenylyl)dibenzofuran; [phenyl(dimethylfluorenyl)triazinyl](biphenylyl)dibenzothiophene; [phenyl(dimethylfluorenyl)triazinyl](biphenylyl)dibenzoselenophene; [phenyl(dimethylfluorenyl)triazinyl](biphenylyl)dibenzofuran; [phenyl(dimethylfluorenyl)triazinyl](biphenylyl)dibenzothiophene; [phenyl(dimethylfluorenyl)triazinyl](biphenylyl)dibenzoselenophene; [phenyl(dimethylfluorenyl)triazinyl][(biphenylyl)dibenzofuranyl]benzene; [phenyl(dimethylfluorenyl)triazinyl][(biphenylyl)dibenzothiophenyl]benzene; [phenyl(dimethylfluorenyl)triazinyl][(biphenylyl)dibenzoselenophenyl]benzene; [phenyl(dimethylfluorenyl)triazinyl][(biphenylyl)dibenzofuranyl]benzene; [phenyl(dimethylfluorenyl)triazinyl][(biphenylyl)dibenzothiophenyl]benzene; [phenyl(dimethylfluorenyl)triazinyl][(biphenylyl)dibenzoselenophenyl]benzene; [phenyl(dimethylfluorenylphenyl)triazinyl](phenyldibenzofuranyl)benzene; [phenyl(dimethylfluorenylphenyl)triazinyl](phenyldibenzothiophenyl)benzene; [phenyl(dimethylfluorenylphenyl)triazinyl](phenyldibenzoselenophenyl)benzene; [phenyl(dimethylfluorenyl)triazinyl]phenyldibenzofuran; [phenyl(dimethylfluorenyl)triazinyl]phenyldibenzothiophene; [phenyl(dimethylfluorenyl)triazinyl]phenyldibenzoselenophene; [phenyl(dimethylfluorenyl)triazinyl](biphenylyl)dibenzofuran; [phenyl(dimethylfluorenyl)triazinyl](biphenylyl)dibenzothiophene; [phenyl(dimethylfluorenyl)triazinyl](biphenylyl)dibenzoselenophene; [phenyl(dimethylfluorenyl)triazinyl][(biphenylyl)dibenzofuranyl]benzene; [phenyl(dimethylfluorenyl)triazinyl][(biphenylyl)dibenzothiophenyl]benzene; [phenyl(dimethylfluorenyl)triazinyl][(biphenylyl)dibenzoselenophenyl]benzene; [phenyl(dimethylfluorenylphenyl)triazinyl](phenyldibenzofuranyl)benzene; [phenyl(dimethylfluorenylphenyl)triazinyl](phenyldibenzothiophenyl)benzene; [phenyl(dimethylfluorenylphenyl)triazinyl](phenyldibenzoselenophenyl)benzene; [phenyl(dimethylfluorenyl)triazinyl](phenyldibenzofuran); [phenyl(dimethylfluorenyl)triazinyl](phenyldibenzothiophene); [phenyl(dimethylfluorenyl)triazinyl](phenyldibenzoselenophene); phenyl(diphenyltriazinyl)dibenzofuran; phenyl(diphenyltriazinyl)dibenzothiophene; phenyl(diphenyltriazinyl)dibenzoselenophene; phenyl[(diphenyltriazinyl)phenyl]dibenzofuran; phenyl[(diphenyltriazinyl)phenyl]dibenzothiophene; phenyl[(diphenyltriazinyl)phenyl]dibenzoselenophene; (biphenylyl)[(diphenyltriazinyl)phenyl]dibenzofuran; (biphenylyl)[(diphenyltriazinyl)phenyl]dibenzothiophene; (biphenylyl)[(diphenyltriazinyl)phenyl]dibenzoselenophene; diphenyl[(diphenyltriazinyl)phenyl]dibenzofuran; diphenyl[(diphenyltriazinyl)phenyl]dibenzothiophene; diphenyl[(diphenyltriazinyl)phenyl]dibenzoselenophene; phenyl(diphenyltriazinyl)dibenzofuran; phenyl(diphenyltriazinyl)dibenzothiophene; phenyl(diphenyltriazinyl)dibenzoselenophene; (biphenylyl)(diphenyltriazinyl)dibenzofuran; (biphenylyl)(diphenyltriazinyl)dibenzothiophene; (biphenylyl)(diphenyltriazinyl)dibenzoselenophene; (biphenylyl)(diphenyltriazinyl)dibenzofuran; (biphenylyl)(diphenyltriazinyl)dibenzothiophene; (biphenylyl)(diphenyltriazinyl)dibenzoselenophene; diphenyl[(diphenyltriazinyl)phenyl]dibenzofuran; diphenyl[(diphenyltriazinyl)phenyl]dibenzothiophene; diphenyl[(diphenyltriazinyl)phenyl]dibenzoselenophene; (biphenylyl)[(diphenyltriazinyl)phenyl]dibenzofuran; (biphenylyl)[(diphenyltriazinyl)phenyl]dibenzothiophene; (biphenylyl)[(diphenyltriazinyl)phenyl]dibenzoselenophene; phenyl(diphenyltriazinyl)dibenzofuran; phenyl(diphenyltriazinyl)dibenzothiophene; phenyl(diphenyltriazinyl)dibenzoselenophene; phenyl[(diphenyltriazinyl)phenyl]dibenzofuran; phenyl[(diphenyltriazinyl)phenyl]dibenzothiophene; phenyl[(diphenyltriazinyl)phenyl]dibenzoselenophene; phenyl[(diphenyltriazinyl)phenyl]dibenzofuran; phenyl[(diphenyltriazinyl)phenyl]dibenzothiophene; phenyl[(diphenyltriazinyl)phenyl]dibenzoselenophene; (biphenylyl)[(diphenyltriazinyl)phenyl]dibenzofuran; (biphenylyl)[(diphenyltriazinyl)phenyl]dibenzothiophene; (biphenylyl)[(diphenyltriazinyl)phenyl]dibenzoselenophene; (biphenylyl)[(diphenyltriazinyl)phenyl]dibenzofuran; (biphenylyl)[(diphenyltriazinyl)phenyl]dibenzothiophene; (biphenylyl)[(diphenyltriazinyl)phenyl]dibenzoselenophene; phenyl[(diphenyltriazinyl)phenyl]dibenzofuran; phenyl[(diphenyltriazinyl)phenyl]dibenzothiophene; phenyl[(diphenyltriazinyl)phenyl]dibenzoselenophene; (diphenyltriazinyl)(phenyldibenzofuranyl)pyridine; (diphenyltriazinyl)(phenyldibenzothiophenyl)pyridine; (diphenyltriazinyl)(phenyldibenzoselenophenyl)pyridine; [phenyl(phenylpyridinyl)triazinyl](phenyldibenzofuranyl)pyridine; [phenyl(phenylpyridinyl)triazinyl](phenyldibenzothiophenyl)pyridine; [phenyl(phenylpyridinyl)triazinyl](phenyldibenzoselenophenyl)pyridine; [phenyl(phenylpyridinyl)triazinyl](phenyldibenzofuranyl)benzene; [phenyl(phenylpyridinyl)triazinyl](phenyldibenzothiophenyl)benzene; [phenyl(phenylpyridinyl)triazinyl](phenyldibenzoselenophenyl)benzene; (diphenyltriazinyl)[(biphenylyl)dibenzofuranyl)]pyridine; (diphenyltriazinyl)[(biphenylyl)dibenzothiophenyl)]pyridine; (diphenyltriazinyl)[(biphenylyl)dibenzoselenophenyl)]pyridine; phenyl[phenyl(dimethylfluorenyl)triazinyl]dibenzofuran; phenyl[phenyl(dimethylfluorenyl)triazinyl]dibenzothiophene; phenyl[phenyl(dimethylfluorenyl)triazinyl]dibenzoselenophene; phenyl[(biphenylyl)(dimethylfluorenyl)triazinyl]dibenzofuran; phenyl[(biphenylyl)(dimethylfluorenyl)triazinyl]dibenzothiophene; phenyl[(biphenylyl)(dimethylfluorenyl)triazinyl]dibenzoselenophene; phenyl[phenyl(dimethylfluorenyl)triazinyl]dibenzofuran; phenyl[phenyl(dimethylfluorenyl)triazinyl]dibenzothiophene; phenyl[phenyl(dimethylfluorenyl)triazinyl]dibenzoselenophene; phenyl[phenyl(spiro[cyclohexanefluorene]yl)triazinyl]dibenzofuran; phenyl[phenyl(spiro[cyclohexanefluorene]yl)triazinyl]dibenzothiophene; phenyl[phenyl(spiro[cyclohexanefluorene]yl)triazinyl]dibenzoselenophene; phenyl[phenyl(spirobi[9H-fluorene]yl)triazinyl]dibenzofuran; phenyl[phenyl(spirobi[9H-fluorene]yl)triazinyl]dibenzothiophene; phenyl[phenyl(spirobi[9H-fluorene]yl)triazinyl]dibenzoselenophene; benzene-d₃[(diphenyl-d₁₀)triazinyl][(phenyl-d₅)dibenzofuranyl-d₆]; benzene-d₃[(diphenyl-d₁₀)triazinyl][(phenyl-d₅)dibenzothiophenyl-d₆]; benzene-d₃[(diphenyl-d₁₀)triazinyl][(phenyl-d₅)dibenzoselenophenyl-d₆]; benzene-d₃[(diphenyl-d₁₀)triazinyl][(biphenylyl-d₉)dibenzofuranyl-d₆]; benzene-d₃[(diphenyl-d₁₀)triazinyl][(biphenylyl-de)dibenzothiophenyl-d₆]; benzene-d₃[(diphenyl-d₁₀)triazinyl][(biphenylyl-d₉)dibenzoselenophenyl-d₆]; phenyl{phenyl[(dimethyl-d₆)fluorenyl]triazinyl}dibenzofuran; phenyl{phenyl[(dimethyl-d₆)fluorenyl]triazinyl}dibenzothiophene; phenyl{phenyl[(dimethyl-d₆)fluorenyl]triazinyl}dibenzoselenophene; benzene-d₃[(diphenyl-d₁₀)triazinyl][(biphenylyl-d₉)dibenzofuranyl-d₆]; benzene-d₃[(diphenyl-d₁₀)triazinyl][(biphenylyl-d₉)dibenzothiophenyl-d₆]; benzene-d₃[(diphenyl-d₁₀)triazinyl][(biphenylyl-d₉)dibenzoselenophenyl-d₆]; [(diphenyl-d₁₀)triazinyl][(biphenylyl)dibenzofuranyl]benzene; [(diphenyl-d₁₀)triazinyl][(biphenylyl)dibenzothiophenyl]benzene; [(diphenyl-d₁₀)triazinyl][(biphenylyl)dibenzoselenophenyl]benzene; (diphenyltriazinyl)[(biphenylyl)dibenzofuranyl]benzene; (diphenyltriazinyl)[(biphenylyl)dibenzothiophenyl]benzene; (diphenyltriazinyl)[(biphenylyl)dibenzoselenophenyl]benzene; (difluorophenyltriazinyl)[(biphenylyl)dibenzofuranyl]benzene; (difluorophenyltriazinyl)[(biphenylyl)dibenzothiophenyl]benzene; (difluorophenyltriazinyl)[(biphenylyl)dibenzoselenophenyl]benzene; (diphenyltriazinyl)[(dimethylfluorenyl)dibenzofuranyl]benzene; (diphenyltriazinyl)[(dimethylfluorenyl)dibenzothiophenyl]benzene; (diphenyltriazinyl)[(dimethylfluorenyl)dibenzoselenophenyl]benzene; (diphenyltriazinyl)[(dimethylfluorenyl)dibenzofuranyl]biphenyl; (diphenyltriazinyl)[(dimethylfluorenyl)dibenzothiophenyl]biphenyl; (diphenyltriazinyl)[(dimethylfluorenyl)dibenzoselenophenyl]biphenyl; (diphenyltriazinyl)[(dimethylfluorenyl)dibenzofuranyl]biphenyl; (diphenyltriazinyl)[(dimethylfluorenyl)dibenzothiophenyl]biphenyl; (diphenyltriazinyl)[(dimethylfluorenyl)dibenzoselenophenyl]biphenyl; (diphenyltriazinyl)[(dimethylfluorenyl)dibenzofuranyl]biphenyl; (diphenyltriazinyl)[(dimethylfluorenyl)dibenzothiophenyl]biphenyl; (diphenyltriazinyl)[(dimethylfluorenyl)dibenzoselenophenyl]biphenyl; (diphenyltriazinyl)[(dimethylfluorenyl)dibenzofuranyl]pyridine; (diphenyltriazinyl)[(dimethylfluorenyl)dibenzothiophenyl]pyridine; (diphenyltriazinyl)[(dimethylfluorenyl)dibenzoselenophenyl]pyridine; (diphenyltriazinyl)[(dimethylfluorenyl)dibenzofuranyl]benzene; (diphenyltriazinyl)[(dimethylfluorenyl)dibenzothiophenyl]benzene; (diphenyltriazinyl)[(dimethylfluorenyl)dibenzoselenophenyl]benzene; (diphenyltriazinyl)[(dimethylfluorenyl)dibenzofuranyl]benzene; (diphenyltriazinyl)[(dimethylfluorenyl)dibenzothiophenyl]benzene; (diphenyltriazinyl)[(dimethylfluorenyl)dibenzoselenophenyl]benzene; (diphenyltriazinyl)[(dimethylfluorenyl)dibenzofuranyl]benzene; (diphenyltriazinyl)[(dimethylfluorenyl)dibenzothiophenyl]benzene; (diphenyltriazinyl)[(dimethylfluorenyl)dibenzoselenophenyl]benzene; (diphenyltriazinyl)[(dimethylfluorenyl)dibenzofuranyl]biphenyl; (diphenyltriazinyl)[(dimethylfluorenyl)dibenzothiophenyl]biphenyl; (diphenyltriazinyl)[(dimethylfluorenyl)dibenzoselenophenyl]biphenyl; [phenyl(biphenylyl)triazinyl][(dimethylfluorenyl)dibenzofuranyl]benzene; [phenyl(biphenylyl)triazinyl][(dimethylfluorenyl)dibenzothiophenyl]benzene; [phenyl(biphenylyl)triazinyl][(dimethylfluorenyl)dibenzoselenophenyl]benzene; [phenyl(biphenylyl)triazinyl][(dimethylfluorenyl)dibenzofuranyl]benzene; [phenyl(biphenylyl)triazinyl][(dimethylfluorenyl)dibenzothiophenyl]benzene; [phenyl(biphenylyl)triazinyl][(dimethylfluorenyl)dibenzoselenophenyl]benzene; [phenyl(dimethylfluorenyl)triazinyl][(dimethylfluorenyl)dibenzofuranyl]benzene; [phenyl(dimethylfluorenyl)triazinyl][(dimethylfluorenyl)dibenzothiophenyl]benzene; [phenyl(dimethylfluorenyl)triazinyl][(dimethylfluorenyl)dibenzoselenophenyl]benzene; (diphenyltriazinyl)[(dimethylfluorenyl)dibenzofuranyl]benzene; (diphenyltriazinyl)[(dimethylfluorenyl)dibenzothiophenyl]benzene; (diphenyltriazinyl)[(dimethylfluorenyl)dibenzoselenophenyl]benzene; (diphenyltriazinyl)[bis(dimethylfluorenyl)dibenzofuranyl]benzene; (diphenyltriazinyl)[bis(dimethylfluorenyl)dibenzothiophenyl]benzene; (diphenyltriazinyl)[bis(dimethylfluorenyl)dibenzoselenophenyl]benzene; [phenyl(terphenylyl)triazinyl][(dimethylfluorenyl)dibenzofuranyl]benzene; [phenyl(terphenylyl)triazinyl][(dimethylfluorenyl)dibenzothiophenyl]benzene; [phenyl(terphenylyl)triazinyl][(dimethylfluorenyl)dibenzoselenophenyl]benzene; (diphenyltriazinyl)[(spiro[cyclopentanefluorene]yl)dibenzofuranyl]benzene; (diphenyltriazinyl)[(spiro[cyclopentanefluorene]yl)dibenzothiophenyl]benzene; (diphenyltriazinyl)[(spiro[cyclopentanefluorene]yl)dibenzoselenophenyl]benzene; (diphenyltriazinyl)[(diphenylfluorenyl)dibenzofuranyl]benzene; (diphenyltriazinyl)[(diphenylfluorenyl)dibenzothiophenyl]benzene; (diphenyltriazinyl)[(diphenylfluorenyl)dibenzoselenophenyl]benzene; (diphenyltriazinyl)[(diphenylfluorenyl)dibenzofuranyl]benzene; (diphenyltriazinyl)[(diphenylfluorenyl)dibenzothiophenyl]benzene; (diphenyltriazinyl)[(diphenylfluorenyl)dibenzoselenophenyl]benzene; (diphenyltriazinyl)[phenyl(dimethylfluorenyl)dibenzofuranyl]benzene; (diphenyltriazinyl)[phenyl(dimethylfluorenyl)dibenzothiophenyl]benzene; (diphenyltriazinyl)[phenyl(dimethylfluorenyl)dibenzoselenophenyl]benzene; (diphenyltriazinyl)[phenyl(dimethylfluorenyl)dibenzofuranyl]benzene; (diphenyltriazinyl)[phenyl(dimethylfluorenyl)dibenzothiophenyl]benzene; (diphenyltriazinyl)[phenyl(dimethylfluorenyl)dibenzoselenophenyl]benzene; (diphenyltriazinyl)[(phenyldimethylfluorenyl)dibenzofuranyl]benzene; (diphenyltriazinyl)[(phenyldimethylfluorenyl)dibenzothiophenyl]benzene; (diphenyltriazinyl)[(phenyldimethylfluorenyl)dibenzoselenophenyl]benzene; (diphenyltriazinyl){[(dimethyl-d₆)fluorenyl]dibenzofuranyl}benzene; (diphenyltriazinyl){[(dimethyl-d₆)fluorenyl]dibenzothiophenyl}benzene; (diphenyltriazinyl){[(dimethyl-d₆)fluorenyl]benzselenophenyl}benzene; (diphenyltriazinyl)[(dimethylfluorenyl)dibenzofuranyl]benzene; (diphenyltriazinyl)[(dimethylfluorenyl)dibenzothiophenyl]benzene; (diphenyltriazinyl)[(dimethylfluorenyl)dibenzoselenophenyl]benzene; (diphenyltriazinyl)[(dimethylfluorenyl)dibenzofuranyl]benzene; (diphenyltriazinyl)[(dimethylfluorenyl)dibenzothiophenyl]benzene; (diphenyltriazinyl)[(dimethylfluorenyl)dibenzoselenophenyl]benzene; (diphenyltriazinyl)[(dimethylfluorenyl)dibenzofuranyl]benzene; (diphenyltriazinyl)[(dimethylfluorenyl)dibenzothiophenyl]benzene; (diphenyltriazinyl)[(dimethylfluorenyl)dibenzoselenophenyl]benzene; (diphenyltriazinyl)[(dimethylfluorenyl)dibenzofuranyl]benzene; (diphenyltriazinyl)[(dimethylfluorenyl)dibenzothiophenyl]benzene; (diphenyltriazinyl)[(dimethylfluorenyl)dibenzoselenophenyl]benzene; (diphenyltriazinyl)[phenyl(dimethylfluorenyl)dibenzofuranyl]benzene; (diphenyltriazinyl)[phenyl(dimethylfluorenyl)dibenzothiophenyl]benzene; (diphenyltriazinyl)[phenyl(dimethylfluorenyl)dibenzoselenophenyl]benzene; (diphenyltriazinyl)[phenyl(dimethylfluorenyl)dibenzofuranyl]benzene; (diphenyltriazinyl)[phenyl(dimethylfluorenyl)dibenzothiophenyl]benzene; (diphenyltriazinyl)[phenyl(dimethylfluorenyl)dibenzoselenophenyl]benzene; (diphenyltriazinyl)[(dimethylfluorenyl)dibenzofuranyl]benzene; (diphenyltriazinyl)[(dimethylfluorenyl)dibenzothiophenyl]benzene; (diphenyltriazinyl)[(dimethylfluorenyl)dibenzoselenophenyl]benzene; [(diphenyl-d₁₀)triazinyl][(dimethylfluorenyl)dibenzofuranyl]benzene; [(diphenyl-d₁₀)triazinyl][(dimethylfluorenyl)dibenzothiophenyl]benzene; [(diphenyl-d₁₀)triazinyl][(dimethylfluorenyl)dibenzoselenophenyl]benzene; benzene-d₃[(diphenyl-d₁₀)triazinyl][(dimethylfluorenyl)dibenzofuranyl]; benzene-d₃[(diphenyl-d₁₀)triazinyl][(dimethylfluorenyl)dibenzothiophenyl]; benzene-d₃[(diphenyl-d₁₀)triazinyl][(dimethylfluorenyl)dibenzoselenophenyl]; [(diphenyl-d₁₀)triazinyl]{[(dimethyl-d₆)fluorenyl]dibenzofuranyl}benzene; [(diphenyl-d₁₀)triazinyl]{[(dimethyl-d₆)fluorenyl]dibenzothiophenyl}benzene; [(diphenyl-d₁₀)triazinyl]{[(dimethyl-d₆)fluorenyl]dibenzoselenophenyl}benzene; benzene-d₄[(diphenyl-d₁₀)triazinyl][(dimethylfluorenyl)dibenzofuranyl]; benzene-d₄(diphenyl-d₁₀)triazinyl][(dimethylfluorenyl)dibenzothiophenyl]; benzene-d₄[(diphenyl-d₁₀)triazinyl][(dimethylfluorenyl)dibenzoselenophenyl]; phenyl(triphenylenyl)bi[carbazole]; phenyl(triphenylenyl)bi[carbazole]; phenyl(triphenylenyl)bi[carbazole]; phenyl(dimethylfluorenyl)bi[carbazole]; phenyl(dimethylfluorenyl)bi[carbazole]; phenyl(dimethylfluorenyl)bi[carbazole]; phenyl(dimethylfluorenyl)bi[carbazole]; phenyl(dimethylfluorenyl)bi[carbazole]; phenyl(dimethylfluorenyl)bi[carbazole]; (biphenylyl)(dimethylfluorenyl)bi[carbazole]; (biphenylyl)(dimethylfluorenyl)bi[carbazole]; (terphenylyl)(dimethylfluorenyl)bi[carbazole]; diphenylter[carbazole]; diphenylter[carbazole]diphenylter[carbazole]phenyl(dibenzofuranyl)bi[carbazole]; phenyl(dibenzothiophenyl)bi[carbazole]; phenyl(dibenzoselenophenyl)bi[carbazole]; phenyl(dibenzofuranyl)bi[carbazole]; phenyl(dibenzothiophenyl)bi[carbazole]; phenyl(dibenzoselenophenyl)bi[carbazole]; phenyl(dibenzofuranyl)bi[carbazole]; phenyl(dibenzothiophenyl)bi[carbazole]; phenyl(dibenzoselenophenyl)bi[carbazole]; phenyl(dibenzofuranyl)bi[carbazole]; phenyl(dibenzothiophenyl)bi[carbazole]; phenyl(dibenzoselenophenyl)bi[carbazole]; phenyl(dibenzofuranyl)bi[carbazole]; phenyl(dibenzothiophenyl)bi[carbazole]; phenyl(dibenzoselenophenyl)bi[carbazole]; phenyl(dibenzofuranyl)bi[carbazole]; phenyl(dibenzothiophenyl)bi[carbazole]; phenyl(dibenzoselenophenyl)bi[carbazole]; phenyl(dibenzofuranyl)bi[carbazole]; phenyl(dibenzothiophenyl)bi[carbazole]; phenyl(dibenzoselenophenyl)bi[carbazole]; phenyl(dibenzofuranyl)bi[carbazole]; phenyl(dibenzothiophenyl)bi[carbazole]; phenyl(dibenzoselenophenyl)bi[carbazole]; (biphenylyl)(dibenzofuranyl)bi[carbazole]; (biphenylyl)(dibenzothiophenyl)bi[carbazole]; (biphenylyl)(dibenzoselenophenyl)bi[carbazole]; (biphenylyl)(dibenzofuranyl)bi[carbazole]; (biphenylyl)(dibenzothiophenyl)bi[carbazole]; (biphenylyl)(dibenzoselenophenyl)bi[carbazole]; (biphenylyl)(dibenzofuranyl)bi[carbazole]; (biphenylyl)(dibenzothiophenyl)bi[carbazole]; (biphenylyl)(dibenzoselenophenyl)bi[carbazole]; phenyl(phenyldibenzofuranyl)bi[carbazole]; phenyl(phenyldibenzothiophenyl)bi[carbazole]; phenyl(phenyldibenzoselenophenyl)bi[carbazole]; phenyl(dibenzofuranyl)bi[carbazole]; phenyl(dibenzothiophenyl)bi[carbazole]; phenyl(dibenzoselenophenyl)bi[carbazole]; phenyl(dibenzofuranyl)bi[carbazole]; phenyl(dibenzothiophenyl)bi[carbazole]; phenyl(dibenzoselenophenyl)bi[carbazole]; phenyl(dibenzofuranyl)bi[carbazole]; phenyl(dibenzothiophenyl)bi[carbazole]; phenyl(dibenzoselenophenyl)bi[carbazole]; phenyl(dibenzofuranyl)bi[carbazole]; phenyl(dibenzothiophenyl)bi[carbazole]; phenyl(dibenzoselenophenyl)bi[carbazole]; phenyl(dibenzofuranyl)bi[carbazole]; phenyl(dibenzothiophenyl)bi[carbazole]; phenyl(dibenzoselenophenyl)bi[carbazole]; phenyl(benzofuropyridinyl)bi[carbazole]; phenyl(benzothienopyridinyl)bi[carbazole]; phenyl(benzselenopyridinyl)bi[carbazole]; (dimethylfluorenyl)(dibenzofuranyl)bi[carbazole]; (dimethylfluorenyl)(dibenzothiophenyl)bi[carbazole]; (dimethylfluorenyl)(dibenzoselenophenyl)bi[carbazole]; (dimethylfluorenyl)(dibenzofuranyl)bi[carbazole]; (dimethylfluorenyl)(dibenzothiophenyl)bi[carbazole]; (dimethylfluorenyl)(dibenzoselenophenyl)bi[carbazole]; bi[phenylcarbazole]; phenyl(biphenylyl)bi[carbazole]; phenyl(biphenylyl)bi[carbazole]; bi[(biphenylyl)carbazole]; bi[(biphenylyl)carbazole]; triphenylbi[carbazole]; bi[diphenylcarbazole]; phenyl[phenyl(biphenylyl)]bi[carbazole]; bi[phenylcarbazole]; phenyl(biphenylyl)bi[carbazole]; phenyl(biphenylyl)bi[carbazole]; bi[(biphenylyl)carbazole]; bi[(biphenylyl)carbazole]; bi[(biphenylyl)carbazole]; triphenylbi[carbazole]; bi[diphenylcarbazole]; phenyl[phenyl(biphenylyl)]bi[carbazole]; phenyl(terphenylyl)bi[carbazole]; phenyl(terphenylyl)bi[carbazole]; phenyl(terphenylyl)bi[carbazole]; phenyl(diphenylpyridinyl)bi[carbazole]

### (Group 21A)

(dibenzofuranyl)(diphenyltriazinyl)biphenyl; (dibenzothiophenyl)(diphenyltriazinyl)biphenyl; (dibenzoselenophenyl)(diphenyltriazinyl)biphenyl; (dibenzofuranyl)(diphenyltriazinyl)terbenzene; (dibenzothiophenyl)(diphenyltriazinyl)terbenzene; (dibenzoselenophenyl)(diphenyltriazinyl)terbenzene; (dibenzofuranyl)(diphenyltriazinyl)terbenzene; (dibenzothiophenyl)(diphenyltriazinyl)terbenzene; (dibenzoselenophenyl)(diphenyltriazinyl)terbenzene; (phenyldibenzofuranyl)(diphenyltriazinyl)biphenyl; (phenyldibenzothiophenyl)(diphenyltriazinyl)biphenyl; (phenyldibenzoselenophenyl)(diphenyltriazinyl)biphenyl; [(biphenylyl)dibenzofuranyl](diphenyltriazinyl)biphenyl; [(biphenylyl)dibenzothiophenyl](diphenyltriazinyl)biphenyl; [(biphenylyl)dibenzoselenophenyl](diphenyltriazinyl)biphenyl; [(biphenylyl)dibenzofuranyl](diphenyltriazinyl)biphenyl; [(biphenylyl)dibenzothiophenyl](diphenyltriazinyl)biphenyl; [(biphenylyl)dibenzoselenophenyl](diphenyltriazinyl)biphenyl; (phenyldibenzofuranyl)(diphenyltriazinyl)terbenzene; (phenyldibenzothiophenyl)(diphenyltriazinyl)terbenzene; (phenyldibenzoselenophenyl)(diphenyltriazinyl)terbenzene; (phenyldibenzofuranyl)(diphenyltriazinyl)terbenzene; (phenyldibenzothiophenyl)(diphenyltriazinyl)terbenzene; (phenyldibenzoselenophenyl)(diphenyltriazinyl)terbenzene; [(biphenylyl)dibenzofuranyl](diphenyltriazinyl)terbenzene; [(biphenylyl)dibenzothiophenyl](diphenyltriazinyl)terbenzene; [(biphenylyl)dibenzoselenophenyl](diphenyltriazinyl)terbenzene; (dibenzofuranyl)[phenyl(biphenylyl)triazinyl]biphenyl; (dibenzothiophenyl)[phenyl(biphenylyl)triazinyl]biphenyl; (dibenzoselenophenyl)[phenyl(biphenylyl)triazinyl]biphenyl; (dibenzofuranyl)[phenyl(biphenylyl)triazinyl]biphenyl; (dibenzothiophenyl)[phenyl(biphenylyl)triazinyl]biphenyl; (dibenzoselenophenyl)[phenyl(biphenylyl)triazinyl]biphenyl; (dibenzofuranyl)[bis(biphenylyl)triazinyl]biphenyl; (dibenzothiophenyl)[bis(biphenylyl)triazinyl]biphenyl; (dibenzoselenophenyl)[bis(biphenylyl)triazinyl]biphenyl; (dibenzofuranyl)[phenyl(dimethylfluorenyl)triazinyl]biphenyl; (dibenzothiophenyl)[phenyl(dimethylfluorenyl)triazinyl]biphenyl; (dibenzoselenophenyl)[phenyl(dimethylfluorenyl)triazinyl]biphenyl; (phenyldibenzofuranyl)[phenyl(biphenylyl)triazinyl]biphenyl; (phenyldibenzothiophenyl)[phenyl(biphenylyl)triazinyl]biphenyl; (phenyldibenzoselenophenyl)[phenyl(biphenylyl)triazinyl]biphenyl; (phenyldibenzofuranyl)[phenyl(biphenylyl)triazinyl]biphenyl; (phenyldibenzothiophenyl)[phenyl(biphenylyl)triazinyl]biphenyl; (phenyldibenzoselenophenyl)[phenyl(biphenylyl)triazinyl]biphenyl; (phenyldibenzofuranyl)[phenyl(dimethylfluorenyl)triazinyl]biphenyl; (phenyldibenzothiophenyl)[phenyl(dimethylfluorenyl)triazinyl]biphenyl; (phenyldibenzoselenophenyl)[phenyl(dimethylfluorenyl)triazinyl]biphenyl; (phenyldibenzofuranyl)[phenyl(biphenylyl)triazinyl]terbenzene; (phenyldibenzothiophenyl)[phenyl(biphenylyl)triazinyl]terbenzene; (phenyldibenzoselenophenyl)[phenyl(biphenylyl)triazinyl]terbenzene; (phenyldibenzofuranyl)[phenyl(biphenylyl)triazinyl]terbenzene; (phenyldibenzothiophenyl)[phenyl(biphenylyl)triazinyl]terbenzene; (phenyldibenzoselenophenyl)[phenyl(biphenylyl)triazinyl]terbenzene; (phenyldibenzofuranyl)[phenyl(dimethylfluorenyl)triazinyl]terbenzene; (phenyldibenzothiophenyl)[phenyl(dimethylfluorenyl)triazinyl]terbenzene; (phenyldibenzoselenophenyl)[phenyl(dimethylfluorenyl)triazinyl]terbenzene; [(biphenylyl)dibenzofuranyl][phenyl(biphenylyl)triazinyl]biphenyl; [(biphenylyl)dibenzothiophenyl][phenyl(biphenylyl)triazinyl]biphenyl; [(biphenylyl)dibenzoselenophenyl][phenyl(biphenylyl)triazinyl]biphenyl; [(biphenylyl)dibenzofuranyl][phenyl(biphenylyl)triazinyl]biphenyl; [(biphenylyl)dibenzothiophenyl][phenyl(biphenylyl)triazinyl]biphenyl; [(biphenylyl)dibenzoselenophenyl][phenyl(biphenylyl)triazinyl]biphenyl; [(biphenylyl)dibenzofuranyl][phenyl(dimethylfluorenyl)triazinyl]biphenyl; [(biphenylyl)dibenzothiophenyl][phenyl(dimethylfluorenyl)triazinyl]biphenyl; [(biphenylyl)dibenzoselenophenyl][phenyl(dimethylfluorenyl)triazinyl]biphenyl; (diphenyltriazinyl)(dibenzofuranyl)terbenzene; (diphenyltriazinyl)(dibenzothiophenyl)terbenzene; (diphenyltriazinyl)(dibenzoselenophenyl)terbenzene; (dibenzofuranyl)(diphenyltriazinyl)terbenzene; (dibenzothiophenyl)(diphenyltriazinyl)terbenzene; (dibenzoselenophenyl)(diphenyltriazinyl)terbenzene; (diphenyltriazinyl)(dibenzofuranyl)biphenyl; (diphenyltriazinyl)(dibenzothiophenyl)biphenyl; (diphenyltriazinyl)(dibenzoselenophenyl)biphenyl; (diphenyltriazinyl)(dibenzofuranyl)biphenyl; (diphenyltriazinyl)(dibenzothiophenyl)biphenyl; (diphenyltriazinyl)(dibenzoselenophenyl)biphenyl; (diphenyltriazinyl)(dibenzofuranyl)biphenyl; (diphenyltriazinyl)(dibenzothiophenyl)biphenyl; (diphenyltriazinyl)(dibenzoselenophenyl)biphenyl; (dibenzofuranyl)(diphenyltriazinyl)terbenzene; (dibenzothiophenyl)(diphenyltriazinyl)terbenzene; (dibenzoselenophenyl)(diphenyltriazinyl)terbenzene; (dibenzofuranyl)(diphenyltriazinyl)terbenzene; (dibenzothiophenyl)(diphenyltriazinyl)terbenzene; (dibenzoselenophenyl)(diphenyltriazinyl)terbenzene; (dibenzofuranyl)(diphenyltriazinyl)terbenzene; (dibenzothiophenyl)(diphenyltriazinyl)terbenzene; (dibenzoselenophenyl)(diphenyltriazinyl)terbenzene; (diphenyltriazinyl)(diphenyldibenzofuranyl)biphenyl; (diphenyltriazinyl)(diphenyldibenzothiophenyl)biphenyl; (diphenyltriazinyl)(diphenyldibenzoselenophenyl)biphenyl; (diphenyltriazinyl)(diphenyldibenzofuranyl)biphenyl; (diphenyltriazinyl)(diphenyldibenzothiophenyl)biphenyl; (diphenyltriazinyl)(diphenyldibenzoselenophenyl)biphenyl; (diphenyltriazinyl)(phenyldibenzofuranyl)biphenyl; (diphenyltriazinyl)(phenyldibenzothiophenyl)biphenyl; (diphenyltriazinyl)(phenyldibenzoselenophenyl)biphenyl; (diphenyltriazinyl)(phenyldibenzofuranyl)(pyridinyl)benzene; (diphenyltriazinyl)(phenyldibenzothiophenyl)(pyridinyl)benzene; (diphenyltriazinyl)(phenyldibenzoselenophenyl)(pyridinyl)benzene; (diphenyltriazinyl)(phenyldibenzofuranyl)(pyridinyl)benzene; (diphenyltriazinyl)(phenyldibenzothiophenyl)(pyridinyl)benzene; (diphenyltriazinyl)(phenyldibenzoselenophenyl)(pyridinyl)benzene; (diphenyltriazinyl)[(pyridinyl)dibenzofuranyl]biphenyl; (diphenyltriazinyl)[(pyridinyl)dibenzothiophenyl]biphenyl; (diphenyltriazinyl)[(pyridinyl)dibenzoselenophenyl]biphenyl; (diphenyltriazinyl)(dibenzofuranyl)terbenzene; (diphenyltriazinyl)(dibenzothiophenyl)terbenzene; (diphenyltriazinyl)(dibenzoselenophenyl)terbenzene; (dibenzofuranyl)(diphenyltriazinyl)terbenzene; (dibenzothiophenyl)(diphenyltriazinyl)terbenzene; (dibenzoselenophenyl)(diphenyltriazinyl)terbenzene; (diphenyltriazinyl)(dibenzofuranyl)(dimethylfluorenyl)benzene; (diphenyltriazinyl)(dibenzothiophenyl)(dimethylfluorenyl)benzene; (diphenyltriazinyl)(dibenzoselenophenyl)(dimethylfluorenyl)benzene; (diphenyltriazinyl)(dimethylfluorenyl)biphenyl; (diphenyltriazinyl)(spiro[cyclopentanefluorene]yl)biphenyl; (diphenyltriazinyl)(dimethylfluorenyl)terbenzene; (diphenyltriazinyl)(diphenylfluorenyl)biphenyl; [phenyl(biphenylyl)triazinyl](dimethylfluorenyl)biphenyl; [phenyl(dimethylfluorenyl)triazinyl](dimethylfluorenyl)terbenzene; (diphenyltriazinyl)[bis(dimethylfluorenyl)]benzene; (diphenyltriazinyl)(dimethylfluorenyl)terbenzene; phenyl(diphenyltriazinyl)dibenzofuran; phenyl(diphenyltriazinyl)dibenzothiophene; phenyl(diphenyltriazinyl)dibenzoselenophene; phenyl[phenyl(biphenylyl)triazinyl]dibenzofuran; phenyl[phenyl(biphenylyl)triazinyl]dibenzothiophene; phenyl[phenyl(biphenylyl)triazinyl]dibenzoselenophene; phenyl[phenyl(biphenylyl)triazinyl]dibenzofuran; phenyl[phenyl(biphenylyl)triazinyl]dibenzothiophene; phenyl[phenyl(biphenylyl)triazinyl]dibenzoselenophene; phenyl[bis(biphenylyl)triazinyl]dibenzofuran; phenyl[bis(biphenylyl)triazinyl]dibenzothiophene; phenyl[bis(biphenylyl)triazinyl]dibenzoselenophene; phenyl[bis(biphenylyl)triazinyl]dibenzofuran; phenyl[bis(biphenylyl)triazinyl]dibenzothiophene; phenyl[bis(biphenylyl)triazinyl]dibenzoselenophene; phenyl[bis(biphenylyl)triazinyl]dibenzofuran; phenyl[bis(biphenylyl)triazinyl]dibenzothiophene; phenyl[bis(biphenylyl)triazinyl]dibenzoselenophene; (diphenyltriazinyl)(phenyldibenzofuranyl)benzene; (diphenyltriazinyl)(phenyldibenzothiophenyl)benzene; (diphenyltriazinyl)(phenyldibenzoselenophenyl)benzene; [phenyl(biphenylyl)triazinyl](phenyldibenzofuranyl)benzene; [phenyl(biphenylyl)triazinyl](phenyldibenzothiophenyl)benzene; [phenyl(biphenylyl)triazinyl](phenyldibenzoselenophenyl)benzene; [phenyl(biphenylyl)triazinyl](phenyldibenzofuranyl)benzene; [phenyl(biphenylyl)triazinyl](phenyldibenzothiophenyl)benzene; [phenyl(biphenylyl)triazinyl](phenyldibenzoselenophenyl)benzene; (diphenyltriazinyl)(phenyldibenzofuranyl)benzene; (diphenyltriazinyl)(phenyldibenzothiophenyl)benzene; (diphenyltriazinyl)(phenyldibenzoselenophenyl)benzene; [phenyl(biphenylyl)triazinyl](phenyldibenzofuranyl)benzene; [phenyl(biphenylyl)triazinyl](phenyldibenzothiophenyl)benzene; [phenyl(biphenylyl)triazinyl](phenyldibenzoselenophenyl)benzene; [phenyl(biphenylyl)triazinyl](phenyldibenzofuranyl)benzene; [phenyl(biphenylyl)triazinyl](phenyldibenzothiophenyl)benzene; [phenyl(biphenylyl)triazinyl](phenyldibenzoselenophenyl)benzene; (diphenyltriazinyl)(phenyldibenzofuranyl)biphenyl; (diphenyltriazinyl)(phenyldibenzothiophenyl)biphenyl; (diphenyltriazinyl)(phenyldibenzoselenophenyl)biphenyl; [phenyl(biphenylyl)triazinyl](phenyldibenzofuranyl)biphenyl; [phenyl(biphenylyl)triazinyl](phenyldibenzothiophenyl)biphenyl; [phenyl(biphenylyl)triazinyl](phenyldibenzoselenophenyl)biphenyl; [phenyl(biphenylyl)triazinyl](phenyldibenzofuranyl)biphenyl; [phenyl(biphenylyl)triazinyl](phenyldibenzothiophenyl)biphenyl; [phenyl(biphenylyl)triazinyl](phenyldibenzoselenophenyl)biphenyl; (diphenyltriazinyl)(phenyldibenzofuranyl)biphenyl; (diphenyltriazinyl)(phenyldibenzothiophenyl)biphenyl; (diphenyltriazinyl)(phenyldibenzoselenophenyl)biphenyl; [phenyl(biphenylyl)triazinyl](phenyldibenzofuranyl)biphenyl; [phenyl(biphenylyl)triazinyl](phenyldibenzothiophenyl)biphenyl; [phenyl(biphenylyl)triazinyl](phenyldibenzoselenophenyl)biphenyl; [phenyl(biphenylyl)triazinyl](phenyldibenzofuranyl)biphenyl; [phenyl(biphenylyl)triazinyl](phenyldibenzothiophenyl)biphenyl; [phenyl(biphenylyl)triazinyl](phenyldibenzoselenophenyl)biphenyl; (diphenyltriazinyl)(phenyldibenzofuranyl)biphenyl; (diphenyltriazinyl)(phenyldibenzothiophenyl)biphenyl; (diphenyltriazinyl)(phenyldibenzoselenophenyl)biphenyl; [phenyl(biphenylyl)triazinyl](phenyldibenzofuranyl)biphenyl; [phenyl(biphenylyl)triazinyl](phenyldibenzothiophenyl)biphenyl; [phenyl(biphenylyl)triazinyl](phenyldibenzoselenophenyl)biphenyl; [phenyl(biphenylyl)triazinyl](phenyldibenzofuranyl)biphenyl; [phenyl(biphenylyl)triazinyl](phenyldibenzothiophenyl)biphenyl; [phenyl(biphenylyl)triazinyl](phenyldibenzoselenophenyl)biphenyl; (diphenyltriazinyl)(biphenylyl)dibenzofuran; (diphenyltriazinyl)(biphenylyl)dibenzothiophene; (diphenyltriazinyl)(biphenylyl)dibenzoselenophene; [phenyl(biphenylyl)triazinyl](biphenylyl)dibenzofuran; [phenyl(biphenylyl)triazinyl](biphenylyl)dibenzothiophene; [phenyl(biphenylyl)triazinyl](biphenylyl)dibenzoselenophene; [phenyl(biphenylyl)triazinyl](biphenylyl)dibenzofuran; [phenyl(biphenylyl)triazinyl](biphenylyl)dibenzothiophene; [phenyl(biphenylyl)triazinyl](biphenylyl)dibenzoselenophene; (diphenyltriazinyl)[(biphenylyl)dibenzofuranyl]benzene; (diphenyltriazinyl)[(biphenylyl)dibenzothiophenyl]benzene; (diphenyltriazinyl)[(biphenylyl)dibenzoselenophenyl]benzene; [phenyl(biphenylyl)triazinyl][(biphenylyl)dibenzofuranyl]benzene; [phenyl(biphenylyl)triazinyl][(biphenylyl)dibenzothiophenyl]benzene; [phenyl(biphenylyl)triazinyl][(biphenylyl)dibenzoselenophenyl]benzene; [phenyl(biphenylyl)triazinyl][(biphenylyl)dibenzofuranyl]benzene; [phenyl(biphenylyl)triazinyl][(biphenylyl)dibenzothiophenyl]benzene; [phenyl(biphenylyl)triazinyl][(biphenylyl)dibenzoselenophenyl]benzene; (diphenyltriazinyl)[(biphenylyl)dibenzofuranyl]benzene; (diphenyltriazinyl)[(biphenylyl)dibenzothiophenyl]benzene; (diphenyltriazinyl)[(biphenylyl)dibenzoselenophenyl]benzene; [phenyl(biphenylyl)triazinyl][(biphenylyl)dibenzofuranyl]benzene; [phenyl(biphenylyl)triazinyl][(biphenylyl)dibenzothiophenyl]benzene; [phenyl(biphenylyl)triazinyl][(biphenylyl)dibenzoselenophenyl]benzene; [phenyl(biphenylyl)triazinyl][(biphenylyl)dibenzofuranyl]benzene; [phenyl(biphenylyl)triazinyl][(biphenylyl)dibenzothiophenyl]benzene; [phenyl(biphenylyl)triazinyl][(biphenylyl)dibenzoselenophenyl]benzene; (diphenyltriazinyl)(biphenylyl)dibenzofuran; (diphenyltriazinyl)(biphenylyl)dibenzothiophene; (diphenyltriazinyl)(biphenylyl)dibenzoselenophene; [phenyl(biphenylyl)triazinyl](biphenylyl)dibenzofuran; [phenyl(biphenylyl)triazinyl](biphenylyl)dibenzothiophene; [phenyl(biphenylyl)triazinyl][(biphenylyl)dibenzoselenophene; [phenyl(biphenylyl)triazinyl](biphenylyl)dibenzofuran; [phenyl(biphenylyl)triazinyl](biphenylyl)dibenzothiophene; [phenyl(biphenylyl)triazinyl][(biphenylyl)dibenzoselenophene; (diphenyltriazinyl)[(biphenylyl)dibenzofuranyl]benzene; (diphenyltriazinyl)[(biphenylyl)dibenzothiophenyl]benzene; (diphenyltriazinyl)[(biphenylyl)dibenzoselenophenyl]benzene; [phenyl(biphenylyl)triazinyl][(biphenylyl)dibenzofuranyl]benzene; [phenyl(biphenylyl)triazinyl][(biphenylyl)dibenzothiophenyl]benzene; [phenyl(biphenylyl)triazinyl][(biphenylyl)dibenzoselenophenyl]benzene; [phenyl(biphenylyl)triazinyl][(biphenylyl)dibenzofuranyl]benzene; [phenyl(biphenylyl)triazinyl][(biphenylyl)dibenzothiophenyl]benzene; [phenyl(biphenylyl)triazinyl][(biphenylyl)dibenzoselenophenyl]benzene; (diphenyltriazinyl)[(biphenylyl)dibenzofuranyl]benzene; (diphenyltriazinyl)[(biphenylyl)dibenzothiophenyl]benzene; (diphenyltriazinyl)[(biphenylyl)dibenzoselenophenyl]benzene; [phenyl(biphenylyl)triazinyl][(biphenylyl)dibenzofuranyl]benzene; [phenyl(biphenylyl)triazinyl][(biphenylyl)dibenzothiophenyl]benzene; [phenyl(biphenylyl)triazinyl][(biphenylyl)dibenzoselenophenyl]benzene; [phenyl(biphenylyl)triazinyl][(biphenylyl)dibenzofuranyl]benzene; [phenyl(biphenylyl)triazinyl][(biphenylyl)dibenzothiophenyl]benzene; [phenyl(biphenylyl)triazinyl][(biphenylyl)dibenzoselenophenyl]benzene; phenyl[phenyl(dimethylfluorenyl)triazinyl]dibenzofuran; phenyl[phenyl(dimethylfluorenyl)triazinyl]dibenzothiophene; phenyl[phenyl(dimethylfluorenyl)triazinyl]dibenzoselenophene; phenyl[(biphenylyl)(dimethylfluorenyl)triazinyl]dibenzofuran; phenyl[(biphenylyl)(dimethylfluorenyl)triazinyl]dibenzothiophene; phenyl[(biphenylyl)(dimethylfluorenyl)triazinyl]dibenzoselenophene; phenyl[(biphenylyl)(dimethylfluorenyl)triazinyl]dibenzofuran; phenyl[(biphenylyl)(dimethylfluorenyl)triazinyl]dibenzothiophene; phenyl[(biphenylyl)(dimethylfluorenyl)triazinyl]dibenzoselenophene; [phenyl(dimethylfluorenyl)triazinyl](phenyldibenzofuranyl)benzene; [phenyl(dimethylfluorenyl)triazinyl](phenyldibenzothiophenyl)benzene; [phenyl(dimethylfluorenyl)triazinyl](phenyldibenzoselenophenyl)benzene; [bis(dimethylfluorenyl)triazinyl](phenyldibenzofuranyl)benzene; [bis(dimethylfluorenyl)triazinyl](phenyldibenzothiophenyl)benzene; [bis(dimethylfluorenyl)triazinyl](phenyldibenzoselenophenyl)benzene; (biphenylyl)[phenyl(dimethylfluorenyl)triazinyl]dibenzofuran; (biphenylyl)[phenyl(dimethylfluorenyl)triazinyl]dibenzothiophene; (biphenylyl)[phenyl(dimethylfluorenyl)triazinyl]dibenzoselenophene; (biphenylyl)[phenyl(dimethylfluorenyl)triazinyl]dibenzofuran; (biphenylyl)[phenyl(dimethylfluorenyl)triazinyl]dibenzothiophene; (biphenylyl)[phenyl(dimethylfluorenyl)triazinyl]dibenzoselenophene; [phenyl(dimethylfluorenyl)triazinyl][(biphenylyl)dibenzofuranyl]benzene; [phenyl(dimethylfluorenyl)triazinyl][(biphenylyl)dibenzothiophenyl]benzene; [phenyl(dimethylfluorenyl)triazinyl][(biphenylyl)dibenzoselenophenyl]benzene; [phenyl(dimethylfluorenyl)triazinyl][(biphenylyl)dibenzofuranyl]benzene; [phenyl(dimethylfluorenyl)triazinyl][(biphenylyl)dibenzothiophenyl]benzene; [phenyl(dimethylfluorenyl)triazinyl][(biphenylyl)dibenzoselenophenyl]benzene; phenyl[phenyl(phenyldimethylfluorenyl)triazinyl]dibenzofuran; phenyl[phenyl(phenyldimethylfluorenyl)triazinyl]dibenzothiophene; phenyl[phenyl(phenyldimethylfluorenyl)triazinyl]dibenzoselenophene; phenyl[phenyl(dimethylfluorenyl)triazinyl]dibenzofuran; phenyl[phenyl(dimethylfluorenyl)triazinyl]dibenzothiophene; phenyl[phenyl(dimethylfluorenyl)triazinyl]dibenzoselenophene; (biphenylyl)[phenyl(dimethylfluorenyl)triazinyl]dibenzofuran; (biphenylyl)[phenyl(dimethylfluorenyl)triazinyl]dibenzothiophene; (biphenylyl)[phenyl(dimethylfluorenyl)triazinyl]dibenzoselenophene; [phenyl(dimethylfluorenyl)triazinyl][(biphenylyl)dibenzofuranyl]benzene; [phenyl(dimethylfluorenyl)triazinyl][(biphenylyl)dibenzothiophenyl]benzene; [phenyl(dimethylfluorenyl)triazinyl][(biphenylyl)dibenzoselenophenyl]benzene; phenyl[phenyl(dimethylfluorenyl)triazinyl]dibenzofuran; phenyl[phenyl(dimethylfluorenyl)triazinyl]dibenzothiophene; phenyl[phenyl(dimethylfluorenyl)triazinyl]dibenzoselenophene; phenyl[phenyl(dimethylfluorenyl)triazinyl]dibenzofuran; phenyl[phenyl(dimethylfluorenyl)triazinyl]dibenzothiophene; phenyl[phenyl(dimethylfluorenyl)triazinyl]dibenzoselenophene; phenyl(diphenyltriazinyl)dibenzofuran; phenyl(diphenyltriazinyl)dibenzothiophene; phenyl(diphenyltriazinyl)dibenzoselenophene; (diphenyltriazinyl)(phenyldibenzofuranyl)benzene; (diphenyltriazinyl)(phenyldibenzothiophenyl)benzene; (diphenyltriazinyl)(phenyldibenzoselenophenyl)benzene; (diphenyltriazinyl)[(biphenylyl)dibenzofuranyl]benzene; (diphenyltriazinyl)[(biphenylyl)dibenzothiophenyl]benzene; (diphenyltriazinyl)[(biphenylyl)dibenzoselenophenyl]benzene; (diphenyltriazinyl)(diphenyldibenzofuranyl)benzene; (diphenyltriazinyl)(diphenyldibenzothiophenyl)benzene; (diphenyltriazinyl)(diphenyldibenzoselenophenyl)benzene; phenyl(diphenyltriazinyl)dibenzofuran; phenyl(diphenyltriazinyl)dibenzothiophene; phenyl(diphenyltriazinyl)dibenzoselenophene; (biphenylyl)(diphenyltriazinyl)dibenzofuran; (biphenylyl)(diphenyltriazinyl)dibenzothiophene; (biphenylyl)(diphenyltriazinyl)dibenzoselenophene; (biphenylyl)(diphenyltriazinyl)dibenzofuran; (biphenylyl)(diphenyltriazinyl)dibenzothiophene; (biphenylyl)(diphenyltriazinyl)dibenzoselenophene; (diphenyltriazinyl)(diphenyldibenzofuranyl)benzene; (diphenyltriazinyl)(diphenyldibenzothiophenyl)benzene; (diphenyltriazinyl)(diphenyldibenzoselenophenyl)benzene; (diphenyltriazinyl)[(biphenylyl)dibenzofuranyl]benzene; (diphenyltriazinyl)[(biphenylyl)dibenzothiophenyl]benzene; (diphenyltriazinyl)[(biphenylyl)dibenzoselenophenyl]benzene; phenyl(diphenyltriazinyl)dibenzofuran; phenyl(diphenyltriazinyl)dibenzothiophene; phenyl(diphenyltriazinyl)dibenzoselenophene; (diphenyltriazinyl)(phenyldibenzofuranyl)benzene; (diphenyltriazinyl)(phenyldibenzothiophenyl)benzene; (diphenyltriazinyl)(phenyldibenzoselenophenyl)benzene; (diphenyltriazinyl)(phenyldibenzofuranyl)benzene; (diphenyltriazinyl)(phenyldibenzothiophenyl)benzene; (diphenyltriazinyl)(phenyldibenzoselenophenyl)benzene; (diphenyltriazinyl)[(biphenylyl)dibenzofuranyl]benzene; (diphenyltriazinyl)[(biphenylyl)dibenzothiophenyl]benzene; (diphenyltriazinyl)[(biphenylyl)dibenzoselenophenyl]benzene; (diphenyltriazinyl)[(biphenylyl)dibenzofuranyl]benzene; (diphenyltriazinyl)[(biphenylyl)dibenzothiophenyl]benzene; (diphenyltriazinyl)[(biphenylyl)dibenzoselenophenyl]benzene; (diphenyltriazinyl)(phenyldibenzofuranyl)benzene; (diphenyltriazinyl)(phenyldibenzothiophenyl)benzene; (diphenyltriazinyl)(phenyldibenzoselenophenyl)benzene; (diphenyltriazinyl)(phenyldibenzofuranyl)pyridine; (diphenyltriazinyl)(phenyldibenzothiophenyl)pyridine; (diphenyltriazinyl)(phenyldibenzoselenophenyl)pyridine; [phenyl(phenylpyridinyl)triazinyl](phenyldibenzofuranyl)pyridine; [phenyl(phenylpyridinyl)triazinyl](phenyldibenzothiophenyl)pyridine; [phenyl(phenylpyridinyl)triazinyl](phenyldibenzoselenophenyl)pyridine; [phenyl(phenylpyridinyl)triazinyl](phenyldibenzofuranyl)benzene; [phenyl(phenylpyridinyl)triazinyl](phenyldibenzothiophenyl)benzene; [phenyl(phenylpyridinyl)triazinyl](phenyldibenzoselenophenyl)benzene; (diphenyltriazinyl)[(biphenylyl)dibenzofuranyl]pyridine; (diphenyltriazinyl)[(biphenylyl)dibenzothiophenyl]pyridine; (diphenyltriazinyl)[(biphenylyl)dibenzoselenophenyl]pyridine; phenyl[phenyl(dimethylbenzofuropyridine)triazinyl]dibenzofuran; phenyl[phenyl(dimethylbenzothienopyridine)triazinyl]dibenzothiophene; phenyl[phenyl(dimethylbenzselenopyridine)triazinyl]dibenzoselenophene; [(biphenylyl)(dimethylbenzofuropyridine)triazinyl]dibenzofuran; [(biphenylyl)(dimethylbenzothienopyridine)triazinyl]dibenzothiophene; [(biphenylyl)(dimethylbenzselenopyridine)triazinyl]dibenzoselenophene; phenyl[phenyl(dimethylbenzofuropyridine)triazinyl]dibenzofuran; phenyl[phenyl(dimethylbenzothienopyridine)triazinyl]dibenzothiophene; phenyl[phenyl(dimethylbenzselenopyridine)triazinyl]dibenzoselenophene; phenyl[phenyl(spiro[cyclohexanefluorene]yl)triazinyl]dibenzofuran; phenyl[phenyl(spiro[cyclohexanefluorene]yl)triazinyl]dibenzothiophene; phenyl[phenyl(spiro[cyclohexanefluorene]yl)triazinyl]dibenzoselenophene; phenyl[phenyl(spirobi[fluorene]yl)triazinyl]dibenzofuran; phenyl[phenyl(spirobi[fluorene]yl)triazinyl]dibenzothiophene; phenyl[phenyl(spirobi[fluorene]yl)triazinyl]dibenzoselenophene; benzene-d₃[(diphenyl-d₁₀)triazinyl][(phenyl-d₅)dibenzofuranyl-d₆]; benzene-d₃[(diphenyl-d₁₀)triazinyl][(phenyl-d₅)dibenzothiophenyl-d₆]; benzene-d₃[(diphenyl-d₁₀)triazinyl][(phenyl-d₅)dibenzoselenophenyl-d₆]; benzene-d₃[(diphenyl-d₁₀)triazinyl][(biphenylyl-d₉)dibenzofuranyl-d₆]; benzene-d₃[(diphenyl-d₁₀)triazinyl][(biphenylyl-do)dibenzothiophenyl-d₆]; benzene-d₃[(diphenyl-d₁₀)triazinyl][(biphenylyl-d₉)dibenzoselenophenyl-d₆]; phenyl{phenyl[(dimethyl-d₆)fluorenyl]triazinyl}dibenzofuran; phenyl{phenyl[(dimethyl-d₆)fluorenyl]triazinyl}dibenzothiophene; phenyl{phenyl[(dimethyl-d₆)fluorenyl]triazinyl}dibenzoselenophene; benzene-d₃[(diphenyl-d₁₀)triazinyl][(biphenylyl-d₉)dibenzofuranyl-d₆]; benzene-d₃[(diphenyl-d₁₀)triazinyl][(biphenylyl-d₉)dibenzothiophenyl-d₆]; benzene-d₃[(diphenyl-d₁₀)triazinyl][(biphenylyl-d₉)dibenzoselenophenyl-d₆]; [(diphenyl-d₁₀)triazinyl][(biphenylyl)dibenzofuranyl]benzene; [(diphenyl-d₁₀)triazinyl][(biphenylyl)dibenzothiophenyl]benzene; [(diphenyl-d₁₀)triazinyl][(biphenylyl)dibenzoselenophenyl]benzene; (diphenyltriazinyl)[(biphenylyl)dibenzofuranyl]biphenyl; (diphenyltriazinyl)[(biphenylyl)dibenzothiophenyl]biphenyl; (diphenyltriazinyl)[(biphenylyl)dibenzoselenophenyl]biphenyl; (difluorophenyltriazinyl)[(biphenylyl)dibenzofuranyl]benzene; (difluorophenyltriazinyl)[(biphenylyl)dibenzothiophenyl]benzene; (difluorophenyltriazinyl)[(biphenylyl)dibenzoselenophenyl]benzene; (diphenyltriazinyl)[(dimethylfluorenyl)dibenzofuranyl]benzene; (diphenyltriazinyl)[(dimethylfluorenyl)dibenzothiophenyl]benzene; (diphenyltriazinyl)[(dimethylfluorenyl)dibenzoselenophenyl]benzene; (diphenyltriazinyl)[(dimethylfluorenyl)dibenzofuranyl]biphenyl; (diphenyltriazinyl)[(dimethylfluorenyl)dibenzothiophenyl]biphenyl; (diphenyltriazinyl)[(dimethylfluorenyl)dibenzoselenophenyl]biphenyl; (diphenyltriazinyl)[(dimethylfluorenyl)dibenzofuranyl]biphenyl; (diphenyltriazinyl)[(dimethylfluorenyl)dibenzothiophenyl]biphenyl; (diphenyltriazinyl)[(dimethylfluorenyl)dibenzoselenophenyl]biphenyl; (diphenyltriazinyl)[(dimethylfluorenyl)dibenzofuranyl]biphenyl; (diphenyltriazinyl)[(dimethylfluorenyl)dibenzothiophenyl]biphenyl; (diphenyltriazinyl)[(dimethylfluorenyl)dibenzoselenophenyl]biphenyl; (diphenyltriazinyl)[(dimethylfluorenyl)dibenzofuranyl]pyridine; (diphenyltriazinyl)[(dimethylfluorenyl)dibenzothiophenyl]pyridine; (diphenyltriazinyl)[(dimethylfluorenyl)dibenzoselenophenyl]pyridine; (diphenyltriazinyl)[(dimethylfluorenyl)dibenzofuranyl]benzene; (diphenyltriazinyl)[(dimethylfluorenyl)dibenzothiophenyl]benzene; (diphenyltriazinyl)[(dimethylfluorenyl)dibenzoselenophenyl]benzene; (diphenyltriazinyl)[(dimethylfluorenyl)dibenzofuranyl]benzene; (diphenyltriazinyl)[(dimethylfluorenyl)dibenzothiophenyl]benzene; (diphenyltriazinyl)[(dimethylfluorenyl)dibenzoselenophenyl]benzene; (diphenyltriazinyl)[(dimethylfluorenyl)dibenzofuranyl]benzene; (diphenyltriazinyl)[(dimethylfluorenyl)dibenzothiophenyl]benzene; (diphenyltriazinyl)[(dimethylfluorenyl)dibenzoselenophenyl]benzene; (diphenyltriazinyl)[(dimethylfluorenyl)dibenzofuranyl]biphenyl; (diphenyltriazinyl)[(dimethylfluorenyl)dibenzothiophenyl]biphenyl; (diphenyltriazinyl)[(dimethylfluorenyl)dibenzoselenophenyl]biphenyl; [phenyl(biphenylyl)triazinyl][(dimethylfluorenyl)dibenzofuranyl]benzene; [phenyl(biphenylyl)triazinyl][(dimethylfluorenyl)dibenzothiophenyl]benzene; [phenyl(biphenylyl)triazinyl][(dimethylfluorenyl)dibenzoselenophenyl]benzene; [phenyl(biphenylyl)triazinyl][(dimethylfluorenyl)dibenzofuranyl]benzene; [phenyl(biphenylyl)triazinyl][(dimethylfluorenyl)dibenzothiophenyl]benzene; [phenyl(biphenylyl)triazinyl][(dimethylfluorenyl)dibenzoselenophenyl]benzene; [phenyl(dimethylfluorenyl)triazinyl][(dimethylfluorenyl)dibenzofuranyl]benzene; [phenyl(dimethylfluorenyl)triazinyl][(dimethylfluorenyl)dibenzothiophenyl]benzene; [phenyl(dimethylfluorenyl)triazinyl][(dimethylfluorenyl)dibenzoselenophenyl]benzene; (diphenyltriazinyl)[(dimethylfluorenyl)dibenzofuranyl]benzene; (diphenyltriazinyl)[(dimethylfluorenyl)dibenzothiophenyl]benzene; (diphenyltriazinyl)[(dimethylfluorenyl)dibenzoselenophenyl]benzene; (diphenyltriazinyl)[bis(dimethylfluorenyl)dibenzofuranyl]benzene; (diphenyltriazinyl)[bis(dimethylfluorenyl)dibenzothiophenyl]benzene; (diphenyltriazinyl)[bis(dimethylfluorenyl)dibenzoselenophenyl]benzene; [phenyl(terphenylyl)triazinyl][(dimethylfluorenyl)dibenzofuranyl]benzene; [phenyl(terphenylyl)triazinyl][(dimethylfluorenyl)dibenzothiophenyl]benzene; [phenyl(terphenylyl)triazinyl][(dimethylfluorenyl)dibenzoselenophenyl]benzene; (diphenyltriazinyl)[(spiro[cyclopentanefluorene]yl)dibenzofuranyl]benzene; (diphenyltriazinyl)[(spiro[cyclopentanefluorene]yl)dibenzothiophenyl]benzene; (diphenyltriazinyl)[(spiro[cyclopentanefluorene]yl)dibenzoselenophenyl]benzene; (diphenyltriazinyl)[(diphenylfluorenyl)dibenzofuranyl]benzene; (diphenyltriazinyl)[(diphenylfluorenyl)dibenzothiophenyl]benzene; (diphenyltriazinyl)[(diphenylfluorenyl)dibenzoselenophenyl]benzene; (diphenyltriazinyl)[(spirobi[fluorene]yl)dibenzofuranyl]benzene; (diphenyltriazinyl)[(spirobi[fluorene]yl)dibenzothiophenyl]benzene; (diphenyltriazinyl)[(spirobi[fluorene]yl)dibenzoselenophenyl]benzene; (diphenyltriazinyl)[phenyl(dimethylfluorenyl)dibenzofuranyl]benzene; (diphenyltriazinyl)[phenyl(dimethylfluorenyl)dibenzothiophenyl]benzene; (diphenyltriazinyl)[phenyl(dimethylfluorenyl)dibenzoselenophenyl]benzene; (diphenyltriazinyl)[phenyl(dimethylfluorenyl)dibenzofuranyl]benzene; (diphenyltriazinyl)[phenyl(dimethylfluorenyl)dibenzothiophenyl]benzene; (diphenyltriazinyl)[phenyl(dimethylfluorenyl)dibenzoselenophenyl]benzene; (diphenyltriazinyl)[(phenyldimethylfluorenyl)dibenzofuranyl]benzene; (diphenyltriazinyl)[(phenyldimethylfluorenyl)dibenzothiophenyl]benzene; (diphenyltriazinyl)[(phenyldimethylfluorenyl)dibenzoselenophenyl]benzene; (diphenyltriazinyl){[(dimethyl-d₆)fluorenyl]dibenzofuranyl}benzene; (diphenyltriazinyl){[(dimethyl-d₆)fluorenyl]dibenzothiophenyl}benzene; (diphenyltriazinyl){[(dimethyl-d₆)fluorenyl]dibenzoselenophenyl}benzene; (diphenyltriazinyl)[(dimethylfluorenyl)dibenzofuranyl]benzene; (diphenyltriazinyl)[(dimethylfluorenyl)dibenzothiophenyl]benzene; (diphenyltriazinyl)[(dimethylfluorenyl)dibenzoselenophenyl]benzene; (diphenyltriazinyl)[(dimethylfluorenyl)dibenzofuranyl]benzene; (diphenyltriazinyl)[(dimethylfluorenyl)dibenzothiophenyl]benzene; (diphenyltriazinyl)[(dimethylfluorenyl)dibenzoselenophenyl]benzene; (diphenyltriazinyl)[(dimethylfluorenyl)dibenzofuranyl]benzene; (diphenyltriazinyl)[(dimethylfluorenyl)dibenzothiophenyl]benzene; (diphenyltriazinyl)[(dimethylfluorenyl)dibenzoselenophenyl]benzene; (diphenyltriazinyl)[(dimethylfluorenyl)dibenzofuranyl]benzene; (diphenyltriazinyl)[(dimethylfluorenyl)dibenzothiophenyl]benzene; (diphenyltriazinyl)[(dimethylfluorenyl)dibenzoselenophenyl]benzene; (diphenyltriazinyl)[phenyl(dimethylfluorenyl)dibenzofuranyl]benzene; (diphenyltriazinyl)[phenyl(dimethylfluorenyl)dibenzothiophenyl]benzene; (diphenyltriazinyl)[phenyl(dimethylfluorenyl)dibenzoselenophenyl]benzene; (diphenyltriazinyl)[phenyl(dimethylfluorenyl)dibenzofuranyl]benzene; (diphenyltriazinyl)[phenyl(dimethylfluorenyl)dibenzothiophenyl]benzene; (diphenyltriazinyl)[phenyl(dimethylfluorenyl)dibenzoselenophenyl]benzene; (diphenyltriazinyl)[(dimethylfluorenyl)dibenzofuranyl]benzene; (diphenyltriazinyl)[(dimethylfluorenyl)dibenzothiophenyl]benzene; (diphenyltriazinyl)[(dimethylfluorenyl)dibenzoselenophenyl]benzene; [(diphenyl-d₁₀)triazinyl][(dimethylfluorenyl)dibenzofuranyl]benzene; [(diphenyl-d₁₀)triazinyl][(dimethylfluorenyl)dibenzothiophenyl]benzene; [(diphenyl-d₁₀)triazinyl][(dimethylfluorenyl)dibenzoselenophenyl]benzene; benzene-d₄[(diphenyl-d₁₀)triazinyl][(dimethylfluorenyl)dibenzofuranyl]; benzene-d₄[(diphenyl-d₁₀)triazinyl][(dimethylfluorenyl)dibenzothiophenyl]; benzene-d₄[(diphenyl-d₁₀)triazinyl][(dimethylfluorenyl)dibenzoselenophenyl]; [(diphenyl-d₁₀)triazinyl]{[(dimethyl-d₆)fluorenyl]dibenzofuranyl}benzene; [(diphenyl-d₁₀)triazinyl]{[(dimethyl-d₆)fluorenyl]dibenzothiophenyl}benzene; [(diphenyl-d₁₀)triazinyl]{[(dimethyl-d₆)fluorenyl]dibenzoselenophenyl}benzene; benzene-d₄[(diphenyl-d₁₀)triazinyl][(dimethylfluorenyl)dibenzofuranyl]; benzene-d₄[(diphenyl-d₁₀)triazinyl][(dimethylfluorenyl)dibenzothiophenyl]; benzene-d₄[(diphenyl-d₁₀)triazinyl][(dimethylfluorenyl)dibenzoselenophenyl]; [bis(carbazole)yl]benzofuropyridine; [bis(carbazole)yl]benzothienopyridine; [bis(carbazole)yl]benzselenopyridine; [bis(carbazole)yl]benzofuropyridine; [bis(carbazole)yl]benzothienopyridine; [bis(carbazole)yl]benzselenopyridine; [bis(carbazole)yl]benzofuropyridine; [bis(carbazole)yl]benzothienopyridine; [bis(carbazole)yl]benzselenopyridine; [bis(carbazole)yl]benzofuropyridine; [bis(carbazole)yl]benzothienopyridine; [bis(carbazole)yl]benzselenopyridine; (triphenylenyl)(carbazolyl)benzofuropyridine; (triphenylenyl)(carbazolyl)benzothienopyridine; (triphenylenyl)(carbazolyl)benzselenopyridine; (dibenzothiophenyl)(carbazolyl)benzofuropyridine; (dibenzothiophenyl)(carbazolyl)benzothienopyridine; (dibenzothiophenyl)(carbazolyl)benzselenopyridine; phenyl(triphenylenyl)benzofuropyridine; phenyl(triphenylenyl)benzothienopyridine; phenyl(triphenylenyl)benzselenopyridine; phenyl(triphenylenyl)benzofuropyridine; phenyl(triphenylenyl)benzothienopyridine; phenyl(triphenylenyl)benzselenopyridine; phenyl(triphenylenyl)bi[carbazole]; phenyl(triphenylenyl)bi[carbazole]; phenyl(triphenylenyl)bi[carbazole]; phenyl(dimethylfluorenyl)bi[carbazole]; phenyl(dimethylfluorenyl)bi[carbazole]; phenyl(dimethylfluorenyl)bi[carbazole]; phenyl(dimethylfluorenyl)bi[carbazole]; phenyl(dimethylfluorenyl)bi[carbazole]; phenyl(dimethylfluorenyl)bi[carbazole]; (biphenylyl)(dimethylfluorenyl)bi[carbazole]; (biphenylyl)(dimethylfluorenyl)bi[carbazole]; (terphenylyl)(dimethylfluorenyl)bi[carbazole]; diphenyl(ter[carbazole]); diphenyl(ter[carbazole]); diphenyl(ter[carbazole]); bi(phenylcarbazole); phenyl(biphenylyl)bi[carbazole]; phenyl(biphenylyl)bi[carbazole]; bi[(biphenylyl)carbazole]; bi[(biphenylyl)carbazole]; triphenylbi[carbazole]; bi[diphenylcarbazole]; diphenyl(biphenylyl)bi[carbazole]:bi[phenylcarbazole]; phenyl(biphenylyl)bi[carbazole]; phenyl(biphenylyl)bi[carbazole]; bi[(biphenylyl)carbazole]; bi[(biphenylyl)carbazole]; triphenylbi[carbazole]; bi[diphenylcarbazole]; diphenyl(biphenylyl)bi[carbazole]: phenyl(terphenylyl)bi[carbazole]; phenyl(terphenylyl)bi[carbazole]; phenyl(terphenylyl)bi[carbazole]; phenyl(diphenylpyridinyl)bi[carbazole]; phenyl(dibenzofuranyl)bi[carbazole]; phenyl(dibenzothiophenyl)bi[carbazole]; phenyl(dibenzoselenophenyl)bi[carbazole]; phenyl(dibenzofuranyl)bi[carbazole]; phenyl(dibenzothiophenyl)bi[carbazole]; phenyl(dibenzoselenophenyl)bi[carbazole]; phenyl(dibenzofuranyl)bi[carbazole]; phenyl(dibenzothiophenyl)bi[carbazole]; phenyl(dibenzoselenophenyl)bi[carbazole]; phenyl(dibenzofuranyl)bi[carbazole]; phenyl(dibenzothiophenyl)bi[carbazole]; phenyl(dibenzoselenophenyl)bi[carbazole]; phenyl(dibenzofuranyl)bi[carbazole]; phenyl(dibenzothiophenyl)bi[carbazole]; phenyl(dibenzoselenophenyl)bi[carbazole]; (phenylcarbazolyl)(dibenzofuranyl)fluorene; (phenylcarbazolyl)(dibenzothiophenyl)fluorene; (phenylcarbazolyl)(dibenzoselenophenyl)fluorene; phenyl(dibenzofuranyl)bi[carbazole]; phenyl(dibenzothiophenyl)bi[carbazole]; phenyl(dibenzoselenophenyl)bi[carbazole]; phenyl(dibenzofuranyl)bi[carbazole]; phenyl(dibenzothiophenyl)bi[carbazole]; phenyl(dibenzoselenophenyl)bi[carbazole]; (biphenylyl)(dibenzofuranyl)bi[carbazole]; (biphenylyl)(dibenzothiophenyl)bi[carbazole]; (biphenylyl)(dibenzoselenophenyl)bi[carbazole]; (biphenylyl)(dibenzofuranyl)bi[carbazole]; (biphenylyl)(dibenzothiophenyl)bi[carbazole]; (biphenylyl)(dibenzoselenophenyl)bi[carbazole]; (biphenylyl)(dibenzofuranyl)bi[carbazole]; (biphenylyl)(dibenzothiophenyl)bi[carbazole]; (biphenylyl)(dibenzoselenophenyl)bi[carbazole]; phenyl(phenyldibenzofuranyl)bi[carbazole]; phenyl(phenyldibenzothiophenyl)bi[carbazole]; phenyl(phenyldibenzoselenophenyl)bi[carbazole]; phenyl(dibenzofuranyl)bi[carbazole]; phenyl(dibenzothiophenyl)bi[carbazole]; phenyl(dibenzoselenophenyl)bi[carbazole]; phenyl(dibenzofuranyl)bi[carbazole]; phenyl(dibenzothiophenyl)bi[carbazole]; phenyl(dibenzoselenophenyl)bi[carbazole]; phenyl(dibenzofuranyl)bi[carbazole]; phenyl(dibenzothiophenyl)bi[carbazole]; phenyl(dibenzoselenophenyl)bi[carbazole]; phenyl(dibenzofuranyl)bi[carbazole]; phenyl(dibenzothiophenyl)bi[carbazole]; phenyl(dibenzoselenophenyl)bi[carbazole]; phenyl(dibenzofuranyl)bi[carbazole]; phenyl(dibenzothiophenyl)bi[carbazole]; phenyl(dibenzoselenophenyl)bi[carbazole]; (naphthalenyl)(dibenzofuranyl)bi[carbazole]; (naphthalenyl)(dibenzothiophenyl)bi[carbazole]; (naphthalenyl)(dibenzoselenophenyl)bi[carbazole]; (dimethylfluorenyl)(dibenzofuranyl)bi[carbazole]; (dimethylfluorenyl)(dibenzothiophenyl)bi[carbazole]; (dimethylfluorenyl)(dibenzoselenophenyl)bi[carbazole]; (dimethylfluorenyl)(dibenzofuranyl)bi[carbazole]; (dimethylfluorenyl)(dibenzothiophenyl)bi[carbazole]; (dimethylfluorenyl)(dibenzoselenophenyl)bi[carbazole]; (triphenylenyl)dibenzofuran; (triphenylenyl)dibenzothiophene; (triphenylenyl)dibenzoselenophene; (triphenylenyl)(dibenzofuranyl)benzene; (triphenylenyl)(dibenzothiophenyl)benzene; (triphenylenyl)(dibenzoselenophenyl)benzene; (triphenylenyl)(phenyldibenzofuranyl)benzene; (triphenylenyl)(phenyldibenzothiophenyl)benzene; (triphenylenyl)(phenyldibenzoselenophenyl)benzene; (triphenylenyl)(dibenzofuranyl)biphenyl; (triphenylenyl)(dibenzothiophenyl)biphenyl; (triphenylenyl)(dibenzoselenophenyl)biphenyl; phenyl(triphenylenyl)dibenzofuran; phenyl(triphenylenyl)dibenzothiophene; phenyl(triphenylenyl)dibenzoselenophene; (biphenylyl)(triphenylenyl)dibenzofuran; (biphenylyl)(triphenylenyl)dibenzothiophene; (biphenylyl)(triphenylenyl)dibenzoselenophene; (terphenylyl)(triphenylenyl)dibenzofuran; (terphenylyl)(triphenylenyl)dibenzothiophene; (terphenylyl)(triphenylenyl)dibenzoselenophene; (biphenylyl)[(triphenylenyl)phenyl]dibenzofuran; (biphenylyl)[(triphenylenyl)phenyl]dibenzothiophene; (biphenylyl)[(triphenylenyl)phenyl]dibenzoselenophene; (carbazolyl)(triphenylenyl)dibenzofuran; (carbazolyl)(triphenylenyl)dibenzothiophene; (carbazolyl)(triphenylenyl)dibenzoselenophene; (carbazolyl)(triphenylenyl)biphenyl; (carbazolyl)(triphenylenyl)biphenyl; iridium(III)tris[phenylpyridine-N,C′]; iridium(III)tris[methyl(phenyl)pyridine-N,C′]; iridium(III)tris[(methyl-d₃)(phenyl)pyridine-N,C′]; iridium(III)tris[(dimethyl-d₆)(phenyl)pyridine-N,C′]; iridium(III)tris[(methyl-d₆)(diphenyl)pyridine-N,C′]; iridium(III)tris[(pyridinyl)biphenyl-N,C′]; iridium(III)tris[(methylpyridinyl)biphenyl-N,C′]; iridium(III)bis(phenylpyridine-N,C′[(pyridinyl)biphenyl-N,C′]; iridium(III) bis(phenylpyridine-N,C′[(pyridinyl)biphenyl-N,C′]; iridium(III)bis(phenylpyridine-N,C′[(methylpyridinyl)biphenyl-N,C′]; iridium(III)bis(phenylpyridine-N,C′[(methylpyridinyl)biphenyl-N,C′]; iridium(III)bis(phenylpyridine-N,C′{[(methyl-d₃)pyridinyl]biphenyl-N,C″}; iridium(III)bis(phenylpyridine-N,C′[(methyl)(diphenyl)pyridine-N,C′]; iridium(III)bis(phenylpyridine-N,C′[(methyl-d₃)(diphenyl)pyridine-N,C′]; iridium(III)bis(phenylpyridine-N,C′[ethane-d₃-(diphenyl)pyridine-N,C′]; iridium(III)bis(phenylpyridine-N,C′[(methyl)(diphenyl)pyridine-N,C′]; iridium(III)bis[(methyl)(phenyl)pyridine-N,C′][(methyl)(phenyl)(naphthalenyl)pyridine-N,C′]; iridium(III)bis[(methyl)(phenyl)pyridine-N,C′][(methyl)(diphenyl)pyridine-N,C′]; iridium(III)bis[(methyl)(phenyl)pyridine-N,C′][diphenylpyridine-N,C′]; iridium(III)bis{[(methylphenyl-d₃)pyridine-d₄]—N,C″}{[(diphenyl-d₉)pyridine-d₄]-N,C′}; iridium(III)bis[(methyl)(phenyl)pyridine-N,C′][diphenylpyridine-N,C′]; iridium(III)bis[(methyl-d₃)(phenyl)pyridine-N,C′][diphenylpyridine-N,C′]; iridium(III)bis[phenylpyridine-N,C′][(methyl)(diphenyl)pyridine-N,C′]; iridium(III)bis[phenylpyridine-N,C′][(methyl-d₃)(diphenyl)pyridine-N,C′]; iridium(III)bis[phenylpyridine-N,C′][(ethyl)(diphenyl)pyridine-N,C′]; iridium(III)bis[phenylpyridine-N,C′][(methyl)(phenyl)(fluorophenyl)pyridine-N,C′]; iridium(III)bis[phenylpyridine-N,C′][(dibenzofuranyl)pyridine-N,C′]; iridium(III)bis[phenylpyridine-N,C′][(isopropyl)(dibenzofuranyl)pyridine-N,C′]; iridium(III)bis[(methyl)(phenyl)pyridine-N,C′][(isopropyl)(dibenzofuranyl)pyridine-N,C′]; iridium(III)bis[phenylpyridine-N,C′][(isopropyl-d₇)(dibenzofuranyl)pyridine-N,C′]; iridium(III)bis[(methyl-d₃)(phenyl)pyridine-N,C′][(isopropyl-d₇)(dibenzofuranyl)pyridine-N,C′]; iridium(III)bis[(methyl-d₃)(phenyl)pyridine-N,C′][(methyl-d₃)(diphenyl)pyridine-N,C′]; iridium(III)bis[phenylpyridine-N,C′][(isopropyl)(isopropylbenzofuropyridinyl)pyridine-N,C′]; iridium(III)bis[phenylpyridine-N,C′][(benzofuropyridinyl)pyridine-N,C′]; iridium(III)bis[phenylpyridine-N,C′][(benzofuropyridinyl)pyridine-N,C′]; iridium(III)bis[phenylpyridine-N,C′][(methylbenzofuropyridinyl)pyridine-N,C′]; iridium(III)bis[phenylpyridine-N,C′]{[(methyl-d₃)benzofuropyridinyl]pyridine-N,C′}; iridium(III)bis[phenylpyridine-N,C′][(methylbenzofuropyridinyl)pyridine-N,C′]; iridium(III)bis[phenylpyridine-N,C′]{[(methyl-d₃)benzofuropyridinyl]pyridine-N,C′}; iridium(III)bis[(methyl-d₃)(phenyl)pyridine-N,C′]{[(methyl-d₃)benzofuropyridinyl]pyridine-N,C″}; iridium(III)bis[(methyl-d₃)(phenyl)pyridine-N,C′][(isopropyl-d₇)(benzofuropyridinyl)pyridine-N,C′]; iridium(III)bis[(methyl-d₃)(phenyl)pyridine-N,C′]{(isopropyl-d₇)[(methyl-d₃)benzofuropyridinyl]pyridine-N,C′}; iridium(III)bis[(isopropyl-d₇)(phenyl)pyridine-N,C′]{(isopropyl-d₇)[(methyl-d₃)benzofuropyridinyl]pyridine-N,C′}; iridium(III)bis[(methyl-d₃)(phenyl)pyridine-N,C′][(isopropyl-d₁)(benzofuropyridinyl)pyridine-N,C′]; iridium(III)bis[(methyl-d₃)(phenyl)pyridine-N,C′]{(isopropyl-d₁)[(methyl-d₃)benzofuropyridinyl]pyridine-N,C″}; iridium(III)bis[(methyl-d₃)(phenyl)pyridine-N,C′][(isopropyl)(methyl-d₃)(benzofuropyridinyl)pyridine-N,C′]; iridium(III)bis[(methyl-d₃)(phenyl)pyridine-N,C′][(isopropyl-d₁)(methyl-d₃)(benzofuropyridinyl)pyridine-N,C′]; iridium(III)bis[(dimethyl-d₆)(phenyl)pyridine-N,C′]{(isopropyl-d₇)[(methyl-d₃)benzofuropyridinyl]pyridine-N,C′}; iridium(III)bis[(dimethyl-d₆)(phenyl)pyridine-N,C′]{(isopropyl)(methyl-d₃)[(methyl-d₃)benzofuropyridinyl]pyridine-N,C′}; iridium(III)bis[(dimethyl-d₆)(phenyl)pyridine-N,C′]{(isopropyl-d₇)(methyl-d₃)[(methyl-d₃)benzofuropyridinyl]pyridine-N,C′}; iridium(III)bis[(dimethyl-d₆)(phenyl)pyridine-N,C′]{(isopropyl-d₇)(methyl-d₃)[(methyl-d₃)benzofuropyridinyl]pyridine-N,C″}; iridium(III)bis[(methyl-d₃)(phenyl)pyridine-N,C′]{(isopropyl-d₇)(methyl-d₃)[(methyl-d₃)benzofuropyridinyl]pyridine-N,C′}; iridium(III)bis(phenylpyridine-N,C′{[(dimethyl-d₆)benzofuropyridinyl]pyridine-N,C′}; iridium(III)bis[(dimethyl-d₆)(phenyl)pyridine-N,C′]{(isopropyl)[(isopropyl)benzofuropyridinyl]pyridine-N,C″}; iridium(III)bis[(dimethyl-d₆)(phenyl)pyridine-N,C′]{(isopropyl-d₁)[(isopropyl-d₁)benzofuropyridinyl]pyridine-N,C′}; iridium(III)bis[(methyl-d₃)(phenyl)pyridine-N,C′]{(isopropyl-d₁)[(isopropyl-d₁)benzofuropyridinyl]pyridine-N,C″}; iridium(III)bis[(phenyl)pyridine-N,C′]{[(dimethyl-d₆)benzofuropyridinyl]pyridine-N,C″}; iridium(III)bis[(phenyl)pyridine-N,C′]{[(dimethyl-d₆)benzofuropyridinyl]pyridine-N,C′}; iridium(III)bis[(methyl-d₃)(phenyl)pyridine-N,C′][diphenylpyridine-N,C′]; iridium(III)bis[phenylpyridine-N,C′][diphenylpyridine-N,C′]; iridium(III)bis{[methyl(phenyl-d₃)](pyridine-d₄)-N,C′]{[(diphenyl-d₉)(pyridine-d₃)]-N,C′}; iridium(III)bis[phenylpyridine-N,C′]{phenyl[(methyl-d₃)phenyl]pyridine-N,C′]; iridium(III)bis[(methyl)(phenyl)pyridine-N,C′][diphenylpyridine-N,C′]; iridium(III)bis[(dimethyl)(phenyl)pyridine-N,C′][diphenylpyridine-N,C′]; iridium(III)bis[(dimethyl)(phenyl)pyridine-N,C′]{phenyl[(methyl-d₃)phenyl]pyridine-N,C′]; iridium(III)bis[(dimethyl-d₆)(phenyl)pyridine-N,C′][diphenylpyridine-N,C′]; iridium(III)bis[(methyl)(phenyl)pyridine-N,C′][diphenylpyridine-N,C′]; iridium(III)bis[(phenyl)pyridine-N,C′][(fluorophenyl)(methylbenzofuropyridinyl)pyridine-N,C′]; iridium(III)bis[(phenyl)pyridine-N,C′][(fluorophenyl)(methylbenzofuropyridinyl)pyridine-N,C′]; iridium(III)bis[(phenyl)pyridine-N,C′]{(fluorophenyl)[(methyl-d₃)benzofuropyridinyl]pyridine-N,C′}; iridium(III)bis[(phenyl)pyridine-N,C′]{(fluorophenyl)[(methyl-d₃)benzofuropyridinyl]pyridine-N,C′}; iridium(III)bis[(phenyl)pyridine-N,C′]{(phenyl)[(methyl-d₃)benzofuropyridinyl]pyridine-N,C′}; iridium(III)bis[(phenyl)pyridine-N,C′][phenylbipyridine-N,C′]; iridium(III)bis[(isopropylphenyl)pyridine-N,C′][(fluorophenyl)(methylbenzofuropyridinyl)pyridine-N,C′]; iridium(III)bis{[(isopropyl-d₇)phenyl]pyridine-N,C′}{(fluorophenyl)[(methyl-d₃)benzofuropyridinyl]pyridine-N,C′}; iridium(III) bis{[(isopropyl-d₇)phenyl]pyridine-N,C′}{(fluorophenyl)[(methyl-d₃)benzofuropyridinyl]pyridine-N,C′}; iridium(III)bis{[(methylphenyltriazinyl)phenyl]pyridine-N,C′}iridium(III)bis[phenylquinoline-N,C′](acetylacetonate); iridium(III)bis[(methyl)(methylphenyl)quinoline-N,C′](acetylacetonate); iridium(III)bis[(methyl)(methylphenyl)quinoline-N,C′](diethylnonanedionate); iridium(III)bis[(methyl)(methylphenyl)quinoline-N,C′](methyldiethylnonanedionate); iridium(III)bis[(dimethylphenyl)quinoline-N,C′](acetylacetonate); iridium(III)bis[(isobutyl)(dimethylphenyl)quinoline-N,C′](acetylacetonate); iridium(III)bis[(isobutyl)(methylphenyl)quinoline-N,C′](dimethylnonanedionate); iridium(III)bis[(isobutyl)(methylphenyl)quinoline-N,C′](dimethylheptanedionate); iridium(III)bis[(isobutyl)(dimethylphenyl)quinoline-N,C′](acetylacetonate); iridium(III)bis[(isobutyl)(dimethylphenyl)quinoline-N,C′](dimethylnonanedionate); iridium(III)bis[(phenyl)quinoline-N,C′](diethylnonanedionate); iridium(III)bis[(Isopentyl)(dimethylphenyl)quinoline-N,C′](diethylnonanedionate); iridium(III)bis[(isobutyl)(dimethylphenyl)quinoline-N,C′](diethylnonanedionate); iridium(III)bis[(isobutyl)(dimethylphenyl)quinoline-N,C′](bis[cyclopentyl]acetylacetonate); iridium(III)bis[(isobutyl)(dimethylphenyl)quinoline-N,C′](bis[cyclohexyl]acetylacetonate); iridium(III)bis[(diisobutyl)(dimethylphenyl)quinoline-N,C′](acetylacetonate); iridium(III)bis[(dimethyl)(dimethylphenyl)quinoline-N,C′](ethylmethyldecanedionate); iridium(III)bis[(dimethyl)(dimethylphenyl)quinoline-N,C′](dimethylnonanedionate); iridium(III)bis[(dimethyl)[(dimethyl-d₆)phenyl]quinoline-N,C′](acetylacetonate); iridium(III)bis[methyl(diphenyl)dibenzoquinoxaline-N,C′](acetylacetonate); iridium(III)bis[(phenyl)quinoline-N,C′](acetylacetonate); iridium(III)bis[(phenyl)isoquinoline-N,C′](acetylacetonate); iridium(III)bis[(cyclobutyl)(dimethylphenyl)quinoline-N,C′](diethylnonanedionate); iridium(III)bis[(cyclopentyl)(dimethylphenyl)quinoline-N,C′](diethylnonanedionate); iridium(III)bis[(cyclobutyl)(dimethylphenyl)quinoline-N,C′](methyldiethylnonanedionate); iridium(III)bis[(cyclopentyl)(dimethylphenyl)quinoline-N,C′](methyldiethylnonanedionate); iridium(III)bis[(cyclohexyl)(methylphenyl)quinoline-N,C′](acetylacetonate); iridium(III)bis[(methylphenyl)(dimethylphenyl)quinoline-N,C′](diethylnonanedionate); iridium(III)bis[(trifluoropropyl)(dimethylphenyl)quinoline-N,C′](diethylnonanedionate); iridium(III)bis[(trifluoropropyl)(dimethylphenyl)quinoline-N,C′](acetylacetonate); iridium(III)bis[(trifluoropropyl)(dimethylphenyl)quinoline-N,C′][bis(cyclopentyl)acetylacetonate]; iridium(III)bis[(trifluoropropyl)(dimethylphenyl)quinoline-N,C′][bis(cyclohexyl)acetylacetonate]; iridium(III)bis[(trifluoropropyl)(dimethylphenyl)quinoline-N,C′](diethylnonanedionate); iridium(III)bis[(trifluoropropyl)(dimethylphenyl)quinoline-N,C′](acetylacetonate); iridium(III)bis[(trifluoropropyl)(dimethylphenyl)quinoline-N,C′][bis(cyclopentyl)acetylacetonate]; iridium(III)bis[(trifluoropropyl)(dimethylphenyl)quinoline-N,C′][bis(cyclohexyl)acetylacetonate]; iridium(III)bis[(trifluorodimethylbutyl)(dimethylphenyl)quinoline-N,C′](diethylnonanedionate); iridium(III)bis[(trifluorodimethylbutyl)(dimethylphenyl)quinoline-N,C′](acetylacetonate); iridium(III)bis[(trifluorodimethylbutyl)(dimethylphenyl)quinoline-N,C′][bis(cyclohexyl)acetylacetonate]; iridium(III)bis[(trifluorodimethylbutyl)(dimethylphenyl)quinoline-N,C′][bis(cyclopentyl)acetylacetonate]; iridium(III)bis[(trifluorodimethylbutyl)(dimethylphenyl)quinoline-N,C′][diethylnonanedionate]; iridium(III)bis[(trifluorodimethylbutyl)(dimethylphenyl)quinoline-N,C′][acetylacetonate]; iridium(III)bis[(trifluorodimethylbutyl)(dimethylphenyl)quinoline-N,C′][bis(cyclopentyl)acetylacetonate]; iridium(III)bis[(trifluorodimethylbutyl)(dimethylphenyl)quinoline-N,C′][bis(cyclohexyl)acetylacetonate]; iridium(III)bis[(trifluoropropyl)(dimethylphenyl)quinoline-N,C′][bis(ethylmethylisopropylnonanedionate]; iridium(III)bis[(trifluorodimethylbutyl)(dimethylphenyl)quinoline-N,C′][dimethyldiisopropylnonanedionate]; iridium(III)bis[(trifluorodimethylbutyl)(dimethylphenyl)quinoline-N,C′][dimethyldiisopropylnonanedionate]; iridium(III)bis[(isobutyl)(dimethylbenzofuropyridinyl)quinoline-N,C′][dimethyldiisopropylnonanedionate]; iridium(III)bis[(isobutyl)(dimethylbenzofuropyridinyl)quinoline-N,C′][diethylnonanedionate]; iridium(III)bis[(trifluorodimethylbutyl)(dimethylbenzofuropyridinyl)quinoline-N,C′][diethylmethylnonanedionate]; iridium(III)bis[(isobutyl)(dimethylphenyl)quinoline-N,C′][dimethyldiisopropylnonanedionate]; iridium(III)bis[(isopropylphenyl)quinoline-N,C′]{[di(isopropyl)triazinyl](methylbenzofuropyridinyl)pyridine-N,C″}; iridium(III)bis[(isopropyl)(dimethylphenyl)isoquinoline-N,C′][diethylnonanedionate]; iridium(III)bis[(isopropyl)(dimethylphenyl)isoquinoline-N,C′][diethylnonanedionate]; iridium(III)bis[di(isopropyl)(dimethylphenyl)isoquinoline-N,C′][acetylacetonate]; iridium(III)tris[dimethyldibenzoquinoline-N,C′]; iridium(III)bis[(tert-butylphenyl)thienoisoquinoline-N,C′][acetylacetonate]; iridium(III)bis[(phenyl) thienopyridine-N,C′][acetylacetonate]; iridium(III)bis[(dimethylfluorenyl)quinoline-N,C′][acetylacetonate]; iridium(III)bis[(methylnaphthalenyl)pyridine-N,C′][acetylacetonate]; iridium(III)bis{[(methyl)(fluoro)phenyl]isoquinoline-N,C′}[acetylacetonate]; iridium(III)bis[(methyl)(fluoro)(dimethylphenyl)quinoline-N,C′][acetylacetonate]

### (Group 22A)

phenyl(terphenylyl)indolocarbazole; (biphenylyl)(terphenylyl)indolocarbazole; (biphenylyl)(terphenylyl)indolocarbazole; bis(terphenylyl)indolocarbazole; bis(terphenylyl)indolocarbazole; bis(terphenylyl)indolocarbazole; bis(terphenylyl)indolocarbazole; bis(terphenylyl)indolocarbazole; (triphenylenyl)(terphenylyl)indolocarbazole; (dimethylfluorenyl)(terphenylyl)indolocarbazole; (spiro[cyclopentanefluorene]yl)(terphenylyl)indolocarbazole; (spiro[cyclohexanefluorene]yl)(terphenylyl)indolocarbazole; (dimethylfluorenyl)(terphenylyl)indolocarbazole; (spirobi[fluorene]yl)(terphenylyl)indolocarbazole; (dibenzofuranyl)(terphenylyl)indolocarbazole; (dibenzofuranyl)(terphenylyl)indolocarbazole; (dibenzofuranyl)(terphenylyl)indolocarbazole; (dibenzothiophenyl)(terphenylyl)indolocarbazole; (dibenzothiophenyl)(terphenylyl)indolocarbazole; (dibenzothiophenyl)(terphenylyl)indolocarbazole; (dibenzoselenophenyl)(terphenylyl)indolocarbazole; (dibenzoselenophenyl)(terphenylyl)indolocarbazole; (dibenzoselenophenyl)(terphenylyl)indolocarbazole; (phenylcarbazolyl)(terphenylyl)indolocarbazole; [(carbazolyl)phenyl](terphenylyl)indolocarbazole; (benzothienopyridinyl)(terphenylyl)indolocarbazole; (benzothienopyrimidinyl)(terphenylyl)indolocarbazole; (benzothienopyrimidinyl)(terphenylyl)indolocarbazole; (phenylbenzothienopyrimidinyl)(terphenylyl)indolocarbazole; (diphenyltriazinyl)(terphenylyl)indolocarbazole; (phenylquinazolinyl)(terphenylyl)indolocarbazole; (dibenzoquinazolinyl)(terphenylyl)indolocarbazole; (dibenzoquinoxalinyl)(terphenylyl)indolocarbazole; [(phenyl-d₅)biphenylyl](biphenylyl) indolocarbazole; [(phenyl-d₅)biphenylyl](phenyl-d₅)indolocarbazole; [(dimethyl-d₆)fluorenyl](terphenylyl)indolocarbazole; [(dibenzofuranyl)phenyl](terphenylyl)indolocarbazole; [(dibenzothiophenyl)phenyl](terphenylyl)indolocarbazole; [(triphenylenyl)phenyl](terphenylyl)indolocarbazole; [(diphenyltriazinyl)phenyl](terphenylyl)indolocarbazole; (phenyl)(terphenylyl)indolocarbazole; (biphenylyl)(terphenylyl)indolocarbazole; (biphenylyl)(terphenylyl)indolocarbazole; bis(terphenylyl)indolocarbazole; bis(terphenylyl)indolocarbazole; (triphenylenyl)(terphenylyl)indolocarbazole; (dimethylfluorenyl)(terphenylyl)indolocarbazole; (spiro[cyclohexanefluorene]yl)(terphenylyl)indolocarbazole; (spiro[cyclopentanefluorene]yl)(terphenylyl)indolocarbazole; (spirobi[fluorene]yl)(terphenylyl)indolocarbazole; (dimethylfluorenyl)(terphenylyl)indolocarbazole; (dibenzofuranyl)(terphenylyl)indolocarbazole; (dibenzofuranyl)(terphenylyl)indolocarbazole; (dibenzofuranyl)(terphenylyl)indolocarbazole; (dibenzothiophenyl)(terphenylyl)indolocarbazole; (dibenzothiophenyl)(terphenylyl)indolocarbazole; (dibenzoselenophenyl)(terphenylyl)indolocarbazole; (dibenzoselenophenyl)(terphenylyl)indolocarbazole; (dibenzoselenophenyl)(terphenylyl)indolocarbazole; (dibenzoselenophenyl)(terphenylyl)indolocarbazole; (phenylcarbazolyl)(terphenylyl)indolocarbazole; [(carbazolyl)phenyl](terphenylyl)indolocarbazole; (benzothienopyridinyl)(terphenylyl)indolocarbazole; (benzothienopyrimidinyl)(terphenylyl)indolocarbazole; (benzothienopyrimidinyl)(terphenylyl)indolocarbazole; (phenylbenzothienopyrimidinyl)(terphenylyl)indolocarbazole; (diphenylbenzothienopyrimidinyl)(terphenylyl)indolocarbazole; (diphenylbenzothienopyrimidinyl)(terphenylyl)indolocarbazole; (diphenyltriazinyl)(terphenylyl)indolocarbazole; (phenylpyridotriazinyl)(terphenylyl)indolocarbazole; (dibenzopyrimidinyl)(terphenylyl)indolocarbazole; (dibenzoquinoxalinyl)(terphenylyl)indolocarbazole; [(dimethyl-d₆) fluorenyl](terphenylyl)indolocarbazole; [(diphenyltriazinyl)phenyl](terphenylyl)indolocarbazole; [(phenyl-d₅)biphenylyl](biphenylyl)indolocarbazole; [(phenyl-d₅)biphenylyl](phenyl-d₅)indolocarbazole; (phenyl)(terphenylyl)indolocarbazole; (biphenylyl)(terphenylyl)indolocarbazole; (biphenylyl)(terphenylyl)indolocarbazole; bis(terphenylyl)indolocarbazole; bis(terphenylyl)indolocarbazole; (triphenylenyl)(terphenylyl)indolocarbazole; (dimethylfluorenyl)(terphenylyl)indolocarbazole; (spirobi[fluorene]yl)(terphenylyl)indolocarbazole; (spiro[cyclopentanefluorene]yl)(terphenylyl)indolocarbazole; (dibenzofuranyl)(terphenylyl)indolocarbazole; (dibenzofuranyl)(terphenylyl)indolocarbazole; (dibenzofuranyl)(terphenylyl)indolocarbazole; (dibenzothiophenyl)(terphenylyl)indolocarbazole; (dibenzothiophenyl)(terphenylyl)indolocarbazole; (dibenzothiophenyl)(terphenylyl)indolocarbazole; (diphenyltriazinyl)(terphenylyl)indolocarbazole; (phenylquinazolinyl)(terphenylyl)indolocarbazole; (benzothienopyrimidinyl)(terphenylyl)indolocarbazole; (phenyl)(terphenylyl)indolocarbazole; (biphenylyl)(terphenylyl)indolocarbazole; (biphenylyl)(terphenylyl)indolocarbazole; bis(terphenylyl)indolocarbazole; bis(terphenylyl)indolocarbazole; (triphenylenyl)(terphenylyl)indolocarbazole; (dimethylfluorenyl)(terphenylyl)indolocarbazole; (spirobi[fluorene]yl)(terphenylyl)indolocarbazole; (spiro[cyclopentanefluorene]yl)(terphenylyl)indolocarbazole; (dibenzofuranyl)(terphenylyl)indolocarbazole; (dibenzofuranyl)(terphenylyl)indolocarbazole; (dibenzofuranyl)(terphenylyl)indolocarbazole; (dibenzothiophenyl)(terphenylyl)indolocarbazole; (dibenzothiophenyl)(terphenylyl)indolocarbazole; (dibenzothiophenyl)(terphenylyl)indolocarbazole; (diphenyltriazinyl)(terphenylyl)indolocarbazole; (phenylquinazolinyl)(terphenylyl)indolocarbazole; (benzothienopyrimidinyl)(terphenylyl)indolocarbazole; (phenyl)(terphenylyl)indolocarbazole; (biphenylyl)(terphenylyl)indolocarbazole; (biphenylyl)(terphenylyl)indolocarbazole; bis(terphenylyl)indolocarbazole; (dimethylfluorenyl)(terphenylyl)indolocarbazole; (dibenzofuranyl)(terphenylyl)indolocarbazole; (dibenzothiophenyl)(terphenylyl)indolocarbazole; (dibenzothiophenyl)(terphenylyl)indolocarbazole; (benzothienopyridinyl)(terphenylyl)indolocarbazole; (benzothienopyrimidinyl)(terphenylyl)indolocarbazole; (diphenyltriazinyl)(terphenylyl)indolocarbazole; (phenylquinazolinyl)(terphenylyl)indolocarbazole; [(phenyl-d₅)biphenylyl](biphenylyl)indolocarbazole; [(cyclohexyl-d₅)biphenylyl](phenyl-d₅)indolocarbazole; (phenyl)(terphenylyl)indolocarbazole; (biphenylyl)(terphenylyl)indolocarbazole; (biphenylyl)(terphenylyl)indolocarbazole; (dibenzothiophenyl)(terphenylyl)indolocarbazole; (dibenzothiophenyl)(terphenylyl)indolocarbazole; (dibenzofuranyl)(diphenyltriazinyl)biphenyl; (dibenzothiophenyl)(diphenyltriazinyl)biphenyl; (dibenzoselenophenyl)(diphenyltriazinyl)biphenyl; (dibenzofuranyl)(diphenyltriazinyl)terbenzene; (dibenzothiophenyl)(diphenyltriazinyl)terbenzene; (dibenzoselenophenyl)(diphenyltriazinyl)terbenzene; (phenyldibenzofuranyl)(diphenyltriazinyl)biphenyl; (phenyldibenzothiophenyl)(diphenyltriazinyl)biphenyl; (phenyldibenzoselenophenyl)(diphenyltriazinyl)biphenyl; [(biphenylyl)dibenzofuranyl](diphenyltriazinyl)biphenyl; [(biphenylyl)dibenzothiophenyl](diphenyltriazinyl)biphenyl; [(biphenylyl)dibenzoselenophenyl](diphenyltriazinyl)biphenyl; [(biphenylyl)dibenzofuranyl](diphenyltriazinyl)biphenyl; [(biphenylyl)dibenzothiophenyl](diphenyltriazinyl)biphenyl; [(biphenylyl)dibenzoselenophenyl](diphenyltriazinyl)biphenyl; (phenyldibenzofuranyl)(diphenyltriazinyl)terphenyl; (phenyldibenzothiophenyl)(diphenyltriazinyl)terphenyl; (phenyldibenzoselenophenyl)(diphenyltriazinyl)terphenyl; (phenyldibenzofuranyl)(diphenyltriazinyl)terphenyl; (phenyldibenzothiophenyl)(diphenyltriazinyl)terphenyl; (phenyldibenzoselenophenyl)(diphenyltriazinyl)terphenyl; [(biphenylyl)dibenzofuranyl](diphenyltriazinyl)terphenyl; [(biphenylyl)dibenzothiophenyl](diphenyltriazinyl)terphenyl; [(biphenylyl)dibenzoselenophenyl](diphenyltriazinyl)terphenyl; [phenyl(biphenylyl)triazinyl](dibenzofuranyl)biphenyl; [phenyl(biphenylyl)triazinyl](dibenzothiophenyl)biphenyl; [phenyl(biphenylyl)triazinyl](dibenzoselenophenyl)biphenyl; [phenyl(biphenylyl)triazinyl](dibenzofuranyl)biphenyl; [phenyl(biphenylyl)triazinyl](dibenzothiophenyl)biphenyl; [phenyl(biphenylyl)triazinyl](dibenzoselenophenyl)biphenyl; [bis(biphenylyl)triazinyl](dibenzofuranyl)biphenyl; [bis(biphenylyl)triazinyl](dibenzothiophenyl)biphenyl; [bis(biphenylyl)triazinyl](dibenzoselenophenyl)biphenyl; [phenyl(dimethylfluorenyl)triazinyl](dibenzofuranyl)biphenyl; [phenyl(dimethylfluorenyl)triazinyl](dibenzothiophenyl)biphenyl; [phenyl(dimethylfluorenyl)triazinyl](dibenzoselenophenyl)biphenyl; [phenyl(biphenylyl)triazinyl](phenyldibenzofuranyl)biphenyl; [phenyl(biphenylyl)triazinyl](phenyldibenzothiophenyl)biphenyl; [phenyl(biphenylyl)triazinyl](phenyldibenzoselenophenyl)biphenyl; [phenyl(biphenylyl)triazinyl](phenyldibenzofuranyl)biphenyl; [phenyl(biphenylyl)triazinyl](phenyldibenzothiophenyl)biphenyl; [phenyl(biphenylyl)triazinyl](phenyldibenzoselenophenyl)biphenyl; [phenyl(dimethylfluorenyl)triazinyl](phenyldibenzofuranyl)biphenyl; [phenyl(dimethylfluorenyl)triazinyl](phenyldibenzothiophenyl)biphenyl; [phenyl(dimethylfluorenyl)triazinyl](phenyldibenzoselenophenyl)biphenyl; [phenyl(biphenylyl)triazinyl](dibenzofuranyl)terphenyl; [phenyl(biphenylyl)triazinyl](dibenzothiophenyl)terphenyl; [phenyl(biphenylyl)triazinyl](dibenzoselenophenyl)terphenyl; [phenyl(biphenylyl)triazinyl](dibenzofuranyl)terphenyl; [phenyl(biphenylyl)triazinyl](dibenzothiophenyl)terphenyl; [phenyl(biphenylyl)triazinyl](dibenzoselenophenyl)terphenyl; [phenyl(dimethylfluorenyl)triazinyl](dibenzofuranyl)terphenyl; [phenyl(dimethylfluorenyl)triazinyl](dibenzothiophenyl)terphenyl; [phenyl(dimethylfluorenyl)triazinyl](dibenzoselenophenyl)terphenyl; [phenyl(biphenylyl)triazinyl][(biphenylyl)dibenzofuranyl)]biphenyl; [phenyl(biphenylyl)triazinyl][(biphenylyl)dibenzothiophenyl)]biphenyl; [phenyl(biphenylyl)triazinyl][(biphenylyl)dibenzoselenophenyl)]biphenyl; [phenyl(biphenylyl)triazinyl][(biphenylyl)dibenzofuranyl)]biphenyl; [phenyl(biphenylyl)triazinyl][(biphenylyl)dibenzothiophenyl)]biphenyl; [phenyl(biphenylyl)triazinyl][(biphenylyl)dibenzoselenophenyl)]biphenyl; [phenyl(dimethylfluorenyl)triazinyl][(biphenylyl)dibenzofuranyl)]biphenyl; [phenyl(dimethylfluorenyl)triazinyl][(biphenylyl)dibenzothiophenyl]biphenyl; [phenyl(dimethylfluorenyl)triazinyl][(biphenylyl)dibenzoselenophenyl]biphenyl; [phenyl(biphenylyl)triazinyl](dibenzofuranyl)terphenyl; [phenyl(biphenylyl)triazinyl](dibenzothiophenyl)terphenyl; [phenyl(biphenylyl)triazinyl](dibenzoselenophenyl)terphenyl; (dibenzofuranyl)[phenyl(biphenylyl)triazinyl]terphenyl; (dibenzothiophenyl)[phenyl(biphenylyl)triazinyl]terphenyl; (dibenzoselenophenyl)[phenyl(biphenylyl)triazinyl]terphenyl; (diphenyltriazinyl)(dibenzofuranyl)biphenyl; (diphenyltriazinyl)(dibenzothiophenyl)biphenyl; (diphenyltriazinyl)(dibenzoselenophenyl)biphenyl; (diphenyltriazinyl)(dibenzofuranyl)biphenyl; (diphenyltriazinyl)(dibenzothiophenyl)biphenyl; (diphenyltriazinyl)(dibenzoselenophenyl)biphenyl; (diphenyltriazinyl)(dibenzofuranyl)biphenyl; (diphenyltriazinyl)(dibenzothiophenyl)biphenyl; (diphenyltriazinyl)(dibenzoselenophenyl)biphenyl; (diphenyltriazinyl)(dibenzofuranyl)terphenyl; (diphenyltriazinyl)(dibenzothiophenyl)terphenyl; (diphenyltriazinyl)(dibenzoselenophenyl)terphenyl; (diphenyltriazinyl)(dibenzofuranyl)terphenyl; (diphenyltriazinyl)(dibenzothiophenyl)terphenyl; (diphenyltriazinyl)(dibenzoselenophenyl)terphenyl; (diphenyltriazinyl)(dibenzofuranyl)terphenyl; (diphenyltriazinyl)(dibenzothiophenyl)terphenyl; (diphenyltriazinyl)(dibenzoselenophenyl)terphenyl; (diphenyltriazinyl)(diphenyldibenzofuranyl)biphenyl; (diphenyltriazinyl)(diphenyldibenzothiophenyl)biphenyl; (diphenyltriazinyl)(diphenyldibenzoselenophenyl)biphenyl; (diphenyltriazinyl)(diphenyldibenzofuranyl)biphenyl; (diphenyltriazinyl)(diphenyldibenzothiophenyl)biphenyl; (diphenyltriazinyl)(diphenyldibenzoselenophenyl)biphenyl; (diphenyltriazinyl)(phenyldibenzofuranyl)biphenyl; (diphenyltriazinyl)(phenyldibenzothiophenyl)biphenyl; (diphenyltriazinyl)(phenyldibenzoselenophenyl)biphenyl; (diphenyltriazinyl)(phenyldibenzofuranyl)(pyridinyl)phenyl; (diphenyltriazinyl)(phenyldibenzothiophenyl)(pyridinyl)phenyl; (diphenyltriazinyl)(phenyldibenzoselenophenyl)(pyridinyl)phenyl; (diphenyltriazinyl)(phenyldibenzofuranyl)(pyridinyl)phenyl; (diphenyltriazinyl)(phenyldibenzothiophenyl)(pyridinyl)phenyl; (diphenyltriazinyl)(phenyldibenzoselenophenyl)(pyridinyl)phenyl; (diphenyltriazinyl)[(pyridinyl)dibenzofuranyl]biphenyl; (diphenyltriazinyl)[(pyridinyl)dibenzothiophenyl]biphenyl; (diphenyltriazinyl)[(pyridinyl)dibenzoselenophenyl]biphenyl; (diphenyltriazinyl)(dibenzofuranyl)terphenyl; (diphenyltriazinyl)(dibenzothiophenyl)terphenyl; (diphenyltriazinyl)(dibenzoselenophenyl)terphenyl; (dibenzofuranyl)(diphenyltriazinyl)terphenyl; (dibenzothiophenyl)(diphenyltriazinyl)terphenyl; (dibenzoselenophenyl)(diphenyltriazinyl)terphenyl; (diphenyltriazinyl)(dibenzofuranyl)(dimethylfluorenyl)benzene; (diphenyltriazinyl)(dibenzothiophenyl)(dimethylfluorenyl)benzene; (diphenyltriazinyl)(dibenzoselenophenyl)(dimethylfluorenyl)benzene; (diphenyltriazinyl)(dimethylfluorenyl)biphenyl; (diphenyltriazinyl)(spiro[cyclopentanefluorene]yl)biphenyl; (diphenyltriazinyl)(dimethylfluorenyl)terphenyl; (diphenyltriazinyl)(diphenylfluorenyl)biphenyl; [phenyl(biphenylyl)triazinyl](dimethylfluorenyl)biphenyl; [phenyl(dimethylfluorenyl)triazinyl](dimethylfluorenyl)terphenyl; (diphenyltriazinyl)[bis(diphenylfluorenyl)]benzene; (diphenyltriazinyl)(diphenylfluorenyl)terbenzene; (diphenyltriazinyl)(triphenylenyl)biphenyl; phenyl[(diphenyltriazinyl)phenyl]indolocarbazole; (biphenylyl)[(diphenyltriazinyl)phenyl]indolocarbazole; (biphenylyl)[(diphenyltriazinyl)phenyl]indolocarbazole; (dimethylfluorenyl)[(diphenyltriazinyl)phenyl]indolocarbazole; (dibenzothiophenyl)[(diphenyltriazinyl)phenyl]indolocarbazole; (dibenzofuranyl)[(diphenyltriazinyl)phenyl]indolocarbazole; (dibenzothiophenyl)[(diphenyltriazinyl)phenyl]indolocarbazole; (phenyl)[(diphenyltriazinyl)biphenyl]indolocarbazole; (phenyl)[(diphenyltriazinyl)terphenyl]indolocarbazole; (phenyl)[(diphenyltriazinyl)terphenyl]indolocarbazole; (phenyl)[((phenyl)(biphenylyl)triazinyl)phenyl]indolocarbazole; (phenyl)[(di(biphenylyl)triazinyl)phenyl]indolocarbazole; (phenyl)[((phenyl)(biphenylyl)triazinyl)phenyl]indolocarbazole; (phenyl)[((phenyl)(naphthyl)triazinyl)phenyl]indolocarbazole; (phenylcarbazolyl)[(diphenyltriazinyl)phenyl]indolocarbazole; (phenylcarbazolyl)[(diphenyltriazinyl)phenyl]indolocarbazole; (phenylcarbazolyl)[(diphenyltriazinyl)phenyl]indolocarbazole; di(phenyl)(triphenylenylbiphenylyl)triazine; (phenyl)(biphenylyl)(triphenylenylphenyl)triazine; (phenyl)(biphenylyl)(triphenylenylphenyl)triazine; di(biphenylyl)(triphenylenylphenyl)triazine; di(phenyl)(triphenylenylbiphenylyl)triazine; di(biphenylyl)(triphenylenyl)triazine; di(biphenylyl)(triphenylenyl)triazine; (phenyl)(biphenylyl)(triphenylenyl)triazine; (phenyl)(biphenylyl)(triphenylenyl)triazine; (naphthyl)[(diphenyltriazinyl)phenyl]indolocarbazole; (phenyl)(diphenyltriazinyl)dibenzofuran; (phenyl)(diphenyltriazinyl)dibenzothiophene; (phenyl)(diphenyltriazinyl)dibenzoselenophene; (phenyl)[(phenyl)(biphenylyl)triazinyl]dibenzofuran; (phenyl)[(phenyl)(biphenylyl)triazinyl]dibenzothiophene; (phenyl)[(phenyl)(biphenylyl)triazinyl]dibenzoselenophene; (phenyl)[(phenyl)(biphenylyl)triazinyl]dibenzofuran; (phenyl)[(phenyl)(biphenylyl)triazinyl]dibenzothiophene; (phenyl)[(phenyl)(biphenylyl)triazinyl]dibenzoselenophene; (phenyl)[di(biphenylyl)triazinyl]dibenzofuran; (phenyl)[di(biphenylyl)triazinyl]dibenzothiophene; (phenyl)[di(biphenylyl)triazinyl]dibenzoselenophene; (phenyl)[di(biphenylyl)triazinyl]dibenzofuran; (phenyl)[di(biphenylyl)triazinyl]dibenzothiophene; (phenyl)[di(biphenylyl)triazinyl]dibenzoselenophene; (phenyl)[di(biphenylyl)triazinyl]dibenzofuran; (phenyl)[di(biphenylyl)triazinyl]dibenzothiophene; (phenyl)[di(biphenylyl)triazinyl]dibenzoselenophene; (phenyl)[(diphenyltriazinyl)phenyl]dibenzofuran; (phenyl)[(diphenyltriazinyl)phenyl]dibenzothiophene; (phenyl)[(diphenyltriazinyl)phenyl]dibenzoselenophene; (phenyl)[(phenyl)(biphenylyl)triazinylphenyl]dibenzofuran; (phenyl)[(phenyl)(biphenylyl)triazinylphenyl]dibenzothiophene; (phenyl)[(phenyl)(biphenylyl)triazinylphenyl]dibenzoselenophene; (phenyl)[(phenyl)(biphenylyl)triazinylphenyl]dibenzofuran; (phenyl)[(phenyl)(biphenylyl)triazinylphenyl]dibenzothiophene; (phenyl)[(phenyl)(biphenylyl)triazinylphenyl]dibenzoselenophene; (phenyl)[(diphenyltriazinyl)phenyl]dibenzofuran; (phenyl)[(diphenyltriazinyl)phenyl]dibenzothiophene; (phenyl)[(diphenyltriazinyl)phenyl]dibenzoselenophene; (phenyl)[(phenyl)(biphenylyl)triazinylphenyl]dibenzofuran; (phenyl)[(phenyl)(biphenylyl)triazinylphenyl]dibenzothiophene; (phenyl)[(phenyl)(biphenylyl)triazinylphenyl]dibenzoselenophene; (phenyl)[(phenyl)(biphenylyl)triazinylphenyl]dibenzofuran; (phenyl)[(phenyl)(biphenylyl)triazinylphenyl]dibenzothiophene; (phenyl)[(phenyl)(biphenylyl)triazinylphenyl]dibenzoselenophene; (phenyl)[(diphenyltriazinyl)biphenyl]dibenzofuran; (phenyl)[(diphenyltriazinyl)biphenyl]dibenzothiophene; (phenyl)[(diphenyltriazinyl)biphenyl]dibenzoselenophene; (phenyl)[(phenyl)(biphenylyl)triazinylbiphenyl]dibenzofuran; (phenyl)[(phenyl)(biphenylyl)triazinylbiphenyl]dibenzothiophene; (phenyl)[(phenyl)(biphenylyl)triazinylbiphenyl]dibenzoselenophene; (phenyl)[(phenyl)(biphenylyl)triazinylbiphenyl]dibenzofuran; (phenyl)[(phenyl)(biphenylyl)triazinylbiphenyl]dibenzothiophene; (phenyl)[(phenyl)(biphenylyl)triazinylbiphenyl]dibenzoselenophene; (phenyl)[(diphenyltriazinyl)biphenyl]dibenzofuran; (phenyl)[(diphenyltriazinyl)biphenyl]dibenzothiophene; (phenyl)[(diphenyltriazinyl)biphenyl]dibenzoselenophene; (phenyl)[(phenyl)(biphenylyl)triazinylbiphenyl]dibenzofuran; (phenyl)[(phenyl)(biphenylyl)triazinylbiphenyl]dibenzothiophene; (phenyl)[(phenyl)(biphenylyl)triazinylbiphenyl]dibenzoselenophene; (phenyl)[(phenyl)(biphenylyl)triazinylbiphenyl]dibenzofuran; (phenyl)[(phenyl)(biphenylyl)triazinylbiphenyl]dibenzothiophene; (phenyl)[(phenyl)(biphenylyl)triazinylbiphenyl]dibenzoselenophene; (phenyl)[(diphenyltriazinyl)biphenyl]dibenzofuran; (phenyl)[(diphenyltriazinyl)biphenyl]dibenzothiophene; (phenyl)[(diphenyltriazinyl)biphenyl]dibenzoselenophene; (phenyl)[(phenyl)(biphenylyl)triazinylbiphenyl]dibenzofuran; (phenyl)[(phenyl)(biphenylyl)triazinylbiphenyl]dibenzothiophene; (phenyl)[(phenyl)(biphenylyl)triazinylbiphenyl]dibenzoselenophene; (phenyl)[(phenyl)(biphenylyl)triazinylbiphenyl]dibenzofuran; (phenyl)[(phenyl)(biphenylyl)triazinylbiphenyl]dibenzothiophene; (phenyl)[(phenyl)(biphenylyl)triazinylbiphenyl]dibenzoselenophene; (biphenylyl)(diphenyltriazinyl)dibenzofuran; (biphenylyl)(diphenyltriazinyl)dibenzothiophene; (biphenylyl)(diphenyltriazinyl)dibenzoselenophene; (biphenylyl)[(phenyl)(biphenylyl)triazinyl]dibenzofuran; (biphenylyl)[(phenyl)(biphenylyl)triazinyl]dibenzothiophene; (biphenylyl)[(phenyl)(biphenylyl)triazinyl]dibenzoselenophene; (biphenylyl)[(phenyl)(biphenylyl)triazinyl]dibenzofuran; (biphenylyl)[(phenyl)(biphenylyl)triazinyl]dibenzothiophene; (biphenylyl)[(phenyl)(biphenylyl)triazinyl]dibenzoselenophene; (phenyl)[(diphenyltriazinyl)phenyl]dibenzofuran; (phenyl)[(diphenyltriazinyl)phenyl]dibenzothiophene; (phenyl)[(diphenyltriazinyl)phenyl]dibenzoselenophene; (biphenylyl)[(phenyl)(biphenylyl)triazinylphenyl]dibenzofuran; (biphenylyl)[(phenyl)(biphenylyl)triazinylphenyl]dibenzothiophene; (biphenylyl)[(phenyl)(biphenylyl)triazinylphenyl]dibenzoselenophene; (biphenylyl)[(phenyl)(biphenylyl)triazinylphenyl]dibenzofuran; (biphenylyl)[(phenyl)(biphenylyl)triazinylphenyl]dibenzothiophene; (biphenylyl)[(phenyl)(biphenylyl)triazinylphenyl]dibenzoselenophene; (biphenylyl)[di(phenyl)triazinylphenyl]dibenzofuran; (biphenylyl)[di(phenyl)triazinylphenyl]dibenzothiophene; (biphenylyl)[di(phenyl)triazinylphenyl]dibenzoselenophene; (biphenylyl)[(phenyl)(biphenylyl)triazinylphenyl]dibenzofuran; (biphenylyl)[(phenyl)(biphenylyl)triazinylphenyl]dibenzothiophene; (biphenylyl)[(phenyl)(biphenylyl)triazinylphenyl]dibenzoselenophene; (biphenylyl)[(phenyl)(biphenylyl)triazinylphenyl]dibenzofuran; (biphenylyl)[(phenyl)(biphenylyl)triazinylphenyl]dibenzothiophene; (biphenylyl)[(phenyl)(biphenylyl)triazinylphenyl]dibenzoselenophene; (biphenylyl)[di(phenyl)triazinyl]dibenzofuran; (biphenylyl)[di(phenyl)triazinyl]dibenzothiophene; (biphenylyl)[di(phenyl)triazinyl]dibenzoselenophene; (biphenylyl)[(phenyl)(biphenylyl)triazinyl]dibenzofuran; (biphenylyl)[(phenyl)(biphenylyl)triazinyl]dibenzothiophene; (biphenylyl)[(phenyl)(biphenylyl)triazinyl]dibenzoselenophene; (biphenylyl)[(phenyl)(biphenylyl)triazinyl]dibenzofuran; (biphenylyl)[(phenyl)(biphenylyl)triazinyl]dibenzothiophene; (biphenylyl)[(phenyl)(biphenylyl)triazinyl]dibenzoselenophene; (biphenylyl)[di(phenyl)triazinylphenyl]dibenzofuran; (biphenylyl)[di(phenyl)triazinylphenyl]dibenzothiophene; (biphenylyl)[di(phenyl)triazinylphenyl]dibenzoselenophene; (biphenylyl)[(phenyl)(biphenylyl)triazinylphenyl]dibenzofuran; (biphenylyl)[(phenyl)(biphenylyl)triazinylphenyl]dibenzothiophene; (biphenylyl)[(phenyl)(biphenylyl)triazinylphenyl]dibenzoselenophene; (biphenylyl)[(phenyl)(biphenylyl)triazinylphenyl]dibenzofuran; (biphenylyl)[(phenyl)(biphenylyl)triazinylphenyl]dibenzothiophene; (biphenylyl)[(phenyl)(biphenylyl)triazinylphenyl]dibenzoselenophene; (biphenylyl)[di(phenyl)triazinylphenyl]dibenzofuran; (biphenylyl)[di(phenyl)triazinylphenyl]dibenzothiophene; (biphenylyl)[di(phenyl)triazinylphenyl]dibenzoselenophene; (biphenylyl)[(phenyl)(biphenylyl)triazinylphenyl]dibenzofuran; (biphenylyl)[(phenyl)(biphenylyl)triazinylphenyl]dibenzothiophene; (biphenylyl)[(phenyl)(biphenylyl)triazinylphenyl]dibenzoselenophene; (biphenylyl)[(phenyl)(biphenylyl)triazinylphenyl]dibenzofuran; (biphenylyl)[(phenyl)(biphenylyl)triazinylphenyl]dibenzothiophene; (biphenylyl)[(phenyl)(biphenylyl)triazinylphenyl]dibenzoselenophene; (phenyl)[(phenyl)(dimethylfluorenyl)triazinyl]dibenzofuran; (phenyl)[(phenyl)(dimethylfluorenyl)triazinyl]dibenzothiophene; (phenyl)[(phenyl)(dimethylfluorenyl)triazinyl]dibenzoselenophene; (phenyl)[(biphenylyl)(dimethylfluorenyl)triazinyl]dibenzofuran; (phenyl)[(biphenylyl)(dimethylfluorenyl)triazinyl]dibenzothiophene; (phenyl)[(biphenylyl)(dimethylfluorenyl)triazinyl]dibenzoselenophene; (phenyl)[(biphenylyl)(dimethylfluorenyl)triazinyl]dibenzofuran; (phenyl)[(biphenylyl)(dimethylfluorenyl)triazinyl]dibenzothiophene; (phenyl)[(biphenylyl)(dimethylfluorenyl)triazinyl]dibenzoselenophene; (phenyl)[(phenyl)(dimethylfluorenyl)triazinylphenyl]dibenzofuran; (phenyl)[(phenyl)(dimethylfluorenyl)triazinylphenyl]dibenzothiophene; (phenyl)[(phenyl)(dimethylfluorenyl)triazinylphenyl]dibenzoselenophene; (phenyl)[bis(dimethylfluorenyl)triazinylphenyl]dibenzofuran; (phenyl)[bis(dimethylfluorenyl)triazinylphenyl]dibenzothiophene; (phenyl)[bis(dimethylfluorenyl)triazinylphenyl]dibenzoselenophene; (biphenylyl)[(phenyl)(dimethylfluorenyl)triazinyl]dibenzofuran; (biphenylyl)[(phenyl)(dimethylfluorenyl)triazinyl]dibenzothiophene; (biphenylyl)[(phenyl)(dimethylfluorenyl)triazinyl]dibenzoselenophene; (biphenylyl)[(phenyl)(dimethylfluorenyl)triazinyl]dibenzofuran; (biphenylyl)[(phenyl)(dimethylfluorenyl)triazinyl]dibenzothiophene; (biphenylyl)[(phenyl)(dimethylfluorenyl)triazinyl]dibenzoselenophene; (biphenylyl)[(phenyl)(dimethylfluorenyl)triazinylphenyl]dibenzofuran; (biphenylyl)[(phenyl)(dimethylfluorenyl)triazinylphenyl]dibenzothiophene; (biphenylyl)[(phenyl)(dimethylfluorenyl)triazinylphenyl]dibenzoselenophene; (biphenylyl)[(phenyl)(dimethylfluorenyl)triazinylphenyl]dibenzofuran; (biphenylyl)[(phenyl)(dimethylfluorenyl)triazinylphenyl]dibenzothiophene; (biphenylyl)[(phenyl)(dimethylfluorenyl)triazinylphenyl]dibenzoselenophene; (phenyl)[(phenyl)(dimethylfluorenylphenyl)triazinyl]dibenzofuran; (phenyl)[(phenyl)(dimethylfluorenylphenyl)triazinyl]dibenzothiophene; (phenyl)[(phenyl)(dimethylfluorenylphenyl)triazinyl]dibenzoselenophene; (phenyl)[(phenyl)(dimethylfluorenyl)triazinyl]dibenzofuran; (phenyl)[(phenyl)(dimethylfluorenyl)triazinyl]dibenzothiophene; (phenyl)[(phenyl)(dimethylfluorenyl)triazinyl]dibenzoselenophene; (biphenylyl)[(phenyl)(dimethylfluorenyl)triazinyl]dibenzofuran; (biphenylyl)[(phenyl)(dimethylfluorenyl)triazinyl]dibenzothiophene; (biphenylyl)[(phenyl)(dimethylfluorenyl)triazinyl]dibenzoselenophene; (biphenylyl)[(phenyl)(dimethylfluorenyl)triazinylphenyl]dibenzofuran; (biphenylyl)[(phenyl)(dimethylfluorenyl)triazinylphenyl]dibenzothiophene; (biphenylyl)[(phenyl)(dimethylfluorenyl)triazinylphenyl]dibenzoselenophene; (phenyl)[(dimethylfluorenyl)terphenyl]dibenzofuran; (phenyl)[(dimethylfluorenyl)terphenyl]dibenzothiophene; (phenyl)[(dimethylfluorenyl)terphenyl]dibenzoselenophene; (phenyl)[(phenyl)(dimethylfluorenyl)triazinyl]dibenzofuran; (phenyl)[(phenyl)(dimethylfluorenyl)triazinyl]dibenzothiophene; (phenyl)[(phenyl)(dimethylfluorenyl)triazinyl]dibenzoselenophene; (phenyl)[di(phenyl)triazinyl]dibenzofuran; (phenyl)[di(phenyl)triazinyl]dibenzothiophene; (phenyl)[di(phenyl)triazinyl]dibenzoselenophene; (phenyl)[di(phenyl)triazinylphenyl]dibenzofuran; (phenyl)[di(phenyl)triazinylphenyl]dibenzothiophene; (phenyl)[di(phenyl)triazinylphenyl]dibenzoselenophene; (biphenylyl)[di(phenyl)triazinylphenyl]dibenzofuran; (biphenylyl)[di(phenyl)triazinylphenyl]dibenzothiophene; (biphenylyl)[di(phenyl)triazinylphenyl]dibenzoselenophene; di(phenyl)[di(phenyl)triazinylphenyl]dibenzofuran; di(phenyl)[di(phenyl)triazinylphenyl]dibenzothiophene; di(phenyl)[di(phenyl)triazinylphenyl]dibenzoselenophene; (phenyl)[di(phenyl)triazinyl]dibenzofuran; (phenyl)[di(phenyl)triazinyl]dibenzothiophene; (phenyl)[di(phenyl)triazinyl]dibenzoselenophene; (biphenylyl)[di(phenyl)triazinyl]dibenzofuran; (biphenylyl)[di(phenyl)triazinyl]dibenzothiophene; (biphenylyl)[di(phenyl)triazinyl]dibenzoselenophene; (biphenylyl)[di(phenyl)triazinyl]dibenzofuran; (biphenylyl)[di(phenyl)triazinyl]dibenzothiophene; (biphenylyl)[di(phenyl)triazinyl]dibenzoselenophene; di(phenyl)[di(phenyl)triazinylphenyl]dibenzofuran; di(phenyl)[di(phenyl)triazinylphenyl]dibenzothiophene; di(phenyl)[di(phenyl)triazinylphenyl]dibenzoselenophene; (biphenylyl)[di(phenyl)triazinylphenyl]dibenzofuran; (biphenylyl)[di(phenyl)triazinylphenyl]dibenzothiophene; (biphenylyl)[di(phenyl)triazinylphenyl]dibenzoselenophene; (phenyl)[di(phenyl)triazinyl]dibenzofuran; (phenyl)[di(phenyl)triazinyl]dibenzothiophene; (phenyl)[di(phenyl)triazinyl]dibenzoselenophene; (phenyl)[di(phenyl)triazinylphenyl]dibenzofuran; (phenyl)[di(phenyl)triazinylphenyl]dibenzothiophene; (phenyl)[di(phenyl)triazinylphenyl]dibenzoselenophene; (phenyl)[di(phenyl)triazinylphenyl]dibenzofuran; (phenyl)[di(phenyl)triazinylphenyl]dibenzothiophene; (phenyl)[di(phenyl)triazinylphenyl]dibenzoselenophene; (biphenylyl)[di(phenyl)triazinylphenyl]dibenzofuran; (biphenylyl)[di(phenyl)triazinylphenyl]dibenzothiophene; (biphenylyl)[di(phenyl)triazinylphenyl]dibenzoselenophene; (biphenylyl)[di(phenyl)triazinylphenyl]dibenzofuran; (biphenylyl)[di(phenyl)triazinylphenyl]dibenzothiophene; (biphenylyl)[di(phenyl)triazinylphenyl]dibenzoselenophene; (phenyl)[di(phenyl)triazinylphenyl]dibenzofuran; (phenyl)[di(phenyl)triazinylphenyl]dibenzothiophene; (phenyl)[di(phenyl)triazinylphenyl]dibenzoselenophene; (phenyl)[di(phenyl)triazinylpyridyl]dibenzofuran; (phenyl)[di(phenyl)triazinylpyridyl]dibenzothiophene; (phenyl)[di(phenyl)triazinylpyridyl]dibenzoselenophene; (phenyl)[(phenyl)(phenylpyridyl)triazinylpyridyl]dibenzofuran; (phenyl)[(phenyl)(phenylpyridyl)triazinylpyridyl]dibenzothiophene; (phenyl)[(phenyl)(phenylpyridyl)triazinylpyridyl]dibenzoselenophene; (phenyl)[(phenyl)(phenylpyridyl)triazinylpyridyl]dibenzofuran; (phenyl)[(phenyl)(phenylpyridyl)triazinylpyridyl]dibenzothiophene; (phenyl)[(phenyl)(phenylpyridyl)triazinylpyridyl]dibenzoselenophene; (biphenylyl)[(phenyl)(pyridyl)triazinylpyridyl]dibenzofuran; (biphenylyl)[(phenyl)(pyridyl)triazinylpyridyl]dibenzothiophene; (biphenylyl)[(phenyl)(pyridyl)triazinylpyridyl]dibenzoselenophene; (phenyl)[(phenyl)(dimethylindenopyridyl)triazinyl]dibenzofuran; (phenyl)[(phenyl)(dimethylindenopyridyl)triazinyl]dibenzothiophene; (phenyl)[(phenyl)(dimethylindenopyridyl)triazinyl]dibenzoselenophene; (phenyl)[(biphenylyl)(dimethylindenopyridyl)triazinyl]dibenzofuran; (phenyl)[(biphenylyl)(dimethylindenopyridyl)triazinyl]dibenzothiophene; (phenyl)[(biphenylyl)(dimethylindenopyridyl)triazinyl]dibenzoselenophene; (phenyl)[(phenyl)(dimethylindenopyridyl)triazinyl]dibenzofuran; (phenyl)[(phenyl)(dimethylindenopyridyl)triazinyl]dibenzothiophene; (phenyl)[(phenyl)(dimethylindenopyridyl)triazinyl]dibenzoselenophene; (phenyl)[(phenyl)(spirocyclohexanefluorenyl)triazinyl]dibenzofuran; (phenyl)[(phenyl)(spirocyclohexanefluorenyl)triazinyl]dibenzothiophene; (phenyl)[(phenyl)(spirocyclohexanefluorenyl)triazinyl]dibenzoselenophene; (phenyl)[(phenyl)(spirobifluorenyl)triazinyl]dibenzofuran; (phenyl)[(phenyl)(spirobifluorenyl)triazinyl]dibenzothiophene; (phenyl)[(phenyl)(spirobifluorenyl)triazinyl]dibenzoselenophene; (phenyl-d₅)[(diphenyl-d₁₀)triazinylphenyl-do]dibenzofuran; (phenyl-d₅)[(diphenyl-d₁₀)triazinylphenyl-do]dibenzothiophene; (phenyl-d₆)[(diphenyl-d₁₀)triazinylphenyl-d₉]dibenzoselenophene; (biphenyl-d₉)[(diphenyl-d₁₀)triazinylphenyl-d₉]dibenzofuran; (biphenyl-d₉)[(diphenyl-d₁₀)triazinylphenyl-do]dibenzothiophene; (biphenyl-d₉)[(diphenyl-d₁₀)triazinylphenyl-do]dibenzoselenophene; (phenyl)[(phenyl)(dimethyl-d₆)fluorenyl)triazinyl]dibenzofuran; (phenyl)[(phenyl)(dimethyl-d₆)fluorenyl)triazinyl]dibenzothiophene; (phenyl)[(phenyl)(dimethyl-d₆)fluorenyl)triazinyl]dibenzoselenophene; (biphenyl-d₉)[(diphenyl-d₁₀)triazinylphenyl-d₉]dibenzofuran; (biphenyl-d₉)[(diphenyl-d₁₀)triazinylphenyl-do]dibenzothiophene; (biphenyl-d₉)[(diphenyl-d₁₀)triazinylphenyl-do]dibenzoselenophene; (biphenylyl)[(diphenyl-d₁₀)triazinylphenyl]dibenzofuran; (biphenylyl)[(diphenyl-d₁₀)triazinylphenyl]dibenzothiophene; (biphenylyl)[(diphenyl-d₁₀)triazinylphenyl]dibenzoselenophene; (biphenylyl)[di(phenyl)triazinylbiphenyl]dibenzofuran; (biphenylyl)[di(phenyl)triazinylbiphenyl]dibenzothiophene; (biphenylyl)[di(phenyl)triazinylbiphenyl]dibenzoselenophene; (biphenylyl)[bis(fluorophenyl)triazinylphenyl]dibenzofuran; (biphenylyl)[bis(fluorophenyl)triazinylphenyl]dibenzothiophene; (biphenylyl)[bis(fluorophenyl)triazinylphenyl]dibenzoselenophene; (dimethylfluorenyl)[di(phenyl)triazinylphenyl]dibenzofuran; (dimethylfluorenyl)[di(phenyl)triazinylphenyl]dibenzothiophene; (dimethylfluorenyl)[di(phenyl)triazinylphenyl]dibenzoselenophene; (dimethylfluorenyl)[di(phenyl)triazinylbiphenyl]dibenzofuran; (dimethylfluorenyl)[di(phenyl)triazinylbiphenyl]dibenzothiophene; (dimethylfluorenyl)[di(phenyl)triazinylbiphenyl]dibenzoselenophene; (dimethylfluorenyl)[di(phenyl)triazinylbiphenyl]dibenzofuran; (dimethylfluorenyl)[di(phenyl)triazinylbiphenyl]dibenzothiophene; (dimethylfluorenyl)[di(phenyl)triazinylbiphenyl]dibenzoselenophene; (dimethylfluorenyl)[di(phenyl)triazinylbiphenyl]dibenzofuran; (dimethylfluorenyl)[di(phenyl)triazinylbiphenyl]dibenzothiophene; (dimethylfluorenyl)[di(phenyl)triazinylbiphenyl]dibenzoselenophene; (dimethylfluorenyl)[di(phenyl)triazinylpyridyl]dibenzofuran; (dimethylfluorenyl)[di(phenyl)triazinylpyridyl]dibenzothiophene; (dimethylfluorenyl)[di(phenyl)triazinylpyridyl]dibenzoselenophene; (dimethylfluorenyl)[di(phenyl)triazinylphenyl]dibenzofuran; (dimethylfluorenyl)[di(phenyl)triazinylphenyl]dibenzothiophene; (dimethylfluorenyl)[di(phenyl)triazinylphenyl]dibenzoselenophene; (dimethylfluorenyl)[di(phenyl)triazinylphenyl]dibenzofuran; (dimethylfluorenyl)[di(phenyl)triazinylphenyl]dibenzothiophene; (dimethylfluorenyl)[di(phenyl)triazinylphenyl]dibenzoselenophene; (dimethylfluorenyl)[di(phenyl)triazinylphenyl]dibenzofuran; (dimethylfluorenyl)[di(phenyl)triazinylphenyl]dibenzothiophene; (dimethylfluorenyl)[di(phenyl)triazinylphenyl]dibenzoselenophene; (dimethylfluorenyl)[di(phenyl)triazinylbiphenyl]dibenzofuran; (dimethylfluorenyl)[di(phenyl)triazinylbiphenyl]dibenzothiophene; (dimethylfluorenyl)[di(phenyl)triazinylbiphenyl]dibenzoselenophene; (dimethylfluorenyl)[(phenyl)(biphenylyl)triazinylphenyl]dibenzofuran; (dimethylfluorenyl)[(phenyl)(biphenylyl)triazinylphenyl]dibenzothiophene; (dimethylfluorenyl)[(phenyl)(biphenylyl)triazinylphenyl]dibenzoselenophene; (dimethylfluorenyl)[(phenyl)(dimethylfluorenyl)triazinylphenyl]dibenzofuran; (dimethylfluorenyl)[(phenyl)(dimethylfluorenyl)triazinylphenyl]dibenzothiophene; (dimethylfluorenyl)[(phenyl)(dimethylfluorenyl)triazinylphenyl]dibenzoselenophene; (dimethylfluorenyl)[di(phenyl)triazinylphenyl]dibenzofuran; (dimethylfluorenyl)[di(phenyl)triazinylphenyl]dibenzothiophene; (dimethylfluorenyl)[di(phenyl)triazinylphenyl]dibenzoselenophene; bis(dimethylfluorenyl)[di(phenyl)triazinylphenyl]dibenzofuran; bis(dimethylfluorenyl)[di(phenyl)triazinylphenyl]dibenzothiophene; bis(dimethylfluorenyl)[di(phenyl)triazinylphenyl]dibenzoselenophene; (dimethylfluorenyl)[(phenyl)(terphenylyl)triazinylphenyl]dibenzofuran; (dimethylfluorenyl)[(phenyl)(terphenylyl)triazinylphenyl]dibenzothiophene; (dimethylfluorenyl)[(phenyl)(terphenylyl)triazinylphenyl]dibenzoselenophene; (spirocyclopentanefluorenyl)[di(phenyl)triazinylphenyl]dibenzofuran; (spirocyclopentanefluorenyl)[di(phenyl)triazinylphenyl]dibenzothiophene; (spirocyclopentanefluorenyl)[di(phenyl)triazinylphenyl]dibenzoselenophene; (diphenylfluorenyl) [di(phenyl)triazinylphenyl]dibenzofuran; (diphenylfluorenyl)[di(phenyl)triazinylphenyl]dibenzothiophene; (diphenylfluorenyl)[di(phenyl)triazinylphenyl]dibenzoselenophene; (spirobifluorenyl)[di(phenyl)triazinylphenyl]dibenzofuran; (spirobifluorenyl)[di(phenyl)triazinylphenyl]dibenzothiophene; (spirobifluorenyl)[di(phenyl)triazinylphenyl]dibenzoselenophene; (phenyl)(dimethylfluorenyl)[di(phenyl)triazinylphenyl]dibenzofuran; (phenyl)(dimethylfluorenyl)[di(phenyl)triazinylphenyl]dibenzothiophene; (phenyl)(dimethylfluorenyl)[di(phenyl)triazinylphenyl]dibenzoselenophene; (phenyl)(dimethylfluorenyl)[di(phenyl)triazinylphenyl]dibenzofuran; (phenyl)(dimethylfluorenyl)[di(phenyl)triazinylphenyl]dibenzothiophene; (phenyl)(dimethylfluorenyl)[di(phenyl)triazinylphenyl]dibenzoselenophene; [di(methyl)(phenyl)fluorenyl][di(phenyl)triazinylphenyl]dibenzofuran; [di(methyl)(phenyl)fluorenyl][di(phenyl)triazinylphenyl]dibenzothiophene; [di(methyl)(phenyl)fluorenyl][di(phenyl)triazinylphenyl]dibenzoselenophene; [(dimethyl-d₆)fluorenyl][di(phenyl)triazinylphenyl]dibenzofuran; [(dimethyl-d₆)fluorenyl][di(phenyl)triazinylphenyl]dibenzothiophene; [(dimethyl-d₆)fluorenyl][di(phenyl)triazinylphenyl]dibenzoselenophene; (dimethylfluorenyl)[di(phenyl)triazinylphenyl]dibenzofuran; (dimethylfluorenyl)[di(phenyl)triazinylphenyl]dibenzothiophene; (dimethylfluorenyl)[di(phenyl)triazinylphenyl]dibenzoselenophene; (dimethylfluorenyl)[di(phenyl)triazinylphenyl]dibenzofuran; (dimethylfluorenyl)[di(phenyl)triazinylphenyl]dibenzothiophene; (dimethylfluorenyl)[di(phenyl)triazinylphenyl]dibenzoselenophene; (dimethylfluorenyl)[di(phenyl)triazinylphenyl]dibenzofuran; (dimethylfluorenyl)[di(phenyl)triazinylphenyl]dibenzothiophene; (dimethylfluorenyl)[di(phenyl)triazinylphenyl]dibenzoselenophene; (dimethylfluorenyl)[di(phenyl)triazinylphenyl]dibenzofuran; (dimethylfluorenyl)[di(phenyl)triazinylphenyl]dibenzothiophene; (dimethylfluorenyl)[di(phenyl)triazinylphenyl]dibenzoselenophene; (phenyl)(dimethylfluorenyl)[di(phenyl)triazinylphenyl]dibenzofuran; (phenyl)(dimethylfluorenyl)[di(phenyl)triazinylphenyl]dibenzothiophene; (phenyl)(dimethylfluorenyl)[di(phenyl)triazinylphenyl]dibenzoselenophene; (phenyl)(dimethylfluorenyl)[di(phenyl)triazinylphenyl]dibenzofuran; (phenyl)(dimethylfluorenyl)[di(phenyl)triazinylphenyl]dibenzothiophene; (phenyl)(dimethylfluorenyl)[di(phenyl)triazinylphenyl]dibenzoselenophene; (dimethylfluorenyl)[di(phenyl)triazinylphenyl]dibenzofuran; (dimethylfluorenyl)[di(phenyl)triazinylphenyl]dibenzothiophene; (dimethylfluorenyl)[di(phenyl)triazinylphenyl]dibenzoselenophene; (dimethylfluorenyl)[(diphenyl-d₁₀)triazinylphenyl]dibenzofuran; (dimethylfluorenyl)[(diphenyl-d₁₀)triazinylphenyl]dibenzothiophene; (dimethylfluorenyl)[(diphenyl-d₁₀)triazinylphenyl]dibenzoselenophene; (dimethylfluorenyl)[(diphenyl-d₁₀)triazinylphenyl-d₄]dibenzofuran; (dimethylfluorenyl)[(diphenyl-d₁₀)triazinylphenyl-d₄]dibenzothiophene; (dimethylfluorenyl)[(diphenyl-d₁₀)triazinylphenyl-d₄]dibenzoselenophene; [(dimethyl-d₆)fluorenyl][(diphenyl-d₁₀)triazinylphenyl]dibenzofuran; [(dimethyl-d₆)fluorenyl][(diphenyl-d₁₀)triazinylphenyl]dibenzothiophene; [(dimethyl-d₆)fluorenyl][(diphenyl-d₁₀)triazinylphenyl]dibenzoselenophene; (dimethylfluorenyl)[(diphenyl-d₁₀)triazinylphenyl-d₄]dibenzofuran; (dimethylfluorenyl)[(diphenyl-d₁₀)triazinylphenyl-d₄]dibenzothiophene; (dimethylfluorenyl)[(diphenyl-d₁₀)triazinylphenyl-d₄]dibenzoselenophene; (terphenylyl)[(phenyl)(biphenylyl)triazinyl]dibenzofuran; (terphenylyl)[(phenyl)(biphenylyl)triazinyl]dibenzothiophene; (terphenylyl)[(phenyl)(biphenylyl)triazinyl]dibenzoselenophene; (terphenylyl)[(phenyl)(biphenylyl)triazinyl]dibenzofuran; (terphenylyl)[(phenyl)(biphenylyl)triazinyl]dibenzothiophene; (terphenylyl)[(phenyl)(biphenylyl)triazinyl]dibenzoselenophene; (terphenylyl)[di(phenyl)triazinyl]dibenzofuran; (terphenylyl)[di(phenyl)triazinyl]dibenzothiophene; (terphenylyl)[di(phenyl)triazinyl]dibenzoselenophene; (terphenylyl)[di(biphenylyl)triazinyl]dibenzofuran; (terphenylyl)[di(biphenylyl)triazinyl]dibenzothiophene; (terphenylyl)[di(biphenylyl)triazinyl]dibenzoselenophene; (terphenylyl)[(phenyl)(biphenylyl)triazinyl]dibenzofuran; (terphenylyl)[(phenyl)(biphenylyl)triazinyl]dibenzothiophene; (terphenylyl)[(phenyl)(biphenylyl)triazinyl]dibenzoselenophene; (terphenylyl)[(phenyl)(biphenylyl)triazinyl]dibenzofuran; (terphenylyl)[(phenyl)(biphenylyl)triazinyl]dibenzothiophene; (terphenylyl)[(phenyl)(biphenylyl)triazinyl]dibenzoselenophene; (terphenylyl)[di(phenyl)triazinyl]dibenzofuran; (terphenylyl)[di(phenyl)triazinyl]dibenzothiophene; (terphenylyl)[di(phenyl)triazinyl]dibenzoselenophene; (terphenylyl)[(phenyl)(dibenzothiophenyl)triazinyl]dibenzofuran; (terphenylyl)[(phenyl)(dibenzothiophenyl)triazinyl]dibenzothiophene; (terphenylyl)[(phenyl)(dibenzothiophenyl)triazinyl]dibenzoselenophene; (terphenylyl)[(phenyl)(dibenzofuranyl)triazinyl]dibenzofuran; (terphenylyl)[(phenyl)(dibenzofuranyl)triazinyl]dibenzothiophene; (terphenylyl)[(phenyl)(dibenzofuranyl)triazinyl]dibenzoselenophene; (terphenylyl)[(phenyl)(dimethylfluorenyl)triazinyl]dibenzofuran; (terphenylyl)[(phenyl)(dimethylfluorenyl)triazinyl]dibenzothiophene; (terphenylyl)[(phenyl)(dimethylfluorenyl)triazinyl]dibenzoselenophene; (terphenylyl)[(phenyl)(dibenzothiophenyl)triazinyl]dibenzofuran; (terphenylyl)[(phenyl)(dibenzothiophenyl)triazinyl]dibenzothiophene; (terphenylyl)[(phenyl)(dibenzothiophenyl)triazinyl]dibenzoselenophene; (terphenylyl)[(phenyl)(dibenzofuranyl)triazinyl]dibenzofuran; (terphenylyl)[(phenyl)(dibenzofuranyl)triazinyl]dibenzothiophene; (terphenylyl)[(phenyl)(dibenzofuranyl)triazinyl]dibenzoselenophene; (terphenylyl)[(phenyl)(dimethylfluorenyl)triazinyl]dibenzofuran; (terphenylyl)[(phenyl)(dimethylfluorenyl)triazinyl]dibenzothiophene; (terphenylyl)[(phenyl)(dimethylfluorenyl)triazinyl]dibenzoselenophene; (terphenylyl)[di(phenyl)triazinylphenyl]dibenzofuran; (terphenylyl)[di(phenyl)triazinylphenyl]dibenzothiophene; (terphenylyl)[di(phenyl)triazinylphenyl]dibenzoselenophene; (terphenylyl)[di(phenyl)triazinylphenyl]dibenzofuran; (terphenylyl)[di(phenyl)triazinylphenyl]dibenzothiophene; (terphenylyl)[di(phenyl)triazinylphenyl]dibenzoselenophene; (terphenyld₁₃)[(diphenyl-d₁₀)triazinylphenyl-d₃]dibenzofuran-d₆; (terphenyld₁₃)[(diphenyl-d₁₀)triazinylphenyl-d₃]dibenzothiophened₆; (terphenyld₁₃)[(diphenyl-d₁₀)triazinylphenyl-d₃]dibenzoselenophene-d₆; (terphenylyl)[(diphenyl-d₁₀)triazinylphenyl]dibenzofuran; (terphenylyl)[(diphenyl-d₁₀)triazinylphenyl]dibenzothiophene; (terphenylyl)[(diphenyl-d₁₀)triazinylphenyl]dibenzoselenophene; (terphenylyl)[(diphenyl-d₁₀)triazinylphenyl]dibenzofuran; (terphenylyl)[(diphenyl-d₁₀)triazinylphenyl]dibenzothiophene; (terphenylyl)[(diphenyl-d₁₀)triazinylphenyl]dibenzoselenophene

### (Group 23A)

di(phenyl)[(phenyl)indoloindolyl]triazine; di(phenyl)[(phenyl)indoloindolyl]pyridine; di(phenyl)[(phenyl)indoloindolyl]pyrimidine; di(phenyl)[(phenyl)indoloindolyl]pyrimidine; di(biphenylyl)[(phenyl)indoloindolyl]triazine; di(phenyl)[(diphenyltriazinyl)indoloindolyl]triazine; di(phenyl)[(phenyl)indoloindolylphenyl]triazine; (phenyl)[(phenyl)indoloindolyl]quinazoline; bis(phenylcarbazolyl)indoloindole; (phenyl)(phenylcarbazolyl)indoloindole; (phenyl)(dibenzofuranyl)indoloindole; (phenyl)(dibenzothiophenyl)indoloindole; (phenyl)(carbazolylphenyl)indoloindole; di(phenyl)(benzothienoindolyl)triazine; (phenyl)(biphenylyl)(benzothienoindolyl)triazine; di(biphenylyl)(benzothienoindolyl)triazine; (phenyl)(carbazolylphenyl)(benzothienoindolyl)triazine; (phenylquinzolyl)benzothienoindole; (phenylcarbazolyl)benzothienoindole; (naphthylcarbazolyl)benzothienoindole; (triphenylenylcarbazolyl)benzothienoindole; (biphenylcarbazolyl)benzothienoindole; (terphenylcarbazolyl)benzothienoindole; (diphenylpyridyl)benzofuroindole; (diphenylpyrimidinyl)benzofuroindole; di(phenyl)(benzofuroindolyl)triazine; (phenyl)(biphenylyl)(benzofuroindolyl)triazine; di(biphenylyl)(benzofuroindolyl)triazine; (biphenylyl)(benzofuroindolyl)quinazoline; (carbazolyl)(benzofuroindolyl)dibenzofuran; (phenylcarbazolyl)benzofuroindole; (naphthylcarbazolyl)benzofuroindole; (terphenylcarbazolyl)benzofuroindole; di(phenyl)(phenylbenzofuroindolyl)triazine; di(phenyl)(carbazolylbenzofuroindolyl)triazine; di(phenyl)bicarbazole; di(biphenylyl)bicarbazole; (phenyl)(biphenylyl)bicarbazole; di(biphenylyl)bicarbazole; (phenyl)(biphenylyl)bicarbazole; di(biphenylyl)bicarbazole; (phenyl)(naphthylphenyl)bicarbazole; (phenyl)(naphthylphenyl)bicarbazole; (phenyl)(naphthyl)bicarbazole; di(naphthyl)bicarbazole; (naphthyl)(biphenylyl)bicarbazole; (naphthyl)(biphenylyl)bicarbazole; (naphthyl)(terphenylyl)bicarbazole; (naphthyl)(naphthylphenyl)bicarbazole; di(naphthyl)bicarbazole; (naphthyl)(phenanthryl)bicarbazole; (naphthyl)(phenylanthryl)bicarbazole; (phenyl)(naphthyl)bicarbazole; di(naphthyl)bicarbazole; (naphthyl)(biphenylyl)bicarbazole; (naphthyl)(biphenylyl)bicarbazole; (naphthyl)(terphenylyl)bicarbazole; (naphthyl)(naphthylphenyl)bicarbazole; di(naphthyl)bicarbazole; (naphthyl)(phenanthryl)bicarbazole; (naphthyl)(phenylanthryl)bicarbazole; (phenyl)(phenanthryl)bicarbazole; (biphenylyl)(phenanthryl)bicarbazole; (biphenylyl)(phenanthryl)bicarbazole; (terphenylyl)(phenanthryl)bicarbazole; (naphthylphenyl)(phenanthryl)bicarbazole; di(phenanthryl)bicarbazole; (phenylanthryl)(phenanthryl)bicarbazole; (phenyl)(naphthyl)bicarbazole; di(naphthyl)bicarbazole; (phenyl)(naphthyl)bicarbazole; di(naphthyl)bicarbazole; (phenyl)(phenanthryl)bicarbazole; di(phenanthryl)bicarbazole; (phenyl)(anthryl)bicarbazole; di(anthryl)bicarbazole; (phenyl)(naphthyl)bicarbazole; di(naphthyl)bicarbazole; (phenyl)(naphthyl)bicarbazole; di(naphthyl)bicarbazole; (phenyl)(phenanthryl)bicarbazole; di(phenanthryl)bicarbazole; (phenyl)(anthryl)bicarbazole; di(anthryl)bicarbazole; (phenyl)(naphthyl)bicarbazole; di(naphthyl)bicarbazole; (phenyl)(naphthyl)bicarbazole; di(naphthyl)bicarbazole; (phenyl)(phenanthryl)bicarbazole; di(phenanthryl)bicarbazole; (phenyl)(naphthyl)bicarbazole; di(naphthyl)bicarbazole; (phenyl)(naphthyl)bicarbazole; (phenyl)(naphthyl)bicarbazole; di(naphthyl)bicarbazole; (phenyl)(naphthyl)bicarbazole; (phenyl)(phenanthryl)bicarbazole; di(phenanthryl)bicarbazole; (phenyl)(phenanthryl)bicarbazole; (phenyl)(anthryl)bicarbazole; (phenyl)(naphthyl)bicarbazole; (phenyl)(naphthyl)bicarbazole; di(naphthyl)bicarbazole; (phenyl)(naphthyl)bicarbazole; (phenyl)(phenanthryl)bicarbazole; (phenyl)(anthryl)bicarbazole; (phenyl)(naphthyl)bicarbazole; (phenyl)(naphthyl)bicarbazole; di(naphthyl)bicarbazole; (phenyl)(naphthyl)bicarbazole; (phenyl)(naphthyl)bicarbazole; di(naphthyl)bicarbazole; (phenyl)(phenanthryl)bicarbazole; (phenyl)(phenanthryl)bicarbazole; di(phenanthryl)bicarbazole; (phenyl)(anthryl)bicarbazole; (phenyl)(anthryl)bicarbazole; di(anthryl)bicarbazole; (phenyl)(pyrenyl)bicarbazole; (phenyl)(triphenylenyl)bicarbazole; (biphenylyl)(dibenzofuranyl)carbazole; (biphenylyl)(dibenzofuranyl)carbazole; (terphenylyl)(dibenzofuranyl)carbazole; (naphthylphenyl)(dibenzofuranyl)carbazole; (naphthylphenyl)(dibenzofuranyl)carbazole; (naphthyl)(dibenzofuranyl)carbazole; (phenanthryl)(dibenzofuranyl)carbazole; (triphenylenyl)(dibenzofuranyl)carbazole; (naphthyl)(dibenzofuranyl)carbazole; (biphenylyl)(dibenzothiophenyl)carbazole; (terphenylyl)(dibenzothiophenyl)carbazole; (naphthylphenyl)(dibenzothiophenyl)carbazole; (naphthylphenyl)(dibenzothiophenyl)carbazole; (naphthyl)(dibenzothiophenyl)carbazole; (phenanthryl)(dibenzothiophenyl)carbazole; (triphenylenyl)(dibenzothiophenyl)carbazole; (naphthyl)(dibenzothiophenyl)carbazole; (phenyl)(phenylpyridyl)bicarbazole; (phenylpyridyl)(dibenzofuranyl)carbazole; (phenylpyridyl)(dibenzothiophenyl)carbazole; (phenyl)(diphenylpyrimidinyl)bicarbazole; (diphenylpyrimidinyl)(dibenzofuranyl)carbazole; (diphenylpyrimidinyl)(dibenzothiophenyl)carbazole; (phenyl)(diphenyltriazinyl)bicarbazole; (diphenyltriazinyl)(dibenzofuranyl)carbazole; (diphenyltriazinyl)(dibenzothiophenyl)carbazole; (phenyl)(phenylquinzolyl)bicarbazole; (phenylquinzolyl)(dibenzofuranyl)carbazole; (phenylquinzolyl)(dibenzothiophenyl)carbazole

### (Group 24A)

(phenyl)(biphenylyl)[(phenyl)indolocarbazolyl]triazine; (phenyl)(biphenylyl)[(phenyl)indolocarbazolyl]triazine; di(phenyl)[(biphenylyl)indolocarbazolyl]triazine; di(phenyl)[(biphenylyl)indolocarbazolyl]triazine; di(phenyl)[di(phenyl)indolocarbazolyl]triazine; di(phenyl)[di(phenyl)indolocarbazolyl]triazine; di(phenyl)[di(phenyl)indolocarbazolyl]triazine; di(phenyl)[(phenyl)(naphthyl)indolocarbazolyl]triazine; di(phenyl)[(phenyl)(quinolyl)indolocarbazolyl]triazine; di(phenyl)[(phenyl)(naphthyl)indolocarbazolyl]triazine; (phenyl)(terphenylyl)[(phenyl)indolocarbazolyl]triazine; (phenyl)(terphenylyl)[(phenyl)indolocarbazolyl]triazine; (phenyl)(terphenylyl)[(phenyl)indolocarbazolyl]triazine; (phenyl)(terphenylyl)[(phenyl)indolocarbazolyl]triazine; di(phenyl)[(terphenylyl)indolocarbazolyl]triazine; di(phenyl)[(terphenylyl)indolocarbazolyl]triazine; di(phenyl)[(terphenylyl)indolocarbazolyl]triazine; di(phenyl)[(terphenylyl)indolocarbazolyl]triazine; di(biphenylyl)[(phenyl)indolocarbazolyl]triazine; di(biphenylyl)[(phenyl)indolocarbazolyl]triazine; di(biphenylyl)[(phenyl)indolocarbazolyl]triazine; di(phenyl)[(phenyl)(biphenylyl)indolocarbazolyl]triazine; di(phenyl)[(phenyl)(biphenylyl)indolocarbazolyl]triazine; di(phenyl)[tri(phenyl)indolocarbazolyl]triazine; di(phenyl)[tri(phenyl)indolocarbazolyl]triazine; di(phenyl)[(phenyl)(biphenylyl)indolocarbazolyl]triazine; di(phenyl)[(phenyl)(biphenylyl)indolocarbazolyl]triazine; di(phenyl)[(phenyl)di(pyridyl)indolocarbazolyl]triazine; di(phenyl)[(phenyl)di(methyl)indolocarbazolyl]triazine; di(phenyl)[(phenyl)di(butyl)indolocarbazolyl]triazine; di(phenyl)[(phenyl)di(tert-butyl)indolocarbazolyl]triazine; (phenyl)(biphenylyl)[(phenyl)di(butyl)indolocarbazolyl]triazine; (phenyl)(biphenylyl)[(phenyl)di(tert-butyl)indolocarbazolyl]triazine; (phenyl)(terphenylyl)[(phenyl)di(butyl)indolocarbazolyl]triazine; (phenyl)(terphenylyl)[(phenyl)di(tert-butyl)indolocarbazolyl]triazine; (phenyl)(biphenylyl)[(phenyl)di(butyl)indolocarbazolyl]triazine; (phenyl)(biphenylyl)[(phenyl)di(tert-butyl)indolocarbazolyl]triazine; (phenyl)(terphenylyl)[(phenyl)di(tert-butyl)indolocarbazolyl]triazine; (phenyl)(terphenylyl)[(phenyl)di(butyl)indolocarbazolyl]triazine; (biphenylyl)(terphenylyl)[(phenyl)di(butyl)indolocarbazolyl]triazine; (phenyl)(biphenylyl)[(phenyl)indolocarbazolyl]triazine; (phenyl)(biphenylyl)[(phenyl)indolocarbazolyl]triazine; di(phenyl)[(biphenylyl)indolocarbazolyl]triazine; di(phenyl)[(biphenylyl)indolocarbazolyl]triazine; di(phenyl)[di(phenyl)indolocarbazolyl]triazine; di(phenyl)[di(phenyl)indolocarbazolyl]triazine; di(phenyl)[di(phenyl)indolocarbazolyl]triazine; di(phenyl)[(phenyl)(naphthyl)indolocarbazolyl]triazine; di(phenyl)[(phenyl)(quinolyl)indolocarbazolyl]triazine; di(phenyl)[(phenyl)(naphthyl)indolocarbazolyl]triazine; (phenyl)(terphenylyl)[(phenyl)indolocarbazolyl]triazine; (phenyl)(terphenylyl)[(phenyl)indolocarbazolyl]triazine; (phenyl)(terphenylyl)[(phenyl)indolocarbazolyl]triazine; (phenyl)(terphenylyl)[(phenyl)indolocarbazolyl]triazine; di(phenyl)[(terphenylyl)indolocarbazolyl]triazine; di(phenyl)[(terphenylyl)indolocarbazolyl]triazine; di(phenyl)[(terphenylyl)indolocarbazolyl]triazine; di(biphenylyl)[(phenyl)indolocarbazolyl]triazine; di(biphenylyl)[(phenyl)indolocarbazolyl]triazine; di(biphenylyl)[(phenyl)indolocarbazolyl]triazine; di(biphenylyl)[(phenyl)indolocarbazolyl]triazine; di(phenyl)[(phenyl)(biphenylyl)indolocarbazolyl]triazine; di(phenyl)[(phenyl)(biphenylyl)indolocarbazolyl]triazine; di(phenyl)[tri(phenyl)indolocarbazolyl]triazine; di(phenyl)[tri(phenyl)indolocarbazolyl]triazine; di(phenyl)[di(phenyl)(biphenylyl)indolocarbazolyl]triazine; di(phenyl)[di(phenyl)(biphenylyl)indolocarbazolyl]triazine; di(phenyl)[(phenyl)di(pyridyl)indolocarbazolyl]triazine; di(phenyl)[(phenyl)di(methyl)indolocarbazolyl]triazine; di(phenyl)[(phenyl)di(butyl)indolocarbazolyl]triazine; di(phenyl)[(phenyl)di(tert-butyl)indolocarbazolyl]triazine; (phenyl)(biphenylyl)[(phenyl)di(butyl)indolocarbazolyl]triazine; (phenyl)(biphenylyl)[(phenyl)di(tert-butyl)indolocarbazolyl]triazine; (phenyl)(terphenylyl)[(phenyl)di(butyl)indolocarbazolyl]triazine; (phenyl)(terphenylyl)[(phenyl)di(tert-butyl)indolocarbazolyl]triazine; (phenyl)(biphenylyl)[(phenyl)di(butyl)indolocarbazolyl]triazine; (phenyl)(biphenylyl)[(phenyl)di(tert-butyl)indolocarbazolyl]triazine; (phenyl)(terphenylyl)[(phenyl)di(tert-butyl)indolocarbazolyl]triazine; (phenyl)(terphenylyl)[(phenyl)di(butyl)indolocarbazolyl]triazine; (biphenylyl)(terphenylyl)[(phenyl)di(butyl)indolocarbazolyl]triazine; (phenyl)(biphenylyl)[(phenyl)indolocarbazolyl]triazine; (phenyl)(biphenylyl)[(phenyl)indolocarbazolyl]triazine; di(phenyl)[(biphenylyl)indolocarbazolyl]triazine; di(phenyl)[(biphenylyl)indolocarbazolyl]triazine; di(phenyl)[di(phenyl)indolocarbazolyl]triazine; di(phenyl)[di(phenyl)indolocarbazolyl]triazine; di(phenyl)[di(phenyl)indolocarbazolyl]triazine; di(phenyl)[(phenyl)(naphthyl)indolocarbazolyl]triazine; di(phenyl)[(phenyl)(quinolyl)indolocarbazolyl]triazine; di(phenyl)[(phenyl)(naphthyl)indolocarbazolyl]triazine; (phenyl)(terphenylyl)[(phenyl)indolocarbazolyl]triazine; (phenyl)(terphenylyl)[(phenyl)indolocarbazolyl]triazine; (phenyl)(terphenylyl)[(phenyl)indolocarbazolyl]triazine; (phenyl)(terphenylyl)[(phenyl)indolocarbazolyl]triazine; di(phenyl)[(terphenylyl)indolocarbazolyl]triazine; di(phenyl)[(terphenylyl)indolocarbazolyl]triazine; di(phenyl)[(terphenylyl)indolocarbazolyl]triazine; di(phenyl)[(terphenylyl)indolocarbazolyl]triazine; di(biphenylyl)[(phenyl)indolocarbazolyl]triazine; di(biphenylyl)[(phenyl)indolocarbazolyl]triazine; di(biphenylyl)[(phenyl)indolocarbazolyl]triazine; di(phenyl)[(phenyl)(biphenylyl)indolocarbazolyl]triazine; di(phenyl)[(phenyl)(biphenylyl)indolocarbazolyl]triazine; di(phenyl)[tri(phenyl)indolocarbazolyl]triazine; di(phenyl)[tri(phenyl)indolocarbazolyl]triazine; di(phenyl)[di(phenyl)(biphenylyl)indolocarbazolyl]triazine; di(phenyl)[di(phenyl)(biphenylyl)indolocarbazolyl]triazine; di(phenyl)[(phenyl)di(pyridyl)indolocarbazolyl]triazine; di(phenyl)[(phenyl)di(methyl)indolocarbazolyl]triazine; di(phenyl)[(phenyl)di(butyl)indolocarbazolyl]triazine; di(phenyl)[(phenyl)di(tert-butyl)indolocarbazolyl]triazine; (phenyl)(biphenylyl)[(phenyl)di(butyl)indolocarbazolyl]triazine; (phenyl)(biphenylyl)[(phenyl)di(tert-butyl)indolocarbazolyl]triazine; (phenyl)(terphenylyl)[(phenyl)di(butyl)indolocarbazolyl]triazine; (phenyl)(terphenylyl)[(phenyl)di(tert-butyl)indolocarbazolyl]triazine; (phenyl)(biphenylyl)[(phenyl)di(butyl)indolocarbazolyl]triazine; (phenyl)(biphenylyl)[(phenyl)di(tert-butyl)indolocarbazolyl]triazine; (phenyl)(terphenylyl)[(phenyl)di(butyl)indolocarbazolyl]triazine; (phenyl)(terphenylyl)[(phenyl)di(tert-butyl)indolocarbazolyl]triazine; (biphenylyl)(terphenylyl)[(phenyl)di(tert-butyl)indolocarbazolyl]triazine; (phenyl)(biphenylyl)[(phenyl)indolocarbazolyl]triazine; (phenyl)(biphenylyl)[(phenyl)indolocarbazolyl]triazine; di(phenyl)[(biphenylyl)indolocarbazolyl]triazine; di(phenyl)[(biphenylyl)indolocarbazolyl]triazine; di(phenyl)[tri(phenyl)indolocarbazolyl]triazine; di(phenyl)[(phenyl)di(butyl)indolocarbazolyl]triazine; (phenyl)(terphenylyl)[(phenyl)indolocarbazolyl]triazine; (phenyl)(terphenylyl)[(phenyl)indolocarbazolyl]triazine; (phenyl)(terphenylyl)[(phenyl)indolocarbazolyl]triazine; (phenyl)(terphenylyl)[(phenyl)indolocarbazolyl]triazine; di(biphenylyl)[(phenyl)indolocarbazolyl]triazine; di(biphenylyl)[(phenyl)indolocarbazolyl]triazine; (phenyl)(biphenylyl)[(phenyl)indolocarbazolyl]triazine; (phenyl)(biphenylyl)[(phenyl)indolocarbazolyl]triazine; (phenyl)(terphenylyl)[(phenyl)indolocarbazolyl]triazine; (phenyl)(terphenylyl)[(phenyl)indolocarbazolyl]triazine; (phenyl)(terphenylyl)[(phenyl)indolocarbazolyl]triazine; (phenyl)(terphenylyl)[(phenyl)indolocarbazolyl]triazine; di(biphenylyl)[(phenyl)indolocarbazolyl]triazine; di(biphenylyl)[(phenyl)indolocarbazolyl]triazine; (phenyl)(biphenylyl)[(phenyl)indolocarbazolyl]triazine; (phenyl)(biphenylyl)[(phenyl)indolocarbazolyl]triazine; di(phenyl)[(biphenylyl)indolocarbazolyl]triazine; di(phenyl)[(biphenylyl)indolocarbazolyl]triazine; di(phenyl)[tri(phenyl)indolocarbazolyl]triazine; di(phenyl)[(phenyl)di(butyl)indolocarbazolyl]triazine; (phenyl)(terphenylyl)[(phenyl)indolocarbazolyl]triazine; (phenyl)(terphenylyl)[(phenyl)indolocarbazolyl]triazine; (phenyl)(terphenylyl)[(phenyl)indolocarbazolyl]triazine; (phenyl)(terphenylyl)[(phenyl)indolocarbazolyl]triazine; di(biphenylyl)[(phenyl)indolocarbazolyl]triazine; di(biphenylyl)[(phenyl)indolocarbazolyl]triazine; di(terphenylyl)[(phenyl)indolocarbazolyl]triazine; di(terphenylyl)[(phenyl)indolocarbazolyl]triazine; di(terphenylyl)[(phenyl)indolocarbazolyl]triazine; di(terphenylyl)[(phenyl)indolocarbazolyl]triazine; (phenyl)(biphenylyl)[(phenyl)(dibenzofuranyl)indolocarbazolyl]triazine; (phenyl)(biphenylyl)[(phenyl)(dibenzothiophenyl)indolocarbazolyl]triazine; (phenyl)(terphenylyl)[(phenyl)(dibenzofuranyl)indolocarbazolyl]triazine; (phenyl)(terphenylyl)[(phenyl)(dibenzothiophenyl)indolocarbazolyl]triazine; di(phenyl)[(phenyl)indolocarbazolyl]triazine; di(phenyl)[(phenyl)indolocarbazolyl]triazine; di(phenyl)[(phenyl)indolocarbazolyl]triazine; di(phenyl)[(phenyl)indolocarbazolyl]triazine; di(phenyl)[(phenyl)indolocarbazolyl]triazine; di(biphenylyl)bicarbazole; (biphenylyl)(phenanthryl)bicarbazole; (biphenylyl)(anthryl)bicarbazole; (biphenylyl)(naphthylphenyl)bicarbazole; (biphenylyl)(naphthylphenyl)bicarbazole; (biphenylyl)(terphenylyl)bicarbazole; (biphenylyl)(anthrylphenyl)bicarbazole; (biphenylyl)(naphthylphenyl)bicarbazole; (biphenylyl)(anthrylphenyl)bicarbazole; (biphenylyl)(naphthylphenyl)bicarbazole; (biphenylyl)(terphenylyl)bicarbazole; (biphenylyl)(terphenylyl)bicarbazole; (biphenylyl)(terphenylyl)bicarbazole; (biphenylyl)(terphenylyl)bicarbazole; (biphenylyl)(naphthylphenyl)bicarbazole; (biphenylyl)(naphthylphenyl)bicarbazole; (biphenylyl)(phenanthrylphenyl)bicarbazole; (biphenylyl)(terphenylyl)bicarbazole; (biphenylyl)(naphthylbiphenylyl)bicarbazole; (biphenylyl)(naphthylphenyl)bicarbazole; (biphenylyl)(naphthylphenyl)bicarbazole; (biphenylyl)(phenanthrylphenyl)bicarbazole; (biphenylyl)(terphenylyl)bicarbazole; (biphenylyl)(naphthylbiphenylyl)bicarbazole; (biphenylyl)(naphthylphenyl)bicarbazole; (biphenylyl)(naphthylphenyl)bicarbazole; (biphenylyl)(phenanthrylphenyl)bicarbazole; (phenyl)(biphenylyl)bicarbazole; (phenyl)(naphthylphenyl)bicarbazole; (phenyl)(naphthylphenyl)bicarbazole; (phenyl)(phenanthrylphenyl)bicarbazole; (phenyl)(terphenylyl)bicarbazole; (phenyl)(terphenylyl)bicarbazole; (phenyl)(terphenylyl)bicarbazole; (phenyl)(naphthylbiphenylyl)bicarbazole; (phenyl)(naphthylbiphenylyl)bicarbazole; (naphthyl)(biphenylyl)bicarbazole; di(biphenylyl)bicarbazole; di(biphenylyl)bicarbazole; di(biphenylyl)bicarbazole; (biphenylyl)(phenanthryl)bicarbazole; (biphenylyl)(anthryl)bicarbazole; (biphenylyl)(naphthylphenyl)bicarbazole; (biphenylyl)(naphthylphenyl)bicarbazole; (biphenylyl)(terphenylyl)bicarbazole; (biphenylyl)(anthrylphenyl)bicarbazole; (biphenylyl)(naphthylphenyl)bicarbazole; (biphenylyl)(anthrylphenyl)bicarbazole; (biphenylyl)(naphthylphenyl)bicarbazole; (biphenylyl)(terphenylyl)bicarbazole; (biphenylyl)(terphenylyl)bicarbazole; (biphenylyl)(terphenylyl)bicarbazole; (biphenylyl)(naphthylbiphenylyl)bicarbazole; (biphenylyl)(naphthylphenyl)bicarbazole; (biphenylyl)(naphthylphenyl)bicarbazole; (biphenylyl)(phenanthrylphenyl)bicarbazole; (biphenylyl)(terphenylyl)bicarbazole; (biphenylyl)(naphthylbiphenylyl)bicarbazole; (biphenylyl)(naphthylphenyl)bicarbazole; (biphenylyl)(naphthylphenyl)bicarbazole; (biphenylyl)(phenanthrylphenyl)bicarbazole; (biphenylyl)(terphenylyl)bicarbazole; (biphenylyl)(naphthylbiphenylyl)bicarbazole; (biphenylyl)(naphthylphenyl)bicarbazole; (biphenylyl)(naphthylphenyl)bicarbazole; (biphenylyl)(phenanthrylphenyl)bicarbazole; (phenyl)(biphenylyl)bicarbazole; (phenyl)(naphthylphenyl)bicarbazole; (phenyl)(naphthylphenyl)bicarbazole; (phenyl)(phenanthrylphenyl)bicarbazole; (phenyl)(terphenylyl)bicarbazole; (phenyl)(terphenylyl)bicarbazole; (phenyl)(terphenylyl)bicarbazole; (phenyl)(naphthylbiphenylyl)bicarbazole; (phenyl)(naphthylbiphenylyl)bicarbazole; di(biphenylyl)bicarbazole; (biphenylyl)(phenanthryl)bicarbazole; (biphenylyl)(anthryl)bicarbazole; (biphenylyl)(naphthylphenyl)bicarbazole; (biphenylyl)(naphthylphenyl)bicarbazole; (biphenylyl)(terphenylyl)bicarbazole; (biphenylyl)(anthrylphenyl)bicarbazole; (biphenylyl)(naphthylphenyl)bicarbazole; (biphenylyl)(anthrylphenyl)bicarbazole; (biphenylyl)(naphthylphenyl)bicarbazole; (biphenylyl)(terphenylyl)bicarbazole; (biphenylyl)(terphenylyl)bicarbazole; (biphenylyl)(terphenylyl)bicarbazole; (biphenylyl)(naphthylbiphenylyl)bicarbazole; (biphenylyl)(naphthylphenyl)bicarbazole; (biphenylyl)(naphthylphenyl)bicarbazole; (biphenylyl)(phenanthrylphenyl)bicarbazole; (naphthyl)(biphenylyl)bicarbazole; di(biphenylyl)bicarbazole; di(biphenylyl)bicarbazole; di(biphenylyl)bicarbazole; (biphenylyl)(phenanthryl)bicarbazole; (biphenylyl)(anthryl)bicarbazole; (biphenylyl)(naphthylphenyl)bicarbazole; (biphenylyl)(naphthylphenyl)bicarbazole; (biphenylyl)(terphenylyl)bicarbazole; (biphenylyl)(anthrylphenyl)bicarbazole; (biphenylyl)(naphthylphenyl)bicarbazole; (biphenylyl)(anthrylphenyl)bicarbazole; (biphenylyl)(naphthylphenyl)bicarbazole; (biphenylyl)(terphenylyl)bicarbazole; (biphenylyl)(terphenylyl)bicarbazole; (biphenylyl)(terphenylyl)bicarbazole; (biphenylyl)(naphthylbiphenylyl)bicarbazole; (biphenylyl)(naphthylphenyl)bicarbazole; (biphenylyl)(naphthylphenyl)bicarbazole; (biphenylyl)(phenanthrylphenyl)bicarbazole; (biphenylyl)(terphenylyl)bicarbazole; (biphenylyl)(naphthylbiphenylyl)bicarbazole; (biphenylyl)(naphthylphenyl)bicarbazole; (biphenylyl)(naphthylphenyl)bicarbazole; (biphenylyl)(phenanthrylphenyl)bicarbazole; (biphenylyl)(terphenylyl)bicarbazole; (biphenylyl)(naphthylbiphenylyl)bicarbazole; (biphenylyl)(naphthylphenyl)bicarbazole; (biphenylyl)(naphthylphenyl)bicarbazole; (biphenylyl)(phenanthrylphenyl)bicarbazole; (phenyl)(biphenylyl)bicarbazole; (phenyl)(naphthylphenyl)bicarbazole; (phenyl)(naphthylphenyl)bicarbazole; (phenyl)(phenanthrylphenyl)bicarbazole; (phenyl)(terphenylyl)bicarbazole; (phenyl)(terphenylyl)bicarbazole; (phenyl)(terphenylyl)bicarbazole; (phenyl)(naphthylbiphenylyl)bicarbazole; (phenyl)(naphthylbiphenylyl)bicarbazole

### (Group 25A)

di(bipyridinyl)triphenylene; di(bipyridinyl)triphenylene; bis(dipyridinylphenyl)phenylpyrimidine; (phenylquinquephenylyl)diphenylpyrimidine; (phenylquarterphenylyl)diphenylpyrimidine; bis[(biphenylpyrimidinyl)phenyl]ether; di(terphenylyl)sulfone; bis(diphenylterphenylyl)sulfone; phenyl(diphenylquinquephenylyl)sulfone; di(spirobifluorenyl)ketone; bis(diphenylfluorenyl)ketone; bis(dimethylfluorenyl)naphthyltriazine; bis(dimethylfluorenyl)biphenyltriazine; bis(dimethylfluorenyl)terphenyltriazine; biphenyl(spirobifluorenyl)phenylpyridine; biphenyl(spirobifluorenyl)phenylpyridine; biphenyl(spirobifluorenyl)naphthylpyridine; di(terphenylyl)(spirobifluorenyl)pyridine; biphenyl(spirobifluorenyl)phenylpyridine; biphenyl(spirobifluorenyl)phenylpyridine; biphenyl(spirobifluorenyl)naphthylpyridine; di(terphenylyl)(spirobifluorenyl)pyridine; biphenyl(spirobifluorenyl)phenylpyridine; biphenyl(spirobifluorenyl)phenylpyridine; biphenyl(spirobifluorenyl)naphthylpyridine; biphenyl(spirobifluorenyl)phenylpyrimidine; biphenyl(spirobifluorenyl)phenylpyrimidine; di(biphenylyl)(spirobifluorenyl)phenylpyrimidine; biphenyl(spirobifluorenyl)naphthylpyrimidine; terphenyl(spirobifluorenyl)phenylpyrimidine; biphenyl(spirobifluorenyl)phenylpyrimidine; biphenyl(spirobifluorenyl)phenylpyrimidine; biphenyl(spirobifluorenyl)naphthylpyrimidine; biphenyl(spirobifluorenyl)naphthylpyrimidine; terphenyl(spirobifluorenyl)phenylpyrimidine; biphenyl(spirobifluorenyl)phenyltriazine; biphenyl(spirobifluorenyl)phenyltriazine; (biphenyl-d₁)(spirobifluorenyl)phenyltriazine; (biphenyl-d₅)(spirobifluorenyl)phenyltriazine; biphenyl(spirobifluorenyl)(phenyl-d₅)triazine; terphenyl(spirobifluorenyl)phenyltriazine; terphenyl(spirobifluorenyl)naphthyltriazine; biphenyl(spirobifluorenyl)quinazoline; biphenyl(spirobifluorenyl)quinazoline; terphenyl(spirobifluorenyl)quinazoline; terphenyl(spirobifluorenyl)quinazoline; (biphenyl-d₁)(spirobifluorenyl)quinazoline; terphenyl(spirobifluorenyl)quinazoline; di(terphenylyl)diboraoxanaphthoanthracene; di(terphenylyl)diboraoxanaphthoanthracene; [(triphenylenyl)biphenyl]diboraoxanaphthoanthracene; (quarterphenylyl)diboraoxanaphthoanthracene; (phenylquarterphenylyl)diboraoxanaphthoanthracene; di(dibenzofuranyl)diboraoxanaphthoanthracene; di(dibenzofuranyl)diboraoxanaphthoanthracene; di(biphenylyl)(dimethylfluorenyl)triazine; (terphenylyl)(biphenylyl)(dimethylfluorenyl)triazine; di(terphenylyl)(dimethylfluorenyl)triazine; (biphenylyl)(diphenylfluorenyl)phenyltriazine; (biphenylyl)(diphenylfluorenyl)phenyltriazine; bis(diphenylphosphoryl)dibenzothiophene; bis(diphenylphosphoryl)dibenzofuran; diphenyl(triphenylenylterphenylyl)amine; diphenyl(quinquephenylyl)amine; diphenyl(phenylquinquephenylyl)amine; diphenyl[(spirobifluorenyl)biphenyl]amine; bis[(diphenylamino)phenyl]dibenzofuran; [(triphenylenyl)terphenyl]indole; tris(indolylphenyl)benzene; (diphenylquinquephenylyl)carbazole; [(triphenylenyl)terphenyl]carbazole; [(triphenylenyl)biphenyl]carbazole; di(carbazolyl)quarterbenzene; (carbazolylbiphenylyl)[di(biphenylyl)]amine; terphenylbicarbazole; quarterphenylbicarbazole; bis(diphenylcarbazolyl)benzene; di(carbazolyl)terbenzene; di(carbazolyl)quarterbenzene; di(carbazolyl)quarterbenzene; di(carbazolyl)spirobifluorene; [(triphenylenyl)biphenyl]carbazolyldibenzothiophene; di(carbazolyl)dibenzothiophene; di(carbazolyl)dibenzofuran; di(carbazolyl)(diphenylfluorene); di(carbazolyl)(biphenylyl)carbazole; (quarterphenylyl)bicarbazole; bis(bicarbazolyl)benzene; bis(bicarbazolyl)benzene; bis(bicarbazolyl)biphenyl; bis[di(carbazolyl)carbazolyl]benzene; bis[di(carbazolyl)carbazolyl]biphenyl; tris(carbazolyl)benzene; dimethyl(phenylquarterphenylyl)acridone; (phenylquarterphenylyl)phenoxazine; (phenylquarterphenylyl)phenothiazine; phenyl(phenylquarterphenylyl)fenadine; dimethyl[(terphenylyl)carbazolyl]acridone; dimethyl[(terphenylyl)carbazolyl]phenoxazine; dimethyl[(terphenylyl)carbazolyl]phenothiazine; phenyl(spirobifluorenyl)fenadine; bis(dimethylacridinyl)terbenzene; di(phenoxazinyl)terbenzene; di(phenoxazinyl)biphenyl; di(phenothiazinyl)terbenzene; bis(dimethylacridinyl)biphenyl; di(biphenylyl)bicarbazole; di(biphenylyl)bicarbazole; phenyl(terphenylyl)bicarbazole; di(biphenylyl)bicarbazole-d₂; phenyl(triphenylenyl)bicarbazole; biphenyl(terphenylyl)bicarbazole; diphenyltercarbazole; phenyl(dibenzothiophenyl)bicarbazole; phenyl(dimethylfluorenyl)bicarbazole; di(biphenylyl)bicarbazole; phenyl(triphenylenyl)bicarbazole; di(biphenylyl)bicarbazole-d₂; di(biphenylyl)bicarbazole-d₂; phenyl(terphenylyl)bicarbazole; phenyl(triphenylenyl)bicarbazole; di(biphenylyl)bicarbazole; diphenyltercarbazole; phenyl(triphenylenyl)bicarbazole-d₁; di(biphenylyl)bicarbazole; phenyl(triphenylenyl)bicarbazole; di(biphenylyl)bicarbazole; phenyl(triphenylenyl)bicarbazole; phenyl(dibenzofuranyl)bicarbazole; phenyl(terphenylyl)bicarbazole-d₂; di(biphenylyl)bicarbazole; phenyl(triphenylenyl)bicarbazole; (phenylcarbazolyl)[(triphenylenyl)carbazoyl]benzene; (phenylcarbazolyl)[(biphenylyl)carbazoyl]benzene; (phenylcarbazolyl)[(biphenylyl)carbazoyl]benzene; (phenylcarbazolyl)[(terphenylyl)carbazolyl]benzene; bis(phenylcarbazolyl)biphenyl; bis[(biphenylyl)carbazoyl]biphenyl; (phenylcarbazolyl)[(triphenylenyl)carbazoyl]biphenyl; (phenylcarbazolyl)[(terphenylyl)carbazolyl]biphenyl; bis(phenylcarbazolyl)biphenyl-d₂; terphenyl(dimethylfluorenyl)carbazole; quarterphenyl(dimethylfluorenyl)carbazole; terphenyl(dimethylfluorenyl)carbazole; terphenyl(diphenylfluorenyl)carbazole; quarterphenyl(dibenzothiophenyl)carbazole; terphenyl(dibenzofuranyl)carbazole; biphenyl(dibenzofuranyl)bicarbazole; terphenyl(dibenzothiophenyl)carbazole; di(biphenylyl)indolocarbazole; di(biphenylyl)indolocarbazole; phenyl(terphenylyl)indolocarbazole-d₂; bis(phenylindolocarbazolyl)benzene; di(biphenylyl)indolocarbazole; phenyl(terphenylyl)indolocarbazole; phenyl(terphenylyl)indolocarbazole-d₁; bis(phenylindolocarbazolyl)benzene; bis(phenylindolocarbazolyl)biphenyl; phenyl(dinaphthylphenyl)indolocarbazole; phenyl(spirobifluorenyl)indolocarbazole; di(biphenylyl)indolocarbazole; bis(phenylindolocarbazolyl)biphenyl; phenyl(terphenylyl)indolocarbazole; di(biphenylyl)indolocarbazole; bis(phenylindolocarbazolyl)biphenyl; di(terphenylyl)indolocarbazole; phenyl(quarterphenylyl)indolocarbazole; phenyl(terphenylyl)indolocarbazole-d₂; bis(phenylindolocarbazolyl)benzene; (quarterphenylyl)dimethylindenocarbazole; (quarterphenylyl)dimethylindenocarbazole; [(terphenylyl)carbazolyl]dimethylindenocarbazole; (terphenylyl)oxazaindenofluorene; bis(oxazaindenofluorenylbiphenylyl); (quarterphenylyl)oxazaindenofluorene; (quinquephenylyl)benzothienocarbazole; (terphenylyl)benzothienocarbazole; phenyl(terphenylyl)indolophenoxazine; phenyl(terphenylyl)indoloacridone; phenyl(terphenylyl)indolophenothiazine; di(biphenylyl)indolophenoxazine; di(biphenylyl)indolophenothiazine; di(biphenylyl)indoloacridone; phenyl(terphenylyl)indolophenoxazine; phenyl(terphenylyl)indolophenothiazine; phenyl(terphenylyl)indoloacridone; phenyl(terphenylyl)spiroindolo(acridinefluorene); phenyl(quarterphenylyl)indolophenothiazine; phenyl(terphenylyl)indolophenoxazine; phenyl(terphenylyl)indoloacridone; phenyl(biphenylyl)spiroindolo(phenothiazinefluorene); phenyl(terphenylyl)benzindolocarbazole

### (Group 26A)

(phenyl)(biphenylyl)[(phenylbicarbazolyl)phenyl]triazine; di(phenyl)[(phenylbicarbazolyl)biphenyl]triazine; di(phenyl)[(biphenylbicarbazolyl)phenyl]triazine; di(phenyl)[(diphenylbicarbazolyl)phenyl]triazine; (phenyl)(cyanotriphenylenyl)bicarbazole; (methylphenyl)(cyanotriphenylenyl)bicarbazole; (tert-butylphenyl)(cyanotriphenylenyl)bicarbazole

### (Group 27A)

CBP(dicarbazolebiphenylyl); PVK(poly(n-vinylcarbazone)); ADN(di(naphthalenyl)anthracene); TCTA; TPBI(tris(phenylbenzimidazolyl)benzene)); Ir(pq)₂(acac); PtOEP; Ir(piq)₃; Btp₂; Ir(acac); Ir(ppy)₃; Ir(ppy)₂(acac); Ir(mpyp)₃; Flrpic; (F2ppy)₂Irtmd); Ir(dfppz)₃; mCP(dicarbazolebenzene); mCBP(di(carbazolyl)biphenylyl); CBP(bis(carbazolyl)biphenylyl); TPBI(tris(phenylbenzimidazolyl)benzene)); BmPyPb(tri[(pyridyl)phenyl]benzene); TSPO1 (diphenylphosphine oxidetriphenylsilylphenyl); Alq3(tris(quinolinolato)aluminum; COT(octa-substituted cyclooctatetraene); TAZ; Bphen(diphenylphenanthroline)); BCP; Balq

### (Group 28A)

tris[(methylindolocarbazolyl)phenyl]amine; tris[(azaoxaindenofluorenyl)phenyl]amine; tris[(thiaazaindenobenzofluorenyl)phenyl]amine; tris[(indenocarbazolyl)phenyl]amine; bis[(methylindolocarbazolyl)phenyl][(azaoxaindenofluorenyl)phenyl]amine; tris[(azaoxaindenobenzofluorenyl)phenyl]amine; tris[(azaselenaindenobenzofluorenyl)phenyl]amine; tris[(thiaazaindenofluorenyl)phenyl]amine; [(methylindolocarbazolyl)phenyl]bis[(azaoxaindenofluorenyl)phenyl]amine; tris[(azaoxaindenonaphthofluorenyl)phenyl]amine; tris[(azaselenaindenobenzofluorenyl)phenyl]amine; tris[(phenylindolocarbazolyl)phenyl]amine; tris[(dimethylindolophenazinyl)phenyl]amine; [(dihydroindoloanthracenyl)phenyl]bis[(indolothioxanthenyl)phenyl]amine; tris[(indolophenoxathiinyl)phenyl]amine; tris[(indoloxanthenyl)phenyl]amine; tris[(methylindolobenzophenoxazinyl)phenyl]amine; tris[(indenoacridinyl)phenyl]amine; tris[(benzofuroacridinyl)phenyl]amine; tris[(benzothiophenothiazinyl)phenyl]amine; tris[(phenylbenzothiophenazinyl)phenyl]amine; tris[(dihydrobenzofurophenanthridinyl)phenyl]amine; tetrakis[(methylindolocarbazolyl)phenyl]silane; tetrakis[(azaoxaindenofluorenyl)phenyl]silane; tetrakis[(azaselenaindenofluorenyl)phenyl]silane; tetrakis[(azaoxaindenofluorenyl)phenyl]silane; tetrakis[(azaoxaindenofluorenyl)phenyl]silane; tetrakis[((azaoxadithiapentacenyl)phenyl]silane; tetrakis[(azaoxaindenofluorenyl)phenyl]germane; tetrakis[(methylindolocarbazolyl)phenyl]germane; tetrakis[(methyldi(azaoxathiapentacenyl)phenyl]germane; bis[(benzofurophenoxazinyl)phenyl]bis[(benzothienophenoxazinyl)phenyl]germane; (phenyl)(benzofurocarbazolyl)[(benzofurocarbazolyl)phenyl]phosphine oxide; (phenyl)(benzothienocarbazolyl)[(benzothienocarbazolyl)pyridyl]phosphine oxide; (pyridyl)(methylindolocarbazolyl)[(methylindolocarbazolyl)phenyl]phosphine oxide; (phenyl)(benzothienocarbazolyl)[(benzothienocarbazolyl)phenyl]phosphine oxide; (phenyl)(benzselenocarbazolyl)[(benzselenocarbazolyl)phenyl]phosphine oxide; (phenyl)(azaoxaindenobenzofluorenyl)[(azaoxaindenobenzofluorenyl)phenyl]phosphine oxide; (phenyl)(dimethylindolophenazinyl)[(dimethylindolophenazinyl)phenyl]phosphine oxide; (phenyl)(indolothioxanthenyl)[(indolothioxanthenyl)pyridyl]phosphine oxide; (phenyl)(methylindolophenothiazinyl)[(methylindolophenothiazinyl)pyridyl]phosphine oxide; bis[(azaoxaindenofluorenyl)phenyl]furan; bis[(methylindolophenothiazinyl)phenyl]furan; bis[(thiaazaindenofluorenyl)phenyl]thiophene; bis[(azaselenaindenofluorenyl)pyridyl]thiophene; bis[(thiaazaindenofluorenyl)phenyl]selenophene; bis[(phenylindoloacridinyl)phenyl]selenophene; dimethylbis[(thiaazaindenofluorenyl)phenyl]cyclopentadiene; dimethylbis[(phenylindolobenzocarbazolyl)pyridyl]cyclopentadiene; bis[(benzothienophenothiazinyl)phenyl]oxazole; bis[(benzofurophenoxazinyl)phenyl]oxadiazole; bis[(benzothienophenoxazinyl)phenyl]thiadiazole

### (Group 29A)

di(phenyl)[(phenylbicarbazolyl)phenyl]triazine; di(phenyl)[(phenylbicarbazolyl)phenyl]triazine; di(phenyl)[(phenylbicarbazolyl)phenyl]triazine; di(phenyl)(biphenylbicarbazolyl)triazine; di(phenyl)(biphenylbicarbazolyl)triazine; di(phenyl)(biphenylbicarbazolyl)triazine; (phenyl)(biphenylyl)(phenylbicarbazolyl)triazine; (phenyl)(biphenylyl)(phenylbicarbazolyl)triazine; (phenyl)(biphenylyl)(phenylbicarbazolyl)triazine; (phenyl)(biphenylyl)(phenylbicarbazolyl)triazine; (phenyl)(biphenylyl)(phenylbicarbazolyl)triazine; (phenyl)(biphenylyl)(phenylbicarbazolyl)triazine; di(phenyl)(biphenylbicarbazolyl)triazine; di(phenyl)(biphenylbicarbazolyl)triazine; di(phenyl)(biphenylbicarbazolyl)triazine; di(phenyl)[(methylphenylbicarbazolyl)phenyl]triazine; di(phenyl)[(methylphenylbicarbazolyl)phenyl]triazine; di(phenyl)[methyl(phenylbicarbazolyl)phenyl]triazine; di(phenyl)[(fluorophenylbicarbazolyl)phenyl]triazine; di(phenyl)[fluoro(phenylbicarbazolyl)phenyl]triazine; (phenyl)(fluorophenyl)[(phenylbicarbazolyl)phenyl]triazine; di(phenyl)[(cyclohexylphenylbicarbazolyl)phenyl]triazine; di(phenyl)[(trimethylsilylphenylbicarbazolyl)phenyl]triazine; phenyl(cyanophenanthryl)bicarbazole

### (Group 30A)

dicarbazolylbenzene; dicarbazolylbiphenyl; bis[(oxodiphenylphosphino)phenyl]ether; bis[(dimethyldihydroacridinyl)phenyl]sulfone; di(carbazolyl)benzophosphine; bis(dimethoxycarbazolyl)benzophosphine

### (Group 31A)

(phenyl)(biphenylyl)(phenylindolocarbazolyl)triazine; (phenyl)(biphenylyl)(phenylindolocarbazolyl)triazine; di(phenyl)(biphenylindolocarbazolyl)triazine; di(phenyl)(biphenylindolocarbazolyl)triazine; (phenyl)(terphenylyl)(phenylindolocarbazolyl)triazine; (phenyl)(terphenylyl)(phenylindolocarbazolyl)triazine; (phenyl)(terphenylyl)(phenylindolocarbazolyl)triazine; (phenyl)(terphenylyl)(phenylindolocarbazolyl)triazine; di(phenyl)(terphenylindolocarbazolyl)triazine; di(phenyl)(terphenylindolocarbazolyl)triazine; di(phenyl)(terphenylindolocarbazolyl)triazine; di(phenyl)(terphenylindolocarbazolyl)triazine; di(biphenylyl)(phenylindolocarbazolyl)triazine; di(biphenylyl)(phenylindolocarbazolyl)triazine; di(biphenylyl)(phenylindolocarbazolyl)triazine; (phenyl)(biphenylyl)(dibutylphenylindolocarbazolyl)triazine; (phenyl)(biphenylyl)[di(tert-butyl)phenylindolocarbazolyl]triazine; (phenyl)(terphenylyl)(dibutylphenylindolocarbazolyl)triazine; (phenyl)(terphenylyl)[di(tert-butyl)phenylindolocarbazolyl]triazine; (phenyl)(biphenylyl)(dibutylphenylindolocarbazolyl)triazine; (phenyl)(biphenylyl)[di(tert-butyl)phenylindolocarbazolyl]triazine; (phenyl)(terphenylyl)[di(tert-butyl)phenylindolocarbazolyl]triazine; (phenyl)(terphenylyl)(dibutylphenylindolocarbazolyl)triazine; (biphenylyl)(biphenylyl)(dibutylphenylindolocarbazolyl)triazine; (phenyl)(biphenylyl)(phenylindolocarbazolyl)triazine; (phenyl)(biphenylyl)(phenylindolocarbazolyl)triazine; di(phenyl)(biphenylindolocarbazolyl)triazine; di(phenyl)(biphenylindolocarbazolyl)triazine; (phenyl)(terphenylyl)(phenylindolocarbazolyl)triazine; (phenyl)(terphenylyl)(phenylindolocarbazolyl)triazine; (phenyl)(terphenylyl)(phenylindolocarbazolyl)triazine; (phenyl)(terphenylyl)(phenylindolocarbazolyl)triazine; di(phenyl)(terphenylindolocarbazolyl)triazine; di(phenyl)(terphenylindolocarbazolyl)triazine; di(phenyl)(terphenylindolocarbazolyl)triazine; di(biphenylyl)(phenylindolocarbazolyl)triazine; di(biphenylyl)(phenylindolocarbazolyl)triazine; di(biphenylyl)(phenylindolocarbazolyl)triazine; di(biphenylyl)(phenylindolocarbazolyl)triazine; (phenyl)(biphenylyl)(dibutylphenylindolocarbazolyl)triazine; (phenyl)(biphenylyl)[di(tert-butyl)phenylindolocarbazolyl]triazine; (phenyl)(terphenylyl)(dibutylphenylindolocarbazolyl)triazine; (phenyl)(terphenylyl)[di(tert-butyl)phenylindolocarbazolyl]triazine; (phenyl)(biphenylyl)(dibutylphenylindolocarbazolyl)triazine; (phenyl)(biphenylyl)[di(tert-butyl)phenylindolocarbazolyl]triazine; (phenyl)(terphenylyl)[di(tert-butyl)phenylindolocarbazolyl]triazine; (phenyl)(terphenylyl)(dibutylphenylindolocarbazolyl)triazine; (biphenylyl)(terphenylyl)(dibutylphenylindolocarbazolyl)triazine; (phenyl)(biphenylyl)(phenylindolocarbazolyl)triazine; (phenyl)(biphenylyl)(phenylindolocarbazolyl)triazine; di(phenyl)(biphenylindolocarbazolyl)triazine; di(phenyl)(biphenylindolocarbazolyl)triazine; (phenyl)(terphenylyl)(phenylindolocarbazolyl)triazine; (phenyl)(terphenylyl)(phenylindolocarbazolyl)triazine; (phenyl)(terphenylyl)(phenylindolocarbazolyl)triazine; (phenyl)(terphenylyl)(phenylindolocarbazolyl)triazine; di(phenyl)(terphenylindolocarbazolyl)triazine; di(phenyl)(terphenylindolocarbazolyl)triazine; di(phenyl)(terphenylindolocarbazolyl)triazine; di(phenyl)(terphenylindolocarbazolyl)triazine; di(biphenylyl)(phenylindolocarbazolyl)triazine; di(biphenylyl)(phenylindolocarbazolyl)triazine; di(biphenylyl)(phenylindolocarbazolyl)triazine; (phenyl)(biphenylyl)(dibutylphenylindolocarbazolyl)triazine; (phenyl)(biphenylyl)[di(tert-butyl)phenylindolocarbazolyl]triazine; (phenyl)(terphenylyl)(dibutylphenylindolocarbazolyl)triazine; (phenyl)(terphenylyl)[di(tert-butyl)phenylindolocarbazolyl]triazine; (phenyl)(biphenylyl)(dibutylphenylindolocarbazolyl)triazine; (phenyl)(biphenylyl)[di(tert-butyl)phenylindolocarbazolyl]triazine; (phenyl)(terphenylyl)(dibutylphenylindolocarbazolyl)triazine; (phenyl)(terphenylyl)[di(tert-butyl)phenylindolocarbazolyl]triazine; (biphenylyl)(terphenylyl)[di(tert-butyl)phenylindolocarbazolyl]triazine; (phenyl) (biphenylyl)(phenylindolocarbazolyl)triazine; (phenyl)(biphenylyl)(phenylindolocarbazolyl)triazine; di(phenyl)(biphenylindolocarbazolyl)triazine; di(phenyl)(biphenylindolocarbazolyl)triazine; (phenyl)(terphenylyl)(phenylindolocarbazolyl)triazine; (phenyl)(terphenylyl)(phenylindolocarbazolyl)triazine; (phenyl)(terphenylyl)(phenylindolocarbazolyl)triazine; (phenyl)(terphenylyl)(phenylindolocarbazolyl)triazine; di(biphenylyl)(phenylindolocarbazolyl)triazine; di(biphenylyl)(phenylindolocarbazolyl)triazine; (phenyl)(biphenylyl)(phenylindolocarbazolyl)triazine; (phenyl)(biphenylyl)(phenylindolocarbazolyl)triazine; (phenyl)(terphenylyl)(phenylindolocarbazolyl)triazine; (phenyl)(terphenylyl)(phenylindolocarbazolyl)triazine; (phenyl)(terphenylyl)(phenylindolocarbazolyl)triazine; (phenyl)(terphenylyl)(phenylindolocarbazolyl)triazine; di(biphenylyl)(phenylindolocarbazolyl)triazine; di(biphenylyl)(phenylindolocarbazolyl)triazine; (phenyl)(biphenylyl)(phenylindolocarbazolyl)triazine; (phenyl)(biphenylyl)(phenylindolocarbazolyl)triazine; di(phenyl)(biphenylindolocarbazolyl)triazine; di(phenyl)(biphenylindolocarbazolyl)triazine; (phenyl)(terphenylyl)(phenylindolocarbazolyl)triazine; (phenyl)(terphenylyl)(phenylindolocarbazolyl)triazine; (phenyl)(terphenylyl)(phenylindolocarbazolyl)triazine; (phenyl)(terphenylyl)(phenylindolocarbazolyl)triazine; di(biphenylyl)(phenylindolocarbazolyl)triazine; di(biphenylyl)(phenylindolocarbazolyl)triazine; di(terphenylyl)(phenylindolocarbazolyl)triazine; di(terphenylyl)(phenylindolocarbazolyl)triazine; di(terphenylyl)(phenylindolocarbazolyl)triazine; di(terphenylyl)(phenylindolocarbazolyl)triazine; (phenyl)(biphenylyl)[phenyl(dibenzofuranyl)indolocarbazolyl]triazine; (phenyl)(biphenylyl)[phenyl(dibenzothiophenyl)indolocarbazolyl]triazine; (phenyl)(terphenylyl)[phenyl(dibenzofuranyl)indolocarbazolyl]triazine; (phenyl)(terphenylyl)[phenyl(dibenzothiophenyl)indolocarbazolyl]triazine; (phenyl)(biphenylyl)(biphenylindolocarbazolyl)triazine; (phenyl)(biphenylyl)(biphenylindolocarbazolyl)triazine; (phenyl)(biphenylyl)(biphenylindolocarbazolyl)triazine; (phenyl)(biphenylyl)(biphenylindolocarbazolyl)triazine; (phenyl)(biphenylyl)(biphenylindolocarbazolyl)triazine; (phenyl)(biphenylyl)(biphenylindolocarbazolyl)triazine; (phenyl)(biphenylyl)(biphenylindolocarbazolyl)triazine; (phenyl)(biphenylyl)(biphenylindolocarbazolyl)triazine; (phenyl)(terphenylyl)(biphenylindolocarbazolyl)triazine; (phenyl)(biphenylyl)bicarbazole; (phenyl)(biphenylyl)bicarbazole; di(biphenylyl)bicarbazole; (phenyl)(biphenylyl)bicarbazole; (phenyl)(biphenylyl)bicarbazole; di(biphenylyl)bicarbazole; (phenyl)(biphenylyl)bicarbazole; (phenyl)(biphenylyl)bicarbazole; (phenyl)(biphenylyl)bicarbazole; (phenyl)(biphenylyl)bicarbazole; di(biphenylyl)bicarbazole; (phenyl)(biphenylyl)bicarbazole; (phenyl)(biphenylyl)bicarbazole; (phenyl)(biphenylyl)bicarbazole; di(biphenylyl)bicarbazole; (phenyl)(biphenylyl)bicarbazole; (phenyl)(biphenylyl)bicarbazole; di(biphenylyl)bicarbazole; (phenyl)(biphenylyl)bicarbazole; (phenyl)(biphenylyl)bicarbazole; (phenyl)(biphenylyl)bicarbazole; (phenyl)(biphenylyl)bicarbazole; di(biphenylyl)bicarbazole; (phenyl)(biphenylyl)bicarbazole; (phenyl)(biphenylyl)bicarbazole; (phenyl)(biphenylyl)bicarbazole; di(biphenylyl)bicarbazole; (phenyl)(biphenylyl)bicarbazole; (phenyl)(biphenylyl)bicarbazole; di(biphenylyl)bicarbazole; (phenyl)(biphenylyl)bicarbazole; (phenyl)(biphenylyl)bicarbazole; (phenyl)(biphenylyl)bicarbazole; (phenyl)(biphenylyl)bicarbazole; (phenyl)(biphenylyl)bicarbazole; di(biphenylyl)bicarbazole; (phenyl)(terphenylyl)bicarbazole; (phenyl)(terphenylyl)bicarbazole; (biphenylyl)(terphenylyl)bicarbazole; (phenyl)(terphenylyl)bicarbazole; (phenyl)(terphenylyl)bicarbazole; (biphenylyl)(terphenylyl)bicarbazole; di(biphenylyl)bicarbazole; di(biphenylyl)bicarbazole

### (Group 32A)

(phenyl)(carbazolylphenyl)(dibenzofuranyl)triazine; (biphenylyl)(carbazolylphenyl)(dibenzofuranyl)triazine; (terphenylyl)(carbazolylphenyl)(dibenzofuranyl)triazine; (biphenylyl)(carbazolylphenyl)(dibenzofuranyl)triazine; (terphenylyl)(carbazolylphenyl)(dibenzofuranyl)triazine; (phenyl)(phenylcarbazolylphenyl)(dibenzofuranyl)triazine; (terphenylyl)(carbazolylphenyl)(dibenzofuranyl)triazine; (terphenylyl)(carbazolylphenyl)(dibenzofuranyl)triazine; (phenyl)(phenylcarbazolylphenyl)(dibenzofuranyl)triazine; (phenyl)(phenylcarbazolylphenyl)(dibenzofuranyl)triazine; (phenyl)(diphenylcarbazolylphenyl)(dibenzofuranyl)triazine; (phenyl)(biphenylcarbazolylphenyl)(dibenzofuranyl)triazine; (phenyl)(biphenylcarbazolylphenyl)(dibenzofuranyl)triazine; (phenyl)(biphenylcarbazolylphenyl)(dibenzofuranyl)triazine; (biphenylyl)(phenylcarbazolylphenyl)(dibenzofuranyl)triazine; (phenyl)(carbazolylbiphenylyl)(dibenzofuranyl)triazine; (phenyl)(phenylcarbazolylbiphenylyl)(dibenzofuranyl)triazine; (phenyl)(carbazolylterphenylyl)(dibenzofuranyl)triazine; (biphenylyl)(carbazolylbiphenylyl)(dibenzofuranyl)triazine; (phenyl)(phenylcarbazolylbiphenylyl)(dibenzofuranyl)triazine; (phenyl)(carbazolylbiphenylyl)(dibenzofuranyl)triazine; (biphenylyl)(carbazolylbiphenylyl)(dibenzofuranyl)triazine; (biphenylyl)(carbazolylbiphenylyl)(dibenzofuranyl)triazine; (terphenylyl)(carbazolylbiphenylyl)(dibenzofuranyl)triazine; (phenyl)(carbazolylterphenylyl)(dibenzofuranyl)triazine; (phenyl)(phenylcarbazolylbiphenylyl)(dibenzofuranyl)triazine; (phenyl)(phenylcarbazolylbiphenylyl)(dibenzofuranyl)triazine; (phenyl)(phenylcarbazolylbiphenylyl)(dibenzofuranyl)triazine; (biphenylyl)(carbazolylbiphenylyl)(dibenzofuranyl)triazine; (terphenylyl)(carbazolylbiphenylyl)(dibenzofuranyl)triazine; (phenyl)(diphenylcarbazolylbiphenylyl)(dibenzofuranyl)triazine; (phenyl)(carbazolylbiphenylyl)(dibenzofuranyl)triazine; (biphenylyl)(carbazolylbiphenylyl)(dibenzofuranyl)triazine; (phenyl)(phenylcarbazolylbiphenylyl)(dibenzofuranyl)triazine; (phenyl)(phenylcarbazolylbiphenylyl)(dibenzofuranyl)triazine; (biphenylyl)(phenylcarbazolylbiphenylyl)(dibenzofuranyl)triazine; (phenyl)(carbazolylterphenylyl)(dibenzofuranyl)triazine; (phenyl)(carbazolylterphenylyl)(dibenzofuranyl)triazine; (carbazolylphenyl)bis(dibenzofuranyl)triazine; (carbazolylbiphenylyl)bis(dibenzofuranyl)triazine; (phenyl)(carbazolylphenyl)(dibenzothiophenyl)triazine; (biphenylyl)(carbazolylphenyl)(dibenzothiophenyl)triazine; (terphenylyl)(carbazolylphenyl)(dibenzothiophenyl)triazine; (biphenylyl)(carbazolylphenyl)(dibenzothiophenyl)triazine; (terphenylyl)(carbazolylphenyl)(dibenzothiophenyl)triazine; (terphenylyl)(carbazolylphenyl)(dibenzothiophenyl)triazine; (terphenylyl)(carbazolylphenyl)(dibenzothiophenyl)triazine; (phenyl)(phenylcarbazolylphenyl)(dibenzothiophenyl)triazine; (phenyl)(phenylcarbazolylphenyl)(dibenzothiophenyl)triazine; (biphenylyl)(phenylcarbazolylphenyl)(dibenzothiophenyl)triazine; (phenyl)(diphenylcarbazolylphenyl)(dibenzothiophenyl)triazine; (phenyl)(biphenylcarbazolylphenyl)(dibenzothiophenyl)triazine; (phenyl)(biphenylcarbazolylphenyl)(dibenzothiophenyl)triazine; (phenyl)(biphenylcarbazolylphenyl)(dibenzothiophenyl)triazine; (biphenylyl)(phenylcarbazolylphenyl)(dibenzothiophenyl)triazine; (phenyl)(carbazolylbiphenylyl)(dibenzothiophenyl)triazine; (biphenylyl)(carbazolylbiphenylyl)(dibenzothiophenyl)triazine; (phenyl)(carbazolylbiphenylyl)(dibenzothiophenyl)triazine; (phenyl)(carbazolylbiphenylyl)(dibenzothiophenyl)triazine; (phenyl)(carbazolylterphenylyl)(dibenzothiophenyl)triazine; (phenyl)(carbazolylbiphenylyl)(dibenzothiophenyl)triazine; (biphenylyl)(carbazolylbiphenylyl)(dibenzothiophenyl)triazine; (biphenylyl)(carbazolylbiphenylyl)(dibenzothiophenyl)triazine; (terphenylyl)(carbazolylbiphenylyl)(dibenzothiophenyl)triazine; (phenyl)(carbazolylterphenylyl)(dibenzothiophenyl)triazine; (phenyl)(phenylcarbazolylbiphenylyl)(dibenzothiophenyl)triazine; (phenyl)(carbazolylbiphenylyl)(dibenzothiophenyl)triazine; (biphenylyl)(carbazolylbiphenylyl)(dibenzothiophenyl)triazine; (terphenylyl)(carbazolylbiphenylyl)(dibenzothiophenyl)triazine; (phenyl)(carbazolylterphenylyl)(dibenzothiophenyl)triazine; (phenyl)(phenylcarbazolylbiphenylyl)(dibenzothiophenyl)triazine; (phenyl)(phenylcarbazolylbiphenylyl)(dibenzothiophenyl)triazine; (phenyl)(phenylcarbazolylbiphenylyl)(dibenzothiophenyl)triazine; (phenyl)(diphenylcarbazolylbiphenylyl)(dibenzothiophenyl)triazine; (phenyl)(carbazolylbiphenylyl)(dibenzothiophenyl)triazine; (biphenylyl)(carbazolylbiphenylyl)(dibenzothiophenyl)triazine; (terphenylyl)(carbazolylbiphenylyl)(dibenzothiophenyl)triazine; (biphenylyl)(carbazolylbiphenylyl)(dibenzothiophenyl)triazine; (phenyl)(phenylcarbazolylbiphenylyl)(dibenzothiophenyl)triazine; (phenyl)(phenylcarbazolylbiphenylyl)(dibenzothiophenyl)triazine; (biphenylyl)(phenylcarbazolylbiphenylyl)(dibenzothiophenyl)triazine; (phenyl)(carbazolylterphenylyl)(dibenzothiophenyl)triazine; (phenyl)(carbazolylterphenylyl)(dibenzothiophenyl)triazine; (carbazolylphenyl)bis(dibenzothiophenyl)triazine; (carbazolylbiphenylyl)bis(dibenzothiophenyl)triazine; (diphenylpyridyl)(phenyl)bicarbazole; (diphenylpyridyl)(phenyl)bicarbazole; (diphenylpyridyl)(biphenylyl)bicarbazole; (diphenylpyridyl)(terphenylyl)bicarbazole; (terphenylyl)(phenyl)bicarbazole; (diphenylpyridyl)(naphthyl)bicarbazole; (diphenylpyridyl)(naphthyl)bicarbazole; (diphenylpyridyl)(triphenylenyl)bicarbazole; (diphenylpyridyl)(triphenylenyl)bicarbazole; bis(diphenylpyridyl)bicarbazole; bis(diphenylpyridyl)bicarbazole; bis(diphenylpyridyl)bicarbazole; (dimethylfluorenyl)(phenyl)bicarbazole; (dimethylfluorenyl)(biphenylyl)bicarbazole; (dimethylfluorenyl)(terphenylyl)bicarbazole; (dibenzofuranyl)(phenyl)bicarbazole; (dibenzofuranyl)(biphenylyl)bicarbazole; (dibenzofuranyl)(terphenylyl)bicarbazole; (dibenzothiophenyl)(phenyl)bicarbazole; (dibenzothiophenyl)(biphenylyl)bicarbazole; (dibenzothiophenyl)(terphenylyl)bicarbazole; diphenylbicarbazole; dipyridylbicarbazole; dipyrimidylbicarbazole; (triphenylenyl)(phenyl)bicarbazole; (pyridyl)(triphenylenyl)bicarbazole; (phenyl)(triphenylenyl)bicarbazole; (pyridyl)(triphenylenyl)bicarbazole; (phenyl)(triphenylenyl)bicarbazole; (pyridyl)(triphenylenyl)bicarbazole; di(biphenylyl)bicarbazole; (biphenylyl)(terphenylyl)bicarbazole; (biphenylyl)(terphenylyl)bicarbazole; (dimethylfluorenyl)(phenyl)bicarbazole; (dimethylfluorenyl)(biphenylyl)bicarbazole; (dimethylfluorenyl)(biphenylyl)bicarbazole; (dimethylfluorenyl)(terphenylyl)bicarbazole; (dibenzofuranyl)(biphenylyl)bicarbazole; (dibenzothiophenyl)(biphenylyl)bicarbazole; diphenyltercarbazole; (dibenzofuranyl)(phenyl)bicarbazole; (dibenzofuranyl)(biphenylyl)bicarbazole; (dibenzofuranyl)(terphenylyl)bicarbazole; (dibenzofuranyl)(biphenylyl)bicarbazole; (dibenzothiophenyl)(phenyl)bicarbazole; (dibenzothiophenyl)(biphenylyl)bicarbazole; (dibenzothiophenyl)(terphenylyl)bicarbazole; (dibenzothiophenyl)(biphenylyl)bicarbazole; (dimethylfluorenyl)(diphenylpyridyl)bicarbazole; (dimethylfluorenyl)(diphenylpyridyl)bicarbazole; (dimethylfluorenyl)(pyridylbiphenylyl)bicarbazole; (dimethylfluorenyl)(pyridylbiphenylyl)bicarbazole; (dimethylfluorenyl)(pyridylbiphenylyl)bicarbazole; (dibenzofuranyl)(diphenylpyridyl)bicarbazole; (dibenzofuranyl)(pyridylbiphenylyl)bicarbazole; (dibenzofuranyl)(pyridylbiphenylyl)bicarbazole; (dibenzofuranyl)(pyridylbiphenylyl)bicarbazole; (dibenzothiophenyl)(diphenylpyridyl)bicarbazole; (dibenzothiophenyl)(pyridylbiphenylyl)bicarbazole; (dibenzothiophenyl)(pyridylbiphenylyl)bicarbazole; (dibenzothiophenyl)(pyridylbiphenylyl)bicarbazole; (diphenylpyridyl)(phenyl)bicarbazole; (pyridylbiphenylyl)(phenyl)bicarbazole; (diphenylpyridyl)(phenyl)bicarbazole; (diphenylpyridyl)(biphenylyl)bicarbazole; (pyridylbiphenylyl)(biphenylyl)bicarbazole; (diphenylpyridyl)(biphenylyl)bicarbazole; (diphenylpyridyl)(biphenylyl)bicarbazole; (diphenylpyridyl)(biphenylyl)bicarbazole; (pyridylbiphenylyl)(biphenylyl)bicarbazole; (diphenylpyridyl)(phenyl)bicarbazole; (diphenylpyridyl)(phenyl)bicarbazole; (pyridylbiphenylyl)(phenyl)bicarbazole; (diphenylpyridyl)(biphenylyl)bicarbazole; (diphenylpyridyl)(biphenylyl)bicarbazole; (pyridylbiphenylyl)(biphenylyl)bicarbazole; (diphenylpyridyl)(biphenylyl)bicarbazole; (diphenylpyridyl)(biphenylyl)bicarbazole; (pyridylbiphenylyl)(biphenylyl)bicarbazole; (dibenzofuranyl)(terphenylyl)bicarbazole; (dibenzofuranyl)(biphenylyl)bicarbazole; (dibenzofuranyl)(phenyl)bicarbazole; (dibenzofuranyl)(biphenylyl)bicarbazole; (dibenzothiophenyl)(terphenylyl)bicarbazole; (dibenzothiophenyl)(biphenylyl)bicarbazole; (dibenzothiophenyl)(phenyl)bicarbazole; (dibenzothiophenyl)(biphenylyl)bicarbazole; (diphenylpyridyl)(biphenylyl)bicarbazole; (diphenylpyridyl)(biphenylyl)bicarbazole; (diphenylpyridyl)(phenyl)bicarbazole; (diphenylpyridyl)(phenyl)bicarbazole; (diphenylpyridyl)(phenyl)bicarbazole; (diphenylpyridyl)(biphenylyl)bicarbazole; (diphenylpyridyl)(phenyl)bicarbazole; (diphenylpyridyl)(phenyl)bicarbazole; (diphenylpyridyl)(phenyl)bicarbazole; (diphenylpyridyl)(phenyl)bicarbazole; (diphenylpyridyl)(phenyl)bicarbazole; (diphenylpyridyl)(phenyl)bicarbazole; (diphenylpyridyl)(phenyl)bicarbazole; (diphenylpyridyl)(phenyl)bicarbazole; (diphenylpyridyl)(biphenylyl)bicarbazole; (diphenylpyridyl)(phenyl)bicarbazole; di(biphenylyl)bicarbazole; di(biphenylyl)bicarbazole; (biphenylyl)(phenyl)bicarbazole; bis(phenylcarbazolyl)benzene; (phenylcarbazolyl)(biphenylcarbazolyl)benzene; bis(biphenylcarbazolyl)benzene; bis(phenylcarbazolyl)benzene; (phenylcarbazolyl)(terphenylcarbazolyl)benzene; bis(biphenylcarbazolyl)benzene; (phenylcarbazolyl)(dimethylfluorenylcarbazolyl)benzene; (dibenzofuranyl)(phenylcarbazolyl)benzene; (dibenzothiophenyl)(phenylcarbazolyl)benzene; bis(phenylcarbazolyl)biphenyl; (phenylcarbazolyl)(biphenylcarbazolyl)benzene; (biphenylcarbazolyl)(terphenylcarbazolyl)benzene; bis(phenylcarbazolyl)benzene; (phenylcarbazolyl)(terphenylcarbazolyl)benzene; bis(biphenylcarbazolyl)benzene; (phenylcarbazolyl)(dimethylfluorenylcarbazolyl)benzene; (phenylcarbazolyl)(terphenylcarbazolyl)benzene; (dibenzothiophenyl)(phenylcarbazolyl)benzene; (biphenylyl)(terphenylyl)bicarbazole; (biphenylyl)(quarterphenylyl)bicarbazole; (terphenylyl)(quarterphenylyl)bicarbazole; (biphenylyl)(terphenylyl)bicarbazole; (terphenylyl)(quarterphenylyl)bicarbazole; di(quarterphenylyl)bicarbazole; (biphenylyl)(quarterphenylyl)bicarbazole; (phenyl)(quarterphenylyl)bicarbazole; (phenyl)(terphenylyl)bicarbazole; di(phenyl)(biphenylyl)bicarbazole; di(phenyl)(biphenylyl)bicarbazole; tri(phenyl)bicarbazole; tri(phenyl)bicarbazole; di(phenyl)(biphenylyl)bicarbazole; di(phenyl)(biphenylyl)bicarbazole; di(phenyl)(biphenylyl)bicarbazole; (phenyl)(biphenylyl)bicarbazole; (dibenzofuranylphenyl)(phenyl)bicarbazole; (dibenzothiophenylphenyl)(phenyl)bicarbazole; diphenylindolocarbazole; (biphenylyl)(phenyl)indolocarbazole; (biphenylyl)(phenyl)indolocarbazole; di(biphenylyl)indolocarbazole; di(biphenylyl)indolocarbazole; (biphenylyl)(terphenylyl)indolocarbazole; (biphenylyl)(terphenylyl)indolocarbazole; di(terphenylyl)indolocarbazole; (terphenylyl)(phenyl)indolocarbazole; (biphenylyl)(terphenylyl)indolocarbazole; (biphenylyl)(terphenylyl)indolocarbazole; di(terphenylyl)indolocarbazole; di(biphenylyl)(phenyl)indolocarbazole; di(phenyl)(biphenylyl)indolocarbazole; di(biphenylyl)(phenyl)indolocarbazole; di(biphenylyl)(phenyl)indolocarbazole; di(biphenylyl)(phenyl)indolocarbazole; di(phenyl)(biphenylyl)indolocarbazole; di(biphenylyl)(phenyl)indolocarbazole; di(biphenylyl)(phenyl)indolocarbazole; di(naphthyl)indolocarbazole; di(naphthyl)indolocarbazole; (naphthyl)(biphenylyl)indolocarbazole; (naphthyl)(biphenylyl)indolocarbazole; (naphthyl)(terphenylyl)indolocarbazole; (naphthyl)(terphenylyl)indolocarbazole; (naphthyl)(terphenylyl)indolocarbazole; (naphthyl)(terphenylyl)indolocarbazole; (naphthylphenyl)(phenyl)indolocarbazole; (naphthylphenyl)(phenyl)indolocarbazole; (naphthylphenyl)(phenyl)indolocarbazole; (naphthylphenyl)(phenyl)indolocarbazole; (naphthylphenyl)(biphenylyl)indolocarbazole; (naphthylphenyl)(biphenylyl)indolocarbazole; (naphthylphenyl)(biphenylyl)indolocarbazole; (naphthylphenyl)(biphenylyl)indolocarbazole; (phenylnaphthyl)(biphenylyl)indolocarbazole; (phenylnaphthyl)(biphenylyl)indolocarbazole; (phenylnaphthyl)(biphenylyl)indolocarbazole; (phenylnaphthyl)(biphenylyl)indolocarbazole; di(phenyl)(phenylnaphthyl)indolocarbazole; di(phenyl)(phenylnaphthyl)indolocarbazole; di(phenyl)(phenylnaphthyl)indolocarbazole; di(phenyl)(phenylnaphthyl)indolocarbazole; di(phenyl)(naphthylphenyl)indolocarbazole; di(phenyl)(naphthylphenyl)indolocarbazole; di(phenyl)(naphthylphenyl)indolocarbazole; di(phenyl)(naphthylphenyl)indolocarbazole; (phenyl)(naphthyl)(naphthylphenyl)indolocarbazole; (phenyl)(biphenylyl)(naphthylphenyl)indolocarbazole; (phenyl)(naphthyl)(naphthylphenyl)indolocarbazole; (phenyl)(biphenylyl)(naphthylphenyl)indolocarbazole

### (Group 33A)

[bis(ditrifluoromethylphenyl)indenofluorenediylidene]bis(malononitrile); (diindenothiophenediylidene)bis(malononitrile); [bis(trifluoromethoxyphenyl)indenofluorenediylidene]bis(malononitrile); [bis(trifluoromethoxyphenyl)indenofluorenediylidene]bis(malononitrile); [bis[fluoro(trifluoromethyl)phenyl]diazaindenofluorenediylidene]bis(malononitrile); [bis(trifluoromethylphenyl)diazaindenofluorenediylidene]bis(malononitrile); [bis(trifluoromethylphenyl)diazaindenofluorenediylidene]bis(aminonitrile); [bis[fluoro(trifluoromethyl)phenyl]diazaindenofluorenediylidene]bis(malononitrile); [bis[fluoro(trifluoromethyl)phenyl]diazaindenofluorenediylidene]bis(aminonitrile); [bis(pentafluorophenyl)diazaindenofluorenediylidene]bis(malononitrile); [bis(pentafluorophenyl)diazaindenofluorenediylidene]bis(aminonitrile); [bis[fluoro(trifluoromethyl)phenyl]diazaindenofluorenediylidene]bis(malononitrile); [bis[fluoro(trifluoromethyl)phenyl]diazaindenofluorenediylidene]bis(aminonitrile); [bis[bis(trifluoromethyl)phenyl]diazaindenofluorenediylidene]bis(malononitrile); [bis[bis(trifluoromethyl)phenyl]diazaindenofluorenediylidene]bis(aminonitrile); [bis(trifluorophenyl)diazaindenofluorenediylidene]bis(malononitrile); [bis(trifluorophenyl)diazaindenofluorenediylidene]bis(aminonitrile); [bis[(trifluoromethoxy)phenyl]diazaindenofluorenediylidene]bis(malononitrile); [bis[(trifluoromethoxy)phenyl]diazaindenofluorenediylidene]bis(aminonitrile); [bis[(trifluoroacetyl)phenyl]diazaindenofluorenediylidene]bis(malononitrile); [bis[(trifluoroacetyl)phenyl]diazaindenofluorenediylidene]bis(aminonitrile); [bis[cyano(trifluoromethyl)phenyl]diazaindenofluorenediylidene]bis(malononitrile); [bis[cyano(trifluoromethyl)phenyl]diazaindenofluorenediylidene]bis(aminonitrile); [bis(trifluoromethyl)diazaindenofluorenediylidene]bis(malononitrile); [bis(trifluoromethyl)diazaindenofluorenediylidene]bis(aminonitrile); (difluorodiazaindenofluorenediylidene)bis(malononitrile); (difluorodiazaindenofluorenediylidene)bis(aminonitrile); (hexafluorodiazaindenofluorenediylidene)bis(malononitrile); (hexafluorodiazaindenofluorenediylidene)bis(aminonitrile); (tetrafluorodiazaindenofluorenediylidene)bis(malononitrile); (tetrafluorodiazaindenofluorenediylidene)bis(aminonitrile); [bis(trifluoromethyl)azaindenofluorenediylidene]bis(malononitrile); (diphenyldiazaindenofluorenediylidene)bis(malononitrile); (difluorodiazaindenofluorenediylidene)bis[(trifluoromethyl)ethyl acrylate]; (difluorodiazaindenofluorenediylidene)bis(methyleneindandione); [bis[fluoro(trifluoromethyl)phenyl]diazaindenofluorenediylidene]bis(malononitrile); [bis[fluoro(trifluoromethyl)phenyl]diazaindenofluorenediylidene]bis(aminonitrile); [bis[bis(trifluoromethyl)phenyl]diazaindenofluorenediylidene]bis(malononitrile); [bis[bis(trifluoromethyl)phenyl]diazaindenofluorenediylidene]bis(aminonitrile); [bis[fluoro(trifluoromethyl)phenyl]diazaindenofluorenediylidene]bis(malononitrile); [bis[fluoro(trifluoromethyl)phenyl]diazaindenofluorenediylidene]bis(aminonitrile); [bis[(trifluoromethyl)phenyl]diazaindenofluorenediylidene]bis(malononitrile); [bis[(trifluoromethyl)phenyl]diazaindenofluorenediylidene]bis(aminonitrile); [bis[(trifluoromethoxy)phenyl]diazaindenofluorenediylidene]bis(malononitrile); [bis[(trifluoromethoxy)phenyl]diazaindenofluorenediylidene]bis(aminonitrile); [bis[cyano(fluoro)phenyl]diazaindenofluorenediylidene]bis(malononitrile); [bis[cyano(fluoro)phenyl]diazaindenofluorenediylidene]bis(aminonitrile); [bis(fluoropyridyl)diazaindenofluorenediylidene]bis(malononitrile); [bis(fluoropyridyl)diazaindenofluorenediylidene]bis(aminonitrile); [bis(difluoropyridyl)diazaindenofluorenediylidene]bis(malononitrile); [bis(difluoropyridyl)diazaindenofluorenediylidene]bis(aminonitrile); [bis[(trifluoromethyl)thiophenyl]diazaindenofluorenediylidene]bis(malononitrile); [bis[(trifluoromethyl)thiophenyl]diazaindenofluorenediylidene]bis(aminonitrile); (dinaphthyldiazaindenofluorenediylidene)bis(malononitrile); (dinaphthyldiazaindenofluorenediylidene)bis(aminonitrile); [bis(heptafluoronaphthyl)diazaindenofluorenediylidene]bis(malononitrile); [bis(heptafluoronaphthyl)diazaindenofluorenediylidene]bis(aminonitrile); [bis[(trifluoromethyl)phenyl]diazaindenofluorenediylidene]bis(malononitrile); [bis[(trifluoromethyl)phenyl]diazaindenofluorenediylidene]bis(aminonitrile); [tetrakis[(trifluoromethyl)phenyl]diazaindenofluorenediylidene]bis(malononitrile); [tetrakis[(trifluoromethyl)phenyl]diazaindenofluorenediylidene]bis(aminonitrile); [bis[(trifluoromethyl)phenyl]diazaindenofluorenediylidene]bis(malononitrile); [bis[(trifluoromethyl)phenyl]diazaindenofluorenediylidene]bis(aminonitrile); [bis[(trifluoromethyl)phenyl]diazaindenofluorenediylidene]bis(malononitrile); [bis[(trifluoromethyl)phenyl]diazaindenofluorenediylidene]bis(aminonitrile); [bis[(trifluoromethyl)phenyl]diazaindenofluorenediylidene]bis(malononitrile); [bis[(trifluoromethyl)phenyl]indenofluorenediylidene]bis(aminonitrile); [bis[(trifluoromethyl)phenyl]indenofluorenediylidene]bis(malononitrile); [bis[bis(trifluoromethyl)phenyl]indenofluorenediylidene]bis(aminonitrile); [bis[bis(trifluoromethyl)phenyl]indenofluorenediylidene]bis(malononitrile); [bis(trifluorophenyl)indenofluorenediylidene]bis(aminonitrile); [bis(trifluorophenyl)indenofluorenediylidene]bis(malononitrile); [bis(pentafluorophenyl)indenofluorenediylidene]bis(aminonitrile); [bis(pentafluorophenyl)indenofluorenediylidene]bis(malononitrile); [bis(cyanophenyl)indenofluorenediylidene]bis(aminonitrile); [bis(cyanophenyl)indenofluorenediylidene]bis(malononitrile); (diphenylindenofluorenediylidene)bis(aminonitrile); (diphenylindenofluorenediylidene]bis(malononitrile); (dinaphthylindenofluorenediylidene)bis(aminonitrile); (dinaphthylindenofluorenediylidene]bis(malononitrile); (difluoroindenofluorenediylidene)bis(aminonitrile); (difluoroindenofluorenediylidene]bis(malononitrile); (hexafluoroindenofluorenediylidene)bis(aminonitrile); (hexafluoroindenofluorenediylidene]bis(malononitrile); (tetrafluoroindenofluorenediylidene)bis(aminonitrile); (tetrafluoroindenofluorenediylidene]bis(malononitrile); [tetrakis(trifluoromethyl)indenofluorenediylidene]bis(aminonitrile); [tetrakis(trifluoromethyl)indenofluorenediylidene]bis(malononitrile); (phenylindenofluorenediylidene)bis(aminonitrile); [phenyl(trifluoromethyl)indenofluorenediylidene]bis(aminonitrile); (diphenylindenofluorenediylidene)bis(aminonitrile); [diphenylbis(trifluoromethyl)indenofluorenediylidene]bis(aminonitrile); [diphenyl(difluoro)indenofluorenediylidene]bis(aminonitrile); [bis(trifluoromethylphenyl)indenofluorenediylidene]bis(aminonitrile); [dimethylbis(trifluoromethyl)indenofluorenediylidene]bis(malononitrile); [bis[(trifluoromethyl)phenyl]indenofluorenediylidene]bis(malononitrile); [bis[(trifluoromethyl)phenyl]indenofluorenediylidene]bis(aminonitrile); [bis(fluoropyridyl)indenofluorenediylidene]bis(malononitrile); [bis(fluoropyridyl)indenofluorenediylidene]bis(aminonitrile); [bis(difluoropyridyl)indenofluorenediylidene]bis(malononitrile); [bis(difluoropyridyl)indenofluorenediylidene]bis(aminonitrile); [bis(cyanofluorophenyl)indenofluorenediylidene]bis(malononitrile); [bis(cyanofluorophenyl)indenofluorenediylidene]bis(aminonitrile); [bis(cyanophenyl)indenofluorenediylidene]bis(malononitrile); [bis(cyanophenyl)indenofluorenediylidene]bis(aminonitrile); [[(trifluoromethyl)phenyl]indenofluorenediylidene]bis(malononitrile); [[(trifluoromethyl)phenyl]indenofluorenediylidene]bis(aminonitrile); [fluoro[(trifluoromethyl)phenyl]indenofluorenediylidene]bis(malononitrile); [fluoro[(trifluoromethyl)phenyl]indenofluorenediylidene]bis(aminonitrile); [(cyanophenyl)indenofluorenediylidene]bis(malononitrile); [(cyanophenyl)indenofluorenediylidene]bis(aminonitrile); [fluoro(cyanophenyl)indenofluorenediylidene]bis(malononitrile); [fluoro(cyanophenyl)indenofluorenediylidene]bis(aminonitrile); [tetrafluoro[(trifluoromethyl)phenyl]indenofluorenediylidene]bis(malononitrile); [tetrafluoro[(trifluoromethyl)phenyl]indenofluorenediylidene]bis(aminonitrile); [fluoro(trifluoromethyl)phenyl]indenofluorenediylidene]bis(malononitrile); [[fluoro(trifluoromethyl)phenyl]indenofluorenediylidene]bis(aminonitrile); [bis(trifluoromethyl)phenyl]indenofluorenediylidene]bis(malononitrile); [bis(trifluoromethyl)phenyl]indenofluorenediylidene]bis(aminonitrile); [biphenyl(trifluoromethyl)indenofluorenediylidene]bis(malononitrile); [biphenyl(trifluoromethyl)indenofluorenediylidene]bis(aminonitrile); [naphthyl(trifluoromethyl)indenofluorenediylidene]bis(malononitrile); [naphthyl(trifluoromethyl)indenofluorenediylidene]bis(aminonitrile); [bis(fluorophenyl)indenofluorenediylidene]bis(malononitrile); [bis(fluorophenyl)indenofluorenediylidene]bis(aminonitrile); [bis(fluorophenyl)indenofluorenediylidene]bis(malononitrile); [bis(fluorophenyl)indenofluorenediylidene]bis(aminonitrile); [bis(difluorophenyl)indenofluorenediylidene]bis(malononitrile); [bis(difluorophenyl)indenofluorenediylidene]bis(aminonitrile); [bis(difluorophenyl)indenofluorenediylidene]bis(malononitrile); [bis(difluorophenyl)indenofluorenediylidene]bis(aminonitrile); [bis[fluoro(trifluoromethyl)phenyl]indenofluorenediylidene]bis(malononitrile); [bis[fluoro(trifluoromethyl)phenyl]indenofluorenediylidene]bis(aminonitrile); [bis[fluoro(trifluoromethyl)phenyl]indenofluorenediylidene]bis(malononitrile); [bis[fluoro(trifluoromethyl)phenyl]indenofluorenediylidene]bis(aminonitrile); [bis[(trifluoromethyl)phenyl]indenofluorenediylidene]bis(malononitrile); [bis[(trifluoromethyl)phenyl]indenofluorenediylidene]bis(aminonitrile); [bis[fluoro(trifluoromethyl)phenyl]indenofluorenediylidene]bis(malononitrile); [bis[fluoro(trifluoromethyl)phenyl]indenofluorenediylidene]bis(aminonitrile); [bis[bis(trifluoromethyl)phenyl]indenofluorenediylidene]bis(malononitrile); [bis[bis(trifluoromethyl)phenyl]indenofluorenediylidene]bis(aminonitrile); [bis[fluoro(trifluoromethyl)phenyl]indenofluorenediylidene]bis(malononitrile); [bis[fluoro(trifluoromethyl)phenyl]indenofluorenediylidene]bis(aminonitrile); [bis(trifluorophenyl)indenofluorenediylidene]bis(malononitrile); [bis(trifluorophenyl)indenofluorenediylidene]bis(aminonitrile); [bis(fluorophenyl)indenofluorenediylidene]bis(malononitrile); [bis(fluorophenyl)indenofluorenediylidene]bis(aminonitrile); [bis[(trifluoromethyl)phenyl]indenofluorenediylidene]bis(malononitrile); [bis[(trifluoromethyl)phenyl]indenofluorenediylidene]bis(aminonitrile); [bis[fluoro(trifluoromethyl)phenyl]indenofluorenediylidene]bis(malononitrile); [bis[fluoro(trifluoromethyl)phenyl]indenofluorenediylidene]bis(aminonitrile); [bis[difluoro(trifluoromethyl)phenyl]indenofluorenediylidene]bis(malononitrile); [bis[difluoro(trifluoromethyl)phenyl]indenofluorenediylidene]bis(aminonitrile); [bis[difluoro(trifluoromethyl)phenyl]indenofluorenediylidene]bis(malononitrile); [bis[difluoro(trifluoromethyl)phenyl]indenofluorenediylidene]bis(aminonitrile); (dinaphthylindenofluorenediylidene)bis(malononitrile); (dinaphthylindenofluorenediylidene)bis(aminonitrile); (difluoroindenofluorenediylidene)bis(malononitrile); (difluoroindenofluorenediylidene)bis(aminonitrile); [bis(trifluoromethyl)indenofluorenediylidene]bis(malononitrile); [bis(trifluoromethyl)indenofluorenediylidene]bis(aminonitrile); (dicyanoindenofluorenediylidene)bis(malononitrile); (dicyanoindenofluorenediylidene)bis(aminonitrile); (hexafluoroindenofluorenediylidene)bis(malononitrile); (hexafluoroindenofluorenediylidene)bis(aminonitrile); [dicyanobis(trifluoromethyl)indenofluorenediylidene]bis(malononitrile); [dicyanobis(trifluoromethyl)indenofluorenediylidene]bis(aminonitrile); [tetrakis[(trifluoromethyl)phenyl]indenofluorenediylidene]bis(malononitrile); [tetrakis[(trifluoromethyl)phenyl]indenofluorenediylidene]bis(malononitrile); [tetrakis[(trifluoromethyl)phenyl]indenofluorenediylidene]bis(malononitrile); [bis[bis(trifluoromethyl)phenyl]azaindenofluorenediylidene]bis(malononitrile); [bis[bis(trifluoromethyl)phenyl]diazaindenofluorenediylidene]bis(malononitrile); [bis[fluoro(trifluoromethyl)phenyl]diazaindenofluorenediylidene]bis(malononitrile); [bis[fluoro(trifluoromethyl)phenyl]diazaindenofluorenediylidene]bis(malononitrile); [bis[(trifluoromethyl)phenyl]indenofluorenediylidene]bis(trifluoromethylacrylonitrile); [bis(trifluoromethoxy)indenofluorenediylidene]bis(malononitrile); [fluorobis(trifluoromethoxy)indenofluorenediylidene]bis(malononitrile); [bis[(trifluoromethoxy)phenyl]indenofluorenediylidene]bis(malononitrile); [bis[fluoro(trifluoromethoxy)phenyl]indenofluorenediylidene]bis(malononitrile); [(trifluoroethoxy)bis[(trifluoromethoxy)phenyl]indenofluorenediylidene]bis(malononitrile); [bis[(pentafluoroethoxy)phenyl]indenofluorenediylidene]bis(malononitrile); [bis[(pentafluoropropoxy)phenyl]indenofluorenediylidene]bis(malononitrile); [bis[(hexafluoroisopropoxy)phenyl]indenofluorenediylidene]bis(malononitrile); [(pentafluoropropoxy)bis(trifluoromethoxy)indenofluorenediylidene]bis(malononitrile); [(trifluoromethoxy)[fluoro(trifluoromethyl)phenyl]indenofluorenediylidene]bis(malononitrile); [(trifluoromethoxy)[(trifluoromethoxy)phenyl]indenofluorenediylidene]bis(malononitrile); [fluoro[(trifluoromethoxy)phenyl]indenofluorenediylidene]bis(malononitrile); (dibenzindenofluorenediylidene)bis(malononitrile); (azadibenzindenofluorenediylidene)bis(malononitrile); (diazadibenzindenofluorenediylidene)bis(malononitrile); [(trifluoromethoxy)benzindenofluorenediylidene]bis(malononitrile); [(trifluoromethoxy)phenyl]bis(trifluoromethoxy)indenofluorenediylidene]bis(malononitrile); [dithiophenylindenofluorenediylidene]bis(malononitrile); [bis(trifluoromethoxy)diazaindenofluorenediylidene]bis(malononitrile); [bis(trifluoromethoxy)hexaazaindenofluorenediylidene]bis(malononitrile); [tetrakis(trifluoromethoxy)indenofluorenediylidene]bis(malononitrile); [tetrakis(trifluoromethoxy)indenofluorenediylidene]bis(malononitrile); hexacyanohexaazatriphenylene; tris[cyano(cyanotetrafluorophenyl)methylene]cyclopropane; di(biphenylyl)(phenylspiroacridinefluorenyl)amine; (biphenylyl)(dimethylfluorenyl)(spiroindoloacridinefluorenyl)amine; (biphenylyl)(dibenzofuranyl)(phenylspiroacridinefluorenyl)amine; di(biphenylyl)(spirobenzanthracenefluorenyl)amine; (biphenylyl)[(dibenzothiophenyl)phenyl](spirobenzanthracenefluorenyl)amine; di(biphenylyl)(dibenzofuranyl)amine; (biphenylyl)(terphenylyl)(dibenzofuranyl)amine; (phenyl)(terphenylyl)(dibenzofuranyl)amine; (biphenylyl)(dibenzofuranyl)[(carbazolyl)phenyl]amine; (terphenylyl)(naphthyl)(dibenzofuranyl)amine; (phenyl)(dibenzofuranyl)[(dibenzofuranyl)phenyl]amine; (terphenylyl)(biphenylyl)(dibenzofuranyl)amine; (biphenylyl)(dibenzofuranyl)(dimethylfluorenyl)amine; (naphthyl)(biphenylyl)(dibenzofuranyl)amine; di(biphenylyl)[(phenyldibenzofuranyl)phenyl]amine; (naphthyl)(naphthylphenyl)[(phenyldibenzofuranyl)phenyl]amine; (naphthyl)(naphthylphenyl)[(phenyldibenzofuranyl)phenyl]amine; (biphenylyl)[(dimethylfluorenyl)phenyl][(dibenzofuranyl)phenyl]amine; (naphthyl)(biphenylyl)[(phenyldibenzofuranyl)biphenyl]amine; (naphthyl)(biphenylyl)[(dibenzofuranyl)phenyl]amine; (phenyl)(naphthylphenyl)[(phenyldibenzofuranyl)phenyl]amine; (naphthylphenyl)bis[(dibenzofuranyl)phenyl]amine; di(terphenylyl)[(dibenzofuranyl)phenyl]amine; (phenyl)(biphenylyl)[(dibenzofuranyl)dimethylfluorenyl]amine; (phenyl)[(triphenylsilyl)phenyl](diphenylfluorenyl)amine; (phenyl)[(triphenylsilyl)phenyl](triphenylfluorenyl)amine; (diphenylfluorenyl)di(biphenylyl)amine; (diphenylfluorenyl)(biphenylyl)(naphthylphenyl)amine; (diphenylfluorenyl)(biphenylyl)(terphenylyl)amine; (diphenylfluorenyl)(biphenylyl)(phenyldibenzofuranyl)amine; (phenyl)[(triphenylsilyl)phenyl](naphthylphenylfluorenyl)amine; (diphenylfluorenyl)(biphenylyl)(dibenzofuranyl)amine; (triphenylsilyldibenzothiophenyl)(phenyl)(diphenylfluorenyl)amine; (diphenylnaphthylsilylbiphenylyl)(phenyl)(diphenyl)amine; (triphenylsilylphenyl)(phenyl)(naphthylphenylfluorenyl)amine; (diphenylfluorenyl)(biphenylyl)(phenyldibenzothiophenyl)amine; (diphenylfluorenyl)(biphenylyl)(dibenzothiophenyl)amine; (diphenylfluorenyl)(biphenylyl)(dibenzofuranylphenyl)amine; (diphenylfluorenyl)(biphenylyl)(phenyldibenzofuranylphenyl)amine; (diphenylfluorenyl)(biphenylyl)(phenyldibenzothiophenyl)amine; (dimethylfluorenyl)di(biphenylyl)amine; (dimethylfluorenyl)(terphenylyl)(biphenylyl)amine; (dimethylfluorenyl)(biphenylyl)(dibenzofuranylphenyl)amine; (dimethylfluorenyl)(biphenylyl)(dibenzothiophenylphenyl)amine; (dimethylfluorenyl)(biphenylyl)(phenyldibenzofuranyl)amine; (diphenylfluorenyl)bis(methylbiphenylyl)amine; (dimethylfluorenyl)di(biphenylyl)amine; (dimethylfluorenyl)di(biphenylyl)amine; (dibenzofuranylphenyl)(biphenylyl)(dimethylfluorenyl)amine; (quarterphenylyl)(dibenzofuranylphenyl)(phenylcarbazolylphenyl)amine; (quarterphenylyl)(dibenzofuranylphenyl)(phenylcarbazolylphenyl)amine; (quarterphenylyl)(dibenzofuranylphenyl)(phenylcarbazolylphenyl)amine; (quarterphenylyl)(dibenzofuranylphenyl)(phenylcarbazolylphenyl)amine; (quarterphenylyl)bis(dibenzofuranylphenyl)amine; (quarterphenylyl)bis(dibenzofuranylphenyl)amine; (quarterphenylyl)(dibenzofuranylphenyl)(phenylcarbazolylphenyl)amine; (quarterphenylyl)(dibenzofuranylphenyl)(phenylcarbazolylphenyl)amine; (quarterphenylyl)(biphenylyl)(dibenzofuranylphenyl)amine; (quarterphenylyl)(biphenylyl)(dibenzofuranylphenyl)amine; (terphenylyl)(naphthylphenyl)(dibenzofuranylbiphenylyl)amine; (terphenylyl)(phenylnaphthyl)(dibenzofuranylbiphenylyl)amine; (terphenylyl)(naphthyl)(dibenzofuranylbiphenylyl)amine; (dimethylfluorenyl)bis(spirobifluorenyl)amine; (biphenylyl)bis(spirobifluorenyl)amine; (dimethylfluorenyl)bis(spirobifluorenyl)amine; (spirobifluorenyl)(biphenylyl)(dimethylfluorenyl)amine; (spirobifluorenyl)di(biphenylyl)amine; (spirobifluorenyl)bis(dimethylfluorenyl)amine; (spirobifluorenyl)(biphenylyl)(dimethylfluorenyl)amine; (spirobifluorenyl)(terphenylyl)(dimethylfluorenyl)amine; (spirobifluorenyl)(biphenylyl)(dimethylfluorenyl)amine; (spirobifluorenyl)bis(dimethylfluorenyl)amine; (spirobifluorenyl)(biphenylyl)(dimethylfluorenyl)amine; (spirobifluorenyl)di(biphenylyl)amine; (spirobifluorenylphenyl)di(biphenylyl)amine; (spirobifluorenyl)di(biphenylyl)amine; (spirobifluorenyl) (biphenylyl)(dibenzofuranyl)amine; (spirobifluorenyl)(biphenylyl)(dibenzofuranyl)amine; (spirobifluorenyl)(dibenzofuranyl)(dimethylfluorenyl)amine; (spirobifluorenyl)(biphenylyl)(phenylcarbazolyl)amine; (spirobifluorenyl)(diphenylfluorenyl)(dibenzothiophenylphenyl)amine; (spirobifluorenyl)(dimethylfluorenyl)(dibenzothiophenyl)amine; (spirobifluorenyl)(biphenylyl)(phenylcarbazolyl)amine; (spirobifluorenyl)(biphenylyl)(diphenylfluorenyl)amine; (spirobifluorenyl)(biphenylyl)(dibenzofuranylphenyl)amine; (spirobifluorenyl)(biphenylyl)(dibenzothiophenylphenyl)amine; (spirobifluorenyl)(biphenylyl)(diphenylfluorenyl)amine; (biphenylyl)bis(dibenzofuranylphenyl)amine; (terphenylyl)bis(dibenzofuranylphenyl)amine; (dimethylfluorenyl)bis(dibenzofuranylphenyl)amine; (terphenylyl)bis(dibenzofuranylphenyl)amine; (quarterphenylyl)bis(dibenzofuranylphenyl)amine; (diphenylfluorenyl)(dibenzofuranylphenyl)(phenyl)amine; (diphenylfluorenyl)(dibenzofuranylphenyl)(biphenylyl)amine; (diphenylfluorenyl)(dibenzofuranylphenyl)(biphenylyl)amine; (diphenylfluorenyl)(dibenzofuranylphenyl)(biphenylyl)amine; (diphenylfluorenyl)(dibenzofuranylphenyl)(terphenylyl)amine; (diphenylfluorenyl)(dibenzofuranylphenyl)(terphenylyl)amine; (diphenylfluorenyl)(dibenzofuranylphenyl)(terphenylyl)amine; (diphenylfluorenyl)(dibenzofuranylphenyl)(terphenylyl)amine; (diphenylfluorenyl)(dibenzofuranylphenyl)(naphthyl)amine; (diphenylfluorenyl)(dibenzofuranylphenyl)(naphthyl)amine; (diphenylfluorenyl)(dibenzofuranylphenyl)(naphthylphenyl)amine; (diphenylfluorenyl)(dibenzofuranylphenyl)(naphthylphenyl)amine; (diphenylfluorenyl)(dibenzofuranylphenyl)(dimethylfluorenyl)amine; (diphenylfluorenyl)(dibenzofuranylphenyl)(phenylmethylfluorenyl)amine; bis(diphenylfluorenyl)(dibenzofuranylphenyl)amine; (diphenylfluorenyl)(dibenzothiophenylphenyl)(phenyl)amine; (diphenylfluorenyl)(dibenzothiophenylphenyl)(biphenylyl)amine; (diphenylfluorenyl)(dibenzothiophenylphenyl)(biphenylyl)amine; (diphenylfluorenyl)(dibenzothiophenylphenyl)(terphenylyl)amine; (diphenylfluorenyl)(dibenzothiophenylphenyl)(naphthyl)amine; (diphenylfluorenyl)(dibenzothiophenylphenyl)(naphthyl)amine; (diphenylfluorenyl)(dibenzothiophenylphenyl)(naphthylphenyl)amine; (diphenylfluorenyl)(dibenzothiophenylphenyl)(naphthylphenyl)amine; (phenyl)(diphenylfluorenyl)(dibenzofuranylphenyl)amine; (biphenylyl)(diphenylfluorenyl)(dibenzofuranylphenyl)amine; (biphenylyl)(diphenylfluorenyl)(dibenzofuranylphenyl)amine; (naphthylphenyl)(diphenylfluorenyl)(dibenzofuranylphenyl)amine; (naphthylphenyl)(diphenylfluorenyl)(dibenzofuranylphenyl)amine; (dimethylfluorenyl)(diphenylfluorenyl)(dibenzofuranylphenyl)amine; bis(diphenylfluorenyl)(dibenzofuranylphenyl)amine; (phenyl)(diphenylfluorenyl)(dibenzothiophenylphenyl)amine; (biphenylyl)(diphenylfluorenyl)(dibenzothiophenylphenyl)amine; (biphenylyl)(diphenylfluorenyl)(dibenzothiophenylphenyl)amine; (terphenylyl)(diphenylfluorenyl)(dibenzothiophenylphenyl)amine; (naphthylphenyl)(diphenylfluorenyl)(dibenzothiophenylphenyl)amine; (naphthylphenyl)(diphenylfluorenyl)(dibenzothiophenylphenyl)amine; (dimethylfluorenyl)(diphenylfluorenyl)(dibenzothiophenylphenyl)amine; (biphenylyl)(diphenylfluorenyl)(carbazolylphenyl)amine; (biphenylyl)(diphenylfluorenyl)(carbazolylphenyl)amine; (terphenylyl)(diphenylfluorenyl)(carbazolylphenyl)amine; (naphthylphenyl)(diphenylfluorenyl)(carbazolylphenyl)amine; (naphthylphenyl)(diphenylfluorenyl)(carbazolylphenyl)amine; (dimethylfluorenyl)(diphenylfluorenyl)(carbazolylphenyl)amine; (biphenylyl)(dibenzofuranyl)(diphenylfluorenyl)amine; (dibenzofuranyl)(dimethylfluorenyl)(diphenylfluorenyl)amine; (biphenylyl)(dibenzothiophenyl)(diphenylfluorenyl)amine; (dimethylfluorenyl)(dibenzothiophenyl)(diphenylfluorenyl)amine; (biphenylyl)(dibenzofuranyl)(diphenylfluorenyl)amine; (biphenylyl)(dibenzofuranyl)(diphenylfluorenyl)amine; (dimethylfluorenyl)(dibenzofuranyl)(diphenylfluorenyl)amine; (biphenylyl)(dibenzothiophenyl)(diphenylfluorenyl)amine; (biphenylyl)(dibenzothiophenyl)(diphenylfluorenyl)amine; (dimethylfluorenyl)(dibenzothiophenyl)(diphenylfluorenyl)amine; bis(dibenzofuranylphenyl)(diphenylfluorenyl)amine; bis(dibenzofuranylphenyl)(diphenylfluorenyl)amine; (dibenzofuranylphenyl)(dibenzothiophenylphenyl)(diphenylfluorenyl)amine; (dibenzofuranylphenyl)(carbazolylphenyl)(diphenylfluorenyl)amine; bis(dibenzofuranylphenyl)(diphenylfluorenyl)amine; (dibenzofuranylphenyl)(dibenzothiophenylphenyl)(diphenylfluorenyl)amine; (dibenzofuranylphenyl)(carbazolylphenyl)(diphenylfluorenyl)amine; (dibenzothiophenylphenyl)(carbazolylphenyl)(diphenylfluorenyl)amine; (methylbiphenylyl)(dibenzofuranylphenyl)(diphenylfluorenyl)amine; (tert-butylbiphenylyl)(dibenzofuranylphenyl)(diphenylfluorenyl)amine; (dimethylbiphenylyl)(dibenzofuranylphenyl)(diphenylfluorenyl)amine; (dimethylbiphenylyl)(dibenzofuranylphenyl)(diphenylfluorenyl)amine; (methylbiphenylyl)(dibenzofuranylphenyl)(diphenylfluorenyl)amine; (tert-butylbiphenylyl)(dibenzofuranylphenyl)(diphenylfluorenyl)amine; (methoxybiphenylyl)(dibenzofuranylphenyl)(diphenylfluorenyl)amine; (phenyl)(dimethylfluorenyl)(diphenylfluorenyl)amine; (biphenylyl)(dimethylfluorenyl)(diphenylfluorenyl)amine; (biphenylyl)(dimethylfluorenyl)(diphenylfluorenyl)amine; (biphenylyl)(dimethylfluorenyl)(diphenylfluorenyl)amine; (terphenylyl)(dimethylfluorenyl)(diphenylfluorenyl)amine; (terphenylyl)(dimethylfluorenyl)(diphenylfluorenyl)amine; (terphenylyl)(dimethylfluorenyl)(diphenylfluorenyl)amine; (terphenylyl)(dimethylfluorenyl)(diphenylfluorenyl)amine; (terphenylyl)(dimethylfluorenyl)(diphenylfluorenyl)amine; (naphthyl)(dimethylfluorenyl)(diphenylfluorenyl)amine; (naphthyl)(dimethylfluorenyl)(diphenylfluorenyl)amine; (naphthylphenyl)(dimethylfluorenyl)(diphenylfluorenyl)amine; (naphthylphenyl)(dimethylfluorenyl)(diphenylfluorenyl)amine; bis(dimethylfluorenyl)(diphenylfluorenyl)amine; (phenylmethylfluorenyl)(dimethylfluorenyl)(diphenylfluorenyl)amine; (dimethylfluorenyl)bis(diphenylfluorenyl)amine; (terphenylyl)(dimethylfluorenyl)(diphenylfluorenyl)amine; (terphenylyl)(dimethylfluorenyl)(diphenylfluorenyl)amine; (fluorophenyl)(dimethylfluorenyl)(diphenylfluorenyl)amine; (methylphenyl)(dimethylfluorenyl)(diphenylfluorenyl)amine; (tert-butylphenyl)(dimethylfluorenyl)(diphenylfluorenyl)amine; (methylbiphenylyl)(dimethylfluorenyl)(diphenylfluorenyl)amine; (tert-butylbiphenylyl)(dimethylfluorenyl)(diphenylfluorenyl)amine; (dimethylbiphenylyl)(dimethylfluorenyl)(diphenylfluorenyl)amine; (dimethylbiphenylyl)(dimethylfluorenyl)(diphenylfluorenyl)amine; (methylbiphenylyl)(dimethylfluorenyl)(diphenylfluorenyl)amine; (tert-butylbiphenylyl)(dimethylfluorenyl)(diphenylfluorenyl)amine; (methoxybiphenylyl)(dimethylfluorenyl)(diphenylfluorenyl)amine; (phenyl)bis(diphenylfluorenyl)amine; (biphenylyl)bis(diphenylfluorenyl)amine; (biphenylyl)bis(diphenylfluorenyl)amine; (biphenylyl)bis(diphenylfluorenyl)amine; (terphenylyl)bis(diphenylfluorenyl)amine; (terphenylyl)bis(diphenylfluorenyl)amine; (terphenylyl)bis(diphenylfluorenyl)amine; (terphenylyl)bis(diphenylfluorenyl)amine; (terphenylyl)bis(diphenylfluorenyl)amine; (terphenylyl)bis(diphenylfluorenyl)amine; (naphthyl)bis(diphenylfluorenyl)amine; (naphthyl)bis(diphenylfluorenyl)amine; (naphthylphenyl)bis(diphenylfluorenyl)amine; (naphthylphenyl)bis(diphenylfluorenyl)amine; (dimethylfluorenyl)bis(diphenylfluorenyl)amine; (phenylmethylfluorenyl)bis(diphenylfluorenyl)amine; tris(diphenylfluorenyl)amine; (terphenylyl)bis(diphenylfluorenyl)amine; (terphenylyl)bis(diphenylfluorenyl)amine; (methylbiphenylyl)bis(diphenylfluorenyl)amine; (tert-butylbiphenylyl)bis(diphenylfluorenyl)amine; (dimethylbiphenylyl)bis(diphenylfluorenyl)amine; (dimethylbiphenylyl)bis(diphenylfluorenyl)amine; (methylbiphenylyl)bis(diphenylfluorenyl)amine; (tert-butylbiphenylyl)bis(diphenylfluorenyl)amine; (methoxybiphenylyl)bis(diphenylfluorenyl)amine; (phenyl)(dimethylfluorenyl)(diphenylfluorenyl)amine; (biphenylyl)(dimethylfluorenyl)(diphenylfluorenyl)amine; (biphenylyl)(dimethylfluorenyl)(diphenylfluorenyl)amine; (phenyl)(dibenzofuranylphenyl)(diphenylfluorenyl)amine; (biphenylyl)(dibenzofuranylphenyl)(diphenylfluorenyl)amine; (biphenylyl)(dibenzofuranylphenyl)(diphenylfluorenyl)amine; (biphenylyl)(dibenzofuranylphenyl)[di(biphenylyl)fluorenyl]amine; (biphenylyl)(dimethylfluorenyl)[di(methylphenyl)fluorenyl]amine; (biphenylyl)(dimethylfluorenyl)[di(methylphenyl)fluorenyl]amine; (biphenylyl)(dimethylfluorenyl)[di(tert-butylphenyl)fluorenyl]amine; (biphenylyl)(dibenzofuranylphenyl)[di(methylphenyl)fluorenyl]amine; (biphenylyl)(dibenzofuranylphenyl)[di(methylphenyl)fluorenyl]amine; (biphenylyl)(dibenzofuranylphenyl)[di(tert-butylphenyl)fluorenyl]amine; (biphenylyl)(dimethylfluorenyl)(methyldiphenylfluorenyl)amine; (biphenylyl)(dimethylfluorenyl)(methyldiphenylfluorenyl)amine; (biphenylyl)(dimethylfluorenyl)(tert-butyldiphenylfluorenyl)amine; (biphenylyl)(dimethylfluorenyl)(tert-butyldiphenylfluorenyl)amine; (biphenylyl)(dibenzofuranylphenyl)(methyldiphenylfluorenyl)amine; (biphenylyl)(dibenzofuranylphenyl)(methyldiphenylfluorenyl)amine; (biphenylyl)(dibenzofuranylphenyl)(tert-butyldiphenylfluorenyl)amine; (biphenylyl)(dibenzofuranylphenyl)(tert-butyldiphenylfluorenyl)amine; (biphenylyl)(dimethylfluorenyl)(triphenylfluorenyl)amine; (biphenylyl)(dimethylfluorenyl)(triphenylfluorenyl)amine; (biphenylyl)(dibenzofuranylphenyl)(triphenylfluorenyl)amine; (biphenylyl)(dibenzofuranylphenyl)(triphenylfluorenyl)amine; (biphenylyl)(methyldibenzofuranylphenyl)(diphenylfluorenyl)amine; (biphenylyl)(methyldibenzofuranylphenyl)(diphenylfluorenyl)amine; (biphenylyl)(phenyldibenzofuranylphenyl)(diphenylfluorenyl)amine; (biphenylyl)(phenyldibenzofuranylphenyl)(diphenylfluorenyl)amine; (biphenylyl)(methyldibenzofuranyl)(diphenylfluorenyl)amine; (biphenylyl)(methyldibenzofuranyl)(diphenylfluorenyl)amine; (biphenylyl)(phenyldibenzofuranyl)(diphenylfluorenyl)amine; (biphenylyl)(phenyldibenzofuranyl)(diphenylfluorenyl)amine; (dibenzofuranyl)bis(diphenylfluorenyl)amine; (dibenzothiophenyl)bis(diphenylfluorenyl)amine; (dibenzofuranylphenyl)bis(diphenylfluorenyl)amine; (dibenzothiophenylphenyl)bis(diphenylfluorenyl)amine; (dibenzofuranyl)bis(diphenylfluorenyl)amine; (dibenzothiophenyl)bis(diphenylfluorenyl)amine; (dibenzofuranylphenyl)bis(diphenylfluorenyl)amine; (dibenzothiophenylphenyl)bis(diphenylfluorenyl)amine; (dibenzothiophenyl)bis(diphenylfluorenyl)amine; (quarterphenylyl)bis(diphenylfluorenyl)amine; (dibenzothiophenylphenyl)(dimethylfluorenyl)(diphenylfluorenyl)amine; (quarterphenylyl)(dimethylfluorenyl)(diphenylfluorenyl)amine; (dibenzofuranylphenyl)(dimethylfluorenyl)(diphenylfluorenyl)amine; (dibenzothiophenylphenyl)(dimethylfluorenyl)(diphenylfluorenyl)amine; (dibenzofuranylphenyl)(dimethylfluorenyl)(diphenylfluorenyl)amine; (dibenzothiophenylphenyl)(dimethylfluorenyl)(diphenylfluorenyl)amine; (dibenzofuranylphenyl)(dimethylfluorenyl)(diphenylfluorenyl)amine; (dibenzothiophenylphenyl)(dimethylfluorenyl)(diphenylfluorenyl)amine; (dibenzofuranylphenyl)(dimethylfluorenyl)(diphenylfluorenyl)amine; (dibenzothiophenylphenyl)(dimethylfluorenyl)(diphenylfluorenyl)amine; (dibenzofuranylphenyl)(biphenylyl)(diphenylfluorenyl)amine; (dibenzothiophenylphenyl)(biphenylyl)(diphenylfluorenyl)amine; (dibenzofuranylphenyl)(biphenylyl)(diphenylfluorenyl)amine; (dibenzothiophenylphenyl)(biphenylyl)(diphenylfluorenyl)amine; (dibenzofuranylphenyl)(biphenylyl)(diphenylfluorenyl)amine; (dibenzothiophenylphenyl)(biphenylyl)(diphenylfluorenyl)amine; (dibenzofuranylphenyl)(biphenylyl)(diphenylfluorenyl)amine; (dibenzothiophenylphenyl)(biphenylyl)(diphenylfluorenyl)amine; (dibenzofuranylphenyl)(biphenylyl)(diphenylfluorenyl)amine; (dibenzothiophenylphenyl)(biphenylyl)(diphenylfluorenyl)amine; (dibenzofuranylphenyl)(biphenylyl)(diphenylfluorenyl)amine; (dibenzothiophenylphenyl)(biphenylyl)(diphenylfluorenyl)amine; (dibenzofuranylphenyl)(biphenylyl)(diphenylfluorenyl)amine; (dibenzothiophenylphenyl)(biphenylyl)(diphenylfluorenyl)amine; (dibenzothiophenylphenyl)(biphenylyl)(diphenylfluorenyl)amine; (dibenzofuranylphenyl)(biphenylyl)(diphenylfluorenyl)amine; (dibenzothiophenylphenyl)(biphenylyl)(diphenylfluorenyl)amine; (dibenzofuranylphenyl)(biphenylyl)(diphenylfluorenyl)amine; (dibenzothiophenylphenyl)(biphenylyl)(diphenylfluorenyl)amine; (dibenzofuranylphenyl)(biphenylyl)(diphenylfluorenyl)amine; (dibenzothiophenylphenyl)(biphenylyl)(diphenylfluorenyl)amine; (dibenzofuranylphenyl)(terphenylyl)(diphenylfluorenyl)amine; (dibenzothiophenylphenyl)(terphenylyl)(diphenylfluorenyl)amine; (dibenzothiophenylphenyl)(terphenylyl)(diphenylfluorenyl)amine; (dibenzofuranylphenyl)(terphenylyl)(diphenylfluorenyl)amine; (dibenzothiophenylphenyl)(terphenylyl)(diphenylfluorenyl)amine; (dibenzofuranylphenyl)(terphenylyl)(diphenylfluorenyl)amine; (dibenzothiophenylphenyl)(terphenylyl)(diphenylfluorenyl)amine; (phenyl)(dibenzofuranyl)(diphenylfluorenyl)amine; (phenyl)(dibenzothiophenyl)(diphenylfluorenyl)amine; (phenyl)(dibenzofuranyl)(diphenylfluorenyl)amine; (phenyl)(dibenzothiophenyl)(diphenylfluorenyl)amine; (phenyl)(dibenzofuranylphenyl)(diphenylfluorenyl)amine; (phenyl)(dibenzothiophenylphenyl)(diphenylfluorenyl)amine; (phenyl)(dibenzofuranylphenyl)(diphenylfluorenyl)amine; (phenyl)(dibenzothiophenylphenyl)(diphenylfluorenyl)amine; (phenyl)(dibenzofuranylphenyl)(diphenylfluorenyl)amine; (phenyl)(dibenzothiophenylphenyl)(diphenylfluorenyl)amine; (phenyl)(dibenzofuranylphenyl)(diphenylfluorenyl)amine; (phenyl)(dibenzothiophenylphenyl)(diphenylfluorenyl)amine; bis(dibenzofuranyl)(diphenylfluorenyl)amine; bis(dibenzothiophenyl)(diphenylfluorenyl)amine; (biphenyldimethylfluorenyl)(phenyl)(diphenylfluorenyl)amine; (naphthyldimethylfluorenyl)(phenyl)(diphenylfluorenyl)amine; (naphthyldimethylfluorenyl)(phenyl)(diphenylfluorenyl)amine; (biphenyldimethylfluorenyl)(biphenylyl)(diphenylfluorenyl)amine; (naphthyldimethylfluorenyl)(biphenylyl)(diphenylfluorenyl)amine; (naphthyldimethylfluorenyl)(biphenylyl)(diphenylfluorenyl)amine; (naphthyldimethylfluorenyl)(biphenylyl)(diphenylfluorenyl)amine; (phenyl)(dimethylfluorenylphenyl)(diphenylfluorenyl)amine; (phenyl)(dimethylfluorenylphenyl)(diphenylfluorenyl)amine; (biphenylyl)(dimethylfluorenylphenyl)(diphenylfluorenyl)amine; (biphenylyl)(dimethylfluorenylphenyl)(diphenylfluorenyl)amine; (biphenylyl)(dimethylfluorenylphenyl)(diphenylfluorenyl)amine; (biphenylyl)(dimethylfluorenylphenyl)(diphenylfluorenyl)amine; (biphenylyl)(dimethylfluorenylphenyl)(diphenylfluorenyl)amine; (biphenylyl)(dimethylfluorenylphenyl)(diphenylfluorenyl)amine; (dimethylfluorenylphenyl)bis(diphenylfluorenyl)amine; (dimethylfluorenylphenyl)bis(diphenylfluorenyl)amine; di(biphenylyl)(diphenylfluorenyl)amine; (biphenylyl)(methylbiphenylyl)(diphenylfluorenyl)amine; (methylphenyl)(biphenylyl)(diphenylfluorenyl)amine; di(biphenylyl)(diphenylfluorenyl)amine; bis(dimethylphenyl)(diphenylfluorenyl)amine; (biphenylyl)(terphenylyl)(diphenylfluorenyl)amine; bis(methylbiphenylyl)(diphenylfluorenyl)amine; (biphenylyl)(dimethylbiphenylyl)(diphenylfluorenyl)amine; (phenyl)(dibenzofuranylphenyl)(spirobifluorenyl)amine; (biphenylyl)(dibenzofuranylphenyl)(spirobifluorenyl)amine; (biphenylyl)(dibenzofuranylphenyl)(spirobifluorenyl)amine; (biphenylyl)(dibenzofuranylphenyl)(spirobifluorenyl)amine; (terphenylyl)(dibenzofuranylphenyl)(spirobifluorenyl)amine; (terphenylyl)(dibenzofuranylphenyl)(spirobifluorenyl)amine; (terphenylyl)(dibenzofuranylphenyl)(spirobifluorenyl)amine; (terphenylyl)(dibenzofuranylphenyl)(spirobifluorenyl)amine; (terphenylyl)(dibenzofuranylphenyl)(spirobifluorenyl)amine; (naphthyl)(dibenzofuranylphenyl)(spirobifluorenyl)amine; (naphthyl)(dibenzofuranylphenyl)(spirobifluorenyl)amine; (naphthylphenyl)(dibenzofuranylphenyl)(spirobifluorenyl)amine; (naphthylphenyl)(dibenzofuranylphenyl)(spirobifluorenyl)amine; (dimethylfluorenyl)(dibenzofuranylphenyl)(spirobifluorenyl)amine; (phenylmethylfluorenyl)(dibenzofuranylphenyl)(spirobifluorenyl)amine; (dibenzofuranylphenyl)(diphenylfluorenyl)(spirobifluorenyl)amine; (phenyl)(dibenzothiophenylphenyl)(spirobifluorenyl)amine; (biphenylyl)(dibenzothiophenylphenyl)(spirobifluorenyl)amine; (biphenylyl)(dibenzothiophenylphenyl)(spirobifluorenyl)amine; (terphenylyl)(dibenzothiophenylphenyl)(spirobifluorenyl)amine; (naphthyl)(dibenzothiophenylphenyl)(spirobifluorenyl)amine; (naphthyl)(dibenzothiophenylphenyl)(spirobifluorenyl)amine; (naphthylphenyl)(dibenzothiophenylphenyl)(spirobifluorenyl)amine; (naphthylphenyl)(dibenzothiophenylphenyl)(spirobifluorenyl)amine; (phenyl)(dibenzofuranylphenyl)(spirobifluorenyl)amine; (biphenylyl)(dibenzofuranylphenyl)(spirobifluorenyl)amine; (biphenylyl)(dibenzofuranylphenyl)(spirobifluorenyl)amine; (dibenzofuranylphenyl)(diphenylfluorenyl)(spirobifluorenyl)amine; (biphenylyl)(dibenzothiophenylphenyl)(spirobifluorenyl)amine; (biphenylyl)(dibenzothiophenylphenyl)(spirobifluorenyl)amine; (biphenylyl)(carbazolylphenyl)(spirobifluorenyl)amine; (biphenylyl)(carbazolylphenyl)(spirobifluorenyl)amine; (biphenylyl)(dibenzofuranyl)(spirobifluorenyl)amine; (biphenylyl)(dibenzofuranyl)(spirobifluorenyl)amine; (biphenylyl)(dibenzofuranyl)(spirobifluorenyl)amine; (dimethylfluorenyl)(dibenzofuranyl)(spirobifluorenyl)amine; (biphenylyl)(dibenzothiophenyl)(spirobifluorenyl)amine; (biphenylyl)(dibenzothiophenyl)(spirobifluorenyl)amine; (biphenylyl)(dibenzothiophenyl)(spirobifluorenyl)amine; (dimethylfluorenyl)(dibenzothiophenyl)(spirobifluorenyl)amine; (biphenylyl)(dibenzofuranyl)(spirobifluorenyl)amine; (biphenylyl)(dibenzofuranyl)(spirobifluorenyl)amine; (biphenylyl)(dibenzofuranyl)(spirobifluorenyl)amine; (dimethylfluorenyl)(dibenzofuranyl)(spirobifluorenyl)amine; (biphenylyl)(dibenzothiophenyl)(spirobifluorenyl)amine; (biphenylyl)(dibenzothiophenyl)(spirobifluorenyl)amine; (biphenylyl)(dibenzothiophenyl)(spirobifluorenyl)amine; (dimethylfluorenyl)(dibenzothiophenyl)(spirobifluorenyl)amine; bis(dibenzofuranylphenyl)(spirobifluorenyl)amine; (dibenzofuranylphenyl)(carbazolylphenyl)(spirobifluorenyl)amine; bis(dibenzofuranylphenyl)(spirobifluorenyl)amine; (dibenzofuranylphenyl)(carbazolylphenyl)(spirobifluorenyl)amine; (methylphenyl)(dibenzofuranylphenyl)(spirobifluorenyl)amine; (tert-butylphenyl)(dibenzofuranylphenyl)(spirobifluorenyl)amine; (methylbiphenylyl)(dibenzofuranylphenyl)(spirobifluorenyl)amine; (tert-butylbiphenylyl)(dibenzofuranylphenyl)(spirobifluorenyl)amine; (dimethylbiphenylyl)(dibenzofuranylphenyl)(spirobifluorenyl)amine; (dimethylbiphenylyl)(dibenzofuranylphenyl)(spirobifluorenyl)amine; (methylbiphenylyl)(dibenzofuranylphenyl)(spirobifluorenyl)amine; (tert-butylbiphenylyl)(dibenzofuranylphenyl)(spirobifluorenyl)amine; (phenyl)(dimethylfluorenyl)(spirobifluorenyl)amine; (biphenylyl)(dimethylfluorenyl)(spirobifluorenyl)amine; (biphenylyl)(dimethylfluorenyl)(spirobifluorenyl)amine; (biphenylyl)(dimethylfluorenyl)(spirobifluorenyl)amine; (terphenylyl)(dimethylfluorenyl)(spirobifluorenyl)amine; (terphenylyl)(dimethylfluorenyl)(spirobifluorenyl)amine; (terphenylyl)(dimethylfluorenyl)(spirobifluorenyl)amine; (terphenylyl)(dimethylfluorenyl)(spirobifluorenyl)amine; (terphenylyl)(dimethylfluorenyl)(spirobifluorenyl)amine; (naphthyl)(dimethylfluorenyl)(spirobifluorenyl)amine; (naphthyl)(dimethylfluorenyl)(spirobifluorenyl)amine; (naphthylphenyl)(dimethylfluorenyl)(spirobifluorenyl)amine; (naphthylphenyl)(dimethylfluorenyl)(spirobifluorenyl)amine; bis(dimethylfluorenyl)(spirobifluorenyl)amine; (phenylmethylfluorenyl)(dimethylfluorenyl)(spirobifluorenyl)amine; (diphenylfluorenyl)(dimethylfluorenyl)(spirobifluorenyl)amine; (terphenylyl)(dimethylfluorenyl)(spirobifluorenyl)amine; (terphenylyl)(dimethylfluorenyl)(spirobifluorenyl)amine; (methylphenyl)(dimethylfluorenyl)(spirobifluorenyl)amine; (tert-butylphenyl)(dimethylfluorenyl)(spirobifluorenyl)amine; (methylbiphenylyl)(dimethylfluorenyl)(spirobifluorenyl)amine; (tert-butylbiphenylyl)(dimethylfluorenyl)(spirobifluorenyl)amine; (dimethylbiphenylyl)(dimethylfluorenyl)(spirobifluorenyl)amine; (dimethylbiphenylyl)(dimethylfluorenyl)(spirobifluorenyl)amine; (methylbiphenylyl)(dimethylfluorenyl)(spirobifluorenyl)amine; (tert-butylbiphenylyl)(dimethylfluorenyl)(spirobifluorenyl)amine; (phenyl)(diphenylfluorenyl)(spirobifluorenyl)amine; (biphenylyl)(diphenylfluorenyl)(spirobifluorenyl)amine; (biphenylyl)(diphenylfluorenyl)(spirobifluorenyl)amine; (biphenylyl)(diphenylfluorenyl)(spirobifluorenyl)amine; (terphenylyl)(diphenylfluorenyl)(spirobifluorenyl)amine; (terphenylyl)(diphenylfluorenyl)(spirobifluorenyl)amine; (terphenylyl)(diphenylfluorenyl)(spirobifluorenyl)amine; (naphthyl)(diphenylfluorenyl)(spirobifluorenyl)amine; (naphthyl)(diphenylfluorenyl)(spirobifluorenyl)amine; (naphthylphenyl)(diphenylfluorenyl)(spirobifluorenyl)amine; (naphthylphenyl)(diphenylfluorenyl)(spirobifluorenyl)amine; (dimethylfluorenyl)(diphenylfluorenyl)(spirobifluorenyl)amine; (methylphenylfluorenyl)(diphenylfluorenyl)(spirobifluorenyl)amine; bis(diphenylfluorenyl)(spirobifluorenyl)amine; (terphenylyl)(diphenylfluorenyl)(spirobifluorenyl)amine; (terphenylyl)(diphenylfluorenyl)(spirobifluorenyl)amine; (methylphenyl)(diphenylfluorenyl)(spirobifluorenyl)amine; (tert-butylphenyl)(diphenylfluorenyl)(spirobifluorenyl)amine; (methylbiphenylyl)(diphenylfluorenyl)(spirobifluorenyl)amine; (tert-butylbiphenylyl)(diphenylfluorenyl)(spirobifluorenyl)amine; (dimethylbiphenylyl)(diphenylfluorenyl)(spirobifluorenyl)amine; (dimethylbiphenylyl)(diphenylfluorenyl)(spirobifluorenyl)amine; (methylbiphenylyl)(diphenylfluorenyl)(spirobifluorenyl)amine; (tert-butylbiphenylyl)(diphenylfluorenyl)(spirobifluorenyl)amine; (phenyl)(dimethylfluorenyl)(spirobifluorenyl)amine; (biphenylyl)(dimethylfluorenyl)(spirobifluorenyl)amine; (biphenylyl)(dimethylfluorenyl)(spirobifluorenyl)amine; (phenyl)(dibenzofuranylphenyl)(spirobifluorenyl)amine; (biphenylyl)(dibenzofuranylphenyl)(spirobifluorenyl)amine; (biphenylyl)(dibenzofuranylphenyl)(spirobifluorenyl)amine; (phenyl)(dimethylfluorenyl)(spirobifluorenyl)amine; (biphenylyl)(dimethylfluorenyl)(spirobifluorenyl)amine; (biphenylyl)(dimethylfluorenyl)(spirobifluorenyl)amine; (phenyl)(dimethylfluorenyl)(phenylspirobifluorenyl)amine; (biphenylyl)(dimethylfluorenyl)(phenylspirobifluorenyl)amine; (biphenylyl)(dimethylfluorenyl)(phenylspirobifluorenyl)amine; (biphenylyl)(dimethylfluorenyl)(phenylspirobifluorenyl)amine; (biphenylyl)(dimethylfluorenyl)(methylspirobifluorenyl)amine; (biphenylyl)(dimethylfluorenyl)(methylspirobifluorenyl)amine; (biphenylyl)(dimethylfluorenyl)(tert-butylspirobifluorenyl)amine; (biphenylyl)(dimethylfluorenyl)(tert-butylspirobifluorenyl)amine; (biphenylyl)(dimethylfluorenyl)(methylspirobifluorenyl)amine; (biphenylyl)(dimethylfluorenyl)(tert-butylspirobifluorenyl)amine; (biphenylyl)(dimethylfluorenyl)(methylspirobifluorenyl)amine; (biphenylyl)(dimethylfluorenyl)(methylspirobifluorenyl)amine; (biphenylyl)(dimethylfluorenyl)(tert-butylspirobifluorenyl)amine; (biphenylyl)(dimethylfluorenyl)(tert-butylspirobifluorenyl)amine; (biphenylyl)(dibenzofuranylphenyl)(methylspirobifluorenyl)amine; (biphenylyl)(dibenzofuranylphenyl)(methylspirobifluorenyl)amine; (biphenylyl)(dibenzofuranylphenyl)(tert-butylspirobifluorenyl)amine; (biphenylyl)(dibenzofuranylphenyl)(tert-butylspirobifluorenyl)amine; (biphenylyl)(dimethylfluorenyl)(phenylspirobifluorenyl)amine; (biphenylyl)(dimethylfluorenyl)(phenylspirobifluorenyl)amine; (biphenylyl)(dibenzofuranylphenyl)(phenylspirobifluorenyl)amine; (biphenylyl)(dibenzofuranylphenyl)(phenylspirobifluorenyl)amine; (biphenylyl)(methyldibenzofuranylphenyl)(spirobifluorenyl)amine; (biphenylyl)(methyldibenzofuranylphenyl)(spirobifluorenyl)amine; (biphenylyl)(phenyldibenzofuranylphenyl)(spirobifluorenyl)amine; (biphenylyl)(phenyldibenzofuranylphenyl)(spirobifluorenyl)amine; (biphenylyl)(methyldibenzofuranyl)(spirobifluorenyl)amine; (biphenylyl)(methyldibenzofuranyl)(spirobifluorenyl)amine; (biphenylyl)(phenyldibenzofuranyl)(spirobifluorenyl)amine; (biphenylyl)(phenyldibenzofuranyl)(spirobifluorenyl)amine; (dibenzofuranylphenyl)bis(spirobifluorenyl)amine; (dibenzothiophenylphenyl)bis(spirobifluorenyl)amine; (dibenzofuranyl)bis(spirobifluorenyl)amine; (dibenzothiophenyl)bis(spirobifluorenyl)amine; (dibenzofuranylphenyl)bis(spirobifluorenyl)amine; (dibenzothiophenylphenyl)bis(spirobifluorenyl)amine; (dibenzofuranylphenyl)bis(spirobifluorenyl)amine; (dibenzothiophenylphenyl)bis(spirobifluorenyl)amine; (dibenzofuranylphenyl)bis(spirobifluorenyl)amine; (dibenzothiophenylphenyl)bis(spirobifluorenyl)amine; (dibenzothiophenylphenyl)(dimethylfluorenyl)(spirobifluorenyl)amine; (dibenzofuranylphenyl)(dimethylfluorenyl)(spirobifluorenyl)amine; (dibenzothiophenylphenyl)(dimethylfluorenyl)(spirobifluorenyl)amine; (dibenzofuranylphenyl)(dimethylfluorenyl)(spirobifluorenyl)amine; (dibenzothiophenylphenyl)(dimethylfluorenyl)(spirobifluorenyl)amine; (dibenzofuranylphenyl)(dimethylfluorenyl)(spirobifluorenyl)amine; (dibenzothiophenylphenyl)(dimethylfluorenyl)(spirobifluorenyl)amine; (dibenzofuranylphenyl)(dimethylfluorenyl)(spirobifluorenyl)amine; (dibenzothiophenylphenyl)(dimethylfluorenyl)(spirobifluorenyl)amine; (dibenzofuranylphenyl)(biphenylyl)(spirobifluorenyl)amine; (dibenzothiophenylphenyl)(biphenylyl)(spirobifluorenyl)amine; (dibenzofuranylphenyl)(biphenylyl)(spirobifluorenyl)amine; (dibenzothiophenylphenyl)(biphenylyl)(spirobifluorenyl)amine; (dibenzofuranylphenyl)(biphenylyl)(spirobifluorenyl)amine; (dibenzothiophenylphenyl)(biphenylyl)(spirobifluorenyl)amine; (dibenzofuranylphenyl)(phenyl)(spirobifluorenyl)amine; (dibenzothiophenylphenyl)(phenyl)(spirobifluorenyl)amine; bis(dibenzofuranyl)(spirobifluorenyl)amine; bis(dibenzothiophenyl)(spirobifluorenyl)amine; (phenyl)(biphenyldimethylfluorenyl)(spirobifluorenyl)amine; (phenyl)(naphthyldimethylfluorenyl)(spirobifluorenyl)amine; (phenyl)(naphthyldimethylfluorenyl)(spirobifluorenyl)amine; (biphenylyl)(biphenyldimethylfluorenyl)(spirobifluorenyl)amine; (biphenylyl)(naphthyldimethylfluorenyl)(spirobifluorenyl)amine; (biphenylyl)(biphenyldimethylfluorenyl)(spirobifluorenyl)amine; (biphenylyl)(naphthyldimethylfluorenyl)(spirobifluorenyl)amine; (dimethylfluorenyl)(biphenyldimethylfluorenyl)(spirobifluorenyl)amine; (dimethylfluorenyl)(naphthyldimethylfluorenyl)(spirobifluorenyl)amine; (dimethylfluorenyl)(naphthyldimethylfluorenyl)(spirobifluorenyl)amine; (phenyl)(dimethylfluorenylphenyl)(spirobifluorenyl)amine; (biphenylyl)(dimethylfluorenylphenyl)(spirobifluorenyl)amine; (biphenylyl)(dimethylfluorenylphenyl)(spirobifluorenyl)amine; (biphenylyl)(dimethylfluorenylphenyl)(spirobifluorenyl)amine; (biphenylyl)(dimethylfluorenylphenyl)(spirobifluorenyl)amine; (terphenylyl)(dimethylfluorenylphenyl)(spirobifluorenyl)amine; (terphenylyl)(dimethylfluorenylphenyl)(spirobifluorenyl)amine; (dimethylfluorenyl)(dimethylfluorenylphenyl)(spirobifluorenyl)amine; (dimethylfluorenyl)(dimethylfluorenylphenyl)(spirobifluorenyl)amine; di(biphenylyl)(spirobifluorenyl)amine; (biphenylyl)(methylbiphenylyl)(spirobifluorenyl)amine; (methylphenyl)(biphenylyl)(spirobifluorenyl)amine; di(biphenylyl)(spirobifluorenyl)amine; (biphenylyl)(terphenylyl)(spirobifluorenyl)amine; bis(methylbiphenylyl)(spirobifluorenyl)amine; (biphenylyl)(dimethylbiphenylyl)(spirobifluorenyl)amine; (phenyl)(naphthyl)(biphenyldimethylfluorenyl)amine; (phenyl)(naphthyl)(biphenyldimethylfluorenyl)amine; (phenyl)(phenanthrenyl)(biphenyldimethylfluorenyl)amine; (phenyl)(biphenylyl)(biphenyldimethylfluorenyl)amine; (phenyl)(naphthylphenyl)(biphenyldimethylfluorenyl)amine; (phenyl)(naphthylphenyl)(biphenyldimethylfluorenyl)amine; (phenyl)(terphenylyl)(biphenyldimethylfluorenyl)amine; (phenyl)(terphenylyl)(biphenyldimethylfluorenyl)amine; bis(methylphenyl)(biphenyldimethylfluorenyl)amine; bis(methylphenyl)(biphenyldimethylfluorenyl)amine; (naphthyl)(methylphenyl)(biphenyldimethylfluorenyl)amine; (naphthyl)(methylphenyl)(biphenyldimethylfluorenyl)amine; (phenanthrenyl)(methylphenyl)(biphenyldimethylfluorenyl)amine; (biphenylyl)(methylphenyl)(biphenyldimethylfluorenyl)amine; (naphthylphenyl)(methylphenyl)(biphenyldimethylfluorenyl)amine; bis(methylphenyl)(biphenyldimethylfluorenyl)amine; (naphthyl)(methylphenyl)(biphenyldimethylfluorenyl)amine; (naphthyl)(methylphenyl)(biphenyldimethylfluorenyl)amine; (phenanthrenyl)(methylphenyl)(biphenyldimethylfluorenyl)amine; (biphenylyl)(methylphenyl)(biphenyldimethylfluorenyl)amine; (naphthylphenyl)(methylphenyl)(biphenyldimethylfluorenyl)amine; di(naphthyl)(biphenyldimethylfluorenyl)amine; di(naphthyl)(biphenyldimethylfluorenyl)amine; (naphthyl)(phenanthrenyl)(biphenyldimethylfluorenyl)amine; (naphthyl)(biphenylyl)(biphenyldimethylfluorenyl)amine; (naphthyl)(naphthylphenyl)(biphenyldimethylfluorenyl)amine; (naphthyl)(naphthylphenyl)(biphenyldimethylfluorenyl)amine; (naphthyl)(terphenylyl)(biphenyldimethylfluorenyl)amine; (naphthyl)(terphenylyl)(biphenyldimethylfluorenyl)amine; di(naphthyl)(biphenyldimethylfluorenyl)amine; (naphthyl)(phenanthrenyl)(biphenyldimethylfluorenyl)amine; (naphthyl)(biphenylyl)(biphenyldimethylfluorenyl)amine; (naphthyl)(naphthylphenyl)(biphenyldimethylfluorenyl)amine; (naphthyl)(naphthylphenyl)(biphenyldimethylfluorenyl)amine; (naphthyl)(terphenylyl)(biphenyldimethylfluorenyl)amine; (naphthyl)(terphenylyl)(biphenyldimethylfluorenyl)amine; di(phenanthrenyl)(biphenyldimethylfluorenyl)amine; (biphenylyl)(phenanthrenyl)(biphenyldimethylfluorenyl)amine; (naphthylphenyl)(phenanthrenyl)(biphenyldimethylfluorenyl)amine; (naphthylphenyl)(phenanthrenyl)(biphenyldimethylfluorenyl)amine; (terphenylyl)(phenanthrenyl)(biphenyldimethylfluorenyl)amine; (terphenylyl)(phenanthrenyl)(biphenyldimethylfluorenyl)amine; di(biphenylyl)(biphenyldimethylfluorenyl)amine; (phenyl)(dimethylfluorenyl)(biphenyldimethylfluorenyl)amine; (methylphenyl)(dimethylfluorenyl)(biphenyldimethylfluorenyl)amine; (methylphenyl)(dimethylfluorenyl)(biphenyldimethylfluorenyl)amine; (naphthyl)(dimethylfluorenyl)(biphenyldimethylfluorenyl)amine; (naphthyl)(dimethylfluorenyl)(biphenyldimethylfluorenyl)amine; (phenanthrenyl)(dimethylfluorenyl)(biphenyldimethylfluorenyl)amine; (biphenylyl)(dimethylfluorenyl)(biphenyldimethylfluorenyl)amine; (naphthylphenyl)(dimethylfluorenyl)(biphenyldimethylfluorenyl)amine; (naphthylphenyl)(dimethylfluorenyl)(biphenyldimethylfluorenyl)amine; bis(dimethylfluorenyl)(biphenyldimethylfluorenyl)amine; di(biphenylyl)(biphenyldimethylbenzofluorenyl)amine; di(biphenylyl)(biphenyldimethylbenzofluorenyl)amine; di(biphenylyl)(biphenyldimethyldibenzofluorenyl)amine; di(biphenylyl)(biphenyldiphenylfluorenyl)amine; di(biphenylyl)(biphenylphenylmethylfluorenyl)amine; (phenyl)(naphthyl)(biphenylspirobifluorenyl)amine; (phenyl)(biphenylyl)(biphenylspirobifluorenyl)amine; di(biphenylyl)(biphenylspirobifluorenyl)amine; di(phenyl)(biphenylspirobifluorenyl)amine; (phenyl)(methylphenyl)(biphenylspirobifluorenyl)amine; di(methylphenyl)(biphenylspirobifluorenyl)amine; (phenyl)(triphenylenyl)(biphenyldimethylfluorenyl)amine; (naphthyl)(triphenylenyl)(biphenyldimethylfluorenyl)amine; (phenyl)(naphthyl)(terphenyldimethylfluorenyl)amine; (phenyl)(naphthyl)(terphenyldimethylfluorenyl)amine; (phenyl)(biphenylyl)(terphenyldimethylfluorenyl)amine; (phenyl)(naphthylphenyl)(terphenyldimethylfluorenyl)amine; (phenyl)(naphthylphenyl)(terphenyldimethylfluorenyl)amine; di(phenyl)bis(phenylthiophenylphenyl)biphenyldiamine; di(biphenylyl)bis(phenylthiophenylphenyl)biphenyldiamine; di(phenyl)bis(phenylthiophenylbiphenylyl)biphenyldiamine; di(phenyl)bis(phenylthiophenyldimethylfluorenyl)biphenyldiamine; di(phenyl)bis(phenylthiophenylphenyl)terphenyldiamine; di(phenyl)bis(phenylthiophenylphenyl)dimethylfluorenyldiamine; bis(dimethylfluorenyl)bis(phenylthiophenylphenyl)biphenyldiamine; di(naphthyl)bis(phenylthiophenylphenyl)biphenyldiamine; di(phenyl)bis(naphthylthiophenylphenyl)biphenyldiamine; di(phenyl)bis(phenylthiophenylnaphthyl)biphenyldiamine; tri(biphenylyl)(phenylthiophenylphenyl)biphenyldiamine; (phenyl)di(biphenylyl)(phenylthiophenylphenyl)biphenyldiamine; di(biphenylyl)(naphthyl)(phenylthiophenylphenyl)biphenyldiamine; di(biphenylyl)bis(phenylthiophenylphenyl)biphenyldiamine; (phenylthiophenylphenyl)bis(diphenylaminobiphenylyl)amine; (phenylthiophenylphenyl)bis(diphenylaminoterphenylyl)amine; (phenylthiophenylphenyl)bis(diphenylaminodimethylfluorenyl)amine; (terphenylyl)bis[phenyl(phenylthiophenylphenyl)aminobiphenyl]amine; (phenylthiophenylphenyl)bis(phenylnaphthylaminobiphenylyl)amine; (phenyl)bis(diphenylaminophenyl)(phenylthiophenylphenyl)biphenyldiamine; bis(phenylthiophenylphenyl)bis(diphenylaminophenyl)biphenyldiamine; di(phenyl)bis[phenyl(phenylthiophenylphenyl)aminophenyl]biphenyldiamine; di(phenyl)bis[phenyl(phenylthiophenylphenyl)aminophenyl]terphenyldiamine; bis(phenylthiophenylphenyl)bis(diphenylaminobiphenylyl)biphenyldiamine; tris[phenyl(phenylthiophenylphenyl)aminobiphenyl]amine; tris[phenyl(phenylthiophenylphenyl)aminophenyl]amine; ( )( )( )amine; (phenyl)bis(diphenylaminophenyl)(phenylthiophenylphenyl)biphenyldiamine; (phenyl)(naphthyl)[(phenylcarbazolyl)dimethylfluorenyl]amine; (phenyl)(naphthyl)[(phenylcarbazolyl)biphenyl]amine; (phenyl)(naphthyl)[(phenylcarbazolyl)phenyl]amine; di(biphenylyl)[(phenylcarbazolyl)dimethylfluorenyl]amine; (phenyl)(naphthyl)[(diphenylcarbazolyl)dimethylfluorenyl]amine; di(biphenylyl)[(phenylcarbazolyl)phenyl]amine; (biphenylyl)(diphenylaminophenyl)[(phenylcarbazolyl)phenyl]amine; bis(diphenylaminophenyl)[(phenylcarbazolyl)phenyl]amine; (phenyl)(biphenylyl)[(phenylcarbazolyl)biphenyl]amine; (phenyl)(naphthyl)[(phenylcarbazolyl)terphenyl]amine; di(phenyl)[(phenylcarbazolyl)phenanthrenyl]amine; di(phenyl)[(phenylcarbazolyl)biphenyl]amine; di(biphenylyl)[(phenylcarbazolyl)naphthyl]amine; bis(naphthylphenyl)[(phenylcarbazolyl)phenyl]amine; (phenyl)(biphenylyl)[(diphenylcarbazolyl)phenyl]amine; di(biphenylyl)[(phenylcarbazolyl)thiophenylphenyl]amine; di(biphenylyl)[(phenylcarbazolyl)dibenzothiophenylphenyl]amine; di(biphenylyl)[(phenylcarbazolyl)furanylphenyl]amine; (biphenylyl)(phenanthrenyl)[(phenylcarbazolyl)phenyl]amine; di(biphenylyl)[(naphthylmethylcarbazolyl)phenyl]amine; (phenyl)(biphenylyl)[(phenylcarbazolyl)dibenzofuranylphenyl]amine; di(biphenylyl)[(phenylbiphenylcarbazolyl)phenyl]amine; (phenyl)(terphenylyl)[(phenylbiphenylcarbazolyl)phenyl]amine; (phenyl)(terphenylyl)[(phenylbiphenylcarbazolyl)phenyl]amine; bis(methylbiphenylyl)[(tert-butylphenylcarbazolyl)phenyl]amine; (biphenylyl)(diphenylaminobiphenylyl)[(phenylcarbazolyl)phenyl]amine; (phenyl)(phenyldimethylfluorenylaminobiphenylyl)[(phenylcarbazolyl)phenyl]amine; (phenyl)(phenylbiphenylaminobiphenylyl)[(phenylcarbazolyl)biphenyl]amine; (phenyl)[phenyl(diphenylaminophenyl)aminobiphenyl][(phenylcarbazolyl)phenyl]amine; (diphenylaminophenyl)(diphenylaminobiphenylyl)[(phenylcarbazolyl)phenyl]amine; (diphenylaminophenyl) [phenyl(dimethylfluorenyl)aminophenyl][(phenylcarbazolyl)phenyl]amine; bis(dibenzofuranylphenyl)[(phenylcarbazolyl)phenyl]amine; bis(phenylthiophenylphenyl)[(diphenylcarbazolyl)phenyl]amine; bis(dibenzothiophenylphenyl)[(phenylcarbazolyl)phenyl]amine; bis(dibenzothiophenylphenyl)[(biphenylcarbazolyl)phenyl]amine; di(terphenylyl)[(phenylcarbazolyl)phenyl]amine; di(biphenylyl)[(biphenylcarbazolyl)phenyl]amine; di(biphenylyl)[(naphthylcarbazolyl)phenyl]amine; (naphthyl)(biphenylyl)[(phenylcarbazolyl)phenyl]amine; tri(phenyl)[(phenylcarbazolyl)phenyl]biphenyldiamine; (phenyl)bis[(phenylcarbazolyl)phenyl]amine; (biphenylyl)bis[(phenylcarbazolyl)phenyl]amine; (naphthyl)bis[(phenylcarbazolyl)phenyl]amine; di(biphenylyl)[(phenylcarbazolyl)biphenyl]amine; di(phenyl)(carbazolylbiphenylyl)amine; (phenyl)(biphenylyl)(carbazolylbiphenylyl)amine; (phenyl)(biphenylyl)(carbazolylbiphenylyl)amine; (phenyl)(biphenylyl)(carbazolylbiphenylyl)amine; di(biphenylyl)(carbazolylbiphenylyl)amine; (phenyl)(naphthyl)(carbazolylbiphenylyl)amine; (naphthyl)(biphenylyl)(carbazolylbiphenylyl)amine; (phenyl)(naphthyl)(diphenylcarbazolylbiphenylyl)amine; di(biphenylyl)(carbazolyldimethylfluorenyl)amine; (phenyl)(naphthyl)(carbazolyldimethylfluorenyl)amine; (phenyl)(phenanthrenyl)(carbazolyldimethylfluorenyl)amine; (biphenylyl)(naphthyl)(carbazolyldimethylfluorenyl)amine; (phenyl)(naphthyl)(carbazolyldimethylfluorenyl)amine; di(naphthyl)(carbazolyldimethylfluorenyl)amine; (phenyl)(naphthyl)(diphenylcarbazolyldimethylfluorenyl)amine; (phenyl)(biphenylyl)(carbazolyldimethylfluorenyl)amine; (phenyl)(biphenylyl)(carbazolylbiphenylyl)amine; (phenyl)(biphenylyl)(carbazolylbiphenylyl)amine; (phenyl)(biphenylyl)(carbazolylbiphenylyl)amine; di(biphenylyl)(carbazolylbiphenylyl)amine; di(biphenylyl)(carbazolylbiphenylyl)amine; di(biphenylyl)(carbazolylbiphenylyl)amine; (phenyl)bis(carbazolylbiphenylyl)amine; (phenyl)bis(carbazolyldimethylfluorenyl)amine; phenylbis[(diphenylamino)phenyl]carbazole; phenylbis[(phenyl(naphthyl)amino)phenyl]carbazole; di(terphenylyl)(carbazolyldimethylfluorenyl)amine; bis(naphthylphenyl)(carbazolyldimethylfluorenyl)amine; di(biphenylyl)(diphenylcarbazolyldimethylfluorenyl)amine; (phenyl)(biphenylyl)(carbazolyldimethylfluorenyl)amine; (phenyl)(dimethylfluorenyl)(carbazolyldimethylfluorenyl)amine; (biphenylyl)(dimethylfluorenyl)(carbazolyldimethylfluorenyl)amine; bis(dimethylfluorenyl)(carbazolyldimethylfluorenyl)amine; (naphthyl)(dimethylfluorenyl)(carbazolyldimethylfluorenyl)amine; di(biphenylyl)(carbazolylphenyl)amine; di(biphenylyl)(carbazolylphenyl)amine; di(naphthyl)(carbazolylphenyl)amine; (phenyl)(naphthyl)(carbazolylbiphenylyl)amine; di(biphenylyl)(carbazolyldimethylbiphenylyl)amine; (phenyl)(naphthyl)(carbazolylterphenylyl)amine; (phenyl)(naphthyl)(carbazolylterphenylyl)amine; tetra(biphenylyl)biphenyldiamine; di(phenyl)di(naphthyl)biphenyldiamine; di(naphthyl)di(biphenylyl)biphenyldiamine; (phenyl)(biphenylyl)di(naphthyl)biphenyldiamine; (phenyl)(naphthyl)di(biphenylyl)biphenyldiamine; di(biphenylyl)di(terphenylyl)biphenyldiamine; di(biphenylyl)di(terphenylyl)biphenyldiamine; di(phenyl)di(terphenylyl)biphenyldiamine; di(phenyl)di(terphenylyl)biphenyldiamine; (phenyl)di(biphenylyl)(terphenylyl)biphenyldiamine; tri(biphenylyl)(terphenylyl)biphenyldiamine; (phenyl)di(biphenylyl)(terphenylyl)biphenyldiamine; tri(biphenylyl)(terphenylyl)biphenyldiamine; di(biphenylyl)bis(naphthylphenyl)biphenyldiamine; tri(biphenylyl)(naphthylphenyl)biphenyldiamine; di(biphenylyl)bis(naphthylphenyl)biphenyldiamine; tri(biphenylyl)(naphthylphenyl)biphenyldiamine; di(biphenylyl)bis(dimethylfluorenyl)biphenyldiamine; tetrakis(dimethylfluorenyl)biphenyldiamine; tetrakis(dimethylfluorenyl)dimethylfluorenyldiamine; di(phenyl)bis(dimethylfluorenyl)biphenyldiamine; di(phenyl)di(naphthyl)dimethylfluorenyldiamine; (phenyl)bis(diphenylaminobiphenylyl)amine; (phenyl)bis[phenyl(biphenylyl)aminobiphenyl]amine; (phenyl)bis[phenyl(naphthyl)aminobiphenyl]amine; (biphenylyl)bis[diphenylaminobiphenyl]amine; (biphenylyl)bis[diphenylaminoterphenyl]amine; di(phenyl)bis(diphenylaminobiphenylyl)biphenyldiamine; di(phenyl)bis[phenyl(naphthyl)aminophenyl]biphenyldiamine; di(naphthyl)bis(diphenylaminobiphenylyl)biphenyldiamine; di(phenyl)bis(phenylthiophenylphenyl)biphenyldiamine; (biphenylyl)(dimethylfluorenyl)(phenylcarbazolylphenyl)amine; (phenyl)(diphenylfluorenyl)(phenylcarbazolylphenyl)amine; (phenyl)(dimethylfluorenyl)(phenylcarbazolylbiphenylyl)amine; (phenyl)(terphenylyl)(biphenyldimethylfluorenyl)amine; di(biphenylyl)(biphenyldimethylfluorenyl)amine; di(biphenylyl)(naphthyldimethylfluorenyl)amine; (biphenylyl)(dimethylfluorenyl)(phenylcarbazolylphenyl)amine; di(biphenylyl)(phenylcarbazolylphenyl)amine; (biphenylyl)(terphenylyl)(phenylcarbazolylphenyl)amine; (naphthyl)(dimethylfluorenyl)(phenylcarbazolylbiphenylyl)amine; (biphenylyl)(dibenzothiophenyl)(phenylcarbazolylbiphenylyl)amine; bis(naphthylphenyl)(phenanthrenylphenyl)amine; (biphenylyl)(phenylnaphthylphenyl)(naphthylphenyl)amine; (biphenylyl)(dibenzofuranylbiphenylyl)(naphthylphenyl)amine; di(biphenylyl)(dibenzofuranylbiphenylyl)amine; di(biphenylyl)(dibenzothiophenylbiphenylyl)amine; di(biphenylyl)(triphenylsilylbiphenylyl)amine; di(biphenylyl)(triphenylsilylbiphenylyl)amine; (phenyl)(diphenylfluorenyl)(triphenylsilylbiphenylyl)amine; (biphenylyl)bis(naphthylphenyl)amine; tris(naphthylphenyl)amine; (phenyl)bis(carbazolylbiphenylyl)amine; bis(dibenzofuranylphenyl)(carbazolylbiphenylyl)amine; (biphenylyl)(dibenzofuranylphenyl)(carbazolylbiphenylyl)amine; (terphenylyl)(dibenzofuranylphenyl)(carbazolylbiphenylyl)amine; (biphenylyl)(dibenzofuranylphenyl)(carbazolylbiphenylyl)amine; (biphenylyl)(dimethylfluorenyl)(carbazolylbiphenylyl)amine; (biphenylyl)(dibenzofuranyl)(carbazolylbiphenylyl)amine; (phenyl)(biphenylyl)(carbazolylbiphenylyl)amine; di(biphenylyl)(carbazolylbiphenylyl)amine; (phenyl)(diphenylfluorenyl)(triphenylsilylbiphenylyl)amine; (dimethylfluorenyl)(biphenylyl)(carbazolylphenyl)amine; (biphenylyl)bis(diphenylfluorenyl)amine; (biphenylyl)bis(spirobifluorenyl)amine

### (Group 34A)

phenyl(naphthylphenyl)anthracene; phenyl(biphenylyl)anthracene; phenyl(naphthylphenyl)anthracene; phenyl(biphenylyl)anthracene; diphenylanthracene; phenyl(naphthyl)anthracene; phenyl(phenanthrenyl)anthracene; phenyl(naphthylphenyl)anthracene; phenyl(phenylnaphthyl)anthracene; phenyl(biphenylyl)anthracene; phenyl(naphthylphenyl)anthracene; phenyl(phenylnaphthyl)anthracene; phenyl(naphthyl)anthracene-d₈; phenyl(naphthylphenyl)anthracene-d₈; phenyl(naphthyl)anthracene-d₈; phenyl(naphthylphenyl)anthracene-d₈; phenyl(naphthylphenyl)anthracene-d₈; phenyl(biphenylyl)anthracene-d₈; phenyl(phenylnaphthyl)anthracene-d₈; diphenyl(naphthyl)anthracene; diphenyl(naphthylphenyl)anthracene; dinaphthyl(phenyl)anthracene; diphenyl(naphthyl)anthracene; diphenyl(biphenylyl)anthracene; phenyl(naphthyl)(naphthylphenyl)anthracene; phenyl(naphthyl)(naphthylphenyl)anthracene; diphenyl(naphthylphenyl)anthracene; diphenyl(naphthylphenyl)anthracene; dinaphthyl(phenyl)anthracene; phenyl(naphthyl)(biphenylyl)anthracene; diphenyl(naphthylphenyl)anthracene-d₇; diphenyl(naphthylphenyl)anthracene-d₇; naphthyl(naphthylphenyl)anthracene; naphthyl(biphenylyl)anthracene; dinaphthylanthracene; naphthyl(naphthylphenyl)anthracene; naphthyl(biphenylyl)anthracene; dinaphthylanthracene; naphthyl(biphenylyl)anthracene; naphthyl(phenanthrenyl)anthracene; naphthyl(naphthylphenyl)anthracene; naphthyl(phenylnaphthyl)anthracene; naphthyl(naphthylphenyl)anthracene; phenyl(naphthyl)anthracene; naphthyl(phenylnaphthyl)anthracene; naphthyl(naphthylphenyl)anthracene-d₈; dinaphthylanthracene-d₈; naphthyl(naphthylphenyl)anthracene-d₈; dinaphthylanthracene-d₈; naphthyl(naphthylphenyl)anthracene-d₈; naphthyl(biphenylyl)anthracene-d₈; naphthyl(phenylnaphthyl)anthracene-d₈; phenyl(naphthyl)anthracene-d₈; dinaphthyl(phenyl)anthracene; dinaphthyl(phenyl)anthracene; phenyl(naphthyl)(naphthylphenyl)anthracene; phenyl(naphthyl)(naphthylphenyl)anthracene; trinaphthylanthracene; phenyl(naphthyl)(biphenylyl)anthracene; trinaphthylanthracene; dinaphthyl(biphenylyl)anthracene; phenyl(naphthyl)(naphthylphenyl)anthracene; dinaphthyl(naphthylphenyl)anthracene; phenyl(naphthyl)(naphthylphenyl)anthracene; dinaphthyl(naphthylphenyl)anthracene; dinaphthyl(naphthylphenyl)anthracene; phenyl(naphthyl)(naphthylphenyl)anthracene-d₇; phenyl(naphthyl)(naphthylphenyl)anthracene-d₇; naphthyl(naphthylphenyl)anthracene; naphthyl(biphenylyl)anthracene; dinaphthylanthracene; naphthyl(naphthylphenyl)anthracene; naphthyl(biphenylyl)anthracene; naphthyl(phenanthrenyl)anthracene; naphthyl(biphenylyl)anthracene; naphthyl(naphthylphenyl)anthracene; naphthyl(naphthylphenyl)anthracene; naphthyl(phenylnaphthyl)anthracene; naphthyl(naphthylphenyl)anthracene-d₈; naphthyl(naphthylphenyl)anthracene-d₈; naphthyl(naphthylphenyl)anthracene-d₈; naphthyl(biphenylyl)anthracene-d₈; naphthyl(phenylnaphthyl)anthracene-d₈; dinaphthyl(phenyl)anthracene; phenyl(naphthyl)(naphthylphenyl)anthracene; phenyl(naphthyl)(naphthylphenyl)anthracene; phenyl(naphthyl)(biphenylyl)anthracene; trinaphthylanthracene; phenyl(naphthyl)(naphthylphenyl)anthracene; phenyl(naphthyl)(naphthylphenyl)anthracene; dinaphthyl(naphthylphenyl)anthracene; dinaphthyl(naphthylphenyl)anthracene; dinaphthyl(naphthylphenyl)anthracene; dinaphthyl(naphthylphenyl)anthracene; phenyl(naphthyl)(naphthylphenyl)anthracene-d₇; phenyl(naphthyl)(naphthylphenyl)anthracene-d₇; phenanthrenyl(biphenylyl)anthracene; phenanthrenyl(naphthylphenyl)anthracene; phenanthrenyl(biphenylyl)anthracene; phenyl(phenanthrenyl)anthracene; phenanthrenyl(naphthylphenyl)anthracene; phenanthrenyl(biphenylyl)anthracene; diphenanthrenylanthracene; phenanthrenyl(naphthylphenyl)anthracene; phenanthrenyl(naphthylphenyl)anthracene-d₈; phenyl(phenanthrenyl)anthracene-d₈; phenanthrenyl(naphthylphenyl)anthracene-d₈; phenanthrenyl(naphthylphenyl)anthracene-d₈; (phenylnaphthyl)(naphthylphenyl)anthracene; biphenyl(phenylnaphthyl)anthracene; (phenylnaphthyl)(naphthylphenyl)anthracene; biphenyl(phenylnaphthyl)anthracene; (phenylnaphthyl)(naphthylphenyl)anthracene; (phenylnaphthyl)(naphthylphenyl)anthracene; (phenylnaphthyl)(naphthylphenyl)anthracene; (phenylnaphthyl)(naphthylphenyl)anthracene-d₈; (phenylnaphthyl)(naphthylphenyl)anthracene-d₈; (phenylnaphthyl)(naphthylphenyl)anthracene-d₈; benzophenanthrenyl(naphthylphenyl)anthracene; biphenyl[(benzophenanthrenyl)phenyl]anthracene; benzophenanthrenyl(biphenylyl)anthracene; benzophenanthrenyl(naphthyl)anthracene; [(benzophenanthrenyl)phenyl](naphthyl)anthracene; [(benzophenanthrenyl)phenyl](naphthylphenyl)anthracene; benzophenanthrenyl(naphthylphenyl)anthracene; benzophenanthrenyl(biphenylyl)anthracene; [(benzophenanthrenyl)phenyl](biphenylyl)anthracene; benzophenanthrenyl(phenyl)anthracene; [(benzophenanthrenyl)phenyl](phenyl)anthracene; benzophenanthrenyl(biphenylyl)anthracene; [(benzophenanthrenyl)phenyl](naphthyl)anthracene; benzophenanthrenyl(phenylnaphthyl)anthracene; benzophenanthrenyl(naphthyl)anthracene; [(benzophenanthrenyl)phenyl](biphenylyl)anthracene; [(benzophenanthrenyl)phenyl](naphthylphenyl)anthracene; benzophenanthrenyl(naphthylphenyl)anthracene; benzophenanthrenyl(naphthylphenyl)anthracene; benzotriphenylenyl(naphthyl)anthracene; benzotriphenylenyl(phenyl)anthracene; benzotriphenylenyl(biphenylyl)anthracene; benzotriphenylenyl(phenylnaphthyl)anthracene; benzotriphenylenyl(naphthylphenyl)anthracene; benzotriphenylenyl(phenanthrenyl)anthracene; benzotriphenylenyl(naphthylphenyl)anthracene; benzotriphenylenyl(naphthylphenyl)anthracene; benzotriphenylenyl(biphenylyl)anthracene; benzotriphenylenyl(naphthylphenyl)anthracene; benzotriphenylenyl(biphenylyl)anthracene; phenyl(benzanthracenyl)anthracene; naphthyl(benzanthracenyl)anthracene; (naphthylphenyl)(benzanthracenyl)anthracene; biphenyl(benzanthracenyl)anthracene; naphthyl(benzanthracenyl)anthracene; (naphthylphenyl)(benzanthracenyl)anthracene; (phenylnaphthyl)(benzanthracenyl)anthracene; biphenyl(benzanthracenyl)anthracene; (naphthylphenyl)(benzanthracenyl)anthracene; (naphthylphenyl)(benzanthracenyl)anthracene; biphenyl(benzanthracenyl)anthracene; naphthyl(benzophenanthrenyl)anthracene-d₈; naphthyl(benzophenanthrenyl)anthracene-d₈; phenyl(benzophenanthrenyl)anthracene-d₈; naphthyl[(benzophenanthrenyl)phenyl]anthracene-d₈; (naphthylphenyl)(benzophenanthrenyl)anthracene-d₈; (naphthylphenyl)[(benzophenanthrenyl)phenyl]anthracene-d₈; (naphthylphenyl)(benzophenanthrenyl)anthracene-d₈; naphthyl[(benzophenanthrenyl)phenyl]anthracene-d₈; (phenylnaphthyl)(benzophenanthrenyl)anthracene-d₈; (naphthylphenyl)(benzophenanthrenyl)anthracene-d₈; biphenyl(benzophenanthrenyl)anthracene-d₈; naphthyl(benzotriphenylenyl)anthracene-d₈; naphthyl(benzotriphenylenyl)anthracene-d₈; phenyl(benzotriphenylenyl)anthracene-d₈; phenyl(benzanthracenyl)anthracene-d₈; naphthyl(benzanthracenyl)anthracene-d₈; naphthyl(benzanthracenyl)anthracene-d₈; (naphthylphenyl)(benzanthracenyl)anthracene-d₈; biphenyl(dimethylfluorenyl)anthracene; naphthyl(dimethylfluorenyl)anthracene; (naphthylphenyl)(dimethylfluorenyl)anthracene; biphenyl(dimethylfluorenyl)anthracene; phenyl(dimethylfluorenyl)anthracene; (phenylnaphthyl)(dimethylfluorenyl)anthracene; naphthyl(dimethylfluorenyl)anthracene; phenanthrenyl(dimethylfluorenyl)anthracene; biphenyl(dimethylfluorenyl)anthracene; (naphthylphenyl)(dimethylfluorenyl)anthracene; (naphthylphenyl)(dimethylfluorenyl)anthracene; naphthyl(dimethylfluorenyl)anthracene-d₈; phenyl(dimethylfluorenyl)anthracene-d₈; biphenyl(dimethylfluorenyl)anthracene-d₈; naphthyl(dimethylfluorenyl)anthracene-d₈; diphenyl(dimethylfluorenyl)anthracene; biphenyl[(dimethylfluorenyl)phenyl]anthracene; (phenylnaphthyl)[(dimethylfluorenyl)phenyl]anthracene; naphthyl[(dimethylfluorenyl)phenyl]anthracene; phenyl[(dimethylfluorenyl)phenyl]anthracene; biphenyl[(dimethylfluorenyl)phenyl]anthracene; (phenylnaphthyl)[(dimethylfluorenyl)phenyl]anthracene; naphthyl[(dimethylfluorenyl)phenyl]anthracene; biphenyl[(dimethylfluorenyl)phenyl]anthracene; phenanthrenyl[(dimethylfluorenyl)phenyl]anthracene; (naphthylphenyl)[(dimethylfluorenyl)phenyl]anthracene; (naphthylphenyl)[(dimethylfluorenyl)phenyl]anthracene; phenyl[(dimethylfluorenyl)phenyl]anthracene-d₈; naphthyl[(dimethylfluorenyl)phenyl]anthracene-d₈; naphthyl[(dimethylfluorenyl)phenyl]anthracene-d₈; biphenyl[(dimethylfluorenyl)phenyl]anthracene-d₈; diphenyl[(dimethylfluorenyl)phenyl]anthracene; naphthyl(naphthylphenyl)anthracene-d₂₆; naphthyl(naphthylphenyl)anthracene-d₂₆; naphthyl(naphthylphenyl)anthracene-d₂₆; naphthyl(naphthylphenyl)anthracene-d₂₆; phenyl(naphthylphenyl)anthracene-d₂₄; dinaphthylanthracene-d₂₂; dinaphthylanthracene-d₂₂; phenyl(naphthobenzofuranyl)anthracene; biphenyl(naphthobenzofuranyl)anthracene; biphenyl(naphthobenzofuranyl)anthracene; phenyl(phenylnaphthobenzofuranyl)anthracene; phenyl(phenylnaphthobenzofuranyl)anthracene; phenyl(phenylnaphthobenzofuranyl)anthracene; phenyl(phenylnaphthobenzofuranyl)anthracene; phenyl[(naphthobenzofuranyl)phenyl]anthracene; phenyl[(naphthobenzofuranyl)phenyl]anthracene; biphenyl[(naphthobenzofuranyl)phenyl]anthracene; biphenyl[(naphthobenzofuranyl)phenyl]anthracene; (dimethylfluorenyl)(naphthobenzofuranyl)anthracene; (dimethylfluorenyl)(naphthobenzofuranyl)anthracene; (dimethylfluorenyl)(naphthobenzofuranyl)anthracene; [(dimethylfluorenyl)phenyl](naphthobenzofuranyl)anthracene; [(dibenzofuranyl)phenyl](naphthobenzofuranyl)anthracene; [(dimethylfluorenyl)phenyl](naphthobenzofuranyl)anthracene; (dimethylfluorenyl)[(naphthobenzofuranyl)phenyl]anthracene; (dimethylfluorenyl)[(naphthobenzofuranyl)phenyl]anthracene; [(dibenzofuranyl)phenyl][(naphthobenzofuranyl)phenyl]anthracene; [(dibenzofuranyl)phenyl][(naphthobenzofuranyl)phenyl]anthracene; (dibenzofuranyl)(naphthobenzofuranyl)anthracene; (dibenzofuranyl)(naphthobenzofuranyl)anthracene; (dibenzofuranyl)(naphthobenzofuranyl)anthracene; [(dibenzofuranyl)phenyl](naphthobenzofuranyl)anthracene; [(dimethylfluorenyl)phenyl](naphthobenzofuranyl)anthracene; (naphthylphenyl)(naphthobenzofuranyl)anthracene; (dibenzofuranyl)[(naphthobenzofuranyl)phenyl]anthracene; (dibenzofuranyl)[(naphthobenzofuranyl)phenyl]anthracene; [(dibenzofuranyl)phenyl][(naphthobenzofuranyl)phenyl]anthracene; [(dibenzofuranyl)phenyl][(naphthobenzofuranyl)phenyl]anthracene; (benzanthracenyl)(naphthobenzofuranyl)anthracene; (benzophenanthrenyl)[(naphthobenzofuranyl)phenyl]anthracene; (phenylcarbazolyl)(naphthobenzofuranyl)anthracene; [(carbazolyl)phenyl](naphthobenzofuranyl)anthracene; (phenanthrenyl)(naphthobenzofuranyl)anthracene; (phenanthrenyl)[(naphthobenzofuranyl)phenyl]anthracene; (phenanthrenyl)[(naphthobenzofuranyl)phenyl]anthracene; (spirobifluorenyl)[(naphthobenzofuranyl)phenyl]anthracene; (spirobifluorenyl)[(naphthobenzofuranyl)phenyl]anthracene; (benzophenanthrenyl)(naphthobenzofuranyl)anthracene; (benzotriphenylenyl)(naphthobenzofuranyl)anthracene; (triphenylenyl)(naphthobenzofuranyl)anthracene; (benzotriphenylenyl)(naphthobenzofuranyl)anthracene; (benzophenanthrenyl)(naphthobenzofuranyl)anthracene; (phenanthrenyl)(naphthobenzofuranyl)anthracene; (dibenzothiophenyl)(naphthobenzofuranyl)anthracene; (benzophenanthrenyl)[(naphthobenzofuranyl)phenyl]anthracene; (diphenylfluorenyl)[(naphthobenzofuranyl)phenyl]anthracene; (diphenylfluorenyl)[(naphthobenzofuranyl)phenyl]anthracene; (phenyl-d₅)(naphthobenzofuranyl)anthracene; (biphenyl-d₆)(naphthobenzofuranyl)anthracene; (biphenyl-d₅)(naphthobenzofuranyl)anthracene; (fluorophenyl)(naphthobenzofuranyl)anthracene; (cyanophenyl)(naphthobenzofuranyl)anthracene; di(naphthobenzofuranyl)anthracene; [(naphthobenzofuranyl)phenyl](naphthobenzofuranyl)anthracene; [(naphthobenzofuranyl)phenyl](naphthobenzofuranyl)anthracene; bis[(naphthobenzofuranyl)phenyl]anthracene; bis[(naphthobenzofuranyl)phenyl]anthracene; [(phenylnaphthyl)phenyl](naphthobenzofuranyl)anthracene; (phenylphenanthrenyl)(naphthobenzofuranyl)anthracene; (phenylbenzocarbazolyl)(naphthobenzofuranyl)anthracene; di(naphthobenzofuranyl)anthracene; [(triphenylsilyl)phenyl](naphthobenzofuranyl)anthracene; di(naphthobenzofuranyl)anthracene; [(naphthobenzofuranyl)phenyl](naphthobenzofuranyl)anthracene; (dimethylbenzofluorenyl)(naphthobenzofuranyl)anthracene; [(benzanthracenyl)phenyl](naphthobenzofuranyl)anthracene; bis[(naphthobenzofuranyl)phenyl]anthracene; bis[(naphthobenzofuranyl)phenyl]anthracene; (terphenylyl)(naphthobenzofuranyl)anthracene; (terphenylyl)(naphthobenzofuranyl)anthracene; (terphenylyl)(naphthobenzofuranyl)anthracene; phenyl(phenylnaphthobenzofuranyl)anthracene; phenyl(phenylnaphthobenzofuranyl)anthracene; phenyl(phenylnaphthobenzofuranyl)anthracene; [(naphthobenzofuranyl)phenyl](biphenylyl)anthracene; [(naphthobenzofuranyl)phenyl](biphenylyl)anthracene; [(naphthobenzofuranyl)phenyl](biphenylyl)anthracene; [(naphthobenzofuranyl)phenyl](biphenylyl)anthracene; naphthyl(naphthobenzofuranyl)anthracene; naphthyl(naphthobenzofuranyl)anthracene; (phenylnaphthyl)(naphthobenzofuranyl)anthracene; (phenylnaphthyl)(naphthobenzofuranyl)anthracene; (phenylnaphthyl)(naphthobenzofuranyl)anthracene; (phenylnaphthyl)(naphthobenzofuranyl)anthracene; (phenylnaphthyl)(naphthobenzofuranyl)anthracene; (phenylnaphthyl)(naphthobenzofuranyl)anthracene; (phenylnaphthyl)(naphthobenzofuranyl)anthracene; naphthyl[(naphthobenzofuranyl)phenyl]anthracene; naphthyl[(naphthobenzofuranyl)phenyl]anthracene; (phenylnaphthyl)(naphthobenzofuranyl)anthracene; (phenylnaphthyl)(naphthobenzofuranyl)anthracene; (phenylnaphthyl)(naphthobenzofuranyl)anthracene; (phenylnaphthyl)(naphthobenzofuranyl)anthracene; (phenylnaphthyl)(naphthobenzofuranyl)anthracene; (phenylnaphthyl)(naphthobenzofuranyl)anthracene; naphthyl[(naphthobenzofuranyl)phenyl]anthracene; naphthyl[(naphthobenzofuranyl)phenyl]anthracene; (naphthylphenyl)(naphthobenzofuranyl)anthracene; (naphthylphenyl)(naphthobenzofuranyl)anthracene; (phenanthrenyl)(naphthobenzofuranyl)anthracene; phenyl(naphthobenzothiophenyl)anthracene; biphenyl(naphthobenzothiophenyl)anthracene; biphenyl(naphthobenzothiophenyl)anthracene; naphthyl(naphthobenzothiophenyl)anthracene; naphthyl(naphthobenzothiophenyl)anthracene; phenyl(phenylnaphthobenzothiophenyl)anthracene; phenyl(phenylnaphthobenzothiophenyl)anthracene; phenyl[(naphthobenzothiophenyl)phenyl]anthracene; phenyl[(naphthobenzothiophenyl)phenyl]anthracene; biphenyl[(naphthobenzothiophenyl)phenyl]anthracene; biphenyl[(naphthobenzothiophenyl)phenyl]anthracene; naphthyl[(naphthobenzothiophenyl)phenyl]anthracene; naphthyl[(naphthobenzothiophenyl)phenyl]anthracene; terphenyl(naphthobenzothiophenyl)anthracene; terphenyl(naphthobenzothiophenyl)anthracene; terphenyl(naphthobenzothiophenyl)anthracene; (phenylnaphthyl)(naphthobenzothiophenyl)anthracene; (naphthylphenyl)(naphthobenzothiophenyl)anthracene; (naphthylphenyl)(naphthobenzothiophenyl)anthracene; biphenyl[(naphthobenzothiophenyl)phenyl]anthracene; biphenyl[(naphthobenzothiophenyl)phenyl]anthracene; biphenyl[(naphthobenzothiophenyl)phenyl]anthracene; biphenyl[(naphthobenzothiophenyl)phenyl]anthracene; (phenylnaphthyl)[(naphthobenzothiophenyl)phenyl]anthracene; (phenylnaphthyl)[(naphthobenzothiophenyl)phenyl]anthracene; (fluorophenyl)(naphthobenzofuranyl)anthracene; (fluorophenyl)(naphthobenzofuranyl)anthracene; (fluorophenyl)(naphthobenzofuranyl)anthracene; (fluorobiphenylyl)(naphthobenzofuranyl)anthracene; (cyanobiphenylyl)(naphthobenzofuranyl)anthracene; (terphenylyl)(naphthobenzofuranyl)anthracene-d₃₀; (binaphthyl)(naphthobenzofuranyl)anthracene-d₃₀; (phenylnaphthyl)(naphthobenzofuranyl)anthracene-d₂₈; phenyl(naphthobenzofuranyl)anthracene-d₂₂; (terphenylyl)(naphthobenzofuranyl)anthracene-d₃₀; (biphenylyl)(naphthobenzofuranyl)anthracene-d₂₆; (phenylnaphthyl)(naphthobenzofuranyl)anthracene-d₂₈; (naphthyl)(naphthobenzofuranyl)anthracene-d₂₄; (terphenylyl)(naphthobenzofuranyl)anthracene-d₃₀; (biphenylyl)(naphthobenzofuranyl)anthracene-d₂₆; (phenylnaphthyl)(naphthobenzofuranyl)anthracene-d₂₈; (naphthyl)(naphthobenzofuranyl)anthracene-d₂₄; (terphenylyl)(naphthobenzofuranyl)anthracene-d₃₀; (biphenylyl)(naphthobenzofuranyl)anthracene-d₂₆; (binaphthyl)(naphthobenzofuranyl)anthracene-d₃₀; (phenylnaphthyl)(naphthobenzofuranyl)anthracene-d₂₈; (phenanthrenylphenyl)(naphthobenzofuranyl)anthracene-d₃₀; (naphthylphenyl)(naphthobenzofuranyl)anthracene-d₂₈; (phenanthrenylphenyl)(naphthobenzofuranyl)anthracene-d₃₀; (naphthylphenyl)(naphthobenzofuranyl)anthracene-d₂₈; (phenanthrenylphenyl)(naphthobenzofuranyl)anthracene-d₃₀; (phenanthrenylphenyl)(naphthobenzofuranyl)anthracene-d₃₀; (phenanthrenylphenyl)(naphthobenzofuranyl)anthracene-d₃₀; (naphthylphenyl)(naphthobenzofuranyl)anthracene-d₂₈; (phenanthrenyl)(naphthobenzofuranyl)anthracene-d₂₆; (phenanthrenylphenyl)(naphthobenzofuranyl)anthracene-d₃₀; (phenanthrenylphenyl)(naphthobenzofuranyl)anthracene-d₃₀; (phenanthrenylphenyl)(naphthobenzofuranyl)anthracene-d₃₀; (phenanthrenyl)(naphthobenzofuranyl)anthracene-d₂₆; (phenanthrenylphenyl)(naphthobenzofuranyl)anthracene-d₃₀; (naphthylphenyl)(naphthobenzofuranyl)anthracene-d₂₈; (phenanthrenylphenyl)(naphthobenzofuranyl)anthracene-d₃₀; naphthyl(naphthobenzofuranyl)anthracene-d₈; phenyl(naphthobenzofuranyl)anthracene-d₂; phenyl(naphthobenzofuranyl)anthracene-d₁₃; phenyl(naphthobenzofuranyl)anthracene-d₁; biphenyl(naphthobenzofuranyl)anthracene-d₈; phenyl(naphthobenzofuranyl)anthracene-d₂; phenyl(naphthobenzofuranyl)anthracene-d₈; phenyl(naphthobenzofuranyl)anthracene-d₁; phenyl(naphthobenzofuranyl)anthracene-d₂; biphenyl(naphthobenzofuranyl)anthracene-d₈; phenyl(naphthobenzofuranyl)anthracene-d₁; phenyl(naphthobenzofuranyl)anthracene-d₂; biphenyl(naphthobenzofuranyl)anthracene-d₈; phenyl(naphthobenzofuranyl)anthracene-d₁; phenyl(naphthobenzofuranyl)anthracene-d₄; phenyl[(naphthobenzofuranyl)phenyl]anthracene-d₈; biphenyl[(naphthobenzofuranyl)phenyl]anthracene-d₃₀; naphthyl[(naphthobenzofuranyl)phenyl]anthracene-d₂₈; terphenyl(naphthobenzofuranyl)anthracene-d₈; biphenyl[(naphthobenzofuranyl)phenyl]anthracene-d₈; phenyl[(naphthobenzofuranyl)phenyl]anthracene-d₈; biphenyl[(naphthobenzofuranyl)phenyl]anthracene-d₃₀; naphthyl[(naphthobenzofuranyl)phenyl]anthracene-d₂₈; (naphthylphenyl)(naphthobenzofuranyl)anthracene-d₈; phenyl[(naphthobenzofuranyl)phenyl]anthracene-d₈; phenanthrenyl[(naphthobenzofuranyl)phenyl]anthracene-d₃₀; phenanthrenyl[(naphthobenzofuranyl)phenyl]anthracene-d₃₀; terphenyl(naphthobenzofuranyl)anthracene-d₈; phenyl(naphthobenzofuranyl)anthracene; biphenyl(naphthobenzofuranyl)anthracene; biphenyl(naphthobenzofuranyl)anthracene; phenyl(phenylnaphthobenzofuranyl)anthracene; phenyl(phenylnaphthobenzofuranyl)anthracene; phenyl(phenylnaphthobenzofuranyl)anthracene; phenyl(phenylnaphthobenzofuranyl)anthracene; phenyl[(naphthobenzofuranyl)phenyl]anthracene; phenyl[(naphthobenzofuranyl)phenyl]anthracene; biphenyl[(naphthobenzofuranyl)phenyl]anthracene; biphenyl[(naphthobenzofuranyl)phenyl]anthracene; terphenyl(naphthobenzofuranyl)anthracene; terphenyl(naphthobenzofuranyl)anthracene; terphenyl(naphthobenzofuranyl)anthracene; phenyl(phenylnaphthobenzofuranyl)anthracene; phenyl(phenylnaphthobenzofuranyl)anthracene; phenyl(phenylnaphthobenzofuranyl)anthracene; biphenyl[(naphthobenzofuranyl)phenyl]anthracene; biphenyl[(naphthobenzofuranyl)phenyl]anthracene; phenyl(phenylnaphthobenzofuranyl)anthracene; biphenyl(phenylnaphthobenzofuranyl)anthracene; biphenyl(phenylnaphthobenzofuranyl)anthracene; naphthyl(phenylnaphthobenzofuranyl)anthracene; naphthyl(phenylnaphthobenzofuranyl)anthracene; (phenylnaphthyl)(phenylnaphthobenzofuranyl)anthracene; (phenylnaphthyl)(phenylnaphthobenzofuranyl)anthracene; (phenylnaphthyl)(phenylnaphthobenzofuranyl)anthracene; (phenylnaphthyl)(phenylnaphthobenzofuranyl)anthracene; (phenylnaphthyl)(phenylnaphthobenzofuranyl)anthracene; (phenylnaphthyl)(phenylnaphthobenzofuranyl)anthracene; (phenylnaphthyl)(phenylnaphthobenzofuranyl)anthracene; naphthyl[(naphthobenzofuranyl)phenyl]anthracene; naphthyl[(naphthobenzofuranyl)phenyl]anthracene; (phenylnaphthyl)(naphthobenzofuranyl)anthracene; (phenylnaphthyl)(naphthobenzofuranyl)anthracene; (phenylnaphthyl)(naphthobenzofuranyl)anthracene; (phenylnaphthyl)(naphthobenzofuranyl)anthracene; (phenylnaphthyl)(naphthobenzofuranyl)anthracene; (phenylnaphthyl)(naphthobenzofuranyl)anthracene; naphthyl[(naphthobenzofuranyl)phenyl]anthracene; naphthyl[(naphthobenzofuranyl)phenyl]anthracene; (naphthylphenyl)(naphthobenzofuranyl)anthracene; (naphthylphenyl)(naphthobenzofuranyl)anthracene; (phenanthrenyl)(naphthobenzofuranyl)anthracene; phenyl(naphthobenzofuranyl)anthracene; biphenyl(naphthobenzofuranyl)anthracene; biphenyl(naphthobenzofuranyl)anthracene; terphenyl(naphthobenzofuranyl)anthracene; terphenyl(naphthobenzofuranyl)anthracene; phenyl(phenylnaphthobenzofuranyl)anthracene; phenyl(phenylnaphthobenzofuranyl)anthracene; phenyl(phenylnaphthobenzofuranyl)anthracene; phenyl[(naphthobenzofuranyl)phenyl]anthracene; biphenyl[(naphthobenzofuranyl)phenyl]anthracene; biphenyl[(naphthobenzofuranyl)phenyl]anthracene; biphenyl(naphthobenzofuranyl)anthracene; terphenyl(naphthobenzofuranyl)anthracene; phenyl(phenylnaphthobenzofuranyl)anthracene; phenyl(phenylnaphthobenzofuranyl)anthracene; phenyl[(naphthobenzofuranyl)phenyl]anthracene; biphenyl[(naphthobenzofuranyl)phenyl]anthracene; biphenyl[(naphthobenzofuranyl)phenyl]anthracene; biphenyl[(naphthobenzofuranyl)phenyl]anthracene; biphenyl[(naphthobenzofuranyl)phenyl]anthracene; (dibenzofuranyl)[(naphthobenzofuranyl)phenyl]anthracene; (dibenzofuranyl)[(naphthobenzofuranyl)phenyl]anthracene; (dimethylfluorenyl)[(naphthobenzofuranyl)phenyl]anthracene; [(dibenzofuranyl)phenyl](naphthobenzofuranyl)anthracene; [(dimethylfluorenyl)phenyl](naphthobenzofuranyl)anthracene; (dibenzofuranyl)(naphthobenzofuranyl)anthracene; (naphthylphenyl)(naphthobenzofuranyl)anthracene; (dibenzofuranyl)[(naphthobenzofuranyl)phenyl]anthracene; (dibenzofuranyl)[(naphthobenzofuranyl)phenyl]anthracene; [(dibenzofuranyl)phenyl][(naphthobenzofuranyl)phenyl]anthracene; [(dibenzofuranyl)phenyl][(naphthobenzofuranyl)phenyl]anthracene; (dimethylfluorenyl)(naphthobenzofuranyl)anthracene; (dimethylfluorenyl)(naphthobenzofuranyl)anthracene; (dimethylfluorenyl)(naphthobenzofuranyl)anthracene; [(dibenzofuranyl)phenyl](naphthobenzofuranyl)anthracene; [(dimethylfluorenyl)phenyl](naphthobenzofuranyl)anthracene; (dimethylfluorenyl)[(naphthobenzofuranyl)phenyl]anthracene; (dimethylfluorenyl)[(naphthobenzofuranyl)phenyl]anthracene; [(dibenzofuranyl)phenyl][(naphthobenzofuranyl)phenyl]anthracene; [(dibenzofuranyl)phenyl][(naphthobenzofuranyl)phenyl]anthracene; (dimethylfluorenyl)(naphthobenzofuranyl)anthracene; (dimethylfluorenyl)(naphthobenzofuranyl)anthracene; [(dimethylfluorenyl)phenyl](naphthobenzofuranyl)anthracene; [(dimethylfluorenyl)phenyl](naphthobenzofuranyl)anthracene; [(dibenzofuranyl)phenyl](naphthobenzofuranyl)anthracene; (dimethylfluorenyl)[(naphthobenzofuranyl)phenyl]anthracene; (dimethylfluorenyl)[(naphthobenzofuranyl)phenyl]anthracene; [(dibenzofuranyl)phenyl][(naphthobenzofuranyl)phenyl]anthracene; [(dibenzofuranyl)phenyl][(naphthobenzofuranyl)phenyl]anthracene; (dibenzofuranyl)(naphthobenzofuranyl)anthracene; (dibenzofuranyl)(naphthobenzofuranyl)anthracene; (dimethylfluorenyl)(naphthobenzofuranyl)anthracene; [(dibenzofuranyl)phenyl](naphthobenzofuranyl)anthracene; [(dimethylfluorenyl)phenyl](naphthobenzofuranyl)anthracene; (naphthylphenyl)(naphthobenzofuranyl)anthracene; (dibenzofuranyl)[(naphthobenzofuranyl)phenyl]anthracene; (dibenzofuranyl)[(naphthobenzofuranyl)phenyl]anthracene; [(dibenzofuranyl)phenyl][(naphthobenzofuranyl)phenyl]anthracene; [(dibenzofuranyl)phenyl][(naphthobenzofuranyl)phenyl]anthracene; [(carbazolyl)phenyl][(naphthobenzofuranyl)phenyl]anthracene; (benzophenanthrenyl)[(naphthobenzofuranyl)phenyl]anthracene; (phenanthrenyl)(naphthobenzofuranyl)anthracene; (phenylcarbazolyl)(naphthobenzofuranyl)anthracene; (benzanthracenyl)(naphthobenzofuranyl)anthracene; (phenanthrenyl)[(naphthobenzofuranyl)phenyl]anthracene; (phenanthrenyl)[(naphthobenzofuranyl)phenyl]anthracene; (spirobifluorenyl)[(naphthobenzofuranyl)phenyl]anthracene; (spirobifluorenyl)[(naphthobenzofuranyl)phenyl]anthracene; (benzophenanthrenyl)(naphthobenzofuranyl)anthracene; (benzotriphenylenyl)(naphthobenzofuranyl)anthracene; (triphenylenyl)(naphthobenzofuranyl)anthracene; (benzotriphenylenyl)(naphthobenzofuranyl)anthracene; (benzophenanthrenyl)(naphthobenzofuranyl)anthracene; (phenanthrenyl)(naphthobenzofuranyl)anthracene; (dibenzothiophenyl)(naphthobenzofuranyl)anthracene; (benzophenanthrenyl)[(naphthobenzofuranyl)phenyl]anthracene; (benzophenanthrenyl)[(naphthobenzofuranyl)phenyl]anthracene; (diphenylfluorenyl)[(naphthobenzofuranyl)phenyl]anthracene; (diphenylfluorenyl)[(naphthobenzofuranyl)phenyl]anthracene; (benzanthracenyl)(naphthobenzofuranyl)anthracene; (phenanthrenyl)(naphthobenzofuranyl)anthracene; (phenylcarbazolyl)(naphthobenzofuranyl)anthracene; [(carbazolyl)phenyl](naphthobenzofuranyl)anthracene; (phenanthrenyl)[(naphthobenzofuranyl)phenyl]anthracene; (phenanthrenyl)[(naphthobenzofuranyl)phenyl]anthracene; (spirobifluorenyl)[(naphthobenzofuranyl)phenyl]anthracene; (spirobifluorenyl)[(naphthobenzofuranyl)phenyl]anthracene; phenyl(naphthobenzofuranyl)anthracene-d₅; biphenyl(naphthobenzofuranyl)anthracene-d₅; biphenyl(naphthobenzofuranyl)anthracene-d₅; fluorophenyl(naphthobenzofuranyl)anthracene; cyanophenyl(naphthobenzofuranyl)anthracene; di(naphthobenzofuranyl)anthracene; (dibenzofuranyl)[(naphthobenzofuranyl)phenyl]anthracene; (naphthobenzofuranyl)[(naphthobenzofuranyl)phenyl]anthracene; bis[(naphthobenzofuranyl)phenyl]anthracene; bis[(naphthobenzofuranyl)phenyl]anthracene; [(phenylnaphthyl)phenyl](naphthobenzofuranyl)anthracene; (phenylphenanthrenyl)(naphthobenzofuranyl)anthracene; (phenylbenzocarbazolyl)(naphthobenzofuranyl)anthracene; di(naphthobenzofuranyl)anthracene; [(triphenylsilyl)phenyl](naphthobenzofuranyl)anthracene; di(naphthobenzofuranyl)anthracene; (naphthobenzofuranyl)[(naphthobenzofuranyl)phenyl]anthracene; (dimethylbenzofluorenyl)(naphthobenzofuranyl)anthracene; [(benzanthracenyl)phenyl](naphthobenzofuranyl)anthracene; bis[(naphthobenzofuranyl)phenyl]anthracene; bis[(naphthobenzofuranyl)phenyl]anthracene; phenyl(naphthobenzothiophenyl)anthracene; biphenyl(naphthobenzothiophenyl)anthracene; biphenyl(naphthobenzothiophenyl)anthracene; naphthyl(naphthobenzothiophenyl)anthracene; naphthyl(naphthobenzothiophenyl)anthracene; phenyl(phenylnaphthobenzothiophenyl)anthracene; phenyl(naphthobenzothiophenyl)anthracene; phenyl[(naphthobenzothiophenyl)phenyl]anthracene; phenyl[(naphthobenzothiophenyl)phenyl]anthracene; biphenyl[(naphthobenzothiophenyl)phenyl]anthracene; biphenyl[(naphthobenzothiophenyl)phenyl]anthracene; naphthyl[(naphthobenzothiophenyl)phenyl]anthracene; naphthyl[(naphthobenzothiophenyl)phenyl]anthracene; terphenyl(naphthobenzothiophenyl)anthracene; terphenyl(naphthobenzothiophenyl)anthracene; terphenyl(naphthobenzothiophenyl)anthracene; (phenylnaphthyl)(naphthobenzothiophenyl)anthracene; (naphthylphenyl)(naphthobenzothiophenyl)anthracene; (naphthylphenyl)(naphthobenzothiophenyl)anthracene; biphenyl[(naphthobenzothiophenyl)phenyl]anthracene; biphenyl[(naphthobenzothiophenyl)phenyl]anthracene; biphenyl[(naphthobenzothiophenyl)phenyl]anthracene; biphenyl[(naphthobenzothiophenyl)phenyl]anthracene; (phenylnaphthyl)[(naphthobenzothiophenyl)phenyl]anthracene; (phenylnaphthyl)[(naphthobenzothiophenyl)phenyl]anthracene; (fluorophenyl)(naphthobenzofuranyl)anthracene; (fluorophenyl)(naphthobenzofuranyl)anthracene; (fluorophenyl)(naphthobenzofuranyl)anthracene; (fluorobiphenylyl)(naphthobenzofuranyl)anthracene; (cyanobiphenylyl)(naphthobenzofuranyl)anthracene; phenyl(naphthobenzofuranyl)anthracene-d₂₂; naphthyl(naphthobenzofuranyl)anthracene-d₂₄; naphthyl(naphthobenzofuranyl)anthracene-d₂₄; (phenylnaphthyl)(naphthobenzofuranyl)anthracene-d₂₈; (phenylnaphthyl)(naphthobenzofuranyl)anthracene-d₂₈; (phenylnaphthyl)(naphthobenzofuranyl)anthracene-d₂₈; (phenylnaphthyl)(naphthobenzofuranyl)anthracene-d₂₈; binaphthyl(naphthobenzofuranyl)anthracene-d₃₀; binaphthyl(naphthobenzofuranyl)anthracene-d₃₀; biphenyl(naphthobenzofuranyl)anthracene-d₂₄; biphenyl(naphthobenzofuranyl)anthracene-d₂₄; biphenyl(naphthobenzofuranyl)anthracene-d₂₄; terphenyl(naphthobenzofuranyl)anthracene-d₂₈; terphenyl(naphthobenzofuranyl)anthracene-d₂₈; terphenyl(naphthobenzofuranyl)anthracene-d₂₈; terphenyl(naphthobenzofuranyl)anthracene-d₂₈; (phenylnaphthyl)(naphthobenzofuranyl)anthracene-d₂₈; (naphthylphenyl)(naphthobenzofuranyl)anthracene-d₂₈; (phenanthrenylphenyl)(naphthobenzofuranyl)anthracene-d₃₀; (phenanthrenylphenyl)(naphthobenzofuranyl)anthracene-d₃₀; (phenanthrenylphenyl)(naphthobenzofuranyl)anthracene-d₃₀; (phenanthrenylphenyl)(naphthobenzofuranyl)anthracene-d₃₀; (phenanthrenylphenyl)(naphthobenzofuranyl)anthracene-d₃₀; (naphthylphenyl)(naphthobenzofuranyl)anthracene-d₂₈; (naphthylphenyl)(naphthobenzofuranyl)anthracene-d₂₈; (phenanthrenylphenyl)(naphthobenzofuranyl)anthracene-d₃₀; (phenanthrenylphenyl)(naphthobenzofuranyl)anthracene-d₃₀; (phenanthrenylphenyl)(naphthobenzofuranyl)anthracene-d₃₀; (phenanthrenylphenyl)(naphthobenzofuranyl)anthracene-d₃₀; (phenanthrenylphenyl)(naphthobenzofuranyl)anthracene-d₃₀; (phenanthrenyl)(naphthobenzofuranyl)anthracene-d₂₆; (phenanthrenyl)(naphthobenzofuranyl)anthracene-d₂₆; phenyl(naphthobenzofuranyl)anthracene-d₁₃; naphthyl(naphthobenzofuranyl)anthracene-d₂₄; naphthyl(naphthobenzofuranyl)anthracene-d₂₄; (phenylnaphthyl)(naphthobenzofuranyl)anthracene-d₂₈; (phenylnaphthyl)(naphthobenzofuranyl)anthracene-d₂₈; (phenylnaphthyl)(naphthobenzofuranyl)anthracene-d₂₈; (phenylnaphthyl)(naphthobenzofuranyl)anthracene-d₂₈; binaphthyl(naphthobenzofuranyl)anthracene-d₃₀; binaphthyl(naphthobenzofuranyl)anthracene-d₃₀; biphenyl(naphthobenzofuranyl)anthracene-d₂₆; biphenyl(naphthobenzofuranyl)anthracene-d₂₆; biphenyl(naphthobenzofuranyl)anthracene-d₂₆; terphenyl(naphthobenzofuranyl)anthracene-d₂₈; terphenyl(naphthobenzofuranyl)anthracene-d₂₈; terphenyl(naphthobenzofuranyl)anthracene-d₂₈; terphenyl(naphthobenzofuranyl)anthracene-d₂₈; (naphthylphenyl)(naphthobenzofuranyl)anthracene-d₂₈; (naphthylphenyl)(naphthobenzofuranyl)anthracene-d₂₈; (phenanthrenylphenyl)(naphthobenzofuranyl)anthracene-d₃₀; (phenanthrenylphenyl)(naphthobenzofuranyl)anthracene-d₃₀; (phenanthrenylphenyl)(naphthobenzofuranyl)anthracene-d₃₀; (phenanthrenylphenyl)(naphthobenzofuranyl)anthracene-d₃₀; (phenanthrenylphenyl)(naphthobenzofuranyl)anthracene-d₃₀; (naphthylphenyl)(naphthobenzofuranyl)anthracene-d₂₈; (naphthylphenyl)(naphthobenzofuranyl)anthracene-d₂₈; (phenanthrenylphenyl)(naphthobenzofuranyl)anthracene-d₃₀; (phenanthrenylphenyl)(naphthobenzofuranyl)anthracene-d₃₀; (phenanthrenylphenyl)(naphthobenzofuranyl)anthracene-d₃₀; (phenanthrenylphenyl)(naphthobenzofuranyl)anthracene-d₃₀; (phenanthrenylphenyl)(naphthobenzofuranyl)anthracene-d₃₀; phenanthrenyl(naphthobenzofuranyl)anthracene-d₂₆; phenanthrenyl(naphthobenzofuranyl)anthracene-d₂₆; phenyl(naphthobenzofuranyl)anthracene-d₁₃; phenyl(naphthobenzofuranyl)anthracene-d₈; phenyl(naphthobenzofuranyl)anthracene-d₂; phenyl(naphthobenzofuranyl)anthracene-d₂; phenyl(naphthobenzofuranyl)anthracene-d₄; phenyl(naphthobenzofuranyl)anthracene-d₂; phenyl(naphthobenzofuranyl)anthracene-d₁; phenyl(naphthobenzofuranyl)anthracene-d₂; phenyl(naphthobenzofuranyl)anthracene-d₁; phenyl(naphthobenzofuranyl)anthracene-d₁; phenyl(naphthobenzofuranyl)anthracene-d₁; naphthyl(naphthobenzofuranyl)anthracene-d₉; biphenyl(naphthobenzofuranyl)anthracene-d₈; biphenyl(naphthobenzofuranyl)anthracene-d₈; biphenyl(naphthobenzofuranyl)anthracene-d₈; terphenyl(naphthobenzofuranyl)anthracene-d₈; terphenyl(naphthobenzofuranyl)anthracene-d₈; (naphthylphenyl)(naphthobenzofuranyl)anthracene-d₈; terphenyl(naphthobenzofuranyl)anthracene-d₈; phenyl[(naphthobenzofuranyl)phenyl]anthracene-d₂₆; naphthyl[(naphthobenzofuranyl)phenyl]anthracene-d₂₈; naphthyl[(naphthobenzofuranyl)phenyl]anthracene-d₂₈; phenanthrenyl[(naphthobenzofuranyl)phenyl]anthracene-d₃₀; biphenyl[(naphthobenzofuranyl)phenyl]anthracene-d₃₀; biphenyl[(naphthobenzofuranyl)phenyl]anthracene-d₃₀; biphenyl[(naphthobenzofuranyl)phenyl]anthracene-d₃₀; phenanthrenyl[(naphthobenzofuranyl)phenyl]anthracene-d₃₀; phenyl[(naphthobenzofuranyl)phenyl]anthracene-d₈; phenyl[(naphthobenzofuranyl)phenyl]anthracene-d₈; biphenyl[(naphthobenzofuranyl)phenyl]anthracene-d₈; phenyl[(naphthobenzofuranyl)phenyl]anthracene-d₁₃; phenyl[(naphthobenzofuranyl)phenyl]anthracene-d₁₃; phenyl(naphthobenzofuranyl)anthracene-d₅; phenyl[(naphthobenzofuranyl)phenyl]anthracene-d₅; phenyl[(naphthobenzofuranyl)phenyl]anthracene-d₅; phenyl[(naphthobenzofuranyl)phenyl]anthracene-d₅; phenyl[(naphthobenzofuranyl)naphthyl]anthracene-d₅; phenyl[(naphthobenzofuranyl)naphthyl]anthracene-d₅; phenyl[(naphthobenzofuranyl) naphthyl]anthracene-d₅; phenyl[(naphthobenzofuranyl)naphthyl]anthracene-d₅; phenyl[(naphthobenzofuranyl)naphthyl]anthracene-d₅; phenyl(naphthobenzothiophenyl)anthracene-d₅; phenyl[(naphthobenzothiophenyl)phenyl]anthracene-d₅; phenyl[(naphthobenzothiophenyl)phenyl]anthracene-d₅; phenyl[(naphthobenzothiophenyl)phenyl]anthracene-d₅; phenyl[(naphthobenzothiophenyl)naphthyl]anthracene-d₅; phenyl[(naphthobenzothiophenyl)naphthyl]anthracene-d₆; phenyl[(naphthobenzothiophenyl)naphthyl]anthracene-d₅; phenyl[(naphthobenzothiophenyl)naphthyl]anthracene-d₆; phenyl[(naphthobenzothiophenyl)naphthyl]anthracene-d₅

### (Group 35A)

diphenylanthracene; phenyl(naphthyl)anthracene; phenyl(phenanthrenyl)anthracene; dinaphthylanthracene; dinaphthylanthracene; naphthyl(phenanthrenyl)anthracene; dinaphthylanthracene; naphthyl(phenanthrenyl)anthracene; phenyl(phenanthrenyl)anthracene; phenyl(naphthyl)anthracene; diphenanthrenylanthracene; phenyl(benzanthracenyl)anthracene; naphthyl(benzanthracenyl)anthracene; naphthyl(benzotriphenylenyl)anthracene; phenyl(benzophenanthrenyl)anthracene; naphthyl(benzanthracenyl)anthracene; phenyl(benzotriphenylenyl)anthracene; naphthyl(benzophenanthrenyl)anthracene; phenyl(naphthylphenyl)anthracene; phenyl(biphenylyl)anthracene; phenyl(naphthylphenyl)anthracene; phenyl(biphenylyl)anthracene; phenyl(naphthylphenyl)anthracene; naphthyl(biphenylyl)anthracene; naphthyl(biphenylyl)anthracene; phenyl(phenylnaphthyl)anthracene; phenyl(biphenylyl)anthracene; phenyl(naphthylphenyl)anthracene; phenyl(phenylnaphthyl)anthracene; naphthyl(naphthylphenyl)anthracene; naphthyl(biphenylyl)anthracene; dinaphthylanthracene; naphthyl(naphthylphenyl)anthracene; naphthyl(naphthylphenyl)anthracene; naphthyl(phenylnaphthyl)anthracene; naphthyl(naphthylphenyl)anthracene; naphthyl(phenylnaphthyl)anthracene; naphthyl(naphthylphenyl)anthracene; naphthyl(biphenylyl)anthracene; naphthyl(naphthylphenyl)anthracene; naphthyl(biphenylyl)anthracene; naphthyl(biphenylyl)anthracene; naphthyl(naphthylphenyl)anthracene; naphthyl(naphthylphenyl)anthracene; phenanthrenyl(biphenylyl)anthracene; phenanthrenyl(naphthylphenyl)anthracene; phenanthrenyl(biphenylyl)anthracene; phenanthrenyl(naphthylphenyl)anthracene; naphthyl(naphthylphenyl)anthracene; (phenylnaphthyl)(naphthylphenyl)anthracene; (phenylnaphthyl)(biphenylyl)anthracene; (phenylnaphthyl)(naphthylphenyl)anthracene; (phenylnaphthyl)(biphenylyl)anthracene; (phenylnaphthyl)(naphthylphenyl)anthracene; (phenylnaphthyl)(naphthylphenyl)anthracene; (phenylnaphthyl)(naphthylphenyl)anthracene; benzophenanthrenyl(phenylnaphthyl)anthracene; (benzophenanthrenylphenyl)(biphenylyl)anthracene; benzophenanthrenyl(biphenylyl)anthracene; naphthyl(benzophenanthrenylphenyl)anthracene; benzophenanthrenyl(naphthylphenyl)anthracene; benzophenanthrenyl(biphenylyl)anthracene; (benzophenanthrenylphenyl)(biphenylyl)anthracene; benzophenanthrenyl(phenylnaphthyl)anthracene; phenyl(benzophenanthrenylphenyl)anthracene; benzophenanthrenyl(biphenylyl)anthracene; naphthyl(benzophenanthrenylphenyl)anthracene; benzotriphenylenyl(biphenylyl)anthracene; benzotriphenylenyl(phenylnaphthyl)anthracene; (benzotriphenylenylphenyl)(naphthylphenyl)anthracene; benzophenanthrenyl(naphthylphenyl)anthracene; benzotriphenylenyl(naphthylphenyl)anthracene; benzotriphenylenyl(phenanthrenyl)anthracene; benzotriphenylenyl(naphthylphenyl)anthracene; benzotriphenylenyl(naphthylphenyl)anthracene; benzotriphenylenyl(biphenylyl)anthracene; benzotriphenylenyl(naphthylphenyl)anthracene; benzotriphenylenyl(biphenylyl)anthracene; benzanthracenyl(naphthylphenyl)anthracene; benzanthracenyl(biphenylyl)anthracene; naphthyl(phenanthrenyl)anthracene-d₂₄; phenyl(naphthyl)anthracene-d₂₀; dinaphthylanthracene-d₂₂; naphthyl(biphenylyl)anthracene-d₂₄; naphthyl(phenanthrenyl)anthracene-d₂₄; naphthyl(biphenylyl)anthracene-d₂₄; naphthyl(phenanthrenyl)anthracene-d₂₄; naphthyl(phenanthrenyl)anthracene-d₂₄; naphthyl(phenanthrenyl)anthracene-d₂₄; naphthyl(biphenylyl)anthracene-d₂₄; naphthyl(phenanthrenylphenyl)anthracene-d₂₈; naphthyl(phenanthrenylphenyl)anthracene-d₂₈; naphthyl(phenanthrenylphenyl)anthracene-d₂₈; naphthyl(terphenylyl)anthracene-d₂₈; naphthyl(naphthylphenyl)anthracene-d₂₆; naphthyl(phenanthrenylphenyl)anthracene-d₂₈; naphthyl(terphenylyl)anthracene-d₂₈; naphthyl(terphenylyl)anthracene-d₂₈; naphthyl(naphthylphenyl)anthracene-d₂₆; naphthyl(naphthylphenyl)anthracene-d₂₆; naphthyl(phenanthrenylphenyl)anthracene-d₂₈; naphthyl(terphenylyl)anthracene-d₂₈; naphthyl(naphthylphenyl)anthracene-d₂₆; naphthyl(phenanthrenylphenyl)anthracene-d₂₈; naphthyl(binaphthyl)anthracene-d₂₈; naphthyl(phenylnaphthyl)anthracene-d₂₆; naphthyl(phenylnaphthyl)anthracene-d₂₆; naphthyl(binaphthyl)anthracene-d₂₈; naphthyl(phenanthrenyl)anthracene-d₂₄; phenyl(naphthyl)anthracene-d₂₀; dinaphthylanthracene-d₂₂; dinaphthylanthracene-d₂₂; naphthyl(biphenylyl)anthracene-d₂₄; naphthyl(phenanthrenyl)anthracene-d₂₄; naphthyl(biphenylyl)anthracene-d₂₄; naphthyl(phenanthrenyl)anthracene-d₂₄; naphthyl(phenanthrenyl)anthracene-d₂₄; naphthyl(biphenylyl)anthracene-d₂₄; naphthyl(phenanthrenylphenyl)anthracene-d₂₈; naphthyl(terphenylyl)anthracene-d₂₈; naphthyl(naphthylphenyl)anthracene-d₂₆; naphthyl(phenanthrenylphenyl)anthracene-d₂₈; naphthyl(terphenylyl)anthracene-d₂₈; naphthyl(terphenylyl)anthracene-d₂₈; naphthyl(naphthylphenyl)anthracene-d₂₆; naphthyl(naphthylphenyl)anthracene-d₂₆; naphthyl(phenanthrenylphenyl)anthracene-d₂₈; naphthyl(terphenylyl)anthracene-d₂₈; naphthyl(naphthylphenyl)anthracene-d₂₆; naphthyl(phenanthrenylphenyl)anthracene-d₂₈; phenanthrenyl(phenanthrenylphenyl)anthracene-d₃₀; phenyl(phenanthrenyl)anthracene-d₂₂; phenanthrenyl(naphthylphenyl)anthracene-d₂₈; diphenanthrenylanthracene-d₂₆; phenanthrenyl(biphenylyl)anthracene-d₂₆; diphenanthrenylanthracene-d₂₆; phenanthrenyl(naphthylphenyl)anthracene-d₂₈; phenanthrenyl(phenanthrenylphenyl)anthracene-d₃₀; phenyl(phenanthrenyl)anthracene-d₂₂; phenanthrenyl(naphthylphenyl)anthracene-d₂₈; diphenanthrenylanthracene-d₂₆; phenanthrenyl(biphenylyl)anthracene-d₂₆; diphenylanthracene-d₁₈; bis(naphthylphenyl)anthracene-d₃₀; phenyl(naphthylphenyl)anthracene-d₂₄; phenyl(phenylnaphthyl)anthracene-d₂₄; naphthyl(phenylphenyl)anthracene-d₂₆; phenyl(phenylnaphthyl)anthracene-d₂₄; naphthyl(naphthylphenyl)anthracene-d₈; phenyl(naphthylphenyl)anthracene-d₈; naphthyl(naphthylphenyl)anthracene-d₈; naphthyl(phenanthrenylphenyl)anthracene-d₈; phenyl(phenanthrenylphenyl)anthracene-d₈; naphthyl(phenanthrenylphenyl)anthracene-d₈; naphthyl(biphenylyl)anthracene-d₈; phenyl(biphenylyl)anthracene-d₈; naphthyl(biphenylyl)anthracene-d₈; naphthyl(phenanthrenylphenyl)anthracene-d₈; phenyl(phenanthrenylphenyl)anthracene-d₈; naphthyl(phenanthrenylphenyl)anthracene-d₈; naphthyl(phenanthrenyl)anthracene-d₈; phenyl(phenanthrenyl)anthracene-d₈; naphthyl(phenanthrenyl)anthracene-d₈; naphthyl(biphenylyl)anthracene-d₈; phenyl(biphenylyl)anthracene-d₈; naphthyl(biphenylyl)anthracene-d₈; naphthyl(naphthylphenyl)anthracene-d₈; naphthyl(naphthylphenyl)anthracene-d₈; naphthyl(naphthylphenyl)anthracene-d₈; naphthyl(phenyl)anthracene-d₈; diphenylanthracene-d₈; naphthyl(phenyl)anthracene-d₈; phenyl(phenylnaphthyl)anthracene-d₈; naphthyl(naphthylphenyl)anthracene-d₈; naphthyl(naphthylphenyl)anthracene-d₈; naphthyl(phenanthrenylphenyl)anthracene-d₈; phenyl(phenylnaphthyl)anthracene-d₈; phenyl(phenylnaphthyl)anthracene-d₈; naphthyl(naphthylphenyl)anthracene-d₈; dinaphthylanthracene-d₁₅; naphthyl(biphenylyl)anthracene-d₁₅; naphthyl(phenanthrenyl)anthracene-d₁₅; naphthyl(naphthylphenyl)anthracene-d₁₅; naphthyl(naphthylphenyl)anthracene-d₁₅; naphthyl(naphthylphenyl)anthracene-d₁₅; naphthyl(phenanthrenylphenyl)anthracene-d₁₅; naphthyl(naphthylphenyl)anthracene-d₁₅; naphthyl(naphthylphenyl)anthracene-d₁₉; naphthyl(naphthylphenyl)anthracene-d₁₉; naphthyl(naphthylphenyl)anthracene-d₁₉; naphthyl(naphthylphenyl)anthracene-d₁₉; naphthyl(naphthylphenyl)anthracene-d₁₇; naphthyl(naphthylphenyl)anthracene-d₁₇; naphthyl(phenanthrenylphenyl)anthracene-d₁₇; naphthyl(naphthylphenyl)anthracene-d₁₉; naphthyl(naphthylphenyl)anthracene-d₁₉; dinaphthylanthracene-d₁₄; naphthyl(biphenylyl)anthracene-d₄; dinaphthylanthracene-d₁₄; naphthyl(biphenylyl)anthracene-d₄; phenyl(triphenylenyl)anthracene-d₈; phenyl(benzophenanthrenyl)anthracene-d₈; phenyl(benzanthracenyl)anthracene-d₈; phenyl(benzotriphenylenyl)anthracene-d₈; triphenylenyl(naphthylphenyl)anthracene-d₈; benzophenanthrenyl(naphthylphenyl)anthracene-d₈; benzanthracenyl(naphthylphenyl)anthracene-d₈; benzotriphenylenyl(naphthylphenyl)anthracene-d₈; triphenylenyl(naphthylphenyl)anthracene-d₈; benzophenanthrenyl(naphthylphenyl)anthracene-d₈; benzanthracenyl(naphthylphenyl)anthracene-d₈; benzotriphenylenyl(naphthylphenyl)anthracene-d₈; triphenylenyl(biphenylyl)anthracene-d₈; benzophenanthrenyl(biphenylyl)anthracene-d₈; benzanthracenyl(biphenylyl)anthracene-d₈; benzotriphenylenyl(biphenylyl)anthracene-d₈; naphthyl(benzophenanthrenyl)anthracene-d₂₆; naphthyl(benzophenanthrenyl)anthracene-d₁₅; phenyl(benzophenanthrenyl)anthracene-d₂₄; phenyl(benzophenanthrenyl)anthracene-d₁₃
In one embodiment, as the first component and the second component, one or more compounds selected from compounds corresponding to each of the first component and the second component in compounds included in each group of the following (Group 1B) to (Group 35B) can be independently used. The first component and the second component in the present specification are not particularly limited independently to one or more compounds selected from compounds corresponding to each of the first component and the second component in compounds included in each group of the following (Group 1B) to (Group 35B), and they may not be these.

### (Group 1B)

9,10-di(2-naphthyl)anthracene)(ADN); 2-tert-butyl-9,10-di(2-naphthyl)anthracene(TBADN); 5,6,11,12-tetraphenyltetracene(rubrene); 2,5,8,11-tetra-tert-butylperylene(TBP)

### (Group 2B)

4,4′-bis[N-phenylamino]biphenyl; 4,4′-bis[(1-naphthyl)-N-phenylamino]biphenyl(NBP); 5,6,11,12-tetraphenyltetracene(rubrene); 9,10-bis[N-(1-phenylamino)biphenyl]; 2,5,8,11-tetra-tert-butylperylene(TBP); tris(8-quinolinolato-N1,O8)aluminum(Alq); coumarin545T

### (Group 3B)

N,N′-di(1-naphthyl)-N,N′-diphenyl-(1,1′-biphenyl)-4,4′-diamine(NPD); Alq3; 10-(2-benzothiazolyl)-1,1,7,7-tetramethyl-2,3,6,7-tetrahydro-1H,5H, 11H-[1]benzopyrano[6,7,8-ij]quinolizine-11-one(C545T); rubrene(5,6,11,12-tetraphenyltetracene); 4,4′-bis(2,2-diphenylvinyl)biphenyl(DPVBi)

### (Group 4B)

### TBADN; TBP

### (Group 5B)

TBADN; Alq3; TBP; 5,6,11,12-tetraphenyltetracene(rubrene); 4-(dicyanomethylene)-2-tert-butyl-6-(1,1,7,7-tetramethyljulolidine-4-yl-vinyl)-4H-pyran(DCJTB)

### (Group 6B)

tetracene; tetrafluoro-tetracyano-quinodimethane(F4-TCNQ); 4,4′,4″-tris[phenyl(m-tolyl)amino]triphenylamine; naphthalenetetracarboxylic anhydride(NTCDA); leuco crystal violet(LCV); N,N′-bis(3-methylphenyl)-N,N′-bis(phenyl)-benzidine(TPD)

### (Group 9B)

1-(2,2′-binaphthalene-6-yl)-3-(phenanthrene-9-yl)benzene; iridium(III)bis(2-phenylquinolyl-N,C²′)acetylacetonate; 9-{3-[6-phenyl-2-phenylpyrimidine-4-yl]-5-(9H-carbazole-9-yl)phenyl}-9H-carbazole; tris[2-phenylpyridinate-C₂, N]iridium(III)

### (Group 10B)

1,3-di(9H-carbazole-9-yl)benzene; [2,2′-bis(2′,4′-difluoro-2,3′-bipyridinato-2-yl)propane-C,N,N,C]platinum(II); 4,4′,4″-tris(N,N-(2-naphthyl)-phenylamino)triphenylamine(2-TNATA); 2,3,5,6-tetrafluoro-7,7,8,8-tetracyano-quinodimethane(F4-TCNQ); 3,3′-di(9H-carbazole-9-yl)-1,1′-biphenyl; [2-(2′,4′-difluoro-2,3′-bipyridinato-2-yl)-2-{6-(5-trifluoromethyl-1H-pyrazole-1-yl)pyridinato-2-yl-C,N,N,C}propane-C,N,N,C]platinum(II); [2,2′-bis(2,3′-bipyridinato-2-yl)propane-C,N,N,C]platinum(II); iridium(III)bis(2-phenylquinolyl-N,C²′)acetylacetonate; 9,10-di(2-naphthyl)anthracene; 2,5,8,11-tetra-tert-butylperylene; 10-(2-benzothiazolyl)-1,1,7,7-tetramethyl-11-oxo-1,2,3,5,6,7-hexahydro-11H-[1]benzopyrano[6,7,8-ij]quinolizine-9-id; 2-[6-[2-[(2,3,6,7-tetrahydro-1H,5H-benzo[ij]quinolizine)-9-yl]ethenyl]-2-methyl-4H-pyran-4-ylidene]propanedinitrile

### (Group 12B)

2-{4′-triphenylenyl-[1,1′-biphenyl]-3-yl}-4,6-diphenyl-1,3,5-triazine; 9-[4,6-diphenyl-2-pyridyl]-9′-phenyl-3,3′-bis[9H-carbazole]; 9-(1,1′-biphenyl-3-yl)-9′-(1,1′-biphenyl-4-yl)-3,3′-bis[9H-carbazole]

### (Group 13B)

9-[3′-(triphenylene-2-yl)-1,1′-biphenyl]-9H-carbazole; 11-(4,6-diphenyl-1,3,5-triazine-2-yl)-12-(4′-phenyl-1,1′-biphenyl-4-yl)-11H, 12H-indolo[2,3-a]carbazole

### (Group 14B)

2-(1-(1,1′-biphenyl-4-yl)-9H-carbazole-9-yl)-4,6-diphenyl-1,3,5-triazine; iridium(III)bis(2-(3,5-dimethylphenyl)-5-(2-methylpropyl)quinolyl-N,C²′)acetylacetonate; 2-(1-(9,9′-dimethylfluorene-2-yl)-9H-carbazole-9-yl)-4,6-diphenyl-1,3,5-triazine; iridium(III)bis(2-(3,5-dimethylphenyl)quinolyl-N,C²′)acetylacetonate; 2-(1-phenyl-9H-carbazole-9-yl)-4-(9,9′-dimethylfluorene-2-yl)-6-phenyl-1,3,5-triazine; iridium(III)bis(2-(3,5-dimethylphenyl)quinolyl-N,C²′)2,2,8,8-tetramethyl-4,6-nonanedionate; 2-(1-(1,1′-biphenyl-4-yl)-9H-carbazole-9-yl)-4,6-diphenyl-1,3,5-triazine; iridium(III)bis(2-(3,5-dimethylphenyl)-7-(2-methylpropyl)quinolyl-N,C²′)acetylacetonate; 2-(1-phenyl-9H-carbazole-9-yl)-4-(9,9′-dimethylfluorene-2-yl)-6-phenyl-1,3,5-triazine; iridium(III)bis(2-(3,5-dimethylphenyl)-5-cyclopentylquinolyl-N,C²′)acetylacetonate; 2-(1-(1,1′-biphenyl-3-yl)-9H-carbazole-9-yl)-4,6-diphenyl-1,3,5-triazine; iridium(III)bis(2-(3,5-dimethylphenyl)-6-(2-methylpropyl)quinolyl-N,C²′)acetylacetonate; 11-(1,1′-biphenyl-4-yl)-12-(4,6-diphenyl-1,3,5-triazine-2-yl)-11H, 12H-indolo[2,3-a]carbazole; iridium(III)bis(2-phenylpyridyl-N,C²′)(4-methyl-5-(4-fluorophenyl)-2-phenylpyridyl-N, C₂′); 11-phenyl-12-(4-(1,1′-biphenyl-4-yl)-6-phenyl-1,3,5-triazine-2-yl)-11H, 12H-indolo[2,3-a]carbazole; iridium(III)bis(2-phenylpyridyl-N,C²′)(2,5-diphenylpyridyl-N,C₂′); 12-(4,6-diphenyl-1,3,5-triazine-2-yl)-12H-[1]benzothieno[2,3-a]carbazole; iridium(III)bis(2-phenylpyridyl-N,C²′)(4-isopropyl-2,5-diphenylpyridyl-N,C₂′); 11-phenyl-12-(4-(9,9-dimethylfluorene-2-yl)-6-phenyl-1,3,5-triazine-2-yl)-11H, 12H-indolo[2,3-a]carbazole; iridium(III)bis(2-phenylpyridyl-N,C²′)(5-(4-fluorophenyl)-2-phenylpyridyl-N,C²′); 5-phenyl-11-(4,6-diphenyl-1,3,5-triazine-2-yl)-5H, 11H-indolo[3,2-b]carbazole; iridium(III)bis(2-phenylpyridyl-N,C²′)(4-methyl-2,5-diphenylpyridyl-N,C₂′); iridium(III)bis(2-phenylpyridyl-N,C²′)(4-(methyl-d₃)-2,5-diphenylpyridyl-N,C₂′); 5-phenyl-11-(4-(9,9-dimethylfluorene-2-yl)-6-phenyl-1,3,5-triazine-2-yl)-5H, 11H-indolo[3,2-b]carbazole; iridium(III)bis(2-phenylpyridyl-N,C²′)(4-(4-methylphenyl)-2-phenylpyridyl-N,C₂′); 2-(7,7-dimethylindeno[2,1-b]carbazole-5-yl)-4-(1,1′-biphenyl-4-yl)-6-phenyl-1,3,5-triazine; iridium(III)bis(2-phenylpyridyl-N,C²′)(4-(4-fluorophenyl)-2-phenylpyridyl-N, C₂′); 5-phenyl-12-(4,6-diphenyl-1,3,5-triazine-2-yl)-5H, 12H-indolo[3,2-a]carbazole; iridium(III)bis(5-methyl-2-phenylpyridyl-N,C²′)(4-methyl-2,5-diphenylpyridyl-N,C₂′); iridium(III)bis(5-(methyl-d₃)-2-phenylpyridyl-N,C²′)(4-(methyl-d₃)-5-phenyl-2-phenylpyridyl-N,C₂′)

### (Group 15B)

4-[3-(triphenylene-2-yl)phenyl]-6-phenyldibenzothiophene; 2-(6-(1,1′-biphenyl-4-yl)-dibenzothiophene4-yl)-4,6-diphenyl-1,3,5-triazine; iridium(III)bis(2-phenylpyridyl-N,C²′)(4-methyl-2,5-diphenylpyridyl-N,C₂′); 2-(6-(4′-phenyl-1,1′-biphenyl-3-yl)-dibenzothiophene4-yl)-4,6-diphenyl-1,3,5-triazine; 2-{3-(6-(4′-phenyl-1,1′-biphenyl-3-yl)-dibenzothiophene4-yl)phenyl}-4,6-diphenyl-1,3,5-triazine; 2-{3-(6-(1,1′-biphenyl-3-yl)-dibenzothiophene4-yl)phenyl}-4,6-diphenyl-1,3,5-triazine; 2-{3-(dibenzothiophene4-yl)phenyl}-4,6-diphenyl-1,3,5-triazine; 2-{3-(dibenzothiophene4-yl)-5-(1,1′-biphenyl-4-yl)-phenyl}-4,6-diphenyl-1,3,5-triazine; 2-{3-(dibenzothiophene4-yl)-5-(9,9-dimethylfluorene-2-yl)-phenyl}-4,6-diphenyl-1,3,5-triazine; 2-{3-(6-(1,1′-biphenyl-4-yl)-dibenzothiophene4-yl)phenyl}-4,6-diphenyl-1,3,5-triazine; 2,4,6-tri(1,1′-biphenyl-4-yl)-1,3,5-triazine; 2,4-bis(terphenyl4-yl)-6-phenyl-1,3,5-triazine; 2-(3-(3-pyridyl)-5-(phenanthrene-9-yl)-phenyl)-4,6-diphenyl-1,3,5-triazine; 2-(9,9-dimethylfluorene-2-yl)-4,6-bis(1,1′-biphenyl-3-yl)-1,3,5-triazine; 2-(3-(dibenzothiophene4-yl)-phenyl)-4,6-bis(1,1′-biphenyl-3-yl)-1,3,5-triazine; 2-(3-(9-(2-pyridyl)-9H-carbazole-3-yl)-5-(dibenzothiophene4-yl)-phenyl)-4,6-diphenyl-1,3,5-triazine; 2-(9H-carbazole-9-yl)-4-phenyl-6-(triphenylene-2-yl)-1,3,5-triazine; 2-{5-(6-phenyl-dibenzofuran-4-yl)-1,1′-biphenyl3-yl}-4,6-diphenyl-1,3,5-triazine; 2-{5-(6-phenyldibenzothiophene4-yl)-1,1′-biphenyl-3-yl}-4,6-diphenyl-1,3,5-triazine; 2-{5-(6-(1,1′-biphenyl-4-yl)-dibenzothiophene4-yl)-1,1′-biphenyl-3-yl}-4,6-diphenyl-1,3,5-triazine; 2-{5-(6-(1,1′-biphenyl-3-yl)-dibenzothiophene4-yl)-1,1′-biphenyl3-yl}-4,6-diphenyl-1,3,5-triazine; 2-(5-(dibenzothiophene4-yl)-1,1′-biphenyl-3-yl)-4-(1,1′-biphenyl-4-yl)-6-phenyl-1,3,5-triazine; 2-(5-(dibenzothiophene4-yl)-1,1′-biphenyl-3-yl)-4-(9,9-dimethylfluorene-2-yl)-6-phenyl-1,3,5-triazine; 2-(5-(6-phenyldibenzothiophene4-yl)-1,1′-biphenyl-3-yl)-4-(9,9-dimethylfluorene-2-yl)-6-phenyl-1,3,5-triazine; 2-{3′-phenyl-5′-(dibenzothiophene4-yl)-1,1′-biphenyl-3-yl}-4,6-diphenyl-1,3,5-triazine; 2-{5-phenyl-5′-(dibenzothiophene4-yl)-1,1′-biphenyl-3-yl}-4,6-diphenyl-1,3,5-triazine; 2-{5-(9,9-dimethylfluorene-2-yl)-4′-phenyl-1,1′-biphenyl-3-yl}-4,6-diphenyl-1,3,5-triazine; 2-{3,5-bis(9,9-dimethylfluorene-2-yl)-phenyl}-4,6-diphenyl-1,3,5-triazine; 2-(3-(6-phenyldibenzothiophene4-yl)-phenyl)-4-(1,1′-biphenyl-3-yl)-6-phenyl-1,3,5-triazine; 2-{4-(6-phenyldibenzothiophene4-yl)-phenyl}-4,6-diphenyl-1,3,5-triazine; 2-{3′-(6-phenyldibenzothiophene4-yl)-1,1′-biphenyl-4-yl}-4,6-diphenyl-1,3,5-triazine; 2-{4′-(6-phenyldibenzothiophene4-yl)-1,1′-biphenyl-3-yl}-4,6-diphenyl-1,3,5-triazine; 2-(6-(1,1′-biphenyl-4-yl)-dibenzothiophene4-yl)-4-(1,1′-biphenyl-3-yl)-6-phenyl-1,3,5-triazine; 2-{4-(6-(1,1′-biphenyl-4-yl)-dibenzothiophene4-yl)-phenyl}-4,6-diphenyl-1,3,5-triazine; 2-{4-(6-(1,1′-biphenyl-3-yl)-dibenzothiophene4-yl)-phenyl}-4,6-diphenyl-1,3,5-triazine; 2-(6-phenyldibenzothiophene4-yl)-4-(9,9-dimethylfluorene-2-yl)-6-phenyl-1,3,5-triazine; 2-{(6-(1,1′-biphenyl-4-yl)-dibenzothiophene4-yl}-4-(9,9-dimethylfluorene-2-yl)-6-phenyl-1,3,5-triazine; 2-{3-(6-(1,1′-biphenyl-4-yl)-dibenzothiophene4-yl)-phenyl}-4-(9,9-dimethylfluorene-2-yl)-6-phenyl-1,3,5-triazine; 2-{3-(2,7-diphenyldibenzothiophene4-yl)-phenyl}-4,6-diphenyl-1,3,5-triazine; 2-{4′-(6-(1,1′-biphenyl-4-yl)-dibenzothiophene4-yl)-1,1′-biphenyl-3-yl}-4,6-diphenyl-1,3,5-triazine; 2-(9,9-dimethylfluorene-2-yl)-4,6-bis(4′-phenyl-1,1′-biphenyl-3-yl)-1,3,5-triazine; 2-(dibenzothiophene4-yl)-4-(9,9-dimethylfluorene-2-yl)-6-(4′-phenyl-1,1′-biphenyl-3-yl)-1,3,5-triazine; 2-[3′-(triphenylene-2-yl)-1,1′-biphenyl-4-yl]-4,6-diphenyl-1,3,5-triazine; 2-[4′-(dibenzofuran-4-yl)-1,1′-biphenyl-3-yl]-4,6-diphenyl-1,3,5-triazine; 4-(triphenylene-2-yl)-dibenzofuran; 4-(triphenylene-2-yl)-dibenzothiophene; 4-(3-(triphenylene-2-yl)phenyl)-dibenzothiophene; 4-(3-phenyl-5-(triphenylene-2-yl)phenyl)-dibenzothiophene; 4-(1,1′-biphenyl-4-yl)-5-(triphenylene-2-yl)-dibenzothiophene; 4-(9H-carbazole-9-yl)-5-(triphenylene-2-yl)-dibenzofuran; 4-(9H-carbazole-9-yl)-5-(triphenylene-2-yl)-dibenzothiophene; 9-(3′-(triphenylene-2-yl)-1,1′-biphenyl-3-yl)-9H-carbazole; 9-(3′-(triphenylene-2-yl)-1,1′-biphenyl-4-yl)-9H-carbazole; [2-{3-(triphenylene-2-yl)phenyl}pyridine-3-yl]-9H-carbazole; 2-(6-phenyldibenzofuran-4-yl)-4,6-diphenyl-1,3,5-triazine; 2-(6-phenyldibenzothiophene4-yl)-4,6-diphenyl-1,3,5-triazine; 2-(6-(1,1′-biphenyl-4-yl)dibenzothiophene4-yl)-4,6-diphenyl-1,3,5-triazine; 2-[3-(6-(1,1′-biphenyl-4-yl)dibenzofuran-4-yl)phenyl]-4,6-diphenyl-1,3,5-triazine; 2-(6-phenyldibenzothiophene4-yl)-4,6-bis(1,1′-biphenyl-3-yl)-1,3,5-triazine; 2-[3-(6-(1,1′-biphenyl-4-yl)dibenzofuran-4-yl)phenyl]-4,6-bis(phenyl-d₅)-1,3,5-triazine; 2-{3-(dibenzothiophene4-yl)-5-(1,1′-biphenyl-4-yl)phenyl}-4,6-diphenyl-1,3,5-triazine; 9-[3-(9-(4′-phenyl-1,1′-biphenyl-3-yl)-9H-carbazole-3-yl)phenyl]-9H-carbazole; 9-[3-(9-(4′-phenyl-1,1′-biphenyl-3-yl)-9H-carbazole-2-yl)phenyl]-9H-carbazole; 9-[3-(9-(triphenylene-2-yl)-9H-carbazole-3-yl)phenyl]-9H-carbazole; 9-[3-(9-(triphenylene-2-yl)-9H-carbazole-2-yl)phenyl]-9H-carbazole; 9-[3-(9-(dibenzothiophene4-yl)-9H-carbazole-3-yl)phenyl]-9H-carbazole; 2-(dibenzofuran-4-yl)-4-(1,1′-biphenyl-4-yl)-6-(4′-phenyl-1,1′-biphenyl-3-yl)-1,3,5-triazine; 2-(dibenzothiophene4-yl)-4-(1,1′-biphenyl-4-yl)-6-(4′-phenyl-1,1′-biphenyl-3-yl)-1,3,5-triazine; 2-(dibenzothiophene4-yl)-4-(9,9-dimethylfluorene-2-yl)-6-(4′-phenyl-1,1′-biphenyl-3-yl)-1,3,5-triazine; 2-(biphenylene-3-yl)-4-(9,9-dimethylfluorene-2-yl)-6-(4′-phenyl-1,1′-biphenyl-3-yl)-1,3,5-triazine

### (Group 16B)

11-(4-(1,1′-biphenyl-4-yl)-6-phenyl-1,3,5-triazine-2-yl)-12-phenyl-11H, 12H-indolo[2,3-a]carbazole; 5-phenyl-12-(6-(9H-carbazole-9-yl)pyridine-2-yl)-5H, 12H-indolo[3,2-a]carbazole; 5-(9-phenylcarbazole-3-yl)-12-phenyl-5H, 12H-indolo[3,2-a]carbazole; 2-(3-(triphenylene-2-yl)phenyl)-dibenzothiophene; 5,11-bis(2-phenyl-pyridinyl-6-yl)-5H, 11H-indolo[3,2-b]carbazole; 3,3′-di(9H-carbazole-9-yl)-1,1′-biphenyl

### (Group 17B)

9-(dibenzothiophene4-yl)-9′-phenyl-3,3′-bi[9H-carbazole]; 2-[3-(6-(1,1′-biphenyl-4-yl)dibenzofuran-4-yl)phenyl]-4,6-diphenyl-1,3,5-triazine; 2-[3-(6-(1,1′-biphenyl-4-yl)dibenzofuran-4-yl)phenyl]-4,6-bis(phenyl-d₅)-1,3,5-triazine

### (Group 18B)

5-(1,1′-biphenyl-4-yl)-8-(4′-phenyl-1,1′-biphenyl-4-yl)-5H,8H-indolo[2,3-c]carbazole; iridium(III)bis(2-phenylpyridyl-N,C²′)(4-methyl-2,5-diphenylpyridyl-N,C₂′); 5,8-bis(1,1′-biphenyl-4-yl)-5H,8H-indolo[2,3-c]carbazole; 5-(4′-phenyl-1,1′-biphenyl-3-yl)-12-(9,9-dimethylfluorene-2-yl)-5H, 12H-indolo[3,2-a]carbazole; 5-(1,1′-biphenyl-4-yl)-12-(9,9-dimethylfluorene-2-yl)-5H, 12H-indolo[3,2-a]carbazole; 2-{3-(dibenzothiophene4-yl)-5-(1,1′-biphenyl-4-yl)-phenyl}-4,6-diphenyl-1,3,5-triazine; 5-(1,1′-biphenyl-4-yl)-8-(4′-phenyl-1,1′-biphenyl-3-yl)-5H,8H-indolo[2,3-c]carbazole; 5-(4′-phenyl-1,1′-biphenyl-4-yl)-8-(1,1′-biphenyl-3-yl)-5H,8H-indolo[2,3-c]carbazole; 2-{5-(6-phenyldibenzothiophene4-yl)-1,1′-biphenyl-3-yl}-4,6-diphenyl-1,3,5-triazine; 5-(4′-phenyl-1,1′-biphenyl-4-yl)-12-(1,1′-biphenyl-4-yl)-5H, 12H-indolo[3,2-a]carbazole; 5-(4′-phenyl-1,1′-biphenyl-3-yl)-8-(dibenzothiophene4-yl)-5H,8H-indolo[2,3-c]carbazole; 2-{5-(6-(1,1′-biphenyl-4-yl)dibenzothiophene4-yl)-1,1′-biphenyl-3-yl}-4,6-diphenyl-1,3,5-triazine; 5-(4′-phenyl-1,1′-biphenyl-4-yl)-8-(triphenylene-2-yl)-5H,8H-indolo[2,3-c]carbazole; 5-(4′-phenyl-1,1′-biphenyl-4-yl)-8-(3-(dibenzothiophene4-yl)phenyl)-5H,8H-indolo[2,3-c]carbazole; 5-(4′-phenyl-1,1′-biphenyl-3-yl)-8-(3-(9H-carbazole-9-yl)phenyl)-5H,8H-indolo[2,3-c]carbazole; 5-(4′-phenyl-1,1′-biphenyl-4-yl)-8-(dibenzofuran-2-yl)-5H,8H-indolo[2,3-c]carbazole; 5-(4′-phenyl-1,1′-biphenyl-4-yl)-12-(4′-phenyl-1,1′-biphenyl-3-yl)-5H, 12H-indolo[3,2-a]carbazole; 2-{3-(6-(1,1′-biphenyl-4-yl)dibenzothiophene4-yl)phenyl}-4,6-diphenyl-1,3,5-triazine; 5-(4′-(phenyl-d₅)-1,1′-biphenyl-4-yl)-8-(phenyl-d₅)-5H,8H-indolo[2,3-c]carbazole; 5-(4′-phenyl-1,1′-biphenyl-3-yl)-12-phenyl-5H, 12H-indolo[3,2-a]carbazole; 2-{4-(6-(1,1′-biphenyl-4-yl)dibenzothiophene4-yl)phenyl}-4,6-diphenyl-1,3,5-triazine; 2-{3-(6-(1,1′-biphenyl-4-yl)dibenzothiophene4-yl)phenyl}-4,6-bis(phenyl-d₅)-1,3,5-triazine; 5-(3′-(phenyl-d₅)-1,1′-biphenyl-4-yl)-8-(phenyl-d₅)-5H,8H-indolo[2,3-c]carbazole; 2-{3-(6-(9,9-dimethylfluorene-2-yl)dibenzothiophene4-yl)phenyl}-4,6-diphenyl-1,3,5-triazine; 5-(4′-phenyl-1,1′-biphenyl-4-yl)-12-phenyl-5H, 12H-indolo[3,2-a]carbazole; 2-{5-(6-(9,9-dimethylfluorene-2-yl)dibenzothiophene4-yl)-1,1′-biphenyl-3-yl}-4,6-diphenyl-1,3,5-triazine; 5-(4′-phenyl-1,1′-biphenyl-3-yl)-12-(triphenylene-2-yl)-5H, 12H-indolo[3,2-a]carbazole

### (Group 19B)

11-(4-(1,1′-biphenyl-4-yl)-6-phenyl-1,3,5-triazine-2-yl)-12-phenyl-11H, 12H-indolo[2,3-a]carbazole; 9-(2-naphthyl)-9′-phenyl-3,3′-bis[9H-carbazole]; 11-(4,6,-bis(1,1′-biphenyl-4-yl)-1,3,5-triazine-2-yl)-12-phenyl-11H, 12H-indolo[2,3-a]carbazole; 9-(9-phenanthrenyl)-9′-phenyl-3,3′-bis[9H-carbazole]

### (Group 20B)

2-{5-(dibenzothiophene4-yl)-4′-phenyl-1,1′-biphenyl-3-yl}-4,6-diphenyl-1,3,5-triazine; 2-{5-(6-phenyldibenzothiophene4-yl)-1,1′-biphenyl-3-yl}-4,6-diphenyl-1,3,5-triazine; 2-{5-(6-(1,1′-biphenyl-4-yl)dibenzothiophene4-yl)-1,1′-biphenyl-3-yl}-4,6-diphenyl-1,3,5-triazine; 2-{5-(6-(1,1′-biphenyl-3-yl)dibenzothiophene4-yl)-1,1′-biphenyl-3-yl}-4,6-diphenyl-1,3,5-triazine; 2-{3-(6-(1,1′-biphenyl-4-yl)dibenzothiophene4-yl)phenyl}-4,6-diphenyl-1,3,5-triazine; 2-{4-(6-(1,1′-biphenyl-4-yl)dibenzothiophene4-yl)phenyl}-4,6-diphenyl-1,3,5-triazine; 2-{3-(6-(1,1′-biphenyl-4-yl)dibenzothiophene4-yl)phenyl}-4,6-bis(phenyl-d₅)-1,3,5-triazine; 2-{3-(6-(9,9-dimethylfluorene-2-yl)dibenzothiophene4-yl)phenyl}-4,6-diphenyl-1,3,5-triazine; 2-{5-(6-(9,9-dimethylfluorene-2-yl)dibenzothiophene4-yl)-1,1′-biphenyl-3-yl}-4,6-diphenyl-1,3,5-triazine; 6,6′,9,9′-tetraphenyl-3,3′-bis[9H-carbazole]; 9-(9,9-dimethylfluorene-2-yl)-9′-phenyl-3,3′-bis[9H-carbazole]; 9-(1,1′-biphenyl-4-yl)-9′-phenyl-3,3′-bis[9H-carbazole]; 9-phenyl-9′-(triphenylene-2-yl)-3,3′-bis[9H-carbazole]; 9-(1,1′-biphenyl-4-yl)-9′-(dibenzothiophene2-yl)-3,3′-bis[9H-carbazole]; 9-(dibenzothiophene2-yl)-9′-(9,9-dimethylfluorene-2-yl)-3,3′-bis[9H-carbazole]; 9,9′-bis(1,1′-biphenyl-4-yl)-3,3′-bis[9H-carbazole]; 9-(dibenzothiophene2-yl)-9′-phenyl-3,3′-bis[9H-carbazole]; 9-(dibenzothiophene4-yl)-9′-phenyl-3,3′-bis[9H-carbazole]; 9-(dibenzothiophene4-yl)-9′-phenyl-3,4′-bis[9H-carbazole]

### (Group 21B)

4-{3-(1,1′-biphenyl-4-yl)phenyl}-6-phenyl-dibenzothiophene; 2-{3-(6-(1,1′-biphenyl-4-yl)dibenzothiophene4-yl)phenyl}-4,6-diphenyl-1,3,5-triazine; 4-(triphenylene-2-yl)-6-(1,1′-biphenyl-4-yl)-dibenzothiophene; 4-{3-(1,1′-biphenyl-4-yl)phenyl}-dibenzothiophene; 1,6-bis(9H-carbazole-9-yl)-[1]benzothieno[2,3-c]pyridine; iridium(III)bis(2-phenylpyridyl-N,C²′)(4-ethyl-2,5-diphenylpyridyl-N,C₂′)

### (Group 22B)

2-{5-(dibenzothiophene4-yl)-1,1′-biphenyl-3-yl}-4,6-diphenyl-1,3,5-triazine; 2-{5-(6-phenyldibenzothiophene4-yl)-1,1′-biphenyl-3-yl}-4,6-diphenyl-1,3,5-triazine; 2-{5-(6-(1,1′-biphenyl-4-yl)dibenzothiophene4-yl)-1,1′-biphenyl-3-yl}-4,6-diphenyl-1,3,5-triazine; 2-{5-(6-(1,1′-biphenyl-3-yl)dibenzothiophene4-yl)-1,1′-biphenyl-3-yl}-4,6-diphenyl-1,3,5-triazine; 2-{5-(dibenzothiophene3-yl)-1,1′-biphenyl-3-yl}-4,6-diphenyl-1,3,5-triazine; 2-{5-(dibenzothiophene3-yl)-4′-phenyl-1,1′-biphenyl-3-yl}-4,6-diphenyl-1,3,5-triazine; 2-{3-(6-(1,1′-biphenyl-4-yl)dibenzothiophene4-yl)phenyl}-4,6-bis(phenyl-d₅)-1,3,5-triazine; 2-{3-(6-(9,9-dimethylfluorene-2-yl)dibenzothiophene4-yl)phenyl}-4,6-diphenyl-1,3,5-triazine; 2-{5-(6-(9,9-dimethylfluorene-2-yl)dibenzothiophene4-yl)-1,1′-biphenyl-3-yl}-4,6-diphenyl-1,3,5-triazine; 2-{3′-triphenylenyl-[1,1′-biphenyl]-3-yl}-4,6-diphenyl-1,3,5-triazine; 2-(6-(4′-phenyl-1,1′-biphenyl-4-yl)dibenzothiophene4-yl)-4-(1,1′-biphenyl-3-yl)-6-phenyl-1,3,5-triazine; 5-{4-(4,6-diphenyl-1,3,5-triazine-2-yl)phenyl}-8-phenyl-5H,8H-indolo[2,3-c]carbazole; 5-(1,1′-biphenyl-4-yl)-8-(4′-phenyl-1,1′-biphenyl-3-yl)-5H,8H-indolo[2,3-c]carbazole; 5-(1,1′-biphenyl-3-yl)-8-(4′-phenyl-1,1′-biphenyl-4-yl)-5H,8H-indolo[2,3-c]carbazole; 5-(4′-phenyl-1,1′-biphenyl-4-yl)-12-(1,1′-biphenyl-4-yl)-5H, 12H-indolo[3,2-a]carbazole; 5-(dibenzothiophene4-yl)-8-(4′-phenyl-1,1′-biphenyl-3-yl)-5H,8H-indolo[2,3-c]carbazole; 5-(triphenylene-2-yl)-8-(4′-phenyl-1,1′-biphenyl-4-yl)-5H,8H-indolo[2,3-c]carbazole; 5-{3-(dibenzothiophene4-yl)phenyl}-8-(4′-phenyl-1,1′-biphenyl-4-yl)-5H,8H-indolo[2,3-c]carbazole; 5-{3-(9H-carbazole-9-yl)phenyl}-8-(4′-phenyl-1,1′-biphenyl-3-yl)-5H,8H-indolo[2,3-c]carbazole; 5-(4′-phenyl-1,1′-biphenyl-4-yl)-12-(4′-phenyl-1,1′-biphenyl-3-yl)-5H, 12H-indolo[3,2-a]carbazole; 5-(4′-(phenyl-d₅)-1,1′-biphenyl-4-yl)-8-(phenyl-d₅)-5H,8H-indolo[2,3-c]carbazole; 5-(4′-phenyl-1,1′-biphenyl-3-yl)-12-phenyl-5H, 12H-indolo[3,2-a]carbazole; 5-(3′-(phenyl-d₅)-1,1′-biphenyl-4-yl)-8-(phenyl-d₅)-5H,8H-indolo[2,3-c]carbazole; 5-(4′-phenyl-1,1′-biphenyl-4-yl)-12-phenyl-5H, 12H-indolo[3,2-a]carbazole; 5-(4′-phenyl-1,1′-biphenyl-3-yl)-12-(triphenylene-2-yl)-5H, 12H-indolo[3,2-a]carbazole; 5-{3-phenyl(1,1′-biphenyl-3-yl)}-8-(4′-phenyl-1,1′-biphenyl-3-yl)-5H,8H-indolo[2,3-c]carbazole

### (Group 23B)

2,4-bis(1,1′-biphenyl-3-yl)-6-(10H-[1]benzothieno[3,2-b]indole-10-yl)-1,3,5-triazine; 3-(10-phenyl-5,10-dimethyl-5, 10-dihydroindolo[3,2-b]indole-5-yl)phenyl-1,3,5-triazine; 2-(1,1′-biphenyl-3-yl)-4-(10H-benzofuro[3,2-b]indole-10-yl)-6-phenyl-1,3,5-triazine; 9-(2-naphthyl)-9′-phenyl-3,3′-bis[9H-carbazole]; 9-(1,1′-biphenyl-3-yl)-9′-phenyl-3,3′-bis[9H-carbazole]; 3-(dibenzofuran-2-yl)-9-(5-phenyl(1,1′-biphenyl-3-yl))-9H-carbazole; 3-(dibenzothiophene2-yl)-9-(2-naphthylbenzene-3-yl)-9H-carbazole; 11-(4,6-diphenyl-1,3,5-triazine-2-yl)-12-phenyl-11H, 12H-indolo[2,3-a]carbazole; 9,9′-diphenyl-3,3′-bis[9H-carbazole]

### (Group 24B)

11-(4-(1,1′:3′,1″-terbenzene-3-yl)-6-phenyl-1,3,5-triazine-2-yl)-12-phenyl-11H, 12H-indolo[2,3-a]carbazole; 11-(4-(1,1′-biphenyl-3-yl)-6-phenyl-1,3,5-triazine-2-yl)-12-phenyl-11H, 12H-indolo[2,3-a]carbazole; 11-(4-(1,1′-biphenyl-4-yl)-6-phenyl-1,3,5-triazine-2-yl)-12-phenyl-11H, 12H-indolo[2,3-a]carbazole; 9-(1,1′-biphenyl-2-yl)-9′-(1,1′-biphenyl-3-yl)-3,3′-bis[9H-carbazole]; 9,9′-bis(1,1′-biphenyl-2-yl)-3,3′-bis[9H-carbazole]

### (Group 25B)

2-(1,1′-biphenyl-4-yl)-4-phenyl-6-(9,9′-spirobifluorene-4-yl)-1,3,5-triazine; 9-(1,1′-biphenyl-3-yl)-9′-(1,1′-biphenyl-4-yl)-3,3′-bis[9H-carbazole]; 9-phenyl-9′-(triphenylene-2-yl)-3,3′-bis[9H-carbazole]; 2-(1,1′-biphenyl-3-yl)-4-phenyl-6-(9,9′-spirobifluorene-4-yl)-1,3,5-triazine; 2,4,6-tri(1,1′-biphenyl-3-yl)-1,3,5-triazine; N,N′-diphenyl-N, N′-bis(1-naphthyl)benzidine; 4,7-diphenyl-1,10-phenanthroline; (9,9-dimethylfluorene-3-yl)(9-phenyl-9H-carbazole-3-yl)(1,1′:3′,1″-terbenzene-5′-yl)amine

### (Group 26B)

9-(7-cyanotriphenylene-2-yl)-9′-phenyl-3,3′-bis[9H-carbazole]; 9-(4′-cyano-1,1′-biphenyl-4-yl)-9′-phenyl-3,3′-bis[9H-carbazole]; 9-(5-(4,6-diphenyl-1,3,5-triazine-2-yl)-1,1′-biphenyl-3-yl)-9′-phenyl-2,3′-bis[9H-carbazole]; 9-(3-(4-(1,1′-biphenyl-3-yl)-6-phenyl-1,3,5-triazine-2-yl)phenyl)-9′-phenyl-2,3′-bis[9H-carbazole]; 9-(3′-(4,6-diphenyl-1,3,5-triazine-2-yl)-1,1′-biphenyl-3-yl)-9′-phenyl-2,3′-bis[9H-carbazole]; 9-(3-(4,6-diphenyl-1,3,5-triazine-2-yl)phenyl)-9′-phenyl-2,3′-bis[9H-carbazole]

### (Group 28B)

tris(4-(6H-6-aza-12-oxaindeno[1,2-b]fluorene-6-yl)phenyl)amine; 5-methyl-8-((4-(5-methyl-5H,8H-indolo[2,3-c]carbazole-8-yl)-phenyl)(2-pyridyl)phosphoryl)-5H,8H-indolo[2,3-c]carbazole; tetrakis(4-(6H-6-aza-8-oxaindeno[2,3-b]fluorene-6-yl)-phenyl)silane; 2,5-bis(4-(6H-6-aza-12-oxaindeno[1,2-b]fluorene-6-yl)phenyl)furan; tetrakis(4-(6H-6-aza-8-oxaindeno[2,3-b]fluorene-6-yl)-phenyl)germane; 1,4-bis(4-(7-aza-7H-12-thiaindeno[1,2-a]fluorene-7-yl)phenyl)-5,5-dimethyl-1,3-cyclopentadiene

### (Group 29B)

9-(7-cyanotriphenylene-2-yl)-9′-phenyl-3,3′-bis[9H-carbazole]; 9-(3-(4,6-diphenyl-1,3,5-triazine-2-yl)phenyl)-9′-phenyl-2,3′-bis[9H-carbazole]; 9-(4′-cyano-1,1′-biphenyl-4-yl)-9′-phenyl-3,3′-bis[9H-carbazole]

### (Group 30B)

1,3-bis(9H-carbazole-9-yl)benzene; bis[4-[9,9-dimethylacridine-10(9H)-yl]phenyl]sulfone; 4,4′-bis(9-carbazolyl)-1,1′-biphenyl; 4,4′-bis(9-carbazolyl)-1,1′-benzophenone; bis(2-((oxo)diphenylphosphino)phenyl)ether; 4,4′-bis(9-dimethoxycarbazole)-1,1′-benzophenone

### (Group 31B)

11-(4-(1,1′-biphenyl-4-yl)-6-phenyl-1,3,5-triazine-2-yl)-12-phenyl-11H, 12H-indolo[2,3-a]carbazole; 11-(4-(3′-phenyl-1,1′-biphenyl-3-yl)-6-phenyl-1,3,5-triazine-2-yl)-12-phenyl-11H, 12H-indolo[2,3-a]carbazole; 11-(4-(3′-phenyl-1,1′-biphenyl-4-yl)-6-phenyl-1,3,5-triazine-2-yl)-12-phenyl-11H, 12H-indolo[2,3-a]carbazole; 11-(4,6-diphenyl-1,3,5-triazine-2-yl)-12-(4′-phenyl-1,1′-biphenyl-4-yl)-11H, 12H-indolo[2,3-a]carbazole; 11-(4,6-bis(1,1′-biphenyl-4-yl)-1,3,5-triazine-2-yl)-12-phenyl-11H, 12H-indolo[2,3-a]carbazole; 11-(4-(1,1′-biphenyl-4-yl)-6-phenyl-1,3,5-triazine-2-yl)-12-(1,1′-biphenyl-3-yl)-11H, 12H-indolo[2,3-a]carbazole; 9-(1,1′-biphenyl-3-yl)-9′-phenyl-3,3′-bis[9H-carbazole]; 9-(1,1′-biphenyl-4-yl)-9′-phenyl-3,3′-bis[9H-carbazole]; 9-(1,1′-biphenyl-3-yl)-9′-(1,1′-biphenyl-4-yl)-3,3′-bis[9H-carbazole]

### (Group 32B)

2-(1,1′-biphenyl-3-yl)-4-[3-(9H-carbazole-9-yl)phenyl]-6-(dibenzofuran-3-yl)-1,3,5-triazine; iridium(III)tris(5′-phenyl-2-phenylpyridyl-N, C₂′); 8-(4-(4,6-di(naphthalene-2-yl)-1,3,5-triazine-2-yl)phenyl)quinoline; Liq; 2-(1,1′-biphenyl-3-yl)-4-[3-(9H-carbazole-9-yl)phenyl]-6-(dibenzofuran-4-yl)-1,3,5-triazine; 2-[3′-(9H-carbazole-9-yl)-1,1′-biphenyl-4-yl]-4-(dibenzofuran-3-yl)-6-phenyl-1,3,5-triazine; 2-[4′-(9H-carbazole-9-yl)-1,1′-biphenyl-3-yl]-4-(dibenzofuran-3-yl)-6-phenyl-1,3,5-triazine; 2-(1,1′-biphenyl-4-yl)-4-[3-(9H-carbazole-9-yl)phenyl]-6-(dibenzofuran-3-yl)-1,3,5-triazine; 2-(1,1′-biphenyl-4-yl)-4-[4-(9H-carbazole-9-yl)phenyl]-6-(dibenzofuran-3-yl)-1,3,5-triazine; 2-[3-(9H-carbazole-9-yl)phenyl]-4-(dibenzofuran-3-yl)-6-phenyl-1,3,5-triazine; 2-[4-(9H-carbazole-9-yl)phenyl]-4-(dibenzofuran-3-yl)-6-phenyl-1,3,5-triazine; 2-[3-(3-phenyl-9H-carbazole-9-yl)phenyl]-4-(dibenzofuran-3-yl)-6-phenyl-1,3,5-triazine; 2-[4-(3-phenyl-9H-carbazole-9-yl)phenyl]-4-(dibenzofuran-3-yl)-6-phenyl-1,3,5-triazine; 9,9′-bis(1,1′-biphenyl-4-yl)-3,3′-bis[9H-carbazole]; 5,8-bis(1,1′-biphenyl-4-yl)-5H,8H-indolo[2,3-c]carbazole; 2-(3-phenyl-9H-carbazole-9-yl)-4-(dibenzofuran-3-yl)-6-phenyl-1,3,5-triazine; 2-[3-(9H-carbazole-9-yl)phenyl]-4-phenyl-6-(dibenzofuran-3-yl)-pyrimidine

### (Group 33B)

2,8-bis(trifluoromethoxy)-10,12-bis(dicyanomethylene)-10,12-dihydroindeno[2,1-b]fluorene; N-phenyl-N-(1,1′:4′1,1″-terphenyl4-yl)-N′,N′-bis(1,1′-biphenyl-4-yl)-1,1′-biphenyl-4,4′-diamine; N,N-bis(4-(dibenzofuran-4-yl)phenyl)[1,1′:4′,1″-terphenyl]-4-amine; biphenyl-2-ylbis(9,9′-spirobi[9H-fluorene]-2-yl)amine; 2,8-bis(3,5-bis(trifluoromethyl)phenyl)-10,12-bis(dicyanomethylene)-10,12-dihydroindeno[1,2-b]fluorene; N-(naphthalene-1-yl)-N-phenyl-N′,N′-bis(1,1′-biphenyl-4-yl)-1,1′-biphenyl-4,4′-diamine; N,N-bis(1,1′-biphenyl-4-yl)-4-(9-phenyl-9H-carbazole-3-yl)aniline; N, N-bis(biphenyl-4-yl)-3′-(9H-carbazole-9-yl)-1,1′-biphenyl-4-amine; N-biphenyl-2-yl-N-9,9-dimethylfluorene-2-yl-(9,9′-spirobi[9H-fluorene]-2-yl)amine; 6,12-bis(dicyanomethylene)-2,8-bis(3-trifluoromethyl-4-fluorophenyl)-5,11-diazaindeno[1,2-b]fluorene; biphenyl-2-ylbis(9,9′-diphenyl-9H-fluorene-2-yl)amine; N-(1,1′-biphenyl-4-yl)-N-(9,9-dimethylfluorene-2-yl)-4-(9-phenyl-9H-carbazole-3-yl)aniline

### (Group 34B)

9-(1-naphthyl)-10-(2-naphthyl)anthracene; 9-(benzo[b]naphtho[2,3-d]furan-7-yl)-10-phenylanthracene; 9-(benzo[b]naphtho[2,3-d]furan-7-yl)-10-phenylanthracene-d8; 9-(benzo[b]naphtho[2,3-d]furan-7-yl-d9)-10-(phenyl-d5)anthracene-d8; 9-(benzo[b]naphtho[2,1-d]furan-7-yl)-10-(phenyl-d5)anthracene; 9-(1-naphthyl)-10-(4-(2-naphthyl)phenyl)anthracene; 9-(benzo[b]naphtho[2,1-d]furan-7-yl)-10-(1,1′-biphenyl-3-yl)anthracene-d8; 9-(1-naphthyl-d7)-10-(4-(2-naphthyl)phenyl-d11)anthracene-d8; 9-(benzo[b]naphtho[2,3-d]furan-7-yl)-10-(phenyl-d5)anthracene; 9-(benzo[b]naphtho[2,1-d]furan-7-yl)-10-phenylanthracene; 9-(benzo[b]naphtho[2,1-d]furan-7-yl)-10-(1,1′-biphenyl)anthracene; 9-(benzo[b]naphtho[2,3-d]furan-7-yl)-10-(1-naphthyl)anthracene; 9-(benzo[b]naphtho[2,3-d]furan-7-yl)-10-(1,1′-biphenyl-3-yl)anthracene

### (Group 35B)

9-(1-naphthyl)-10-(4-(2-naphthyl)phenyl)anthracene; 9-(1-naphthyl)-10-(3-(2-naphthyl)phenyl)anthracene; 9-(2-naphthyl)-10-(3-(2-naphthyl)phenyl)anthracene

In one embodiment, as the first component and the second component, one or more compounds selected from compounds corresponding to the first compound and the second compound in the mixture A can be independently used. The first component and the second component in the present specification may not be independently the first compound and the second compound in the mixture A.

### <Amount Proportions of First Component and Second Component, and the Like>

The amount proportions of the first component and the second component in the mixed powder are not particularly limited, and they may be appropriately adjusted depending on the purpose.

In one embodiment, when MOL1 [mol %] is used as the molar concentration of the first component in the mixed powder and MOL2 [mol %] is used as the molar concentration of the second component in the mixed powder, MOL1 [mol %] and MOL2 [mol %] satisfy the following formula (1):

$$0 < MOL 2 / ( MOL 1 + M O L 2 ) ≦ 0.2 . \qquad ( 1 )$$

The formula (1) means a mixed material in which the second component has a certain amount less. The amount of the second component based on the total amount of the first component and the second component (MOL2/(MOL1+MOL2) may be for example, 0.15 or less, 0.10 or less, or 0.05 or less. Further, MOL2/(MOL1+MOL2) may be for example, 0.001 or more, or 0.005 or more. The range of MOL2/(MOL1+MOL2) can also be expressed as in the following formulas (1-1) to (1-3).

$$0 < MOL 2 / ( MOL 1 + M O L 2 ) ≦ 0 \text{.15} \qquad ( 1 - 1 )$$

$$0 < MOL 2 / ( MOL 1 + M O L 2 ) ≦ 0 \text{.10} \qquad ( 1 - 2 )$$

$$0 < MOL 2 / ( MOL 1 + M O L 2 ) ≦ 0.05 \qquad ( 1 - 3 )$$

The amount of the first component based on the total amount of the first component and the second component (MOL1/(MOL1+MOL2)) is 0.80 or more and less than 1.00, and it may be for example, 0.85 or more, 0.90 or more, 0.95 or more, or 0.99 or more. Further, MOL1/(MOL1+MOL2) may be for example, 0.999 or less, or 0.995 or less. The range of MOL1/(MOL1+MOL2) can also be expressed as in the following formulas (1-11) to (1-15).

$$0.80 ≦ MOL 1 / ( MOL 1 + M O L 2 ) < 1. \qquad ( 1 - 11 )$$

$$0.85 ≦ MOL 1 / ( MOL 1 + M O L 2 ) < 1 \text{.00} \qquad ( 1 - 12 )$$

$$0.9 ≦ MOL 1 / ( MOL 1 + M O L 2 ) < 1 \text{.00} \qquad ( 1 - 13 )$$

$$0.95 ≦ MOL 1 / ( MOL 1 + M O L 2 ) < 1 \text{.00} \qquad ( 1 - 14 )$$

$$0.99 ≦ MOL 1 / ( MOL 1 + M O L 2 ) < 1 \text{.00} \qquad ( 1 - 15 )$$

In one embodiment, when MOL1 and MOL2 satisfy the above formula (1), when M1 [kg/mol] is used as the molecular weight of the first component, when P1 [Pa] is used as the vapor pressure obtained by ThermoGravimetry-Differential Thermal Analysis of the first component at arbitrary temperature, when M2 [kg/mol] is used as the molecular weight of the second component, and when P2 [Pa] is used as the vapor pressure obtained by ThermoGravimetry-Differential Thermal Analysis of the second component at arbitrary temperature, the temperature T1 [° C.] of the first component in the case of P1/M1^{1/2}=0.04×{MOL1/(MOL1+MOL2)} and the temperature T2 [° C.] of the second component in the case of P2/M2^{1/2}=0.04×{MOL2/(MOL1+MOL2)} satisfy the following formula (2):

$$- 2 {0}^{°} C . ≦ T 1 - T 2 ≦ 4 {0}^{°} C . \qquad ( 2 )$$

As described above, in conventional premix technique, there is a problem that when a plurality of substrates is continuously subjected to deposition, the mixing proportion thereof is variable depending on the substrate. However, according to the inventors' study, these problems were found to be more pronounced in, for example, as premix materials of the host compound and the dopant compound, a system that there are differences in the amount proportion of constituent materials, and one proportion is large and the other proportion is small.

When the mixed powder according to an aspect of the present invention described above is used, the variability of the component proportion can be suppressed in the mixed film during the film formation process in the deposition process. Although this effect is fully exhibited even in the system, like above, that, regardless of the amount proportion of constituent materials, one proportion is large and the other proportion is small, the mixed powder satisfies the above formula (2) to obtain a greater effect of suppressing the variability of the component proportion even in this system.

The formula (2) expresses that in the system where the mixing proportion of one compound is smaller than a certain level, when a compound with different deposition characteristics to a certain extent is daringly used in combination thereto, the variability of the component proportion can be more reliably suppressed during the deposition process.

Hereinafter, the formula (2) will be described in detail.

When thermogravimetric analysis is conducted on one organic compound, the mass change (loss) amount [kg] is shown against temperature change, and then the factor of the time [min] is added thereto to be able to obtain the mass loss rate (rate) [kg/min]. Since the mass loss rate represents the mass vaporized (deposited) per unit time, it can be an indicator of the deposition characteristics of the organic compound, but since it is used in the present invention as the indicator which is further difficult to depend on the molecular weight of the compound, the molar deposition rate [mol/min] is used as an index of deposition characteristics.

Here, it is known that the relationship represented by the following formula (A1) is provided between mass loss rate m, vapor pressure (saturated vapor pressure) P, and molecular weight M of the organic compound, and since the mass loss rate m is proportional to P×M^{1/2}, the molar deposition rate m/M is proportional to P×M^{1/2}/M, that is, P/M^{1/2}. That is, it can be seen that the molar deposition rate can be evaluated by P/M^{1/2}. The unit of P/M^{1/2} is [Pa/(kg/mol)^{1/2}].

$$m = P \sqrt{\frac{M}{2 π R T}} \qquad ( A1 )$$

In the formula (A1), each symbol indicates the following matter.

m: mass loss rate [kg/(s·m²)]

(The unit (s·m²) means time (s)× deposition area (m²) on substrate surface.)
P: saturated vapor pressure [Pa]
M: molecular weight of the organic compound [kg/mol]
R: gas constant [J/(mol·K)]
T: temperature [K]

P/M^{1/2} obtained based on thermogravimetric analysis and being proportional to molar deposition rate is used as an indicator of deposition characteristics, and in addition thereto, one of the features of the present invention is that the present inventors focus on the relationship between the blending ratio (molar ratio) of two components used in the mixed powder and the ratio of P/M^{1/2}. Specifically, the present invention is based on the technical idea that in the system where the mixing ratio of one compound is smaller than a certain level, when the blending ratio (molar ratio) of the two components in the mixed powder and the ratio of P/M^{1/2} of the two components are approximated, it is possible to maintain the balance of vaporized amounts of two components for a long time in the deposition processes (especially continuous deposition processes). The formula (2) expresses those described above.

The relationship between the above technical idea and the formula (2) will be described below using the specific examples.

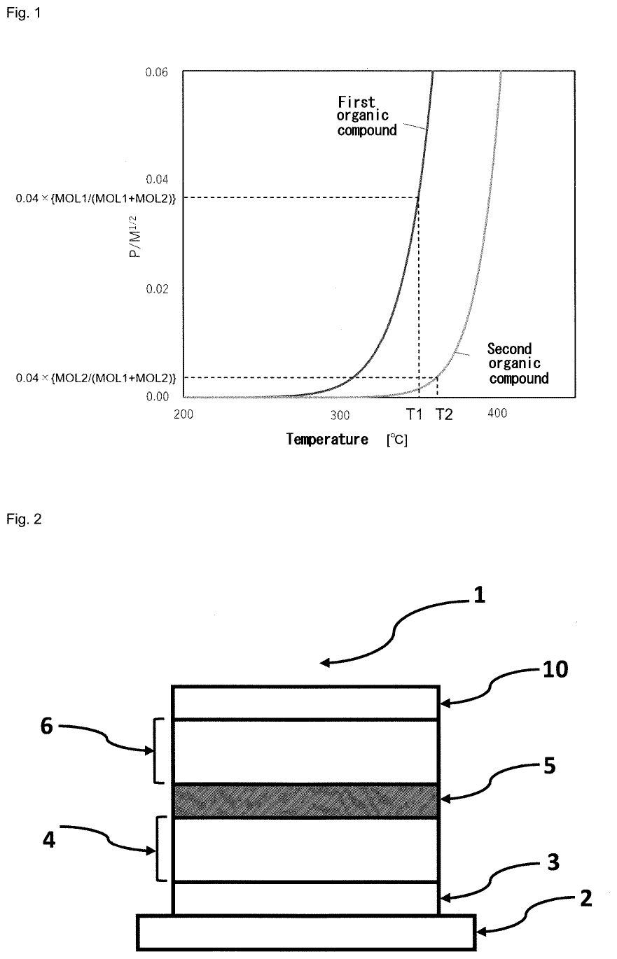

As an example of the mixed powder according to one aspect of the present invention, The mixed powder including the first component (major component) and the second component (minor component) is assumed, and the deposition characteristic curve of temperature and P/M^{1/2} shown in FIG. 1 is assumed as each deposition characteristic. The molar ratio of the two components in this example is 9:1, that is, MOL2/(MOL1+MOL2) is 0.1. Correspondingly, when the ratio of P/M^{1/2} of the two components is close to 9:1, the balance of vaporization amount (deposition amount) is maintained for a long time, and therefore, the formula (2) expresses this condition in terms of a temperature perspective.

That is, the formula (2) indicates that the present inventors focus on the difference of the temperature T1 in the case that P/M^{1/2} of the first component reaches the specific value (0.04×{MOL1/(MOL1+MOL2)}) corresponding to the molar ratio thereof (MOL1/(MOL1+MOL2)) and the temperature T2 in the case that P/M^{1/2} of the second component reaches the specific value (0.04×{MOL2/(MOL1+MOL2)}) corresponding to the molar ratio thereof (MOL2/(MOL1+MOL2)), and that when the temperature difference (T1−T2) is within a predetermined range, the above-mentioned effects can be fully exhibited (here, when the temperature difference (T1−T2) is 0° C., the present inventors focus on a point that the ratio of the molar deposition rate of the first component at temperature T1=T2 and the molar deposition rate of second component perfectly matches 9:1 which is the molar ratio of the above two components).

In this example, as shown in FIG. 1, since the difference between T1 and T2 (T1−T2) is −10° C. which is within the specified range, the mixed powder is put into the deposition source and then the deposition is conducted to be able to maintain the balance of the vaporized amounts of these two components for a long time.

The value “0.04” is a constant set in consideration of conditions such as the molecular weight and vapor pressure of the compound generally employed in the deposition process of the organic compound.

Here, the reason for using the difference between T1 and T2 (T1−T2) as “−20° C.≤T1−T2≤ 40° C.” will be described below.

As described above, when T1 and T2 are almost the same or within a certain approximate range, it is possible to maintain the balance of the vaporization amount of the two components for a long time. However, as a result of further studies by the inventors, in particular, when considering reducing the difference in the molar ratio of the two components between the mixed powder and the deposited film, that is, improving the reproducibility of the component ratio, it has been found that better results can be obtained when T1 is higher than T2 by a certain degree than when T1 and T2 are the same. The reason for this is not necessarily certain, but it is thought to be related to the fact that the second component (the small amount of component) is exposed to the outside in a small amount and is less likely to vaporize than the first component. This is considered, and then one in which conditions are selected so that the first component side is also slightly difficult to be vaporized and they are modified is the above numerical range.

A different idea from the present invention may be that it is preferable to match the molar deposition rate at arbitrary temperature, regardless of the mixing ratio of the mixed powder. That is, it is the idea that a combination of the compounds in which the deposition characteristic curves overlap or are very close to each other is preferable. In this idea, a combination in which the deposition characteristic curves are deviated as shown in those of the first component and the second component of FIG. 1 is not preferable.

The difference between T1 and T2 (T1−T2) is, for example, −15° C. or more, −10° C. or more, −5° C. or more, 0° C. or more, or 5° C. or more, and is, for example, 35° C. or less, 30° C. or less, 25° C. or less, 20° C. or less, or 15° C. or less.

In one embodiment, the difference between T1 and T2 (T1−T2) is −10° C. or more and 30° C. or less, −5° C. or more and 25° C. or less, or 0° C. or more and 20° C. or less.

In one embodiment, the difference between T1 and T2 (T1−T2) can also be expressed as in the following formulas (2-1) to (2-2).

$${0}^{∘} C . ≦ T 1 - T 2 ≦ 4 {0}^{∘} C . \qquad ( 2 - 1 )$$

$${5}^{∘} C . ≦ T 1 - T 2 ≦ 4 {0}^{∘} C . \qquad ( 2 - 2 )$$

When they are within the above-mentioned range, the difference in the molar ratio of the two components in the mixed powder and the deposited film can be reduced, and the reproducibility of the component ratio can be improved.

### (Method for Measuring and Calculating T1 and T2)

The temperature T1 in the case where P/M^{1/2} of the first component is 0.04×{MOL1/(MOL1+MOL2)} and the temperature T2 in the case where P/M^{1/2} of the second component is 0.04×{MOL2/(MOL1+MOL2)} are determined by reading from the deposition characteristic curve of temperature and P/M^{1/2} of each compound. The specific method is shown below.

The deposition characteristic curve of temperature and P/M^{1/2} of each compound of the first component and the second component is obtained by the following method.

### (ThermoGravimetry-Differential Thermal Analysis)

The amount of weight loss is measured using an apparatus for ThermoGravimetry-Differential Thermal Analysis in the case where the temperature is changed under vacuum. The ThermoGravimetry-Differential Thermal Analysis (TG-DTA) is a method in which the change in mass of a sample is continuously measured when the sample is heated, and is used to detect physical changes which involve mass changes such as sublimation and evaporation.

The measurement is conducted under the following measurement conditions.

### (Measurement Condition)

Apparatus: apparatus for ThermoGravimetry-Differential Thermal Analysis
Sample container: aluminum nitride pan
Mass of sample: 3 to 10 mg
Heating rate: 3° C./min
Measurement range:35° C. or more, 500° C. or less
Degree of vacuum: 1×10⁻³ Pa or less

### (Conversion to Deposition Characteristic Curve of Temperature and P/M^{1/2}))

P/M^{1/2} at each temperature is calculated from the mass loss rate (kg/s) at each temperature obtained by the ThermoGravimetry-Differential Thermal Analysis using the following conversion formula (A11).

$$\frac{P}{\sqrt{M}} = \frac{\sqrt{2 π RT}}{MA} \cdot \frac{d Δ W}{dT} \cdot \frac{dT}{dt} \qquad ( A11 )$$

In the formula (A11), each symbol indicates the following matter.
P: saturated vapor pressure [Pa]
A: area of vaporization surface in sample container [m²]
ΔW: mass loss amount of the organic compound in the sample container [kg]
T: temperature of the vaporization surface in the sample container [K]
t: time [s]
M: molecular weight of the organic compound [kg/mol]
R: gas constant [J/(mol·K)]

P/M^{1/2} calculated is used as the vertical axis, the temperature is used as the horizontal axis, and then the deposition characteristic curve of temperature and P/M^{1/2} of the compound being a measurement target is obtained.

Strictly theoretically, the condensation coefficient “a” can affect the saturated vapor pressure P, but since in this measurement, it is thought that diffusion is sufficiently promoted in the measurement temperature range of the material, the condensation coefficient “a” is assumed to be 1. As a result, the condensation coefficient “a” does not described in the formula (A1).

### (Extrapolation of Deposition Characteristic Curve of Temperature and P/M^{1/2})

The deposition characteristic curve of temperature and P/M^{1/2} obtained above is extrapolated in calculating T1 and T2 as necessary using the following method.

The obtained deposition characteristic curve is fitted by using an exponential function of the following formula (A12).

$$\frac{P}{\sqrt{M}} = \frac{1}{\sqrt{M}} \cdot Exp ( {C}_{1} ) \cdot Exp ( - \frac{{C}_{2}}{T} ) \qquad ( A12 )$$

In the formula (A12), P, M and T is the same as described in those of the formula (A11).

The temperature is used as the horizontal axis, the natural logarithm of the saturated vapor pressure is used as the vertical axis, and then each of the intercept C₁ and the slope C₂ in the formula (2) is calculated by the least squares method for an arbitrary range where the linearity is highest. The obtained C₁ and C₂, and formula (A12) are used to be able to obtain P/M^{1/2} at arbitrary temperature.

In one embodiment, the amount of the second component is 20% by mass or less, 10% by mass or less, or 5% by mass or less based on the total amount of the first component and the second component.

In one embodiment, the amount of the second component is 0.1% by mass or more, 0.5% by mass or more, or 1% by mass or more based on the total amount of the first component and the second component.

### <5% Weight Loss Temperature>

In one embodiment, in the case where the amount of the second component is low to a certain extent, that is, in the case where the mixed powder satisfies the above (1) or the amount of the second component is 20% by mass or less based on the total of the first component and the second component, when the combination of the compounds is selected among the combinations of the first component and the second component such that the 5% weight loss temperature T_{d5}1 of the first component is lower than the 5% weight loss temperature T_{d5}2 of the second component, it is easier to suppress the variability of the balance of vaporized amounts of both components during the deposition process.

In a system where the amount of the second component is low to a certain extent, when T_{d5}1 is lower than T_{d5}2, the second component should be hardly vaporized at the temperature at which the first component begins to be vaporized, but in the case of the mixed powder according to an aspect of the present invention, the first component and the second component are in a state where they are intertwined with each other at the molecular level, and therefore, it is thought that the second component is pulled by the first component, which is present in large quantities around it, and is vaporized with less thermal energy. As a result, even when T_{d5}1 is lower than T_{d5}2, the balance of the vaporization amounts of both components can be maintained for a long time in the deposition process.

In one embodiment, T_{d5}1 is lower than T_{d5}2 by 10° C. or more, 20° C. or more, or 30 or more.

The 5% weight loss temperature (measured value under normal pressure) is measured by using the ThermoGravimetry-Differential Thermal Analysis. Specifically, it is measured by the method described in the Examples.

### <Other Component, and the Like>

The mixed powder according to an aspect of the present invention may or may not include a component other than the first component and the second component.

In one embodiment, the mixed powder consists of the first component and the second component, or substantially consists of the first component and the second component. In the latter case, unavoidable impurities may be included therein.

In one embodiment, 80% by mass or more, 85% by mass or more, 90% by mass or more, 95% by mass or more, 99% by mass or more, 99.5% by mass or more, 99.9% by mass or more, 99.99% by mass or more, or 100% by mass of the mixed powder is the first component and the second component.

In one embodiment, 80 mol % or more, 90 mol % or more, 95 mol % or more, 99 mol % or more, 99.5 mol % or more, 99.9 mol % or more, 99.99 mol % or more, or 100 mol % of the mixed powder is the first component and the second component.

In one embodiment, the mixed powder consists of the first component, the second component and a third component, or substantially consists of the first component, the second component and a third component. In the latter case, unavoidable impurities may be included therein. Here, the third component is an arbitrary organic compound (third organic compound).

In one embodiment, 80% by mass or more, 85% by mass or more, 90% by mass or more, 95% by mass or more, 99% by mass or more, 99.5% by mass or more, 99.9% by mass or more, or 99.99% by mass or more of the mixed powder is the first component and the second component, and the remaining thereof is the third component. Here, the third component is an arbitrary organic compound (third organic compound).

In one embodiment, 80 mol % or more, 90 mol % or more, 95 mol % or more, 99 mol % or more, 99.5 mol % or more, 99.9 mol % or more, or 99.99 mol % or more of the mixed powder is the first component and the second component, and the remaining thereof is the third component. Here, the third component is an arbitrary organic compound (third organic compound).

The third component is not particularly limited.

The third component has, for example, the fused ring structure having the most rings and having three or more rings, in fused ring structures included therein, and a compound different from the first component and the second component can be used therefor.

In one embodiment, when the amount of the third component is 30% by mass or more in the mixed powder, a compound having the fused ring structure having the most rings and having three or more rings, in fused ring structures included therein and being different from the first component and the second component is used as the third component.

The same compounds as those for the first component can be used as the compound having 3 or more of maximum number of rings.

### <Mixed Powder and Method for Producing the Same>

A mixed powder according to an aspect of the present invention may contain the first component and the second component in one particle, or may be a mixture of a particle composed of the first component and a particle composed of the second component.

A method for producing mixed powder according to an aspect of the present invention, for example, may include pulverizing and mixing the first component and the second component by using a mortar and the like. In the case of pulverization and mixing, when the amount of material used is large, such as when used in a large-scale deposition machine, pulverization and mixing using a pulverizer may be used.

Further, it may include putting the first component and the second component in a container and the like, heating and melting them under a chemically inert environment, subsequently, cooling them to the ambient temperature, and pulverizing the obtained mixture by using a mixer and the like to obtain powder. The latter method can lead mixing the first component and the second component at the molecular level. As a result, it can prevent problems such as mix unevenness which can be caused during transport of the mixed powder.

The mixed powder may be compressed and formed to be pellet-like.

### [Method for Deposition]

A method for deposition according to an aspect of the present invention includes: heating and vaporizing the mixed powder according to an aspect of the present invention described above in the same deposition source to form a film on the deposition surface of the opposing substrate arranged thereabove. In the method for deposition, other conditions are not particularly limited, and general apparatus and conditions can be used as a deposition apparatus and a deposition condition.

The deposition is normally conducted under vacuum (at a pressure lower than pressure of the atmosphere). The pressure inside the apparatus during deposition is preferably 5.0 Pa or less, and more preferably 1.0 Pa or less. The heating temperature during deposition is generally 150° C. to 400° C., and is preferably 200 to 350° C.

The method for deposition according to an aspect of the present invention may include: melting and mixing the mixed powder which is the mixture of a particle composed of the first component and a particle composed of the second component to use the mixed powder obtained by crushing the solidified material for the deposition.

Further, the mixed powder may be compressed in advance to form pellets as described above, and the pellets may be introduced into the deposition source to conduct the deposition.

### [Applicable Technical Field]

The mixed powder and method for deposition according to an aspect of the present invention can be used for any technical field including depositing an organic compound to form a film, and for example, the fabrication of an organic semiconductor device such as an organic EL device, an organic transistor and an organic solar cell can be applied. As an example, a method for fabricating an organic EL element using the mixed powder according to an aspect of the present invention will be described below.

### [Method for Fabricating Organic EL Device]

A method for fabricating an organic EL element in an aspect of the present invention can also be expressed as follows:

a method for fabricating an organic electroluminescence device, wherein the organic electroluminescence device includes a cathode, an anode, and one or two or more organic layers arranged between the cathode and the anode, and including an emitting layer, and the method includes: depositing a mixed powder by heating and vaporizing the mixed powder which includes a first compound and a second compound, which is solid at ordinary temperatures and pressures, and in which the first compound has the fused ring structure having the most rings and having three or more rings, in fused ring structures included therein, and the second compound has the fused ring structure having the most rings and having 5 or more rings, in fused ring structures included therein, and the fused ring structure having the most rings thereof has more rings than that of the first compound in a deposition source to form at least one layer of the one or two or more organic layers.

A mixed powder and a method for deposition in the method for the fabrication are the same as described in those of the mixed powder and the method for deposition in an aspect of the present invention described above.

As the device configuration of the organic EL device, structures in which structures of the following (1) to (4) and the like are stacked on a substrate can be given:
(1) an anode/an emitting layer/a cathode,
(2) an anode/a hole-transporting region/an emitting layer/a cathode,
(3) an anode/an emitting layer/an electron-transporting region/a cathode,
(4) an anode/a hole-transporting region/an emitting layer/an electron-transporting region/a cathode,
wherein “/” indicates that the layers are stacked adjacent to each other.

An electron-transporting region is a region composed of one or two or more organic layers (also referred to as “electron-transporting layer” and/or “electron-injecting layer”) including the electron-transporting compound, and a hole-transporting region is a region composed of one or two or more organic layers (also referred to as “hole-transporting layer” and/or “hole-injecting layer”) including the hole-transporting compound.

A schematic configuration of the organic EL device will be described with reference to FIG. 2. The organic EL device 1 includes a substrate 2, an anode 3, an emitting layer 5, a cathode 10, a hole-transporting region 4 between the anode 3 and the emitting layer 5, and an electron-transporting region 6 between the emitting layer 5 and the cathode 10.

The method for fabricating an organic EL element in an aspect of the present invention include: forming at least one layer of the organic layers in the organic EL element by the deposition method using the mixed powder. The layer formed using the mixed powder is not particularly limited, and it may be any of the organic layers. Further, two or more of the organic layers may be formed by the deposition using the mixed powder.

Examples of the combination of the first component and the second component [first component, second component] in the mixed powder used for forming the organic layer include [the host material for an emitting layer, the dopant material for an emitting layer], [the host material for a fluorescent emitting layer, the dopant material for a fluorescent emitting layer], [the hole-injecting compound, the hole-transporting compound], [the host material for a fluorescent emitting layer, the host material for a fluorescent emitting layer], and the like, but it is not limited to these.

The method for forming each layer other than the layer formed using the deposition of the mixed powder is not particularly limited, and a method for forming each layer such as a vacuum deposition process and a spin coating process using a single material can be used.

Each layer of the organic EL device will be described below.

### (Substrate)

The substrate is used as a support of an emitting device. As the substrate, glass, quartz, plastic or the like can be used, for example. Further, a flexible substrate may be used. The term “flexible substrate” means a bendable (flexible) substrate, and specific examples thereof include a plastic substrate formed of polycarbonate, polyvinyl chloride or the like.

### (Anode)

For the anode formed on the substrate, metals, alloys, electrically conductive compounds, mixtures thereof, and the like, which have large work function (specifically 4.0 eV or more) are preferably used. Specific examples thereof include indium oxide-tin oxide (ITO: Indium Tin Oxide), indium oxide-tin oxide containing silicon or silicon oxide, indium oxide-zinc oxide, tungsten oxide, indium oxide containing zinc oxide, graphene, and the like. In addition thereto, specific examples thereof include gold (Au), platinum (Pt), a nitride of a metallic material (for example, titanium nitride), or the like.

### (Hole-Injecting Layer)

The hole-injecting layer is a layer containing a substance having high hole-injecting property. As the substance having high hole-injecting property, molybdenum oxide, titanium oxide, vanadium oxide, rhenium oxide, ruthenium oxide, chromium oxide, zirconium oxide, hafnium oxide, tantalum oxide, silver oxide, tungsten oxide, manganese oxide, an aromatic amine compound, a polymer compound (oligomers, dendrimers, polymers, and the like), or the like can be given.

### (Hole-Transporting Layer)

The hole-transporting layer is a layer containing a substance having high hole-transporting property. For the hole-transporting layer, an aromatic amine compound, a carbazole derivative, an anthracene derivative, or the like can be used. A polymer compound such as poly(N-vinylcarbazole) (abbreviation: PVK) and poly(4-vinyltriphenylamine) (abbreviation: PVTPA) can also be used. Provided that a substance other than the above-described substances may be used as long as the substance has higher hole-transporting property than electron-transporting property. The layer containing the substance having high hole-transporting property may be not only a single layer, but also layers in which two or more layers formed of the above-described substances are stacked.

### (Guest (Dopant) Material of Emitting Layer)

The emitting layer is a layer containing a substance having high emitting property, and various materials can be used. For example, as the substance having high emitting property, a fluorescent compound which emits fluorescence or a phosphorescent compound which emits phosphorescence can be used. The fluorescent compound is a compound which can emit from a singlet excited state, and the phosphorescent compound is a compound which can emit from a triplet excited state.

As a blue fluorescent emitting material which can be used for the emitting layer, pyrene derivatives, styrylamine derivatives, chrysene derivatives, fluoranthene derivatives, fluorene derivatives, diamine derivatives, triarylamine derivatives, and the like can be used. As a green fluorescent emitting material which can be used for the emitting layer, aromatic amine derivatives and the like can be used. As a red fluorescent emitting material which can be used for the emitting layer, tetracene derivatives, diamine derivatives and the like can be used.

As a blue phosphorescent emitting material which can be used for the emitting layer, metal complexes such as iridium complexes, osmium complexes and platinum complexes are used. As a green phosphorescent emitting material which can be used for the emitting layer, iridium complexes and the like are used. As a red phosphorescent emitting material which can be used for the emitting layer, metal complexes such as iridium complexes, platinum complexes, terbium complexes and europium complexes are used.

### (Host Material for Emitting Layer)

The emitting layer may have a constitution in which the substance having high emitting property (guest material) is dispersed in another substance (host material). As a substance for dispersing the substance having high emitting property, a variety of substances can be used, and it is preferable to use a substance having a higher lowest unoccupied molecular orbital level (LUMO level) and a lower highest occupied molecular orbital level (HOMO level) than a substance having high emitting property.

As a substance (host material) for dispersing the substance having high emitting property, 1) a metal complex such as an aluminum complex, a beryllium complex, and a zinc complex, 2) a heterocyclic compound such as an oxadiazole derivative, a benzimidazole derivative, and a phenanthroline derivative, 3) a fused aromatic compound such as a carbazole derivative, an anthracene derivative, a phenanthrene derivative, a pyrene derivative, and a chrysene derivative, and 4) an aromatic amine compound such as a triarylamine derivative and a fused polycyclic aromatic amine derivative are used.

### (Electron-Transporting Layer)

The electron-transporting layer is a layer containing a substance having high electron-transporting property. For the electron-transporting layer, 1) a metal complex such as an aluminum complex, a beryllium complex, and a zinc complex; 2) a heteroaromatic complex such as an imidazole derivative, a benzimidazole derivative, an azine derivative, carbazole derivative, and a phenanthroline derivative; and 3) a polymer compound can be used.

### (Electron-Injecting Layer)

The electron-injecting layer is a layer containing a substance having high electron-injecting property. For the electron-injecting layer, lithium (Li), ytterbium (Yb), lithium fluoride (LiF), cesium fluoride (CsF), calcium fluoride (CaF₂), a metal complex compound such as 8-hydroxyquinolinolato-lithium (Liq), an alkali metal such as lithium oxide (LiOₓ), an alkaline earth metal, or a compound thereof can be used.

### (Cathode)

For the cathode, metals, alloys, electrically conductive compounds, mixtures thereof, and the like, which have small work function (specifically 3.8 eV or less) are preferably used. Specific examples of such a cathode material include an element belonging to Group 1 or Group 2 of the Periodic Table of the Elements, i.e., an alkali metal such as lithium (Li) and cesium (Cs), an alkaline earth metal such as magnesium (Mg), calcium (Ca) and strontium (Sr), and an alloy containing these (e.g., MgAg and AILi); a rare earth metal such as europium (Eu) and ytterbium (Yb), and an alloy containing these.

The cathode is usually formed by a vacuum deposition method or a sputtering method. Further, when silver paste or the like is used, it is possible to use the coating method, the inkjet method or the like.

When the electron-injecting layer is provided, the cathode can be formed using various conductive materials such as aluminum, silver, ITO, graphene, indium oxide-tin oxide containing silicon or silicon oxide, regardless of the work function value.

The thickness of each layer described above is not particularly limited, but is normally preferable several nm to 1 μm generally in order to suppress defects such as pinholes, to suppress applied voltages to be low, and to improve luminous efficiency.

### EXAMPLES

Hereinafter, Examples according to the present invention will be described. The present invention is not limited to these Examples. All of BH-1 to BH-3 used in Examples and Comparative Example are host materials (fluorescence) for an emitting layer in an organic EL device, all of BD-1 to BD-3 are dopant materials (fluorescence) for the emitting layer in the organic EL device, HI-1 is a hole-injecting material used in the organic EL device, and HT-1 is a hole-transporting material used in the organic EL device.

### <Compound>

Compounds used in Examples and Comparative Example are shown below.

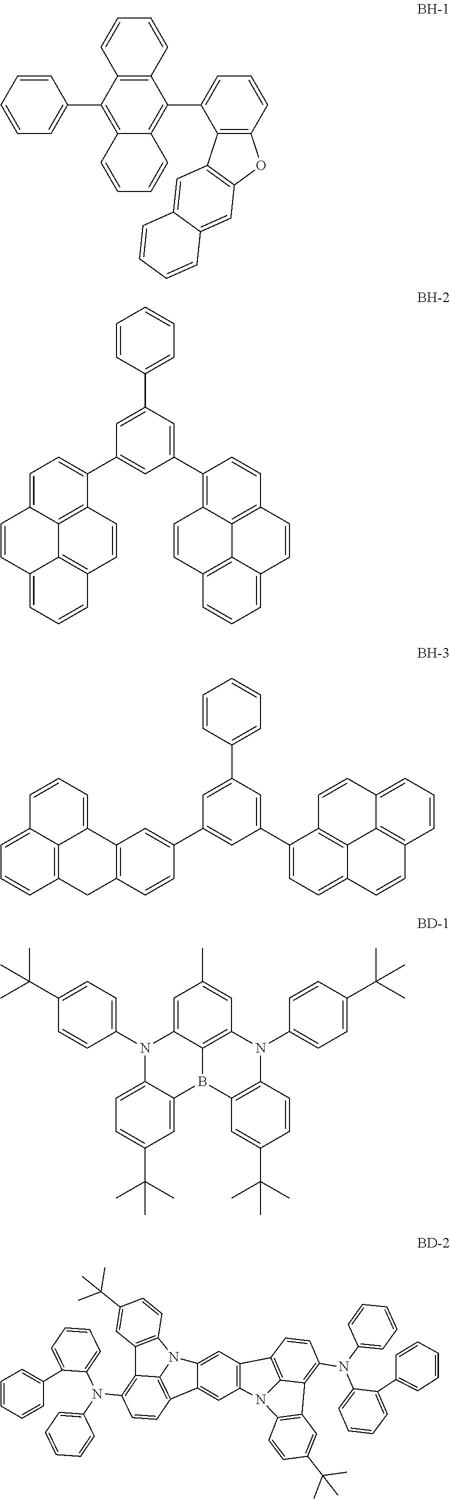

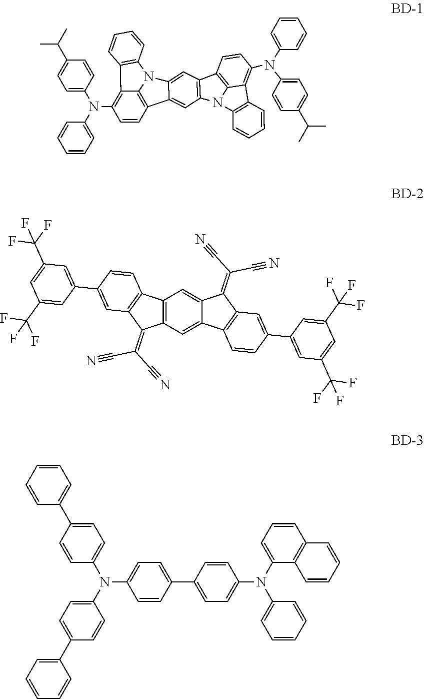 [SMILES: CC(C)c1ccc(N(c2ccccc2)c2ccc3c4cc5c(cc4n4c6ccccc6c2c34)c2ccc(N(c3ccccc3)c3ccc(C(C)C)cc3)c3c4ccccc4n5c23)cc1 ; N#CC(C#N)=C1c2cc(-c3cc(C(F)(F)F)cc(C(F)(F)F)c3)ccc2-c2cc3c(cc21)-c1ccc(-c2cc(C(F)(F)F)cc(C(F)(F)F)c2)cc1C3=C(C#N)C#N ; c1ccc(-c2ccc(N(c3ccc(-c4ccccc4)cc3)c3ccc(-c4ccc(N(c5ccccc5)c5cccc6ccccc56)cc4)cc3)cc2)cc1]

### <5% Weight Loss Temperature>

The 5% weight loss temperature of the compounds was measured under normal pressure using an apparatus for ThermoGravimetry-Differential Thermal Analysis (TG/DTA)(“STA7000RV” manufactured by Hitachi High-Tech Science Corporation). The results are shown below.
BH-1: 335° C.
BH-2: 440° C.
BH-3: 447° C.
BD-1: 375° C.
BD-2: 494° C.
BD-3: 486° C.

### <Method for Producing Mixed Powder>

The mixed powders in Examples and Comparative Example were produced by weighing each compound of the first component and the second component so that the total amount of them was 2 g in a solid state with satisfying the desired molar ratio, and by mixing them while pulverizing in a mortar.

### Example 1-1

<Production of mixed powder and measurements of T1 and T2>

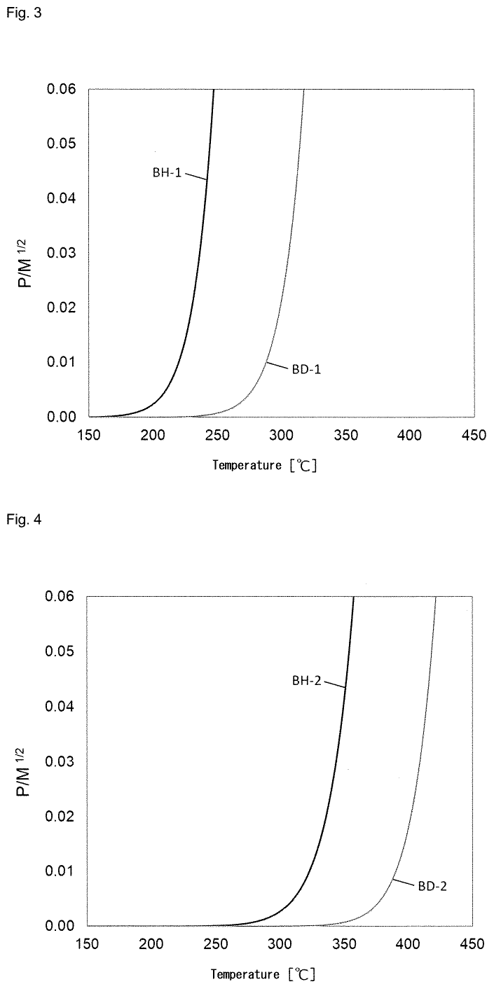

The compounds shown in Table 1 were mixed in the amounts shown in Table 1 to obtain mixed powders. The deposition characteristic curve of the temperature −P1/M1^{1/2} of BH-1 and the deposition characteristic curve of the temperature −P2/M2^{1/2} of BD-1, obtained by the method described above, were used to obtain each of the temperature T1 in the case where P1/M1^{1/2} was 0.04×{MOL1/(MOL1+MOL2)} and the temperature T2 in the case where P2/M2^{1/2} was 0.04×{MOL2/(MOL1+MOL2)}. T1, T2, and the value “T1−T2” are shown in Table 1. Further, the deposition characteristic curves of temperature and P/M^{1/2} of BH-1 and BD-1 are shown in FIG. 3. In the above case, P1 is the saturated vapor pressure of BH-1 at arbitrary temperature, P1 is the saturated vapor pressure of BD-1 at arbitrary temperature, M1 is the molecular weight of BH-1, M2 is the molecular weight of BD-1, MOL1 is the molar concentration [mol %] of BH-1 in the mixed powder, and MOL2 is the molar concentration [mol %] of BD-1 in the mixed powder.

An apparatus for ThermoGravimetry-Differential Thermal Analysis (model number “STA2500/HV”) manufactured by NETZSCH Japan K.K. was used as a measuring apparatus of ThermoGravimetry-Differential Thermal Analysis.

TABLE 1

| First component | BH-1 | |
|---|---|---|
| Second component | BD-1 | |
| MOL1 | 98.51 | mol % |
| MOL2 | 1.49 | mol % |
| M1 | 470.57 | |
| M2 | 658.78 | |
| T1 | 241° | C. |
| T2 | 247° | C. |
| T1 − T2 | −6° | C. |

### <Evaluation of Continuous Deposition>

Evaluation of continuous deposition was conducted as follows using the obtained mixed powder.

A crucible containing the mixed powder having the total amount of 0.6 g was heated in a vacuum deposition apparatus under vacuum at 1×10−4 Pa or less, the temperature thereof was adjusted so that the film formation rate was 2 Å/sec, and the film was formed by deposition on a glass substrate. The glass substrate was appropriately replaced, and then the film formation was continued. The first substrate on which a film was formed was referred to as the substrate “No. 1”, and then they after that were referred to as “No. 2”, “No. 3”, etc.

The following matters were evaluated. The results are shown in Table 2.

### · Component Ratio in Deposited Film

The molar mixing ratio of the first component and the second component was measured as follows for each deposited film formed on each substrate. The film area of the deposited film formed on the glass substrate, and the mass obtained from the film thickness in the case where the specific gravity was assumed as 1 were calculated, a solution was prepared with tetrahydrofuran (THF) solvent so that the total concentration of the mixture of the first component and the second component is 100 ppm, and the obtained solution was subjected to HPLC measurement using a high performance liquid chromatography (HPLC) apparatus (apparatus name: “LC-2040C Plus” manufactured by SHIMADZU CORPORATION) to calculate the HPLC area of each of the first component and the second component.

Separately, create a standard solution prepared with THF solvent so that the first component had a concentration of 100 ppm and the second component had a concentration of 100 ppm was obtained to calculate each peak area by HPLC measurement. The mass concentration in the solution of the first component and second component in the mixed film was calculated using the peak area value of the standard solution, and then the mass mixing ratio contained in the film was calculated using it. The mass mixing ratio was calculated using the molecular weight of each component to calculate the molar mixing ratio of each component in the mixed film.

In the column of the second component in Table 2, the numerical value shown in parentheses indicates the ratio of the molar ratio of the second component in the deposited film based on the molar ratio of the second component in the mixed powder.

### · Deposition Ratio Stability

In the case where the molar ratio of the first component in the substrate No. 1 was used as the standard value, the molar ratio of the first component in each substrate after the substrate No. 2 was calculated using the molar ratio of the first component in the substrate No. 1 as 100.0%, and it was evaluated as the deposition ratio stability. The second component was also evaluated in the same manner. That is, in the case where the molar ratio of the second component in the substrate No. 1 was used as the standard value, the molar ratio of the second component in each substrate after the substrate No. 2 was calculated using the molar ratio of the second component in the substrate No. 1 as 100.0%, and it was evaluated as the deposition ratio stability. The total film thickness deposited on the substrates No. 1 to No. 4 was 604 nm.

TABLE 2

| | Cumulative | Molar ratio | | Deposition ratio | Deposition ratio |
|---|---|---|---|---|---|
| | used amount | First | Second | stability of first | stability of second |
| Substrate No. | (% by mass) | component | component | component | component |
| Before | 0% | 98.51% | 1.49% | — | — |
| deposition | | | | | |
| (mixed powder) | | | | | |
| No. 1 | 0 to 34% | 99.40% | 0.60%(0.40) | 100.0% | 100.0% |
| No. 2 | 34 to 51% | 99.38% | 0.62%(0.41) | 100.0% | 102.4% |
| No. 3 | 51 to 67% | 99.35% | 0.65%(0.44) | 99.9% | 108.4% |
| No. 4 | 67 to 83% | 99.25% | 0.75%(0.51) | 99.8% | 125.1% |

### Example 1-2

The same compounds as those used in Example 1-1 were mixed in the amounts shown in Table 3 to obtain mixed powders. T1, T2, and the value “T1−T2” in the Example are shown in Table 3.

TABLE 3

| First component | BH-1 | |
|---|---|---|
| Second component | BD-1 | |
| MOL1 | 96.49 | mol % |
| MOL2 | 3.51 | mol % |
| M1 | 470.57 | |
| M2 | 658.78 | |
| T1 | 241° | C. |
| T2 | 259° | C. |
| T1 − T2 | −18° | C. |

The continuous deposition was conducted and evaluated using the obtained powder in the same manner as in Example 1-1. The results are shown in Table 4. The total film thickness deposited on the substrates No. 1 to No. 4 was 584 nm.

TABLE 4

| | Cumulative | Molar ratio | | Deposition ratio | Deposition ratio |
|---|---|---|---|---|---|
| | used amount | First | Second | stability of first | stability of second |
| Substrate No. | (% by mass) | component | component | component | component |
| Before | 0% | 96.49% | 3.51% | — | — |
| deposition | | | | | |
| (mixed powder) | | | | | |
| No. 1 | 0 to 34% | 99.18% | 0.82%(0.23) | 100.0% | 100.0% |
| No. 2 | 34 to 51% | 99.21% | 0.79%(0.22) | 100.0% | 96.5% |
| No. 3 | 51 to 68% | 99.19% | 0.81%(0.23) | 100.0% | 99.1% |
| No. 4 | 68 to 86% | 98.92% | 1.08%(0.31) | 99.7% | 131.7% |

### Example 2-1

The compounds shown in Table 5 were mixed in the amounts shown in Table 5 to obtain mixed powders. T1, T2, and the value “T1−T2” in the Example are shown in Table 5. Further, the deposition characteristic curves of temperature and P/M^{1/2} of BH-2 and BD-2 are shown in FIG. 4.

TABLE 5

| First component | BH-2 | |
|---|---|---|
| Second component | BD-2 | |
| MOL1 | 99.13 | mol % |
| MOL2 | 0.87 | mol % |
| M1 | 554.69 | |
| M2 | 1003.31 | |
| T1 | 349° | C. |
| T2 | 339° | C. |
| T1 − T2 | 10° | C. |

The continuous deposition was conducted and evaluated using the obtained powder in the same manner as in Example 1-1. The results are shown in Table 6. The total film thickness deposited on the substrates No. 1 to No. 4 was 503 nm.

TABLE 6

| | Cumulative | Molar ratio | | Deposition ratio | Deposition ratio |
|---|---|---|---|---|---|
| | used amount | First | Second | stability of first | stability of second |
| Substrate No. | (% by mass) | component | component | component | component |
| Before | 0% | 99.13% | 0.87% | — | — |
| deposition | | | | | |
| (mixed powder) | | | | | |
| No. 1 | 0 to 25% | 99.87% | 0.13%(0.15) | 100.0% | 100.0% |
| No. 2 | 25 to 37% | 99.84% | 0.16%(0.18) | 100.0% | 126.1% |
| No. 3 | 37 to 49% | 99.86% | 0.14%(0.16) | 100.0% | 108.7% |
| No. 4 | 49 to 62% | 99.85% | 0.15%(0.17) | 100.0% | 117.4% |

### Example 2-2

The same compounds as those used in Example 2-1 were mixed in the amounts shown in Table 7 to obtain mixed powders. T1, T2, and the value “T1−T2” in the Example are shown in Table 7.

TABLE 7

| First component | BH-2 | |
|---|---|---|
| Second component | BD-2 | |
| MOL1 | 97.36 | mol % |
| MOL2 | 2.64 | mol % |
| M1 | 554.69 | |
| M2 | 1003.31 | |
| T1 | 349° | C. |
| T2 | 356° | C. |
| T1 − T2 | −7° | C. |

The continuous deposition was conducted and evaluated using the obtained powder in the same manner as in Example 1-1. The results are shown in Table 8. The total film thickness deposited on the substrates No. 1 to No. 4 was 511 nm.

TABLE 8

| | Cumulative | Molar ratio | | Deposition ratio | Deposition ratio |
|---|---|---|---|---|---|
| | used amount | First | Second | stability of first | stability of second |
| Substrate No. | (% by mass) | component | component | component | component |
| Before | 0% | 97.36% | 2.64% | — | — |
| deposition | | | | | |
| (mixed powder) | | | | | |
| No. 1 | 0 to 27% | 99.76% | 0.24%(0.09) | 100.0% | 100.0% |
| No. 2 | 27 to 41% | 99.77% | 0.23%(0.09) | 100.0% | 97.7% |
| No. 3 | 41 to 55% | 99.77% | 0.23%(0.09) | 100.0% | 97.7% |
| No. 4 | 55 to 68% | 99.72% | 0.28%(0.11) | 100.0% | 118.6% |

### Example 3

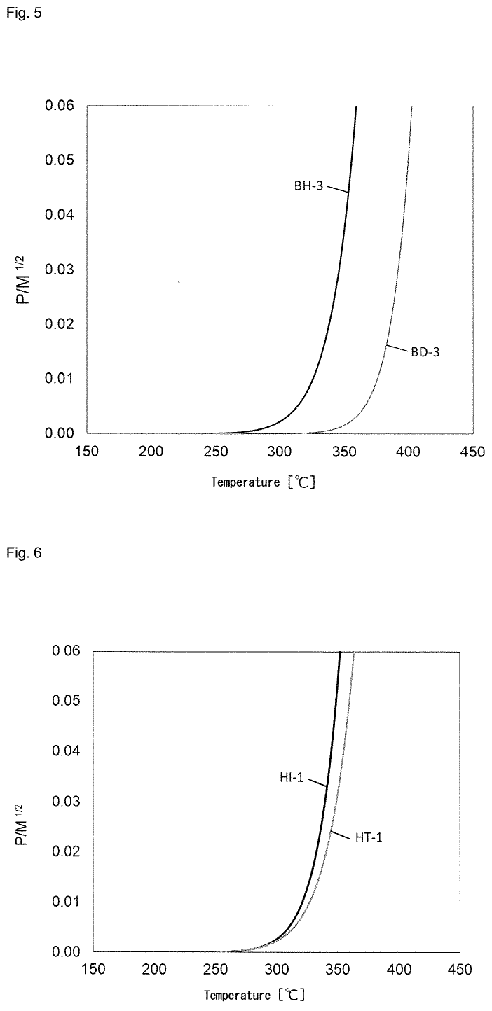

The compounds shown in Table 9 were mixed in the amounts shown in Table 9 to obtain mixed powders. T1, T2, and the value “T1−T2” in the Example are shown in Table 9. Further, the deposition characteristic curves of temperature and P/M^{1/2} of BH-3 and BD-3 are shown in FIG. 5.

TABLE 9

| First component | BH-3 | |
|---|---|---|
| Second component | BD-3 | |
| MOL1 | 98.69 | mol % |
| MOL2 | 1.31 | mol % |
| M1 | 570.69 | |
| M2 | 823.06 | |
| T1 | 351° | C. |
| T2 | 335° | C. |
| T1 − T2 | 16° | C. |

The continuous deposition was conducted and evaluated using the obtained powder in the same manner as in Example 1-1. The results are shown in Table 10. The total film thickness deposited on the substrates No. 1 to No. 5 was 579 nm.

TABLE 10

| | Cumulative | Molar ratio | | Deposition ratio | Deposition ratio |
|---|---|---|---|---|---|
| | used amount | First | Second | stability of first | stability of second |
| Substrate No. | (% by mass) | component | component | component | component |
| Before | 0% | 98.69% | 1.31% | — | — |
| deposition | | | | | |
| (mixed powder) | | | | | |
| No. 1 | 0 to 11% | 99.85% | 0.15%(0.12) | 100.0% | 100.0% |
| No. 2 | 11 to 22% | 99.72% | 0.28%(0.22) | 99.9% | 183.5% |
| No. 3 | 22 to 33% | 99.69% | 0.31%(0.24) | 99.8% | 202.2% |
| No. 4 | 33 to 44% | 99.60% | 0.40%(0.31) | 99.8% | 260.0% |
| No. 5 | 44 to 55% | 99.49% | 0.51%(0.39) | 99.6% | 332.9% |

### Comparative Example 1

The compounds shown in Table 11 were mixed in the amounts shown in Table 11 to obtain mixed powders. T1, T2, and the value “T1−T2” in the Example are shown in Table 11. Further, the deposition characteristic curves of temperature and P/M^{1/2} of HI-1 and HT-1 are shown in FIG. 6.

TABLE 11

| First component | HI-1 | |
|---|---|---|
| Second component | HT-1 | |
| MOL1 | 97.23 | mol % |
| MOL2 | 2.77 | mol % |
| M1 | 802.58 | |
| M2 | 690.89 | |
| T1 | 344° | C. |
| T2 | 287° | C. |
| T1 − T2 | 57° | C. |

The continuous deposition was conducted and evaluated using the obtained powder in the same manner as in Example 1-1. The results are shown in Table 12. The total film thickness deposited on the substrates No. 1 to No. 5 was 467 nm.

TABLE 12

| | Cumulative | Molar ratio | | Deposition ratio | Deposition ratio |
|---|---|---|---|---|---|
| | used amount | First | Second | stability of first | stability of second |
| Substrate No. | (% by mass) | component | component | component | component |
| Before | 0% | 97.23% | 2.77% | — | — |
| deposition | | | | | |
| (mixed powder) | | | | | |
| No. 1 | 0 to 16% | 86.51% | 13.49%(4.88) | 100.0% | 100.0% |
| No. 2 | 16 to 30% | 90.79% | 9.21%(3.33) | 105.0% | 68.3% |
| No. 3 | 30 to 45% | 96.96% | 3.04%(1.10) | 112.1% | 22.5% |
| No. 4 | 45 to 61% | 99.96% | 0.04%(0.02) | 115.5% | 0.3% |
| No. 5 | 61 to 76% | 99.98% | 0.02%(0.01) | 115.6% | 0.1% |

As soon from Examples 1-1,1-2, 2-1, 2-2 and 3, it was found that the component ratio in the deposited film on each substrate was not varied greatly to achieve the deposition with stable component ratio over a long period of time. On the other hand, it was found that in Comparative Example 1, most of the second component was vaporized at the initial stage of the continuous deposition process, and therefore, the difference in (the variability of) the component ratio on each substrate was increased. When the result is applied to actual fabrication of organic EL elements, only a few deposited films which can be used for devices were obtained and as a result, the yield thereof was extremely decreased.

### Definition

In this specification, a hydrogen atom includes its isotopes different in the number of neutrons, namely, a protium, a deuterium and a tritium.

In this specification, at a bondable position in a chemical formula where a symbol such as “R”, or “D” representing a deuterium atom is not indicated, a hydrogen atom, that is, a protium atom, a deuterium atom or a tritium atom is bonded.

In this specification, the number of ring carbon atoms represents the number of carbon atoms forming a subject ring itself among the carbon atoms of a compound having a structure in which atoms are bonded in a ring form (for example, a monocyclic compound, a fused ring compound, a cross-linked compound, a carbocyclic compound, or a heterocyclic compound). When the subject ring is substituted by a substituent, the carbon contained in the substituent is not included in the number of ring carbon atoms. The same shall apply to “the number of ring carbon atoms” described below, unless otherwise specified. For example, a benzene ring has 6 ring carbon atoms, a naphthalene ring includes 10 ring carbon atoms, a pyridine ring includes 5 ring carbon atoms, and a furan ring includes 4 ring carbon atoms. Further, for example, a 9,9-diphenylfluorenyl group includes 13 ring carbon atoms, and a 9,9′-spirobifluorenyl group includes 25 ring carbon atoms.

When a benzene ring is substituted by, for example, an alkyl group as a substituent, the number of carbon atoms of the alkyl group is not included in the number of ring carbon atoms of the benzene ring. Therefore, the number of ring carbon atoms of the benzene ring substituted by the alkyl group is 6. When a naphthalene ring is substituted by, for example, an alkyl group as a substituent, the number of carbon atoms of the alkyl group is not included in the number of ring carbon atoms of the naphthalene ring. Therefore, the number of ring carbon atoms of the naphthalene ring substituted by the alkyl group is 10.

In this specification, the number of ring atoms represents the number of atoms forming a subject ring itself among the atoms of a compound having a structure in which atoms are bonded in a ring form (for example, the structure includes a monocyclic ring, a fused ring and a ring assembly) (for example, a monocyclic compound, a fused ring compound, a cross-linked compound, a carbocyclic compound and a heterocyclic compound). The number of ring atoms does not include atoms which do not form the ring (for example, a hydrogen atom which terminates a bond of the atoms forming the ring), or atoms contained in a substituent when the ring is substituted by the substituent. The same shall apply to “the number of ring atoms” described below, unless otherwise specified. For example, the number of atoms of a pyridine ring is 6, the number of atoms of a quinazoline ring is 10, and the number of a furan ring is 5. For example, hydrogen atoms bonded to a pyridine ring and atoms constituting a substituent substituted on the pyridine ring are not included in the number of ring atoms of the pyridine ring. Therefore, the number of ring atoms of a pyridine ring with which a hydrogen atom or a substituent is bonded is 6. For example, hydrogen atoms and atoms constituting a substituent which are bonded with a quinazoline ring is not included in the number of ring atoms of the quinazoline ring. Therefore, the number of ring atoms of a quinazoline ring with which a hydrogen atom or a substituent is bonded is 10.

In this specification, “XX to YY carbon atoms” in the expression “a substituted or unsubstituted ZZ group including XX to YY carbon atoms” represents the number of carbon atoms in the case where the ZZ group is unsubstituted by a substituent, and does not include the number of carbon atoms of a substituent in the case where the ZZ group is substituted by the substituent. Here, “YY” is larger than “XX”, and “XX” means an integer of 1 or more and “YY” means an integer of 2 or more.

In this specification, “XX to YY atoms” in the expression “a substituted or unsubstituted ZZ group including XX to YY atoms” represents the number of atoms in the case where the ZZ group is unsubstituted by a substituent, and does not include the number of atoms of a substituent in the case where the ZZ group is substituted by the substituent. Here, “YY” is larger than “XX”, and “XX” means an integer of 1 or more and “YY” means an integer of 2 or more.

In this specification, the unsubstituted ZZ group represents the case where the “substituted or unsubstituted ZZ group” is a “ZZ group unsubstituted by a substituent”, and the substituted ZZ group represents the case where the “substituted or unsubstituted ZZ group” is a “ZZ group substituted by a substituent”.

In this specification, a term “unsubstituted” in the case of “a substituted or unsubstituted ZZ group” means that hydrogen atoms in the ZZ group are not substituted by a substituent. Hydrogen atoms in a term “unsubstituted ZZ group” are a protium atom, a deuterium atom, or a tritium atom.

In this specification, a term “substituted” in the case of “a substituted or unsubstituted ZZ group” means that one or more hydrogen atoms in the ZZ group are substituted by a substituent. Similarly, a term “substituted” in the case of “a BB group substituted by an AA group” means that one or more hydrogen atoms in the BB group are substituted by the AA group.

“Substituent as Described in this Specification”

Hereinafter, the substituent described in this specification will be explained.

The number of ring carbon atoms of the “unsubstituted aryl group” described in this specification is 6 to 50, preferably 6 to 30, and more preferably 6 to 18, unless otherwise specified.

The number of ring atoms of the “unsubstituted heterocyclic group” described in this specification is 5 to 50, preferably 5 to 30, and more preferably 5 to 18, unless otherwise specified.

The number of carbon atoms of the “unsubstituted alkyl group” described in this specification is 1 to 50, preferably 1 to 20, and more preferably 1 to 6, unless otherwise specified.

The number of carbon atoms of the “unsubstituted alkenyl group” described in this specification is 2 to 50, preferably 2 to 20, and more preferably 2 to 6, unless otherwise specified.

The number of carbon atoms of the “unsubstituted alkynyl group” described in this specification is 2 to 50, preferably 2 to 20, and more preferably 2 to 6, unless otherwise specified.

The number of ring carbon atoms of the “unsubstituted cycloalkyl group” described in this specification is 3 to 50, preferably 3 to 20, and more preferably 3 to 6, unless otherwise specified.

The number of ring carbon atoms of the “unsubstituted arylene group” described in this specification is 6 to 50, preferably 6 to 30, and more preferably 6 to 18, unless otherwise specified.

The number of ring atoms of the “unsubstituted divalent heterocyclic group” described in this specification is 5 to 50, preferably 5 to 30, and more preferably 5 to 18, unless otherwise specified.

The number of carbon atoms of the “unsubstituted alkylene group” described in this specification is 1 to 50, preferably 1 to 20, and more preferably 1 to 6, unless otherwise specified.

### · “Substituted or Unsubstituted Aryl Group”

Specific examples of the “substituted or unsubstituted aryl group” described in this specification (specific example group G1) include the following unsubstituted aryl groups (specific example group G1A), substituted aryl groups (specific example group G1B), and the like. (Here, the unsubstituted aryl group refers to the case where the “substituted or unsubstituted aryl group” is an “aryl group unsubstituted by a substituent”, and the substituted aryl group refers to the case where the “substituted or unsubstituted aryl group” is an “aryl group substituted by a substituent”). In this specification, in the case where simply referred as an “aryl group”, it includes both a “unsubstituted aryl group” and a “substituted aryl group.”

The “substituted aryl group” means a group in which one or more hydrogen atoms of the “unsubstituted aryl group” are substituted by a substituent. Specific examples of the “substituted aryl group” include, for example, groups in which one or more hydrogen atoms of the “unsubstituted aryl group” of the following specific example group G1A are substituted by a substituent, the substituted aryl groups of the following specific example group G1B, and the like. It should be noted that the examples of the “unsubstituted aryl group” and the examples of the “substituted aryl group” enumerated in this specification are mere examples, and the “substituted aryl group” described in this specification also includes a group in which a hydrogen atom bonded with a carbon atom of the aryl group itself in the “substituted aryl group” of the following specific group G1B is further substituted by a substituent, and a group in which a hydrogen atom of a substituent in the “substituted aryl group” of the following specific group G1B is further substituted by a substituent.

### · Unsubstituted Aryl Group (Specific Example Group G1A):

a phenyl group,
a p-biphenyl group,
a m-biphenyl group,
an o-biphenyl group,
a p-terphenyl-4-yl group,
a p-terphenyl-3-yl group,
a p-terphenyl-2-yl group,
a m-terphenyl-4-yl group,
a m-terphenyl-3-yl group,
a m-terphenyl-2-yl group,
an o-terphenyl-4-yl group,
an o-terphenyl-3-yl group,
an o-terphenyl-2-yl group,
a 1-naphthyl group,
a 2-naphthyl group,
an anthryl group,
a benzanthryl group,
a phenanthryl group,
a benzophenanthryl group,
a phenalenyl group,
a pyrenyl group,
a chrysenyl group,
a benzochrysenyl group,
a triphenylenyl group,
a benzotriphenylenyl group,
a tetracenyl group,
a pentacenyl group,
a fluorenyl group,
a 9,9′-spirobifluorenyl group,
a benzofluorenyl group,
a dibenzofluorenyl group,
a fluoranthenyl group,
a benzofluoranthenyl group,
a perylenyl group, and
a monovalent aryl group derived by removing one hydrogen atom from the ring structures represented by any of the following general formulas (TEMP-1) to (TEMP-15).

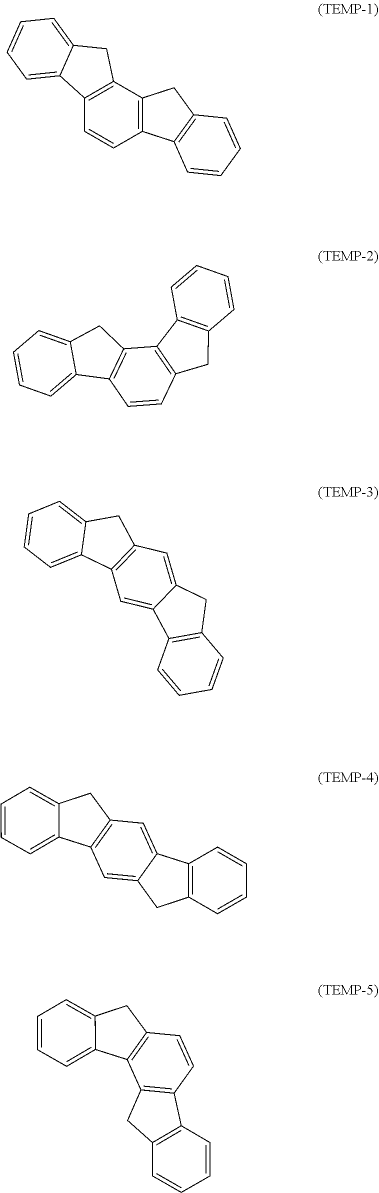

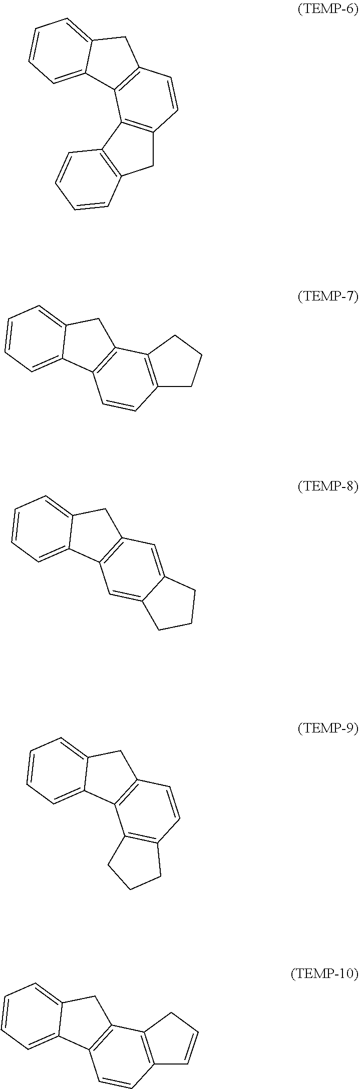

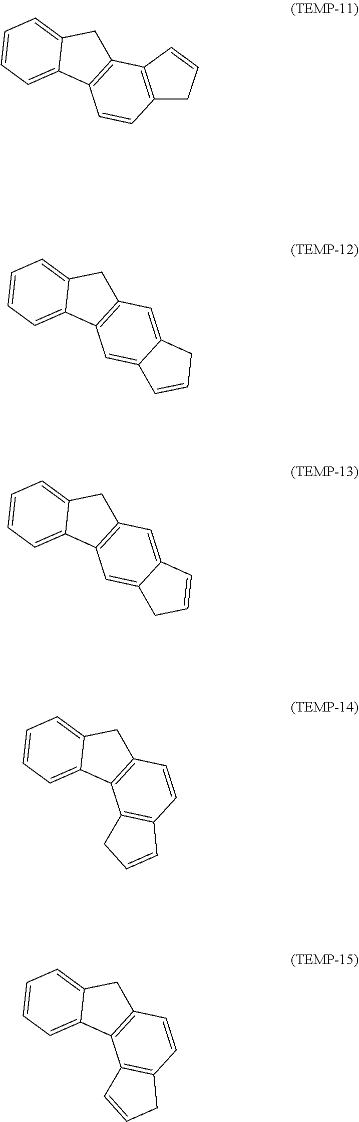

### · Substituted Aryl Group (Specific Example Group G1B):

an o-tolyl group,
a m-tolyl group,
a p-tolyl group,
a p-xylyl group,
a m-xylyl group,
an o-xylyl group,
a p-isopropylphenyl group,
a m-isopropylphenyl group,
an o-isopropylphenyl group,
a p-t-butylphenyl group,
a m-t-butylphenyl group,
an o-t-butylphenyl group,
a 3,4,5-trimethylphenyl group,
a 9,9-dimethylfluorenyl group,
a 9,9-diphenylfluorenyl group,
a 9,9-bis(4-methylphenyl)fluorenyl group,
a 9,9-bis(4-isopropylphenyl)fluorenyl group,
a 9,9-bis(4-t-butylphenyl)fluorenyl group,
a cyanophenyl group,
a triphenylsilylphenyl group,
a trimethylsilylphenyl group,
a phenylnaphthyl group,
a naphthylphenyl group, and
a group in which one or more hydrogen atoms of a monovalent group derived from the ring structures represented by any of the general formulas (TEMP-1) to (TEMP-15) are substituted by a substituent.

### · “Substituted or Unsubstituted Heterocyclic Group”

The “heterocyclic group” described in this specification is a ring group having at least one hetero atom in the ring atom. Specific examples of the hetero atom include a nitrogen atom, an oxygen atom, a sulfur atom, a silicon atom, a phosphorus atom, and a boron atom.

The “heterocyclic group” in this specification is a monocyclic group or a fused ring group.

The “heterocyclic group” in this specification is an aromatic heterocyclic group or a non-aromatic heterocyclic group.

Specific examples of the “substituted or unsubstituted heterocyclic group” (specific example group G2) described in this specification include the following unsubstituted heterocyclic group (specific example group G2A), the following substituted heterocyclic group (specific example group G2B), and the like. (Here, the unsubstituted heterocyclic group refers to the case where the “substituted or unsubstituted heterocyclic group” is a “heterocyclic group unsubstituted by a substituent”, and the substituted heterocyclic group refers to the case where the “substituted or unsubstituted heterocyclic group” is a “heterocyclic group substituted by a substituent”). In this specification, in the case where simply referred as a “heterocyclic group”, it includes both the “unsubstituted heterocyclic group” and the “substituted heterocyclic group.”

The “substituted heterocyclic group” means a group in which one or more hydrogen atom of the “unsubstituted heterocyclic group” are substituted by a substituent. Specific examples of the “substituted heterocyclic group” include a group in which a hydrogen atom of “unsubstituted heterocyclic group” of the following specific example group G2A is substituted by a substituent, the substituted heterocyclic groups of the following specific example group G2B, and the like. It should be noted that the examples of the “unsubstituted heterocyclic group” and the examples of the “substituted heterocyclic group” enumerated in this specification are mere examples, and the “substituted heterocyclic group” described in this specification includes groups in which hydrogen atom bonded with a ring atom of the heterocyclic group itself in the “substituted heterocyclic group” of the specific example group G2B is further substituted by a substituent, and a group in which hydrogen atom of a substituent in the “substituted heterocyclic group” of the specific example group G2B is further substituted by a substituent.

Specific example group G2A includes, for example, the following unsubstituted heterocyclic group containing a nitrogen atom (specific example group G2A1), the following unsubstituted heterocyclic group containing an oxygen atom (specific example group G2A2), the following unsubstituted heterocyclic group containing a sulfur atom (specific example group G2A3), and the monovalent heterocyclic group derived by removing one hydrogen atom from the ring structures represented by any of the following general formulas (TEMP-16) to (TEMP-33) (specific example group G2A4).

Specific example group G2B includes, for example, the following substituted heterocyclic group containing a nitrogen atom (specific example group G2B1), the following substituted heterocyclic group containing an oxygen atom (specific example group G2B2), the following substituted heterocyclic group containing a sulfur atom (specific example group G2B3), and the following group in which one or more hydrogen atoms of the monovalent heterocyclic group derived from the ring structures represented by any of the following general formulas (TEMP-16) to (TEMP-33) are substituted by a substituent (specific example group G2B4).

### · Unsubstituted Heterocyclic Group Containing a Nitrogen Atom (Specific Example Group G2A1):

a pyrrolyl group,
an imidazolyl group,
a pyrazolyl group,
a triazolyl group,
a tetrazolyl group,
an oxazolyl group,
an isoxazolyl group,
an oxadiazolyl group,
a thiazolyl group,
an isothiazolyl group,
a thiadiazolyl group,
a pyridyl group,
a pyridazinyl group,
a pyrimidinyl group,
a pyrazinyl group,
a triazinyl group,
an indolyl group,
an isoindolyl group,
an indolizinyl group,
a quinolizinyl group,
a quinolyl group,
an isoquinolyl group,
a cinnolyl group,
a phthalazinyl group,
a quinazolinyl group,
a quinoxalinyl group,
a benzimidazolyl group,
an indazolyl group,
a phenanthrolinyl group,
a phenanthridinyl group,
an acridinyl group,
a phenazinyl group,
a carbazolyl group,
a benzocarbazolyl group,
a morpholino group,
a phenoxazinyl group,
a phenothiazinyl group,
an azacarbazolyl group, and
a diazacarbazolyl group.

### · Unsubstituted Heterocyclic Group Containing an Oxygen Atom (Specific Example Group G2A2):

a furyl group,
an oxazolyl group,
an isoxazolyl group,
an oxadiazolyl group,
a xanthenyl group,
a benzofuranyl group,
an isobenzofuranyl group,
a dibenzofuranyl group,
a naphthobenzofuranyl group,
a benzoxazolyl group,
a benzisoxazolyl group,
a phenoxazinyl group,
a morpholino group,
a dinaphthofuranyl group,
an azadibenzofuranyl group,
a diazadibenzofuranyl group,
an azanaphthobenzofuranyl group, and
a diazanaphthobenzofuranyl group.

### · Unsubstituted Heterocyclic Group Containing a Sulfur Atom (Specific Example Group G2A3):

a thienyl group,
a thiazolyl group,
an isothiazolyl group,
a thiadiazolyl group,
a benzothiophenyl group (benzothienyl group),
an isobenzothiophenyl group (isobenzothienyl group),
a dibenzothiophenyl group (dibenzothienyl group),
a naphthobenzothiophenyl group (naphthobenzothienyl group),
a benzothiazolyl group,
a benzisothiazolyl group,
a phenothiazinyl group,
a dinaphthothiophenyl group (dinaphthothienyl group),
an azadibenzothiophenyl group (azadibenzothienyl group),
a diazadibenzothiophenyl group (diazadibenzothienyl group),
an azanaphthobenzothiophenyl group (azanaphthobenzothienyl group), and
a diazanaphthobenzothiophenyl group (diazanaphthobenzothienyl group).
   · Monovalent Heterocyclic Group Derived by Removing One Hydrogen Atom from the Ring Structures Represented by any of the Following General Formulas (TEMP-16) to (TEMP-33) (Specific Example Group G2A4):

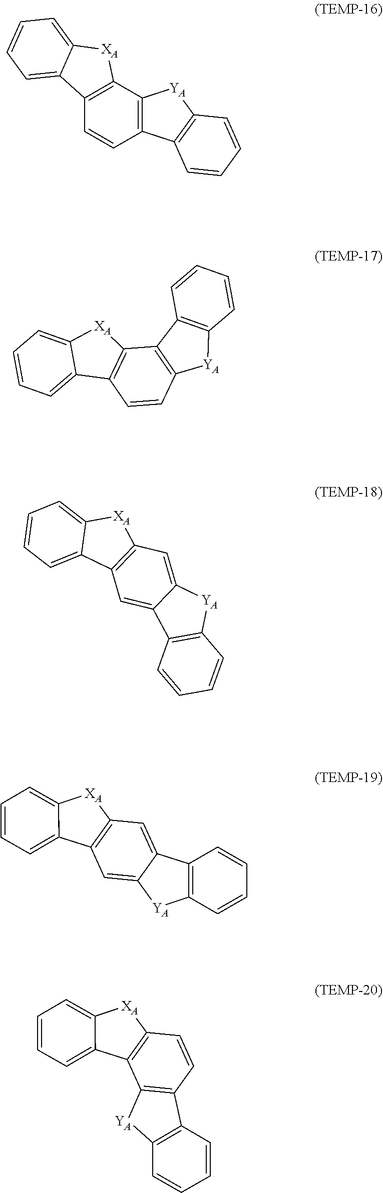

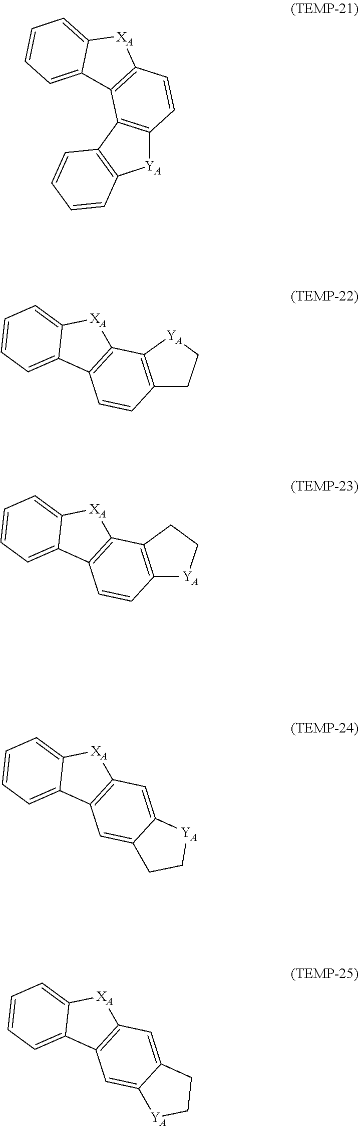

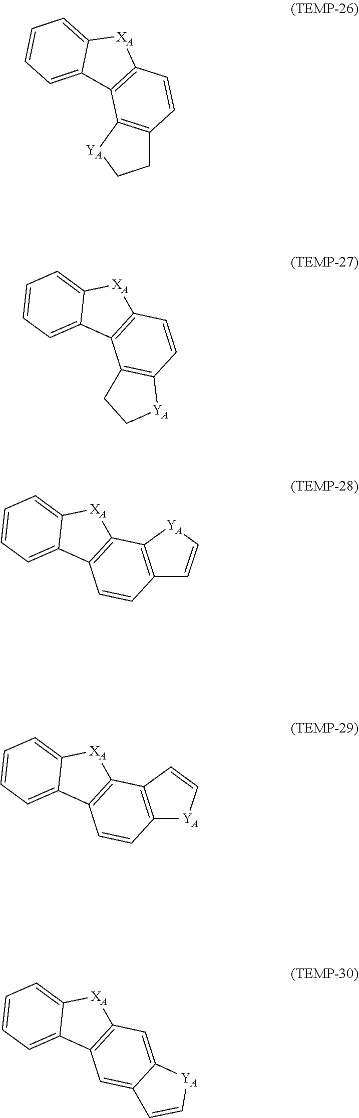

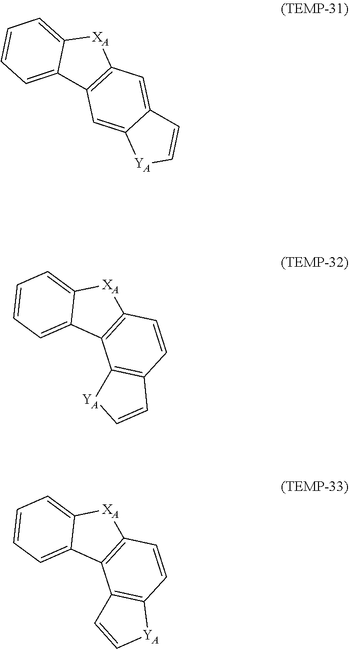

In the general formulas (TEMP-16) to (TEMP-33), XA and YA are independently an oxygen atom, a sulfur atom, NH, or CH₂. Provided that at least one of XA and YA is an oxygen atom, a sulfur atom, or NH.

In the general formulas (TEMP-16) to (TEMP-33), when at least one of XA and YA is NH or CH₂, the monovalent heterocyclic group derived from the ring structures represented by any of the general formulas (TEMP-16) to (TEMP-33) includes a monovalent group derived by removing one hydrogen atom from these NH or CH₂.

### · Substituted Heterocyclic Group Containing a Nitrogen Atom (Specific Example Group G2B1):

a (9-phenyl)carbazolyl group,
a (9-biphenylyl)carbazolyl group,
a (9-phenyl)phenylcarbazolyl group,
a (9-naphthyl)carbazolyl group,
a diphenylcarbazol-9-yl group,
a phenylcarbazol-9-yl group,
a methylbenzimidazolyl group,
an ethylbenzimidazolyl group,
a phenyltriazinyl group,
a biphenylyltriazinyl group,
a diphenyltriazinyl group,
a phenylquinazolinyl group, and
a biphenylylquinazolinyl group.

### · Substituted Heterocyclic Group Containing an Oxygen Atom (Specific Example Group G2B2):

a phenyldibenzofuranyl group,
a methyldibenzofuranyl group,
a t-butyldibenzofuranyl group, and
a monovalent residue of spiro[9H-xanthene-9,9′-[9H]fluorene].

### · Substituted Heterocyclic Group Containing a Sulfur Atom (Specific Example Group G2B3):

a phenyldibenzothiophenyl group,
a methyldibenzothiophenyl group,
a t-butyldibenzothiophenyl group, and
a monovalent residue of spiro[9H-thioxanthene-9,9′-[9H]fluorene].
   · Group in which One or More Hydrogen Atoms of the Monovalent Heterocyclic Group Derived from the Ring Structures Represented by any of the Following General Formulas (TEMP-16) to (TEMP-33) are Substituted by a Substituent (Specific Example Group G2B4):

The “one or more hydrogen atoms of the monovalent heterocyclic group” means one or more hydrogen atoms selected from hydrogen atoms bonded with ring carbon atoms of the monovalent heterocyclic group, a hydrogen atom bonded with a nitrogen atom when at least one of XA and YA is NH, and hydrogen atoms of a methylene group when one of XA and YA is CH₂.

### · “Substituted or Unsubstituted Alkyl Group”

Specific examples of the “substituted or unsubstituted alkyl group” (specific example group G3) described in this specification include the following unsubstituted alkyl groups (specific example group G3A) and the following substituted alkyl groups (specific example group G3B). (Here, the unsubstituted alkyl group refers to the case where the “substituted or unsubstituted alkyl group” is an “alkyl group unsubstituted by a substituent”, and the substituted alkyl group refers to the case where the “substituted or unsubstituted alkyl group” is an “alkyl group substituted by a substituent”). In this specification, in the case where simply referred as an “alkyl group” includes both the “unsubstituted alkyl group” and the “substituted alkyl group.”

The “substituted alkyl group” means a group in which one or more hydrogen atoms in the “unsubstituted alkyl group” are substituted by a substituent. Specific examples of the “substituted alkyl group” include groups in which one or more hydrogen atoms in the following “unsubstituted alkyl group” (specific example group G3A) are substituted by a substituent, the following substituted alkyl group (specific example group G3B), and the like. In this specification, the alkyl group in the “unsubstituted alkyl group” means a linear alkyl group. Thus, the “unsubstituted alkyl group” includes a straight-chain “unsubstituted alkyl group” and a branched-chain “unsubstituted alkyl group”. It should be noted that the examples of the “unsubstituted alkyl group” and the examples of the “substituted alkyl group” enumerated in this specification are mere examples, and the “substituted alkyl group” described in this specification includes a group in which hydrogen atom of the alkyl group itself in the “substituted alkyl group” of the specific example group G3B is further substituted by a substituent, and a group in which hydrogen atom of a substituent in the “substituted alkyl group” of the specific example group G3B is further substituted by a substituent.

### · Unsubstituted Alkyl Group (Specific Example Group G3A):

a methyl group,
an ethyl group,
a n-propyl group,
an isopropyl group,
a n-butyl group,
an isobutyl group,
a s-butyl group, and
a t-butyl group.

### · Substituted Alkyl Group (Specific Example Group G3B):

a heptafluoropropyl group (including isomers),
a pentafluoroethyl group,
a 2,2,2-trifluoroethyl group, and
a trifluoromethyl group.

### · “Substituted or Unsubstituted Alkenyl Group”

Specific examples of the “substituted or unsubstituted alkenyl group” described in this specification (specific example group G4) include the following unsubstituted alkenyl group (specific example group G4A), the following substituted alkenyl group (specific example group G4B), and the like. (Here, the unsubstituted alkenyl group refers to the case where the “substituted or unsubstituted alkenyl group” is a “alkenyl group unsubstituted by a substituent”, and the “substituted alkenyl group” refers to the case where the “substituted or unsubstituted alkenyl group” is a “alkenyl group substituted by a substituent.”). In this specification, in the case where simply referred as an “alkenyl group” includes both the “unsubstituted alkenyl group” and the “substituted alkenyl group.”

The “substituted alkenyl group” means a group in which one or more hydrogen atoms in the “unsubstituted alkenyl group” are substituted by a substituent. Specific examples of the “substituted alkenyl group” include a group in which the following “unsubstituted alkenyl group” (specific example group G4A) has a substituent, the following substituted alkenyl group (specific example group G4B), and the like. It should be noted that the examples of the “unsubstituted alkenyl group” and the examples of the “substituted alkenyl group” enumerated in this specification are mere examples, and the “substituted alkenyl group” described in this specification includes a group in which a hydrogen atom of the alkenyl group itself in the “substituted alkenyl group” of the specific example group G4B is further substituted by a substituent, and a group in which a hydrogen atom of a substituent in the “substituted alkenyl group” of the specific example group G4B is further substituted by a substituent.

### · Unsubstituted Alkenyl Group (Specific Example Group G4A):

a vinyl group,
an allyl group,
a 1-butenyl group,
a 2-butenyl group, and
a 3-butenyl group.

### · Substituted Alkenyl Group (Specific Example Group G4B):

a 1,3-butanedienyl group,
a 1-methylvinyl group,
a 1-methylallyl group,
a 1,1-dimethylallyl group,
a 2-methylally group, and
a 1,2-dimethylallyl group.

### · “Substituted or Unsubstituted Alkynyl Group”

Specific examples of the “substituted or unsubstituted alkynyl group” described in this specification (specific example group G5) include the following unsubstituted alkynyl group (specific example group G5A) and the like. (Here, the unsubstituted alkynyl group refers to the case where the “substituted or unsubstituted alkynyl group” is an “alkynyl group unsubstituted by a substituent”). In this specification, in the case where simply referred as an “alkynyl group” includes both the “unsubstituted alkynyl group” and the “substituted alkynyl group.”

The “substituted alkynyl group” means a group in which one or more hydrogen atoms in the “unsubstituted alkynyl group” are substituted by a substituent. Specific examples of the “substituted alkynyl group” include a group in which one or more hydrogen atoms in the following “unsubstituted alkynyl group” (specific example group G5A) are substituted by a substituent, and the like.

### · Unsubstituted Alkynyl Group (Specific Example Group G5A):

an ethynyl group.

### · “Substituted or Unsubstituted Cycloalkyl Group”

Specific examples of the “substituted or unsubstituted cycloalkyl group” described in this specification (specific example group G6) include the following unsubstituted cycloalkyl group (specific example group G6A), the following substituted cycloalkyl group (specific example group G6B), and the like. (Here, the unsubstituted cycloalkyl group refers to the case where the “substituted or unsubstituted cycloalkyl group” is a “cycloalkyl group unsubstituted by a substituent”, and the substituted cycloalkyl group refers to the case where the “substituted or unsubstituted cycloalkyl group” is a “cycloalkyl group substituted by a substituent”). In this specification, in the case where simply referred as a “cycloalkyl group” includes both the “unsubstituted cycloalkyl group” and the “substituted cycloalkyl group.”

The “substituted cycloalkyl group” means a group in which one or more hydrogen atoms in the “unsubstituted cycloalkyl group” are substituted by a substituent. Specific examples of the “substituted cycloalkyl group” include a group in which one or more hydrogen atoms in the following “unsubstituted cycloalkyl group” (specific example group G6A) are substituted by a substituent, and examples of the following substituted cycloalkyl group (specific example group G6B), and the like. It should be noted that the examples of the “unsubstituted cycloalkyl group” and the examples of the “substituted cycloalkyl group” enumerated in this specification are mere examples, and the “substituted cycloalkyl group” in this specification includes a group in which one or more hydrogen atoms bonded with the carbon atom of the cycloalkyl group itself in the “substituted cycloalkyl group” of the specific example group G6B are substituted by a substituent, and a group in which a hydrogen atom of a substituent in the “substituted cycloalkyl group” of specific example group G6B is further substituted by a substituent.

### · Unsubstituted Cycloalkyl Group (Specific Example Group G6A):

a cyclopropyl group,
a cyclobutyl group,
a cyclopentyl group,
a cyclohexyl group,
a 1-adamantyl group,
a 2-adamantyl group,
a 1-norbornyl group, and
a 2-norbornyl group.

### · Substituted Cycloalkyl Group (Specific Example Group G6B):

a 4-methylcyclohexyl group.

· “Group Represented by —Si (R₉₀₁)(R₉₀₂)(R₉₀₃)”

Specific examples of the group represented by —Si(R₉₀₁) (R₉₀₂)(R₉₀₃) described in this specification (specific example group G7) include:
—Si(G1)(G1)(G1),
—Si(G1)(G2)(G2),
—Si(G1)(G1)(G2),
—Si(G2)(G2)(G2),
—Si(G3)(G3)(G3), and
—Si(G6)(G6)(G6).

G1 is the “substituted or unsubstituted aryl group” described in the specific example group G1.

G2 is the “substituted or unsubstituted heterocyclic group” described in the specific example group G2.

G3 is the “substituted or unsubstituted alkyl group” described in the specific example group G3.

G6 is the “substituted or unsubstituted cycloalkyl group” described in the specific example group G6.

Plural G1's in —Si(G1)(G1)(G1) are the same or different.

Plural G2's in —Si(G1)(G2)(G2) are the same or different.

Plural G1's in —Si(G1)(G1)(G2) are the same or different.

Plural G2's in —Si(G2)(G2)(G2) are be the same or different.

Plural G3's in —Si(G3)(G3)(G3) are the same or different.

Plural G6's in —Si(G6)(G6)(G6) are be the same or different.

### · “Group Represented by —O—(R₉₀₄)”

Specific examples of the group represented by —O—(R₉₀₄) in this specification (specific example group G8) include:
—O(G1),
—O(G2),
—O(G3), and
—O(G6).

G1 is the “substituted or unsubstituted aryl group” described in the specific example group G1.

G2 is the “substituted or unsubstituted heterocyclic group” described in the specific example group G2.

G3 is the “substituted or unsubstituted alkyl group” described in the specific example group G3.

G6 is the “substituted or unsubstituted cycloalkyl group” described in the specific example group G6.

### · “Group Represented by —S—(R₉₀₅)”

Specific examples of the group represented by —S—(R₉₀₅) in this specification (specific example group G9) include:
—S(G1),
—S(G2),
—S(G3), and
—S(G6).

G1 is the “substituted or unsubstituted aryl group” described in the specific example group G1.

G2 is the “substituted or unsubstituted heterocyclic group” described in the specific example group G2.

G3 is the “substituted or unsubstituted alkyl group” described in the specific example group G3.

G6 is the “substituted or unsubstituted cycloalkyl group” described in the specific example group G6.

· “Group Represented by —N(R₉₀₆) (R₉₀₇)”

Specific examples of the group represented by —N(R₉₀₆) (R₉₀₇) in this specification (specific example group G10) include:
—N(G1)(G1),
—N(G2)(G2),
—N(G1)(G2),
—N(G3)(G3), and
—N(G6)(G6).

G1 is the “substituted or unsubstituted aryl group” described in the specific example group G1.

G2 is the “substituted or unsubstituted heterocyclic group” described in the specific example group G2.

G3 is the “substituted or unsubstituted alkyl group” described in the specific example group G3.

G6 is the “substituted or unsubstituted cycloalkyl group” described in the specific example group G6.

Plural G1's in —N(G1)(G1) are the same or different.

Plural G2's in —N(G2)(G2) are the same or different.

Plural G3's in —N(G3)(G3) are the same or different.

Plural G6's in —N(G6)(G6) are the same or different.

### · “Halogen Atom”

Specific examples of the “halogen atom” described in this specification (specific example group G11) include a fluorine atom, a chlorine atom, a bromine atom, an iodine atom, and the like.

### · “Substituted or Unsubstituted Fluoroalkyl Group”

The “substituted or unsubstituted fluoroalkyl group” described in this specification is a group in which at least one hydrogen atom bonded with a carbon atom constituting the alkyl group in the “substituted or unsubstituted alkyl group” is substituted by a fluorine atom, and includes a group in which all hydrogen atoms bonded with a carbon atom constituting the alkyl group in the “substituted or unsubstituted alkyl group” are substituted by a fluorine atom (a perfluoro group). The number of carbon atoms of the “unsubstituted fluoroalkyl group” is 1 to 50, preferably 1 to 30, more preferably 1 to 18, unless otherwise specified in this specification. The “substituted fluoroalkyl group” means a group in which one or more hydrogen atoms of the “fluoroalkyl group” are substituted by a substituent. The “substituted fluoroalkyl group” described in this specification also includes a group in which one or more hydrogen atoms bonded with a carbon atom of the alkyl chains in the “substituted fluoroalkyl group” are further substituted by a substituent, and a group in which one or more hydrogen atom of a substituent in the “substituted fluoroalkyl group” are further substituted by a substituent. Specific examples of the “unsubstituted fluoroalkyl group” include a group in which one or more hydrogen atoms in the “alkyl group” (specific group G3) are substituted by a fluorine atom, and the like.

### · “Substituted or Unsubstituted Haloalkyl Group”

The “substituted or unsubstituted haloalkyl group” described in this specification is a group in which at least one hydrogen atom bonded with a carbon atom constituting the alkyl group in the “substituted or unsubstituted alkyl group” is substituted by a halogen atom, and also includes a group in which all hydrogen atoms bonded with a carbon atom constituting the alkyl group in the “substituted or unsubstituted alkyl group” are substituted by a halogen atom. The number of carbon atoms of the “unsubstituted haloalkyl group” is 1 to 50, preferably 1 to 30, more preferably 1 to 18, unless otherwise specified in this specification. The “substituted haloalkyl group” means a group in which one or more hydrogen atoms of the “haloalkyl group” are substituted by a substituent. The “substituted haloalkyl group” described in this specification also includes a group in which one or more hydrogen atoms bonded with a carbon atom of the alkyl chain in the “substituted haloalkyl group” are further substituted by a substituent, and a group in which one or more hydrogen atoms of a substituent in the “substituted haloalkyl group” are further substituted by a substituent. Specific examples of the “unsubstituted haloalkyl group” include a group in which one or more hydrogen atoms in the “alkyl group” (specific example group G3) are substituted by a halogen atom, and the like. A haloalkyl group is sometimes referred to as an alkyl halide group.

### · “Substituted or Unsubstituted Alkoxy Group”

Specific examples of the “substituted or unsubstituted alkoxy group” described in this specification include a group represented by —O(G3), wherein G3 is the “substituted or unsubstituted alkyl group” described in the specific example group G3. The number of carbon atoms of the “unsubstituted alkoxy group” is 1 to 50, preferably 1 to 30, more preferably 1 to 18, unless otherwise specified in this specification.

### · “Substituted or Unsubstituted Alkylthio Group”

Specific examples of the “substituted or unsubstituted alkylthio group” described in this specification include a group represented by —S(G3), wherein G3 is the “substituted or unsubstituted alkyl group” described in the specific example group G3. The number of carbon atoms of the “unsubstituted alkylthio group” is 1 to 50, preferably 1 to 30, more preferably 1 to 18, unless otherwise specified in this specification.

### · “Substituted or Unsubstituted Aryloxy Group”

Specific examples of the “substituted or unsubstituted aryloxy group” described in this specification include a group represented by —O(G1), wherein G1 is the “substituted or unsubstituted aryl group” described in the specific example group G1. The number of ring carbon atoms of the “unsubstituted aryloxy group” is 6 to 50, preferably 6 to 30, more preferably 6 to 18, unless otherwise specified in this specification.

### · “Substituted or Unsubstituted Arylthio Group”

Specific examples of the “substituted or unsubstituted arylthio group” described in this specification include a group represented by —S(G1), wherein G1 is a “substituted or unsubstituted aryl group” described in the specific example group G1. The number of ring carbon atoms of the “unsubstituted arylthio group” is 6 to 50, preferably 6 to 30, more preferably 6 to 18, unless otherwise specified in this specification.

### · “Substituted or Unsubstituted Trialkylsilyl Group”

Specific examples of the “trialkylsilyl group” described in this specification include a group represented by —Si(G3)(G3)(G3), where G3 is the “substituted or unsubstituted alkyl group” described in the specific example group G3. Plural G3's in —Si(G3)(G3)(G3) are the same or different. The number of carbon atoms in each alkyl group of the “trialkylsilyl group” is 1 to 50, preferably 1 to 20, more preferably 1 to 6, unless otherwise specified in this specification.

### ·“Substituted or Unsubstituted Aralkyl Group”

Specific examples of the “substituted or unsubstituted aralkyl group” described in this specification is a group represented by -(G3)-(G1), wherein G3 is the “substituted or unsubstituted alkyl group” described in the specific example group G3, and G1 is the “substituted or unsubstituted aryl group” described in the specific example group G1. Therefore, the “aralkyl group” is a group in which a hydrogen atom of the “alkyl group” is substituted by an “aryl group” as a substituent, and is one form of the “substituted alkyl group.” The “unsubstituted aralkyl group” is the “unsubstituted alkyl group” substituted by the “unsubstituted aryl group”, and the number of carbon atoms of the “unsubstituted aralkyl group” is 7 to 50, preferably 7 to 30, more preferably 7 to 18, unless otherwise specified in this specification.

Specific examples of the “substituted or unsubstituted aralkyl group” include a benzyl group, a 1-phenylethyl group, a 2-phenylethyl group, a 1-phenylisopropyl group, a 2-phenylisopropyl group, a phenyl-t-butyl group, an α-naphthylmethyl group, a 1-α-naphthylethyl group, a 2-α-naphthylethyl group, a 1-α-naphthylisopropyl group, a 2-α-naphthylisopropyl group, a β-naphthylmethyl group, a 1-β-naphthylethyl group, a 2-β-naphthylethyl group, a 1-β-naphthylisopropyl group, a 2-β-naphthylisopropyl group, and the like.

Unless otherwise specified in this specification, examples of the substituted or unsubstituted aryl group described in this specification preferably include a phenyl group, a p-biphenyl group, a m-biphenyl group, an o-biphenyl group, a p-terphenyl-4-yl group, a p-terphenyl-3-yl group, a p-terphenyl-2-yl group, a m-terphenyl-4-yl group, a m-terphenyl-3-yl group, a m-terphenyl-2-yl group, an o-terphenyl-4-yl group, an o-terphenyl-3-yl group, an o-terphenyl-2-yl group, a 1-naphthyl group, a 2-naphthyl group, an anthryl group, a phenanthryl group, a pyrenyl group, a chrysenyl group, a triphenylenyl group, a fluorenyl group, a 9,9′-spirobifluorenyl group, 9,9-dimethylfluorenyl group, 9,9-diphenylfluorenyl group, and the like.

Unless otherwise specified in this specification, examples of the substituted or unsubstituted heterocyclic groups described in this specification preferably include a pyridyl group, a pyrimidinyl group, a triazinyl group, a quinolyl group, an isoquinolyl group, a quinazolinyl group, a benzimidazolyl group, a phenanthrolinyl group, a carbazolyl group (a 1-carbazolyl group, a 2-carbazolyl group, a 3-carbazolyl group, a 4-carbazolyl group, or a 9-carbazolyl group), a benzocarbazolyl group, an azacarbazolyl group, a diazacarbazolyl group, a dibenzofuranyl group, a naphthobenzofuranyl group, an azadibenzofuranyl group, a diazadibenzofuranyl group, a dibenzothiophenyl group, a naphthobenzothiophenyl group, an azadibenzothiophenyl group, a diazadibenzothiophenyl group, a (9-phenyl)carbazolyl group (a (9-phenyl)carbazol-1-yl group, a (9-phenyl)carbazol-2-yl group, a (9-phenyl)carbazol-3-yl group, or a (9-phenyl)carbazol-4-yl group), a (9-biphenylyl)carbazolyl group, a (9-phenyl)phenylcarbazolyl group, a diphenylcarbazol-9-yl group, a phenylcarbazol-9-yl group, a phenyltriazinyl group, a biphenylyltriazinyl group, a diphenyltriazinyl group, a phenyldibenzofuranyl group, a phenyldibenzothiophenyl group, and the like.

In this specification, the carbazolyl group is specifically any of the following groups, unless otherwise specified in this specification.

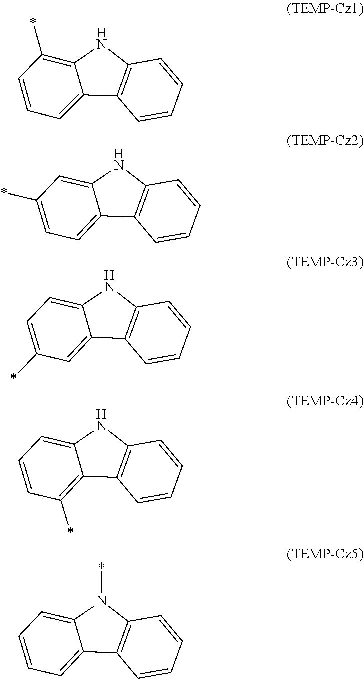 [SMILES: *c1cccc2c1[nH]c1ccccc12 ; *c1ccc2c(c1)[nH]c1ccccc12 ; *c1ccc2[nH]c3ccccc3c2c1 ; *c1cccc2[nH]c3ccccc3c12 ; *n1c2ccccc2c2ccccc21]

In this specification, the (9-phenyl)carbazolyl group is specifically any of the following groups, unless otherwise specified in this specification.

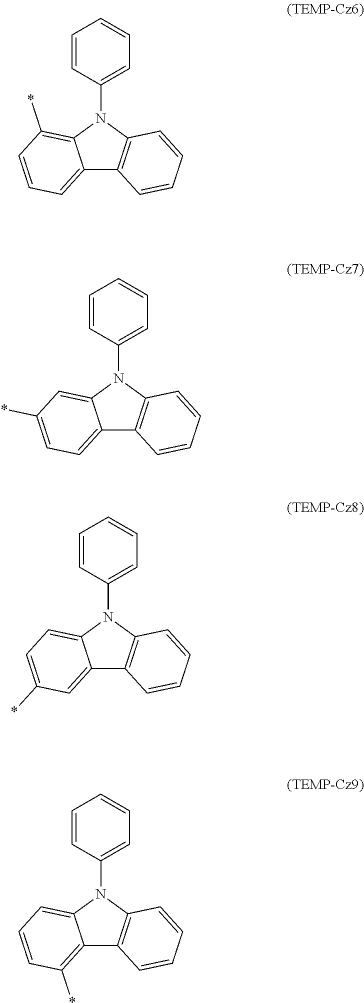 [SMILES: *c1cccc2c3ccccc3n(-c3ccccc3)c12 ; *c1ccc2c3ccccc3n(-c3ccccc3)c2c1 ; *c1ccc2c(c1)c1ccccc1n2-c1ccccc1 ; *c1cccc2c1c1ccccc1n2-c1ccccc1]

In the general formulas (TEMP-Cz1) to (TEMP-Cz9), * represents a bonding site.

In this specification, the dibenzofuranyl group and the dibenzothiophenyl group are specifically any of the following groups, unless otherwise specified in this specification.

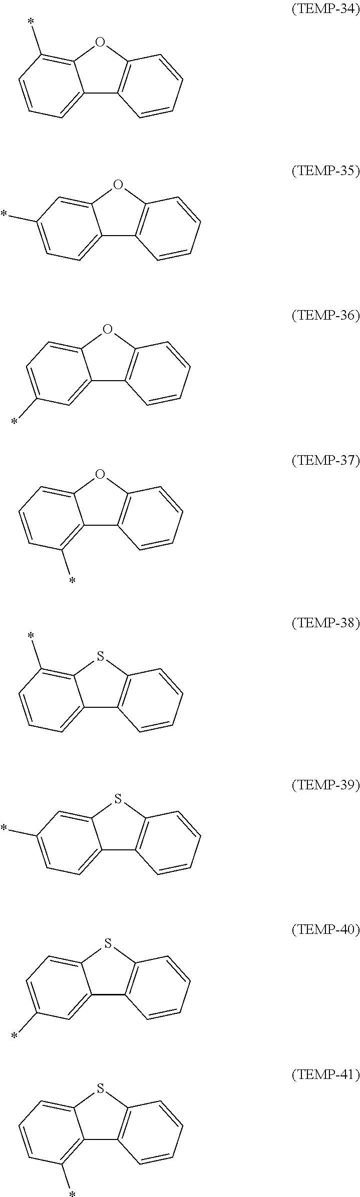

In the general formulas (TEMP-34) to (TEMP-41), * represents a bonding site.

The substituted or unsubstituted alkyl group described in this specification is preferably a methyl group, an ethyl group, a propyl group, an isopropyl group, a n-butyl group, an isobutyl group, a t-butyl group, or the like, unless otherwise specified in this specification.

### · “Substituted or Unsubstituted Arylene Group”

The “substituted or unsubstituted arylene group” described in this specification is a divalent group derived by removing one hydrogen atom on the aryl ring of the “substituted or unsubstituted aryl group”, unless otherwise specified. Specific examples of the “substituted or unsubstituted arylene group” (specific example group G12) include a divalent group derived by removing one hydrogen atom on the aryl ring of the “substituted or unsubstituted aryl group” described in the specific example group G1, and the like

### · “Substituted or Unsubstituted Divalent Heterocyclic Group”

The “substituted or unsubstituted divalent heterocyclic group” described in this specification is a divalent group derived by removing one hydrogen atom on the heterocycle of the “substituted or unsubstituted heterocyclic group”, unless otherwise specified. Specific examples of the “substituted or unsubstituted divalent heterocyclic group” (specific example group G13) include a divalent group derived by removing one hydrogen atom on the heterocycle of the “substituted or unsubstituted heterocyclic group” described in the specific example group G2, and the like.

### · “Substituted or Unsubstituted Alkylene Group”

The “substituted or unsubstituted alkylene group” described in this specification is a divalent group derived by removing one hydrogen atom on the alkyl chain of the “substituted or unsubstituted alkyl group”, unless otherwise specified. Specific examples of the “substituted or unsubstituted alkylene group” (specific example group G14) include a divalent group derived by removing one hydrogen atom on the alkyl chain of the “substituted or unsubstituted alkyl group” described in the specific example group G3, and the like.

The substituted or unsubstituted arylene group described in this specification is preferably any group of the following general formulas (TEMP-42) to (TEMP-68), unless otherwise specified in this specification.

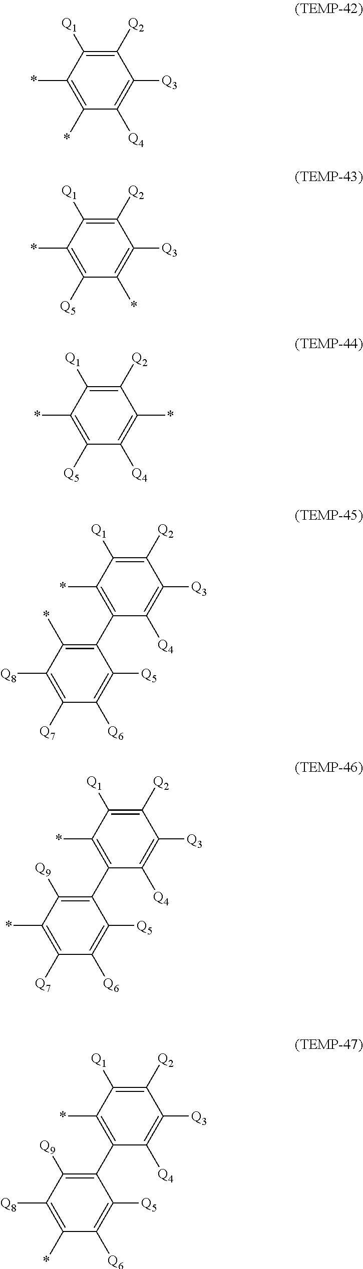

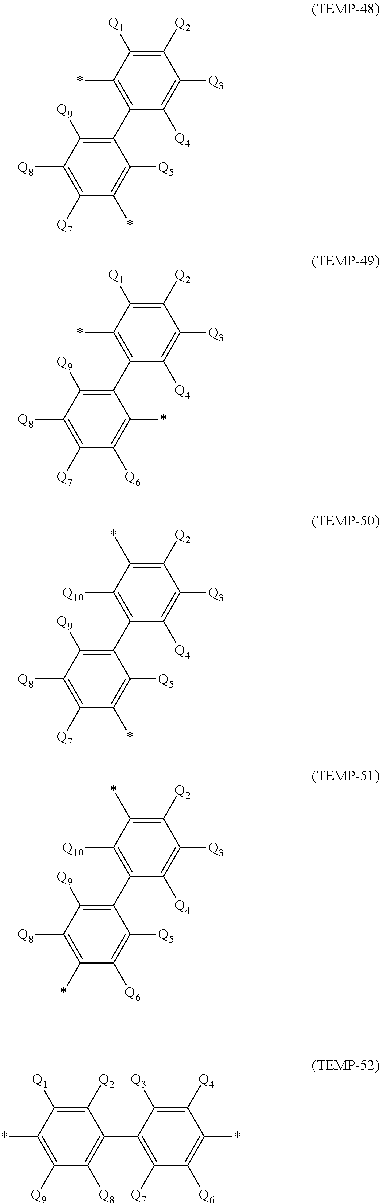

In the general formulas (TEMP-42) to (TEMP-52), Q₁ to Q₁₀ are independently a hydrogen atom or a substituent.

In the general formulas (TEMP-42) to (TEMP-52), * represents a bonding site.

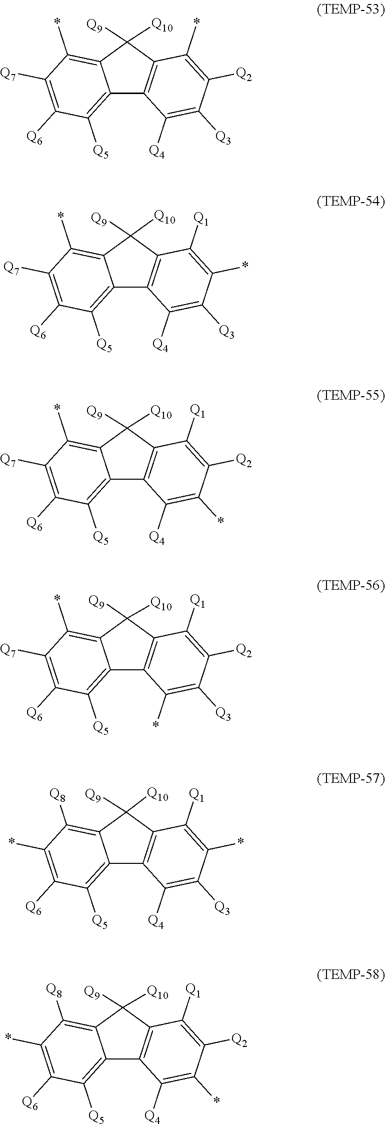

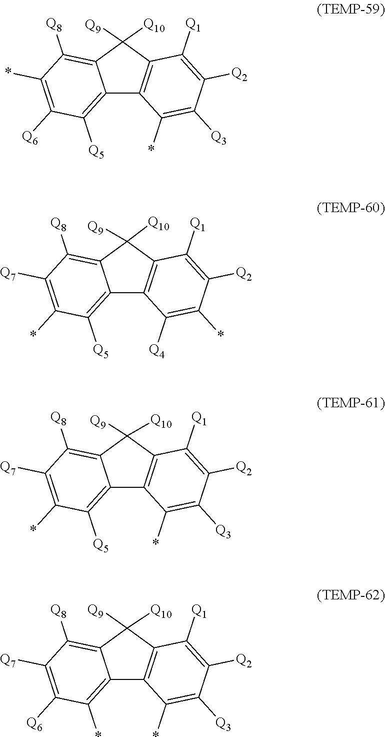

In the general formulas (TEMP-53) to (TEMP-62), Q₁ to Q₁₀ are independently a hydrogen atom or a substituent.

Q₉ and Q₁₀ may be bonded with each other via a single bond to form a ring.

In the general formulas (TEMP-53) to (TEMP-62), * represents a bonding site.

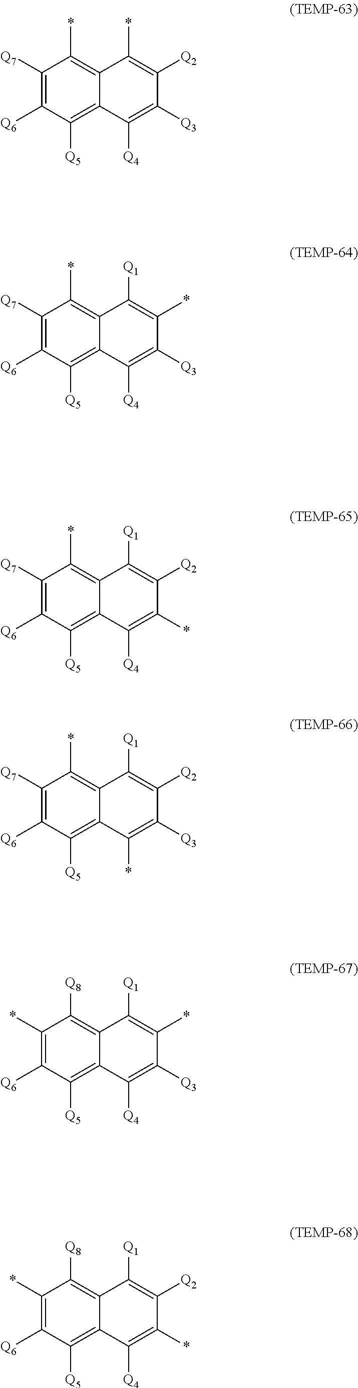

In the general formulas (TEMP-63) to (TEMP-68), Q₁ to Q₈ are independently a hydrogen atom or a substituent.

In the general formulas (TEMP-63) to (TEMP-68), * represents a bonding site.

The substituted or unsubstituted divalent heterocyclic group described in this specification is preferably any group of the following general formulas (TEMP-69) to (TEMP-102), unless otherwise specified in this specification.

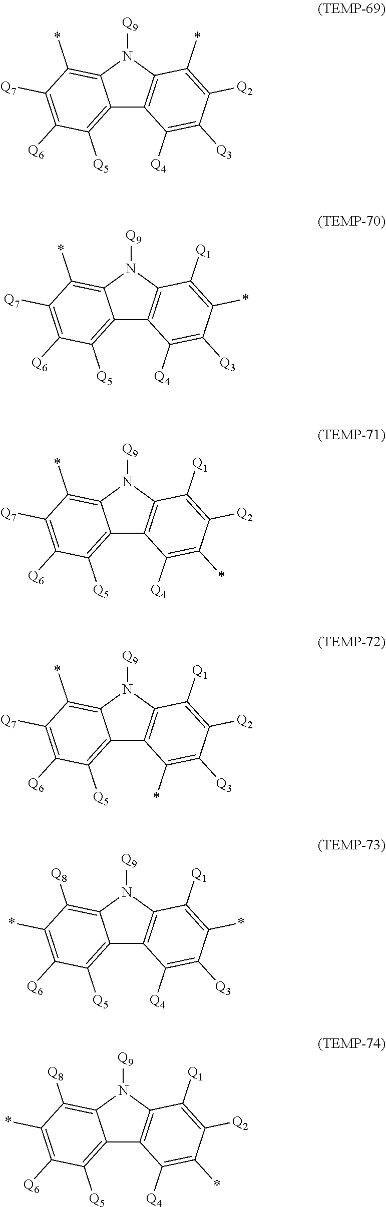

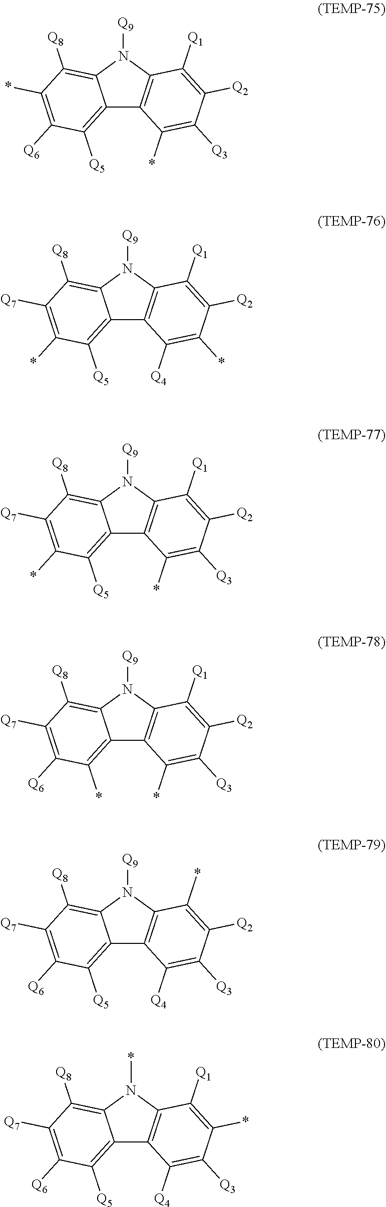

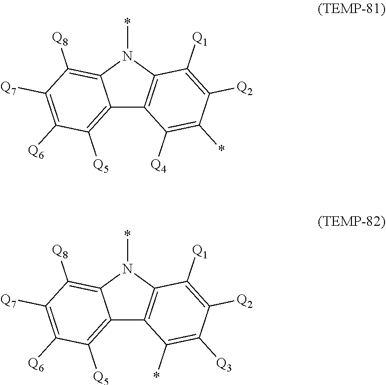

In the general formulas (TEMP-69) to (TEMP-82), Q₁ to Q₈ are independently a hydrogen atom or a substituent.

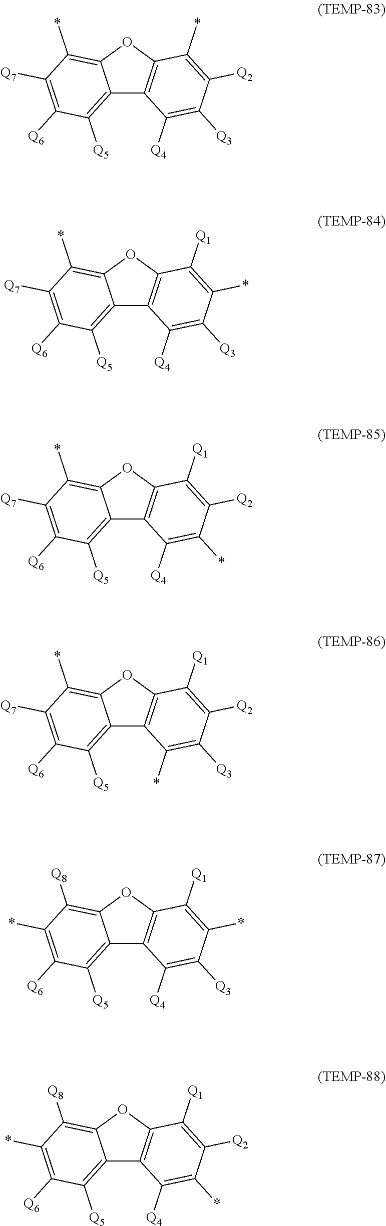

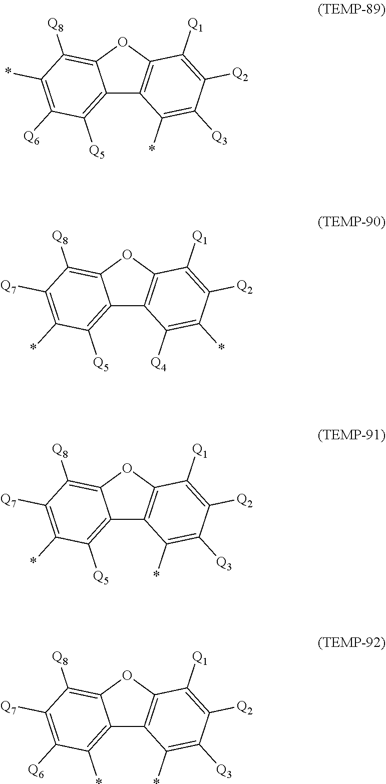

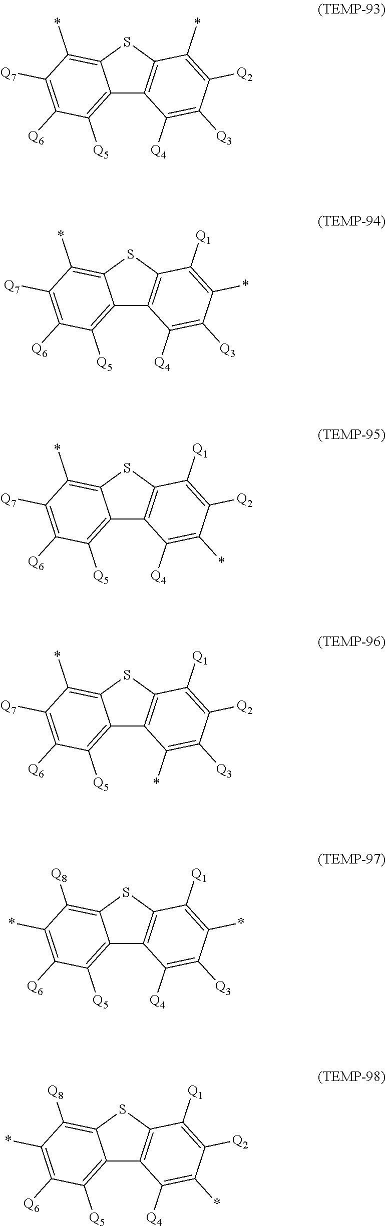

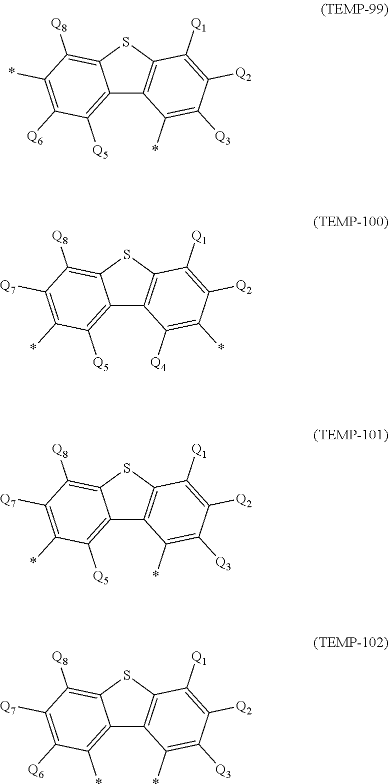

In the general formulas (TEMP-83) to (TEMP-102), Q₁ to Q₈ are independently a hydrogen atom or a substituent.

The above is the explanation of the “Substituent described in this specification.”

·“the Case where Bonded with Each Other to Form a Ring”

In this specification, the case where “one or more sets of adjacent two or more form a substituted or unsubstituted monocycle by bonding with each other, form a substituted or unsubstituted fused ring by bonding with each other, or do not bond with each other” means the case where “one or more sets of adjacent two or more form a substituted or unsubstituted monocycle by bonding with each other”; the case where “one or more sets of adjacent two or more form a substituted or unsubstituted fused ring by bonding with each other”; and the case where “one or more sets of adjacent two or more do not bond with each other.”

The case where “one or more sets of adjacent two or more form a substituted or unsubstituted monocycle by bonding with each other” and the case where “one or more sets of adjacent two or more form a substituted or unsubstituted fused ring by bonding with each other” in this specification (these cases may be collectively referred to as “the case where forming a ring by bonding with each other”) will be described below. The case of an anthracene compound represented by the following general formula (TEMP-103) in which the mother skeleton is an anthracene ring will be described as an example.

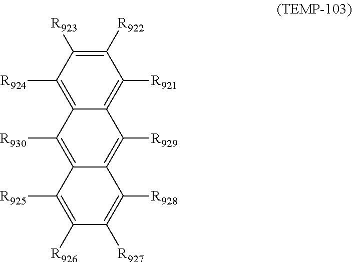

For example, in the case where “one or more sets of adjacent two or more among R₉₂₁ to R₉₃₀ form a ring by bonding with each other”, the one set of adjacent two includes a pair of R₉₂₁ and R₉₂₂, a pair of R₉₂₂ and R₉₂₃, a pair of R₉₂₃ and R₉₂₄, a pair of R₉₂₄ and R₉₃₀, a pair of R₉₃₀ and R₉₂₅, a pair of R₉₂₅ and R₉₂₆, a pair of R₉₂₆ and R₉₂₇, a pair of R₉₂₇ and R₉₂₈, a pair of R₉₂₈ and R₉₂₉, and a pair of R₉₂₉ and R₉₂₁.

The “one or more sets” means that two or more sets of the adjacent two or more sets may form a ring at the same time. For example, R₉₂₁ and R₉₂₂ form a ring Q_{A} by bonding with each other, and at the same, time R₉₂₅ and R₉₂₆ form a ring Q_{B} by bonding with each other, the anthracene compound represented by the general formula (TEMP-103) is represented by the following general formula (TEMP-104).

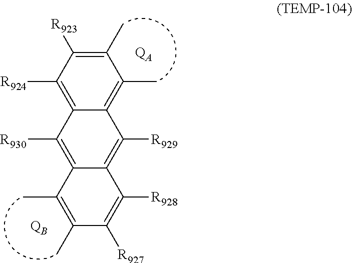

The case where the “set of adjacent two or more” form a ring includes not only the case where the set (pair) of adjacent “two” is bonded with as in the above-mentioned examples, but also the case where the set of adjacent “three or more” are bonded with each other. For example, it means the case where R₉₂₁ and R₉₂₂ form a ring Q_{A} by bonding with each other, and R₉₂₂ and R₉₂₃ form a ring Q_{C} by bonding with each other, and adjacent three (R₉₂₁, R₉₂₂ and R₉₂₃) form rings by bonding with each other and together fused to the anthracene mother skeleton. In this case, the anthracene compound represented by the general formula (TEMP-103) is represented by the following general formula (TEMP-105). In the following general formula (TEMP-105), the ring Q_{A} and the ring Q_{C} share R₉₂₂.

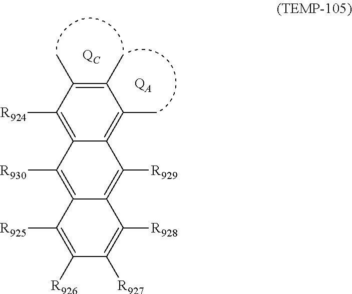

The “monocycle” or “fused ring” formed may be a saturated ring or an unsaturated ring, as a structure of the formed ring alone. Even when the “one pair of adjacent two” forms a “monocycle” or a “fused ring”, the “monocycle” or the “fused ring” may form a saturated ring or an unsaturated ring. For example, the ring Q_{A} and the ring Q_{B} formed in the general formula (TEMP-104) are independently a “monocycle” or a “fused ring.” The ring Q_{A} and the ring Q_{C} formed in the general formula (TEMP-105) are “fused ring.” The ring Q_{A} and ring Q_{C} of the general formula (TEMP-105) are fused ring by fusing the ring Q_{A} and the ring Q_{C} together. When the ring Q_{A} of the general formula (TEMP-104) is a benzene ring, the ring Q_{A} is a monocycle. When the ring Q_{A} of the general formula (TEMP-104) is a naphthalene ring, the ring Q_{A} is a fused ring.

The “unsaturated ring” includes, in addition to an aromatic hydrocarbon ring and an aromatic heterocycle, an aliphatic hydrocarbon ring with an unsaturated bond, i.e., double and/or triple bonds in the ring structure (e.g., cyclohexene, cyclohexadiene, etc.), and a non-aromatic heterocycle with an unsaturated bond (e.g., dihydropyran, imidazoline, pyrazoline, quinolizine, indoline, isoindoline, etc.). The “saturated ring” includes an aliphatic hydrocarbon ring without an unsaturated bond and a non-aromatic heterocycle without ab unsaturated bond.

Specific examples of the aromatic hydrocarbon ring include a structure in which the group listed as a specific example in the specific example group G1 is terminated by a hydrogen atom.

Specific examples of the aromatic heterocycle include a structure in which the aromatic heterocyclic group listed as a specific example in the example group G2 is terminated by a hydrogen atom.

Specific examples of the aliphatic hydrocarbon ring include a structure in which the group listed as a specific example in the specific example group G6 is terminated by a hydrogen atom.

The term “to form a ring” means forming a ring only with plural atoms of the mother skeleton, or with plural atoms of the mother skeleton and one or more arbitrary atoms in addition. For example, the ring Q_{A} Shown in the general formula (TEMP-104), which is formed by bonding R₉₂₁ and R₉₂₂ with each other, is a ring formed from the carbon atom of the anthracene skeleton with which R₉₂₁ is bonded, the carbon atom of the anthracene skeleton with which R₉₂₂ is bonded, and one or more arbitrary atoms. For example, in the case where the ring Q_{A} is formed with R₉₂₁ and R₉₂₂, when a monocyclic unsaturated ring is formed with the carbon atom of the anthracene skeleton with which R₉₂₁ is bonded, the carbon atom of the anthracene skeleton with which R₉₂₂ is bonded, and four carbon atoms, the ring formed with R₉₂₁ and R₉₂₂ is a benzene ring.

Here, the “arbitrary atom” is preferably at least one atom selected from the group consisting of a carbon atom, a nitrogen atom, an oxygen atom, and a sulfur atom, unless otherwise specified in this specification. In the arbitrary atom (for example, a carbon atom or a nitrogen atom), a bond which does not form a ring may be terminated with a hydrogen atom or the like, or may be substituted with “arbitrary substituent” described below. When an arbitrary atom other than a carbon atom is contained, the ring formed is a heterocycle.

The number of “one or more arbitrary atom(s)” constituting a monocycle or a fused ring is preferably 2 or more and 15 or less, more preferably 3 or more and 12 or less, and still more preferably 3 or more and 5 or less, unless otherwise specified in this specification.

The “monocycle” is preferable among the “monocycle” and the “fused ring”, unless otherwise specified in this specification.

The “unsaturated ring” is preferable among the “saturated ring” and the “unsaturated ring”, unless otherwise specified in this specification.

Unless otherwise specified in this specification, the “monocycle” is preferably a benzene ring.

Unless otherwise specified in this specification, the “unsaturated ring” is preferably a benzene ring.

Unless otherwise specified in this specification, when “one or more sets of adjacent two or more” are “bonded with each other to form a substituted or unsubstituted monocycle” or “bonded with each other to form a substituted or unsubstituted fused ring”, this specification, one or more sets of adjacent two or more are preferably bonded with each other to form a substituted or unsubstituted “unsaturated ring” from plural atoms of the mother skeleton and one or more and 15 or less atoms which is at least one kind selected from a carbon atom, a nitrogen atom, an oxygen atom, and a sulfur atom.

The substituent in the case where the above-mentioned “monocycle” or “fused ring” has a substituent is, for example, an “arbitrary substituent” described below. Specific examples of the substituent which the above-mentioned “monocycle” or “fused ring” has include the substituent described above in the “Substituent described in this specification” section.

The substituent in the case where the above-mentioned “saturated ring” or “unsaturated ring” has a substituent is, for example, an “arbitrary substituent” described below. Specific examples of the substituent which the above-mentioned “monocycle” or “fused ring” has include the substituent described above in the “Substituent described in this specification” section.

The foregoing describes the case where “one or more sets of adjacent two or more form a substituted or unsubstituted monocycle by bonding with each other” and the case where “one or more sets of adjacent two or more form a substituted or unsubstituted fused ring by bonding with each other” (the case where “forming a ring by bonding with each other”).

### ·Substituent in the Case of “Substituted or Unsubstituted”

In one embodiment in this specification, the substituent (in this specification, sometimes referred to as an “arbitrary substituent”) in the case of “substituted or unsubstituted” is, for example, a group selected from the group consisting of:
an unsubstituted alkyl group including 1 to 50 carbon atoms,
an unsubstituted alkenyl group including 2 to 50 carbon atoms,
an unsubstituted alkynyl group including 2 to 50 carbon atoms,
an unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
an unsubstituted aryl group including 6 to 50 ring carbon atoms, and
an unsubstituted heterocyclic group including 5 to 50 ring atoms,
wherein, R₉₀₁ to R₉₀₇ are independently
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted heterocyclic group including 5 to 50 ring atoms.

When two or more R₉₀₁'s are present, the two or more R₉₀₁'s may be the same or different.

When two or more R₉₀₂'s are present, the two or more R₉₂'s may be the same or different.

When two or more R₉₀₃'s are present, the two or more R₉₀₃'s may be the same or different.

When two or more R₉₀₄'s are present, the two or more R₉₀₄'s may be the same or different.

When two or more R₉₀₅'s are present, the two or more R₉₀₅'s may be the same or different.

When two or more R₉₀₆'s are present, the two or more R₉₀₆'s may be the same or different.

When two or more R₉₀₇'s are present, the two or more R₉₀₇'s may be the same or different.

In one embodiment, the substituent in the case of “substituted or unsubstituted” is a group selected from the group consisting of:
an alkyl group including 1 to 50 carbon atoms,
an aryl group including 6 to 50 ring carbon atoms, and
a heterocyclic group including 5 to 50 ring atoms.

In one embodiment, the substituent in the case of “substituted or unsubstituted” is a group selected from the group consisting of:
an alkyl group including 1 to 18 carbon atoms,
an aryl group including 6 to 18 ring carbon atoms, and
a heterocyclic group including 5 to 18 ring atoms.

Specific examples of each of the arbitrary substituents include specific examples of substituent described in the section “Substituent described in this specification” above.

Unless otherwise specified in this specification, adjacent arbitrary substituents may form a “saturated ring” or an “unsaturated ring”, preferably form a substituted or unsubstituted saturated 5-membered ring, a substituted or unsubstituted saturated 6-membered ring, a substituted or unsubstituted unsaturated 5-membered ring, or a substituted or unsubstituted unsaturated 6-membered ring, more preferably form a benzene ring.

Unless otherwise specified in this specification, the arbitrary substituent may further have a substituent. The substituent which the arbitrary substituent further has is the same as that of the above-mentioned arbitrary substituent.

In this specification, the numerical range represented by “AA to BB” means the range including the numerical value AA described on the front side of “AA to BB” as the lower limit and the numerical value BB described on the rear side of “AA to BB” as the upper limit.

Although only some exemplary embodiments and/or examples of this invention have been described in detail above, those skilled in the art will readily appreciate that many modifications are possible in the exemplary embodiments and/or examples without materially departing from the novel teachings and advantages of this invention. Accordingly, all such modifications are intended to be included within the scope of this invention.

The documents described in the specification and the specification of Japanese application(s) on the basis of which the present application claims Paris convention priority are incorporated herein by reference in its entirety.

1. A mixed powder comprising a first compound and a second compound,
wherein the first compound has the fused ring structure having the most rings and having three or more rings, in fused ring structures included therein, and
the second compound has the fused ring structure having the most rings and having 5 or more rings, in fused ring structures included therein, and the fused ring structure having the most rings thereof has more rings than that of the first compound.

2. The mixed powder according to claim 1, wherein the first compound is a compound which has the fused ring structure having the most rings and having three or four rings, in fused ring structures included therein.

3. The mixed powder according to claim 1, wherein the first compound is one or more compounds selected from the group consisting of compounds represented by the following formulas (2) and (H11) to (H16): 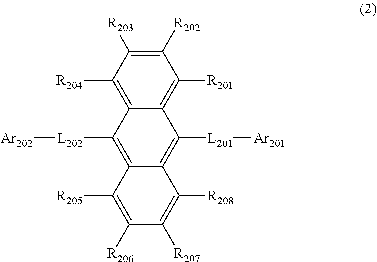
wherein in the formula (2),
R₂₀₁ to R₂₀₈ are independently
a hydrogen atom,
a substituted or unsubstituted alkyl group having 1 to 50 carbon atoms,
a substituted or unsubstituted haloalkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —Si(R₉₀₁)(R₉₀₂)(R₉₀₃),
a group represented by —O—(R₉₀₄),
a group represented by —S—(R₉₀₅),
a group represented by —N(R₉₀₆)(R₉₀₇),
a substituted or unsubstituted aralkyl group having 7 to 50 carbon atoms,
a group represented by —C(═O)R₈₀₁,
a group represented by —COOR₈₀₂,
a halogen atom,
a cyano group,
a nitro group,
a substituted or unsubstituted aryl group having 6 to 50 ring carbon atoms, or
a substituted or unsubstituted heterocyclic group having 5 to 50 ring atoms;
L₂₀₁ and L₂₀₂ are independently
a single bond,
a substituted or unsubstituted arylene group having 6 to 50 ring carbon atoms, or
a substituted or unsubstituted divalent heterocyclic group having 5 to 50 ring atoms;
Ar₂₀₁ and Ar₂₀₂ are independently
a substituted or unsubstituted aryl group having 6 to 50 ring carbon atoms, or
a substituted or unsubstituted heterocyclic group having 5 to 50 ring atoms;
R₉₀₁ to R₉₀₇, R₈₀₁ and R₈₀₂ are independently
a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted heterocyclic group having 5 to 50 ring atoms;
when a plurality of R₉₀₁'s is present, the plurality of R₉₀₁'s is the same as or different from each other;
when a plurality of R₉₀₂'s is present, the plurality of R₉₀₂'s is the same as or different from each other;
when a plurality of R₉₀₃'s is present, the plurality of R₉₀₃'s is the same as or different from each other;
when a plurality of R₉₀₄'s is present, the plurality of R₉₀₄'s is the same as or different from each other;
when a plurality of R₉₀₅'s is present, the plurality of R₉₀₅'s is the same as or different from each other;
when a plurality of R₉₀₆'s is present, the plurality of R₉₀₆'s is the same as or different from each other;
when a plurality of R₉₀₇'s is present, the plurality of R₉₀₇'s is the same as or different from each other;
when a plurality of R₈₀₁'s is present, the plurality of R₈₀₁'s is the same as or different from each other; and
when a plurality of R₈₀₂'s is present, the plurality of R₈₀₂'s is the same as or different from each other: 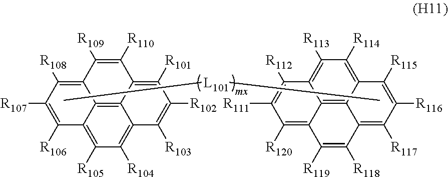
wherein in the formula (H11),
R₁₀₁ to R₁₁₀, and R₁₁₁ to R₁₂₀ are independently
a hydrogen atom,
a substituted or unsubstituted alkyl group having 1 to 50 carbon atoms,
a substituted or unsubstituted haloalkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —Si(R₉₀₁)(R₉₀₂)(R₉₀₃),
a group represented by —O—(R₉₀₄),
a group represented by —S—(R₉₀₅),
a substituted or unsubstituted aralkyl group having 7 to 50 carbon atoms,
a group represented by —C(═O)R₈₀₁,
a group represented by —COOR₈₀₂,
a halogen atom,
a cyano group,
a nitro group,
a substituted or unsubstituted aryl group having 6 to 50 ring carbon atoms, or
a substituted or unsubstituted heterocyclic group having 5 to 50 ring atoms;
provided that one of R₁₀₁ to R₁₁₀ represents a bonding position with L₁₀₁, and one of R₁₁₁ to R₁₂₀ represents a bonding position with L₁₀₁;
L₁₀₁ is
a single bond,
a substituted or unsubstituted arylene group having 6 to 24 ring carbon atoms, or
a substituted or unsubstituted divalent heterocyclic group having 5 to 24 ring atoms;
mx is 0, 1, 2, 3, 4 or 5;
when two or more Lion's are present, the two or more Lion's are the same as or different from each other;
R₉₀₁ to R₉₀₅, R₈₀₁ and R₈₀₂ are independently the same as defined for R₉₀₁ to R₉₀₅, R₈₀₁ and R₈₀₂ in the formula (2): 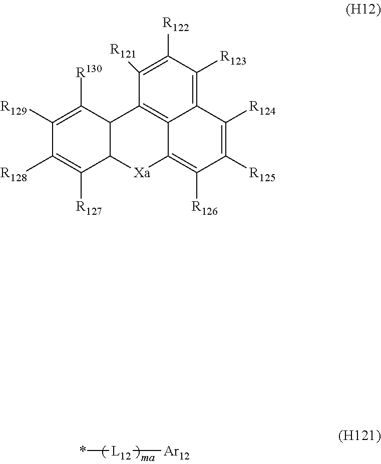
wherein in the formula (H12),
Xa is an oxygen atom, a sulfur atom, C(R₁₂₀₁)(R₁₂₀₂), or Si(R₁₂₀₃)(R₁₂₀₄);
R₁₂₀₁ to R₁₂₀₄ are independently
a hydrogen atom,
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —Si(R₉₀₁)(R₉₀₂)(R₉₀₃),
a group represented by —O—(R₉₀₄),
a group represented by —S—(R₉₀₅),
a group represented by —N(R₉₀₆)(R₉₀₇),
a halogen atom,
a cyano group,
a nitro group,
a substituted or unsubstituted aryl group having 6 to 50 ring carbon atoms, or
a substituted or unsubstituted heterocyclic group having 5 to 50 ring atoms;
one or more sets of the adjacent two or more of R₁₂₁ to R₁₃₀
form a substituted or unsubstituted single ring by bonding with each other,
form a substituted or unsubstituted fused ring by bonding with each other, or
do not bond with each other;
R₁₂₁ to R₁₃₀ which do not form the substituted or unsubstituted single ring and which do not form the substituted or unsubstituted fused ring are independently
a hydrogen atom,
a substituted or unsubstituted alkyl group having 1 to 50 carbon atoms,
a substituted or unsubstituted haloalkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —Si(R₉₀₁)(R₉₀₂)(R₉₀₃),
a group represented by —O—(R₉₀₄),
a group represented by —S—(R₉₀₅),
a group represented by —N(R₉₀₆)(R₉₀₇),
a substituted or unsubstituted aralkyl group having 7 to 50 carbon atoms,
a group represented by —C(═O)R₈₀₁,
a group represented by —COOR₈₀₂,
a halogen atom,
a nitro group,
a substituted or unsubstituted aryl group having 6 to 50 ring carbon atoms,
a substituted or unsubstituted heterocyclic group having 5 to 50 ring atoms, or
a group represented by the formula (H121);
provided that at least one of R₁₂₁ to R₁₃₀ is the group represented by the formula (H121);
when a plurality of groups represented by the formula (H121) is present, the plurality of groups represented by the formula (H121) is the same as or different from each other;
L₁₂ is
a single bond,
a substituted or unsubstituted arylene group having 6 to 50 ring carbon atoms, or
a substituted or unsubstituted divalent heterocyclic group having 5 to 50 ring atoms;
ma is 0, 1, 2 or 3;
when two or more L₁₂'s are present, the two or more L₁₂'s are the same as or different from each other;
Ar₁₂ is a substituted or unsubstituted aryl group having 6 to 50 ring carbon atoms, or a substituted or unsubstituted heterocyclic group having 5 to 50 ring atoms;
when two or more Ar₁₂'s are present, the two or more Ar₁₂'s are the same as or different from each other;
* in the formula (H121) represents a bonding position;
R₉₀₁ to R₉₀₇, R₈₀₁ and R₈₀₂ are independently the same as defined for R₉₀₁ to R₉₀₇, R₈₀₁ and R₈₀₂ in the formula (2): 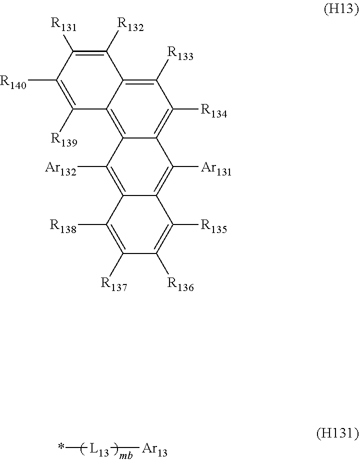
wherein in the formula (H13),
R₁₃₁ to R₁₄₀, Ar₁₃₁ and Ar₁₃₂ are independently
a hydrogen atom,
a substituted or unsubstituted alkyl group having 1 to 50 carbon atoms,
a substituted or unsubstituted haloalkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —Si(R₉₀₁)(R₉₀₂)(R₉₀₃),
a group represented by —O—(R₉₀₄),
a group represented by —S—(R₉₀₅),
a substituted or unsubstituted aralkyl group having 7 to 50 carbon atoms,
a group represented by —C(═O)R₈₀₁,
a group represented by —COOR₈₀₂,
a halogen atom,
a cyano group,
a nitro group,
a substituted or unsubstituted aryl group having 6 to 50 ring carbon atoms,
a substituted or unsubstituted heterocyclic group having 5 to 50 ring atoms, or
a group represented by the formula (H131);
provided that at least one of R₁₃₁ to R₁₄₀, Ar₁₃₁ and Ar₁₃₂ is the group represented by the formula (H131);
when a plurality of groups represented by the formula (H131) is present, the plurality of groups represented by the formula (H131) is the same as or different from each other;
L₁₃ is
a single bond,
a substituted or unsubstituted arylene group having 6 to 50 ring carbon atoms, or
a substituted or unsubstituted divalent heterocyclic group having 5 to 50 ring atoms;
Ar₁₃ is
a substituted or unsubstituted aryl group having 6 to 50 ring carbon atoms, or
a substituted or unsubstituted heterocyclic group having 5 to 50 ring atoms;
mb is 0, 1, 2, 3, 4 or 5;
when two or more L₁₃'s are present, the two or more L₁₃'s are the same as or different from each other;
when two or more Ar₁₃'s are present, the two or more Ar₁₃'s are the same as or different from each other;
* in the formula (H131) represents a bonding position with the benz[a]anthracene ring in the formula (H13);
R₉₀₁ to R₉₀₅, R₈₀₁ and R₈₀₂ are independently the same as defined for R₉₀₁ to R₉₀₅, R₈₀₁ and R₈₀₂ in the formula (2): 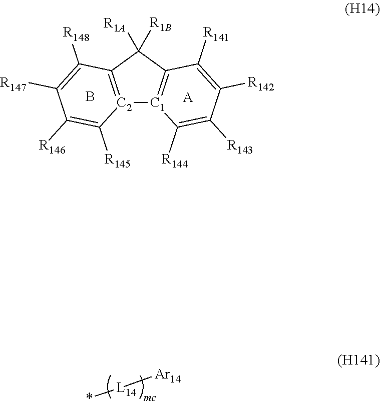
wherein in the formula (H14),
R_{1A} and R_{1B} are independently
a substituted or unsubstituted alkyl group having 1 to 15 carbon atoms,
a substituted or unsubstituted aryl group having 6 to 17 ring carbon atoms, or
a substituted or unsubstituted heterocyclic group having 5 to 17 ring atoms;
provided that at least one of R_{1A} and R_{1B} is a substituted or unsubstituted alkyl group having 1 to 15 carbon atoms;
any one set of the adjacent two or more of R₁₄₁ to R₁₄₄ and the adjacent two or more of R₁₄₅ to R₁₄₈
forms a substituted or unsubstituted single ring by bonding with each other, or
forms a substituted or unsubstituted fused ring by bonding with each other,
a group represented by the formula (H141)
is bonded with the carbon atom with which R₁₄₂ is bonded, or a carbon atom which is farthest from the carbon atom C₁ in the ring A being bonded with the carbon atom C₂ in the ring B via a single bond, of carbon atoms constituting the single ring on the ring A side and the fused ring on the ring A side, in the case where the substituted or unsubstituted single ring or the substituted or unsubstituted fused ring is formed on the ring A side, or
is bonded with the carbon atom with which R₁₄₂ is bonded in the case where the substituted or unsubstituted single ring or the substituted or unsubstituted fused ring is not formed on the ring A side, but is formed on the ring B side;
R₁₄₂ which are not the group represented by the formula (H141), and R₁₄₁, R₁₄₃, R₁₄₄ and R₁₄₅ to R₁₄₈ which do not form the substituted or unsubstituted single ring and which do not form the substituted or unsubstituted fused ring are independently
a hydrogen atom,
a substituted or unsubstituted alkyl group having 1 to 50 carbon atoms,
a substituted or unsubstituted haloalkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —Si(R₉₀₁)(R₉₀₂)(R₉₀₃),
a group represented by —O—(R₉₀₄),
a group represented by —S—(R₉₀₅),
a group represented by —N(R₉₀₆)(R₉₀₇),
a substituted or unsubstituted aralkyl group having 7 to 50 carbon atoms,
a group represented by —C(═O)R₈₀₁,
a group represented by —COOR₈₀₂,
a halogen atom,
a cyano group,
a nitro group,
a substituted or unsubstituted aryl group having 6 to 17 ring carbon atoms, or
a substituted or unsubstituted heterocyclic group having 5 to 17 ring atoms;
in the formula (H141),
Ar₁₄ is a substituted or unsubstituted aryl group in which four or more rings are fused, or a substituted or unsubstituted heterocyclic group in which four or more rings are fused;
L₁₄ is
a single bond,
a substituted or unsubstituted arylene group having 6 to 17 ring carbon atoms, or
a substituted or unsubstituted divalent heterocyclic group having 5 to 17 ring atoms;
mc is 0, 1 or 2;
* represents a bonding position with an atom constituting the ring of the formula (H14);
provided that the compound represented by the formula (H14) does not contain three or more groups of a substituted or unsubstituted aryl group in which four or more rings are fused, and a substituted or unsubstituted heterocyclic group in which four or more rings are fused in the molecule of the compound represented by the formula (H14);
R₉₀₁ to R₉₀₇, R₈₀₁ and R₈₀₂ are independently the same as defined for R₉₀₁ to R₉₀₇, R₈₀₁ and R₈₀₂ in the formula (2): 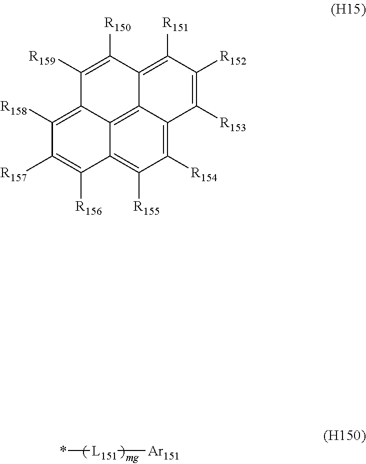
wherein in the formula (H15),
R₁₅₀ to R₁₅₉ are independently
a hydrogen atom,
a substituted or unsubstituted alkyl group having 1 to 50 carbon atoms,
a substituted or unsubstituted haloalkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —Si(R₉₀₁)(R₉₀₂)(R₉₀₃),
a group represented by —O—(R₉₀₄),
a group represented by —S—(R₉₀₅),
a substituted or unsubstituted aralkyl group having 7 to 50 carbon atoms,
a group represented by —C(═O)R₈₀₁,
a group represented by —COOR₈₀₂,
a halogen atom,
a cyano group,
a nitro group,
a substituted or unsubstituted aryl group having 6 to 50 ring carbon atoms,
a substituted or unsubstituted heterocyclic group having 5 to 50 ring atoms, or
a group represented by the formula (H150);
provided that at least one of R₁₅₀ to R₁₅₉ is the group represented by the formula (H150);
when a plurality of groups represented by the formula (H150) is present, the plurality of groups represented by the formula (H150) is the same as or different from each other;
L₁₅₁ is
a single bond,
a substituted or unsubstituted arylene group having 6 to 50 ring carbon atoms, or
a substituted or unsubstituted divalent heterocyclic group having 5 to 50 ring atoms;
Ar₁₅₁ is
a substituted or unsubstituted aryl group having 6 to 50 ring carbon atoms, or
a substituted or unsubstituted heterocyclic group having 5 to 50 ring atoms;
mg is 0, 1, 2, 3, 4 or 5;
when two or more L₁₅₁'s are present, the two or more L₁₅₁'s are the same as or different from each other;
when two or more Ar₁₅₁'s are present, the two or more Ar₁₅₁'s are the same as or different from each other;
* in the formula (H150) represents a bonding position with the pyrene ring in the formula (H15);
R₉₀₁ to R₉₀₅, R₈₀₁ and R₈₀₂ are independently the same as defined for R₉₀₁ to R₉₀₅, R₈₀₁ and R₈₀₂ in the formula (2): 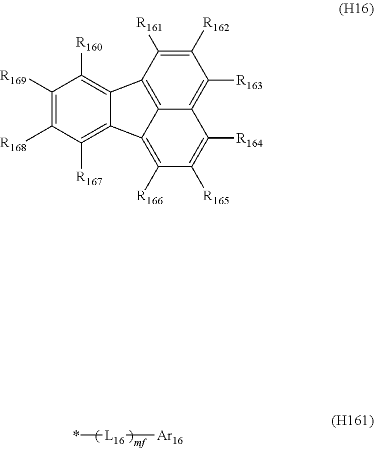
wherein in the formula (H16),
one or more sets of the adjacent two or more of R₁₆₀ to R₁₆₉
form a substituted or unsubstituted single ring by bonding with each other,
form a substituted or unsubstituted fused ring by bonding with each other, or
do not bond with each other;
R₁₆₀ to R₁₆₉ which do not form the substituted or unsubstituted single ring and which do not form the substituted or unsubstituted fused ring are independently
a hydrogen atom,
a substituted or unsubstituted alkyl group having 1 to 50 carbon atoms,
a substituted or unsubstituted haloalkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —Si(R₉₀₁)(R₉₀₂)(R₉₀₃),
a group represented by —O—(R₉₀₄),
a group represented by —S—(R₉₀₅),
a substituted or unsubstituted aralkyl group having 7 to 50 carbon atoms,
a group represented by —C(═O)R₈₀₁,
a group represented by —COOR₈₀₂,
a halogen atom,
a cyano group,
a nitro group,
a substituted or unsubstituted aryl group having 6 to 50 ring carbon atoms,
a substituted or unsubstituted heterocyclic group having 5 to 50 ring atoms, or
a group represented by the formula (H161);
provided that at least one of the substituent in the case where the substituted or unsubstituted single ring has the substituent, the substituent in the case where the substituted or unsubstituted fused ring has the substituent, and R₁₆₀ to R₁₆₉ is the group represented by the formula (H161);
when a plurality of groups represented by the formula (H161) is present, the plurality of groups represented by the formula (H161) is the same as or different from each other;
L₁₆ is
a single bond,
a substituted or unsubstituted arylene group having 6 to 50 ring carbon atoms, or
a substituted or unsubstituted divalent heterocyclic group having 5 to 50 ring atoms;
Ar₁₆ is
a substituted or unsubstituted aryl group having 6 to 50 ring carbon atoms, or
a substituted or unsubstituted heterocyclic group having 5 to 50 ring atoms;
mf is 0, 1, 2, 3, 4 or 5;
when two or more L₁₆'s are present, the two or more L₁₆'s are the same as or different from each other;
when two or more Ar₁₆'s are present, the two or more Ar₁₆'s are the same as or different from each other;
* in the formula (H161) represents a bonding position with the ring represented by the formula (H16); and
R₉₀₁ to R₉₀₅, R₈₀₁ and R₈₀₂ are independently the same as defined for R₉₀₁ to R₉₀₅, R₈₀₁ and R₈₀₂ in the formula (2).

4. The mixed powder according to claim 1, wherein the second compound is a compound which has the fused ring structure having the most rings and having 5 to 10 rings, in fused ring structures included therein.

5. The mixed powder according to claim 1, wherein the second compound is one or more compounds selected from the group consisting of compounds represented by the following formulas (5) and (6): 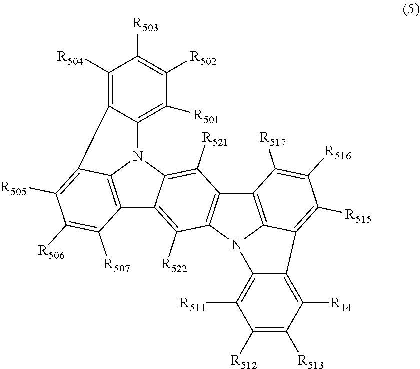
wherein in the formula (5),
one or more sets of the adjacent two or more of R₅₀₁ to R₅₀₇ and R₅₁₁ to R₅₁₇
form a substituted or unsubstituted single ring by bonding with each other,
form a substituted or unsubstituted fused ring by bonding with each other, or
do not bond with each other;
R₅₀₁ to R₅₀₇ and R₅₁₁ to R₅₁₇ which do not form the single ring and which do not form the fused ring are independently
a hydrogen atom,
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —Si(R₉₀₁)(R₉₀₂)(R₉₀₃),
a group represented by —O—(R₉₀₄),
a group represented by —S—(R₉₀₅),
a group represented by —N(R₉₀₆)(R₉₀₇),
a halogen atom,
a cyano group,
a nitro group,
a substituted or unsubstituted aryl group having 6 to 50 ring carbon atoms, or
a substituted or unsubstituted heterocyclic group having 5 to 50 ring atoms;
R₅₂₁ and R$22 are independently
a hydrogen atom,
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —Si(R₉₀₁)(R₉₀₂)(R₉₀₃),
a group represented by —O—(R₉₀₄),
a group represented by —S—(R₉₀₅),
a group represented by —N(R₉₀₆)(R₉₀₇),
a halogen atom,
a cyano group,
a nitro group,
a substituted or unsubstituted aryl group having 6 to 50 ring carbon atoms, or
a substituted or unsubstituted heterocyclic group having 5 to 50 ring atoms;
R₉₀₁ to R₉₀₇ are independently the same as defined for R₉₀₁ to R₉₀₇ in the formula (2): 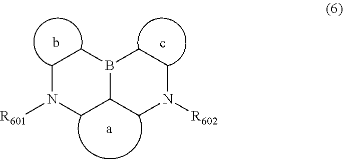
wherein in the formula (6),
a ring a, a ring b and a ring c are independently
a substituted or unsubstituted aromatic hydrocarbon ring having 6 to 50 ring carbon atoms, or
a substituted or unsubstituted heterocycle having 5 to 50 ring atoms;
R₆₀₁ and R₆₀₂ independently form a substituted or unsubstituted heterocycle by bonding with the ring a, the ring b or the ring c, or do not form the substituted or unsubstituted heterocycle; and
R₆₀₁ and R₆₀₂ which do not form the substituted or unsubstituted heterocycle are independently
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted heterocyclic group having 5 to 50 ring atoms.

6. The mixed powder according to claim 1, wherein the second compound is a compound in which the orientation order parameter S′ obtained by the following formula is 0.8 or more in a thin film formed using a vacuum deposition process:
$${S}^{′} = 1 - < cos 2 θ > = 2 ko / ( ke + 2 k o ) = 2 / 3 ( 1 - S )$$
$$S = ( 1 / 2 ) < 3 cos 2 θ - 1 > = ( k e - ko ) / ( ke + 2 k o )$$
wherein θ is the angle between the molecular axis in the thin film and the normal direction to a substrate; ko is the extinction coefficient in the direction parallel to the substrate obtained by multi-incident angle spectroscopic ellipsometry measurement of the thin film; and ke is the extinction coefficient in the vertical direction thereto obtained by multi-incident angle spectroscopic ellipsometry measurement of the thin film.

7. The mixed powder according to claim 1, wherein when MOL1 [mol %] is used as the molar concentration of the first compound in the mixed powder and MOL2 [mol %] is used as the molar concentration of the second compound in the mixed powder, MOL1 [mol %] and MOL2 [mol %] satisfy the following formula (1):
$$0 < MOL 2 / ( MOL 1 + M O L 2 ) ≦ 0.2 . \qquad ( 1 )$$

8. The mixed powder according to claim 1, wherein the amount of the second compound is 10% by mass or less based on the total amount of the first compound and the second compound.

9. The mixed powder according to claim 1, wherein the amount of the second compound is 5% by mass or less based on the total amount of the first compound and the second compound.

10. The mixed powder according to claim 7, wherein when MOL1 [mol %] is used as the molar concentration of the first compound in the mixed powder and MOL2 [mol %] is used as the molar concentration of the second compound in the mixed powder,
MOL1 [mol %] and MOL2 [mol %] satisfy the following formula (1), and
when M1 [kg/mol] is used as the molecular weight of the first compound,
when P1 [Pa] is used as the vapor pressure obtained by ThermoGravimetry-Differential Thermal Analysis of the first compound at arbitrary temperature,
when M2 [kg/mol] is used as the molecular weight of the second compound, and
when P2 [Pa] is used as the vapor pressure obtained by ThermoGravimetry-Differential Thermal Analysis of the second compound at arbitrary temperature,
the temperature T1 [° C.] of the first compound in the case of P1/M1^{1/2}=0.04×{MOL1/(MOL1+MOL2)} and
the temperature T2 [° C.] of the second compound in the case of P2/M2^{1/2}=0.04×{MOL2/(MOL1+MOL2)} satisfy the following formula (2):
$$0 < MOL 2 / ( MOL 1 + M O L 2 ) ≦ 0.2 \qquad ( 1 )$$
$$- 2 {0}^{°} C . ≦ T 1 - T 2 ≦ 4 {0}^{°} C . \qquad ( 2 )$$

11. The mixed powder according to claim 7, wherein 5% weight loss temperature T_{d5}1 obtained by ThermoGravimetry-Differential Thermal Analysis of the first compound is lower than 5% weight loss temperature T_{d5}2 obtained by ThermoGravimetry-Differential Thermal Analysis of the second compound.

12. The mixed powder according to claim 1, which is for an organic electroluminescence device.

13. The mixed powder according to claim 1, wherein 99.9 mol % or more thereof is the first compound and the second compound.

14. The mixed powder according to claim 1, which substantially consists of the first compound and the second compound.

15. The mixed powder according to claim 1, which consists of the first compound and the second compound.

16. The mixed powder according to claim 1, which is solid-like.

17. The mixed powder according to claim 1, which is pellet-like.

18. A mixture obtained by heating and melting the mixed powder according to claim 1, and by solidifying it.

19. A method for fabricating an organic electroluminescence device, wherein the organic electroluminescence device comprises a cathode, an anode, and one or two or more organic layers arranged between the cathode and the anode, and comprising an emitting layer, and
the method comprises: depositing a mixed powder by heating and vaporizing the mixed powder which comprises a first compound and a second compound, which is solid at ordinary temperatures and pressures, and in which the first compound has the fused ring structure having the most rings and having three or more rings, in fused ring structures included therein, and the second compound has the fused ring structure having the most rings and having 5 or more rings, in fused ring structures included therein, and the fused ring structure having the most rings thereof has more rings than that of the first compound in a deposition source to form at least one layer of the one or two or more organic layers.
